(19) United States
(12) Patent Application Publication
(10) Pub. No.: US 2026/0258423 A1
(43) Pub. Date: Sep. 3, 2026

(54) GAPMER ANTISENSE OLIGONUCLEOTIDES WITH MODIFIED BACKBONE CHEMISTRIES

(71) Applicant: QURALIS CORPORATION, Cambridge, MA (US)

(72) Inventors: SANDRA HINCKLEY, Cambridge, MA (US); DUNCAN BROWN, Berkeley, CA (US); DANIEL ELBAUM, Cambridge, MA (US)

(21) Appl. No.: 18/715,682

(22) PCT Filed: Dec. 2, 2022
(86) PCT No.: PCT/US2022/051663

§ 371 Date: Jun. 3, 2024

Related U.S. Application Data
(60) Provisional application No. 63/285,665, filed on Dec. 3, 2021.
(60) Provisional application No. 63/285,692, filed on Dec. 3, 2021.
(60) Provisional application No. 63/285,696, filed on Dec. 3, 2021.
(60) Provisional application No. 63/285,705, filed on Dec. 3, 2021.
(60) Provisional application No. 63/285,888, filed on Dec. 3, 2021.

(51) Int. Cl. C12N 15/113; A61K 31/7115; A61K 31/712; A61K 31/7125; A61K 45/06

(52) U.S. Cl. CPC C12N 15/1137; A61K 31/7115; A61K 31/712; A61K 31/7125; A61K 45/06; C12N 2310/11; C12N 2310/315; C12N 2310/318; C12N 2310/321; C12N 2310/3231; C12N 2310/3341; C12N 2310/335; C12N 2310/341; C12N 2310/346; C12N 2310/3515

(57) ABSTRACT

Disclosed herein are antisense oligonucleotides, such as gapmer antisense oligonucleotides with a modified backbone chemistry (e.g., with one or more spacers). Such gapmer antisense oligonucleotides with modified backbone chemistries may be useful for treating various diseases, such as neurological diseases.

### CROSS-REFERENCE TO RELATED APPLICATIONS

This application claims the benefit of U.S. Provisional Application No. 63/285,705, filed on Dec. 3, 2021, U.S. Provisional Application No. 63/285,888, filed on Dec. 3, 2021, U.S. Provisional Application No. 63/285,692, filed on Dec. 3, 2021, U.S. Provisional Application No. 63/285,696, filed on Dec. 3, 2021, and U.S. Provisional Application No. 63/285,665, filed on Dec. 3, 2021, each of which is hereby incorporated herein by reference in their entireties for all purposes.

### REFERENCE TO A SEQUENCE LISTING XML

This application contains a sequence listing which has been submitted electronically in XML format. The Sequence Listing XML is incorporated herein by reference. Said XML file, created on Nov. 17, 2022, is named QRL-011WO_SL.xml and is 418,206,230 in size.

### BACKGROUND

Antisense oligonucleotides are nucleic acid-based compounds that can be used to inhibit expression of certain genes that are linked to diseases. Although antisense oligonucleotides can be generally designed to hybridize with target genes, conventional antisense oligonucleotides often exhibit poor efficacy. Thus, there is a need to develop modified antisense oligonucleotides that exhibit improved performance and efficacy for preventing, ameliorating, and treating diseases, examples of which include neurological diseases.

### SUMMARY

Disclosed herein is a compound comprising a gapmer oligonucleotide, and wherein the gapmer oligonucleotide comprises a spacer. Additionally disclosed herein is a gapmer oligonucleotide, wherein the gapmer oligonucleotide comprises a spacer. In various embodiments, the gapmer oligonucleotide comprises a second spacer that is non-adjacent to the spacer.

In various embodiments, the gapmer oligonucleotide comprises a sequence that is between 85 and 98% complementary to an equal length portion of a transcript whose presence leads to a neurological disease. In various embodiments, the gapmer oligonucleotide comprises a sequence that is between 85 and 98% complementary to an equal length portion of a PPM1A mRNA or pre-mRNA transcript, an ATXN2 mRNA or pre-mRNA transcript, a SOD1 mRNA or pre-mRNA transcript, or a MAPT mRNA or pre-mRNA transcript. In various embodiments, the gapmer oligonucleotide comprises a sequence that is between 85 and 98% complementary to an equal length portion of any one of SEQ ID NOs: 1909-1913, 149355-149361, 167802-167804, or 301567-301589, a sequence having 90% identity thereof, or to a 15 to 50 contiguous nucleobase portion thereof.

In various embodiments, the gapmer oligonucleotide comprises a sequence that shares at least 85% identity with an equal length portion of any one of SEQ ID NOs: 1-954, 1914-149354, 149362-158581, 167805-301566, SEQ ID NOs: 301692-301742 or the sequences in Tables 4A-4C. In various embodiments, the gapmer oligonucleotide comprises a sequence that shares at least 90% identity with an equal length portion of any one of SEQ ID NOs: 1-954, 1914-149354, 149362-158581, 167805-301566, SEQ ID NOs: 301692-301742 or the sequences in Tables 4A-4C. In various embodiments, the gapmer oligonucleotide comprises a sequence that shares at least 95% identity with an equal length portion of any one of SEQ ID NOs: 1-954, 1914-149354, 149362-158581, 167805-301566, SEQ ID NOs: 301692-301742 or the sequences in Tables 4A-4C. In various embodiments, the gapmer oligonucleotide comprises a sequence that shares at least 100% identity with an equal length portion of any one of SEQ ID NOs: 1-954, 1914-149354, 149362-158581, 167805-301566, SEQ ID NOs: 301692-301742 or the sequences in Tables 4A-4C.

In various embodiments, the gapmer oligonucleotide comprises a segment with at most 11 linked nucleosides, and wherein the gapmer oligonucleotide comprises a sequence that shares at least 85% identity with an equal length portion of any one of SEQ ID NOs: 1-954, 1914-149354, 149362-158581, 167805-301566, SEQ ID NOs: 301692-301742 or the sequences in Tables 4A-4C. In various embodiments, the gapmer oligonucleotide comprises a segment with at most 10, 9, 8, 7, 6, 5, 4, 3, or 2 linked nucleosides, and wherein the gapmer oligonucleotide comprises a sequence that shares at least 85% identity with an equal length portion of any one of SEQ ID NOs: 301692-301742 or the sequences in Tables 4A-4C. In various embodiments, the gapmer oligonucleotide comprises a segment with at most 11 linked nucleosides, and wherein the gapmer oligonucleotide comprises a sequence that shares at least 90% identity with an equal length portion of any one of SEQ ID NOs: 301692-301742 or the sequences in Tables 4A-4C. In various embodiments, the gapmer oligonucleotide comprises a segment with at most 10, 9, 8, 7, 6, 5, 4, 3, or 2 linked nucleosides, and wherein the gapmer oligonucleotide comprises a sequence that shares at least 90% identity with an equal length portion of any one of SEQ ID NOs: 301692-301742 or the sequences in Tables 4A-4C.

In various embodiments, the gapmer oligonucleotide is at least 18, at least 19, at least 20, at least 21, at least 22, at least 23, at least 24, or at least 25 oligonucleotide units in length. In various embodiments, at least one (i.e., one or more) nucleoside linkage of the gapmer oligonucleotide is a non-natural linkage, In various embodiments, the gapmer oligonucleotide is at least 19 oligonucleotide units in length.

In various embodiments, the spacer is a nucleoside-replacement group comprising a non-sugar substitute that is incapable of linking to a nucleotide base. In various embodiments, the spacer is located between positions 5 and 11 of the gapmer oligonucleotide. In various embodiments, the spacer is located between positions 7 and 11 of the gapmer oligonucleotide. In various embodiments, the gapmer oligonucleotide further comprises a second spacer, and wherein the spacer and the second spacer are non-adjacent to one another. In various embodiments, the gapmer oligonucleotide further comprises a second spacer, wherein the second spacer is located between positions 15 and 19 of the gapmer oligonucleotide. In various embodiments, the spacer and the second spacer are separated by at least 5 nucleobases, at least 6 nucleobases, or at least 7 nucleobases in the gapmer oligonucleotide. In various embodiments, the spacer is located between positions 5 and 11 of the gapmer oligonucleotide, and wherein the second spacer is located between positions 15 and 19 of the gapmer oligonucleotide. In various embodiments, the spacer is located at position 8 of the gapmer oligonucleotide, and wherein the second spacer is located at position 16 of the gapmer oligonucleotide. In various embodiments, the spacer is located at position 5 of the gapmer oligonucleotide, and wherein the second spacer is located at position 17 of the gapmer oligonucleotide. In various embodiments, the spacer is located at position 7 of the gapmer oligonucleotide, and wherein the second spacer is located at position 15 of the gapmer oligonucleotide. In various embodiments, the spacer is located at position 11 of the gapmer oligonucleotide, and wherein the second spacer is located at position 19 of the gapmer oligonucleotide.

In various embodiments, each of the spacer or second spacer is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base. In various embodiments, each of the spacer or second spacer is independently represented by Formula (X), wherein:

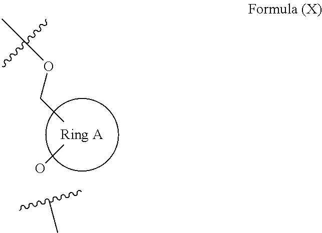

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the

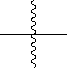

symbol represents the point of connection to an internucleoside linkage.

In various embodiments, each of the spacer or second spacer is independently represented by Formula (Xa), wherein:

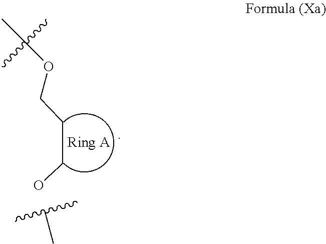

In various embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrrolidinyl, piperidinyl, piperazinyl, morpholinyl and azepanyl. In various embodiments, ring A is tetrahydrofuranyl. In various embodiments, ring A is tetrahydropyranyl.

In various embodiments, each of the spacer and second spacer is independently represented by Formula I, wherein:

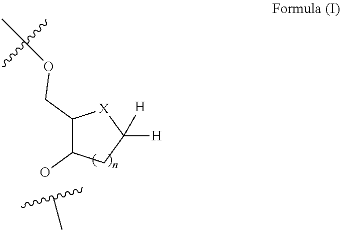

X is selected from —CH₂— and —O—; and
n is 0, 1, 2 or 3.

In various embodiments, each of the spacer and second spacer is independently represented by Formula I′, wherein:

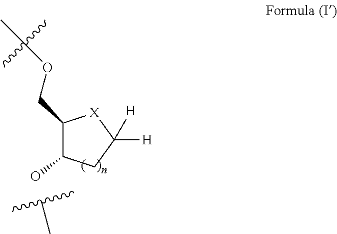

X is selected from —CH₂— and —O—; and
n is 0, 1, 2 or 3.

In various embodiments, each of the spacer and second spacer is independently represented by Formula (Ia), wherein:

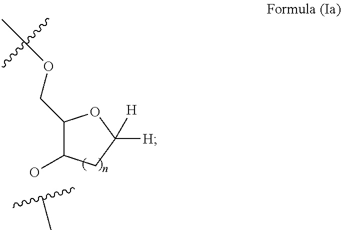

and
n is 0, 1, 2 or 3.

In various embodiments, each of the spacer and second spacer is independently represented by Formula (Ia′), wherein:

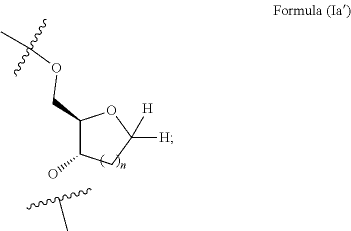

and
n is 0, 1, 2 or 3.

In various embodiments, each of the spacer and second spacer is independently represented by Formula II, wherein:

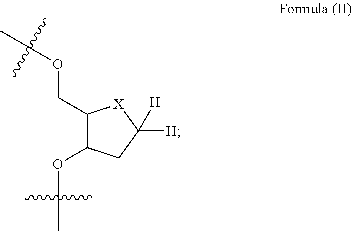

and
X is selected from —CH₂— and —O—.

In various embodiments, each of the spacer and second spacer is independently represented by Formula II′, wherein:

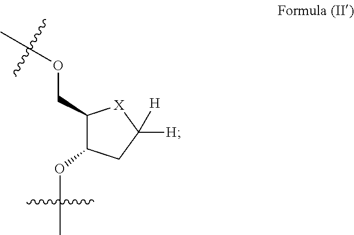

and
X is selected from —CH₂— and —O—.

In various embodiments, each of the spacer and second spacer is independently represented by Formula (Iia), wherein:

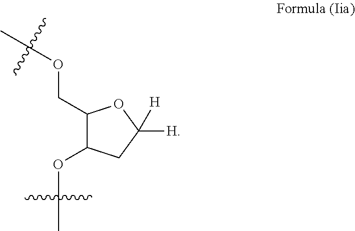

In various embodiments, each of the spacer and second spacer is independently represented by Formula (Iia′), wherein:

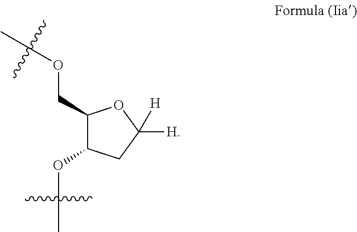

In various embodiments, each of the spacer and second spacer is independently is independently represented by Formula III, wherein:

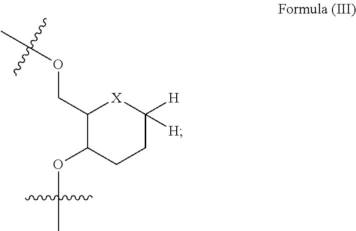

and
X is selected from —CH₂— and —O—.

In various embodiments, each of the spacer and second spacer is independently represented by Formula III′, wherein:

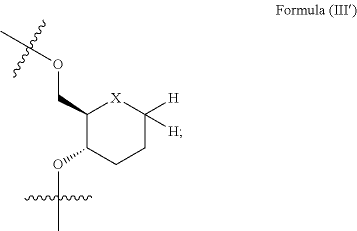

and
X is selected from —CH₂— and —O—.

In various embodiments, each of the spacer and second spacer is independently represented by Formula (IIIa), wherein:

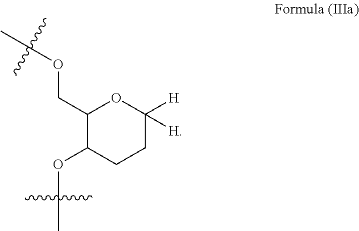

In various embodiments, each of the spacer and second spacer is independently represented by Formula (IIIa′), wherein:

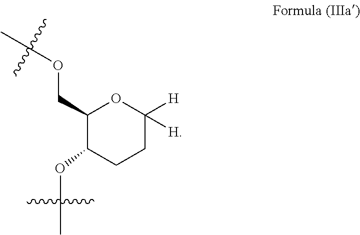

In various embodiments, the gapmer oligonucleotide comprising the spacer has a GC content of at least 10%. In various embodiments, the gapmer oligonucleotide comprising the spacer has a GC content of at least 20%. In various embodiments, the gapmer oligonucleotide comprising the spacer has a GC content of at least 25%. In various embodiments, the gapmer oligonucleotide comprising the spacer has a GC content of at least 30%. In various embodiments, the gapmer oligonucleotide comprising the spacer has a GC content of at least 40%. In various embodiments, the gapmer oligonucleotide comprising the spacer has a GC content of at least 50%. In various embodiments, at least one (i.e., one or more) nucleoside linkage of the gapmer oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In various embodiments, at least one internucleoside linkage of the nucleotide sequence is a phosphorothioate linkage. In various embodiments, the phosphorothioate internucleoside linkage is in one of a Rp configuration or a Sp configuration.

In various embodiments, the gapmer oligonucleotide comprises one or more chiral centers and/or double bonds. In various embodiments, the gapmer oligonucleotide exists as stereoisomers selected from geometric isomers, enantiomers, and diastereomers. In various embodiments, all internucleoside linkages of the nucleotide sequence are phosphorothioate linkages. In various embodiments, the gapmer oligonucleotide comprises at least one modified nucleobase. In various embodiments, the at least one modified nucleobase is 5-methylcytosine, pseudouridine, or 5-methoxyuridine. In various embodiments, the gapmer oligonucleotide comprises at least one nucleoside with a modified sugar moiety. In various embodiments, the modified sugar moiety is one of a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

In various embodiments, the gapmer oligonucleotide comprises two, three, four, five, six, seven, eight, nine, or ten nucleosides with modified sugar moieties. In various embodiments, the modified sugar moieties are independently any one of a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA). In various embodiments, the gapmer oligonucleotide comprises ten 2′-O-(2-methoxyethyl) (2′-MOE) nucleosides. In various embodiments, five of the 2′-O-(2-methoxyethyl) (2′-MOE) nucleosides are located at the 3′ end of the gapmer oligonucleotide, and wherein five of the 2′-O-(2-methoxyethyl) (2′-MOE) nucleosides are located at the 5′ end of the gapmer oligonucleotide. In various embodiments, the at least one nucleoside with the modified sugar moiety or the nucleosides with modified sugar moieties are ribonucleosides. In various embodiments, the gapmer oligonucleotide comprises at least one deoxyribonucleoside. In various embodiments, the gapmer oligonucleotide comprises two, three, four, five, six, seven, eight, nine, or ten deoxyribonucleosides.

In various embodiments, the gapmer oligonucleotide comprises: a gap segment comprising one or more of linked deoxyribonucleosides, 2′-Fluoro Arabino Nucleic Acids (FANA), and Fluoro Cyclohexenyl nucleic acid (F-CeNA); a 5′ wing region comprising linked nucleosides; and a 3′ wing region comprising linked nucleosides; wherein the central region comprises a region of at least 8 oligonucleotide units comprising at least 4 contiguous nucleobases, the region having at least 80% identity to an equal length portion of any one of SEQ ID NOs: 1-954, 1914-149354, 149362-158581, 167805-301566, SEQ ID NOs: 301692-301742 or the sequences in Tables 4A-4C positioned between the 5′ wing segment and the 3′ wing segment; wherein the 5′ wing region and the 3′ wing region each comprises at least two linked nucleosides; and wherein at least one nucleoside of each wing region comprises a modified sugar.

In various embodiments, the at least two linked nucleosides of the 5′ wing region are linked through a phosphorothioate internucleoside linkage and/or wherein the at least two linked nucleosides of the 3′ wing region are independently linked through a phosphorothioate internucleoside linkage. In various embodiments, every internucleoside linkage of the 5′ wing region and/or every internucleoside linkage of the 3′ wing region, independently are phosphorothioate internucleoside linkages. In various embodiments, the 5′ wing region further comprises at least one phosphodiester internucleoside linkage. In various embodiments, the 3′ wing region further comprises at least one phosphodiester internucleoside linkage. In various embodiments, the at least two linked nucleosides of the 5′ wing region are linked through a phosphodiester internucleoside linkage and/or wherein the at least two linked nucleosides of the 3′ wing region are independently linked through a phosphodiester internucleoside linkage. In various embodiments, at least one of the internucleoside linkages of the central region is a phosphodiester linkage. In various embodiments, at least two, at least three, at least four, at least five, at least six, at least seven, at least eight, or at least nine of the internucleoside linkages of the central region are phosphodiester linkages. In various embodiments, at least one of the internucleoside linkages of the central region is a phosphorothioate internucleoside linkage. In various embodiments, at least two, at least three, at least four, at least five, at least six, at least seven, at least eight, or at least nine of the internucleoside linkages of the central region are phosphorothioate internucleoside linkages. In various embodiments, all internucleoside linkages of the gapmer oligonucleotide are phosphorothioate internucleoside linkages. In various embodiments, any one or all of the phosphorothioate internucleoside linkages are in a Rp configuration, a Sp configuration, or in any combination of Rp and Sp configuration.

In various embodiments, the gapmer oligonucleotide comprises at least one modified sugar moiety. In various embodiments, the 5′ wing region or the 3′ wing region comprises the at least one modified sugar moiety. In various embodiments, the central region comprises the at least one modified sugar moiety. In various embodiments, the at least one modified sugar moiety is any one of a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA). In various embodiments, the gapmer oligonucleotide comprises one or more 2′-MOE nucleosides. In various embodiments, the 5′ wing region or the 3′ wing region comprise one or more 2′-MOE nucleosides. In various embodiments, the 5′ wing region or the 3′ wing region comprise two, three, four, five, or six 2′-MOE nucleosides. In various embodiments, every nucleoside of the 5′ wing region or the 3′ wing region is a 2′-MOE nucleoside. In various embodiments, the central region comprises one or more 2′-MOE nucleosides. In various embodiments, the central region comprises two, three, four, five, six, seven, eight, nine, or ten 2′-MOE nucleosides. In various embodiments, every nucleoside of the central region is a 2′-MOE nucleoside. In various embodiments, the one or more 2′-MOE nucleosides are linked through phosphorothioate internucleoside linkages.

In various embodiments, the gapmer oligonucleotide comprises sugar modifications in any of the following patterns: eeeee-d10-eeeee, eeeee-d8-eeeee, eeeeee-d11-eeeeee, eee-d8-eee, eee-d10-eee, eeee-d10-eeee, and eeee-d8-eeee, wherein e=2′-MOE nucleoside and d=a deoxyribonucleoside, and wherein at least one “e” or at least one “d” is replaced with a spacer. In various embodiments, the gapmer oligonucleotide comprises internucleoside linkages in any of the following patterns: sssssooooooooosssss; ooooosssssssssooooo; oooooooooooooosssss; soossssssssssssssss; sososssssssssssosos; ssssssssssssssssoos; sssssoooooooooooooo; sssssssssssssssssss; ssssssssssssssssssssss; sssooooooosss; ooosssssssooo; sssssssssssss; sosssssssssos; sosssssssssss; sssssssssssos; ssssssssssooo; ooossssssssss; sssooooooooosss; ooosssssssssooo; sssssssssssssss; ssssssssssssooo; ooossssssssssss; sosssssssssssos; sosssssssssssss; sssssssssssssos; ssssooooooooossss; oooosssssssssoooo; sssssssssssssssss; sssssssssssssoooo; soosssssssssssoos; soossssssssssssss; ssssssssssssssoos; oooosssssssssssss; ssssooooooossss; oooosssssssoooo; sssssssssssoooo; oooosssssssssss; soosssssssssoos; soossssssssssss; ssssssssssssoos; soooossssssssssooos; soooossssssssssooss; sossssssssssssoss; sosssssssssssssosss; or sssssssssssssss; wherein s=a phosphorothioate linkage, and o=a phosphodiester linkage.

In various embodiments, the gapmer oligonucleotide comprises sugar modification and internucleoside linkage combinations, respectively, in any of the following patterns:
a) eeeee-d10-eeeee and sssssooooooooosssss;
b) eeeee-d10-eeeee and ooooosssssssssooooo;
c) eeeee-d10-eeeee and sssssssssssssssssss;
d) eee-d8-eee and sssooooooosss;
e) eee-d8-eee and ooosssssssooo
f) eee-d8-eee and sssssssssssss;
g) eee-d10-eee and sssooooooooosss;
h) eee-d10-eee and ooosssssssssooo;
i) eee-d10-eee and sssssssssssssss;
j) eeee-d10-eeee and ssssooooooooossss;
k) eeee-d10-eeee and oooosssssssssoooo;
l) eeee-d10-eeee and sssssssssssssssss;
m) eeee-d8-eeee and ssssooooooossss,
n) eeee-d8-eeee and oooosssssssoooo,
o) eeee-d8-eeee and sssssssssssssss,
p) eeeeee-d11-eeeeee and ssssssssssssssssssssss;
q) eeeee-d10-eeeee and sososssssssssssosos;
r) eeeee-d10-eeeee and soooossssssssssooos;
s) eeeee-d10-eeeee and soooossssssssssooss;
t) eeeee-d8-eeeee and sossssssssssssoss;
u) eeeee-d10-eeeee and sosssssssssssssosss;
v) eeeeee-d10-eeee and sssssssssssssssssss;
wherein e=2′-MOE nucleoside and d=a deoxyribonucleoside, and wherein s=a phosphorothioate linkage, and o=a phosphodiester linkage, and wherein at least one “e” or at least one “d” of the gapmer oligonucleotide is replaced with a spacer.

In various embodiments, the gapmer oligonucleotide comprises at least one modified nucleobase. In various embodiments, the 5′ wing region or the 3′ wing region comprises the at least one modified nucleobase. In various embodiments, the central region comprises the at least one modified nucleobase. In various embodiments, the at least one modified nucleobase is 5-methylcytosine, pseudouridine, or 5-methoxyuridine. In various embodiments, every cytosine in the 5′ wing region or the 3′ wing region is a 5-methylcytosine. In various embodiments, every cytosine in the central region is a 5-methylcytosine. In various embodiments, the gapmer oligonucleotide comprises sugar modification and internucleoside linkage combination of eeeee-d10-eeeee and sssssssssssssssssss, wherein e=2′-MOE nucleoside and d=a deoxyribonucleoside, wherein s=a phosphorothioate linkage, wherein at least one “e” or at least one “d” of the gapmer oligonucleotide is replaced with a spacer, and wherein each cytosine of the 2′-MOE nucleosides is a 5-methylcytosine.

In various embodiments, the gapmer oligonucleotide further comprises a conjugate moiety. In various embodiments, the conjugate moiety is a cholesterol conjugate located on the 3′ end of the gapmer oligonucleotide.

Additionally disclosed herein is a pharmaceutical composition comprising a gapmer oligonucleotide disclosed herein, or a pharmaceutically acceptable salt thereof, and a pharmaceutically acceptable excipient.

Additionally disclosed herein is a method of treating a neurological disease in a patient in need thereof, the method comprising administering to the patient a gapmer oligonucleotide disclosed herein or a pharmaceutically acceptable salt thereof, or a pharmaceutical composition disclosed herein. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

In various embodiments, the gapmer oligonucleotide is administered topically, parenterally, intrathecally, orally, pulmonarily, intratracheally, intranasally, transdermally, buccally, intrathalamically, intracerebroventricularly, intraocularly, sublingually, rectally, vaginally, or intraduodenally. In various embodiments, the gapmer oligonucleotide is administered intrathecally. In various embodiments, a therapeutically effective amount of the gapmer oligonucleotide is administered. In various embodiments, the patient is a human. In various embodiments, the pharmaceutical composition is suitable for topical, parenteral, intrathecal, oral, pulmonary, intratracheal, intranasal, transdermal, buccal, intrathalamical, intracerebroventricular, intraocular, sublingual, rectal, vaginal, or intraduodenal.

Additionally disclosed herein is use of a gapmer oligonucleotide in the manufacture of a medicament for the treatment of neurological disease. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism. In various embodiments, the gapmer oligonucleotide is a gapmer oligonucleotide disclosed herein.

Additionally disclosed herein is a method of treating a neurological disease in a patient in need thereof, the method comprising administering to a patient in need thereof a therapeutically effective amount of a pharmaceutical composition comprising a gapmer oligonucleotide, and a pharmaceutically acceptable excipient. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism. In various embodiments, the gapmer oligonucleotide is the gapmer oligonucleotide disclosed herein, a pharmaceutically acceptable salt thereof, or a pharmaceutical composition disclosed herein. In various embodiments, the pharmaceutical composition is administered topically, parenterally, orally, pulmonarily, rectally, buccally, sublingually, vaginally, intratracheally, intranasally, intrathecally, intracisternally, transdermally, or intraduodenally. In various embodiments, the pharmaceutical composition is administered intrathecally. In various embodiments, the patient is human.

Additionally disclosed herein is a gapmer oligonucleotide, or a pharmaceutically acceptable salt thereof, for use as a medicament. Additionally disclosed herein is a gapmer oligonucleotide, or a pharmaceutically acceptable salt thereof, for use in the treatment of a neurological disease. In various embodiments, said neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

Additionally disclosed herein is a gapmer oligonucleotide comprising the sequence of any one of SEQ ID NOs: 301692-301742 or the sequences in Tables 4A-4C, or a pharmaceutically acceptable salt thereof; wherein the gapmer oligonucleotide further comprises: a gap segment comprising one or more of linked deoxyribonucleosides, 2′-Fluoro Arabino Nucleic Acids (FANA), and Fluoro Cyclohexenyl nucleic acid (F-CeNA); a 5′ wing region comprising linked nucleosides; and a 3′ wing region comprising linked nucleosides; wherein the gapmer oligonucleotide comprises sugar modifications of eeeee-d10-eeeee, wherein e=2′-MOE nucleoside and d=a deoxyribonucleoside, wherein at least one “e” or at least one “d” is replaced with a spacer, and wherein each cytosine of the 2′MOE nucleosides is a 5-methylcytosine.

In various embodiments, at least one internucleoside linkage of the gapmer oligonucleotide is a phosphorothioate linkage. In various embodiments, the phosphorothioate internucleoside linkage is in one of a Rp configuration or a Sp configuration. In various embodiments, all internucleoside linkages of the gapmer oligonucleotide are phosphorothioate linkages. In various embodiments, the gapmer oligonucleotide comprises sugar modification and internucleoside linkage combinations, respectively, in any of the following patterns:
a) eeeee-d10-eeeee and sssssooooooooosssss;
b) eeeee-d10-eeeee and ooooosssssssssooooo;
c) eeeee-d10-eeeee and sssssssssssssssssss;
d) eee-d8-eee and sssooooooosss;
e) eee-d8-eee and ooosssssssooo
f) eee-d8-eee and sssssssssssss;
g) eee-d10-eee and sssooooooooosss;
h) eee-d10-eee and ooosssssssssooo;
i) eee-d10-eee and sssssssssssssss;
j) eeee-d10-eeee and ssssooooooooossss;
k) eeee-d10-eeee and oooosssssssssoooo;
l) eeee-d10-eeee and sssssssssssssssss;
m) eeee-d8-eeee and ssssooooooossss,
n) eeee-d8-eeee and oooosssssssoooo,
o) eeee-d8-eeee and sssssssssssssss,
p) eeeeee-d11-eeeeee and ssssssssssssssssssssss;
q) eeeee-d10-eeeee and sososssssssssssosos;
r) eeeee-d10-eeeee and soooossssssssssooos;
s) eeeee-d10-eeeee and soooossssssssssooss;
t) eeeee-d8-eeeee and sossssssssssssoss;
u) eeeee-d10-eeeee and sosssssssssssssosss,
wherein at least one “e” or at least one “d” of the gapmer oligonucleotide is replaced with a spacer.

Additionally disclosed herein is a pharmaceutical composition comprising an antisense gapmer oligonucleotide disclosed herein, or a pharmaceutically acceptable salt thereof, and a pharmaceutically acceptable excipient. In various embodiments, the spacer is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base. In various embodiments, the spacer is represented by Formula (X), wherein:

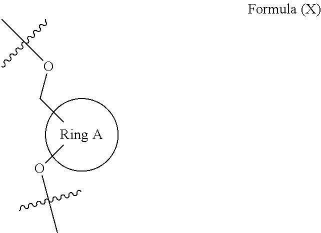

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the

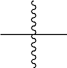

symbol represents the point of connection to an internucleoside linkage.

In various embodiments, the spacer is represented by Formula (Xa), wherein:

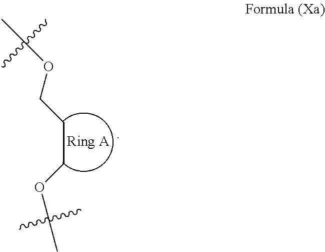 [SMILES: *OCCCO*]

In various embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl. In various embodiments, ring A is tetrahydrofuranyl. In various embodiments, ring A is tetrahydropyranyl.

In various embodiments, the spacer is represented by Formula (I), wherein:

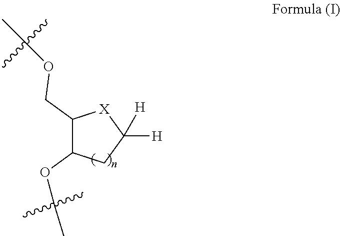

X is selected from —CH₂— and —O—; and
n is 0, 1, 2 or 3.

In various embodiments, the spacer is represented by Formula (I′), wherein:

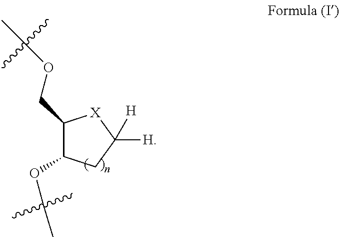

In various embodiments, the spacer is represented by Formula (Ia), wherein:

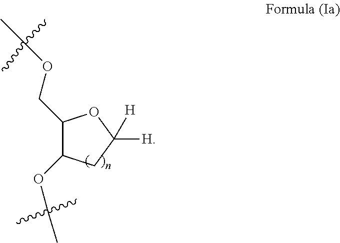

In various embodiments, the spacer is represented by Formula (Ia′), wherein:

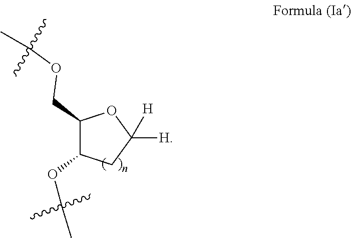

In various embodiments, the spacer is represented by Formula II, wherein:

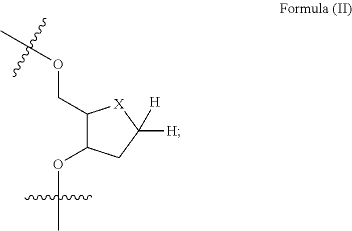

and
X is selected from —CH₂— and —O—.

In various embodiments, the spacer is represented by Formula II′, wherein:

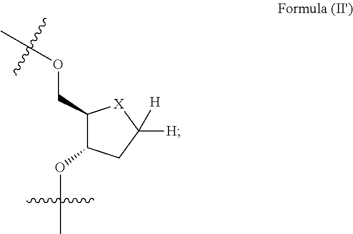

and
X is selected from —CH₂— and —O—.

In various embodiments, the spacer is represented by Formula (Iia), wherein:

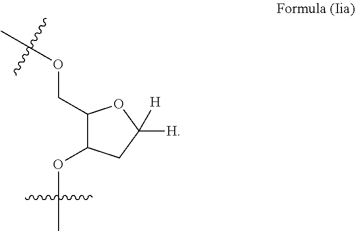

In various embodiments, the spacer is represented by Formula (Iia′), wherein:

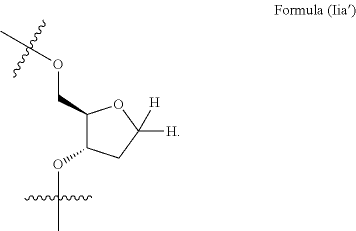

In various embodiments, the spacer is represented by Formula III, wherein:

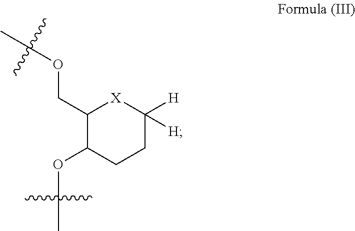

and
X is selected from —CH₂— and —O—.

In various embodiments, the spacer is represented by Formula III′, wherein:

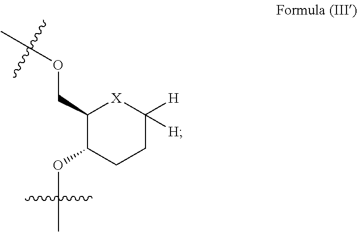

and
X is selected from —CH₂— and —O—.

In various embodiments, the spacer is represented by Formula (IIIa), wherein:

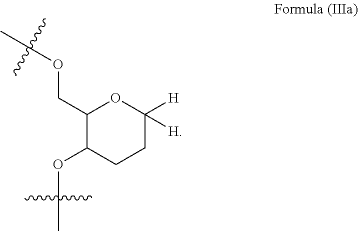

In various embodiments, the spacer is represented by Formula (IIIa′), wherein:

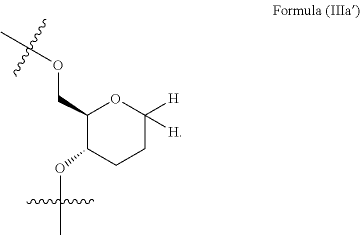

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising Riluzole (Rilutek), troriluzole, Edaravone (Radicava), rivastigmine, donepezil, QRL-101. QRL-201, galantamine, selective serotonin reuptake inhibitor, antipsychotic agents, cholinesterase inhibitors, memantine, benzodiazepine antianxiety drugs, AMX0035 (ELYBRIO®), ZILUCOPLAN (RA101495), dual AON intrathecal administration (e.g., BIIB067, BIIB078), BIIB100, levodopa/carbidopa, dopaminergic agents (e.g., ropinirole, pramipexole, rotigotine), medroxyprogesterone, KCNQ2/KCNQ3 openers, Pridopidine, PrimeC (combination of ciprofloxacin and Celebrex), olanzapine (Zyprexa), quetiapine (Seroquel), SSRIs, divalproex sodium (Depakote), carbamazepine (Tegretol), medroxyprogestrone, lithium, anticonvulsants and psychostimulant agents, breathing care, physical therapy, occupational therapy, speech therapy, nutritional support, or any combination thereof. In various embodiments, the neurological disease is any one of amyotrophic lateral sclerosis (ALS), frontotemporal dementia (FTD), or ALS with FTD.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising Memantine, Rivastigmine, Galantamine, Donepezil, QRL-101, QRL-201, Aricept®, Exelon® (Rivastigmine), Razadyne®, Aducanumab, BAN2401, BIIB091 (gosuranemab), BIIB076, BIIB080 (IONIS-MAPTRx), Elayta (CT1812), MK1942, allogenic hMSC, nilotinib, ABT-957, acitretin, ABT-354, GV1001, Riluzole, CAD106, CNP520, AD-35, Rilapladib, DHP1401, T-817 MA, TC-5619, TPI-287, RVT-101, LY450139, JNJ-54861911, Dapagliflozin, GSK239512, PF-04360365, ASP0777, SB-742457 (a 5-HT6 receptor antagonist), PF-03654746 (an H₃ receptor antagonist), GSK933776 (an Fc-inactivated anti-β amyloid (Aβ) monoclonal antibody (mAb)), Posiphen ((+)-phenserine tartrate), AMX0035 (ELYBRIO®), coenzyme Q10, aducanamab (ADUHLEM), memantine (NAMENDA), Namzeric, Suvorexant (belsomra), lecanemab, or any combination thereof. In various embodiments, the neurological disease is Alzheimer's Disease.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising Levodopa, Carbidopa-levidopa, pramipexole (MIRAPEX), ropinirole (REQUIP), rotigotine (NEUPRO), apomorphine (APOKYN, KYNMOBI), selegiline (EDLEPRYL, ZELAPAR), rasagiline, entacapone (COMTAN), tolcapone (TASMAR), amantadine (GOCOVRI, SYMMETREL, OSMOLEX), trihexyphenidyl (ARTANE), BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, istradefylline (NOURIANZ), safinamide (XADAGO), benztropine (COGENTIN), opicapone (ongentys), exenatide, lingzhi, Caffeine, sarizotan, embryonic dopamine cell implantation, or any combination thereof. In various embodiments, the neurological disease is Parkinson's Disease.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising UCB0107, ABBV-8E12, F-18 AV1451, BIII1B092, C2N-8E12, tideglusib, deep transcranial magnetic stimulation, lipoic acid, tolfenamica acid, lithium, AZP2006, Glial Clell Line-Derived Neurotrophic Factor, NBMI, suvorxant, zolpidem, TPI 287, davunetide, pimavanserin, Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof. In various embodiments, the neurological disease is progressive supranuclear palsy (PSP).

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising Tetrabenazine, deutetrabenazine, physical therapy, risperidone, haloperidol, chlorpromazine, clonazepam, diazepam, benzodiazepines, selective serotonin reuptake inhibitors, quetiapine, carbatrol, valproate, lamotrigine, pridopidine, delta-9-tetrahydrocannabinol, cannabidiol, stem-cell therapy, ISIS-443139, nilotinib, resveratrol, neflamapimod, fenofibrate, creatine, RO7234292, SAGE-718, WVE-120102, WVE-120101, dimebon, minocycline, deep brain stimulation, ursodiol, coenzyme Q10, OMS643762, VX15/2503, PF-02545920, BN82451B, SEN0014196, olanzapine, tiapridal (tiapride), or any combination thereof. In various embodiments, the neurological disease is Huntington's Disease.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising anticoagulants, antidepressants, muscle relaxants, stimulants, anticonvulsants, anti-anxiety medication, erythropoietin, hyperbaric treatment, rehabilitation therapies (e.g., physical, occupational, speech, psychological, or vocational counseling), or any combination thereof. In various embodiments, the neurological disease is brain trauma.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising AXER-204, glyburide, 5-hydroxytryptophan (5-HTP), L-3,4-dihydroxyphenylalanine (L-DOPA), or rehabilitation therapies (e.g., physical therapy, occupational therapy, recreational therapy, use of assistive devices, improved strategies for exercise and healthy diets), or any combination thereof. In various embodiments, the neurological disease is spinal cord injury.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising TPI-287, lithium, occupational, physical, and speech therapy, or any combination thereof can be selected as an additional therapy. In various embodiments, the neurological disease is corticobasal degeneration.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising gabapentin, pregabalin, lamotrigine, carbamazepine, duloxetine, gabapentinoids, tricyclic antidepressants, serotonin-norepinephrine reuptake inhibitors, opioids, neurotoxin, dextromethorphan, nicotinamide riboside, auto-antibodies targeting neuronal antigens (TS-HDS and FGFR3), or any combination thereof. In various embodiments, the neuropathy is a chemotherapy induced neuropathy.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising amantadine, armodafinil, baclofen, buspirone, carbamazepine, citalopram, clonazepam, desvenlafaxine, diazepam, duloxetine, escitalopram, flunarizine, fluoxetine, fluvoxamine, gabapentin, isoniazid, levetiracetam, levodopa, memantine, modafinil, ondansetron, paroxetine, pramipexole, primidone, riluzole, ropinirole, sertraline, tizanidine, topiramate, trihexyphenidyl, valproic acid, venlafaxine, BHV-4157, or a combination thereof. In various embodiments, the neurological disease is spinocerebellar ataxia.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), zonisamide (zonegran), or any combination thereof. In various embodiments, the neurological disease is epilepsy.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising nusinersen (SPINRAZA), onasemnogene abeparvovec-xioi (ZOLGENSMA), risdiplam (EVRYSDI), or any combination thereof. In various embodiments, the neurological disease is spinal muscular atrophy.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising anti-seizure medications, speech therapy, physical therapy, occupational therapy, Adrabetadex, Arimoclomol, N-Acetyl-L-Leucine, or any combination thereof. In various embodiments, the neurological disease is Niemann-Pick disease type C.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising physical and occupational therapies, orthopedic surgery, orthopedic devices, PXT3003, or any combination thereof. In various embodiments, the neurological disease is Charcot-Marie-Tooth Disease (CMT).

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising enzyme replacement therapy: idursulfase (Elaprase), surgical intervention (tonsillectomy and/or adenoidectomy), RGX-121 gene therapy, adalimumab, MT2013-31, or any combination thereof. In various embodiments, the neurological disease is Mucopolysaccharidosis type II (MPSIIA).

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising physical, occupational, and speech therapies, contact lenses and artificial tears, genetic counseling, or any combination thereof. In various embodiments, the neurological disease is Mucolipidosis IV.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising anticonvulsants, physical and occupational therapies, galactosidase, gene delivery of galactosidase, LYS-GM101 gene therapy, or any combination thereof. In various embodiments, the neurological disease is GM1 gangliosidosis.

Additionally disclosed herein is a method of treating a neurological disease and/or a neuropathy in a patient in need thereof, the method comprising administering to a patient in need thereof a therapeutically effective amount of a gapmer oligonucleotide, a pharmaceutically acceptable salt thereof, or a pharmaceutical composition, in combination with a second therapeutic agent selected from a group comprising physical and occupational therapies, use of devices such as braces, walkers, wheelchairs, immunosuppressants, BYM338, or any combination thereof. In various embodiments, the neurological disease is Sporadic inclusion body myositis (sIBM).

Additionally disclosed herein is a method of treating a neurological disease and/or a neuropathy in a patient in need thereof, the method comprising administering to a patient in need thereof a therapeutically effective amount of a gapmer, a pharmaceutically acceptable salt thereof, or a pharmaceutical composition, in combination with a second therapeutic agent selected from a group comprising corticosteroids, colchicine, dapsone, azathioprine, or any combination thereof. In various embodiments, the neurological disease is Henoch-Schonlein purpura (HSP).

Additionally disclosed herein is a method of treating a neurological disease and/or a neuropathy in a patient in need thereof, the method comprising administering to a patient in need thereof a therapeutically effective amount of a gapmer oligonucleotide, or a pharmaceutically acceptable salt thereof, or a pharmaceutical composition, in combination with a second therapeutic agent selected from a group comprising enzyme replacement therapy, substrate reduction therapy, N-acetylcysteine, GZ/SAR402671, cerezyme, or any combination thereof. In various embodiments, the neurological disease is Gaucher's disease.

### BRIEF DESCRIPTION OF THE DRAWINGS

FIG. 1 shows an example antisense oligonucleotide (AON), a portion of which is complementary to a mRNA transcript or pre-mRNA transcript. Dashed lines indicate positions of the AON which may or may not be complementary to corresponding positions of the transcript.

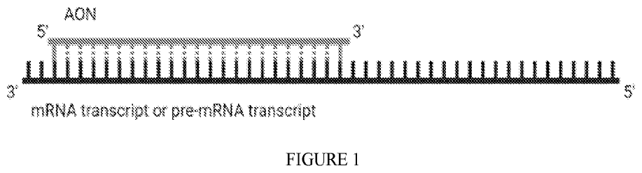

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound, for example, an antisense oligonucleotide (AON), as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or non-human primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like). In some embodiments, the mammal treated in the methods of the invention is desirably a mammal in whom modulation of a target expression and/or activity is desired.

As used herein, “PPM1A” (also known as Protein Phosphatase, Mg²⁺/Mn²⁺ Dependent 1A, Protein Phosphatase 1A (Formerly 2C), Magnesium-Dependent, Alpha Isoform, Protein Phosphatase TA, EC 3.1.3.16, Protein Phosphatase 2C Isoform Alpha, Protein Phosphatase IA, Phosphatase 2C Alpha, PP2C-Alpha, PPPM1A, and PP2CA) refers to the gene or gene products (e.g., protein or mRNA transcript (including pre-mRNA) encoded by the gene) identified by Entrez Gene ID No. 5494 and allelic variants thereof, as well as orthologs found in non-human species (e.g., non-human primates or mice).

As used herein, “ATXN2” (also known as Ataxin 2, ATX2, TNRC13, SCA2, Trinucleotide Repeat-Containing Gene 13 Protein, Spinocerebellar Ataxia Type 2 Protein, Ataxin-2, Spincocerebellar Ataxia 2 (Olivopontocerebellar Ataxia 2, Autosomal Dominant, Ataxin 2), and Trinucleotide Repeat Containing 13) refers to the gene or gene products (e.g., protein or mRNA transcript (including pre-mRNA) encoded by the gene) identified by Entrez Gene ID No. 6311 and allelic variants thereof, as well as orthologs found in non-human species (e.g., non-human primates or mice).

As used herein, “SOD1” (also known as Superoxide Dismutase 1, IPOA, Superoxide Dismutase 1, Soluble, Superoxide Dismutase [Cu—Zn], EC 1.15.1.1, HSod1, ALS1, ALS, Amyotrophic Lateral Sclerosis 1 (Adult), Epididymis Secretory Protein Li 44, Superoxide Dismutase, Cystolic, Cu/Zn Superoxide Dismutase, Indophenoloxidase A, SOD, Soluble, Homodimer, HEL-S-44, STAHP, and SOD) refers to the gene or gene products (e.g., protein or mRNA transcript (including pre-mRNA) encoded by the gene) identified by Entrez Gene ID No. 6647 and allelic variants thereof, as well as orthologs found in non-human species (e.g., non-human primates or mice).

As used herein, “MAPT” (also known as Microtubule Associated Protein Tau, MTBT1, PPPTR103, FTDP-17, MTBT2, MAPTL, PPND, MSTD, TAU, G Protein Beta1/Gamma2, Subunit-Interacting Factor, Protein Phosphatase 1, Regulatory Subunit 103, Microtubule-Associated Protein Tau, Neurofibrillary Tangle Protein, Paired Helical Filament-Tau, MGC138549, FLJ31424, PHF-Tau, Tau-40, DDPAC, Tau, and Microtubule-Associated Protein Tau, Isoform 4) refers to the gene or gene products (e.g., protein or mRNA transcript (including pre-mRNA) encoded by the gene) identified by Entrez Gene ID No. 4137 and allelic variants thereof, as well as orthologs found in non-human species (e.g., non-human primates or mice).

In the present specification, the term “therapeutically effective amount” means the amount of the subject AON that will elicit the biological or medical response of a tissue, system, animal or human that is being sought by the researcher, veterinarian, medical doctor, or other clinician. The AONs of the invention are administered in therapeutically effective amounts to treat and/or prevent a disease, condition, disorder, or state, for example, ALS, FTD, ALS with FTD, or another motor neuron disease or neurological disease or condition. Alternatively, a therapeutically effective amount of an AON is the quantity required to achieve a desired therapeutic and/or prophylactic effect, such as an amount which results in the prevention of or a decrease in the symptoms associated with a disease disclosed herein.

As used herein, the term “antisense oligonucleotide” or “AON” encompasses antisense oligonucleotides that target genes or gene products of any of PPM1A, ATXN2, SOD1, or MAPT. “Antisense oligonucleotide” or “AON” encompass any of a parent oligonucleotide, an oligonucleotide variant, an oligonucleotide with one or more spacers, an oligonucleotide variant with one or more spacers, a gapmer antisense oligonucleotide (AON), and a gapmer AON with one or more spacers. Examples of antisense oligonucleotides include oligonucleotides comprising a sequence of any one of antisense oligonucleotide comprising any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C.

The terms “parent oligonucleotide,” “parent antisense oligonucleotide,” or “parent AON” refer to an antisense oligonucleotide that is complementary to a portion of a target gene product, such as any one of a PPM1A, ATXN2, SOD1, or MAPT mRNA or pre-mRNA transcript. Parent oligonucleotides do not include a spacer. In particular embodiments, parent oligonucleotides include 20 linked nucleosides. In such embodiments, parent oligonucleotides are 20mers. Examples of parent oligonucleotides are AONs with a sequence of any one of SEQ ID NOs: 1-954, 1914-149354, 149362-158581, or 167805-301566. As described hereafter, oligonucleotide with spacers, oligonucleotide variants, and gapmer AONs are described in relation to a corresponding parent oligonucleotide.

The term “oligonucleotide variant” refers to an antisense oligonucleotide that represents a modified version of a corresponding parent oligonucleotide. For example, an oligonucleotide variant represents a shortened version of a parent oligonucleotide. In various embodiments, an oligonucleotide variant is any one of a 15mer, 16mer, 17mer, 18mer 19mer, 20mer, 21mer, 22mer or 23mer. For example, an AON variant may be a shorter or longer version of a corresponding parent oligonucleotide that comprises a nucleobase sequence selected from any one of SEQ ID NOs: 1-954, 1914-149354, 149362-158581, or 167805-301566). Examples of oligonucleotide variants include oligonucleotides comprising a sequence of any one of SEQ ID NOs: 301590-301594. In various embodiments, oligonucleotide variants comprise one or more spacers.

The term “oligonucleotide with one or more spacers” or “oligonucleotide comprising a spacer” refers to an oligonucleotide with at least one spacer. An oligonucleotide with one or more spacers can, in various embodiments, include one spacer, two spacers, three spacers, four spacer, five spacers, six spacers, seven spacers, eight spacers, nine spacers, or ten spacers. In various embodiments, an oligonucleotide comprising one or more spacers includes at least one segment with at most 10 linked nucleosides. For example, as described in a 5′ to 3′ direction, an oligonucleotide comprising a spacer can include a segment with 10 linked nucleosides followed by a spacer, followed by a second segment of 9 linked nucleosides. In various embodiments, an oligonucleotide comprising one or more spacers includes at least one segment with at most 7 linked nucleosides. For example, as described in a 5′ to 3′ direction, an oligonucleotide comprising a spacer can include a segment with 7 linked nucleosides, followed by a spacer, a second segment with 9 linked nucleosides, followed by a second spacer, and a third segment with 7 linked nucleosides. Here, the first segment of 7 linked nucleosides and the third segment of 7 linked nucleosides each represents segments with 7 linked nucleosides. In various embodiments, an oligonucleotide comprising one or more spacers includes at least one segment with at most 6 linked nucleosides. For example, as described in a 5′ to 3′ direction, an oligonucleotide comprising a spacer can include a segment with 6 linked nucleosides, followed by a spacer, a second segment with 6 linked nucleosides, followed by a second spacer, and a third segment with 6 linked nucleosides. Here, the first segment of 6 linked nucleosides, the second segment of 6 linked nucleosides, and the third segment of 6 linked nucleosides each represents segments with 6 linked nucleosides. Here, every segment of an oligonucleotide with one or more spacers has at most 6 linked nucleosides In various embodiments, the gapmer oligonucleotide comprises a segment with at most 10, 9, 8, 7, 6, 5, 4, 3, or 2 linked nucleosides.

As another example, an oligonucleotide comprising a spacer can include a segment with 10 linked nucleosides, followed by a spacer, a second segment with 10 linked nucleosides, followed by a second spacer, and a third segment with 3 linked nucleosides. Here, the third segment of 3 linked nucleosides represents the segment with at most 6 or at most 7 linked nucleosides. In various embodiments, an oligonucleotide with one or more spacers includes multiple segments with at most 6 or at most 7 linked nucleosides. In various embodiments, every segment of an oligonucleotide with one or more spacers has at most 6 or at most 7 linked nucleosides. For example, the oligonucleotide may be a 20mer and include two spacers that divide the 20mer into three separate segments of 6 linked nucleosides each. Therefore, each segment of the oligonucleotide has at most 6 linked nucleosides. For example, the oligonucleotide may be a 23mer and include two spacers that divide the 23mer into three separate segments of 7 linked nucleosides each. Therefore, each segment of the oligonucleotide has at most 7 linked nucleosides. Generally, oligonucleotides comprising one or more spacers are described in reference to a corresponding parent oligonucleotide or a corresponding oligonucleotide variant. Example oligonucleotides comprising one or spacers include any of SEQ ID NOs: 301692-301742 or the sequences in Tables 4A-4C.

In various embodiments, one or more spacers may be located at one or more positions of an oligonucleotide. A spacer may be located between a first position and a second position of the oligonucleotide. As used herein, a spacer located between a first position and second position encompasses the spacer being located at the first position, located at the second position, or located at any position of the oligonucleotide sandwiched by the first position and the second position.

The term “gapmer antisense oligonucleotide,” “gapmer AON,” or “gapmer AON variant” refers to an AON with at least three distinct structural regions including a 5′-wing region, a central region, and a 3′-wing region, in ‘5→3’ orientation. The central region comprises a stretch of nucleosides that enable recruitment and activation of RNAseH. For example, the central region comprises linked DNA nucleosides, 2′-Fluoro Arabino Nucleic Acids (FANA), and Fluoro Cyclohexenyl nucleic acid (F-CeNA). Example gapmer AONs or gapmer oligonucleotide variants include any of SEQ ID NOs: 301595-301607. In various embodiments, gapmer AONs comprise a sequence that shares at least 85%, at least 90%, at least 95%, at least 98%, or 100% identity with an equal length portion of any one of SEQ ID NOs: 1-954, 1914-149354, 149362-158581, or 167805-301566. In various embodiments, a gapmer AON may include one or more spacers. Example gapmer AONs comprising one or spacers comprise a sequence that shares at least 85%, at least 90%, at least 95%, at least 98% identity, or 100% identity with an equal length portion of any one of SEQ ID NOs: SEQ ID NOs: 301692-301742 or the sequences in Tables 4A-4C.

The term “pharmaceutically acceptable salt(s)” as used herein refers to salts of acidic or basic groups that may be present in antisense oligonucleotides used in the present compositions. Antisense oligonucleotides included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, e.g., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (e.g., 1,1′-methylene-bis-(2-hydroxy-3-naphthoate)) salts. Antisense oligonucleotides included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of AONs that include a nucleotide sequence of any of SEQ ID NOs: 1-954, 1914-149354, 149362-158581, 167805-301566, SEQ ID NOs: 301692-301742 or the sequences in Tables 4A-4C.

Antisense oligonucleotides of the disclosure may contain one or more chiral centers, groups, linkages, and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S” (or “Rp” or “Sp”) depending on the configuration of substituents around the stereogenic atom, for example, a stereogenic carbon, phosphorus, or sulfur atom. In some embodiments, one or more linkages of the compound may have a Rp or Sp configuration (e.g., one or more phosphorothioate linkages have either a Rp or Sp configuration). The configuration of each phosphorothioate linkage may be independent of another phosphorothioate linkage (e.g., one phosphorothioate linkage has a Rp configuration and a second phosphorothioate linkage has a Sp configuration). The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. Individual stereoisomers of antisense oligonucleotides of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase super critical fluid chromatography, chiral-phase simulated moving bed chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Individual stereoisomers antisense oligonucleotides of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase super critical fluid chromatography, chiral-phase simulated moving bed chromatography, chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The antisense oligonucleotides disclosed herein can exist in solvated as well as unsolvated forms with pharmaceutically acceptable solvents such as water, ethanol, and the like, and it is intended that the invention embrace both solvated and unsolvated forms.

The invention also embraces isotopically labeled compounds of the invention (e.g., isotopically labeled antisense oligonucleotides) which are identical to those recited herein, except that one or more atoms are replaced by an atom having an atomic mass or mass number different from the atomic mass or mass number most abundantly found in nature. Examples of isotopes that can be incorporated into compounds of the invention include isotopes of hydrogen, carbon, nitrogen, oxygen, phosphorus, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F, and ³⁶Cl, respectively.

Certain isotopically labeled disclosed compounds (e.g., those labeled with ³H and ¹⁴C) are useful in compound and/or substrate tissue distribution assays. Tritiated (i.e., ³H), carbon-14 (i.e., ¹⁴C), or ³⁵S phosphorothioate isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

As used herein, “2′-O-(2-methoxyethyl)” (also 2′-MOE and 2′-O(CH₂)₂OCH₃ and MOE) refers to an O-methoxyethyl modification of the 2′ position of a furanose ring. A 2′-O-(2-methoxyethyl) is used interchangeably as “2′-O-methoxyethyl” in the present disclosure. A sugar moiety in a nucleoside modified with 2′-MOE is a modified sugar.

As used herein, “2′-MOE nucleoside” (also 2′-O-(2-methoxyethyl) nucleoside) means a nucleoside comprising a 2′-MOE modified sugar moiety.

As used herein, “2′-substituted nucleoside” means a nucleoside comprising a substituent at the 2′-position of the furanose ring other than H or OH. In certain embodiments, 2′ substituted nucleosides include nucleosides with bicyclic sugar modifications.

As used herein, “5-methyl cytosine” (5-MeC) means a cytosine modified with a methyl group attached to the 5 position. A 5-methyl cytosine (5-MeC) is a modified nucleobase.

As used herein, “bicyclic sugar” means a furanose ring modified by the bridging of two atoms. A bicyclic sugar is a modified sugar.

As used herein, “bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

As used herein, “cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

As used herein, “constrained ethyl nucleoside” (also cEt nucleoside) means a nucleoside comprising a bicyclic sugar moiety comprising a 4′-CH(CH₃)—O-2′ bridge. In some embodiments, cEt can be modified. In some embodiments, the cEt can be S-cEt (in an S-constrained ethyl 2′-4′-bridged nucleic acid). In some other embodiments, the cEt can be R-cEt.

As used herein, “internucleoside linkage” refers to the atom or group that links the 3′ and 5′ position of the sugar or corresponding positions of a sugar mimetic. In some embodiments, as used herein, “non-natural linkage” refers to a “modified internucleoside linkage.”

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence. As an example to the contrary, two nucleosides separated by a spacer are not contiguous.

As used herein, “modified nucleobase” means any nucleobase other than adenine, cytosine, guanine, thymine, or uracil. Examples of a modified nucleobase include 5-methyl cytosine, pseudouridine, or 5-methoxyuridine. An “unmodified nucleobase” means the purine bases adenine (A) and guanine (G), and the pyrimidine bases thymine (T), cytosine (C), and uracil (U).

As used herein, a “spacer” refers to a nucleoside-replacement group (e.g., a non-nucleoside group that replaces a nucleoside present in a parent oligonucleotide). The spacer is characterized by the lack of a nucleotide base and by the replacement of the nucleoside sugar moiety with a non-sugar substitute. The non-sugar substitute group of a spacer lacks an aldehyde, ketone, acetal, ketal, hemiacetal or hemiketal group. The non-sugar substitute group of a spacer is thus capable of connecting to the 3′ and 5′ positions of the nucleosides adjacent to the spacer through an internucleoside linker as described herein, but not capable of forming a covalent bond with a nucleotide base (i.e., not capable of linking a nucleobase to another group, such as an internucleoside linkage, conjugate group, or terminal group in an oligonucleotide). Generally, an antisense oligonucleotide with a spacer is described in relation to a parent antisense oligonucleotide, wherein the spacer replaces a nucleoside of the parent antisense oligonucleotide. In all embodiments of the present disclosure, a spacer cannot hybridize to a nucleoside comprising a nucleobase at the corresponding position of a transcript (e.g., pre-mRNA or mRNA transcript), within the numerical order of the length of the AON (i.e., if the spacer is positioned after nucleoside 4 of an AON (i.e., at position 5 from the 5′-end), the spacer is not complementary to the nucleoside (A, C, G, or U) at the same corresponding position of the target transcript)).

As used herein, a “modified nucleoside” means a nucleoside having, independently, a modified sugar moiety and/or modified nucleobase. A universal base is a modified nucleobase that can pair with any one of the five unmodified nucleobases. Modified nucleosides include abasic nucleosides, which lack a nucleobase. However, modified nucleosides do not include spacers or other groups that are incapable of linking a nucleobase.

As used herein, “linked nucleosides” are nucleosides that are connected in a contiguous sequence (i.e., no additional nucleosides are presented between those that are linked). In various embodiments, an oligonucleotide may have different segments of linked nucleosides connected through a spacer. Here, the spacer (i.e., nucleoside replacement) is not considered a nucleoside and therefore, divides up the oligonucleotide into two segments of linked nucleosides. The oligonucleotide may have a first segment of Y linked nucleosides (e.g., Y nucleosides that are connected in a contiguous sequence), followed by a spacer, and then a second segment of Z linked nucleosides. Here, the Y and Z linked nucleosides is described in either the 5′ to 3′ direction or the 3′ to 5′ direction. In various embodiments, the first segment consists of 7 or fewer linked nucleosides (e.g., Y=7 or fewer) whereas the second segment comprises 8 or more linked nucleosides (e.g., Z=8 or more).

As used herein, “locked nucleic acid” or “LNA” or “LNA nucleosides” means nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA; wherein R is H, C₁-C₁₂ alkyl, or a protecting group (see U.S. Pat. No. 7,427,672, issued on Sep. 23, 2008).

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. A-L-methyleneoxy (4′-CH₂—O-2′), an isomer of methyleneoxy (4′-CH₂—O-2′) LNA is also encompassed within the definition of LNA, as used herein.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoosteen or reversed Hoosteen hydrogen bonding between complementary nucleobases.

As used herein, “increasing the amount of activity” refers to more transcriptional expression, more accurate splicing resulting in full length mature mRNA and/or protein expression, and/or more activity relative to the transcriptional expression or activity in an untreated or control sample.

As used herein, “mismatch” or “non-complementary nucleobase” refers to the case when a group (e.g., nucleobase) of a first nucleic acid is not capable of pairing with the corresponding group (e.g., nucleobase) of a second or target nucleic acid.

As used herein, “modified internucleoside linkage” refers to a substitution or any change from a naturally occurring internucleoside linkage (e.g., a phosphodiester internucleoside bond). “Phosphorothioate linkage” is a modified internucleoside linkage in which one of the non-bridging oxygen atoms of a phosphodiester internucleoside linkage is replaced with a sulfur atom.

As used herein. “modified oligonucleotide” means an oligonucleotide comprising at least one (i.e., one or more) modified internucleoside linkage, modified sugar, and/or modified nucleobase.

As used herein, “modified sugar” or “modified sugar moiety” means a modified furanosyl sugar moiety or a modified sugar moiety having other than a furanosyl moiety that can link a nucleobase to another group, such as an internucleoside linkage, conjugate group, or terminal group in an oligonucleotide.

As used herein, “monomer” means a single unit of an oligomer. Monomers include, but are not limited to, nucleosides and nucleotides, whether naturally occurring or modified.

As used herein, “motif” means the pattern of unmodified and modified nucleosides in an antisense compound.

As used herein, “natural sugar moiety” means a sugar moiety found in DNA (2′-H) or RNA (2-OH).

As used herein, “naturally occurring internucleoside linkage” means a 3′ to 5′ phosphodiester linkage.

As used herein, “nucleobase” means a heterocyclic moiety capable of pairing with a base of another nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, “non-complementary nucleobases” refers to a pair of nucleobases that do not form hydrogen bonds with one another or otherwise support hybridization.

As used herein, “nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, non-coding RNA, small interfering ribonucleic acids (siRNA), short-hairpin RNA (shRNA), circular RNA, circular DNA, and microRNAs (miRNA).

As used herein. “nucleobase sequence” means the order of nucleobases independent of any sugar, linkage, and/or nucleobase modification.

As used herein, “nucleoside” refers to a nucleobase linked to a sugar. The term “nucleoside” also includes a “modified nucleoside” which has independently, a modified sugar moiety and/or modified nucleobase.

As used herein, “oligonucleotide unit” refers to either a nucleoside (e.g., a nucleoside which includes a sugar and/or a nucleobase) or a nucleoside-replacement group (e.g., a spacer) of the oligonucleotide. An oligonucleotide unit encompasses nucleosides, modified nucleosides, and spacers. For example, an oligonucleotide may have 20 oligonucleotide units, wherein one of the oligonucleotide units is a spacer and the other 19 oligonucleotide units are nucleosides or modified nucleosides. As another example, an oligonucleotide may have 20 oligonucleotide units, wherein two of the oligonucleotide units are spacers, and the other 18 oligonucleotide units are nucleosides or modified nucleosides.

As used herein. “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by a phosphorodiamidate or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

As used herein, “nucleotide” means a nucleoside having a phosphate group covalently linked to the sugar portion of the nucleoside.

As used herein, “oligomeric compound” or “oligomer” means a polymer of linked monomeric subunits which is capable of hybridizing to at least a region of a nucleic acid molecule.

As used herein. “oligonucleotide” means a polymer of one or more segments of linked nucleosides each of which can be modified or unmodified, independent one from another.

The disclosure provides methods for treating, ameliorating, or preventing a disease such as, but not limited to, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA). Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism, in a patient, comprising administering to a patient a composition comprising a therapeutically effective amount of an antisense oligonucleotide, and a pharmaceutically acceptable excipient.

Also provided herein are methods for treating, ameliorating, or preventing a disease, the method comprising administering to a patient a composition comprising a therapeutically effective amount of an antisense oligonucleotide and a pharmaceutically acceptable excipient. For example, in some embodiments, methods for treating, ameliorating, or preventing a disease comprise administering a pharmaceutically acceptable composition, for example, a pharmaceutically acceptable formulation, that includes one or more antisense oligonucleotides, to a patient. Antisense oligonucleotides disclosed herein can target transcripts (e.g., mRNA or pre-mRNA transcripts) of any one of PPM1A, ATXN2, SOD1, or MAPT. Antisense oligonucleotides can modulate expression of transcripts for treating a disease disclosed herein.

The present disclosure also provides pharmaceutical compositions comprising antisense oligonucleotides as disclosed herein formulated together with one or more pharmaceutically or cosmetically acceptable excipients. These formulations include those suitable for oral, sublingual, intratracheal, intranasal, vaginal, rectal, topical, transdermal, pulmonary, intrathecal, intracisternal, intrathecal, intrathalamic, intracerebroventricular, intraocular, buccal, and parenteral (e.g., subcutaneous, intramuscular, intradermal, intraduodenal, or intravenous) administration, or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular antisense oligonucleotide being used.

The present invention also provides a pharmaceutical composition comprising an antisense oligonucleotide, or a pharmaceutically acceptable salt thereof. The present disclosure also provides methods that include the use of pharmaceutical compositions comprising antisense oligonucleotides as disclosed herein (e.g., an AON comprising any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C) formulated together with one or more pharmaceutically acceptable excipients. Exemplary compositions provided herein include compositions comprising an antisense oligonucleotide, as described above, and one or more pharmaceutically acceptable excipients. Formulations include those suitable for oral, sublingual, intratracheal, intranasal, rectal, vaginal, topical, transdermal, pulmonary, intrathecal, intracisternal, intrathecal, intrathalamic, intracerebroventricular, intraocular, buccal, and parenteral (e.g., subcutaneous, intramuscular, intradermal, intraduodenal, or intravenous) administration, or for topical use. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

### Antisense Therapeutics

Antisense therapeutics are a class of nucleic acid-based compounds that can be used to inhibit gene expression. Antisense therapeutics may be single- or double-stranded deoxyribonucleic acid (DNA)-based, ribonucleic acid (RNA)-based, or DNA/RNA chemical analogue compounds. In general, antisense therapeutics are designed to include a nucleotide sequence that is complementary or nearly complementary to an mRNA or pre-mRNA sequence of a given gene in order to promote binding between the antisense therapeutic and the pre-mRNA or mRNA. Examples of given genes disclosed herein include, but are not limited to PPM1A, ATXN2, SOD1, or MAPT.

Without being bound by theory, it is believed that in most instances antisense therapeutics act by binding to an mRNA or pre-mRNA, thereby inhibiting protein translation, altering pre-mRNA splicing into mature mRNA, and/or causing destruction of mRNA. In most instances, the antisense therapeutic nucleotide sequence is complementary to a portion of a targeted gene's, mRNA's, or pre-mRNA's sense sequence. Antisense therapeutics described herein are oligonucleotide-based compounds that include an oligonucleotide sequence complementary to a gene sense, pre-mRNA sense, and/or mRNA sense sequence, or a portion thereof. Antisense therapeutics described herein can also be nucleotide chemical analog-based compounds capable of binding to a gene sense, pre-mRNA sense, and/or mRNA sense sequence, or a portion thereof. Antisense therapeutics include antisense oligonucleotides, shRNAs, siRNAs, PNAs, LNAs, and morpholino oligomers.

Antisense oligonucleotides (AONs) are short oligonucleotide-based sequences that include an oligonucleotide sequence complementary to a target RNA sequence. AONs are typically between 8 to 50 nucleotides in length, for example, 18 nucleotides in length. 20 nucleotides in length, or 23 nucleotides in length. AONs may include chemically modified nucleosides (for example, 2′-O-methylated nucleosides or 2′-O-(2-methoxyethyl) nucleosides) as well as modified internucleoside linkages (for example, phosphorothioate linkages). AONs described herein include oligonucleotide sequences that are complementary to RNA sequences, such as mRNA or pre-mRNA transcripts. AONs described herein can include chemically modified nucleosides and modified internucleoside linkages (for example, phosphorothioate linkages).

Peptide nucleic acids (PNAs) are short, artificially synthesized polymers with a structure that mimics DNA or RNA. PNAs include a backbone composed of repeating N-(2-aminoethyl)-glycine units linked by peptide bonds. PNAs described herein can be used as antisense therapeutics that bind to mRNA or pre-mRNA sequences with high specificity and inhibit target gene expression.

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. LNAs described herein can be used as antisense therapeutics that bind to RNA sequences with high specificity and inhibit target gene expression.

Morpholino oligomers are oligonucleotide compounds that include DNA bases attached to a backbone of methylenemorpholine rings linked through phosphorodiamidate groups. Morpholino oligomers of the present invention can be designed to bind to specific RNA sequences of interest (for example, mRNA or pre-mRNA sequences of interest), thereby preventing gene expression. Morpholino oligomers described herein can be used as antisense therapeutics that bind to mRNA sequences with high specificity and inhibit gene expression. Morpholino oligomers described herein can also be used to bind pre-mRNA sequences, altering pre-mRNA splicing and gene expression.

Small hairpin RNAs (shRNAs) are generally RNA molecules with a hairpin-like structure that can be used to silence gene expression. shRNAs are generally expressed from plasmids encoding the shRNA sequence, and can be expressed from viral vectors to allow lentiviral, adenoviral, or adeno-associated viral expression. Without being bound by theory, it is believed that shRNA inhibits gene expression by taking advantage of RNA interference (RNAi) processes. In brief, the shRNA transcript is processed by Drosha and Dicer, and then loaded onto the RNA-induced silencing complex (RISC), allowing targeting of specific mRNA, and either mRNA degradation or repression of protein translation. shRNAs described herein can inhibit target gene expression.

Small interfering RNAs (siRNAs) are double-stranded RNA molecules of approximately 20-25 base pairs in length that take advantage of RNAi machinery (e.g., Drosha and RISC) to bind and target mRNA for degradation. siRNAs are not dependent upon plasmids or vectors for expression, and can generally be delivered directly to a target cell, for instance, by transfection. siRNAs are double-stranded RNA sequences that include an RNA sequence complementary to a mRNA sequence, and which prevent protein translation.

The number of nucleotides included in an antisense therapeutic, for example, an antisense oligonucleotide described herein may vary. For example, in some embodiments, the antisense oligonucleotide is from 12 to 15 oligonucleotide units in length. In some embodiments, the antisense oligonucleotide is from 15 to 20 oligonucleotide units in length. In some embodiments, the antisense oligonucleotide is from 20 to 40 oligonucleotide units in length. In some embodiments, the antisense oligonucleotide is from 20 to 22 oligonucleotide units in length. In some embodiments, the antisense oligonucleotide is from 22 to 40 oligonucleotide units in length. In some embodiments, the antisense oligonucleotide is from 20 to 30, 25 to 35, or 30 to 40 oligonucleotide units in length. In particular embodiments, the antisense oligonucleotide is 18 oligonucleotide units in length. In particular embodiments, the antisense oligonucleotide is 20 oligonucleotide units in length. In particular embodiments, the antisense oligonucleotide is 23 oligonucleotide units in length.

### Antisense Oligonucleotides

Antisense oligonucleotides (AONs) described herein are short synthetic oligonucleotide sequence complementary to a portion of a gene product, such as any of a PPM1A, ATXN2, SOD1, or MAPT transcript (for example, a PPM1A, ATXN2, SOD1, or MAPT mRNA transcript, or a PPM1A, ATXN2, SOD1, or MAPT pre-mRNA transcript).

In various embodiments, AONs include linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a target gene product, such as a mRNA or pre-mRNA sequence. In some embodiments, an AON can include a non-duplexed oligonucleotide. In some embodiments, an AON can include a duplex of two oligonucleotides where the first oligonucleotide includes a nucleotide sequence that is completely or almost completely complementary to a target mRNA sequence and the second oligonucleotide includes a nucleotide sequence that is complementary to the nucleotide sequence of the first oligonucleotide. AON binding specificity can be assessed via measurement of parameters such as dissociation constant, melting temperature (Tm), or other criteria such as changes in protein or RNA expression levels or other assays that measure target activity or expression.

An AON, such as disclosed herein, may be an oligonucleotide sequence of 5 to 100 oligonucleotide units in length, for example, 10 to 40 oligonucleotide units in length, for example, 14 to 40 oligonucleotide units in length, 10 to 30 oligonucleotide units in length, for example, 14 to 30 oligonucleotide units in length, for example, 14 to 25 oligonucleotide units in length, 15 to 22 oligonucleotide units in length, 18 to 21 oligonucleotide units in length, or 18, 19, 20, 21, 22, 23, 24, or 25 oligonucleotide units in length. In particular embodiments, an AON is 18 oligonucleotide units in length. In particular embodiments, an AON is 20 oligonucleotide units in length. In particular embodiments, an AON is 23 oligonucleotide units in length. In particular embodiments, an AON includes 20 linked nucleosides. As used herein, “parent oligonucleotides” refer to AONs including 20 linked nucleosides.

AONs described herein also include antisense oligonucleotides comprising the oligonucleotide sequences listed in Tables 1A-1D. Specifically, Table 1A shows example PPM1A AONs. In various embodiments, PPM1A AONs comprise a sequence of any one of SEQ ID NOs: 1-954. Table 1B shows example ATXN2 AONs. In various embodiments, ATXN2 AONs comprise a sequence of any one of SEQ ID NOs: 1914-149354. Table 1C shows example SOD1 AONs. In various embodiments, SOD1 AONs comprise a sequence of any one of SEQ ID NOs: 149362-158581. Table 1D shows example MAPT AONs. In various embodiments, MAPT AONs comprise a sequence of any one of SEQ ID NOs: 167805-301566.

In various embodiments, the AONs shown in Tables 1A-1D comprise at least one nucleoside linkage selected from a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In particular embodiments, disclosed herein are gapmer oligonucleotides comprising a sequence that shares at least 85% identity with an equal length portion of any one of SEQ ID NOs: 1-954, 1914-149354, 149362-158581, 167805-301566, SEQ ID NOs: 301692-301742 or the sequences in Tables 4A-4C. In particular embodiments, disclosed herein are gapmer oligonucleotides comprising a sequence that shares at least 90% identity with an equal length portion of any one of SEQ ID NOs: 1-954, 1914-149354, 149362-158581, 167805-301566, SEQ ID NOs: 301692-301742 or the sequences in Tables 4A-4C. In particular embodiments, disclosed herein are gapmer oligonucleotides comprising a sequence that shares at least 95% identity with an equal length portion of any one of SEQ ID NOs: 1-954, 1914-149354, 149362-158581, 167805-301566, SEQ ID NOs: 301692-301742 or the sequences in Tables 4A-4C. In particular embodiments, disclosed herein are gapmer oligonucleotides comprising a sequence that shares at least 98% identity with an equal length portion of any one of SEQ ID NOs: 1-954, 1914-149354, 149362-158581, 167805-301566, SEQ ID NOs: 301692-301742 or the sequences in Tables 4A-4C. In particular embodiments, disclosed herein are gapmer oligonucleotides comprising a sequence that shares 100% identity with an equal length portion of any one of SEQ ID NOs: 1-954, 1914-149354, 149362-158581, 167805-301566, SEQ ID NOs: 301692-301742 or the sequences in Tables 4A-4C.

### Oligonucleotide Variants

In various embodiments, AONs include different variants (e.g., AONs of different lengths), hereafter referred to as AON variants. An AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 18 to 21 nucleobases in length, for example, or 18, 19, 20, 21, 22, 23, 24, 25, 26, 27, 28, 29, or 30 nucleobases in length. An AON variant may be an oligonucleotide sequence complementary to a portion of a target mRNA sequence or a target pre-mRNA sequence.

In various embodiments, an AON variant represents a modified version of a corresponding parent oligonucleotide that includes a nucleobase sequence selected from any one of SEQ ID NOs: 1-954, 1914-149354, 149362-158581, or 167805-301566. In some embodiments, an AON variant includes a nucleobase sequence that represents a shortened version of a nucleobase sequence of an AON selected from any one of SEQ ID NOs: 1-954, 1914-149354, 149362-158581, or 167805-301566. As one example, if a parent oligonucleotide includes a 20mer (e.g., 20 nucleotide bases in length), a variant (e.g., an AON variant) may include a shorter version (e.g., 15mer, 16mer, 17mer, 18mer, or 19mer) of the 20mer parent oligonucleotide or a longer version (e.g., 21mer, 22mer, 23mer, 24mer, 25mer, 26mer, 27mer, 28mer, 29mer, or 30mer) of the 20mer parent oligonucleotide. In one embodiment, a nucleobase sequence of an AON variant differs from a corresponding nucleobase sequence of a parent oligonucleotide in that 1, 2, 3, 4, 5, or 6 nucleotide bases are removed from or added to one or both of the 3′ and 5′ ends of the nucleobase sequence of the parent oligonucleotide.

In one embodiment, the corresponding AON variant may include a 18mer where one nucleotide base was removed from each of the 3′ and 5′ end of a 20mer included in the parent oligonucleotide. For example, a 18mer AON variant may comprise a sequence in which one nucleotide base is removed from each of the 3′ and 5′ end of any one of SEQ ID NOs: 1-954, 1914-149354, 149362-158581, or 167805-301566.

In one embodiment, the corresponding AON variant may include a 18mer where two nucleotide bases were removed from the 3′ end of a 20mer included in the parent oligonucleotide. For example, a 18mer AON variant may comprise a sequence in which two nucleotide bases are removed from the 3′ end of any one of SEQ ID NOs: 1-954, 1914-149354, 149362-158581, or 167805-301566. In one embodiment, the corresponding AON variant may include a 18mer where two nucleotide bases were removed from the 5′ end of a 20mer included in the parent oligonucleotide. For example, a 18mer AON variant may comprise a sequence in which two nucleotide bases are removed from the 5′ end of any one of SEQ ID NOs: 1-954, 1914-149354, 149362-158581, or 167805-301566.

In one embodiment, the corresponding AON variant may include a 19mer where one nucleotide base was removed from either the 3′ or 5′ end of a 20mer included in the parent oligonucleotide. For example, a 19mer AON variant may comprise a sequence in which one nucleotide base is removed from the 3′ end of any one of SEQ ID NOs: 1-954, 1914-149354, 149362-158581, or 167805-301566. For example, a 19mer AON variant may comprise a sequence in which one nucleotide base is removed from the 5′ end of any one of SEQ ID NOs: 1-954, 1914-149354, 149362-158581, or 167805-301566.

In one embodiment, the corresponding AON variant may include a 23mer where three nucleotide bases are added to either the 3′ or 5′ end of the 20mer included in the parent oligonucleotide. For example, a 19mer AON variant may comprise a sequence in which three nucleotide bases are added to either the 3′ end or the 5′ end of any one of SEQ ID NOs: 1-954, 1914-149354, 149362-158581, or 167805-301566.

Example sequences of AON variants are shown below in Tables 2A-2B.

TABLE 2A
Example MAPT AON Variant Sequences.

| SEQ ID NO: | Length | MAPT AON Sequence (5′-3′)* |
|---|---|---|
| 301590 | 18 oligonucleotide units | CCGTTTTCTTACCACCCT |

TABLE 2B
Example PPM1A AON Variant Sequences

| SEQ ID NO: | Length | PPM1A AON Sequence (5′-3′)* |
|---|---|---|
| 301591 | 23 oligonucleotide units | TCCACTTGGCAAACCGATCACAG |
| 301592 | 23 oligonucleotide units | GTCCACTTGGCAAACCGATCACA |
| 301593 | 23 oligonucleotide units | CCACTTGGCAAACCGATCACAGC |
| 301594 | 23 oligonucleotide units | AGTCCACTTGGCAAACCGATCAC |

In Tables 2A and 2B, “*” indicates that at least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

### Gapmer AONs

In particular embodiments, AONs disclosed herein have a gapmer design or structure also referred to herein as “gapmer” or “gapmer AONs.” In a gapmer structure the AON comprises at least three distinct structural regions including a 5′-wing region, a central region, and a 3′-wing region, in 5→3′ orientation.

In various embodiments, the 5′ wing region includes one, two, three, four, five, six, seven, eight, nine, or ten oligonucleotide units. In various embodiments, at least one of the oligonucleotide units of the 5′ wing region includes a spacer. In various embodiments, the 5′ wing region includes one, two, three, four, five, six, seven, eight, nine, or ten linked nucleosides. In various embodiments, the 3′ wing region includes one, two, three, four, five, six, seven, eight, nine, or ten oligonucleotide units. In various embodiments, at least one of the oligonucleotide units of the 3′ wing region includes a spacer. In various embodiments, the 3′ wing region includes one, two, three, four, five, six, seven, eight, nine, or ten linked nucleosides. The 5′ and 3′ wing regions (also termed flanking regions) comprise at least one oligonucleotide unit that is adjacent to the central region, which, in some embodiments, comprises a stretch of contiguous nucleosides. The 5′ and 3′ wing regions may be symmetrical or asymmetrical with respect to the number of oligonucleotide units or linked nucleosides they include.

In various embodiments, the 5′ wing region comprises one or more RNA nucleosides (e.g, ribonucleosides). In various embodiments, the 5′ wing region comprises one or more DNA nucleosides (e.g, deoxyribonucleosides). In various embodiments, the 5′ wing region comprises both RNA nucleosides and DNA nucleosides. In various embodiments, the 3′ wing region comprises one or more RNA nucleosides. In various embodiments, the 3′ wing region comprises one or more DNA nucleosides. In various embodiments, the 3′ wing region comprises both RNA nucleosides and DNA nucleosides.

In various embodiments, the central region includes one, two, three, four, five, six, seven, eight, nine, ten, eleven, twelve, thirteen, fourteen, fifteen, sixteen, seventeen, eighteen, nineteen, or twenty oligonucleotide units. In various embodiments, the central region includes one, two, three, four, five, six, seven, eight, nine, ten, eleven, twelve, thirteen, fourteen, fifteen, sixteen, seventeen, eighteen, nineteen, or twenty linked nucleosides. In some embodiments, the central region comprises a stretch of nucleosides that enable recruitment and activation of RNAseH. In some embodiments, the central region comprises one or more of linked DNA nucleosides, 2′-Fluoro Arabino Nucleic Acids (FANA), and Fluoro Cyclohexenyl nucleic acid (F-CeNA). In some embodiments, all nucleosides of the central region are DNA nucleosides. In some embodiments, the central region comprises a contiguous stretch of 5-16 DNA nucleosides. In some embodiments, the central region comprises a contiguous stretch of 6-15, 7-14, 8-13, or 9-11 DNA nucleosides. In various embodiments, the central region comprises a mix of DNA nucleosides and RNA nucleosides. In various embodiments, at least one oligonucleotide unit of the central region is a spacer. In some embodiments, one oligonucleotide unit of the central region is a spacer. In some embodiments, two oligonucleotide units of the central region are spacers.

In some embodiments, all of the nucleosides of the central region are DNA nucleosides. In further embodiments the central region may consist of a mixture of DNA nucleosides and other nucleosides (2′-Fluoro Arabino Nucleic Acids (FANA), and Fluoro Cyclohexenyl nucleic acid (F-CeNA)) capable of mediating RNase H cleavage. In some embodiments, at least 50% of the nucleosides of the central region are DNA nucleosides, such as at least 60%, at least 65%, at least 70%, at least 75%, at least 80%, at least 85%, at least 90%, at least 95%, or 100% DNA nucleosides.

In particular embodiments, the AON includes a 5′ wing region of 5 linked nucleosides, a central region of 10 linked nucleosides, and a 3′ wing region of 5 linked nucleosides, also referred to as a 5-10-5 gapmer. In particular embodiments, the AON includes a 5′ wing region of 3 linked nucleosides, a central region of 8 linked nucleosides, and a 3′ wing region of 3 linked nucleosides, also referred to as a 3-8-3 gapmer. In particular embodiments, the AON includes a 5′ wing region of 3 linked nucleosides, a central region of 10 linked nucleosides, and a 3′ wing region of 3 linked nucleosides, also referred to as a 3-10-3 gapmer. In particular embodiments, the AON includes a 5′ wing region of 4 linked nucleosides, a central region of 10 linked nucleosides, and a 3′ wing region of 4 linked nucleosides, also referred to as a 4-10-4 gapmer. In particular embodiments, the AON includes a 5′ wing region of 4 linked nucleosides, a central region of 8 linked nucleosides, and a 3′ wing region of 4 linked nucleosides, also referred to as a 4-8-4 gapmer. In various embodiments, at least one of the nucleosides in any of the 3′ wing region, 5′ wing region, or central is replaced with a spacer. Embodiments of gapmer AONs with one or more spacers is described in further detail herein.

In particular embodiments, gapmer AONs disclosed herein comprise a sequence of any one of SEQ ID NOs: 1-954, 1914-149354, 149362-158581, or 167805-301566, wherein the gapmer AON includes a 5′ wing region comprising one or more RNA nucleosides, a central region comprising one or more DNA nucleosides, and a 3′ wing region comprising one or more RNA nucleosides. In particular embodiments, gapmer AONs disclosed herein comprise a sequence of any one of SEQ ID NOs: 1-954, 1914-149354, 149362-158581, or 167805-301566 are 5-10-5 gapmers (e.g., 5′ wing region of 5 linked nucleosides, a central region of 10 linked nucleosides, and a 3′ wing region of 5 linked nucleosides). In particular embodiments, gapmer AONs disclosed herein comprise a sequence of any one of SEQ ID NOs: 1-954, 1914-149354, 149362-158581, or 167805-301566 are 6-11-6 gapmers (e.g., 5′ wing region of 6 linked nucleosides, a central region of 11 linked nucleosides, and a 3′ wing region of 6 linked nucleosides). In particular embodiments, gapmer AONs disclosed herein comprise a sequence of any one of SEQ ID NOs: 1-954, 1914-149354, 149362-158581, or 167805-301566 are 5-8-5 gapmers (e.g., 5′ wing region of 5 linked nucleosides, a central region of 8 linked nucleosides, and a 3′ wing region of 5 linked nucleosides).

Further example Gapmer AONs described herein can include those identified below in Tables 3A-3D. Specifically, Table 3A shows example PPM1A gapmer AONs, Table 3B shows example ATXN2 gapmer AONs, Table 3C shows example SOD1 gapmer AONs, and Table 3D shows example MAPT gapmer AONs. In various embodiments, all cytosines of gapmer AONs shown in Tables 3A-3D are 5 methylcytosines (5-MeC).

TABLE 3A
Example PPM1A Gapmer AONs and Gapmer variant AONs. In the gapmer
structure, “e” refers to a ribonucleoside, such as a 2′-MOE
modified ribonucleoside, and “d10” or “d11” denotes 10 or 11 DNA
deoxyribonucleosides, respectively

| SEQ ID NO: | PPM1A AON Sequence (5′-3′) | Gapmer structure |
|---|---|---|
| 301595 | TCCACTTGGCAAACCGATCACAG | eeeeee-d11-eeeeee |
| 301596 | GTCCACTTGGCAAACCGATCACA | eeeeee-d11-eeeeee |
| 301597 | CCACTTGGCAAACCGATCACAGC | eeeeee-d11-eeeeee |
| 301598 | AGTCCACTTGGCAAACCGATCAC | eeeeee-d11-eeeeee |
| 301599 | CCACTTGGCAAACCGATCAC | eeeee-d10-eeeee |
| 301600 | GCCCATCATACACAGCAAAG | eeeee-d10-eeeee |

TABLE 3B
Example ATXN2 Gapmer AONs. In the gapmer structure, “e” refers to
a ribonucleoside, such as a 2′-MOE modified ribonucleoside, and
“d10” denotes 10 deoxyribonucleosides.

| SEQ ID NO: | ATXN2 AON Sequence (5′-3′) | Gapmer structure |
|---|---|---|
| 301601 | GCTAACTGGTTTGCCCTTGC | eeeee-d10-eeeee |
| 301602 | GTGGGATACAAATTCTAGGC | eeeee-d10-eeeee |
| 301603 | GGAGCTGGAGAACCATGAGC | eeeee-d10-eeeee |
| 301604 | TGTACTTCACATTTGGAGCC | eeeee-d10-eeeee |

TABLE 3C
Example SOD1 Gapmer AON. In the gapmer structure, “e” refers to a
ribonucleoside, such as a 2′-MOE modified ribonucleoside, and
“d10” denotes 10 deoxyribonucleosides.

| SEQ ID NO: | SOD1 AON Sequence (5′-3′) | Gapmer structure |
|---|---|---|
| 301605 | CAGGATACATTTCTACAGCT | eeeee-d10-eeeee |

TABLE 3D
Example MAPT Gapmer AONs and Gapmer variant AONs. In the gapmer
structure, “e” refers to a ribonucleoside, such as a 2′-MOE modified
ribonucleoside, and “d8” or “d10” denotes 8 or 10
deoxyribonucleosides, respectively.

| SEQ ID NO: | MAPT AON Sequence (5′-3′) | Gapmer structure |
|---|---|---|
| 301606 | CCGTTTTCTTACCACCCT | eeeee-d8-eeeee |
| 301607 | CCGTTTTCTTACCACCCTAA | eeeee-d10-eeeee |

In various embodiments, gapmer AONs, such as gapmer AONs comprising a sequence of any one of SEQ ID NOs: 1-954, 1914-149354, 149362-158581, or 167805-301566 or any of the gapmer AON sequences shown in Tables 3A-3D (e.g., SEQ ID NOs: 301595-301607), include a mixture of ribonucleosides and deoxyribonucleosides (including modified ribonucleosides and/or modified deoxyribonucleosides) in the 5′ wing region and/or the 3′ wing region.

In particular embodiments, gapmer AONs, such as gapmer AONs comprising a sequence of any one of SEQ ID NOs: 1-954, 1914-149354, 149362-158581, or 167805-301566 or any of the gapmer AON sequences shown in Tables 3A-3D (e.g., SEQ ID NOs: 301595-301607), include modified ribonucleosides in the 5′ wing region and/or the 3′ wing region. In particular embodiments, each nucleoside in the 5′ wing region and the 3′ wing region is a modified nucleoside e.g., a 2′-O-(2-methoxyethyl) (2′-MOE) nucleoside. For example, each guanonsine in the 5′ wing region or 3′ wing region may be a modified 2′-O-(2-methoxyethyl)guanosine. For example, each adenosine in the 5′ wing region or 3′ wing region may be a modified 2′-O-(2-methoxyethyl)adenosine. For example, each cytosine in the 5′ wing region and 3′ wing region may be a 2′-O-(2-methoxyethyl)-5-methylcytosine. For example, each thymidine in the 5′ wing region and the 3′ wing region may be a 2′-O-(2-methoxyethyl)thymidine.

In various embodiments, examplary gapmer AONs have one or more modified internucleoside linkages, such as any of a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Antisense Oligonucleotides with One or More Spacers

Embodiments disclosed herein include antisense oligonucleotides (AONs) comprising one or more spacers. In particular embodiments, an AON includes one spacer. In particular embodiments, an AON includes two spacers. In particular embodiments, an AON includes three spacers. Generally, a spacer refers to a nucleoside-replacement group lacking a nucleobase and wherein the nucleoside sugar moiety is replaced by a non-sugar substitute group. The non-sugar substitute group is not capable of linking to a nucleobase, but is capable of linking with the 3′ and 5′ positions of nucleosides adjacent to the spacer through an internucleoside linking group.

In various embodiments, AONs with one or more spacers include parent AONs (e.g., antisense oligonucleotides that are complementary to a portion of a target gene product, such as any one of a PPM1A, ATXN2, SOD1, or MAPT mRNA transcript) in which one or more of the nucleosides of the parent oligonucleotides (e.g., any of SEQ ID NOs: 1-954, 1914-149354, 149362-158581, or 167805-301566) are replaced with a spacer.

In various embodiments, AONs with one or more spacers include oligonucleotide variants (e.g., an antisense oligonucleotide that represents a modified version (e.g., shorter or longer) of a corresponding parent oligonucleotide) in which one or more of the nucleosides of the oligonucleotide variants (e.g., a shorter or longer version of a corresponding parent oligonucleotide that includes a nucleobase sequence selected from any one of SEQ ID NOs: 1-954, 1914-149354, 149362-158581, or 167805-301566) are replaced with a spacer.

In various embodiments, AONs with one or more spacers include gapmer AONs which include at least three distinct structural regions (e.g., a 5′-wing region, a central region, and a 3′-wing region, in ‘5→3’ orientation), in which one or more of the nucleosides of the gapmer AONs (e.g., gapmer AONs comprising a sequence of any one of SEQ ID NOs: 1-954, 1914-149354, 149362-158581, or 167805-301566 or any of the gapmer AON sequences shown in Tables 3A-3D (e.g., SEQ ID NOs: 301595-301607)) are replaced with a spacer. In particular embodiments, for a gapmer AON with one or more spacers, a nucleoside in the 5′-wing region is replaced with a spacer. In particular embodiments, for a gapmer AON with one or more spacers, a nucleoside in the 3′-wing region is replaced with a spacer. In particular embodiments, for a gapmer AON with one or more spacers, a nucleoside in the central region is replaced with a spacer. In particular embodiments, for a gapmer AON with one or more spacers, a nucleoside in the 5′-wing region is replaced with a spacer and a nucleoside in the central region is replaced with a spacer. In particular embodiments, for a gapmer AON with one or more spacers, a nucleoside in the 5′-wing region is replaced with a spacer and a nucleoside in the 3′-wing region is replaced with a spacer. In particular embodiments, for a gapmer AON with one or more spacers, a nucleoside in the central region is replaced with a spacer and a nucleoside in the 3′-wing region is replaced with a spacer.

In particular embodiments, the gapmer oligonucleotide comprises sugar modifications in any of the following patterns: eeeee-d10-eeeee, eeeee-d8-eeeee, eeeeee-d11-eeeeee, eee-d8-eee, eee-d10-eee, eeee-d10-eeee, and eeee-d8-eeee, wherein e=2′-MOE nucleoside and d=a deoxyribonucleoside. In such embodiments, a spacer may replace at least one “e” (e.g., a 2′-MOE nucleoside) or may replace at least one “d” (e.g., a deoxyribonucleoside) in the gapmer oligonucleotide.

As used herein, an “AON with one or more spacers” refers to any of a parent AON (e.g., any of SEQ ID NOs: 1-954, 1914-149354, 149362-158581, or 167805-301566) with one or more spacers, an AON variant (e.g., a shorter or longer version of a corresponding parent oligonucleotide that comprises a nucleobase sequence selected from any one of SEQ ID NOs: 1-954, 1914-149354, 149362-158581, or 167805-301566) with one or more spacers, or a gapmer AON (e.g., a gapmer AON comprising a sequence of any one of SEQ ID NOs: 1-954, 1914-149354, 149362-158581, or 167805-301566 or any of the gapmer AON sequences shown in Tables 3A-3D (e.g., SEQ ID NOs: 301595-301607)) with one or more spacers. In certain embodiments, an AON with one or more spacers may be an oligonucleotide with 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, or 25 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, AONs with one or more spacers are 25 oligonucleotide units in length. In particular embodiments, AONs with one or more spacers are 23 oligonucleotide units in length. In particular embodiments, AONs with one or more spacers are 21 oligonucleotide units in length. In particular embodiments. AONs with one or more spacers are 20 oligonucleotide units in length. In particular embodiments, AONs with one or more spacers are 19 oligonucleotide units in length. In particular embodiments, AONs with one or more spacers are 18 oligonucleotide units in length. In various embodiments, AONs with one or more spacers are at least 15, at least 16, at least 17, at least 18, at least 19, at least 20, at least 21, at least 22, at least 23, at least 24, or at least 25 oligonucleotide units in length. In various embodiments, the AONs with one or more spacers are at least 15 oligonucleotide units in length. In various embodiments, the AONs with one or more spacers are at least 16 oligonucleotide units in length. In various embodiments, the AONs with one or more spacers are at least 17 oligonucleotide units in length. In various embodiments, the AONs with one or more spacers are at least 18 oligonucleotide units in length. In various embodiments, the AONs with one or more spacers are at least 19 oligonucleotide units in length. In various embodiments, the AONs with one or more spacers are at least 20 oligonucleotide units in length. In various embodiments, the AONs with one or more spacers are at least 21 oligonucleotide units in length. In various embodiments, the AONs with one or more spacers are at least 22 oligonucleotide units in length. In various embodiments, the AONs with one or more spacers are at least 23 oligonucleotide units in length. In various embodiments, the AONs with one or more spacers are at least 24 oligonucleotide units in length. In various embodiments, the AONs with one or more spacers are at least 25 oligonucleotide units in length.

In various embodiments, an AON with one or more spacers comprises a sequence that shares at least 80% identity with an equal length portion of any one of SEQ ID NOs: 1-954, 1914-149354, 149362-158581, or 167805-301566. In various embodiments, an AON with one or more spacers comprises a sequence that shares at least 85% identity with an equal length portion of any one of SEQ ID NOs: 1-954, 1914-149354, 149362-158581, or 167805-301566. In various embodiments, an AON with one or more spacers comprises a sequence that shares at least 90% identity with an equal length portion of any one of SEQ ID NOs: 1-954, 1914-149354, 149362-158581, or 167805-301566. In various embodiments, an AON with one or more spacers comprises a sequence that shares at least 95% identity with an equal length portion of any one of SEQ ID NOs: 1-954, 1914-149354, 149362-158581, or 167805-301566. In various embodiments, an AON comprises a sequence that shares 98% identity with an equal length portion of any one of SEQ ID NOs: 1-954, 1914-149354, 149362-158581, or 167805-301566.

In some embodiments, the spacer is of Formula (X):

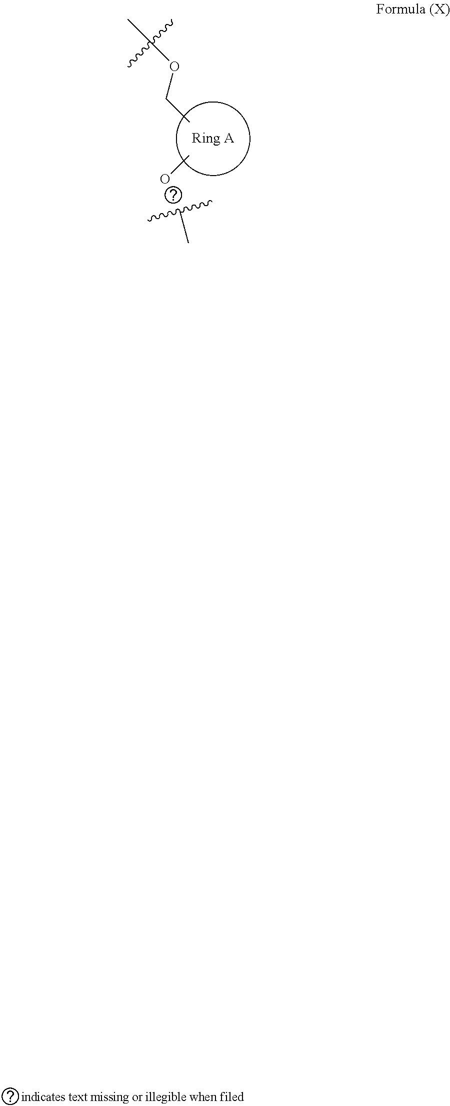

wherein ring A is as defined herein.

In some embodiments, the spacer is of Formula (Xa):

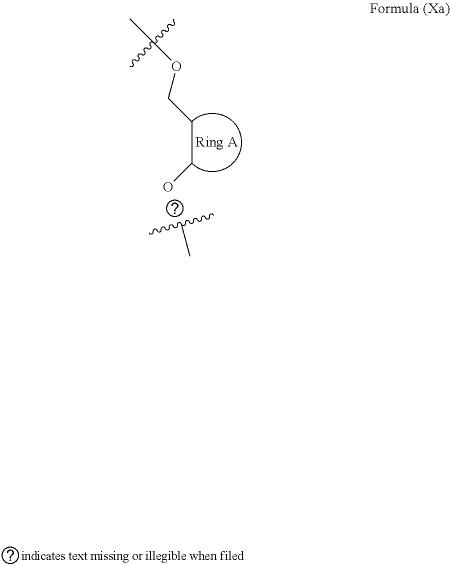

wherein ring A is as defined herein and the —CH₂—O— group is on a ring A atom adjacent to the —O— group.

As generally defined herein, ring A of formulae (X) and (Xa), is an optionally substituted 4-8 member monocyclic cycloalkyl group (e.g. ring A is cyclobutyl, cyclopentyl, cyclohexyl, cycloheptyl, cyclooctyl) or a 4-8 member monocyclic heterocyclyl group, wherein the heterocyclyl group contains 1 or 2 heteroatoms selected from O, S and N (e.g. ring A is oxetanyl, tetrahydrofuranyl, tetrahydropyranyl, 1,4-dioxanyl, pyrrolidinyl, piperidinyl, piperazinyl, morpholinyl, azepanyl). In some embodiments, ring A is tetrahydrofuranyl. In some embodiments, ring A is tetrahydropyranyl. In some embodiments, ring A is pyrrolidinyl. In some embodiments, ring A is cyclopentyl. In some embodiments, the monocyclic cycloalkyl or monocyclic heterocyclyl is not further substituted. In some embodiments, the cycloalkyl or heterocyclyl is further substituted with 0, 1, 2 or 3 substituents selected from halo (e.g., —F, —Cl), —OMe, —OEt —O(CH₂)OMe, —O(CH₂)₂OMe and CN. In some embodiments, the spacer is represented by Formula (I), wherein:

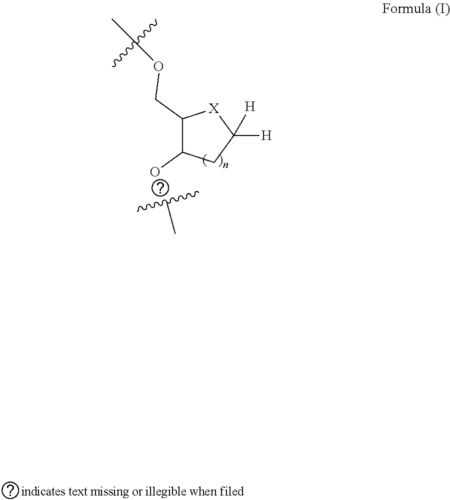

X is selected from —CH₂— and —O—; and
n is 0, 1, 2 or 3.

In some embodiments, the spacer is represented by Formula (I′), wherein:

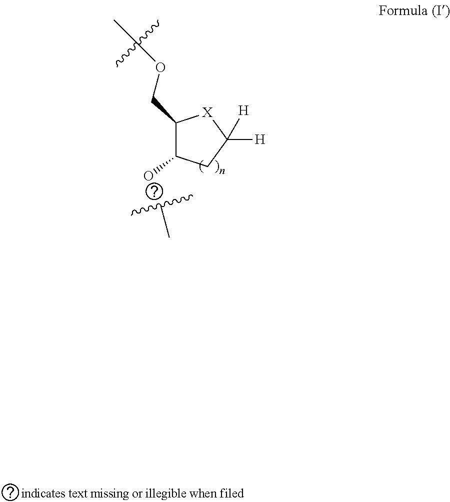

X is selected from —CH₂— and —O—; and
n is 0, 1, 2 or 3.

In some embodiments, the spacer is represented by Formula (Ia), wherein:

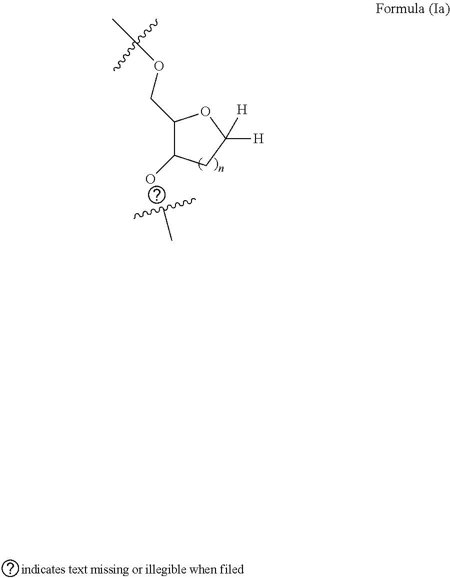

and n is 0, 1, 2 or 3.

In some embodiments, the spacer is represented by Formula (Ia′), wherein:

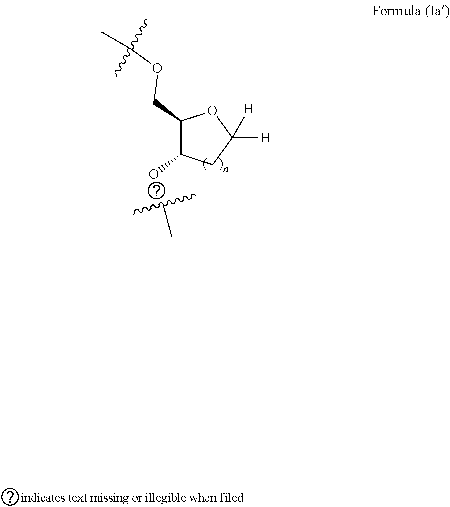

and n is 0, 1, 2 or 3.

As generally defined herein, X is selected from —CH₂— and —O—. In some embodiments, X is —CH₂—. In other embodiments, X is —O—.

As generally defined herein, n is 0, 1, 2 or 3. In some embodiments, n is 0. In some embodiments, n is 1 or 2. In some embodiments, n is 1. In other embodiments, n is 2. In certain embodiments, n is 3.

In some embodiments, the spacer is represented by Formula (II), wherein:

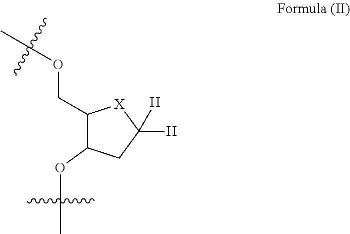

X is selected from —CH₂— and —O—.

In some embodiments, the spacer is represented by Formula (II′), wherein:

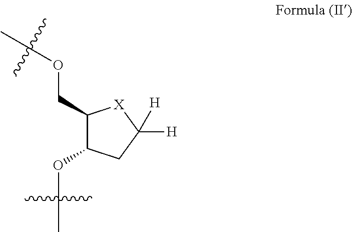

X is selected from —CH₂— and —O—.

In some embodiments, the spacer is represented by Formula (Iia), wherein:

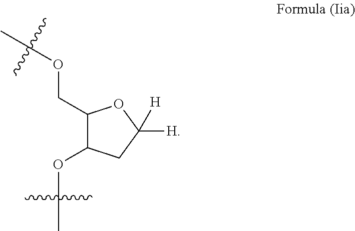

In some embodiments, the spacer is represented by Formula (Iia′), wherein:

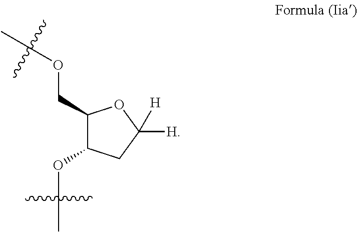

In some embodiments, the spacer is represented by Formula (III), wherein:

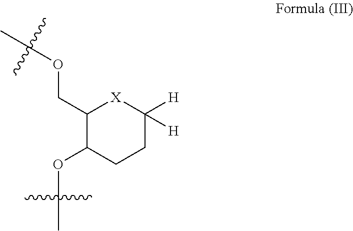

X is selected from —CH₂— and —O—.

In some embodiments, the spacer is represented by Formula (III′), wherein:

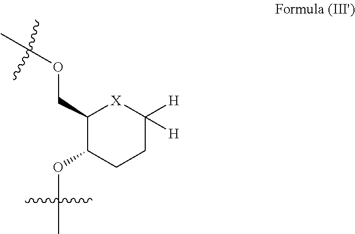

X is selected from —CH₂— and —O—.

In some embodiments, the spacer is represented by Formula (IIIa), wherein:

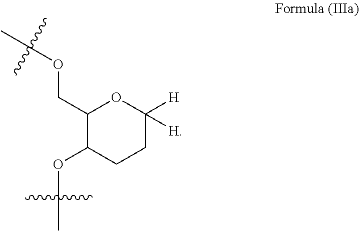

In some embodiments, the spacer is represented by Formula (IIIa′), wherein:

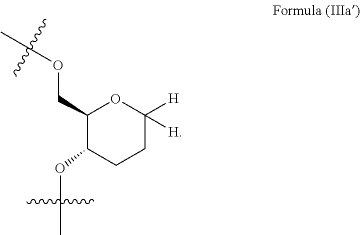

In some embodiments, the open positions of Formulae (I), (I′), (Ia), (Ia′), (II), (II′), (Iia), (Iia′), (III), (III′), (IIIa) and (IIIa′) (i.e., the positions not specifically depicted as bearing exclusively hydrogen atoms, including the —CH₂— group of X) are further substituted with 0-3 substituents selected from halo (e.g., —F, —Cl), —OMe, —OEt —O(CH₂)OMe, —O(CH₂)₂OMe and CN. In some embodiments, Formulae (I), (I′), (Ia), (Ia′), (II), (II′), (Iia), (Iia′), (III), (III′), (IIIa) and (IIIa′) are not further substituted.

As described further below, an AON with one or more spacers is described in reference to a corresponding parent oligonucleotide, to a corresponding oligonucleotide variant, or to a corresponding gapmer oligonucleotide. In various embodiments, an oligonucleotide with one or more spacers differs from a parent oligonucleotide, an oligonucleotide variant, or a gapmer oligonucleotide in that each of the one or more spacers replaces a nucleoside in parent oligonucleotide, oligonucleotide variant, or gapmer oligonucleotide. As used hereafter, the “position” of the oligonucleotide refers to a particular location as counted from the 5′ end of the oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the parent oligonucleotide, of the oligonucleotide variant, or of the gapmer oligonucleotide. In particular embodiments, a spacer replaces a nucleoside at position 7 of the parent oligonucleotide, of the oligonucleotide variant, or of the gapmer oligonucleotide. In particular embodiments, a spacer replaces a nucleoside at position 8 of the parent oligonucleotide, of the oligonucleotide variant, or of the gapmer oligonucleotide. In particular embodiments, a spacer replaces a nucleoside at position 11 of the parent oligonucleotide, of the oligonucleotide variant, or of the gapmer oligonucleotide. In particular embodiments, a spacer replaces a nucleoside at position 14 of the parent oligonucleotide, of the oligonucleotide variant, or of the gapmer oligonucleotide. In particular embodiments, a spacer replaces a nucleoside at position 16 of the parent oligonucleotide, of the oligonucleotide variant, or of the gapmer oligonucleotide. In particular embodiments, a spacer replaces a nucleoside at position 19 of the parent oligonucleotide, of the oligonucleotide variant, or of the gapmer oligonucleotide. In particular embodiments, a spacer replaces a nucleoside at position 22 of the oligonucleotide variant or of the gapmer oligonucleotide.

In various embodiments, an oligonucleotide includes one spacer that replaces a nucleoside in the parent oligonucleotide, oligonucleotide variant, or gapmer oligonucleotide (e.g., one spacer replaces one nucleoside of the parent oligonucleotide, oligonucleotide variant, or gapmer oligonucleotide). Example oligonucleotides with one spacer are shown below in Table 4A. In particular embodiments, the spacer replaces a nucleoside between positions 9 and 15 of the parent oligonucleotide, oligonucleotide variant, or gapmer oligonucleotide. In particular embodiments, the spacer replaces a nucleoside between positions 9 and 12 of the parent oligonucleotide, oligonucleotide variant, or gapmer oligonucleotide. In particular embodiments, the spacer replaces a nucleoside at position 9 of the parent oligonucleotide, oligonucleotide variant, or gapmer oligonucleotide. In particular embodiments, the spacer replaces a nucleoside at position 10 of the parent oligonucleotide, oligonucleotide variant, or gapmer oligonucleotide. In particular embodiments, the spacer replaces a nucleoside at position 11 of the parent oligonucleotide, oligonucleotide variant, or gapmer oligonucleotide. In particular embodiments, the spacer replaces a nucleoside at position 12 of the parent oligonucleotide, oligonucleotide variant, or gapmer oligonucleotide. In particular embodiments, the spacer replaces a nucleoside between positions 12 and 16 of the parent oligonucleotide, oligonucleotide variant, or gapmer oligonucleotide. In particular embodiments, the spacer replaces a nucleoside at position 15 of the parent oligonucleotide, oligonucleotide variant, or gapmer oligonucleotide.

In various embodiments, an oligonucleotide including one spacer has 2 segments, where at least one of the 2 segments has at most 11 linked nucleosides. For example, the oligonucleotide may be 23 oligonucleotide units in length, and the spacer can be located at position 12. Therefore, the oligonucleotide has 2 segments divided by the spacer, where both of the 2 segments are 11 nucleosides in length. In various embodiments, an oligonucleotide including one spacer has 2 segments, where at least one of the 2 segments has at most 10 linked nucleosides. For example, the oligonucleotide may be 21 oligonucleotide units in length, and the spacer can be located at position 11. Therefore, the oligonucleotide has 2 segments divided by the spacer, where both of the 2 segments are 10 nucleosides in length. As another example, the oligonucleotide may be 25 oligonucleotide units in length, and the spacer can be located at position 15. Therefore, the oligonucleotide has 2 segments divided by the spacer, where one of the 2 segments is 14 nucleobases in length and the second of the 2 segments is 10 nucleobases in length. As another example, the oligonucleotide may be 20 oligonucleotide units in length, and the spacer can be located at position 11. Therefore, the oligonucleotide has 2 segments divided by the spacer, where one of the 2 segments is 10 nucleobases in length and the second of the 2 segments is 9 nucleobases in length. As another example, the oligonucleotide may be 18 oligonucleotide units in length, and the spacer can be located at position 11. Therefore, the oligonucleotide has 2 segments divided by the spacer, where one of the 2 segments is 10 nucleobases in length and the second of the 2 segments is 7 nucleobases in length. As another example, the oligonucleotide may be 18 oligonucleotide units in length, and the spacer can be located at position 9. Therefore, the oligonucleotide has 2 segments divided by the spacer, where one of the 2 segments is 8 nucleobases in length and the second of the 2 segments is 9 nucleobases in length.

In various embodiments, an oligonucleotide includes two spacers that each replace a nucleoside in the parent oligonucleotide, oligonucleotide variant, or gapmer oligonucleotide (e.g., two spacers replace two separate nucleosides of the parent oligonucleotide, oligonucleotide variant, or gapmer oligonucleotide). Example oligonucleotides with two spacers are shown below in Table 4B and Table 4C. In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the parent oligonucleotide, oligonucleotide variant, or gapmer oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the parent oligonucleotide, oligonucleotide variant, or gapmer oligonucleotide. In particular embodiments, the second spacer replaces a nucleoside between positions 14 and 22 of the parent oligonucleotide, oligonucleotide variant, or gapmer oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the parent oligonucleotide, oligonucleotide variant, or gapmer oligonucleotide.

In preferred embodiments, the first spacer replaces a nucleoside at position 7 of the parent oligonucleotide, oligonucleotide variant, or gapmer oligonucleotide and the second spacer replaces a nucleoside at position 14 of the parent oligonucleotide, oligonucleotide variant, or gapmer oligonucleotide. In preferred embodiments, the first spacer replaces a nucleoside at position 7 of the parent oligonucleotide, oligonucleotide variant, or gapmer oligonucleotide and the second spacer replaces a nucleoside at position 15 of the parent oligonucleotide, oligonucleotide variant, or gapmer oligonucleotide. In preferred embodiments, the first spacer replaces a nucleoside at position 7 of the parent oligonucleotide, oligonucleotide variant, or gapmer oligonucleotide and the second spacer replaces a nucleoside at position 19 of the parent oligonucleotide, oligonucleotide variant, or gapmer oligonucleotide. In preferred embodiments, the first spacer replaces a nucleoside at position 8 of the parent oligonucleotide, oligonucleotide variant, or gapmer oligonucleotide and the second spacer replaces a nucleoside at position 16 of the K parent oligonucleotide, oligonucleotide variant, or gapmer oligonucleotide. In preferred embodiments, the first spacer replaces a nucleoside at position 11 of the parent oligonucleotide, oligonucleotide variant, or gapmer oligonucleotide and the second spacer replaces a nucleoside at position 19 of the parent oligonucleotide, oligonucleotide variant, or gapmer oligonucleotide. In preferred embodiments, the first spacer replaces a nucleoside at position 11 of the parent oligonucleotide, oligonucleotide variant, or gapmer oligonucleotide and the second spacer replaces a nucleoside at position 22 of the parent oligonucleotide, oligonucleotide variant, or gapmer oligonucleotide. In preferred embodiments, the first spacer replaces a nucleoside at position 9 of the parent oligonucleotide, oligonucleotide variant, or gapmer oligonucleotide and the second spacer replaces a nucleoside at position 19 of the parent oligonucleotide, oligonucleotide variant, or gapmer oligonucleotide. In preferred embodiments, the first spacer replaces a nucleoside at position 5 of the parent oligonucleotide, oligonucleotide variant, or gapmer oligonucleotide and the second spacer replaces a nucleoside at position 17 of the parent oligonucleotide, oligonucleotide variant, or gapmer oligonucleotide.

In various embodiments, an oligonucleotide includes three spacers that each replace a nucleoside in the parent oligonucleotide, oligonucleotide variant, or gapmer oligonucleotide (e.g., three spacers replace three separate nucleosides of the parent oligonucleotide, oligonucleotide variant, or gapmer oligonucleotide). In particular embodiments, the first spacer replaces a nucleoside between positions 7 and 11 of the parent oligonucleotide, oligonucleotide variant, or gapmer oligonucleotide. In particular embodiments, the second spacer replaces a nucleoside between positions 14 and 22 of the parent oligonucleotide, oligonucleotide variant, or gapmer oligonucleotide. In particular embodiments, the third spacer replaces a nucleoside between positions 21 and 24 of the parent oligonucleotide, oligonucleotide variant, or gapmer oligonucleotide. In some embodiments, the first spacer replaces a nucleoside between positions 2 and 5 of the parent oligonucleotide, oligonucleotide variant, or gapmer oligonucleotide. In particular embodiments, the second spacer replaces a nucleoside between positions 8 and 12 of the parent oligonucleotide, oligonucleotide variant, or gapmer oligonucleotide. In particular embodiments, the third spacer replaces a nucleoside between positions 18 and 22 of the parent oligonucleotide, oligonucleotide variant, or gapmer oligonucleotide.

In various embodiments, the three spacers in an oligonucleotide are positioned such that each of the four segments of the oligonucleotide are at most 7 linked nucleosides in length. For example, an oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the oligonucleotide with one or more spacers has a particular GC content. As used herein, GC content (or guanine-cytosine content) is the percentage of nitrogenous bases in the oligonucleotide that are either guanine (G) or cytosine (C). In various embodiments, the oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the oligonucleotide with one or more spacers has at least 30% GC content. In particular embodiments, the oligonucleotide with one or more spacers has at least 40% GC content. In various embodiments, the one or more spacers are positioned in the oligonucleotide to maximize GC content. For example, instead of selecting a guanine or cytosine for replacement by a spacer in the oligonucleotide, a thymine or adenine can be selected for replacement by a spacer.

In various embodiments, an oligonucleotide with spacers is designed such that 1) each segment of the oligonucleotide has at most 7 linked nucleosides and 2) at least two, three, or four spacers are positioned adjacent to a guanine group. In some embodiments, an oligonucleotide with spacers is designed such that 1) each segment of the oligonucleotide has at most 7 linked nucleosides and 2) each of two spacers precede a guanine group.

In various embodiments, the inclusion of one or more spacers in the oligonucleotide does not decrease the effectiveness of the oligonucleotide with the spacers in reducing the frequency of target mRNA or pre-mRNA transcripts in comparison to the effect of a corresponding parent oligonucleotide, oligonucleotide variant, or gapmer oligonucleotide. Example mRNA or pre-mRNa transcripts include gene products of any of PPM1A, ATXN2, SOD1, or MAPT. In various embodiments, the inclusion of one or more spacers in the oligonucleotide increases the effectiveness of the oligonucleotide with the spacers in reducing the frequency of target mRNA or pre-mRNA transcripts in comparison to the effect of a corresponding parent oligonucleotide, oligonucleotide variant, or gapmer oligonucleotide.

Tables 4A, 4B, and 4C document example AONs with one or more spacers and their relation to corresponding parent oligonucleotides, corresponding oligonucleotide variants, and/or corresponding gapmer oligonucleotides. Each oligonucleotide is assigned a sequence name. As used hereafter, the nomenclature of the sequence name is expressed as “X_spA” (for an AON with one spacer), “X_spA_spB” (for an AON with two spacers), or “X_spA_spB_spC” (for an AON with three spacers). Here, “X” refers to the length of the AON, “A” refers to the position in the AON where the first spacer is located, “B” refers to the position in the AON where the second spacer is located, and if present, “C” refers to the position in the AON where the third spacer is located.

Example AONs with one spacer are documented below in Table 4A.

TABLE 4A
Identification of AONs with one spacer. Here, each AON has 2 segments.

| | | Sequence* (where X indicates a |
|---|---|---|
| | Relation to gapmer | nucleoside of the gapmer |
| | oligonucleotide or | oligonucleotide or oligonucleotide |
| | gapmer | gapmer variant and S_{y} indicates |
| | oligonucleotide | presence of a Spacer where y |
| Sequence name | variant | denotes the position) (5′→3′) |
| Gapmer | N/A | XXXXXXXXXXXXXXXXXXXX |
| oligonucleotide (e.g., | | (20 mer) |
| 5-10-5 gapmer) | | |
| Gapmer | Nucleoside at | S₁XXXXXXXXXXXXXXXXXXX |
| oligonucleotide | position 1 of | |
| (20 mer) with Spacer at | 20 mer is | |
| position 1 | substituted with a | |
| (AON 20_sp1) | spacer | |
| Gapmer | Nucleoside at | XS₂XXXXXXXXXXXXXXXXXX |
| oligonucleotide | position 2 of | |
| (20 mer) with Spacer at | 20 mer is | |
| position 2 | substituted with a | |
| (AON 20_sp2) | spacer | |
| Gapmer | Nucleoside at | XXS₃XXXXXXXXXXXXXXXXX |
| oligonucleotide | position 3 of | |
| (20 mer) with Spacer at | 20 mer is | |
| position 3 | substituted with a | |
| (AON 20_sp3) | spacer | |
| Gapmer | Nucleoside at | XXXS₄XXXXXXXXXXXXXXXX |
| oligonucleotide | position 4 of | |
| (20 mer) with Spacer at | 20 mer is | |
| position 4 | substituted with a | |
| (AON 20_sp4) | spacer | |
| Gapmer | Nucleoside at | XXXXS₅XXXXXXXXXXXXXXX |
| oligonucleotide | position 5 of | |
| (20 mer) with Spacer at | 20 mer is | |
| position 5 | substituted with a | |
| (AON 20_sp5) | spacer | |
| Gapmer | Nucleoside at | XXXXXS₆XXXXXXXXXXXXXX |
| oligonucleotide | position 6 of | |
| (20 mer) with Spacer at | 20 mer is | |
| position 6 | substituted with a | |
| (AON 20_sp7) | spacer | |
| Gapmer | Nucleoside at | XXXXXXS₇XXXXXXXXXXXXX |
| oligonucleotide | position 7 of | |
| (20 mer) with Spacer at | 20 mer is | |
| position 7 | substituted with a | |
| (AON 20_sp7) | spacer | |
| Gapmer | Nucleoside at | XXXXXXXS₈XXXXXXXXXXXX |
| oligonucleotide | position 8 of | |
| (20 mer) with Spacer at | 20 mer is | |
| position 8 | substituted with a | |
| (AON 20_sp8) | spacer | |
| Gapmer | Nucleoside at | XXXXXXXXS₉XXXXXXXXXXX |
| oligonucleotide | position 9 of | |
| (20 mer) with Spacer at | 20 mer is | |
| position 9 | substituted with a | |
| (AON 20_sp9) | spacer | |
| Gapmer | Nucleoside at | XXXXXXXXXS₁₀XXXXXXXXXX |
| oligonucleotide | position 10 of | |
| (20 mer) with Spacer at | 20 mer is | |
| position 10 | substituted with a | |
| (AON 20_sp10) | spacer | |
| Gapmer | Nucleoside at | XXXXXXXXXXS₁₁XXXXXXXXX |
| oligonucleotide | position 11 of | |
| (20 mer) with Spacer at | 20 mer is | |
| position 11 | substituted with a | |
| (AON 20_sp11) | spacer | |
| Gapmer | Nucleoside at | XXXXXXXXXXXS₁₂XXXXXXXX |
| oligonucleotide | position 12 of | |
| (20 mer) with Spacer at | 20 mer is | |
| position 12 | substituted with a | |
| (AON 20_sp12) | spacer | |
| Gapmer | Nucleoside at | XXXXXXXXXXXXS₁₃XXXXXXX |
| oligonucleotide | position 13 of | |
| (20 mer) with Spacer at | 20 mer is | |
| position 13 | substituted with a | |
| (AON 20_sp13) | spacer | |
| Gapmer | Nucleoside at | XXXXXXXXXXXXXS₁₄XXXXXX |
| oligonucleotide | position 14 of | |
| (20 mer) with Spacer at | 20 mer is | |
| position 14 | substituted with a | |
| (AON 20_sp14) | spacer | |
| Gapmer | Nucleoside at | XXXXXXXXXXXXXXS₁₅XXXXX |
| oligonucleotide | position 15 of | |
| (20 mer) with Spacer at | 20 mer is | |
| position 15 | substituted with a | |
| (AON 20_sp15) | spacer | |
| Gapmer | Nucleoside at | XXXXXXXXXXXXXXXS₁₆XXXX |
| oligonucleotide | position 16 of | |
| (20 mer) with Spacer at | 20 mer is | |
| position 16 | substituted with a | |
| (AON 20_sp16) | spacer | |
| Gapmer | Nucleoside at | XXXXXXXXXXXXXXXXS₁₇XXX |
| oligonucleotide | position 17 of | |
| (20 mer) with Spacer at | 20 mer is | |
| position 17 | substituted with a | |
| (AON 20_sp17) | spacer | |
| Gapmer | Nucleoside at | XXXXXXXXXXXXXXXXXS₁₈XX |
| oligonucleotide | position 18 of | |
| (20 mer) with Spacer at | 20 mer is | |
| position 18 | substituted with a | |
| (AON 20_sp18) | spacer | |
| Gapmer | Nucleoside at | XXXXXXXXXXXXXXXXXXS₁₉X |
| oligonucleotide | position 19 of | |
| (20 mer) with Spacer at | 20 mer is | |
| position 18 | substituted with a | |
| (AON 20_sp19) | spacer | |
| Gapmer | Nucleoside at | XXXXXXXXXXXXXXXXXXXS₂₀ |
| oligonucleotide | position 20 of | |
| (20 mer) with Spacer at | 20 mer is | |
| position 20 | substituted with a | |
| (AON 20_sp18) | spacer | |
| Gapmer | N/A | XXXXXXXXXXXXXXXXXXXXXXX |
| oligonucleotide variant | | (23 mer) |
| (23 mer) (e.g., 6-11-6 | | |
| gapmer) | | |
| Gapmer | Nucleoside at | XXXXXXXXXXXS₁₂XXXXXXXXXXX |
| oligonucleotide | position 12 of | |
| (23 mer) with Spacer at | 23 mer is | |
| position 12 | substituted with a | |
| (AON 23_sp12) | spacer | |
| Gapmer | N/A | XXXXXXXXXXXXXXXXXXXXX |
| oligonucleotide variant | | (21 mer) |
| (21 mer) | | |
| Gapmer | Nucleoside at | XXXXXXXXXXS₁₁XXXXXXXXXX |
| oligonucleotide | position 11 of | |
| (21 mer) with Spacer at | 21 mer is | |
| position 11 | substituted with a | |
| (AON 21_sp11) | spacer | |
| Gapmer | N/A | XXXXXXXXXXXXXXXXXXX |
| oligonucleotide variant | | (19 mer) |
| (19 mer) | | |
| Gapmer | Nucleoside at | XXXXXXXXXS₁₀XXXXXXXXX |
| oligonucleotide | position 10 of | |
| (19 mer) with Spacer at | 19 mer is | |
| position 10 | substituted with a | |
| (AON 19_sp10) | spacer | |
| Gapmer | N/A | XXXXXXXXXXXXXXXXXX |
| oligonucleotide variant | | (18 mer) |
| (18 mer) (e.g., 4-10-4 | | |
| gapmer) | | |
| Gapmer | Nucleoside at | XXXXXXXXS₉XXXXXXXXX |
| oligonucleotide | position 9 of | |
| (18 mer) with Spacer at | 18 mer is | |
| position 9 | substituted with a | |
| (AON 18_sp9) | spacer | |
| Gapmer | Nucleoside at | XXXXXXXXXS₁₀XXXXXXXX |
| oligonucleotide | position 10 of | |
| (18 mer) with Spacer at | 18 mer is | |
| position 10 | substituted with a | |
| (AON 18_sp10) | spacer | |
| Gapmer | Nucleoside at | XXXXXXXXXXS₁₁XXXXXXX |
| oligonucleotide | position 11 of | |
| (18 mer) with Spacer at | 18 mer is | |
| position 11 | substituted with a | |
| (AON 18_sp11) | spacer | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, oligonucleotides disclosed herein include two spacers. In various embodiments, the inclusion of two spacers divide up the oligonucleotide into three separate segments, where at least one of the segments is at most 7 linked nucleosides in length.

In particular embodiments, a first spacer is located between positions 5 and 11 of the oligonucleotide. In various embodiments, the first spacer is located between positions 7 and 11 of the oligonucleotide. In various embodiments, the second spacer is located between positions 15 and 19 of the oligonucleotide. In various embodiments, the first spacer and the second spacer are separated by at least 5 nucleobases, at least 6 nucleobases, or at least 7 nucleobases in the oligonucleotide.

In various embodiments, the first spacer is located between positions 5 and 11 of the oligonucleotide, and the second spacer is located between positions 15 and 19 of the oligonucleotide. In various embodiments, the first spacer is located at position 8 of the oligonucleotide, and wherein the second spacer is located at position 16 of the oligonucleotide. In various embodiments, the first spacer is located at position 5 of the oligonucleotide, and wherein the second spacer is located at position 17 of the oligonucleotide. In various embodiments, the first spacer is located at position 7 of the oligonucleotide, and wherein the second spacer is located at position 14 of the oligonucleotide. In various embodiments, the first spacer is located at position 7 of the oligonucleotide and wherein the second spacer is located at position 15 of the oligonucleotide. In various embodiments, the first spacer is located at position 11 of the oligonucleotide, and wherein the second spacer is located at position 19 of the oligonucleotide.

Example AONs with two spacers are documented below in Table 4B.

TABLE 4B
Identification of AONs with two spacers. Here, each AON has 3 segments.

| | | Sequence* (where X indicates a |
|---|---|---|
| | | nucleoside of the gapmer |
| | Relation to | oligonucleotide and S_{y} indicates |
| | gapmer | presence of a Spacer where y denotes |
| Sequence name | oligonucleotide | the position) (5′→3′) |
| 20 mer Gapmer | N/A | XXXXXXXXXXXXXXXXXXXX |
| oligonucleotide | | |
| (e.g., 5-10-5 | | |
| gapmer) | | |
| Gapmer | Nucleosides at | XXXXS₅XXXXXXXXXS₁₅XXXXX |
| oligonucleotide | positions 5 and | |
| with Spacers at | 15 are each | |
| positions 5 and 15 | substituted with | |
| (AON 20_sp5sp15) | a spacer | |
| Gapmer | Nucleosides at | XXXXS₅XXXXXXXXXXS₁₆XXXX |
| oligonucleotide | positions 5 and | |
| with Spacers at | 16 are each | |
| positions 5 and 16 | substituted with | |
| (AON 20_sp5sp16) | a spacer | |
| Gapmer | Nucleosides at | XXXXS₅XXXXXXXXXXXS₁₇XXX |
| oligonucleotide | positions 5 and | |
| with Spacers at | 17 are each | |
| positions 5 and 17 | substituted with | |
| (AON 20_sp5sp17) | a spacer | |
| Gapmer | Nucleosides at | XXXXS₅XXXXXXXXXXXXS₁₈XX |
| oligonucleotide | positions 5 and | |
| with Spacers at | 18 are each | |
| positions 5 and 18 | substituted with | |
| (AON 20_sp5sp18) | a spacer | |
| Gapmer | Nucleosides at | XXXXS₅XXXXXXXXXXXXXS₁₉X |
| oligonucleotide | positions 5 and | |
| with Spacers at | 19 are each | |
| positions 5 and 19 | substituted with | |
| (AON 20_sp5sp19) | a spacer | |
| Gapmer | Nucleosides at | XXXXXS₆XXXXXXXXS₁₅XXXXX |
| oligonucleotide | positions 6 and | |
| with Spacers at | 15 are each | |
| positions 6 and 15 | substituted with | |
| (AON 20_sp6sp15) | a spacer | |
| Gapmer | Nucleosides at | XXXXXS₆XXXXXXXXXS₁₆XXXX |
| oligonucleotide | positions 6 and | |
| with Spacers at | 16 are each | |
| positions 6 and 16 | substituted with | |
| (AON 20_sp6sp16) | a spacer | |
| Gapmer | Nucleosides at | XXXXXS₆XXXXXXXXXXS₁₇XXX |
| oligonucleotide | positions 6 and | |
| with Spacers at | 17 are each | |
| positions 6 and 17 | substituted with | |
| (AON 20_sp6sp17) | a spacer | |
| Gapmer | Nucleosides at | XXXXXS₆XXXXXXXXXXXS₁₈XX |
| oligonucleotide | positions 6 and | |
| with Spacers at | 18 are each | |
| positions 6 and 18 | substituted with | |
| (AON 20_sp6sp18) | a spacer | |
| Gapmer | Nucleosides at | XXXXXS₆XXXXXXXXXXXXS₁₉X |
| oligonucleotide | positions 6 and | |
| with Spacers at | 19 are each | |
| positions 6 and 19 | substituted with | |
| (AON 20_sp6sp19) | a spacer | |
| Gapmer | Nucleosides at | XXXXXXS₇XXXXXXS₁₄XXXXXX |
| oligonucleotide | positions 7 and | |
| with Spacers at | 14 are each | |
| positions 7 and 14 | substituted with | |
| (AON 20_sp7sp14) | a spacer | |
| Gapmer | Nucleosides at | XXXXXXS₇XXXXXXXS₁₅XXXXX |
| oligonucleotide | positions 7 and | |
| with Spacers at | 15 are each | |
| positions 7 and 15 | substituted with | |
| (AON 20_sp7sp15) | a spacer | |
| Gapmer | Nucleosides at | XXXXXXS₇XXXXXXXXS₁₆XXXX |
| oligonucleotide | positions 7 and | |
| with Spacers at | 16 are each | |
| positions 7 and 16 | substituted with | |
| (AON 20_sp7sp16) | a spacer | |
| Gapmer | Nucleosides at | XXXXXXS₇XXXXXXXXXS₁₇XXX |
| oligonucleotide | positions 7 and | |
| with Spacers at | 17 are each | |
| positions 7 and 17 | substituted with | |
| (AON 20_sp7sp17) | a spacer | |
| Gapmer | Nucleosides at | XXXXXXS₇XXXXXXXXXXS₁₈XX |
| oligonucleotide | positions 7 and | |
| with Spacers at | 18 are each | |
| positions 7 and 18 | substituted with | |
| (AON 20_sp7sp18) | a spacer | |
| Gapmer | Nucleosides at | XXXXXXS₇XXXXXXXXXXXS₁₉X |
| oligonucleotide | positions 7 and | |
| with Spacers at | 19 are each | |
| positions 7 and 19 | substituted with | |
| (AON 20_sp7sp19) | a spacer | |
| Gapmer | Nucleosides at | XXXXXXXS₈XXXXXXS₁₅XXXXX |
| oligonucleotide | positions 8 and | |
| with Spacers at | 15 are each | |
| positions 8 and 15 | substituted with | |
| (AON 20_sp8sp15) | a spacer | |
| Gapmer | Nucleosides at | XXXXXXXS₈XXXXXXXS₁₆XXXX |
| oligonucleotide | positions 8 and | |
| with Spacers at | 16 are each | |
| positions 8 and 16 | substituted with | |
| (AON 20_sp8sp16) | a spacer | |
| Gapmer | Nucleosides at | XXXXXXXS₈XXXXXXXXS₁₇XXX |
| oligonucleotide | positions 8 and | |
| with Spacers at | 17 are each | |
| positions 8 and 17 | substituted with | |
| (AON 20_sp8sp17) | a spacer | |
| Gapmer | Nucleosides at | XXXXXXXS₈XXXXXXXXXS₁₈XX |
| oligonucleotide | positions 8 and | |
| with Spacers at | 18 are each | |
| positions 8 and 18 | substituted with | |
| (AON 20_sp8sp18) | a spacer | |
| Gapmer | Nucleosides at | XXXXXXXS₈XXXXXXXXXXS₁₉X |
| oligonucleotide | positions 8 and | |
| with Spacers at | 19 are each | |
| positions 8 and 19 | substituted with | |
| (AON 20_sp8sp19) | a spacer | |
| Gapmer | Nucleosides at | XXXXXXXXS₉XXXXXS₁₅XXXXX |
| oligonucleotide | positions 9 and | |
| with Spacers at | 15 are each | |
| positions 9 and 15 | substituted with | |
| (AON 20_sp9sp15) | a spacer | |
| Gapmer | Nucleosides at | XXXXXXXXS₉XXXXXXS₁₆XXXX |
| oligonucleotide | positions 9 and | |
| with Spacers at | 16 are each | |
| positions 9 and 16 | substituted with | |
| (AON 20_sp9sp16) | a spacer | |
| Gapmer | Nucleosides at | XXXXXXXXS₉XXXXXXXS₁₇XXX |
| oligonucleotide | positions 9 and | |
| with Spacers at | 17 are each | |
| positions 9 and 17 | substituted with | |
| (AON 20_sp9sp17) | a spacer | |
| Gapmer | Nucleosides at | XXXXXXXXS₉XXXXXXXXS₁₈XX |
| oligonucleotide | positions 9 and | |
| with Spacers at | 18 are each | |
| positions 9 and 18 | substituted with | |
| (AON 20_sp9sp18) | a spacer | |
| Gapmer | Nucleosides at | XXXXXXXXS₉XXXXXXXXXS₁₉X |
| oligonucleotide | positions 9 and | |
| with Spacers at | 19 are each | |
| positions 9 and 19 | substituted with | |
| (AON 20_sp9sp19) | a spacer | |
| Gapmer | Nucleosides at | XXXXXXXXXS₁₀XXXXS₁₅XXXXX |
| oligonucleotide | positions 10 and | |
| with Spacers at | 15 are each | |
| positions 10 and 15 | substituted with | |
| (AON 20_sp10sp15) | a spacer | |
| Gapmer | Nucleosides at | XXXXXXXXXS₁₀XXXXXS₁₆XXXX |
| oligonucleotide | positions 10 and | |
| with Spacers at | 16 are each | |
| positions 10 and 16 | substituted with | |
| (AON 20_sp10sp16) | a spacer | |
| Gapmer | Nucleosides at | XXXXXXXXXS₁₀XXXXXXS₁₇XXX |
| oligonucleotide | positions 10 and | |
| with Spacers at | 17 are each | |
| positions 10 and 17 | substituted with | |
| (AON 20_sp10sp17) | a spacer | |
| Gapmer | Nucleosides at | XXXXXXXXXS₁₀XXXXXXXS₁₈XX |
| oligonucleotide | positions 10 and | |
| with Spacers at | 18 are each | |
| positions 10 and 18 | substituted with | |
| (AON 20_sp10sp18) | a spacer | |
| Gapmer | Nucleosides at | XXXXXXXXXS₁₀XXXXXXXXS₁₉X |
| oligonucleotide | positions 10 and | |
| with Spacers at | 19 are each | |
| positions 10 and 19 | substituted with | |
| (AON 20_sp10sp19) | a spacer | |
| Gapmer | Nucleosides at | XXXXXXXXXXS₁₁XXXS₁₅XXXXX |
| oligonucleotide | positions 11 and | |
| with Spacers at | 15 are each | |
| positions 11 and 15 | substituted with | |
| (AON 20_spl1sp15) | a spacer | |
| Gapmer | Nucleosides at | XXXXXXXXXXS₁₁XXXXS₁₆XXXX |
| oligonucleotide | positions 11 and | |
| with Spacers at | 16 are each | |
| positions 11 and 16 | substituted with | |
| (AON 20_spl1sp16) | a spacer | |
| Gapmer | Nucleosides at | XXXXXXXXXXS₁₁XXXXXS₁₇XXX |
| oligonucleotide | positions 11 and | |
| with Spacers at | 17 are each | |
| positions 11 and 17 | substituted with | |
| (AON 20_sp11sp17) | a spacer | |
| Gapmer | Nucleosides at | XXXXXXXXXXS₁₁XXXXXXS₁₈XX |
| oligonucleotide | positions 11 and | |
| with Spacers at | 18 are each | |
| positions 11 and 18 | substituted with | |
| (AON 20_spl1sp18) | a spacer | |
| Gapmer | Nucleosides at | XXXXXXXXXXS₁₁XXXXXXXS₁₉X |
| oligonucleotide | positions 11 and | |
| with Spacers at | 19 are each | |
| positions 11 and 19 | substituted with | |
| (AON 20_sp11sp19) | a spacer | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, AONs with one or more spacers are reduced in length or increased in length in comparison to the AONs described above in Tables 4A and 4B. For example, such AONs may be oligonucleotide variants with one or more spacers. In various embodiments, the oligonucleotide variants with one or more spacers are 23mers, 21mers, 19mers, or 18mers. In various embodiments, oligonucleotide variants include two spacers such that the oligonucleotide variant includes three segments that are divided up by the two spacers. In various embodiments, at least one of the three segments has at most 7 linked nucleosides. In various embodiments, each of the three segments has at most 7 linked nucleosides. Example oligonucleotide variants with one or more spacers are shown below in Table 4C.

TABLE 4C
AON variants with two spacers. Here, each AON variant has 3 segments.

| | | Sequence* (where X indicates a |
|---|---|---|
| | | nucleoside of the |
| | | oligonucleotide variant and |
| | Relation to | S_{y} indicates presence of a |
| | oligonucleotide | Spacer where y denotes |
| Sequence name | variant | the position) (5′→3′) |
| Gapmer | N/A | XXXXXXXXXXXXXXXXXXXXXXX |
| oligonucleotide | | (23 mer) |
| variant (23 mer) | | |
| (e.g., 6-11-6 | | |
| gapmer) | | |
| Gapmer | Nucleosides at | XXXXXXXS₈XXXXXXXS₁₆XXXXXXX |
| oligonucleotide | positions 8 and | |
| variant (23 mer) | 16 are | |
| with Spacers at | substituted with | |
| positions 8 and | spacers | |
| 16 (AON | | |
| variant | | |
| 23_sp8sp16) | | |
| Gapmer | N/A | XXXXXXXXXXXXXXXXXXXXX |
| oligonucleotide | | (21 mer) |
| variant (21 mer) | | |
| Gapmer | Nucleosides at | XXXXS₅XXXXXXS₁₂XXXXXXXXX |
| oligonucleotide | positions 5 and | |
| variant (21 mer) | 12 are | |
| with Spacers at | substituted with | |
| positions 5 and | spacers | |
| 12 (AON | | |
| variant | | |
| 21_sp5sp12) | | |
| Gapmer | Nucleosides at | XXXXXXXS₈XXXXXXXS₁₆XXXXX |
| oligonucleotide | positions 8 and | |
| variant (21 mer) | 16 are | |
| with Spacers at | substituted with | |
| positions 8 and | spacers | |
| 16 (AON | | |
| variant | | |
| 21_sp8sp16) | | |
| Gapmer | Nucleosides at | XXXXXS₆XXXXXXXS₁₄XXXXXXX |
| oligonucleotide | positions 6 and | |
| variant (21 mer) | 14 are | |
| with Spacers at | substituted with | |
| positions 6 and | spacers | |
| 14 (AON | | |
| variant | | |
| 21_sp6sp14) | | |
| Gapmer | Nucleosides at | XXXXXXXS₈XXXXXS₁₄XXXXXXX |
| oligonucleotide | positions 8 and | |
| variant (21 mer) | 14 are | |
| with Spacers at | substituted with | |
| positions 8 and | spacers | |
| 14 (AON | | |
| variant | | |
| 21_sp8sp14) | | |
| Gapmer | Nucleosides at | XXXXXS₆XXXXXXXXXXXXXS₂₀X |
| oligonucleotide | positions 6 and | |
| variant (21 mer) | 20 are | |
| with Spacers at | substituted with | |
| positions 8 and | spacers | |
| 14 (AON | | |
| variant | | |
| 21 sp8sp14) | | |
| Gapmer | N/A | XXXXXXXXXXXXXXXXXXX |
| oligonucleotide | | (19 mer) |
| variant (19 mer) | | |
| Gapmer | Nucleosides at | XXXXS₅XXXXXXS₁₂XXXXXXX |
| oligonucleotide | positions 5 and | |
| variant (19 mer) | 12 are | |
| with Spacers at | substituted with | |
| positions 5 and | spacers | |
| 12 (AON | | |
| variant | | |
| 19_sp5sp12) | | |
| Gapmer | Nucleosides at | XXXXXXXS₈XXXXXXS₁₅XXXX |
| oligonucleotide | positions 8 and | |
| variant (19 mer) | 15 are | |
| with Spacers at | substituted with | |
| positions 8 and | spacers | |
| 15 (AON | | |
| variant | | |
| 19_sp8sp15) | | |
| Gapmer | N/A | XXXXXXXXXXXXXXXXXX |
| oligonucleotide | | (18 mer) |
| variant (18 mer) | | |
| (e.g., 5-8-5 | | |
| gapmer) | | |
| Gapmer | Nucleosides at | XXXXS₅XXXXXXS₁₂XXXXXX |
| oligonucleotide | positions 5 and | |
| variant (18 mer) | 12 are | |
| with Spacers at | substituted with | |
| positions 5 and | spacers | |
| 12 (AON | | |
| variant | | |
| 18_sp5sp12) | | |
| Gapmer | Nucleosides at | XXXXXXXS₈XXXXXXS₁₅XXX |
| oligonucleotide | positions 8 and | |
| variant (18 mer) | 15 are | |
| with Spacers at | substituted with | |
| positions 8 and | spacers | |
| 15 (AON | | |
| variant | | |
| 18_sp8sp15) | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, antisense oligonucleotides disclosed herein (e.g., PPM1A AONs, ATXN2 AONs, SOD1 AONs, or MAPT AONs) comprise one or more locked nucleic acids (LNAs). In particular embodiments, an antisense oligonucleotide includes one LNA. In particular embodiments, an antisense oligonucleotide includes two LNAs. In particular embodiments, an antisense oligonucleotide includes three LNAs. Generally, a LNA refers to nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar.

In some embodiments, antisense oligonucleotides disclosed herein (e.g., PPM1A AONs, ATXN2 AONs, SOD1 AONs, or MAPT AONs) comprise one or more spacers as well as one or more locked nucleic acids (LNAs). In some embodiments, antisense oligonucleotides disclosed herein (e.g., PPM1A AONs, ATXN2 AONs, SOD1 AONs, or MAPT AONs) comprise two spacers and two LNAs. In some embodiments, an antisense oligonucleotide disclosed herein comprises two spacers and three LNAs.

In various embodiments, a spacer and a LNA are located adjacent to one another in an antisense oligonucleotide. For example, a spacer can be located at position M of the antisense oligonucleotide. Additionally, a LNA can be located at a position M+1 or position M−1 of the antisense oligonucleotide. In various embodiments, a first spacer is located adjacent to a first LNA and a second spacer is located adjacent to a second LNA in an antisense oligonucleotide. For example, a first spacer can be located at position M of the antisense oligonucleotide and a second spacer can be located at position N of the antisense oligonucleotide. A first LNA can be located at a position M+1 or position M−1 of the antisense oligonucleotide and a second LNA can be located at a position N+1 or position N−1 of the antisense oligonucleotide. In other embodiments, one or more spacers and one or more LNAs are not located adjacent to one another in an antisense oligonucleotide. For example, there may be one, two, three, four, five, six, seven, eight, nine, or ten oligonucleotide units between a spacer and a LNA in an antisense oligonucleotide.

### Chemical Modifications to AONs

As described herein, AONs, such as AONs with a sequence of any one SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, may include one or more chemical modifications to one or more nucleosides and/or to one or more internucleoside linkages. A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to AONs encompass substitutions or changes to internucleoside linkages and/or nucleosides (e.g., sugar moieties or nucleobases of nucleosides). Modified AONs can be preferred over native forms because of desirable properties such as, for example, enhanced cellular uptake, enhanced affinity for nucleic acid target, increased stability in the presence of nucleases, or increased inhibitory activity. Chemically modified nucleosides, nucleobases, and internucleoside linkages are described in Agrawal and Gait, History and Development of Nucleotide Analogues in Nucleic Acids Drugs, in Drug Discovery Series No. 68, Advances in Nucleic Acid Therapeutics, 1-21 (Agrawal and Gait eds., 2019), the contents of which are incorporated by reference herein.

### Modified Internucleoside Linkages

In various embodiments, AONs, such as AONs comprising a sequence of any one SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C), include one or more modified internucleoside linkages. The naturally occurring internucleoside linkage of RNA and DNA is a 3′ to 5′ phosphodiester linkage. AONs having one or more modified, i.e., non-naturally occurring, internucleoside linkages can be selected over antisense compounds having naturally occurring internucleoside linkages because of desirable properties such as, for example, enhanced cellular uptake, enhanced affinity for target nucleic acids, and increased stability in the presence of nucleases.

Modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known. Examples of modified internucleoside linkages include any one of a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, AONs include one or more modified internucleoside linkages that link the oligonucleotide units. In various embodiments, AONs include one, two, three, four, five, six, seven, eight, nine, ten, eleven, twelve, thirteen, fourteen, fifteen, sixteen, seventeen, eighteen, or nineteen modified internucleoside linkages that link the oligonucleotide units.

In various embodiments, each modified internucleoside linkage of the AON can be designed independent of other modified internucleoside linkages of the AON. In other words, the modified internucleoside linkages of an AON need not all be the same type of modified internucleoside linkage. In various embodiments, the modified internucleoside linkages are interspersed throughout the antisense compound.

In various embodiments, AONs include at least one phosphorothioate linkage. In various embodiments, AONs include at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or at least nineteen phosphorothioate linkages. In particular embodiments, AONs include thirteen, fourteen, fifteen, sixteen, seventeen, eighteen, nineteen, twenty, twenty one, or twenty two phosphorothioate linkages. In particular embodiments, AONs include 17 phosphorothioate linkages. In particular embodiments, AONs include 19 phosphorothioate linkages. In particular embodiments, AONs include 22 phosphorothioate linkages. In particular embodiments, all internucleoside linkages of the AON are phosphorothioate linkages.

In various embodiments, an AON includes a mixture of modified internucleoside linkages and naturally occurring phosphodiester linkages. For example, an AON includes at least one phosphodiester linkage and at least one phosphorothioate linkage. In various embodiments, an AON includes between 6 and 10, between 6 and 9, between 6 and 8, between 7 and 10, between 7 and 9, or 6, 7, or 8 phosphorothioate linkages. In some embodiments, an AON includes 6, 7, 8, 9, or 10 phosphorothioate linkages. In some embodiments, an AON includes between 6 and 10, between 6 and 9, between 6 and 8, between 7 and 10, between 7 and 9, or 6, 7, or 8 phosphodiester linkages. In some embodiments, an AON includes 6, 7, 8, 9, or 10 phosphodiester linkages.

In particular embodiments, an AON includes 10 phosphorothioate linkages and 9 phosphodiester linkages. In particular embodiments, an AON includes 6 phosphorothioate linkages and 7 phosphodiester linkages. In particular embodiments, an AON includes 6 phosphorothioate linkages and 9 phosphodiester linkages. In particular embodiments, an AON includes 8 phosphorothioate linkages and 9 phosphodiester linkages. In particular embodiments, an AON includes 8 phosphorothioate linkages and 7 phosphodiester linkages. In particular embodiments, an AON includes 12 phosphorothioate linkages and 7 phosphodiester linkages. In particular embodiments, an AON includes 15 phosphorothioate linkages and 4 phosphodiester linkages. In particular embodiments, an AON includes 15 phosphorothioate linkages and 2 phosphodiester linkages. In particular embodiments, an AON includes 17 phosphorothioate linkages and 2 phosphodiester linkages.

In some embodiments, AONs include internucleoside linkages that are designed according to the gapmer design of the AON. In some embodiments, the 5′ wing region includes at least one modified internucleoside linkage (e.g., modified from the naturally occurring internucleoside linkage of a 3′ to 5′ phosphodiester linkage). In some embodiments, the 5′ wing region includes at least two, at least three, at least four, at least five, at least six, at least seven, at least eight, at least nine, or at least ten modified internucleoside linkages. In some embodiments, the 3′ wing region includes at least one modified internucleoside linkage. In some embodiments, the 3′ wing region includes at least two, at least three, at least four, at least five, at least six, at least seven, at least eight, at least nine, or at least ten modified internucleoside linkages. In some embodiments, the central region includes at least one modified internucleoside linkage. In some embodiments, the central region includes at least two, at least three, at least four, at least five, at least six, at least seven, at least eight, at least nine, or at least ten modified internucleoside linkages.

In particular embodiments all internucleoside linkages of the 5′ wing region are modified internucleoside linkages, such as phosphorothioate linkages. In particular embodiments all internucleoside linkages of the 3′ wing region are modified internucleoside linkages, such as phosphorothioate linkages. In particular embodiments all internucleoside linkages of the central region are modified internucleoside linkages, such as phosphorothioate linkages. In particular embodiments all internucleoside linkages of each of the 5′ wing region, 3′ wing region, and the central region are modified internucleoside linkages, such as phosphorothioate linkages.

In various embodiments, two internucleoside linkages of the 5′ wing region are phosphorothioate linkages. In various embodiments, two internucleoside linkages of the 5′ wing region are phosphodiester linkages. In various embodiments, two internucleoside linkages of the 5′ wing region are phosphorothioate linkages and another two internucleoside linkages of the 5′ wing region are phosphodiester linkages. In various embodiments, two internucleoside linkages of the 3′ wing region are phosphorothioate linkages. In various embodiments, two internucleoside linkages of the 3′ wing region are phosphodiester linkages. In various embodiments, two internucleoside linkages of the 3′ wing region are phosphorothioate linkages and another two internucleoside linkages of the 3′ wing region are phosphodiester linkages.

In various embodiments, four internucleoside linkages of the 5′ wing region are phosphorothioate linkages. In various embodiments, four internucleoside linkages of the 5′ wing region are phosphorothioate linkages and two internucleoside linkages of the 3′ wing region are phosphorothioate linkages. In various embodiments, one internucleoside linkage of the 5′ wing region is a phosphorothioate linkage. In various embodiments, one internucleoside linkage of the 3′ wing region is a phosphorothioate linkage. In various embodiments, one internucleoside linkage of the 5′ wing region is a phosphorothioate linkage and one internucleoside linkage of the 3′ wing region is a phosphorothioate linkage.

In some embodiments, the one or more modified internucleoside linkages in the 5′ wing region, 3′ wing region, or the central region are phosphorothioate internucleoside linkages. In some embodiments, the phosphorothioate linkages are stereochemically pure phosphorothioate linkages. In some embodiments the phosphorothioate linkages are Sp phosphorothioate linkages. In other embodiments, the phosphorothioate linkages are Rp phosphorothioate linkages.

In some embodiments, the one or more modified internucleoside linkages in the 5′ wing region, 3′ wing region, or the central region can be any of an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In various embodiments, each modified internucleoside linkage of the 5′ wing region, 3′ wing region, or the central region can be designed independent of other modified internucleoside linkages. In other words, the modified internucleoside linkages of 5′ wing region, 3′ wing region, and the central region need not all be the same type of modified internucleoside linkage. In various embodiments, modified internucleoside linkages are interspersed throughout the antisense compound.

In various embodiments, one or more internucleoside linkages of the 5′ wing region, the 3′ wing region, or the central region are naturally occurring linkages (e.g., phosphodiester bonds). In various embodiments, all internucleoside linkages of the central region are unmodified internucleoside linkages (e.g., phosphodiester linkages).

In various embodiments, the internucleoside linkages of the one region (e.g., 5′ wing region, 3′ wing region, or the central region) may differ from the internucleoside linkages of another region. In particular embodiments, the 5′ wing region includes at least one modified internucleoside linkage, the 3′ wing region includes at least one modified internucleoside linkage, and all internucleoside linkages of the central region are unmodified internucleoside linkages (e.g., phosphodiester linkages). In some embodiments, the central region of the oligonucleotide comprises phosphodiester bonds and the 5′ wing region and 3′ wing region each comprises one or more phosphorothioate linkages. In particular embodiments, all internucleoside linkages of the 5′ wing region are modified internucleoside linkages, all internucleoside linkages of the 3′ wing region are modified internucleoside linkages, and all internucleoside linkages of the central region are unmodified internucleoside linkages (e.g., phosphodiester linkages).

In particular embodiments, the gapmer AON is a 5-10-5 gapmer and the internucleoside linkages of the gapmer AON are denoted as: sssssssssssssssssss (where “s” refers to a phosphorothioate bond). In particular embodiments, the gapmer AON is a 5-10-5 gapmer and the internucleoside linkages of the gapmer AON are denoted as any of: sssssssssssssssssss, sosssssssssssssosss, sssssooooooooosssss, sososssssssssssosos, soooossssssssssooos, soooossssssssssooss, ooooosssssssssooooo, oooooooooooooosssss, soosssssssssssssoos, soossssssssssssssss, ssssssssssssssssoos, and sssssoooooooooooooo (where “s” refers to a phosphorothioate bond and “o” refers to a phosphodiester bond). In particular embodiments, the gapmer AON is a 3-8-3 gapmer and the internucleoside linkages of the gapmer AON are denoted as: sssssssssssss (where “s” refers to a phosphorothioate bond).

In particular embodiments, the gapmer AON is a 3-8-3 gapmer and the internucleoside linkages of the gapmer AON are denoted as any of: sssooooooosss, ooosssssssooo, ssssssssssooo, sosssssssssos, sosssssssssss, sssssssssssos, and ooossssssssss (where “s” refers to a phosphorothioate bond and “o” refers to a phosphodiester bond). In particular embodiments, the gapmer AON is a 3-10-3 gapmer and the internucleoside linkages of the gapmer AON are denoted as: sssssssssssssss (where “s” refers to a phosphorothioate bond).

In particular embodiments, the gapmer AON is a 3-10-3 gapmer and the internucleoside linkages of the gapmer AON are denoted as any of sssooooooooosss, ooosssssssssooo, ssssssssssssooo, sosssssssssssos, sosssssssssssss, sssssssssssssos, and ooossssssssssss (where “s” refers to a phosphorothioate bond and “o” refers to a phosphodiester bond). In particular embodiments, the gapmer AON is a 4-10-4 gapmer and the internucleoside linkages of the gapmer AON are denoted as: sssssssssssssssss (where “s” refers to a phosphorothioate bond).

In particular embodiments, the gapmer AON is a 4-10-4 gapmer and the internucleoside linkages of the gapmer AON are denoted as any of ssssooooooooossss, oooosssssssssoooo, sssssssssssssoooo, soosssssssssssoos, soossssssssssssss, ssssssssssssssoos, and oooosssssssssssss (where “s” refers to a phosphorothioate bond and “o” refers to a phosphodiester bond). In particular embodiments, the gapmer AON is a 4-8-4 gapmer and the internucleoside linkages of the gapmer AON are denoted as: sssssssssssssss (where “s” refers to a phosphorothioate bond).

In particular embodiments, the gapmer AON is a 4-8-4 gapmer and the internucleoside linkages of the gapmer AON are denoted as any of ssssooooooossss, oooosssssssoooo, sssssssssssoooo, soosssssssssoos, soossssssssssss, ssssssssssssoos, and oooosssssssssss (where “s” refers to a phosphorothioate bond and “o” refers to a phosphodiester bond).

In particular embodiments, the gapmer AON is a 5-8-5 gapmer and the internucleoside linkages of the gapmer AON are denoted as any of: sssssssssssssssss or sossssssssssssoss (where “s” refers to a phosphorothioate bond and “o” refers to a phosphodiester bond).

### Modified Sugar Moieties

AONs, such as AONs with a sequence of any one SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C), can contain one or more nucleosides wherein the sugar group has been modified. Such sugar modified nucleosides may impart enhanced nuclease stability, increased binding affinity, or some other beneficial biological property to the antisense compounds.

In various embodiments, nucleosides with a modified sugar moiety include a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene), a 2′-O-methyl (2′-OMe) nucleoside, 2′-O-(2-methoxyethyl) (2′-MOE) nucleoside, peptide nucleic acid (PNA), bicyclic nucleic acid (BNA), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, morpholino oligomer, tcDNA, 2′-O, 4′-C-ethylene linked nucleic acid (ENA), hexitol nucleic acids (HNA), and tricyclic analog (e.g., tcDNA).

In certain embodiments, nucleosides comprise chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S or CF₂ with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4-S, 2′-F, 2′-OCH₃, 2′-OCH₂CH₃, 2′-O CH₂ CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂S CH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ)—, where each Rₗ, Rₘ and Rₙ is, independently, H or substituted or unsubstituted C₁-C₁₀ alkyl.

Additional examples of modified sugar moieties include a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt) (4′-CH(CH₃)—O-2′), S-constrained ethyl (S-cEt) 2′-4′-bridged nucleic acid, 4′-CH₂—O—CH₂-2′, 4′-CH₂—N(R)-2′, 4′-CH(CH₂OCH₃)—O-2′ (“constrained MOE” or “cMOE”), hexitol nucleic acids (HNA), and tricyclic analog (e.g., tcDNA).

In some embodiments, AONs comprise a 2′-O-methyl nucleoside (2′OMe) (e.g., an AON comprising one or more 2′OMe modified sugar), 2′-O-(2-methoxyethyl) (2′-MOE) (e.g., an AON comprising one or more 2′-MOE modified sugar (e.g., 2′-MOE)), peptide nucleic acid (PNA) (e.g., an AON comprising one or more N-(2-aminoethyl)-glycine units linked by amide bonds or carbonyl methylene linkage as repeating units in place of a sugar-phosphate backbone), locked nucleic acid (LNA) (e.g., an AON comprising one or more locked ribose, and can be a mixture of 2′-deoxy nucleotides or 2′OMe nucleotides), constrained ethyl 2′-4′-bridged nucleic acid (c-ET) (e.g., an AON comprising one or more cET sugar), cMOE (e.g., an AON comprising one or more cMOE sugar), morpholino oligomer (e.g., an AON comprising a backbone comprising one or more PMO), deoxy-2′-fluoro nucleoside (e.g., an AON comprising one or more 2′-fluoro-β-D-arabinonucleoside), 2′-0,4′-C-ethylene linked nucleic acid (ENA) (e.g., an AON comprising one or more ENA modified sugar), hexitol nucleic acid (HNA) (e.g., an AON comprising one or more HNA modified sugar), or tricyclic analog (tcDNA) (e.g., an AON comprising one or more tcDNA modified sugar).

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof (see U.S. Pat. No. 7,399,845, issued on Jul. 15, 2008)); 4′-C(CH₃)(CH₃)—O-2′ (and analogs thereof (see published International Application WO/2009/006478, published Jan. 8, 2009)); 4′-CH₂—N(OCH₃)-2′ (and analogs thereof (see published International Application WO/2008/150729, published Dec. 11, 2008)); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof (see published International Application WO 2008/154401, published on Dec. 8, 2008)).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. No. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[—[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl, each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁).

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-methyleneoxy (4′-CH₂—O-2′) BNA, β-D-methyleneoxy (4′-CH₂—O-2′) BNA, ethyleneoxy (4′-(CH₂)₂—O-2) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA, methylene-thio (4′-CH₂—S-2′) BNA, methylene-amino (4′-CH₂—N(R)-2′) BNA, methyl carbocyclic (4′-CH₂—CH(CH₃)-2′) BNA, and propylene carbocyclic (4′-(CH₂)₃-2′) BNA.

As used herein, “locked nucleic acid” or “LNA” or “LNA nucleosides” refer to modified nucleosides having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA. (D) Aminooxy (4′-CH₂—O—N(R)-2′) LNA and (E) Oxyamino (4′-CH₂—N(R)—O-2′) LNA; wherein R is H, C₁-C₁₂ alkyl, or a protecting group (see U.S. Pat. No. 7,427,672, issued on Sep. 23, 2008).

As used herein, LNA nucleosides include, but are not limited to, nucleosides having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′- bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

In various embodiments, AONs disclosed herein include one or more 2′-O-(2-methoxyethyl) (2′-MOE) nucleosides. In various embodiments, AONs disclosed herein include two, three, four, five, six, seven, eight, nine, ten, eleven, twelve, thirteen, fourteen, fifteen, sixteen, seventeen, eighteen, nineteen, twenty, twenty one, twenty two, twenty three, twenty four, or twenty five 2′-O-(2-methoxyethyl) (2′-MOE) nucleosides. In various embodiments, AONs disclosed herein include more than twenty five 2′-O-(2-methoxyethyl) (2′-MOE) nucleosides.

In some embodiments, AONs include modified sugar moieties that are designed according to the gapmer design of the gapmer AON. In various embodiments, gapmer AONs include one or more modified sugar moieties. In various embodiments, the 5′ wing region includes at least one modified sugar moiety. In various embodiments, the 3′ wing region includes at least one modified sugar moiety. In various embodiments, the 5′ wing region includes at least two, at least three, at least four, at least five, at least six, at least seven, at least eight, at least nine, or at least ten modified sugar moieties. In various embodiments, the 3′ wing region includes at least two, at least three, at least four, at least five, at least six, at least seven, at least eight, at least nine, or at least ten modified sugar moieties. In some embodiments, each of the 5′ wing region and/or the 3′ wing region includes from 1 to 7 modified sugar moieties, such as from two to six modified sugar moieties, from two to five modified sugar moieties, from two to four modified sugar moieties, or from one to three modified sugar moieties. In particular embodiments, the 5′ wing region includes 3 modified sugar moieties and the 3′ wing region includes 3 modified sugar moieties. In particular embodiments, the 5′ wing region includes 4 modified sugar moieties and the 3′ wing region includes 4 modified sugar moieties. In particular embodiments, the 5′ wing region includes 5 modified sugar moieties and the 3′ wing region includes 5 modified sugar moieties.

In various embodiments, the nucleosides with a modified sugar moiety in the 5′ and 3′ wing regions are any one of a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene), a 2′-O-methyl (2′-OMe) nucleoside, 2′-O-(2-methoxyethyl) (2′-MOE) nucleoside, peptide nucleic acid (PNA), bicyclic nucleic acid (BNA), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, morpholino oligomer, tcDNA, 2′-O,4′-C-ethylene linked nucleic acid (ENA), hexitol nucleic acids (HNA), and tricyclic analog (e.g., tcDNA).

In some embodiments, the 5′ wing region and/or 3′ wing region comprises at least one 2′-MOE nucleoside. In some embodiments both the 5′ and 3′ wing regions comprise at least one 2′-MOE nucleoside. In some embodiments, each of the 5′ wing region and the 3′ wing region comprises two, three, four, five, six, seven, eight, nine, or ten 2′-MOE nucleosides. In particular embodiments, each of the 5′ wing region and the 3′ wing region comprises four 2′-MOE nucleosides. In particular embodiments, each of the 5′ wing region and the 3′ wing region comprises five 2′-MOE nucleosides. In some embodiments, all the nucleosides in each of the 5′ wing region and the 3′ wing region are 2′-MOE nucleosides.

In other embodiments, the wing regions may comprise both 2′-MOE nucleosides and other nucleosides (mixed wings), such as DNA nucleosides and/or non-MOE modified nucleosides, such as bicyclic nucleosides (BNAs) (e.g., locked nucleic acid (LNA) nucleosides or constrained ethyl 2′-4′-bridged nucleic acid (cEt) nucleosides), 2′-O-methyl nucleosides, tricycloDNA, S-cEt, morpholinos, or other 2′ substituted nucleosides.

In some embodiments, the 5′ wing region or the 3′ wing region comprises at least one BNA (e.g., at least one LNA nucleoside or cET nucleoside). In some embodiments each of the 5′ and 3′ wing regions comprises a BNA. In some embodiments all the nucleosides in the 5′ and 3′ wing regions are BNAs. In a further embodiment, the BNAs in the 5′ and/or 3′ wing regions are independently selected from the group comprising oxy-LNA, thio-LNA, amino-LNA, cET, and/or ENA, in either the beta-D or alpha-L configurations or combinations thereof.

In some embodiments, the 5′ and/or 3′ wing comprises at least one 2′-O-methyl nucleoside. In some embodiments, the 5′ wing comprises at least one 2′-O-methyl nucleoside. In some embodiments both the 5′ and 3′ wing regions comprise a 2′-O-methyl nucleoside. In some embodiments all the nucleosides in the wing regions are 2′-O-methyl nucleosides.

### Modified Nucleobase

AONs, such as AONs with a sequence of any one SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C), include one or more modified nucleobases. Examples of modified nucleobases, including a 5-methylpyrimidine, for example, 5-methylcytosine or 5-methoxyuridine, a 5-methylpurine, for example, 5-methylguanine, or pseudouridine.

In various embodiments, an AON includes at least one modified nucleobase. In various embodiments, an AON includes two, three, four, five, six, seven, eight, nine, or ten modified nucleobases. In various embodiments, an AON includes at least one 5-methylcytosine nucleobase. In various embodiments, an AON includes two, three, four, five, six, seven, eight, nine, or ten 5-methylcytosine nucleobases.

In various embodiments, an AON includes both modified and unmodified nucleobases. For example, an AON may include both cytosines and 5-methyl cytosines. In some embodiments, an AON may include one, two three, four, five, six, seven, eight, nine, or ten cytosines and further include one, two, three, four, five, six seven, eight, nine, or ten 5-methylcytosines.

In various embodiments, each of a particular type of nucleobase in the AON is replaced with a corresponding modified nucleobase. For example, every guanine of the AON is replaced with a 5-methyl guanine. As another example, every cytosine of the AON is replaced with a 5-methylcytosine.

In some embodiments, an AON includes modified nucleobases that are designed according to the gapmer design of the gapmer AON. In various embodiments, the linked nucleosides of the 5′ wing region, the linked nucleosides of the 3′ wing region, or the linked nucleosides of the central region comprise one or more modified nucleobases. In some embodiments, the 5′ wing region and/or the 3′ wing region includes one to ten modified nucleobases, such as from two to eight modified nucleobases, from three to six modified nucleobases, or from four to five modified nucleobases. In some embodiments, the 5′ wing region and/or the 3′ wing region includes one, two, three, four, five, six, seven, eight, nine, or ten modified nucleobases. In some embodiments, the central region includes one to ten modified nucleobases, such as from two to eight modified nucleobases, from three to six modified nucleobases, or from four to five modified nucleobases. In some embodiments, the central region includes one, two, three, four, five, six, seven, eight, nine, or ten modified nucleobases. Examples of modified nucleobases include a 5-methylpyrimidine, for example, pseudouridine, 5-methylcytosine or 5-methoxyuridine, a 5-methylpurine, for example, 5-methylguanine.

In various embodiments, at least one cytosine in the 5′ wing region and/or the 3′ wing region of the AON is replaced with a modified nucleobase, such as a 5-methylcytosine. In various embodiments, at least one cytosine in the 5′ wing region is replaced with a modified nucleobase, such as a 5-methylcytosine. In various embodiments, at least one cytosine in the 3′ wing region is replaced with a modified nucleobase, such as a 5-methylcytosine. In various embodiments, at least one cytosine in the central region is replaced with a modified nucleobase, such as a 5-methylcytosine. In various embodiments, all cytosines in the 5′ wing region are replaced with modified nucleobases, such as 5-methylcytosines. In various embodiments, all cytosines in the 3′ wing region are replaced with modified nucleobases, such as 5-methylcytosines. In various embodiments, all cytosines in the central region are replaced with modified nucleobases, such as 5-methylcytosines.

In particular embodiments, all cytosines in the 5′ wing region, all cytosines in the 3′ wing region, and all cytosines in the central region are replaced with modified nucleobases, such as 5-methylcytosines. In particular embodiments, all cytosines in the 5′ wing region, all cytosines in the 3′ wing region are replaced with modified nucleobases, such as 5-methylcytosines; however, all cytosines in the central region are unmodified nucleobases.

### Modified Oligonucleotides

Described herein are additional embodiments of modified oligonucleotides, which can include any of the modified internucleoside linkages and/or modified nucleosides (e.g., modified sugar moieties, and/or modified nucleobases) described above.

In some embodiments, an AON, or a pharmaceutically acceptable salt thereof, comprises the sequence of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C), where at least one nucleoside of the sequence is substituted with a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a bicylic nucleic acid, a bridged nucleic acid, a locked nucleic acid (LNA), a constrained ethyl (cET) nucleic acid, a tricyclo-DNA (tcDNA), a 2′-0,4′-C-ethylene linked nucleic acid (ENA), or a peptide nucleic acid (PNA). In particular embodiments, at least one internucleoside linkage of the AON is a phosphorothioate linkage. In some embodiments, all internucleoside linkages of the AON are phosphorothioate linkages. Also described herein are pharmaceutical compositions that include any of the foregoing antisense oligonucleotides, or a pharmaceutically acceptable salt thereof, and a pharmaceutically acceptable excipient.

AONs described herein, can include chemically modified nucleosides, including modified ribonucleosides and/or modified deoxyribonucleosides. Chemically modified nucleosides include 2′-substituted nucleosides in which the 2′ position of the sugar ring includes a moiety other than —H or —OH (for example, —F or an O-alkyl group). For example, chemically modified nucleosides include, but are not limited to 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, 2′-O-(2-methoxyethyl)thymidine, and 2′-O-(2-methoxyethyl)-5-methylcytosine.

In some embodiments, AONs can include chemically modified nucleosides, for example, 2′ O-methyl ribonucleosides, for example, 2′ O-methyl cytidine, 2′ O-methyl guanosine, 2′ O-methyl uridine, and/or 2′ O-methyl adenosine. AONs described herein, can also include one or more chemically modified bases, including a 5-methyl pyrimidine, for example, 5-methylcytosine, and/or a 5-methyl purine, for example, 5-methyl guanine. AONs described herein, can also include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

It is contemplated that in some embodiments, a disclosed AON may optionally have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

In certain embodiments, the disclosure provides mixed modalities of AONs with combinations of modified nucleosides, e.g., a combination of a peptide nucleic acid (PNA) and a locked nucleic acid (LNA). Chemically modified nucleosides also include, but are not limited to, locked nucleic acids (LNAs), 2′-O-methyl, 2′-fluoro, and 2′-fluoro-β-D-arabinonucleotide (FANA) modifications. Chemically modified nucleosides that can be included in AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, an AON, or a pharmaceutically acceptable salt thereof, is a modified oligonucleotide which includes the sequence of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C), and at least one nucleoside linkage of the nucleotide sequence is a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In particular embodiments, at least one internucleoside linkage of the nucleotide sequence is a phosphorothioate linkage. In some embodiments of AONs described herein, one, two, three, or more internucleoside linkages of the nucleotide sequence is a phosphorothioate linkage. In various embodiments of AONs described herein, all internucleoside linkages of the nucleotide sequence are phosphorothioate linkages. Thus, in some embodiments, all of the nucleotide linkages of an AON sequence of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, are phosphorothioate linkages.

Contemplated AONs may optionally include at least one modified sugar. For example, the sugar moiety of at least one nucleotide constituting the oligonucleotide is a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. In various embodiments, the internucleoside linkages of the 5-10-5 gapmer AON can have the sequence of sssssooooooooosssss (where “s” refers to a phosphorothioate bond and “o” refers to a phosphodiester bond) where all the phosphorothioate bonds are in the 5′ wing region or the 3′ wing region and all the phosphodiester bonds are in the central region of the AON. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. The internucleoside linkages of the AON can have the sequence of ooooosssssssssooooo (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. The internucleoside linkages of the AON can have the sequence of sssssssssssssssssss (where “s” refers to a phosphorothioate bond) where all internucleoside linkages of the AON are phosphorothioate bonds. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eee-d8-eee (where “e” denotes a 2′-MOE modified nucleoside and where “d8” denotes a contiguous 8 DNA nucleobase sequence). In this embodiment, the 5′ wing region includes three 2′-MOE modified nucleosides, the gap region includes 8 oligonucleotide units comprising at least 4 contiguous nucleobases, and the 3′ wing region includes three 2′-MOE modified nucleosides. The internucleoside linkages of the AON can have the sequence of sssooooooosss (where “s” refers to a phosphorothioate bond and “o” refers to a phosphodiester bond) where all the phosphorothioate bonds are in the 5′ wing region or the 3′ wing region and all the phosphodiester bonds are in the central region of the AON. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eee-d8-eee (where “e” denotes a 2′-MOE modified nucleoside and where “d8” denotes a contiguous 8 DNA nucleobase sequence). In this embodiment, the 5′ wing region includes three 2′-MOE modified nucleosides, the gap region includes 8 oligonucleotide units comprising 4 contiguous DNA nucleobases, and the 3′ wing region includes three 2′-MOE modified nucleosides. The internucleoside linkages of the AON can have the sequence of ooosssssssooo (where “s” refers to a phosphorothioate bond and “o” refers to a phosphodiester bond) where all the phosphodiester bonds are in the 5′ wing region or the 3′ wing region and all the phosphorothioate bonds are in the central region of the AON. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eee-d8-eee (where “e” denotes a 2′-MOE modified nucleoside and where “d8” denotes a contiguous 8 DNA nucleobase sequence). In this embodiment, the 5′ wing region includes three 2′-MOE modified nucleosides, the gap region includes 8 oligonucleotide units comprising 4 contiguous DNA nucleobases, and the 3′ wing region includes three 2′-MOE modified nucleosides. The internucleoside linkages of the AON can have the sequence of sssssssssssss (where “s” refers to a phosphorothioate bond) where all internucleoside linkages of the AON are phosphorothioate bonds. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eee-d10-eee (where “e” denotes a 2′-MOE modified nucleoside and where “d10” denotes a contiguous 10 DNA nucleobase sequence). In this embodiment, the 5′ wing region includes three 2′-MOE modified nucleosides, the gap region includes 10 contiguous DNA nucleobases, and the 3′ wing region includes three 2′-MOE modified nucleosides. The internucleoside linkages of the AON can have the sequence of sssooooooooosss (where “s” refers to a phosphorothioate bond and “o” refers to a phosphodiester bond) where all the phosphorothioate bonds are in the 5′ wing region or the 3′ wing region and all the phosphodiester bonds are in the central region of the AON. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eee-d10-eee (where “e” denotes a 2′-MOE modified nucleoside and where “d10” denotes a contiguous 10 DNA nucleobase sequence). In this embodiment, the 5′ wing region includes three 2′-MOE modified nucleosides, the gap region includes 10 contiguous DNA nucleobases, and the 3′ wing region includes three 2′-MOE modified nucleosides. The internucleoside linkages of the AON can have the sequence of ooosssssssssooo (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine).

In particular embodiments, an AON has a structure of eee-d10-eee (where “e” denotes a 2′-MOE modified nucleoside and where “d10” denotes a contiguous 10 DNA nucleobase sequence). In this embodiment, the 5′ wing region includes three 2′-MOE modified nucleosides, the gap region includes 10 contiguous DNA nucleobases, and the 3′ wing region includes three 2′-MOE modified nucleosides. The internucleoside linkages of the AON can have the sequence of sssssssssssssss (where “s” refers to a phosphorothioate bond) where all internucleoside linkages of the AON are phosphorothioate bonds. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeee-d10-eeee (where “e” denotes a 2′-MOE modified nucleoside and where “d10” denotes a contiguous 10 DNA nucleobase sequence). In this embodiment, the 5′ wing region includes four 2′-MOE modified nucleosides, the gap region includes 10 contiguous DNA nucleobases, and the 3′ wing region includes four 2′-MOE modified nucleosides. The internucleoside linkages of the AON can have the sequence of ssssooooooooossss (where “s” refers to a phosphorothioate bond and “o” refers to a phosphodiester bond) where all the phosphorothioate bonds are in the 5′ wing region or the 3′ wing region and all the phosphodiester bonds are in the central region of the AON. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeee-d10-eeee (where “e” denotes a 2′-MOE modified nucleoside and where “d10” denotes a contiguous 10 DNA nucleobase sequence). In this embodiment, the 5′ wing region includes four 2′-MOE modified nucleosides, the gap region includes 10 contiguous DNA nucleobases, and the 3′ wing region includes four 2′-MOE modified nucleosides. The internucleoside linkages of the AON can have the sequence of oooosssssssssoooo (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeee-d10-eeee (where “e” denotes a 2′-MOE modified nucleoside and where “d10” denotes a contiguous 10 DNA nucleobase sequence). In this embodiment, the 5′ wing region includes four 2′-MOE modified nucleosides, the gap region includes 10 contiguous DNA nucleobases, and the 3′ wing region includes four 2′-MOE modified nucleosides. The internucleoside linkages of the AON can have the sequence of sssssssssssssssss (where “s” refers to a phosphorothioate bond) where all internucleoside linkages of the AON are phosphorothioate bonds. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeee-d8-eeee (where “e” denotes a 2′-MOE modified nucleoside and where “d8” denotes a contiguous 8 DNA nucleobase sequence). In this embodiment, the 5′ wing region includes four 2′-MOE modified nucleosides, the gap region includes 8 oligonucleotide units comprising 4 contiguous DNA nucleobases, and the 3′ wing region includes four 2′-MOE modified nucleosides. The internucleoside linkages of the AON can have the sequence of ssssooooooossss (where “s” refers to a phosphorothioate bond and “o” refers to a phosphodiester bond) where all the phosphorothioate bonds are in the 5′ wing region or the 3′ wing region and all the phosphodiester bonds are in the central region of the AON. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeee-d8-eeee (where “e” denotes a 2′-MOE modified nucleoside and where “d8” denotes a contiguous 8 DNA nucleobase sequence). In this embodiment, the 5′ wing region includes four 2′-MOE modified nucleosides, the gap region includes 8 oligonucleotide units comprising 4 contiguous DNA nucleobases, and the 3′ wing region includes four 2′-MOE modified nucleosides. The internucleoside linkages of the AON can have the sequence of oooosssssssoooo (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeee-d8-eeee (where “e” denotes a 2′-MOE modified nucleoside and where “d8” denotes a contiguous 8 DNA nucleobase sequence). In this embodiment, the 5′ wing region includes four 2′-MOE modified nucleosides, the gap region includes 8 oligonucleotide units comprising 4 contiguous DNA nucleobases, and the 3′ wing region includes four 2′-MOE modified nucleosides. The internucleoside linkages of the AON can have the sequence of sssssssssssssss (where “s” refers to a phosphorothioate bond) where all internucleoside linkages of the AON are phosphorothioate bonds. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine).

In particular embodiments, an AON has a structure of eeeeee-d11-eeeeee (where “e” denotes a 2′-MOE modified nucleoside and where “d11” denotes a contiguous 11 DNA nucleobase sequence). In this embodiment, the 5′ wing region includes six 2′-MOE modified nucleosides, the gap region includes 11 contiguous DNA nucleobases, and the 3′ wing region includes six 2′-MOE modified nucleosides. In various embodiments, the internucleoside linkages of the 6-11-6 gapmer AON can have the sequence of ssssssssssssssssssssss (where “s” refers to a phosphorothioate bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. In various embodiments, the internucleoside linkages of the 5-10-5 gapmer AON can have the sequence of sososssssssssssosos (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. In various embodiments, the internucleoside linkages of the 5-10-5 gapmer AON can have the sequence of soooossssssssssooos (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. In various embodiments, the internucleoside linkages of the 5-10-5 gapmer AON can have the sequence of soooossssssssssooss (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d8-eeeee (where “e” denotes a 2′-MOE modified nucleoside and where “d8” denotes a contiguous 8 DNA nucleobase sequence). In this embodiment, the 5′ wing region includes five 2′-MOE modified nucleosides, the gap region includes 8 oligonucleotide units comprising 4 contiguous DNA nucleobases, and the 3′ wing region includes five 2′-MOE modified nucleosides. In various embodiments, the internucleoside linkages of the 5-8-5 gapmer AON can have the sequence of sossssssssssssoss (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeee-d10-eeeee (where “e” denotes a 2′-MOE modified nucleoside and where “d10” denotes a contiguous 10 DNA nucleobase sequence). In this embodiment, the 5′ wing region includes five 2′-MOE modified nucleosides, the gap region includes 10 contiguous DNA nucleobases, and the 3′ wing region includes five 2′-MOE modified nucleosides. In various embodiments, the internucleoside linkages of the 5-10-5 gapmer AON can have the sequence of sosssssssssssssosss (where “s” refers to a phosphorothioate bond and “o” refers to a phosphodiester bond). In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

In particular embodiments, an AON has a structure of eeeeee-d10-eeee (where “e” denotes a 2′-MOE modified nucleoside and where “d10” denotes a contiguous 10 DNA nucleobase sequence). In this embodiment, the 5′ wing region includes six 2′-MOE modified nucleosides, the gap region includes 10 contiguous DNA nucleobases, and the 3′ wing region includes four 2′-MOE modified nucleosides. The internucleoside linkages of the AON can have the sequence of sssssssssssssss (where “s” refers to a phosphorothioate bond) where all internucleoside linkages of the AON are phosphorothioate bonds. In various embodiments, the AON includes unmodified cytosines. In various embodiments, the AON includes modified cytosines (e.g., 5-methylcytosine). In various embodiments, all cytosines of the 5′ wing region and the 3′ wing region are modified cytosines (e.g., 5-methylcytosine). In various embodiments, all cytosines of the central region are modified cytosines (e.g., 5-methylcytosine).

### Target Gene Product

Generally, an AON disclosed herein includes linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a target gene product. Example target gene products include any of PPM1A, ATXN2, SOD1, or MAPT gene products. In particular embodiments, an AON disclosed herein includes linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a gene product. In particular embodiments, an AON disclosed herein includes linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of an ATXN2 gene product. In particular embodiments, an AON disclosed herein includes linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a SOD1 gene product. In particular embodiments, an AON disclosed herein includes linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a MAPT gene product.

In embodiments of the invention described herein, an AON can target gene products of genes of one or more species. For example, an AON can target a gene product of a mammalian gene, for example, a human (i.e., Homo sapiens) gene, a rodent gene (for example, a mouse (Mus musculus) gene), and/or a primate gene (for example, a Macaca fascicularis gene or a Macaca mulatta gene). In particular embodiments, the AON targets a human gene product.

A gene product can be, for example, an RNA gene product, for example, an mRNA gene product, or a protein product of a target gene. In some embodiments, the AON includes a sequence that is at least 90%, at least 91%, at least 92%, at least 93%, at least 94%, at least 95%, at least 96%, at least 97%, at least 98%, at least 99%, or that is 100% complementary to a sequence of a gene or a RNA, for example a mRNA or a pre-mRNA, or a portion thereof. In some embodiments the AON includes a nucleobase sequence that is complementary to a portion of a sequence that is shared between genes or RNAs (for example, mRNAs) of multiple species. For example, in some embodiments, the AON is an antisense therapeutic, for example, an AON that is complementary to a sequence shared by a human, mouse, and/or primate genes or mRNAs.

### PPM1A

In some embodiments of the disclosure, the PPM1A gene product is a PPM1A mRNA or PPM1A pre-mRNA comprising sequences from nucleotide 41,932 to nucleotide 42,787 and from nucleotide 44,874 to nucleotide 44,990 of a PPM1A gene sequence (for example the PPM1A gene sequence of NCBI Reference Sequence NG_029698.1 (SEQ ID NO: 1909) or a PPM1A coding sequence), or a portion thereof. In some embodiments of the disclosure, the PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA or PPM1A pre-mRNA comprising sequences from nucleotide 41,932 to nucleotide 42,787 and from nucleotide 44,874 to nucleotide 44,990 of a PPM1A gene sequence (for example the PPM1A gene sequence of NCBI Reference Sequence NG_029698.1 (SEQ ID NO: 1909) or a PPM1A coding sequence), or a portion thereof.

| | |
|---|---|
| 1 | ttatttttct gaatgccttc aaatgtgaca agtatgctct actgatcccc attgtgtcag |
| 61 | acccagcctg atctaacatt tatattacca gcctggtccc agtcaaagga gctggagttt |
| 121 | tcagattctg gtgctaacct gaattctacc taccacttct ttacaaagag gcctctgccg |
| 181 | tgtttgtacc agcggcagga tgacttgaag ctctccatgg acatgcgg g ggggtggagg |
| 241 | tgttgatgga tattgtcttg gcttttgcct tatgtgggat ttttaagaaa tgtgcaggaa |
| 301 | aaaaaaaaca ggaaagtgta ttaggagaag gcaggataga agttttcctc acgtggagag |
| 361 | agcaggagaa tggaggaagt gactgctgga agcaaaactc taagaaaaaa ccaattttac |
| 421 | ttggagctta aaaaagattt tgctgtgtaa tgtgcattgt aactcttctc ccatctcccc |
| 481 | aaggcatctt tggtaaagat gtcattgttg ggctttttgt tttccatggc acaatgcact |
| 541 | gaaagggcct caaggggaaa gcaagtcatc tctggaaagc ttagccagaa tttgcaacag |
| 601 | aaagagtggg aattttggaa tcaggtccta ggcttgcaaa agagctctct ttcacagcct |
| 661 | tagcttcctc ctttgtaaaa tggaaaaaaa aatagtttct tttttgcaaa gttgttgtga |
| 721 | gaattaaata acactcatga ggggcattgt atggagcttg gcacattcza ggccctcttt |
| 781 | ttatattgaa ctaatatgat cttctctgta attcccatga attaattcca gttctatttt |
| 841 | gcaaaacatt gattctttaa cctttttcat gtaatggtgc tccagtttgt ctagatcctt |
| 901 | caagaagaga aaatgcatat caacatcttt gtatctagtt gctctcaagc ttaaaatgat |
| 961 | gtcatttttc aattattgct tttattatac agtaaaaaca tatttagaag agactcaaga |
| 1021 | taaatattaa aagttaagct agttactgaa caaatttctt aaatatttac gaaaaataag |
| 1081 | atcaacacac atttgaccat ggcagctttt ctaaaggcaa aggatctaat ttggtgatta |
| 1141 | actaaccata ttgagcactc attaggtgtc agggatgtac cagttcttct actgtatttg |
| 1201 | ggtgttttta attatgaata accaaataca aaaatcaaag tagtttaaac aatgaggagc |
| 1261 | atttattatc actcttaaaa ataaattccc aaggtgggca attgcacgga tggctcaatg |
| 1321 | gctttacaat gtgaaagatc cagccacttg ctatcaattc ttttctctac attgccttgg |
| 1381 | ttctttgcaa gataaccttg aaattccagg ctcacatata gaaataacaa catatggctg |
| 1441 | aggaaggcat tttctctcct gtgattcttc ttattaggga agaaaacctc tctaccccct |
| 1501 | caagacttct caatttcctt tatccagaat tgggctacct cactatgtcc ccaggcagtg |
| 1561 | cttctcaatt ttttcacacc atgacacaaa tacaaagata acgtttttgt gacacagagg |
| 1621 | ggtacctggg taatgttgct tgtggccagt cataccatcc cagggcttca gcagctcagc |
| 1681 | ctcacttgaa tgtcccaagg gcttaaggga tccatacctt atcacatcta tgacccatct |
| 1741 | gagacatacc agggtatgct ggcacataga ttggtatgct ctgctctaag ctacaaggga |
| 1801 | gggtgagaga gcatgcatcc atatacatgc tctagtgaga gaaggatctg ccaacgagga |
| 1861 | ggctctgcca agtagtagga agtggtgggg caaccaaccg catcagtcat cttttcaagg |
| 1921 | gtgccttcat ctattctaat tttcacaaca cattttagga ggaagtattc tgtggaggat |
| 1981 | aaaaccaagg cttataattt aaatgacttg cctgaggtca cagaaccagt gagtggttca |
| 2041 | attgacacag cagatgcaga tccaggtgtg tgctgggaaa tgtttaacaa ccacctttcc |
| 2101 | aaatgtagtt ccaacatggc tattggttaa tactttcatt taagataaaa ataaaacaac |
| 2161 | aaaaatgtat gttagagctt attagtcaat gatgtgactt cattgctaaa ttgaatgttt |
| 2221 | ttatgctatt caccatgtaa cagttataga cacgacatgt cttttacatt taatctgcat |
| 2281 | aaaacatttt ctctgtcact taagcctaga ccaagcttgt ccaaccctgg gcctgtgggc |
| 2341 | cgcatacagc ccaggatggc tttgaatgca gcccaacaca aattcgtaaa ctttcttaaa |
| 2401 | atgtgagatt ttgtgtgtgt gtgtgatttt gtttcgtttt gttttgtatt agctcatcag |
| 2461 | ctatcattag tgttagcgta ttttatgtgt ggcccaagac tatttttctt ctgccaatgt |
| 2521 | ggccgaggga agcaaaagat tggacaaccc tggtctagcc cacaaccagc aaagcaataa |
| 2581 | gtgaagccct catctgtggt gtccgctgat caagctacca aagaaaactc actaaatgtg |
| 2641 | gtattgggaa gagatgtaca attacatata gggaatttcc accatgcaga cacaatgcat |
| 2701 | gtaaattgcc taaagaacaa agacaatatt aaaatgtatt aacataattt ggaagtgatg |
| 2761 | tttttatcac atactacctt tgtttaaaat ttatttcatt ggaagtttat ctaacttaat |
| 2821 | ttttatttct atttttatat ttatttattg attgattttt gagatggagt ctcaccttgt |
| 2881 | cacccaggct ggagtgcaat ggcgcaatct cagctcactg caacctctgc ctcctgggtt |
| 2941 | caagcaattc tcctgcctca gcctcccaag tagctaggat tacaggctcc cgacaccaca |
| 3001 | cctggctaat ttttgtattt ttagtagaga cggggtttca ccatgttggc caggctgatc |
| 3061 | tcaaactcct gatctcaggt gatccaccca cctcagcctc ccaaagtgct gggaatatag |
| 3121 | gcatgagaca ctgcacccag cctatatcat ttaattttta ataatgagta tttaacaaac |
| 3181 | agcaggcaaa atttctgaaa atttaataat ctgctgtcat gagccagtat aggccagctc |
| 3241 | tccacaccac ggactggaac taagttcagc actctttcca ctcctgctgc ctttactacc |
| 3301 | agcatgtaaa agcaatgatg ctcagtatcg tacgggggac aaacctgggt acccgtcatt |
| 3361 | aatttctgtg accctgggta gttactacca gatgcttagt tttctcttct ataaaggagg |
| 3421 | agattaagag tccttgccct gtctacctgc aagggttttt gagaggatta aactggttag |
| 3481 | tctgtatgat agtgctttgt aaactgcaga gtcaggttca gataaaaggc tttatgatga |
| 3541 | tgacaatttc attactgttt tcaaccaggc caccattgtt aaggatgcag tgtgtcgctg |
| 3601 | ctttcaaaag tatggtttag tttgagatct tctgtaatgc ccaggataga cagtaaacac |
| 3661 | ccagaagaat tgttctgaag aagcccactc aaatctgtgc tagtctttac ttagcacaaa |
| 3721 | taggtaccta tattgggatg aaaactattt atttgaagct tataattcac tcttttctag |
| 3781 | ctgctgctgg attgtctgac gggtctttcc tggagttatg gaatacttct caatttcaat |
| 3841 | agttgaataa aggtgttttt ttttctgtca tgcaggctag ggtgcagggg catgatctcg |
| 3901 | gcccactgca acctccacct cctgagttcc agtgattctc ctgcctgcga ttctcctgcc |
| 3961 | tcagcttccc aagtagctgg gattacaggc gtgtgccacc aggtgtggct aattttgtat |
| 4021 | tttcagtaga gacggggttt ccccatgttg gccaggctgg tcttgaactc ctggcctcaa |
| 4081 | gtgatccact cgctttggcc ttccaaagtg ctgggattac aggtgtgagc cactgcaccc |
| 4141 | agactatttt taatttcaat ttttaaaaaa gttgctttgc tttttgcacc agaccaactc |
| 4201 | accaagttat tttctatgga ttatacccgt gcacataatg tagtttcctg attataaatt |
| 4261 | aaatatccct aatgtccacg ttgaaacact ctcgaaactt aaatgaccat aaactatttg |
| 4321 | ccttgagttt gctgaacttg tctttgggct gtaagaaact cttttctgcc aagctctttt |
| 4381 | ccacatcagg ccctttgccc atgctgaccc ctctacctgg aaagttctcc cctgattatg |
| 4441 | aagctgactc cttctcatcc tttagatcaa atgctgcctt cttagagagg tctgcactga |
| 4501 | acacctgttt caagtcgtct cccggatctg tttccttatt tattttcttc acagcatgga |
| 4561 | taacagtatg caatttcctt tctatctgct gacttatttg tctatgcctc ttattattat |
| 4621 | gttggctctt tgtgggcagg gcttgcccat tgatgtcatt atctacctcc agtgtcccac |
| 4681 | acagtgcttg gtgcacaata gctatctcat gaacagatga acttatctga caagatccac |
| 4741 | tctagtattg taaggctgtg gccatatatg gctatcttga ggtgaaagcc taccctaaag |
| 4801 | actgtccagg acttggcctc tggtgctcac agttgcccag gtttctctca ggtcgcagga |
| 4861 | tgagccacag tggtagcata agactgctgg ctgctcccct ctgcccctcc ctaatggggc |
| 4921 | tgcccagtga tgtcacacag gacaggattc tctaatcctt ttgattctgt aacagtcctc |
| 4981 | taagcagcta agtttctcat aatgtattat gatgtagggg agggaggaag gagggccagg |
| 5041 | gtagggggca caccctctcc ctgaatgaaa ttcatttagg ggcactgtct gctcgcatgt |
| 5101 | tctgttctgg gagaaaatgg gtagcagaag ctacaatttg tactaagcta atgaaaagag |
| 5161 | agaaaagaag aatggggaag agaagggcaa agaaggcaaa aagagaagag aagaaaaagg |
| 5221 | gaggagagag aaggagaaat gagaaaagag gaaaccaaat gaagaggatg tgtgagagaa |
| 5281 | aaaaatatga aacaggtagt gagggaggaa ggaattgttg aacctatgtt ctggttggct |
| 5341 | ttctaactct tgagtagtga ctggcttcag tggaaaagga tggtgagggg tattctcaaa |
| 5401 | tactagcctc ctgagggggt catttacatt actgcatata ttttgagagg cccaggcaag |
| 5461 | tctgcctcaa gggtgactct cctggagagt aatttgggag caggaatgta aacctttttt |
| 5521 | taagtcacaa ttgtggtgga aagggaaaga gagtattgct gatcagatta agctttagac |
| 5581 | tccacactac agcccctgtc ctcaccccaa gcctcccaat tactaggcta tcaacacaca |
| 5641 | ctaaattcag gactttaaag ccaatcttgt agataatctt tttctttgct aattgaaata |
| 5701 | taaacagatt tccaaagcag agcaggatag ttttataata tacctataaa aattactaaa |
| 5761 | atgtcatcat catggcaata tattcccact aaaatcttac tgtgaacatc ccaaatatgt |
| 5821 | gtagaggccc ggcaaagtct tacagttcag aagagaactt tcaatcctct tttttcaatg |
| 5881 | ttattttccc cttctatctg tccccccttc ttagcataag gtttaataaa aggggatatg |
| 5941 | atgatattaa acatatttaa tcagacaata ttgatatctt tttaaaatat taatcttata |
| 6001 | acccatgagt ttcctagttc ataccaaaat gtccctgcaa gctgtattca gaaataaata |
| 6061 | gtaggaaggt gctgctttat cggcagtctg tacaatgcgt agactcctta aaaatttctt |
| 6121 | tctgttcata ttttctattt ctctccctct tcttctacaa ttgacccaga ttctccaagt |
| 6181 | gcagaacaga gagaagaaag gtgtggaagc ctaaaggaag gaagcagaaa gataggcagg |
| 6241 | catttcaagt tttctatggt ccactaccgt cactagtgac ccagcttctg cttataaata |
| 6301 | agaaaatttg tcttttaaga gcactctaaa tggttgccat atgagctttt gtagttcaga |
| 6361 | atgttaatga gtccaccagc atgctactca tgctgttact tgcctggagt attaaggatt |
| 6421 | ccaagaaggt agcgtttcaa agatctctga tctaatttct ttttttgggg ggtcggggag |
| 6481 | atatgtggag aattaggaaa tgtatcctca gatgatactg agaccaaatg catagtgaat |
| 6541 | tttattttta ttttgtatat gtggaaatga tccacgatca tcttctaagg aattctggcc |
| 6601 | gggaacagtg gctcacgact gtaatcccag cactttggga ggccgaggag ggtggatcac |
| 6661 | gaggtcagga aaccgagacc atcctggcca acacggtgaa accctgtctg aactaaaaat |
| 6721 | acaaaaaaat tagccaggcg tggtggcagg cgcctgtagt cccagctact cgggaggctg |
| 6781 | aggcaggaga atggcatgaa cccgggaggc ggagcttgca gtgagccgag attgtgccac |
| 6841 | tgtactagcc tggatgaaag agcgagactc tgcctcaaaa aaaaaaaaaa aaaaaaaaaa |
| 6901 | aaaagtgaag gaaacagaac cttccccaaa gtcatgttga gaaaattaga gatggctttt |
| 6961 | tacaacttta aaagcagcag ctaaatattt gctgaatgcc agaaagaata aataggttgc |
| 7021 | ttttgatcta agagttgggg caacaaaatg gtttaggaga tagaaagtag tagggtgcaa |
| 7081 | attgctatag tgagctcctc aagtaaaata gagaggcctt caacataaat ttaccttttt |
| 7141 | tactggccag aactcaattt ggccagtaaa agaacctgcc ttctacaatg aagtctttgg |
| 7201 | tttgtgtgtg catcagaaca tcttttaaaa atacggactt ttcttctaac ttcttttcct |
| 7261 | gcgaatgtgg tgtaggtcaa attcgtgaac tggttaagcc gctgacaaga gagggaaggg |
| 7321 | caccccagcc aatgggaacc cgtgctggtg atgtactgct cctaggctgc gattcattgg |
| 7381 | ctgcctaggg tttgtcattc tatccccggc gtctgcaagc agcgtgggtc gggttttcca |
| 7441 | gctagctaaa ctggattttg gccgctggca cgtgcagttt ctattaactt tagctcatct |
| 7501 | ctacctctta atagaagcag ctgttatgtt tctgattatt ctcctgattt tgaaaaatcc |
| 7561 | tgttccctta acctacctca ttggaggtcg ttggaggtcc cctgggacag aaccacgtga |
| 7621 | ttatctaaca tgtgcaaata aatacaatgt ggaatcccct gaactataga gggaaaacct |
| 7681 | gccagaaact cagagcatgt gccccgtgcg tgaatcagtt gcactcgcac attggtgggc |
| 7741 | ggtggtagcg tgcttgtgtc tctgcccttt gggtcgcgag gtgtgtggcc gtggcacctg |
| 7801 | cttgggcttc ctggggtcac tgggagagga aggctctgga ggagtgggac tctggggtag |
| 7861 | gggggtctgg cagctgcctg ggacccacca tgggttcgaa gggcagcggc cagaccgcga |
| 7921 | cggcgcccaa gaagggtgga cagcttcggg tctgtgctcg gggctgcgtg tacaccatct |
| 7981 | tcgcagggag tggggtttgt atctgcaggc aggtagaaga gccagggctc cgggcgcgga |
| 8041 | cgccgtaggg gtagcttccg agtccctact tggccaaggg ccggcacgga ggggctgtcg |
| 8101 | atctactggg cacctaatag ccgcacattc tgtccagtcc ctggcgccgg aacgtgaagg |
| 8161 | cccgggtcct ggctgctatc cgggatccgg acgctccctt cggcttcggc ctggtgctcc |
| 8221 | aggccgtaag gcaaaggcgg cggcagcagc agcggcagag aaagaagctg ggtaaggcag |
| 8281 | tgacgtcatc tgtggcggcc cctcccccgc ccgccagccc tcaccgcagt tcctgcccaa |
| 8341 | cgacatccgg gtctctgtag ctgcgcgccg cgccgcagct gtagcggcgg cggagccagc |
| 8401 | ggggcggggc gagcggaggg gtgggggagg ggcgacgcgg tcgtgagcgc gcctgcgcgg |
| 8461 | ggccgcgcta gaggcggcgg cggcggcggt ggcggcgcta gggacgggag cgcgcgcggg |
| 8521 | agctagagag cagtggtctc ggcgctcgtc cggcccgcag cttcgggtcc tcaggcggct |
| 8581 | gttgctccgg aacgggtggt tggggagggg ggggtggggg gactctagac agctgaggcg |
| 8641 | cgaaagcgat gagtcctcgg ctcttcctcc tccttctccg ggacccgctc tctgcctccc |
| 8701 | tctccaacgc ccggatgatc tgagccgcga gggcgccgac agccgggggc ccggacgcag |
| 8761 | cccggctcct cccctcctcd gccccttccc cagcctgacc tggcccgccg ctgcagcggt |
| 8821 | gacccctccc ccggctgccg ccgtcgccgc cgcggtgacc ccctccccgg ctgccgccgc |
| 8881 | cgccgcctcg gccgaccagg gacctgcccg cctgcggctg ctccgggtaa gtgcggcgct |
| 8941 | cgggccgacg gcgggctggc gggcggtgcg ggcctgcgcg gcggcggcgg cgggcaggcc |
| 9001 | tggggcctgt aaacaagccg ggcgtctgcc cgggcgctcc cgggaggaga cgcgacaact |
| 9061 | ccaccccctg gccggcctcc tcccccgagc cgggcggcgg acggcgaggg gttaacgctc |
| 9121 | ggcgagggcg gtggcgggga ggcgggggcg gggtgttagt tgcggtggcg cggggagggg |
| 9181 | gcgcgaccct ctggccgtcc gcggccgaga tgggtgtttg tggcggcgaa gcaggcgacg |
| 9241 | gccgcaggcc ggcctggccg gagagcggcc tgcagaactt ttcccgagcg gggagggggc |
| 9301 | gtccccgcgc ccccacccgc tcgggcgcgc cgccccggct gtcctgggct tcagcccctg |
| 9361 | ggagggaggc ggggagactt cctttgaacc gggcttagcc tcgaaaaacc taattctgta |
| 9421 | gaaattgctt aaggttttac agggggcagg gctgagagac tccttcagaa ctcaagtcag |
| 9481 | tgagtgatgc ctctagttca ttcattatta aggcatcgaa caaatccatt gaagtaaatt |
| 9541 | ccgatttctt atccttttaa ctacgtaaat cgggtaaaac ttcttgatgt tagagcttgg |
| 9601 | ttgaggtatg gttctgttct gcttgtagat tttaaataaa cttggagcct tttcttgaaa |
| 9661 | gacagctggc tgtggagagc cttctaccaa ctgcagtttt agatacgttt tgaaaatttc |
| 9721 | atcacctgag gtatttttct tctctcctga aacaccacat ggtacaaatc cacaaaagtc |
| 9781 | atgcttcagt tgtccaagcc atgggatctg tgtgctagcc ccacctcccc gcaagtttcc |
| 9841 | tgctcattgg aataatgagc tgaatgtgtt ttaagaagtg gttggaagag tatgagagat |
| 9901 | aggtatcatt ttctcaaagg aaatgatttt gacattattg agagaaatta gacattttaa |
| 9961 | caattgtttt gtaaaacagt cttttcaaac ttgcacgttg gggttttaaa cacaaccact |
| 10021 | ttacatgtgt tgcgctccga aaagtggagg gatttgttga aacactactc tgctgagact |
| 10081 | tagggttcca gagcacttgg gaatttagca tagcttggtc cccaactctc tttgaaacac |
| 10141 | tttactttta tacttttatc ttgctacata ttgggccttt tccaaagatt gtatttctgt |
| 10201 | aactccttat tttacaaatg agaaaattga aaataaggag attctctatt cttagatgct |
| 10261 | gcttttactg acttaatgct ttctttacca ataaataatt tgaagcacaa agtaacattt |
| 10321 | cctagtgtta tagttttgtt cttgttatgt gtaacttata tgtatctgat taaactttca |
| 10381 | gtcatgtaag gatgattact tttaacctgg aagtttacag cagtaccaca ttattgaaac |
| 10441 | tataatgaac ttcctgcaca tggcagttgc ctaagacatt ttaatttgat atcataaaac |
| 10501 | atttttagat cggtgaagta attatattat gggaaaggtg tattaagact gataatgttt |
| 10561 | gaagttaact acctcaatta tatttgagaa ataatagtca tcaaattgct ttttatttgg |
| 10621 | cttgtatctt tttatgtatt ttgtgtacat attgtatctt tttacatgag gggctgaggt |
| 10681 | atatgacctg ttgaagggaa cagtgcagta ggagacaagg attgtttagt catggctgtg |
| 10741 | ctccaaaata tctgtgtcca tggacaagtc ctttaacctt ttgagtttca gcttccaaat |
| 10801 | cagtaaagcc agttgggcag attatattat cacatgtact gtccagttac tacatttaaa |
| 10861 | aatttttttt tatcaatatt gtgtgtctag tcaacatacc gttgccaaat aaatgcctgt |
| 10921 | tatgtgtcag gcacttagat gtttcagtgt tctaaggctg gatttctttc tcggttctgt |
| 10981 | tttatttaac aaattctaag ttggtgatgg ttctttagaa aagctgttct tgtgttcttg |
| 11041 | taagctattg atttttcttt tgggattggg atgtgtaggg aaactcttga gaggaaggag |
| 11101 | gatggagatt tcatgggaga caaagggtgg ttgagactac aggtttgaac aagcacagga |
| 11161 | cccttggtag tctgatgtga aggacattat aaaaacacat tatgaaggtt ttttctgtca |
| 11221 | cttcaagctg ggtcttgtta aacctttgaa aattctctaa tacctcattt gtttttgaaa |
| 11281 | gaaatctata gcaacatcat tttgtcatgt ggtaaatatt gctgagcagt cactaaatac |
| 11341 | ctatagtctt ttagtttgtt cagattacat ctaggccatt tgtttactct aatagctaat |
| 11401 | agaatgtaca gctgattttg gtttaactgt gcgtgcatat ttgtaatttt taaccaagaa |
| 11461 | gtggcatatg tccacaagga acttgttatc tctgaaatac tcacccatga gagctggatt |
| 11521 | aattttttaa gatgtaaatc tatgctcatg tagaaataat ataacaaggt gatattttga |
| 11581 | tgatcctctc aaatgattat ctaactcttg cctgggcacc tgactctgag tcagtatact |
| 11641 | cttgaatact ttccatcctt aatcaaatca ggagaaagag atggccatgt aagaaaattg |
| 11701 | gccacggcag ggttttataa gagttaagca agaagataaa acaacacatg gccctgtgaa |
| 11761 | atatataaac cttggagaag aggagaaggt atagtgaggc aactgggtaa aagttgtgtc |
| 11821 | ttcctctaag taattttatg tcacaatatg tcctgtcttc catctcaaat aacaaatttt |
| 11881 | aaaattaagt tgtgtttgtt attttgctat tagccaagat atataaatta actgacctgg |
| 11941 | gattattagc catttgtata tgaaataaac ttgggttaac ctatatcaaa atcaaaagat |
| 12001 | tgattatatt ctttaccctt cactctttca aatgtcttat gttctccata ttacgtaaca |
| 12061 | attttatact tcattctgag tctccagaat atattctttg atgattttga cctttgaatg |
| 12121 | tcctggctat ccatgtacta cctagataat ctcgttacca ggcttttctt ttgctgaggc |
| 12181 | aggtcagagc tagagaaagt aagcaattga gagaggcaag tttctgtata cctgtatcta |
| 12241 | aaatgtaata gactttataa tttttgtaag gaactgagga aaggtacagg cttttaatgt |
| 12301 | gtatcagtaa actagactct tatacagttt ttataatatt agagatgttt taagcttcct |
| 12361 | tgagtaaata tgtgataaag gggggatctt gagtttttat aattcttaat atgtttattt |
| 12421 | ggaatccatt gaccaagtgt atgtagtatc catatttaag atctggatta aaagtgatta |
| 12481 | gtttctgtgg ctgatacgat ttagaattaa gatcttttga gactaaacta cttctagata |
| 12541 | cagaagaacg tcatgaaagc cgtactttct tggccattta tttgtgttag gctttaattt |
| 12601 | tttgccataa ccctctaaaa tatgtgatgt tatcttcatt ttataaataa ggaaatacag |
| 12661 | ggtcagagaa cctaaataac gtgccaggat cagatctggg tagatataaa agtatttttc |
| 12721 | aagatattac tgagtggatt tttcccaatt agatgaacct ccttattacc aggaaccaaa |
| 12781 | atttcaaaag ctaaacatta acacttacga ttcatagatt cctagagtta agcaagtcca |
| 12841 | tgttagttta tgtaaagtct tccaatgcaa gactaatgtt ttcttctaaa acattcagaa |
| 12901 | aagatttaag aaattaagtt cattttctca ttcccaaagt ttcagaagct aacaaagcta |
| 12961 | aacattttat gtcttcaagt accattgttt aggttaagga atggaatcca tgttaattct |
| 13021 | caagtggttg atatccttaa tgaaggatct agggaaatgt ttctgaacag atgtaatcag |
| 13081 | tagagccaca ttgaagggcc ccgtggtgga catggtgata aaactaacct ttgaaagaaa |
| 13141 | ggacagtttg agtccctgtt ctcctatttc gtagccaggt gttcctgggt aagctacttg |
| 13201 | atttcttaaa cctgtttttt tttatgtcaa gtggaaatac ttttgtcgta aggttatctt |
| 13261 | gaagatttaa tgaaataatt atataaagca tttgagatct tttctaccat gaagaaagtg |
| 13321 | ccaaataaat ggtagctgtt attattgagt atatgaaatc ttgagatact taggggtata |
| 13381 | gagacttgct tagtatgaga gggttgtctt tcagtatagc ttttgcagtg tgcctggaag |
| 13441 | ccaaactaga gattgagtag agaatttaga agacattgta aactctgtga gtttagagag |
| 13501 | cttgcatttg aatagactaa aagttataga agaaagagaa agttgagtat gagtagcttt |
| 13561 | atttggctct gcaaagctgg caagatgtca agggtttgga gttaatgatt aggtgggagt |
| 13621 | agatggttaa attgcggata aagagtaaca gtaataaagc aaaggtgaat gttcaggcta |
| 13681 | gaatgatcag tcagtaagga aggacaaggg ttggtatttt gaagaggagt tacagtttat |
| 13741 | ggttagaaat gagcaggaag atatcattgc tacaggaaag gatgcaaaag agtaaggaaa |
| 13801 | ctaaatcatt ctgattaact cagttattac cattttttaa aaatggagtc tttataaaag |
| 13861 | gcgttaaggc tttatatgtg agttctaggt taagtctgaa aaattgtcct ggaatagtgt |
| 13921 | gggtcttact actgcagacc caaaccaaga tattagcatt ccaactggtc tctttcctgc |
| 13981 | tagcatcttg tactgcatct cctccctgat tctataaacc atcaaaattc tttctaaact |
| 14041 | atggtttttc attatatcat ttctctcagt atacttcagt gactcccatt tacctctgga |
| 14101 | ataaagttca gaattcttgg tttggctttt gacactttcc atgattggac ctcaagacat |
| 14161 | tagtctgtta taaccaaact agtacacttg ctatctctgg aacacgctct tttctctcca |
| 14221 | tggatctttg ctcatgctag tgtgcagtct ggaatttgtg tctttccttc tccagctttg |
| 14281 | gttttctctt ttagcaaagc tcatttctaa tatttcttct tccacaaagt catctctgac |
| 14341 | ccccagagtg tttaatctct cctttctctg tacttaaagc aatgtctgtg tattatttct |
| 14401 | atcatatgtt tttttttttt ttgttttgtt ttgtttttga gacagagtct cgctctgtcg |
| 14461 | cccaggctgg agtgcagtgg cgcgatctcg gctcactgca agctccgcct cccgggttca |
| 14521 | cgccattctc ctgcctcagc ctcccgagta gctgggacta caggcgcccg ctaccacgcc |
| 14581 | cggctaattt tttgtatttt tagtagagac ggggtttcac cgtgttagcc aggatggtct |
| 14641 | cgatctcctg acctcgtgat ccgcccgcct cggcctccca aagtgctggg attacaggcg |
| 14701 | tgagccaccg cgcccggcct atcatatgtt ttacctttag atatttgtgt ctaaattata |
| 14761 | attgcattca gttaattcta aagggaaaaa taaattttta ctttacacaa aggctagcgg |
| 14821 | ctagcacagt gctttatgga tagtagacat tcagatattt ggtaagtatt aagcaaaatc |
| 14881 | tacctggagt gaaagtgatt gttgtaggag gaggtagtag aagtagttac aagctttatc |
| 14941 | gtagcagacg tttctgactc tcttcctgaa tgttattttt tctgaacctg gatatttaag |
| 15001 | tgtgaagatt atacaaatct gaagtgtggc agtgttctta tattgatgta gaagactagg |
| 15061 | ttgactgatc ctggacctac aagcttcatt ttaacttcct acattttggc tttgagggtt |
| 15121 | aacaggacat ttataatcac atatctcttt tcagagtatt ttacatttgc aaatattatc |
| 15181 | atgaactttg tgtcctttta accagagagg aattgtggaa tcacaggaat tagatgatgt |
| 15241 | gttaaagaag agagataact atcagctgat ttaatttgaa cccaacaagt atctaataat |
| 15301 | atgcatactc tgtgttcaaa taaacctcta agctagactt tcaagcttat caaaatgcta |
| 15361 | gactattttc agtgtttaga aaataccagc caggtgtggt ggcttatgcc tgtaatccca |
| 15421 | gcactttggg aggctgaggc aggtgaatca caaggtcagg agatggagac catcctggcc |
| 15481 | aacatggtga aactttgtct ctactaaaat acaaaaaatt agccaggcgt ggtggcacgg |
| 15541 | gcatggtggc cacccgcctg tagtcccagc tacccaggag gctgagcagg ggaattgctt |
| 15601 | gaacccggga ggcacaggtt gcagtgagcc aagattgcat cactgcattc tagcctggca |
| 15661 | acagagcaag acatcgtcta aaaaaaaaga agaagaaaaa agaaaataca tagtaaaatt |
| 15721 | tattggcttt ttaaatggat aatttcatat agtcagtcag tgccggctta gatccatttg |
| 15781 | ttagagaaca atgctaatga gacctcttga tactttgtgt tcctcttatg ttttcctagc |
| 15841 | cttgacgtta gcaactaaca atgataacta acattattga gctcttatgt gccagggact |
| 15901 | attacaatag attttcaatt attatctcat taaatcttca tagcagccct atgagctaga |
| 15961 | tactgttact acctatatct taacagatga ggaaatttga aacagagcag ttatttgcca |
| 16021 | agagtttcag agctattaac agaagcctaa tacatgttct tggactttgc agaaataatt |
| 16081 | tgttgatcca tgcggtttca ttgctactgc ggaaagtaag tctcttggcc attgtgatgg |
| 16141 | gtcaaaatct ttgtacatga agggtatgac tgctgttcca ttgtatccag agagtgaatc |
| 16201 | ttgtgattga gttgggattt gaggtggcag tgtatactaa gtttgaaaag tatattatca |
| 16261 | tatgttagct ccctttagaa attttcccca agaagagcta ttacattttt taatattaaa |
| 16321 | aaagcataag atatggccat aatacaatta gttccacaag ccatacacac ttttttctta |
| 16381 | tggcttaaaa ttttattcat gtttactact ttgcaaccca ctgatataaa ttatttcaca |
| 16441 | gatcctttat tcaaatgaaa ttctcagtga gtctgagaag atagtcttaa acacaagcta |
| 16501 | gctgagttta caatagagat tatactataa atggtaactt caatgtttat tgaaaataaa |
| 16561 | ttccttaaaa acaggtcttg tttcattcat ttaaagagat tatcaatata tgactgttaa |
| 16621 | atttttatag tctccctgtt cattttctga tgaacacatg acagaaaatt gaatgtgagc |
| 16681 | tggaaataca gcagactatg agaaagcatg ggtcatgtat aatcacttta aatttaggct |
| 16741 | ggtgttttgt attcatctat aatatgacca cctcagggag ggatgtaagt aaacattctt |
| 16801 | tttttagtta tactttaggt ggcttaatta ttttaaagaa tatgttattc aagaattgtc |
| 16861 | tattgtactg ttttaaagaa taagaatata attcattacc aactttgaat gtcttggatt |
| 16921 | cttagtacta cctttttctt tatcatgact agtaggatca catcataatt tggtactgtg |
| 16981 | tgtataaaga tgaagtgtgc ttacctttat taccaaaatt cagaaatgaa aatgtgaatg |
| 17041 | ataaaagata agggatggct atatattttt ttgtttttgt ttttccaaac agaatctcat |
| 17101 | aacttaccag gctttagaaa aacatctttg cagtatgcca tcatacctca gaatgttaag |
| 17161 | tagataggaa ataaaatttt cactttttgt aaagcagtag ggtattatcc tttctgacag |
| 17221 | cctgtaagac tgttttggtt gccaaggggc caatcttaga agtggcagtt tttgttttgt |
| 17281 | gaccaaccca cagcttaccc tttgttagtc catatctgtc atcttcattt tctctttctg |
| 17341 | cgtaaatgct tcttgataca gtaagcaggt gtttcaacta gtcagccatc tagtgtgatc |
| 17401 | ttttctggtt tgctacagtc agtaaaataa gtagctctat aagtgtaaat agtattactt |
| 17461 | gttttatgta gtaaaactcc aagattaagt tagagatatt tttggctaga ctacattgaa |
| 17521 | tgcattgaca atggatttcc cagctcccag aaattcgttt gtgtctgaat tactttatac |
| 17581 | ttggcttatt gtgtagagaa aactttagaa aaagttcatc catccatgaa tgttgaatga |
| 17641 | atgagcagat taataaataa atagctcagg gaaagaaaat gtgtgggctt ttagtcataa |
| 17701 | ctatgttttt tctcttttgt tcccttcctt atgtgccatt tttcaataat gggagtgtgt |
| 17761 | atctaatgat gttgataaac tttttaaagt cattgatgaa cagtaatctg aaaatcaagt |
| 17821 | tttgattaag ccgtagattt cttttagggt ccaatatttt ctttttagaa atccactatg |
| 17881 | atatttttgt agggataaaa gacaaccatg tactctatag atggaaaatg gtaggataca |
| 17941 | gtttgttggc cggccaactt ctctgaaaat actttttcct gcatagaaga taatcagtgt |
| 18001 | tgacaaggga gaagccaagg tataatggct gttattcatc tgtctcatgt attcgtcaag |
| 18061 | tgtcatgtgt taaatatgaa caaaaaagac ataatctctt tcctcctgga tcttaaaatc |
| 18121 | taaagtgaga gataattcac cagataaatg ctgcgataag ggaaatagag gattctatga |
| 18181 | gcttatgact aaggatagat ctgtttcctc agatgtggaa ctaagaagtg gcagtagttg |
| 18241 | agaagttcat tttgggccat gcgatttgag gtgtggctat tcaagtatag gaggtaggtg |
| 18301 | gttacacatt ttttaagcta ttaaatagaa ctttcacttg aaaaggtttt tttctctctt |
| 18361 | tcctcagcat ctatctccct acatacacaa aaaaattgca tgtcacaatt catgaactca |
| 18421 | gtgagtgact ctattttgaa aaatgttaga ttatatcaac tgaagtctgt tagaggcaaa |
| 18481 | tgcttttggt catttttttc agcagggaca ttccatatgt gtaaaatatt tagaatcccc |
| 18541 | atgtatttat atataaatga atcttttacc atcttaaatg tttaataagt tatatttttc |
| 18601 | ctttctttca acttaagtat acaaggttat cagcaaacat ttgtagatgc tctatgtgag |
| 18661 | gtgttttgtt tattagtttt aaggaagaga aaaaaggatg gattggatca ggaagtcagt |
| 18721 | tattgttttc tgggttagtt tgagtccaaa tcctcaaata tgcggcgatt tataagtagc |
| 18781 | aaactaggaa tttaatccac ttcagttttg gataacttgg ataagacact ttctgtctat |
| 18841 | agatctgtct atagataact tggataagac aagttatcca aaactgataa atctgaatta |
| 18901 | gttaacagat gaggcaaatt gagattgtaa atgctaaaag attattagaa aactttataa |
| 18961 | actaaatagt tctatgccat gtctttttag tattttgctt gtactataat tgtagtgaaa |
| 19021 | ataggtcagt atctaatttt tatagtcact agagctctcc aactaaattt agcttagatg |
| 19081 | tttttctagc aattatatac ttacaaagtt tacttttaaa aattgtgtaa tacatgcctt |
| 19141 | tatagaaatt taagaaaacg acttatggat tatgtacttc agatcacagc acaactagaa |
| 19201 | gatgttgata cattacataa aaaatcagga aacctatgaa gaacatcaaa agtggcaagg |
| 19261 | tgtgttcact tgcactttgt gagattgaaa ctgctgccat tatctgtgca aactatggtt |
| 19321 | tgagtacttt taatttagca aagactaatt attttttaaa aattttgggg taaaaaaaat |
| 19381 | ggtctttagc gtagaagagc tttacccgac ttactttagt tggttcaatt ggttgatttt |
| 19441 | ggtttgcttt ttgaaatttt gtattgtctt aatttggggc acattgaact caataacagt |
| 19501 | tcagttgcat tataattcat ttgggtaaag tttattgttg gaggagggta agtaaatatt |
| 19561 | taactgaaaa agtgaagtaa atatttattt aaggctataa gttgaaatat ttgtcagctg |
| 19621 | agaaattttc aggtaacttt tttaaccttt gaaaaaaatt tactatagca tgagtatttt |
| 19681 | ttatactgct atgtataatc catagtcacg ttttttaacc tttttatata aaatactgat |
| 19741 | acagaaaacc atacaatcat agtaatcttt taaaattgct ttataattca catagcgtaa |
| 19801 | cattcaccca tttaaagttt acagttcagt agtctttagt atattcagac ttatgcaacg |
| 19861 | attaacacaa tttaatttta gaacattttc atcacctcca aaagataccc tgtactcatt |
| 19921 | agcagtcact ccccattcac cagtctccca gcccttatca accattaatc cattttctgt |
| 19981 | ctatagattt gtctagttta ggtattttat ataaatggaa tacaggtaac tttttattag |
| 20041 | tgatttttca cttacatgtg tcttgcatgt ctcatgagat agctatgaat aagttagcct |
| 20101 | tcttttacaa cttaaataac aattcttgtc tgaccgggaa aaagtattaa gttattgaga |
| 20161 | gactgcatcc caatttaaac caagaaccag actccttact agtattataa ctcaaaagaa |
| 20221 | tgtgtaccat tagcccttac ctctggtggt gcttttcact gtgaattgtt cagcagcctc |
| 20281 | tcaacttccc gaaaatacca cttaagtctt aaactcagac ttgcatgagt ttactgagat |
| 20341 | atgtttacct ttgcagctgg cttgcctaga gtctaccttc ttactaccct actaagtcca |
| 20401 | gaatgaggaa gaattaaaag caagaaagga gatacagaag aagagattgc tcaggctgag |
| 20461 | gaggggcaaa gtgataagag caggattaga aagagacatt aaaaattcaa ggatactgag |
| 20521 | tttgttggca tcagaataaa tcttggaggc tgctttaaga gggaataaaa cctagaggga |
| 20581 | aggaataaaa ggcaaagatg ttataaaaca taggtaaaat atttaaaata ttttgtcctg |
| 20641 | aattttgaat tttaggcata tgtcagtgaa gcacaaagga tagccccctt ttttgatgag |
| 20701 | ctttagtatc cttaagatat tttctttcta gaaccttgtg taggaatttt acagctgacc |
| 20761 | ataatcatgt gttctctttg gtttgttcca acatctttgg attggaacca gtacagagta |
| 20821 | ggaaacccat atcaccttgg gattattttt cagataaatc ttccagagga ttaaatggca |
| 20881 | ccatccccgt caggtccaag taggagggga agaatcctat tttgggtgct gtcagattca |
| 20941 | gtgggctgtt tagttcactt tttatccaga aaatggagct aaatgagaaa ttgcagcttc |
| 21001 | caaagaaaat cataccttga ttttatagat caaagccttt ctgattagat cagaattgtt |
| 21061 | cctgagattt taggggcatt cagttaaagt gccccaagaa gagtgataaa tcacatatag |
| 21121 | gtgtaaagtc aataaagtcc tatagtaagc agactggaat ttagggggag gggaatacac |
| 21181 | actgttagac tgttggtacc tatatttgac tactaggatg attatatatt ttaaattctg |
| 21241 | tattcagttt cttatcagaa atactgccta caatatctta tacttaattt tttaaggaac |
| 21301 | aggtgataaa gataggttat attttcttca gaataattga tattcattat ttaatagata |
| 21361 | aaatgtgtta tctgaatgtt tatagtagta gaaaccaagt ttacaaaatg tctatatata |
| 21421 | atttgttttt aataaccaag gattctttct ttacaaaaga tttcctctat ataaatcttc |
| 21481 | ctgttttata gatgatctgt attataggag aaatgagtat ttgttgattt aactggctaa |
| 21541 | tctacaggac tgagcattat tagaggagta tttaaacata gaagtctgtg aaaatatttt |
| 21601 | tagtgctgag ggaaattgga gggactttct agaaccgcaa actaatagaa aaccaggcat |
| 21661 | agtggctcaa gcctgtaatc ccagcatttt gggaagccga ggcgggagga ttgctgaagc |
| 21721 | cctggagttt gagaccagcc agggcaacac agtgatacct tgtctctaca aaaaaatttt |
| 21781 | taaaattagc tggacgtggt ggcacatcta tactcccagc tacttgggag gctgagacga |
| 21841 | gaggatcacc tgagcccagg agctccacgc tatagtttga gttgtgatcg tgccattgca |
| 21901 | ctatagcctg ggtgacagag caacacccta cctcaaacaa acaaacaaca aaaaaaccct |
| 21961 | tctacctctg aacatgtggg gatttaaaga ttgctatgat ttaaatataa atcatgaata |
| 22021 | tagaaaatat gagaacagaa gagtatttgt taaacaagac taagctgtgc tgcataaagt |
| 22081 | aaggcactat cattgtgctg gtattttctg attctcttag tgcaataaat gttaaacttt |
| 22141 | actactacct tagccatgtt gcagggaatt gacatgaata gttcctgggt tttggctggg |
| 22201 | tgcggtggct cacgcctgta atcctagcac tttgggaggc ttaggtgggc agatcaggag |
| 22261 | ttcaagacca gcccggccaa catggcgaaa ccctatctct aataaaaaac ttaaaaatta |
| 22321 | gccgaacgtg gtggtgcacg cctgtagtcc catctactca ggaggccgag gcaggagaat |
| 22381 | tgcttgaacc tgggaggtgg aggttgcagt aagccaagat ggcaccactg ctcctagcct |
| 22441 | gggtgacaga gggagactcc gtctcaaaaa aaaaagttcc tgggcttcaa gggcactcta |
| 22501 | atgtgcacat cttaaattct tagagcctac ttctatggac gatttatgct actgttgatg |
| 22561 | gagaatttga ttttcttttt tatcttcccc ttccccttgg cctttgtagg ttttttgaag |
| 22621 | tgaacatact tttatgtatt tacttattta ttgagcagtt cccaaatggc aactttatta |
| 22681 | ttttatttct tcaactcagt tgtctagttt acttcatttg tatctaatct atttaactca |
| 22741 | tcctttgagt ttttaatatc aacattttaa attttatttt tttagcttca aaggtgtgca |
| 22801 | tatgcttgtt acatggatat agcatgtata atggtggagg ttgggcttct agtgtaccca |
| 22861 | tcacccaaat actgaaggtt ggacccaata ggtagttttt caaccctcac cctctcccac |
| 22921 | ccaattgttt ttatttctga aagtaatatt tctttcttgg tctgcctgat aattttttta |
| 22981 | tagtttctta tttttcgtta gttttcaaaa aataccttgt tgggcattct gtccatagat |
| 23041 | gtactatctt atgtttgcta attccaatat cggaagtcct taggaagttg tacttaaggt |
| 23101 | agtttgtttc tttctgtatt tggtgatttt ttttatgagc taatatttga ttaaaccgaa |
| 23161 | tatttctcca tcctctgtat agttgcctac aatgtaattt ctactttttt ttttttagca |
| 23221 | taaaagagtt tcattttcct ttttatactc aataacttgt ttaattagcc agtattttta |
| 23281 | ccagtttctt tacttattgt tccttgcatc ttctctgagt ccacttttct tcttgctgaa |
| 23341 | gtacatcctt tagtaatcct tttagtaaga ggctggcttg taaatgtcct tagtttttgt |
| 23401 | gtgaaagtat attttatgca ttactttgtt tttcagctag atagggaatt cttggttgat |
| 23461 | aggtttcctc ccatcagcaa cctcagtata ttatttcatt gtcttgtgaa acttggcaaa |
| 23521 | cttggatctc tgagaagttc gatctctgcc taattgttat gcttctgtat gtaatttttt |
| 23581 | ttttctggta gttttttaaa agatttcttt tctttattct gtcaccacta cttactgtgt |
| 23641 | gaccttgggc aagttcaaaa tggatataaa attgtatctg tgtcataggg ttgtaatgag |
| 23701 | gattaagttt taaagcactt agaacagagc cttgcatatt ataactgtga catgttaata |
| 23761 | tttgttttat tttgccatac ttgatattct aaacacattt ctatgatacg cttaagtatg |
| 23821 | ttgttttatc catagctcct ctttgaaata cattgttgat gggaagattc atatctttct |
| 23881 | ttgtttctag aaaattcctt agccttcata tctttgaata ttcattttcc aatattcctt |
| 23941 | cttagtctct gtattatgtt ctaggtggtt cctcagtatc attttctaat ttactaattc |
| 24001 | acactttgac tttgtccagc tagagtttat gtggcctcag ttccagtgaa ttttttttaa |
| 24061 | atattcaggc tgtctctcta atagccacct attctggttt gatttctgcc tatttttaat |
| 24121 | ttttcttcct ttaatgacta ttgtggcttc ctcttaacat ttgaaattat tttcagattg |
| 24181 | tcattttttc ttctagaatg aattacagtt gttgatttta ttggctgtcc atcttagttt |
| 24241 | agttttcttt gtgtgttttg agtttgcttt tgcagtctcc tattgactgg aagtttttgt |
| 24301 | tgtgttttct ttttcactct tcttatccag tgcttttaca tgcacctaca tctctgtcta |
| 24361 | aaaccacatc tttatattgg aggcttgatt cccttgattc ttggtaacat caggatcagg |
| 24421 | cttctctttc caagaataag acacaaacta agatctatat ttcttctcac tggacacata |
| 24481 | atttcatcta aaccgtagcc ccatgcaatg gttagtagta gctttttctg gctcctttca |
| 24541 | ttggttggaa gagcctctcc accctaggta ttggtttcaa gcagtgagcc tgagttctat |
| 24601 | cccagccatg catgggtacc tttttagtcc ctgttactat ccctacagga tgtggacttt |
| 24661 | tggccatctc tgttacttga gcccagaagg ctcacagttt tagcttcaaa aactgctttg |
| 24721 | tatttaaaga gttcagtggc ttggtctttt ttaatatttt gtttgtatag ttaataaata |
| 24781 | tttaacatac attaaaatgt ttggaacaga tacatttcaa aagtgaatat acaaatccat |
| 24841 | ttggttggaa gttagtccat tttgtgttgc tatcacagaa tatcacagac tgggtaattt |
| 24901 | attaataata gaagtttata tagctcttgg ttctgagggt ggtaagtcta agattgaggg |
| 24961 | gctgcgtctg gtgagggcct tcttgccaga tggaaggcag gaatgcaaga gagcacaagt |
| 25021 | gcaaacaagg gcaagacggg gccagactcg attttaccag gaactcactc ctaagataag |
| 25081 | ggcattactc aattcatgag ggcagagcct tcatcaccca gttactttct atggactcac |
| 25141 | tggttattac tgtcacaatg acaattcaat ttcaacatga gttttggagg tgacattcaa |
| 25201 | atcatagcag aagtcaatgt ttaaaatcaa tgtatatcct acataccctt cagaaagata |
| 25261 | ctagttgact ctcctactta tgagaggtat atttttttat tcctttgcca atattgggtg |
| 25321 | taataatttt ttaatctttg agaaggtaat gtataaaaaa caaatttgtg tttaatagct |
| 25381 | agcaggattg gtcattgctc catatattta ttacccattt atattctttt atttatttaa |
| 25441 | ttgcctgttc acattcattg cccatttctt ttcctttaag gtgttcattt ttattgtctt |
| 25501 | ttaaaatagc tgtttgtata ttgtagtaat gttaactttt gtgtatatat tgcagatgct |
| 25561 | taaaagacgt ttgcttttca ggttaatgag ctttatttta tagtgtagga gtttcttttc |
| 25621 | tctggagtca ctttttctta ttcctttgga atcagtcttt tcttgtcatc cttagaaagt |
| 25681 | cctttcctac cataagatta cagaaatatt tgcatacatc tactggtatt gttttattat |
| 25741 | taatacttca gacttcaatc tttaatctct tggtatttat tttaaggtgt gatgttgaag |
| 25801 | ggattcagct acccaccact ggagggcagg agtggaaaag atcagctttc aggcacccat |
| 25861 | tattgaatct ctctgttccc agagagctct ttctggattc tcagttctat tccactgata |
| 25921 | aatttattcc tgcattagta ttgcctctta aatactataa tttggaatca agtttaaata |
| 25981 | tcaggtattg cagattctca tgattttaag aatgttttgg ctattcacat ttattcatat |
| 26041 | atattttgaa atgatttaat caaggtctgg gaacaaaaag gtccagttgt gattatattg |
| 26101 | aatttataga ttaaataaag ggaggataac catcttcaca attttgaatc ttcctatcca |
| 26161 | agaaaaagaa gtgtgtgtta ttctgaagcc ctcttcttaa aaaggttcta tttcttgtta |
| 26221 | tttatttaga tatactttta tttctgtttt aactttctaa gtgattactg ttaatcattt |
| 26281 | tcatatatgg ttttctatag tcaaataatc ttattgagta tttttttccc tatcatttca |
| 26341 | ggttttgagt ttgccatcat gtcattaact gcaaatagtt gatctcctta taattttatg |
| 26401 | cctctttttg ttagccttgg cttatctcaa taaccattat ttctacacat tgctgagtta |
| 26461 | cagggataag agttggtgtc cttgttcttt tttctttcct gggaattgtt ctagtactga |
| 26521 | gatttcctct ttggcttgaa gtaagtatta tattaaggaa taaattttat caaatccctt |
| 26581 | tcagaatatg atgagatgat catatggatt gtttttaact tttaaaaaga tgacttacat |
| 26641 | tagtcagctg cctaatgttg gctcattgtt acgttcctaa aatgatccct atttggtgat |
| 26701 | ttgtaatata tattttactg actttttggc atctgtatta ataagtatgt ttggactata |
| 26761 | gttttatttt tttagtgctg tctttgtcaa attttataga aggattatga taggtttgta |
| 26821 | aaaagaattg gaaatttttt tcctgtaatc tatagataac ttaggtaatt tgagagttac |
| 26881 | atgtttcctt agggcttgaa ggatcttgtt ttgaaatagt cttgcctgat gcacttttat |
| 26941 | ttttaatagc ttattttgag aacaatccaa atttaaaatt tgcaaaaata atacagtgaa |
| 27001 | cttccatatt tatttaaatg gccaattgtt aacatttttc cacattagct ttacagactt |
| 27061 | ttttccctgt gtatgtgcat atatgtattc atattctctt ggcatgcaca cacacatttt |
| 27121 | gttatttttt agttttgctg aaccatttaa gactcagtat ggacatcatg aacctttact |
| 27181 | cttaaatatt taaatgtgtg tcccctaaga acaaggacta tcactttctc ttacataacc |
| 27241 | atagttcaat tatcaaattt aggaaattta acattcatac aatactatta tctaatatac |
| 27301 | aatccatagt cagattttat cagttgtccc agtaatgtcc tttataacaa tcttcccctc |
| 27361 | cccattcctt caccctttgc cttcatccaa gccagtccat aatcacatat tacagtttgt |
| 27421 | tgtgacctcc ttggtctcct ttaacctgga gtggttcttt agtctttttg cattttgttt |
| 27481 | ttcatgagct ctgaagtttt ttgagggata gtcctttaat aactttctca tattgttcca |
| 27541 | ggtttctggt cttcactcta gcactaattg aaaaaaaaaa aatttgtttt ccgagaactg |
| 27601 | ttactttcct ccatattttc aaatttatta acagagaatt gtacagagtg atttatttta |
| 27661 | caacaataaa attctatttg tgaggtgagt atagcctgtt tggcttctaa ttttaaataa |
| 27721 | tttttttagt tagactattt agtggcttac ctattttatt tttttcacat ataacatgta |
| 27781 | ctataatttt acgtaatata ttccttcagc ttctatttta ttttgtatgt gtgtgtggtt |
| 27841 | catttttctt tttaggttga atatttagta tactgatttt ctttagtata ctggtttttt |
| 27901 | taactaagtg tgaatttttt tttttctatt ttcaacacac tattgctggc ttaaaggtga |
| 27961 | ggtttcttaa taaggtttgg tcacatctca gaatttgata ggagattttt ctcattattg |
| 28021 | tttctaaata ttctataatt gtagaaatat gtaaactcaa tgcaaaaaac tggtgtagga |
| 28081 | gcttactttt taagcaattt ccacgtttga ggctttcttc tttctttaca cattatcaat |
| 28141 | atctactatt aatgtataat tcagatgtgt catttcatta tttcattttc tttttttttt |
| 28201 | ttttggtctg tggggtagga tgggggagtg tgttagaaat ctataagctt ttctttccaa |
| 28261 | catataatcc aataattatt tgatttttca attaatgttt taagtgtgtt tgaacagaat |
| 28321 | atatagtctt gaaattttgg accacatctt ttaccaattt aaaacatggc tcttattctg |
| 28381 | tttattgctt tattccttgt gatttacttt gtctgaaatt gaaattgcaa ttcttggtat |
| 28441 | cttttttgcc tagtgtattt cgaccacaac aaagtacttt tatactgaca tctttatttt |
| 28501 | tgagtttctt ggttttgatt agtctcttga ttgcatgaat ctatctttaa atagtttatt |
| 28561 | ttttcagaaa tgaaacatgg ataacactac tttagtgctg gcatatttga tcatgtcttc |
| 28621 | ttcatgtctt cttattgcgt ttacatataa atgacaacct tgctggtcgt agtattcttg |
| 28681 | gaataaaacc tctttatcct caacgtttat aggcattact tcgttgccta tcatctgatg |
| 28741 | ttgcaaagaa gtctgggacc agcctgattt ctcttttctt tttcttttct ggcatgttta |
| 28801 | ttttattttt tttatttgag acagtcttgc tctgtcaccc aggctggagt gcaatggtgc |
| 28861 | gatctaggct cactgcagcc tccgcctcct gggttcaagt gattctccta cctcaacctc |
| 28921 | ccgagtagct gggattacag gcgtgcgtta ttatgcccgg ctaattttta tatttttagt |
| 28981 | agagatgggg ttttgtcatg ttggccaggc tgatctcaaa ctcctgacct caggtgatcc |
| 29041 | tcctgccttg gcctcccaaa gtgctgggat tataggcgtg agccatcgtg cccagccgat |
| 29101 | ggcatgttta ttttgtctga attgtattct taaaatttgt aatcttcaaa ttatgcttcg |
| 29161 | gtattggtca gtttctacta aatgtatcta gtacttgata aacgccattc aatcaccaag |
| 29221 | ttcagtcttt agtttatgat ttatttcttc tgcctttgaa atttaatttt tcttccatta |
| 29281 | tttggttttc ttatttacaa tgtgctaatt gagctgttgg atctgcatcg tttgtatctt |
| 29341 | ttctcaagtc tacttgtttt tgtttcttta tgcttacttg catactggat gatttttgtc |
| 29401 | ctgttcgttt ttaccccttg cagattgttt gctgtagtgt cttcattcct cattattgct |
| 29461 | tttcatgaca tttaaaatat tgcaatgata ttttttctct gaaatatctt tttttaaaag |
| 29521 | ctctgttgat actttttata ttctggtttc ttttcatccc tgtttcacaa attaggtttt |
| 29581 | ttaaaatttt cttatatgta agttgtcacc tagttttcta aggtaaatct tttagactgt |
| 29641 | gacatcatgt tcttgagtac ttttttagtt acttttttat ttcagaattt ttgtatgggt |
| 29701 | tctataaatt tttttctgct tttttactta aagggtggca gttttctctg ttgtttgccc |
| 29761 | aaaaataact agctactcct tgcctgttct gtcattatac taggatacca ttacctcttt |
| 29821 | cccccttgga atttaacagg ctgaattata ttaaattatt ttagttatca tatttcatta |
| 29881 | tttttgagag aaatgatagg gattttgtcc tattaacctg ttacaggtaa gaaactttgt |
| 29941 | ctttcttgat ttttctcgat gcttgttgaa attactgatt attttagtta tcatatttca |
| 30001 | ttatttttga gagaaatgat agggattttg tcctattaac ctgttacagg taagaaactt |
| 30061 | tgtctttctt gatttgtctc ttgatgcttg ttgaaattac tgcacatttt catctttaca |
| 30121 | agtttggcag attttctttc ccaacagaag aacttgacta ttgggcattc tgctaatttc |
| 30181 | catggctgct ataacaaatt atcacgaact tggtggctta aaacaataca gatttattct |
| 30241 | cacagttctg gaggccacaa gttcaatatc atcttcacgg ggacaaaatc acggtgtcac |
| 30301 | caagaatgtg ctcactcccg aggctttaag gaggaatctg tttcttgctt tttttcacct |
| 30361 | tccagtggct gtcagcctca tcacttgcct ggtagccaca tcactcgaat ctttaagacc |
| 30421 | agcgtcttca aatctgtgtc tgctctgtct tcacatcacc ttctctgtgt gtgtcagatc |
| 30481 | ttcccctgac tctctattct aaggacatgt gggttcactt agaggtctct ggataatcca |
| 30541 | ggatcctctc tccatctcaa gattcttaat cacatctgca aagacctttt tttccaaata |
| 30601 | aggtaacatt tataggatta ggactggtat ctttgggagt cattattcag cctactaggt |
| 30661 | atcattttgt tttgctactt tagagtgagg cagacatatt agtcaaaaat tggattagag |
| 30721 | taaagcctca tagattgtgc tgtttgttag aatcacagat actgtcctct ttaccgaatg |
| 30781 | atgcaaactc tgatgccatg gtggaaaagt atatgaattg aattggatgt ggtcaataag |
| 30841 | gattgctagg gactcgagaa tgcatgttgt tctagggaga ggaaatacgt gaaggtcaaa |
| 30901 | ttcaggttat caggttatca gtttgtaacc tagtttgtag ctgctttttt cttttcattt |
| 30961 | cttacaagct ggtcttgctg tagtaaaata atcttgtgtg tctcttataa tcttgatatt |
| 31021 | gagaataaga ggtttttttt ttaatgggaa ttccatccac atgattttaa agtattactt |
| 31081 | tactaatgat atttacctag gtattcctag aactttagtc ttctcatgta acattttata |
| 31141 | catttctcag aacaaggtaa ttgacaaata atcaaggttg cttatttatt gatgaggaaa |
| 31201 | gttagacatt aagccaaagg acttgtccag tatcgcatat tggatagacc tataattttt |
| 31261 | aactttaatc ttgtggtgac aggtatttcc attttttttt ttttttgatg tttcatatgt |
| 31321 | gctttgatgt tgatgtgtca tgtgacatga agtgtctccc tattgttgga taattgggca |
| 31381 | agtctaagtt ctttttactt tatattcctg attttgtttt tctagatgtg ttatttttac |
| 31441 | agattcaggt tacttctttt tttaaatcat gttattagag ttaaactaat gaaaaatcat |
| 31501 | atagagatga acttggtttt gaaatataat gagtagatac ttgcttggct ggaatttttt |
| 31561 | ctcacaagtt ttttataaag atggtatgag tgtatacttt gtatccagga atgtgtgtac |
| 31621 | actaggggga gtgttggggg gatgagggtg gaagacattt taagacacac aggaggctgg |
| 31681 | tgtggtggct catgcctgta atttcagcac tttgagaggc caaggcaggg gaatcacttg |
| 31741 | aggtcaggag tttgagacca gcgtagccaa catgatgaat ccccatctct actaaaaata |
| 31801 | acaaaaaatt aactgggcgt ggtggcacat gcctgtaatc ccaggtactt gggaggctga |
| 31861 | ggcacagaat cacttgaagc cgggagccaa gatcatgccg ccacatccca gcctgggcga |
| 31921 | cagagtgaga ctctgtctca aaaaaaaaaa aaaaaaaaaa aaaagacaca gaagacccta |
| 31981 | agtgtacact cagatgctct ttatatccat atccattatc agtttggtga tacgttattc |
| 32041 | tagtttcatg aacctttact cttagaactt ggcttattct ctaatatcct ttggattcag |
| 32101 | ttttgcagtt gagatggcta atatcaattt gatactcttt ccttttatga ttgctcatgt |
| 32161 | ttttgtctag aaccttgcat tatagtattt ttttttcttt tccttgcatt tctatagttg |
| 32221 | tgaacaacta ggagtgggtt tttagcaatt aattcagtga gcttgttaat tttgaaaagg |
| 32281 | cttcaactga ggaacacttt cttctcattt ctttgatgat agcttctctg tttctccttc |
| 32341 | agaaagtctt gttttttata tgcatattta ctttttgggt gcactctcct tgttaatgag |
| 32401 | ttatttgcat ctgattttga gtttattcag caaatattta ttaatgattt actgtatact |
| 32461 | ctgtgcttgg ggagttgacc tagggaactc aaaggaggct cctttgaaga attgatgctt |
| 32521 | gagctgagat ctcaggaatg agtaggaatt atctaagaca tgagagaaca gctgttaagg |
| 32581 | tagggaaagt agcatttgtt tatatatttg gaggaaggaa gtatgatgag tactaggtac |
| 32641 | tgtaagaaaa cccttgtggt tggagtagag aatgtaggag aagggacagt atgaagtgag |
| 32701 | atgcggctag agaaggtacg taggggcttg acttcccagg gccttcatgt gagccatatt |
| 32761 | aagcagttat ttatccacct cctaccccca gcagtggaaa gccattgaga aacttttagt |
| 32821 | acagaagtga tatggtgaaa tttgtatttt gaaagattta cttaggctgc agtttggaga |
| 32881 | atgaattaga gggatgccag gatccacgtg aagagactaa agacgctagt attctgcagt |
| 32941 | gagaatgatg gtaacttgag accagggcat tggtagaaaa aagtggacag atttgagaat |
| 33001 | gcttaggaag catataaccc atgggatttg atgatggact gatgggagat gaggtagaga |
| 33061 | gaggtatcaa agatatcctt agtttctgtt tctttttctg agttggggtc ttgctatatt |
| 33121 | gcctaggctg gtcttgaact cctggattca agcaatctta ccgccccagc ctccaagcag |
| 33181 | ttgggactac aagtatgcaa caccgcaccc agtgaatgtt cttggtttgt attcaccttg |
| 33241 | gtgggtgatg gtgccattca ctggtaaaag ctgcagatat gtgagggtgc tcagggaggc |
| 33301 | agcatagaat gggaaaaggg cctacatgta acccttgagg aatgccaatg tttaatggct |
| 33361 | tgggagatga aaatgagcct gcaaaggaga cgacggaaaa gcttaggggt aggaagaaag |
| 33421 | ctgggagtgt gtgtgtagca tcttgaaagg ccaatgggaa gagtgtttct ggagggagtg |
| 33481 | ttatagttga atgcttctgt gagcagtcaa ttagaggagg gctaaaaatg tcccttagat |
| 33541 | taagtgactt ggaggaactt gatttagtaa aatggccaga cagccttttg gagcatcaac |
| 33601 | atttttttta agaagtttaa ctgtaaagag aagagaggca catgggtagc tcgaggggaa |
| 33661 | taagtcacat gaaaagttat ttttaatggg taacatgggt gacacttgcg gctttaaaag |
| 33721 | tcacactcca actgagaaat gttgataaaa aggagaaaaa ggaaaatagt gtaacattcc |
| 33781 | tgaaaaaact gtcatttaag actcctttga aacatgggca ccaaagctag tctagtagag |
| 33841 | gatttattat cagtgagagg acagagtcct attacagcag aaaagtagga ggggagggtt |
| 33901 | atgaaagaat tcattgaaat ggttcactca tttattgatc ttttttttct gtagtatcca |
| 33961 | atctgccatt cattgtctct attgcaattt tcagttcagt aaattgcttc tcatttccat |
| 34021 | gaaactattt tcagagtacc tcttttttaa ttgacagtct gcaattgttt tattcattca |
| 34081 | atgtcatttc atatcttggt tagtttctaa ttttgagcta atgaatactg agtctccctc |
| 34141 | ttttcaactt ctgaattaaa aaaaaagtca tcttattttt ctctgttcat ttttacactt |
| 34201 | tatttagcct aagtatctga aattggcttt gctagaaatt acaattagct cactgttgtt |
| 34261 | ttctttcagt cccagacaaa aaagaaaaat tttagatttc tttcttttct cttcttcctt |
| 34321 | tttttttttt ttaaggtcat ttatctttgg cagttttaga gatttagaaa agaaaatata |
| 34381 | tagacttgta ggcagaagaa gatatggcac tgagggaagt tttagtttcg ttctgtgaca |
| 34441 | gtcttcctta tcttgtatgt tgtctttaaa aagcctacac atttagattt tcattacctc |
| 34501 | atttgcaagg atacaggata gcacatgtag gagaattgga gtgtaagtcc agaaacctaa |
| 34561 | gtcttattct tagccttact gatgaaccag ctttgtaacc ttgggcaagt tattctgttt |
| 34621 | ctctgcacct tctcatttgt aaaataggtt tacacaaatc atttctgcat tttggctctg |
| 34681 | attcattatg caagttatca aaatcaaatt tgtaactata aatgagtaat attttattat |
| 34741 | ttttattttt ttagagacag agtcttactc tattgcccag gctggagtgc agtggcacca |
| 34801 | ttataggtca ctgcagcttc aaattcgtgg gcccaagcag tcctccttcc tcagcctcct |
| 34861 | gagtagctag gactacaggc atgcaccact atgccccgct aatcatttct acagagtcac |
| 34921 | gctattttgc ccaggctggt cttgaactgc cctcaagtgg tcctcccacc tcagtctccc |
| 34981 | aaagcactgg gattacaggc atgtgccacc atgtgtggct ctaatcattt ttaaagcaac |
| 35041 | aatcagtggg aaatttcagc tccaaatggg agaaaaattt ccaaagatta gccttttttt |
| 35101 | tttttttttt tttacagatt tgctacagtt ttgttaatgt gcttatttct cattaccata |
| 35161 | gataactttg agatggctgc cttggattac aaactagaat ttattactat ttcacatagc |
| 35221 | ttgggaaatt aagtgtagtt tgtgtagcct aaattatcct ttatagagat gtagctgcaa |
| 35281 | gggttagcat ttcttctttg aaccccaggt atgcagtata ttgcttaata ctctattgtt |
| 35341 | ccataaatat gcattccaga agtcagctga ccatttgtcc ttatctttga ggacaaagcc |
| 35401 | cgtacatatg taagcattag tgagcaatgt agaatataaa gatttggtag cagtagagag |
| 35461 | tttacgctct acatcccact gcaagtaatt tgaatattgc catattatcc actgtgacct |
| 35521 | gtgctgtttg ttaacgttcc ttttccccac cttccccgag gagcttgtag tctctcacta |
| 35581 | tatatttttt tctctttgga tttcccttca gcctttagat ttcttatctc taatgcaagt |
| 35641 | ctactctaga tttttctccc aaatgtttaa aaccaaacct attatctcag tggtcatcaa |
| 35701 | aggtcctcta gttaccagta tccacccttc ttggaatgcc tgtttagcaa tgttcttttg |
| 35761 | ttatatgagt aagaataact ctggtatttg ctaagagaat ataattttct ccacattgta |
| 35821 | tgtatctatg tcctttgttt ataaagcctg gggggcgttc cctattaggt taagtgtccc |
| 35881 | tacgtaaata gaaaaatatg tagggtttta ttactcaact ttttaaggct tctgctcaaa |
| 35941 | ttgtcagttg aataactctt gttactttgg ggctaacatg ttgcataggg attttaacaa |
| 36001 | taaaatatat agagcatttt aaaaattatt cactataatt ctgagtggat taaaccaaat |
| 36061 | gaccgggttc aagactctac tatgagttca cctttgctta ataaaaggca agactgaaca |
| 36121 | ttaattaatg acttcttggc ccagccttag aatttttcag tttagggatt agtttggttt |
| 36181 | ttaaaaggta caaaatgttc atgaaatttt ctgattatat caaatattaa ttcagtcttg |
| 36241 | ttttttgtca tagtactttg ttttgaaaag ttcagatcaa ctgataatac tagtgctgtg |
| 36301 | atgagactca gcttgtactc aagaacagat ttatgactag gtgagaacta aaatttctct |
| 36361 | cagaaactaa ctaaaaactt tcatggacaa attgaatccc cttgggattt actatttatg |
| 36421 | gatatacgcc aatttaatta ttggactaaa taaaatactc tttattagtc cacttatgga |
| 36481 | agatgactgt gtaaacatag tatttgcaag taagtacttt ttggtattat atttatataa |
| 36541 | tcagctcttg tatccatgta gcttatagtt ggctgcttca ccaggactct acaaattgct |
| 36601 | cagaacccaa ggaggtaaga tttttatatt gcctgttttc agtatgtaaa agtccagcca |
| 36661 | caaaactatt aatttaccct tgactatatt tttacagttg ctgtggagct gaacctaaaa |
| 36721 | ttttgttaga tggttacttg gtttgttaca tttttatata ctaatttata tactataata |
| 36781 | atatactgta tatatttttt aaagtctggg gtttgttttt agaaagtata ttctatcttc |
| 36841 | aaagaattat ttggtagttt catgtgtgta tctggaaaat aactactgta cttctttata |
| 36901 | aaagtgagag tgaggtccct gctagttgag gctggaatct agacgtggct gtttatgtag |
| 36961 | gagttagtaa ttaaacagtg cctgtttgct gactgcctca tttttgccag gctctgtatg |
| 37021 | ggtgccttgc aaggcattgc ctcatttaaa ccttgcagca gcattcagag gaaagtatta |
| 37081 | atatgcccac tgtacagatg aggaagttga ggaatgcttt attagtaggt gtgaagtgtc |
| 37141 | ttaagcataa tacctccctc ctcaaccttg ctccctcatc ctagttaagg gaattgagac |
| 37201 | acagagataa ggcaatttgc tattgacctt cagcagatga cttaacctca ccgtggctta |
| 37261 | gtttcttcat gagtaaaaca gggtacctat cttttaggat tggtgtgaag attaaagaag |
| 37321 | ttaatacata tacaaaactt tagaacagtg cttggctagt agtaatctct ccataaatgt |
| 37381 | tagatgttat tattaagcct gtgactacct gttatctcat gtagttgtcc agagataaaa |
| 37441 | atgtaggcta taggaaagaa taaggaggaa agccaggctc ctaatctgcc taagatagaa |
| 37501 | gtgaatctta gttaagtttt aagataatcc tatgtgcttt taaaagattt catggtttgc |
| 37561 | ggtttgcttt tctttggctc ccagtttctt tttttttttt tttgacttct tagtacaggt |
| 37621 | aacttttgaa ggaattcagc ttaccttaga tgaaccaaag gaaagctaaa agccgagata |
| 37681 | atttagaaaa actaaagcag agattatgtt cctaaattcc ctctttctgc ttcaaagtcc |
| 37741 | aggaaacacc acctgcagat acagttgtat cccattgtta gaccctttcc tgaagccatc |
| 37801 | ctcttttctt tgtcttccca gagagaaact agtcattcga caaagattga acttcttctg |
| 37861 | tgtgttaggc tgtgctccaa gtgctgggaa gacaccagtg aataaaacaa aaagtccctg |
| 37921 | ctctcatgga gtttacagcc actattctaa agtttgtctg tattctgcct gtccttgttt |
| 37981 | ttatactttt agcaaatatg tgtatagaat tgctttgcag ccttgttcag catattgtct |
| 38041 | tcttttgttt tttaggaata tctgtttatg taattagatc tgtttttgat aaattgtatt |
| 38101 | ctgttattta tccttcctac cttctaatga gcagttaggt tatttccaac attgactatt |
| 38161 | atggttctcc aaaaagatca ttgaatatgt caccttggat ggatggatgg gagagtttct |
| 38221 | tcaagacagc atgtttcaga ctttttgttg tgaccacagt aagaattatg tgcacctcag |
| 38281 | cttagtaaat acaaatatac acttgggaca aaaacttcaa gaaacagtat ttacttactt |
| 38341 | ttatcacctg ggatgcactc tattttgttc tgttttgttt tcttgagacg ttgtttcgct |
| 38401 | cttgctgccc gggctagagt gcaatggcat gatcttggct cactgcaacc tccacctccc |
| 38461 | aggttcaaac aattctcctg cctcagcctc ctgagtagtt gggattacag gcgccagcca |
| 38521 | ccacacccgg ctaatttttt gtattaatag agacagggtt tcaccatgct ggccagtctg |
| 38581 | gtctcgaact cctgacctca attaatccac ccacctcagc ctcccaaagt gctgagatta |
| 38641 | caggcgtgag ccactgcgct cggcctattt tatctttttc aaatgcagct tacacacaac |
| 38701 | ccagctgatt gggttgtgtg taacccatac tgattaaact gatttatcac ccgtgttcat |
| 38761 | tatttttaaa acgttgtttt gagatctata attagaagtg gaattgctga gtagttgctt |
| 38821 | aagtgtatct tcaacttctt caactagctg ttgccagttg caattcagag tggttgaacc |
| 38881 | aatttaaact cctatcggca ctctagggga gttcctgttt ctccacattc tgaccaactc |
| 38941 | ttgatattgc cagatggatg agaaatggca ttcgttttaa atgtgccttc ccctatttac |
| 39001 | taatgaagtg tttaaagtgt cttttaaaat atatcacata gaagtttaat ttaatggtaa |
| 39061 | tcagagctgg cggcaaggag gaaatattat tgtgattgac tgcctcctct ggcagaaaaa |
| 39121 | atggtgtaaa aattatataa taatatctat attatattga gtatttataa tttgctagac |
| 39181 | actatacttt gtgcactgta tacattattt tatttaatcg tcataagaat cttgtaaggt |
| 39241 | tagtgccttc ttatatactt ttttttttga gacggagtct cgctctatcg cccaggctgg |
| 39301 | agtgcagtga tgtgatctca gcttggctca gtgcaacctc tgcctccagg ttcaagccat |
| 39361 | tcttctgcct cagcctccca agtagccggg attacgggca tgcgccacca tgcctggcta |
| 39421 | atttttgtat ttttagtaga gacggggttt caccatattg gccaggctgg tctagaactc |
| 39481 | ctgacctcat gatccaccca cctcggcctc ctaaagtgct gggattacag gcgtgagcca |
| 39541 | ccacacctgg ctgccttctt atatgcttat acaggaacag atggagaaag agattcggaa |
| 39601 | aggttcagta gtcacactat tactaagtag tagagccaga atttaaaccc acattggtct |
| 39661 | aatttcataa cttattttct ttagtacttc atgctattgg tgaattcgta gaggaaggaa |
| 39721 | gtttgtcatt actattcttt tctgttggtt tgtctcttaa atgtgtttaa ttttttgagt |
| 39781 | ttcactcacg tctctttttt tctttccttt tcccaaggta gtggagtctt tcctctgtaa |
| 39841 | aacttcacct tgttgtttct agtctaggat agtggtcttg taacctagga tagtggtctt |
| 39901 | ggttactgca attttggttg attatttctt gttggccctt aatgtgcccc catcttctgc |
| 39961 | cttaggcatt ccatctaatc ttttccagca gtttgcaacc ttactgtgcc tcagaatcac |
| 40021 | atatgaaaat ttaaaaatca tagataccta ggctccaacc ctaccctgga aattctgatt |
| 40081 | tggaaatatt tatttttcaa aaagtccaca gataattctg ttgcacagcc atggtagaga |
| 40141 | cccattttct taggggttaa tgatgcttta gagtcacgca gacttgaatc agagccctga |
| 40201 | ttcctttaaa cttattagtt gagtgatctt ggccaagttc tttaatcttg gctaaggttc |
| 40261 | ttcatctata aattggagat aatattatta tctaccttag agagttgttg aagagattaa |
| 40321 | atgaagtaat gcttgtaaaa cacttaccag agggacttag aatacttctt aaacattgcc |
| 40381 | tgctaataag gtgtcattat tacgttatcc tttatccaat caaaatactc acctttgcag |
| 40441 | tgtgtatcaa cttgatgtca gaaaaataat ctgataaata tcaccataag ggatggatga |
| 40501 | tagctagttt tcattactta cctttcgtta gttgaactgt actaggaggc agaggagtag |
| 40561 | gcagatggtt ttatcacgcc aatcagccag tcttttctgc ctatgtatca gtttggacct |
| 40621 | ataaggaaaa tgaaggggaa aacaaatgtt tttctttcct tcctaggagg attttcttct |
| 40681 | tgcccttttt ctcccagttt cctctctgag ctgagtatca ttagcatcat caatacccta |
| 40741 | gtttcagagc tgcgtagtga tttacctaga tacttttctc caccattatt ccctactacc |
| 40801 | taactcctct tcacctccat taaatctctg ttcatcatgc tatccttctt ttggattgcc |
| 40861 | tttagttcag ttgtgactta cttgattact tgaacccagg tgtctacatt tctaagctat |
| 40921 | tgttcaagta agggccaagg taaattctaa ccagctgtct ctttgccttg tgtgggccca |
| 40981 | ttctagacca gcgggtgtta acttttgagt attggaaaag gattagatta gctgagcctt |
| 41041 | caggcttgtc agcagggctg tcactagacc ctttgatctt ctttatatac ctaatggtca |
| 41101 | tagagtataa taataatggt acatgttatt actatatgat taatagatta ctagtagtaa |
| 41161 | ttttgttata tatgtataca gtacacttcg ttatgtatat atataattgc ttgcaacttg |
| 41221 | aaatttgagg ctacaacatt tacagtaatt taataactta actgacttta tcaaaaactg |
| 41281 | aaaaacctca tggaaaaggt agaatgtaag gtagacttaa aaggataggt atactttttt |
| 41341 | ctataaatag gatgaggcaa gagtttccag ttaaggaaac tgacatgagc aaaggcttga |
| 41401 | aggagcatgc aaatttggag cattgtgagt agattatttt ttagtttttc agagttcatg |
| 41461 | cagtgggaat aaaactaggg agtttggaat caagcttgaa aattcctgaa tgttcaacca |
| 41521 | agatatattt gagttagtag attttttggg tatgtcatga attcgtttgg cttttcttaa |
| 41581 | ttgatataca tacaacttag tcctgagctt acacatgttc attgtatcat taaacattta |
| 41641 | gcctggttgg tttaagttga tggccactgc caggtctcct ttctgccttc tcccctttat |
| 41701 | cccaatgctt tagttttttt ctgccttttt gtctgttgtg atctgtgctt catcaggctt |
| 41761 | tcccccagtt cctccttgtt aatctccaga ttccaagtca gcaacacaat gaatgtctgc |
| 41821 | ttatcgcgag ttgtgaattc ccgcatatct ctttcactta ttaaaaagat tgtttggtac |
| 41881 | atattttgtt tataagacag ttattgactt tcctgtttct cttcctttgc agacctagag |
| 41941 | gatcaagaca taatgggagc atttttagac aagccaaaga tggaaaagca taatgcccag |
| 42001 | gggcagggta atgggttgcg atatgggcta agcagcatgc aaggctggcg tgttgaaatg |
| 42061 | gaggatgcac atacggctgt gatcggtttg ccaagtggac ttgaatcgtg gtcattcttt |
| 42121 | gctgtgtatg atgggcatgc tggttctcag gtcgccaaat actgctgtga gcatttgtta |
| 42181 | gatcacatca ccaataacca ggattttaaa gggtctgcag gagcaccttc tgtggaaaat |
| 42241 | gtaaagaatg gaatcagaac aggttttctg gagattgatg aacacatgag agttatgtca |
| 42301 | gagaagaaac atggtgcaga tagaagtggg tcaacagctg taggtgtctt aatttctccc |
| 42361 | caacatactt atttcattaa ctgtggagac tcaagaggtt tactttgtag gaacaggaaa |
| 42421 | gttcatttct tcacacaaga tcacaaacca agtaatccgc tggagaaaga acgaattcag |
| 42481 | aatgcaggtg gctctgtaat gattcagcgt gtgaatggct ctctggctgt atcgagggcc |
| 42541 | cttggggatt ttgattacaa atgtgtccat ggaaaaggtc ctactgagca gcttgtctca |
| 42601 | ccagagcctg aagtccatga tattgaaaga tctgaagaag atgatcagct cattatcctt |
| 42661 | gcatgtgatg gtatctggga tgttatggga aatgaagagc tctgtgattt tgtaagatcc |
| 42721 | agacttgaag tcactgatga ccttgagaaa gtttgcaatg aagtagtcga cacctgtttg |
| 42781 | tataaggtag ctagactttt tttaaaaaca taaaatgatt ttatgccata ttaatcacta |
| 42841 | ctctagtatt taatcatctt agaatctgta attctgaaac cagtttttgg cacaactgta |
| 42901 | ggatactgtt caccaattat gaaaatatat cataggacca ctatgtagaa ataaattacc |
| 42961 | caattaccat ctctgcaaga gttataagct gaatgtttag tcttactact tgagctttgt |
| 43021 | aataatgtgg aagattatca aaggtaaaaa gattttatca gagtgcatgt tttgaaagaa |
| 43081 | agagggtggg gagggttagg cctcagtgtc tataaatgaa aagcatttta gagttttaat |
| 43141 | atttgaccat gtgatatctc agtacattta tgatatccta atcctttgaa actaaggttt |
| 43201 | aatggtctgt agcacttgaa agaggaatgt agtgatcatt agacgtgatg taatagaaat |
| 43261 | agccttgtac atggagtcag aagaacttgc tctgagactg tgtatggttt taggtaagct |
| 43321 | atttaaactc taatcagttt ttttcatcat aaaaaggata agacattatt atcttgtctc |
| 43381 | atggcgttgt caggaggatc aaatgggata atatacacag gtgagctagc tcagcccctt |
| 43441 | tattttacag atgaggaagc tgaagccaga gaaggttcta caaacttaag agtcagacag |
| 43501 | acctgagacc aaggcttacc tctattagtt ttgtactcta cattcctgaa cgaattgttc |
| 43561 | aacctctctc agctttaaaa tggagataat agtatcttat aagactgttg ccgggcgcgg |
| 43621 | tggctcacgc ctgtaatccc agcactttgg gaggccgagg tgggcggatc acgaggtcag |
| 43681 | gagatcgaga ccatcctggc taacacagtg aaaccccgtc tctactaaaa aacacaaaaa |
| 43741 | attagccggg tgtggtggcg ggcgcctgta gtcccagcta cgcgggaggc tgaggcagga |
| 43801 | gaatggcgtg aacccgggag gcggagcttg cagtgagccg agatcgcgcc actgcactcc |
| 43861 | agcctgggcg acagagcgag actccgtctc aaaaaaaaaa aaaaagactg ttgaggaatt |
| 43921 | aatgagcgta tgtatatata tatatatgtg tgtgtgtata tatatatata catatatata |
| 43981 | tatataatgc ctagtttagc atataataca tgctacaccc tttcctacag atattataaa |
| 44041 | tgacgtgtac agggttttat acattgctaa cagcattaag actagaaaat caaggccctt |
| 44101 | gacttgggtt cagtgttctt tctgccacac tctgttctgt tttgtcaata atgcttatta |
| 44161 | acttattctt tgatgttttt ttaaacaaat gttgactttt taaaattctt ctgagggtat |
| 44221 | atatttgata ctcaaatatt taattatagc atttgaatac ttagatgtct ttaaaaatga |
| 44281 | atgtagttcc taaaagttgt cagtttttaa aattaataca agttaaaagc tggtttataa |
| 44341 | accttaggta gttttacctc ttatttcata tttctactta tgaagcgaat gtattagccc |
| 44401 | cacaatttat aatttatttt aaaggtaatt ttatcaggta catataacta caggtcaaat |
| 44461 | cctattgtaa acaatgccac taaaattttc tgtcatgttg tatggaagga gtgtgtgtac |
| 44521 | acatgtatat ttgtattagg tatcacttca aattaagtca ccagtaaatg tttagaaata |
| 44581 | gtattgtttt atagaattat aaaaaattat tattacagtg agattaacct gtaatgtttc |
| 44641 | tctcctgtct tttggagtat tggttatact gtattctctc tatactgttc gggggagggg |
| 44701 | gagtcattgc acacacacac gcacgcatgc gtgcacatat gtattttttt ctccctgaaa |
| 44761 | gtaacatttt cactagaaca agtataactg taattggcac agctgtaaag gttctcaagg |
| 44821 | ctttgaagag caaaaagaaa actaaaatat tctcaaattt ttttcttccc agggaagtcg |
| 44881 | agacaacatg agtgtgattt tgatctgttt tccaaatgca cccaaagtat cgccagaagc |
| 44941 | agtgaagaag gaggcagagt tggacaagta cctggaatgc agagtagaag gtggatcatt |
| 45001 | taacaaaaaa taagtagctt tattaaagaa aaatcttcaa cacaatcaaa ctttaacatt |
| 45061 | ttagctttta gatttttatg tgtctttgac agctagtgtc tagtgtatga aagtggaaaa |
| 45121 | ggacatttct gtagtagctg ttaagatgta tgcagttgtc ctatcacagt tatattagaa |
| 45181 | ataacaacta gtgtggaaag taacccacat tctgttcagg atacttttgt tttaaaaggc |
| 45241 | taaaatgttt gtgtgcttta aaattcttaa tgaattcact ggcatgagta attttatgcc |
| 45301 | gggaaaatct gatttgtggt ctatttaatt acaaagagat tgtgatggtg ataatttgaa |
| 45361 | taccatcttt ctatgaaaag ctgtgtatat taattttaat ctctagggcc ttttttcaat |
| 45421 | aacttgaaaa tcacagcctc atcattagca actttatata cacagcttta aaaaatttgc |
| 45481 | tttaaactgt ttcagagctt cttccctaag ccataaactg tctcacctaa acaagataga |
| 45541 | cctcaataaa agacataata tttctccacc tggcagaaaa gtaactgcca agtaagatta |
| 45601 | agaaataagt tgagcatttc tggagtatat ttctcataat tctgcagaga cttccagttt |
| 45661 | attgcatata tgcagtaaag tgtatatatt atgcagtttc tctaagtgga aaaacttgac |
| 45721 | atgactttaa tagtgttaaa acatgaaaat gctggtgagc tgaggtagaa aaaaaacaag |
| 45781 | aaaaaaattt taaaaatagc gttaaaatgc attttaggct cctaatgtcg ttaatttgca |
| 45841 | cgttaaagac ctctaatcgc atgcaagcat tgacctacca cacctacacc ccactgtgcc |
| 45901 | tgcattattg aaattactac tcactttaat tttttttccc ctttttactg tttgatacca |
| 45961 | atcataatca gtctgatact agaatcatga ttggttttgt tctttagtac tgtctagagc |
| 46021 | tactattttg tttttattgc tgtccgccac ttgatagtca tacatctagt atgtgtttgt |
| 46081 | actatttagt attgcattca ttggacgtac tattcaacga atttgtctcc ttactgttac |
| 46141 | caagggtctt ggaattttta tattttaaat taatcattca atataagtta tttataggta |
| 46201 | tagtacaatt ttaaaatatt tagaatgcta caatcattgg ttcaaactat tgattcatgg |
| 46261 | gaaaatttaa attcataaga ataatatgta tagggataag ttgatattta atgtattggc |
| 46321 | actttaactt gggcagacat gttttcttat tggtttaaag aagtaataag tattttaata |
| 46381 | tttttactat gaaatgggat ccaattttag aaatgaattg ctacttttct ctttagtgtg |
| 46441 | tacactttaa gttttagatg gaattatgaa atattcactt tacgttattt tgttacaata |
| 46501 | ataagtttta tatttcttat tatcaagcaa gagttgccac ctggacagag tttacattga |
| 46561 | tactcaatat ttttgatatg ttaaagtgtt attatacttg taccattcaa ttggagattg |
| 46621 | tcataaatta aatatggtat tttttactca gatttgatga gactgcaact agagaataaa |
| 46681 | tattacatta aaattgctgc tattttaaaa catagaatgt aactgtttgt attgtaaaag |
| 46741 | ttgtgtgtcc tcagtgaaaa atactgcctt cagcttccaa agcaggcacc tgaatgcctt |
| 46801 | tttcttttct ctctctcttc tttctttaaa tacatgcctt cactagattg ctgtgaccta |
| 46861 | ctaaacctca ttttatttgc ttggaacatt tttggcttct ctgtccacac catagaaaag |
| 46921 | gacaaaggcc ttccagctct ccttttctct tatttgttcc cttctaaata cttgactctc |
| 46981 | tgggggtgtg aaaagattca gctcataatt tatactacta ttaattttgt ggtccttcta |
| 47041 | agccaattta aaattatact tttgtgtata tattttcaaa atctttgcta ctgactacct |
| 47101 | tagctttaaa gttgctaatt tggagatgac tgatagaatc cctgaaactt ggtggagggg |
| 47161 | agtggaaagg agcagaatgt gtttgtgtat atgtatgtga aacatggatc ttctatatat |
| 47221 | aaatattctt tagaaaaagc gttcagtctt tttacaattc taagtaaaac gtataggtca |
| 47281 | acagaaatta aaggaatatt tcttaaaatt catttatttt tcttttttga aagcagacag |
| 47341 | aagcaaggct agtatagtat gaatggtcag gtagtttaaa tgcgttgtcc cacgtggaac |
| 47401 | aggagatatt aactgttttt aaagctttac attatttttt tacaagaatt tgttttcctg |
| 47461 | taattttctt cacagttaaa atatcctaat ttatatttta acaaaagaga tgtaagatac |
| 47521 | atcaaatata gatgattcaa ggtaatgttt tttagacctg aaactattga ttcaatttat |
| 47581 | catcagtgtt tatttctatt gtatctagtt atctatccaa ataattgtat tggcttagcg |
| 47641 | gagactgtat gttaagtgta ttagttttag attgtgaaat tgtgcagttt aggtaaacat |
| 47701 | caaatccctt tatttttgta cagcagattt ccagcattag aaccatgtag aacaaatata |
| 47761 | ctagatgtta aagattacac aataaaggaa cttggcaaac caaatctgag atccttgctt |
| 47821 | tttgttcaga tttaagactg tttttttttt taaggcaaaa aataatagat ggtaaaaatt |
| 47881 | tggcctttat tctaaaagga cagtttgaac catttaaaga aatattatgg gtagccatgt |
| 47941 | ctttaaaaat tttgaggcct gatcaaagga cgaatcttta tacacattgt tattttcaaa |
| 48001 | ggtatttaca aattgagaag cagaatgatg atttcttgcc ccactaatga caaataaaat |
| 48061 | accatttctt accataaaca ttgaaaaaca aactgaacaa aacttgacca tataacttac |
| 48121 | aaatactttt gacttgaatt tgcatgtgga gtatgtgtgc taaaactgca agtgaagact |
| 48181 | ttttatactt agaggaatag ttgtagagtt ctctgtaagg ttctttgttc ataatttttt |
| 48241 | atattttgtg atgtgaataa tagtatgcat ctgcatgaca acactgcaga tcatattgct |
| 48301 | gtgccctttt aataactgga tattttaaaa ttattttaaa tgcagttgta tggtttatca |
| 48361 | aatggttctc tttttaaaca ttttataagg aaatttagac cttttctatt caaggctttg |
| 48421 | ttgaagtaat cagtgaagga aatggtatta tatatctata cagtatattg tttaaaggta |
| 48481 | ctttagagaa gaattaaact gagacagtga tgttcaagta cataatctgt gcatgctatt |
| 48541 | taagtatcat tttgcaaata taaatatata cataaaaata attgagttag tatataatct |
| 48601 | ataaattccc ctataattaa gggttgttat tttcaaatct tgaaattgtt tattttttaa |
| 48661 | aattccacgc agtgtattat atagggcaaa gttcaggaaa catgtttcca gtctaaagag |
| 48721 | aggagtgatg taagaagaaa ctagttgaca tcttaagaaa gggtattctc acttgtgcac |
| 48781 | acatatcctt gattatataa tatgctgatt taacatgaaa tattttttca cattttaatt |
| 48841 | ttgatttgaa gttaatctta tatgtcattt tgtgcatttt ttgtcataaa tcttcaaagg |
| 48901 | tcaacctgat tttttttttt tttaaatgat accagattag aaaaatctca gatgtggtaa |
| 48961 | atgccatctt taaaaagcta ggttatcttt gtttcggttt tcttttcaat taaatttgga |
| 49021 | taacacttac aaaaaaagta cttctgattc ccagaaatca taaagaagca gggggaaggc |
| 49081 | gtccccgact tagtccatgt gatgcgcaca ttagcgagtg agaacatccc cagcctccca |
| 49141 | ccagggggtg aattggcaag caagtaagtt acattctgta cactcttatg ctttatgtca |
| 49201 | gtgtatgaaa atgttaggta ttcattgata aaatgtttgt ttgcctaatt tctgaactga |
| 49261 | tgcagttatc tgtgttttaa cctgtgattt caattttttt ttagtattag aatatctaga |
| 49321 | gtttgtttag acataggtat cagtatcatt gtaactaatt gcagtgaggg ttgtctttat |
| 49381 | tggaagcttg gttggcattg atttcctcat agagcataaa cgagataata tactgaaaga |
| 49441 | actttttaaa ctaaattaca tagcactaca taaaaattta caaaattcta agtgtcgttg |
| 49501 | ttagtagtag tataatttga tttggtcaat ccctgagtaa cttaacaccc tgaaaattcc |
| 49561 | ccttcctcat atggaccatg tttctatgcc tgattttccc agtatttttg gttgttgatt |
| 49621 | gctggttagt tggttttagg tgtcagggaa gaagatagaa tatatttctt gtcttttccc |
| 49681 | tttccacttt agttcataga attggactac tttgtgttaa atattaagct tacaccttta |
| 49741 | cataataaag tggaaaattc aagcaattcc aagggttaaa acatttttac ctcaaatatc |
| 49801 | catataattg acttacaaac ataaaacttg caaatacctt tagcttgaat ttggatatag |
| 49861 | agtaaatgtg ctaaaactac aagtgaagac ttcttgtact tacagtgaag atatattatt |
| 49921 | gactaatttt ctcgattctc tactgtgttt tgcaagtgtg accatgttct ttgtaatcac |
| 49981 | taaatagtga ttgtaaaagc tcctgtttga agcttttatt ttttggaaac tttttttttc |
| 50041 | catatgactt tctatgaaac gatctataaa attgcttcaa gtcaatttcg gaagtaggga |
| 50101 | gactataact tataaatata ctacagattt ttaataaagt gttactcaaa actaattcct |
| 50161 | agacttgtca tggaaatgtc ttatttgaca ttccagtttt aatagaacct ttgaaaaggg |
| 50221 | cattaagaaa agtagaagct taaatataaa gtataatctg agacttcaaa actacgatgc |
| 50281 | aactcaacta gcttatgtta gtattatatc ttaagaatgg cattgccatt gatgactcat |
| 50341 | aaccatttaa accaattcaa atggaagtag actatataga cttttttttt aatctgtaat |
| 50401 | ttttagcctt ctattaactg ggttggtaga gtgttataac acattttaga gtagactcac |
| 50461 | ctctgcttta acataggagc ccaaatatct taaaagtgag gctcactatt gacagcaaga |
| 50521 | aatagaacta aaataatatc tgagtataag attatcttag aaattttaat aaacacctgg |
| 50581 | ctatgagtta ctaagcaatg ttttgctttc tgaagaatta attttctgca tttttttaca |
| 50641 | cttaggagga atgttattga agccgtttac aatagactga atccttacaa aaatgacgac |
| 50701 | actgtaagta gcattttagc tccctctgct ttcccttccc ctccactcta aatgcatatc |
| 50761 | cttcctacct gtttttgcag agctgttcac tgtacccatt ctcttacaca cacccctgat |
| 50821 | tgattgctct gaactctgct tgctctgatc tctgcctgca ccacagttct agtgattgag |
| 50881 | gacataatca tggtgagtgg ccacctctct caaaaaagaa gaaagtcctt ttgaattatt |
| 50941 | ctagattttt taagtcttta caaacaatca acttcagaag gtttgcctcc actacatggt |
| 51001 | tttttttttt tttttttccc gaagttggga ctatctctac tcagaatact gtcttattct |
| 51061 | gagttttgta agcagctgca gcagaacttc ggaatgtctg tctcaactaa atagttctta |
| 51121 | tggttggaaa tacctttgtc aggaatcctg aagtatatac ttcacttaaa tattagagaa |
| 51181 | ttgtttgcat aaatagacat tagaatgcca tactctccag ttaagtgttc tgttgcaatt |
| 51241 | ctgttgtatg atgtttatag aaaagtgata acttaaagat catgtgggca cagctctgaa |
| 51301 | atattttatg tattgcatta tgaagcttcc tcacatattg agttcttaat cttttctgat |
| 51361 | gcattaaagg tttataaaag tggtattcta aaactgtatt ggatttcatt aaaaaattct |
| 51421 | ttttagtaga tttattgatt gagtaataca tttggacagc ctcatcataa aaccgcattt |
| 51481 | caaatatttt ccaaaacaaa acaaaggatt aacaattctt tgaacagatt aaacattgct |
| 51541 | atttcatatg tacacatata tacttatata ctttaaaaaa catttatatt ctaaaagtca |
| 51601 | tctttacaga acattatctt tctccattat ccagaaagta tggtgtattt tggcaccgct |
| 51661 | aaattttaac gttgttcctt attttgcttc cttttacaaa ggactctaca tcaacagatg |
| 51721 | atatgtggta aaactgctca tctagccatg gagtttacct tcacctccaa aggagagtac |
| 51781 | agctcaactt tgttgaaact tttaacatcc atcctcaact ttaaggaagg ggatatgaca |
| 51841 | tgggtgagaa tgattacatc agagaacttc agcagtacaa cagctagccc agaactgatt |
| 51901 | tttttttttt tttttgtaaa tttgagactt atgtaagcgt gatttcaaac cataattcgt |
| 51961 | gttgtaaatc agactccagc aatttttgtt gtatgatttt gtttttttgt aaagtgtaat |
| 52021 | tgtccttgta caaaatgctc atatttaatt atgaactgct ttaaatcact atcaaagtta |
| 52081 | caagaaatgt ttggcttatt gtgtgatgca acagatatat agccctttca agtcatgttg |
| 52141 | tgtttggact tggggttgga acagggagag cagcagccat gtcagctaca cgctcaaatg |
| 52201 | tgcagatgat tatggaaaat aacctcaaaa tcttacaaag ctgaacatcc aaggagttat |
| 52261 | tgaaaactat cttaaatgtt cttggtaggg gagttggcat tgttgataaa gccagtccct |
| 52321 | tcatttaact gtctttcagg atgttccttc gttgtttcca tgagtattgc aggtaataat |
| 52381 | acagtgtatt cataagaatc tcaatcttgg ggctaaatgc cttgtttctt tgcacctctt |
| 52441 | ttcaagtcct tacatttaat tactaattga taagcagcag cttcctacat atagtaggaa |
| 52501 | actgccacat ttttgctatc atgattggct gggcctgctg ctgttcctag taagatattc |
| 52561 | tgaattccat tttatcaata aagcttgatt taacaaacaa gaaacttaat catgtatgtg |
| 52621 | taattcctct tttaccctgg ccttttaaaa cactgtgccg ttgtaatgag acgtttctca |
| 52681 | tagggaaaga tgttagtctc ttttaattgg acaacactgt cactcaaggc atagatgaaa |
| 52741 | ctttccttcc attagaaaga ctaaaagatt taattcttgg ttgtacctta atctattttt |
| 52801 | taaataggtt tctttcaggc tgcttatttt tcattaagat gtgtatcagc ttggatttgc |
| 52861 | ctactgttta attaaaatat ttattgtcaa agcttgacaa tctaacactc tatggtaggg |
| 52921 | gtgtgtgtgt gtgtgtttgt gagtgtgtgt ttgcctgtga tttttaattg gcccatgtct |
| 52981 | ttagaatcca agtggttaag atgtatttgt gatttgaaat atagcatgtt gataatattt |
| 53041 | agctgttggc ctttacaaat aactttcaaa gcttaaggaa ttgtagatat aaaaataacc |
| 53101 | taatttaatt taggcttaaa ttcctctgat taagcatgtg aaagtaagtt ttaaaatctg |
| 53161 | tcgcattgaa aagattactg ttccgtgccc ttctgtattt ttgtctctct aggttgaata |
| 53221 | ttgtatttat caccatgtaa tcattcagta ggcagattcc cactagaaaa ctgttgaaat |
| 53281 | gtaagactaa aatacaacat tgaatacaaa atcaaaattt tgtgtataaa aaccagtata |
| 53341 | gtccattttg ttatatttgt tttttcccta acctggaaat atacatattt gtatatatag |
| 53401 | ccttaaaatt aatgtaaagt taggggagta gtggggaaag taatgtgaaa tgtctcagat |
| 53461 | ttaagtagtt aaataccagc aaaatctttt cattatccct cttattttgt gaggtgatta |
| 53521 | aatgtaactt aattgtattt aatttatatc ttattccagc atgaatgagg aaaaactgaa |
| 53581 | gtactattta tatttagaaa ttcatatcag ttgaaattac agaaccaatt ccatacttac |
| 53641 | aataaatact taatgtctaa atctgtggta gagtgcgaag tatgataatg ttctaagtta |
| 53701 | tggctttgca agcatctaaa tgtgcattta atgaatacca gtgcttctag tatagactaa |
| 53761 | ttaccagaca tactggtact gaaagctaaa tccctattat aacaaaccag ttccttaata |
| 53821 | ttttaagtag actgacaact ttagttccag aaattgcaaa actttgaact ggactgtgta |
| 53881 | atcttttgag atgcaaaact taagtcacaa gtagagtatg tgatggaaag ctgtatttca |
| 53941 | aaccataaca gcatatttag agcctttttt ttttgagtct ttaaacaaga gaaaattaaa |
| 54001 | atattcctgt caaaattatt agtattgaaa ttaggcttgg acacgagaga gaaccgtatt |
| 54061 | tgagtgatgt gagaagacta aatcttttcc acatgagtca gcactgccat actaataatt |
| 54121 | tttttactat aaaaatacag gaaggaagta tacattataa cagcagactg tgtgtgttcc |
| 54181 | tgattcctgg aggtaatagt ggggggaaac caaccatact ttttaaaggc acttttgcac |
| 54241 | ctctattgtg cacttcattc ttgtaccact taaattcttc acccccatcc cctttttttg |
| 54301 | tgctaattag catctcaggg caatgcctca aaaatgtttg atgtgttctg ttctttggag |
| 54361 | ggaaaaagtt cttatgtgat gataatatag tactcaaaat atacttttat catttaaatg |
| 54421 | tcttatttgc tgctatgaat aggaaataac attttgtata gcaggctctg ttttacccta |
| 54481 | acattaaaaa atttcactga tctttctttc attaacaggg tagaatctcc taatttccac |
| 54541 | tttcttggga atatactttt atagacaata gaagcagttc tcaatattag catatacttt |
| 54601 | aaaaaatcaa agtgataact taattcagct ttggaagtat ctcaaacata tttttacttt |
| 54661 | atagtgcatt aacttgcttc tagagtactt aatgcaactg ctctagccac ttaatttttt |
| 54721 | atactaatct caacattaag aaatttggat taagtaataa attagttatg taattcaagt |
| 54781 | aatctgaatt acagcagtac ttttagtgat cattcatagg actatatact aacccagcta |
| 54841 | ataactcagt ttttttacaa aatgtttcga gtattattgg taaaacactg ttctaggcta |
| 54901 | agcacattgg gactgtaaag aaatgagtag atccttggct tcaagtttac atctggacaa |
| 54961 | tttataatct agtgtatgtt agtattataa ctggatcact catcaaaaaa tatatatata |
| 55021 | tatctattgc ccacctgcta tctaccaggt acttagctga tcaaggcagg cccctgccct |
| 55081 | aaagaccttg tttatacttc ctttactcac ctgaaaactg ttctccagtt tattttcttc |
| 55141 | tctctaaagt taaagagtaa ttcagaagaa aattttgctt agcataagaa taaaattgga |
| 55201 | ctgaagaggc ttaagcccat tcagtatcct tgattgcatt tatccaacgg cctttattct |
| 55261 | tcctgctgac agcagtaact cagaggaata ggtagtagat ttctgaaaat tatccagcca |
| 55321 | tggaaatgta ggtggggttt gagtttaagg catttaaaaa tgtaaatatc tctagctaaa |
| 55381 | tttatcttaa gtagaactct gtgtttttgt aacacactgc cagtgttaat atcaaatttt |
| 55441 | agccaaatta ttactatgtg ttttaatatt ttaaaataat ttcactgccc atctttactg |
| 55501 | gacaaactca tttggagttc aacttgtgat ttctgaaaga actgatgaaa ttgggtactg |
| 55561 | ctttttttct ccatttttcg ttttgtttta atcttgaatt tcatggtata tacttttagt |
| 55621 | tcaaactcag ctgtttgtac agtattgtat taggatttgg tattagaaaa gatgtgtaaa |
| 55681 | tatcttagta tataattgtt tctcatttga ggtttttctt ctaagggacc ttaaagagtt |
| 55741 | ttatatactt ttgctcacag aaactgctgg tgagattacc attttttgag tatctagtct |
| 55801 | tctagttttt cttttaggca ttaggaagcc ttctttagag ttcaaaatct tagaagccta |
| 55861 | atttgctctt acttccttca attatgtgcc atgtgttttg gtttgtatat gttttaaatt |
| 55921 | gtatatttcc ttggaatatg cttgaaatat ttaagaatac attttcaaaa tgtataatac |
| 55981 | tgtattgttt tgttgatcag aataataagt ctcagttaaa tgtttgttat tactgatagt |
| 56041 | caaaatgctc aatagaaatg atgagaggca ttggttccaa ttcattgtca aatgaacgtt |
| 56101 | ttctaatttt gttcacagat tctttccctt tcgattgttc tgtatgttaa gatagtggct |
| 56161 | tctgctctca ctgttttcct atttatatta ctagcaggta ggagtgctaa ttagaaaaac |
| 56221 | ttagatggta ttgaaattac agttgacaac ttatattttt atgagatgga gaaaaaagat |
| 56281 | taagttgata taacaacaaa gtggactttt tttcttcctt atcctgcacg aaatattgcc |
| 56341 | cttgtttcct ctactttcct cttggtgttt tctctttttt tcaaacagaa acaggccaat |
| 56401 | tccattttct tgagcaagaa agcttagtgt gttacttcat caaggccagc taatactgtg |
| 56461 | ttaaaccggg ctgaaaatga gaaaacttgg gagatggagg aatggggaaa tggcagtggg |
| 56521 | ataggtaggg aaggattact cttaattgtt ttaaaagcca taggaaagtc ttccttgtac |
| 56581 | gtggctgtaa atttataaga actattgtgt cacataaacc aacaagaatg aacctttgct |
| 56641 | gcttcagata atttgatttt tccagcaagg aaattaataa gttactgatt cttcagcata |
| 56701 | gaaacaactg agaagaatta atgcaatgtt tcttcactag aaaacccaac ccttcatttc |
| 56761 | ttttcattgc tccaaaaccc agttttcaac taatggtttt ctcattaaac taaatgttta |
| 56821 | gaaaagttgt ttagagtttt tctttttctt ttacatagtc ctcctgatcc agtataagac |
| 56881 | tatttagtaa cgtgcatttg tatggtacta tctaaagtaa gttagattga tgtaagagat |
| 56941 | cgggtagctg cggaacaaaa ttagttatat cctaattagg tacagtgaat gacacaaaat |
| 57001 | cattttagca atgcttctta accttttggg gtcacaggcg ttttgagact gatgaatcct |
| 57061 | agggacttat ttacccagga aaatgcgtat ataacataca tatctcccta aagtttacaa |
| 57121 | tattgtagtg gttcatgggc cccctggtta agagcccatt ctaaagtaca atagggcatc |
| 57181 | atcccttttc ctgcaaagcc caaaagtata tttctagggc atgaaaataa cttgagtcta |
| 57241 | ttttaaggaa ttgtttcact ctagaggtag ataggggacc tggctagaat ctgacattaa |
| 57301 | aatatacttt ttaaaaaata ttatatttgg ggtggggaaa gtgattaaaa ggtgaaaaaa |
| 57361 | aaacatagta ttcagaagtt ttggaggtta atgtctttct ctaagatttg ccactttaga |
| 57421 | aattcaacag aaaagaggta aaacagaaat ggaatgtatc tggaacattt ttggcctcca |
| 57481 | tagtgcagat atactatatt aacaagtaat acatttattt acctgtcaga tctccaggtt |
| 57541 | ttaagatttt gagctttcta gtattaggat tcattaaatg ttcaattcat ttcatattct |
| 57601 | aaggaattag gttatttact tactaattca ggatgttaaa ataacatcca agtcggacaa |
| 57661 | ccaccaccaa tgcacacagt taatgagatt tctaaaatat aataagtaca atgtaacaaa |
| 57721 | cgtatagaat tttgcatttg ttgccaaaat tagatgttta atgacagctt atttaattcc |
| 57781 | catttgtggg acttctggaa catagaaacc attatcttac ctggttatcc cttgactaaa |
| 57841 | tagcatatct gcaggaaaat atcttgtttg tagtgatatg ccccaatagt gattgatttc |
| 57901 | actcttgaaa tgagttatat cacttaattt gtataaatgt tatgagtgga gagacatgta |
| 57961 | catgttaaaa gcatgttgca ttatatattc attttttaaa ctctataaat gttaagaata |
| 58021 | atataattgc agaaatattt ttcttaaata caatgtgtaa caaaattctc cgtagcaact |
| 58081 | cacccacttt gcagtttaty tgatccacac ttctaaagaa attccataaa tgtatatttt |
| 58141 | gtattatgta ttatttcctg gtccaaagaa aatatgtgaa ttcagttcta actttaagaa |
| 58201 | tgtactgttt gttttcaagt tcattgaaaa attgcattca gcctgcgaat ggttgcagat |
| 58261 | tgtatgttag atgaaaagta gaaataattt ctagtttgga aaactggtgc cactaaataa |
| 58321 | acaggcaatt acataatgtg tgtgtattta tgattgttta atattgcaag cagcattttt |
| 58381 | attccaagaa acgtggagaa tttgaaatta gctgttggaa gaccttaaaa gaattgtttt |
| 58441 | tttcagctga gaaataaatc tgataggatt tgaactagat ttgttccttc ctaattttag |
| 58501 | gaagtttact gaaagccaag aagtttgagt tgatggttat tgtcatgaaa cattccagaa |
| 58561 | ataaagaagc cagagtagtt tctctttggt aagaacattc ccatttgtct gtcttctagt |
| 58621 | agatgattac tcccttgaag agttgaaatt gggaattggg agtggtctgg gaaaggactt |
| 58681 | cctcacctgg tatctgtggt atagtgcctg cttcaaaatg gcacttaaat gtgtgtgttt |
| 58741 | ctaggatgca gaaaggaggt gtttatttcc tctgccaatt ctgatcattg gctatattat |
| 58801 | atcctgtgtt cattcatgtc tgaaagtgac ttaaacaggt ttaattgatt ctagtcatgg |
| 58861 | cttccataat tagagaattt ttagagttga actgttaata aaagtgaaat tctgtgagcc |
| 58921 | gcaagagctc attttgttta ttgtaaaatg tgtaataaaa cagtaatgaa gcttcataac |
| 58981 | caaaggtaaa atatcttaga actgagtaag gtaaagtaag gcaaataagg ttttatttta |
| 59041 | tttatatgta agtggaacct cttcactcat caaattgtat catgcagttt tgttcatggg |
| 59101 | tttttttggg tttgggttcc ccctccaccc cgctttggag cttttcttct gaatgttgaa |
| 59161 | gctactttaa gttgttttat ttcttttttc ttaatttatt actgagaaac ttacattgga |
| 59221 | gtgcccttct ctttttcctt ttttatcaat taatagttca aattaaattt cccatcactt |
| 59281 | aatagctcag attaaacttc tcatcaagtc aaattttatt ctagttgaaa agtgctatat |
| 59341 | tttttgataa attaccctgg gaaattggtt tgcatgccct ctatagaact ttgcttactt |
| 59401 | gccacaaaac caacaactta ccagatgttg gtaaaagatt ttgctctgat ggctgcctgt |
| 59461 | tgtttacatg agcattatat gtatttcttc tgatatgcta tctaaggtga ttcagagtgc |
| 59521 | ctaaatagtg aattagctaa acattcaaca tgcttctgtg atagtttaga tacaggcttg |
| 59581 | tgtcttccag accaaagcac ttctctatag ctgtacaaaa gtttttctgg aaatcaattt |
| 59641 | gaactagcaa aatttacaat atttagattt ttgatctaca aaaatggcag ttttatctag |
| 59701 | cttaatttgt ttctgaaatt agttgttatt cttgaatgat cagattgcaa gtcagtattt |
| 59761 | ggtatttctc tcattattta tattttaagt cttttaagaa ctggatgaga ggtttaaaga |
| 59821 | aaagctatac atatttggac aaatttccaa atgtgaggct ggataatata caaatgtata |
| 59881 | cagatgctct agcattaggt caacatcagt atgagaaatg ttattccagc agtgactggg |
| 59941 | aaaggaaagg atgccgaatt taagtgttca gggaggaata taatagagac taaagatggg |
| 60001 | ggaaatgatt ttaaggattt attttgaatt acataattga ggtgagtaga gagctccttt |
| 60061 | ctaatatttt attgggatgt tgacgtggat ccaatttagt ctgtgactta gagacatcag |
| 60121 | tgagtaattc tctgaaaaca attgttcttt aaagtgcagt ctttgttaaa ggtggctgag |
| 60181 | ggctctaact ttgttctagg tgtcaactca aactctgact taggctacgg ccatgctgca |
| 60241 | ccttttctaa atatattttt tgtattttat tttactattt gtgtctgcct tggtaacagc |
| 60301 | taaaaattac ctagtatttt aaaggcaaag catttg |

In some embodiments of the disclosure, the PPM1A gene product is a PPM1A mRNA or PPM1A pre-mRNA comprising sequences from one or more of nucleotides 8470-8926, 41933-42787, 44874-45990, 49055-49164, 50647-50704, and 51703-58336 of a PPM1A gene sequence (for example the PPM1A gene sequence of NCBI Reference Sequence NG_029698.1 (SEQ ID NO: 1909). In some embodiments of the disclosure, the PPM1A gene product is a PPM1A mRNA or PPM1A pre-mRNA comprising sequences from the coding region of a PPM1A gene sequence, such as a coding region including nucleotides 8470-8926, 41933-42787, 44874-45990, 49055-49164, 50647-50704, and 51703-58336 of a PPM1A gene sequence (for example the PPM1A gene sequence of NCBI Reference Sequence NG_029698.1 (SEQ ID NO: 1909). In various embodiments, the PPM1A mRNA is PPM1A mRNA transcript variant 1, corresponding to NCBI Reference Sequence NM_021003.5 (SEQ ID NO: 1910).

In some embodiments of the disclosure, the PPM1A gene product is a PPM1A mRNA or PPM1A pre-mRNA comprising sequences from one or more of nucleotides 8470-8926, 9629-9730, 41933-42787, and 44874-47804 of a PPM1A gene sequence (for example the PPM1A gene sequence of NCBI Reference Sequence NG_029698.1 (SEQ ID NO: 1909). In some embodiments of the disclosure, the PPM1A gene product is a PPM1A mRNA or PPM1A pre-mRNA comprising sequences from the coding region of a PPM1A gene sequence, such as a coding region including one or more of nucleotides 8470-8926, 9629-9730, 41933-42787, and 44874-47804 of a PPM1A gene sequence (for example the PPM1A gene sequence of NCBI Reference Sequence NG_029698.1 (SEQ ID NO: 1909). In various embodiments, the PPM1A mRNA is PPM1A mRNA transcript variant 2, corresponding to NCBI Reference Sequence NM_177951.3 (SEQ ID NO: 1911).

In some embodiments of the disclosure, the PPM1A gene product is a PPM1A mRNA or PPM1A pre-mRNA comprising sequences from one or more of nucleotides 4999-5295, 41933-42787, 44874-44990, 49055-49164, 50647-50704, 51703-58336 of a PPM1A gene sequence (for example the PPM1A gene sequence of NCBI Reference Sequence NG_029698.1 (SEQ ID NO: 1909). In some embodiments of the disclosure, the PPM1A gene product is a PPM1A mRNA or PPM1A pre-mRNA comprising sequences from one or more coding regions of a PPM1A gene sequence, such as a coding region including nucleotides 4999-5295, 41933-42787, 44874-44990, 49055-49164, 50647-50704, 51703-58336 of a PPM1A gene sequence (for example the PPM1A gene sequence of NCBI Reference Sequence NG_029698.1 (SEQ ID NO: 1909). In various embodiments, the PPM1A mRNA is PPM1A mRNA transcript variant 3, corresponding to NCBI Reference Sequence NM_177952.3 (SEQ ID NO: 1912).

In some embodiments of the disclosure, the PPM1A gene product comprises a sequence including nucleotides 457-1429 of PPM1A mRNA transcript variant 1 (i.e., nucleotides 457-1429 of, for example, PPM1A mRNA transcript variant 1, corresponding to NCBI Reference Sequence NM_021003.5 (SEQ ID NO: 1910)), or a portion thereof. In some embodiments of the disclosure, the PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with nucleotides 457-1429 of PPM1A mRNA transcript variant 1 (i.e., nucleotides 457-1429 of, for example, PPM1A mRNA transcript variant 1, corresponding to NCBI Reference Sequence NM_021003.5 (SEQ ID NO: 1910)), or a portion thereof.

In some embodiments described herein, a PPM1A gene product is a PPM1A mRNA isoform transcript (for example, PPM1A mRNA transcript variant 1, corresponding to NCBI Reference Sequence NM_021003.5 (SEQ ID NO: 1910)), or a portion thereof. In some embodiments described herein, a PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA isoform transcript (for example, PPM1A mRNA transcript variant 1, corresponding to NCBI Reference Sequence NM_021003.5 (SEQ ID NO: 1910)), or a portion thereof. PPM1A mRNA transcript variant 1, corresponding to NCBI Reference Sequence NM_021003.5 (SEQ ID NO: 1910)

| ((SEQ ID NO: 1910) | |
|---|---|
| 1 | agaggcggcg gcggcggcgg tggcggcgct agggacggga gcgcgcgcgg gagctagaga |
| 61 | gcagtggtct cggcgctcgt ccggcccgca gcttcgggtc ctcaggcggc tgttgctccg |
| 121 | gaacgggtgg ttggggaggg gggggtgggg ggactctaga cagctgaggc gcgaaagcga |
| 181 | tgagtcctcg gctcttcctc ctccttctcc gggacccgct ctctgcctcc ctctccaacg |
| 241 | cccggatgat ctgagccgcg agggcgccga cagccggggg cccggacgca gcccggctcc |
| 301 | tcccctcctc cgccccttcc ccagcctgac ctggcccgcc gctgcagcgg tgacccctcc |
| 361 | cccggctgcc gccgtcgccg ccgcggtgac cccctccccg gctgccgccg ccgccgcctc |
| 421 | ggccgaccag ggacctgccc gcctgcggct gctccggacc tagaggatca agacataatg |
| 481 | ggagcatttt tagacaagcc aaagatggaa aagcataatg cccaggggca gggtaatggg |
| 541 | ttgcgatatg ggctaagcag catgcaaggc tggcgtgttg aaatggagga tgcacatacg |
| 601 | gctgtgatcg gtttgccaag tggacttgaa tcgtggtcat tctttgctgt gtatgatggg |
| 661 | catgctggtt ctcaggttgc caaatactgc tgtgagcatt tgttagatca catcaccaat |
| 721 | aaccaggatt ttaaagggtc tgcaggagca ccttctgtgg aaaatgtaaa gaatggaatc |
| 781 | agaacaggtt ttctggagat tgatgaacac atgagagtta tgtcagagaa gaaacatggt |
| 841 | gcagatagaa gtgggtcaac agctgtaggt gtcttaattt ctccccaaca tacttatttc |
| 901 | attaactgtg gagactcaag aggtttactt tgtaggaaca ggaaagttca tttcttcaca |
| 961 | caagatcaca aaccaagtaa tccgctggag aaagaacgaa ttcagaatgc aggtggctct |
| 1021 | gtaatgattc agcgtgtgaa tggctctctg gctgtatcga gggcccttgg ggattttgat |
| 1081 | tacaaatgtg tccatggaaa aggtcctact gagcagcttg tctcaccaga gcctgaagtc |
| 1141 | catgatattg aaagatctga agaagatgat cagttcatta tccttgcatg tgatggtatc |
| 1201 | tgggatgtta tgggaaatga agagctctgt gattttgtaa gatccagact tgaagtcact |
| 1261 | gatgaccttg agaaagtttg caatgaagta gtcgacacct gtttgtataa gggaagtcga |
| 1321 | gacaacatga gtgtgatttt gatctgtttt ccaaatgcac ccaaagtatc gccagaagca |
| 1381 | gtgaagaagg aggcagagtt ggacaagtac ctggaatgca gagtagaaga aatcataaag |
| 1441 | aagcaggggg aaggcgtccc cgacttagtc catgtgatgc gcacattagc gagtgagaac |
| 1501 | atccccagcc tcccaccagg gggtgaattg gcaagcaaga ggaatgttat tgaagccgtt |
| 1561 | tacaatagac tgaatcctta caaaaatgac gacactgact ctacatcaac agatgatatg |
| 1621 | tggtaaaact gctcatctag ccatggagtt taccttcacc tccaaaggag agtacagctc |
| 1681 | aactttgttg aaacttttaa catccatcct caactttaag gaaggggata tgacatgggt |
| 1741 | gagaatgatt acatcagaga acttcagcag tacaacagct agcccagaac tgattttttt |
| 1801 | tttttttttt gtaaatttga gacttatgta agcgtgattt caaaccataa ttcgtgttgt |
| 1861 | aaatcagact ccagcaattt ttgttgtatg attttgtttt tttgtaaagt gtaattgtcc |
| 1921 | ttgtacaaaa tgctcatatt taattatgaa ctgctttaaa tcactatcaa agttacaaga |
| 1981 | aatgtttggc ttattgtgtg atgcaacaga tatatagccc tttcaagtca tgttgtgttt |
| 2041 | ggacttgggg ttggaacagg gagagcagca gccatgtcag ctacacgctc aaatgtgcag |
| 2101 | atgattatgg aaaataacct caaaatctta caaagctgaa catccaagga gttattgaaa |
| 2161 | actatcttaa atgttcttgg taggggagtt ggcattgttg ataaagccag tcccttcatt |
| 2221 | taactgtctt tcaggatgtt ccttcgttgt ttccatgagt attgcaggta ataatacagt |
| 2281 | gtattcataa gaatctcaat cttggggcta aatgccttgt ttctttgcac ctcttttcaa |
| 2341 | gtccttacat ttaattacta attgataagc agcagcttcc tacatatagt aggaaactgc |
| 2401 | cacatttttg ctatcatgat tggctgggcc tgctgctgtt cctagtaaga tattctgaat |
| 2461 | tccattttat caataaagct tgatttaaca aacaagaaac ttaatcatgt atgtgtaatt |
| 2521 | cctcttttac cctggccttt taaaacactg tgccgttgta atgagacgtt tctcataggg |
| 2581 | aaagatgtta gtctctttta attggacaac actgtcactc aaggcataga tgaaactttc |
| 2641 | cttccattag aaagactaaa agatttaatt cttggttgta ccttaatcta ttttttaaat |
| 2701 | aggtttcttt caggctgctt atttttcatt aagatgtgta tcagcttgga tttgcctact |
| 2761 | gtttaattaa aatatttatt gtcaaagttt gacaatctaa cactctatgg taggggtgtg |
| 2821 | tgtgtgtgtg tttgtgagtg tgtgtttgcc tgtgattttt aattggccca tgtctttaga |
| 2881 | atccaagtgg ttaagatgta tttgtgattt gaaatatagc atgttgataa tatttagctg |
| 2941 | ttggccttta caaataactt tcaaagctta aggaattgta gatataaaaa taacctaatt |
| 3001 | taatttaggc ttaaattcct ctgattaagc atgtgaaagt aagttttaaa atctgtcgca |
| 3061 | ttgaaaagat tactgttccg tgcccttctg tatttttgtc tctttaggtt gaatattgta |
| 3121 | tttatcacca tgtaatcatt cagtaggcag attcccacta gaaaactgtt gaaatgtaag |
| 3181 | actaaaatac aacattgaat acaaaatcaa aattttgtgt ataaaaacca gtatagtcca |
| 3241 | ttttgttata tttgtttttt ccctaacttg gaaatataca tatttgtata tatagcctta |
| 3301 | aaattaatgt aaagttaggg gagtagtggg gaaagtaatg tgaaatgtct cagatttaag |
| 3361 | tagttaaata ccagcaaaat cttttcatta tccctcttat tttgtgaggt gattaaatgt |
| 3421 | aacttaattg tatttaattt atatcttatt ccagcatgaa tgaggaaaaa ctgaagtact |
| 3481 | atttatattt agaaattcat atcagttgaa attacagaac caattccata cttacaataa |
| 3541 | atacttaatg tctaaatctg tggtagagtg cgaagtatga taatgttcta agttatggct |
| 3601 | ttgcaagcat ctaaatgtgc atttaatgaa taccagtgct tctagtatag actaattacc |
| 3661 | agacatactg gtactgaaag ctaaatccct attataacaa accagttcct taatatttta |
| 3721 | agtagactga caactttagt tccagaaatt gcaaaacttt gaactggact gtgtaatctt |
| 3781 | ttgagatgca aaacttaagt cacaagtaga gtatgtgatg gaaagctgta tttcaaacca |
| 3841 | taacagcata tttagagcct tttttttttg agtctttaaa caagagaaaa ttaaaatatt |
| 3901 | cctgtcaaaa ttattagtat tgaaattagg cttggacacg agagagaacc gtatttgagt |
| 3961 | gatgtgagaa gactaaatct tttccacatg agtcagcact gccatactaa taattttttt |
| 4021 | actataaaaa tacaggaagg aagtatacat tataacagca gactgtgtgt gttcctgatt |
| 4081 | cctggaggta atagtggggg gaaaccaacc atacttttta aaggcacttt tgcacctcta |
| 4141 | ttgtgcactt cattcttgta ccacttaaat tcttcacccc catccccttt ttttgtgcta |
| 4201 | attagcatct cagggcaatg cctcaaaaat gtttgatgtg ttctgttctt tggagggaaa |
| 4261 | aagttcttat gtgatgataa tatagtactc aaaatatact tttatcattt aaatgtctta |
| 4321 | tttgctgcta tgaataggaa ataacatttt gtatagcagg ctctgtttta ccctaacatt |
| 4381 | aaaaaatttc actgatcttt ctttcattaa cagggtagaa tctcctaatt tccactttct |
| 4441 | tgggaatata cttttataga caatagaagc agttctcaat attagcatat actttaaaaa |
| 4501 | atcaaagtga taacttaatt cagctttgga agtatctcaa acatattttt actttatagt |
| 4561 | gcattaactt gcttctagag tacttaatgc aactgctcta gccacttaat tttttatact |
| 4621 | aatctcaaca ttaagaaatt tggattaagt aataaattag ttatgtaatt caagtaatct |
| 4681 | gaattacagc agtactttta gtgatcattc ataggactat atattaaccc agctaataac |
| 4741 | tcagtttttt tacaaaatgt ttcgagtatt attggtaaaa cactgttcta ggctaagcac |
| 4801 | attgggactg taaagaaatg agtagatcct tggcttcaag tttacatctg gacaatttat |
| 4861 | aatctagtgt atgttagtat tataactgga tcactcatca aaaaatatat atatatatct |
| 4921 | attgcccacc tgctatctac caggtactta gctgatcaag gcaggcccct gccctaaaga |
| 4981 | ccttgtttat acttccttta ctcacctgaa aactgttctc cagtttattt tcttctctct |
| 5041 | aaagttaaag agtaattcag aagaaaattt tgcttagcat aagaataaaa ttggactgaa |
| 5101 | gaggcttaag cccattcagt atccttgatt gcatttatcc aacggccttt attcttcctg |
| 5161 | ctgacagcag taactcagag gaataggtag tagatttctg aaaattatcc agccatggaa |
| 5221 | atgtaggtgg ggtttgagtt taaggcattt aaaaatgtaa atatctctag ctaaatttat |
| 5281 | cttaagtaga actctgtgtt tttgtaacac actgccagtg ttaatatcaa attttagcca |
| 5341 | aattattact atgtgtttta atattttaaa ataatttcac tgcccatctt tactggacaa |
| 5401 | actcatttgg agttcaactt gtgatttctg aaagaactga tgaaattggg tactgctttt |
| 5461 | tttctccatt tttcgttttg ttttaatttt gaatttcatg gtatatactt ttagttcaaa |
| 5521 | ctcagctgtt tgtacagtat tgtattagga tttggtatta gaaaagatgt gtaaatatct |
| 5581 | tagtatataa ttgtttctca tttgaggttt ttcttctaag ggaccttaaa gagttttata |
| 5641 | tacttttgct cacagaaact gctggtgaga ttaccatttt ttgagtatct agtcttctag |
| 5701 | tttttctttt aggcattagg aagccttctt tagagttcaa aattttagaa gcctaatttg |
| 5761 | ctcttacttc cttcaattat gtgccatgtg ttttggtttg tatatgtttt aaattgtata |
| 5821 | tttccttgga atatgcttga aatatttaag aatacatttt caaaatgtat aatactgtat |
| 5881 | tgttttgttg atcagaataa taagtctcag ttaaatgttt gttattactg atagtcaaaa |
| 5941 | tgctcaatag aaatgatgag aggcattggt tccaattcat tgtcaaatga acgttttcta |
| 6001 | attttgttca cagattcttt ccctttcgat tgttctgtat gttaagatag tggcttctgc |
| 6061 | tctcactgtt ttcctattta tattactagc aggtaggagt gctaattaga aaaacttaga |
| 6121 | tggtattgaa attacagttg acaacttata tttttatgag atggagaaaa aagattaagt |
| 6181 | tgatataaca acaaagtgga ctttttttct tccttatcct gcacgaaata ttgcccttgt |
| 6241 | ttcctctact ttcctcttgg tgttttctct ttttttcaaa cagaaacagg ccaattccat |
| 6301 | tttcttgagc aagaaagctt agtgtgttac ttcatcaagg ccagctaata ctgtgttaaa |
| 6361 | ccgggctgaa aatgagaaaa cttgggagat ggaggaatgg ggaaatggca gtgggatagg |
| 6421 | tagggaagga ttactcttaa ttgttttaaa agccatagga aagtcttcct tgtacgtggc |
| 6481 | tgtaaattta taagaactat tgtgtcacat aaaccaacaa gaatgaacct ttgctgcttc |
| 6541 | agataatttg atttttccag caaggaaatt aataagttac tgattcttca gcatagaaac |
| 6601 | aactgagaag aattaatgca atgtttcttc actagaaaac ccaacccttc atttcttttc |
| 6661 | attgctccaa aacccagttt tcaactaatg gttttctcat taaactaaat gtttagaaaa |
| 6721 | gttgtttaga gtttttcttt ttcttttaca tagtcctcct gatccagtat aagactattt |
| 6781 | agtaacgtgc atttgtatgg tactatctaa agtaagttag attgatgtaa gagatcgggt |
| 6841 | agctgcggaa caaaattagt tatatcctaa ttaggtacag tgaatgacac aaaatcattt |
| 6901 | tagcaatgct tcttaacctt ttggggtcac aggcgttttg agactgatga atcctaggga |
| 6961 | cttatttacc caggaaaatg cgtatataac atacatatct ccctaaagtt tacaatattg |
| 7021 | tagtggttca tgggccccct ggttaagagc ccattctaaa gtacaatagg gcatcatccc |
| 7081 | ttttcctgca aagcccaaaa gtatatttct agggcatgaa aataacttga gtctatttta |
| 7141 | aggaattgtt tcactctaga ggtagatagg ggacctggct agaatctgac attaaaatat |
| 7201 | actttttaaa aaatattata tttggggtgg ggaaagtgat taaaaggtga aaaaaaaaca |
| 7261 | tagtattcag aagttttgga ggttaatgtc tttctctaag atttgccact ttagaaattc |
| 7321 | aacagaaaag aggtaaaaca gaaatggaat gtatctggaa catttttggc ctccatagtg |
| 7381 | cagatatact atattaacaa gtaatacatt tatttacctg tcagatctcc aggttttaag |
| 7441 | attttgagct ttctagtatt aggattcatt aaatgttcaa ttcatttcat attctaagga |
| 7501 | attaggttat ttacttacta attcaggatg ttaaaataac atccaagtcg gacaaccacc |
| 7561 | accaatgcac acagttaatg agatttctaa aatataataa gtacaatgta acaaacgtat |
| 7621 | agaattttgc atttgttgcc aaaattagat gtttaatgac agcttattta attcccattt |
| 7681 | gtgggacttc tggaacatag aaaccattat cttacctggt tatcccttga ctaaatagca |
| 7741 | tatctgcagg aaaatatctt gtttgtagtg atatgcccca atagtgattg atttcactct |
| 7801 | tgaaatgagt tatatcactt aatttgtata aatgttatga gtggagagac atgtacatgt |
| 7861 | taaaagcatg ttgcattata tattcatttt ttaaactcta taaatgttaa gaataatata |
| 7921 | attgcagaaa tatttttctt aaatacaatg tgtaacaaaa ttctccgtag caactcaccc |
| 7981 | actttgcagt ttatgtgatc cacactttta aagaaattcc ataaatgtat attttgtatt |
| 8041 | atgtattatt tcctggtcca aagaaaatat gtgaattcag ttctaacttt aagaatgtac |
| 8101 | tgtttgtttt caagttcatt gaaaaattgc attcagcctg cgaatggttg cagattgtat |
| 8161 | gttagatgaa aagtagaaat aatttctagt ttggaaaact ggtgccacta aataaacagg |
| 8221 | caattacata a |

In some embodiments described herein, a PPM1A gene product is a PPM1A mRNA isoform transcript (for example, PPM1A mRNA transcript variant 2, corresponding to NCBI Reference Sequence NM_177951.3 (SEQ ID NO: 1911)), or a portion thereof. In some embodiments described herein, a PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA isoform transcript (for example, PPM1A mRNA transcript variant 2, corresponding to NCBI Reference Sequence NM_177951.3 (SEQ ID NO: 1911)), or a portion thereof.

In some embodiments described herein, a PPM1A gene product is a PPM1A mRNA isoform transcript (for example, PPM1A mRNA transcript variant 3, corresponding to NCBI Reference Sequence NM_177952.3 (SEQ ID NO: 1912)), or a portion thereof. In some embodiments described herein, a PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA isoform transcript (for example, PPM1A mRNA transcript variant 3, corresponding to NCBI Reference Sequence NM_177952.3 (SEQ ID NO: 1912)), or a portion thereof.

In some embodiments described herein, a PPM1A gene product is a Mus musculus PPM1A mRNA isoform transcript (for example, Mus musculus PPM1A mRNA alpha isoform transcript, corresponding to NCBI Reference Sequence NM_008910.3 (SEQ ID NO: 1913)), or a portion thereof. In some embodiments described herein, a PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99% identity, or 100% identity with a PPM1A mRNA isoform transcript (for example, Mus musculus PPM1A mRNA alpha isoform transcript, corresponding to NCBI Reference Sequence NM_008910.3 (SEQ ID NO: 1913)), or a portion thereof.

In some embodiments of the disclosure, the PPM1A gene product is a PPM1A mRNA transcript variant other than the PPM1A transcripts described above (e.g., PPM1A mRNA transcript variant 1, corresponding to NCBI Reference Sequence NM_021003.5 (SEQ ID NO: 1910), PPM1A mRNA transcript variant 2, corresponding to NCBI Reference Sequence NM_177951.3 (SEQ ID NO: 1911), PPM1A mRNA transcript variant 3, corresponding to NCBI Reference Sequence NM_177952.3 (SEQ ID NO: 1912), or Mus musculus PPM1A mRNA alpha isoform transcript, corresponding to NCBI Reference Sequence NM_008910.3 (SEQ ID NO: 1913)). In some embodiments, the PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99% identity, or 100% identity with a sequence homologous to a sequence of PPM1A mRNA transcript variant 1, corresponding to NCBI Reference Sequence NM_021003.5 (SEQ ID NO: 1910), PPM1A mRNA transcript variant 2, corresponding to NCBI Reference Sequence NM_177951.3 (SEQ ID NO: 1911), PPM1A mRNA transcript variant 3, corresponding to NCBI Reference Sequence NM_177952.3 (SEQ ID NO: 1912), or Mus musculus PPM1A mRNA alpha isoform transcript, corresponding to NCBI Reference Sequence NM_008910.3 (SEQ ID NO: 1913). In some embodiments, the PPM1A gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a sequence homologous to nucleotides 457-1429 of PPM1A mRNA transcript variant 1, corresponding to NCBI Reference Sequence NM_021003.5 (i.e., nucleotides 457-1429 of SEQ ID NO: 1910), or a portion thereof.

### ATXN2

In some embodiments of the disclosure, the ATXN2 gene product is an ATNX2 mRNA or ATNX2 pre-mRNA comprising sequences from an ATXN2 gene sequence (for example the ATXN2 gene sequence of NCBI Reference Sequence NG_011572.3 (SEQ ID NO: 149355), the ATXN2 gene sequence of NCBI Reference Sequence NC_000012.12 (Reference GRCh38.p13 Primary Assembly), or an ATXN2 coding sequence), or a portion thereof. In some embodiments of the disclosure, the ATXN2 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with an ATXN2 mRNA or ATXN2 pre-mRNA sequence (for example the ATXN2 gene sequence of NCBI Reference Sequence NG_011572.3 (SEQ ID NO: 149355), the ATXN2 gene sequence of NCBI Reference Sequence NC_000012.12 (Reference GRCh38.p13 Primary Assembly), or an ATXN2 coding sequence), or a portion thereof.

| NCBI Reference NG_011572.3 | |
|---|---|
| (SEQ ID NO: 149355) | |
| 1 | ccagcacttt gggaggccaa ggcaggcaga tcacttgagg tgaggagttc tggccaacac |
| 61 | gaaaaaatgt cgtctctact aaaaatacaa aaattagctg gacatggtgg tgagtgccta |
| 121 | taatcccagc tacatggaag gctgagcggg gaggatcact tgaacccagg aggtggaggt |
| 181 | tacagtgaac tgagattgtg ccactgcatt ccagcctggg caagacagca agactctgcc |
| 241 | taaaaacaaa caaacaaatc ccccaaaact aacaaaaatt agctgggtgt ggtggtgcac |
| 301 | gcctgtagtc cctgatactc tgaaggctga gatgggagga ttgctcggga ccgcctggga |
| 361 | ggcagacgct gcagtgagcc agactgtgga actgtactcc agcctggttg gcagagtaag |
| 421 | accctgtctc gaaaaaaaaa aaaaaaagta ttgttttaaa attcaggtgg ggaggatttc |
| 481 | acagtgtctg aacaccagga ggcaagaatc atttgggacc gccatagagg ctgcctgaca |
| 541 | caccacttct tttaaggtgc attactctaa tatatatata tatatatact atttttttga |
| 601 | gatggagtgt cactctgtca ccaggctgga gtgcagtggc atgatcttga ctcactgcaa |
| 661 | cctctgcctg ccaggttcaa gcgattctcc tgcctcagcc tcccgagtag ttaggactac |
| 721 | aggcgcacac caccatgccc agctaatttt tgtattttct tttttttgag acagagtctc |
| 781 | gctctgttgc ccaggctgga gtgcagtggc gccatctcac ctcactgcaa gctccgcctc |
| 841 | ccgagttcat gccattctcc tgcctcagcc tcccaagtag ctgggactac aggcgcccgc |
| 901 | caccacaccc acctattttt ttgtattttt agtagagacg gggtttcact gtgttagcca |
| 961 | ggatggcctt gatctcctga cctcgtgatc tgcccgcctt ggcctcccaa agtgctggga |
| 1021 | ttacaggtgt gagccaccat gcctggctaa tttttgtatt tttaatagag acagggtttc |
| 1081 | accatgttgg caaggatggt ctcgatctct tgacctcagg tgatcagccc acctcagtct |
| 1141 | cccaaagtgc tgggattaca ggcatgagcc accgtgccca gcctactctg atattttcat |
| 1201 | ataaagctta agccttctga agtccagaga aaaggtagtt tccataaata aataaataaa |
| 1261 | ataaaaataa ctaaataaac ttaccacatc acccaacaat ttcgtttgta ggaaccaacc |
| 1321 | caagataaat gaaaacatat atccacatga agatatatgt tcacaaatgt tcatagcagc |
| 1381 | attattattt ttctttcttc ttctttgtgt gtgtgtgtgt gtgttttagc aactctatta |
| 1441 | actgaacgag acagggtctt ggtctgttgc ccaggctaga gtgcagtggc gcattcgtag |
| 1501 | ctcacagcaa cctcgatctc ctgggctcaa gcggtccttc tgcttcagcc tccctagtag |
| 1561 | ctgggactat aggtgcgtgt caccatgacc agctaatttt tttttttttt tttttttgga |
| 1621 | gacagagtat tgctgtgtca cccaggcttc agtgcaatgg tgcaatctcg gctcactgta |
| 1681 | acctccgcct cctgggttca agcaattctc ctgcctcagc ctcccaagta gctgagatta |
| 1741 | caggtgcccg ccaccacgcc tggctaattt tttgtatttt tagtagagac gggatttcac |
| 1801 | catgtaggcc aggctggtct cgaactcctg atctcaggtg atccacccac ctcggcctcc |
| 1861 | caaagtgctg ggattatagg catgagccac cacgcccagc caaccagcta attttttatt |
| 1921 | tttaaagttt ttcatagaga cagggtctct ctatgttgcc caggctggct tcaaactccg |
| 1981 | gggtattcaa gtgatcctcc catctcggta tcctaaagtg ttgggattac aggagtgagc |
| 2041 | caccaatctc agccccagca gcattttcat aatactcaaa aagttgacac aatctaaaca |
| 2101 | tccacaattg gaaagtagac acaaaatgtg gtatgtccat acatggaata tgacatagca |
| 2161 | ataataagga atgaaatact gctacatgcc acaaccttag ataaacctca aaaacattat |
| 2221 | gacaaatgaa agaaggcaga cacaaaaggc tgcatgctgt aagattctac ttatgtgaaa |
| 2281 | aatctggaca aatccataga gagaaaatgt agatgagtgg ttgcctgggg ctaaggatgg |
| 2341 | gaaccaaact gcccaccatc gtgaaggatc tttttggggt aatggaaatg ttctgaaatt |
| 2401 | agtggcaatg gttgtgcaaa tttactgtgt aaatttactg agaatcatta aattgtacac |
| 2461 | aaaataggtg aatttcatag tatgtaaatt atatcttgat aaaactatta agtttttatt |
| 2521 | caatggtagt gagttaaaac aaactttctc agctatgtca gcttcaggcc ttcttttttt |
| 2581 | ttaattttga aaaatttttt atggagagga gggtctcact atgttgccca ggctggtctc |
| 2641 | ctactccttg gttcaaagga tctgcctgcc ttggccttcc aaagtgctgg gattataggt |
| 2701 | gtaggccact gtatccagcc taggccttct tattcaactt ccatcgacct gaaacttact |
| 2761 | attttttttt tgagacggag tctcgctctg tcgcccaggc tggagtgcag tggtgtgatc |
| 2821 | tccgctcact gcaagcaccg cctcccaggt tcacaccatt ctcctgcctc agcctcccaa |
| 2881 | gtagctggga ctacaggtgc gcatcaccac acccagctaa tttttgtatt tttagtagag |
| 2941 | atgggctcta ctaaaaattg gccaggctgg tctcgaactc ctgacctcag gtgatccacc |
| 3001 | ggccactgcc tcccaaagtg ctgggattac aggcgtgagc caccacactg gccaaaactt |
| 3061 | gttcttaaga ttgtattctg ggaccttgat tccaatcaga gaaaagtgat tgtatttttt |
| 3121 | tatttttatt ttttttagat aaagtttcgc tcttgttgcc caggctggag tgcagtggtg |
| 3181 | ccctctttgg tcactgtaac ctccgcctcc tgggttcaag cgattctcct gcctcagcat |
| 3241 | cctgcgtagc tgagatcaca gatgcccacc accacgccca gctaattttt tcgtattttt |
| 3301 | agtagcgatg gggtttcacc atgttggcca cgctggtctt gaactcctga cctcaggtga |
| 3361 | tccatccgcc tcggcctccc agagtgctgg gattacaggt gtgagccacc gcgccaggcc |
| 3421 | aagtgtttgt atttctatta aagaaagaat ataacgggac accattgacg acctgctcca |
| 3481 | ttgcaggcct ccttgctgtt cctcagactc ccccctcaga gcctttgccc tcgctgtgcc |
| 3541 | ctccacctgg agcgtttctc cccaggatcc tcatgcccat gctcatttgg gtccctgccc |
| 3601 | catgtcaccc tctccaggag cttcccctca cagcagccct ggcctgtacc acagccgggt |
| 3661 | acaggtattt ttttgtttca actggttttt tagttccagt ttcctttagg ttactttatt |
| 3721 | tatttattta tttatttatt ttttgagacg gagtctcgct ctgtcgccca ggctggagtg |
| 3781 | catgatctcg gctgactgca acctccacct cccggattca agcaattctc ctgtatcagc |
| 3841 | ctcccgagta gctgggatta caggcgccca ccaccacacc cggctaattt ttatattttt |
| 3901 | ggtagagacg gggtttcacc atgttggcta ggctaggtta atttttaaag ggttttgcaa |
| 3961 | tggtcccttg atctactttt taccttagat gggaaataaa actgatttcc tacattggca |
| 4021 | gaatacaatg atcatttttg cctggactat ctaggaggtt aatttcagct ggactactga |
| 4081 | aaactgctgg ttcaatcatt ctccacgttt atctaagtct ttacctttat ctggacagtt |
| 4141 | ctaggacatt gaggggaatt ttggtgtttc ttcccctatt atttcctgaa gtcatttcac |
| 4201 | tttaaaaaac aatagattca ctgctcaaaa aaaaaaaaaa aagttaccta ctttctactt |
| 4261 | gcttccagtt taactgcaac acattttaaa aagagtctac tgtgctggct gggtaagtta |
| 4321 | aattaaaact tctaaagggt ccaaggtcta aagttcgcac attgttttga ggtcggctct |
| 4381 | gtctctaccg agggagatcc cattatccgt agttctacca gtcccaatcc catatatttc |
| 4441 | ctttagaatc tcatgaatga ggaaaaagaa gttcaagtga gggaacatag gttcaaatga |
| 4501 | aggtcagata cctaaaagag ttttctggtg actgtgcgcg gctggggtgg aaaaagtggg |
| 4561 | gaaaaggtac ccagatgtgg gtggccccgg agggttgctc cactccagcc ccggcagggc |
| 4621 | aggacagcgc ggcctgcctg gtagatgccc cgagccactg gagcgcctac tgtgtggcgg |
| 4681 | gcgggggacg gcaggaaaac ggcaggatgc tgtgtcccct gaatctggca gggttctagg |
| 4741 | tgctttacac gtagcaagac acattctccg ccccaaggca ctcgcagtca gtccattttc |
| 4801 | tgggttgcat caggtggggg caaactaggt ccccgcagaa gtgaagatgc tgaaggaata |
| 4861 | cagtaggaga agaaatgctt ctctcctgtc ctccacacca ggcaggcccc agaggctgag |
| 4921 | accgacacgc cctccccgaa gggcagaccc gccttgagga aggcggatcc gggtagggac |
| 4981 | cgccgcctgg ccctcacccg acccccgaga aagcaaccca gcgcgccgcc cgctcctcac |
| 5041 | gtgtccctcc cggccccggg gccacctcac gttctgcttc cgtctgaccc ctccgacttc |
| 5101 | cggtaaagag tccctatccg cacctccgct cccacccggc gcctcggcgc gcccgccctc |
| 5161 | cgatgcgctc agcggccgca gctcctcgga gtcccgcggt ggccaccgag tctcgccgct |
| 5221 | tcgccgcagc caggtggccc gggtggcgct cgctccagcg gccggcgcgg cggagcgggc |
| 5281 | ggggcggcgg tggcgcggcc ccgggaccgt atccctccgc cgcccctccc ccgcccggcc |
| 5341 | ccggcccccc tccctcccgg cagagctcgc ctccctccgc ctcagactgt tttggtagca |
| 5401 | acggcaacgg cggcggcgcg tttcggcccg gctcccggcg gctccttggt ctcggcgggc |
| 5461 | ctccccgccc cttcgtcgtc ctccttctcc ccctcgccag cccgggcgcc cctccggccg |
| 5521 | cgccaacccg cgcctccccg ctcggcgccc gcgcgtcccc gccgcgttcc ggcgtctcct |
| 5581 | tggcgcgccc ggctcccggc tgtccccgcc cggcgtgcga gccggtgtat gggcccctca |
| 5641 | ccatgtcgct gaagccccag cagcagcagc agcagcagca gcagcagcag cagcagcaac |
| 5701 | agcagcagca gcagcagcag cagcagccgc cgcccgcggc tgccaatgtc cgcaagcccg |
| 5761 | gcggcagcgg ccttctagcg tcgcccgccg ccgcgccttc gccgtcctcg tcctcggtct |
| 5821 | cctcgtcctc ggccacggct ccctcctcgg tggtcgcggc gacctccggc ggcgggaggc |
| 5881 | ccggcctggg caggtgggtg tcggcacccc agccccctcc gctccgggcc cggcgtcccc |
| 5941 | tcccccgcgg cccgcgccgc cgtccccgcc ccgtgacccg ccgggctacc cggggtgggc |
| 6001 | tgggggccgg cagcgcgggg gagactcgct cgggcctgag ccccgaggct cggccggtgg |
| 6061 | gcgcagccgg ggtcctctgg gattgtcagg cctgtccagc ctcccgcagc atccccgccc |
| 6121 | cctcccccgg cggtcaagat ggagggagcg ggcggcctcc cctccccacg cgtgttggga |
| 6181 | ggggttctcg ggtagcggcg atggtcagcc ccggctcccc cttccgcacg atcctccgcc |
| 6241 | cgcagcgtgg ggatgctcgg gcagctcctc cactcccggt ttaggtgtga acgttggagg |
| 6301 | ggtctggagg ctgtggtggc gttttccgga acatgtcccc ctccatgggg gacatctctg |
| 6361 | gaggggagaa gttagggccg cgtcccccgt gccggttaaa ggggtaggca ccgggctcct |
| 6421 | ccggaatcat cagggtctgt cggggctctc tccccgcccc ctccgagtcc tgggaaagat |
| 6481 | cggaggacgg ggtggagaca agtgggcctt ggcccccgca cccctctgcg ttcgtgtccg |
| 6541 | aggcggcggc gggggctccc gaactcccct gaaatcgtgg ggctccatgt ggcctccggc |
| 6601 | agcgttccac cctcccccac ctggggaagg gaaggggtgg ggagtgcccg gccccgtccc |
| 6661 | ggccttcctc cttcccccgc cagacctctc cggcgcgcgg gtggtggccg atccgcattg |
| 6721 | ctgttcgagg ccgcagtgga gaaggcgcct gtggaacatc ggtgggtgag ggctggaccc |
| 6781 | aggctggacc ctggagatcc ggggtggcgg tgctggtggc aggggcgggg caccctgcgc |
| 6841 | acttatccca acccccgccc caatttcgga aatgctagga gagagagatt gcagcagggg |
| 6901 | acgtggtcgg gttcctgaag gcagaaaggc gggtgtttac tagcgtcttt ttccctccta |
| 6961 | agccggggtt gtagtagggg ctgggggctc agtgttgtcc cggctaactg ggtttgactc |
| 7021 | gagggtgtgt ttgtgcagga gggcctgttg ggggtggcgg gcggttgtca gttcgtattt |
| 7081 | cacgaactaa gaaaatgctt agtgttcaaa gggagaagga aacgtcaata gactccattc |
| 7141 | cattgtggcc ggtgtcctta acttcgggag tgccgccaga gcttaccaag ggcacgcaag |
| 7201 | tccatttccc ttgtgcctca agtccatccg tgttgtaggc actactgtgc cttctttagg |
| 7261 | cctaggccgc cggcttgacg gcgggtgacc ggcgtcctcc ttaaataggc atcttgggct |
| 7321 | ttggaaggtg gaataagagg atttttcatt cacccgagtt ttctttttga aaacacattt |
| 7381 | tcagcaaccc atttccaaag aatttttatt tacagcagaa attccccatc aagaggaatc |
| 7441 | agctggtttt taaggaattc tgctgccttc aaagggggcg gaaacagtcg gttatttgac |
| 7501 | tttacacgcc ccgccccccc ttccccttct ctgagtctga agcatcccaa acactactta |
| 7561 | gccaaactag ttcagatgaa gtgatcgttt ccccaagtag ggtaacttca gtttcccttt |
| 7621 | ttcgttggca tctagcgaaa aatgaaaaaa tttaaaatac aacttttata gaaaaggatg |
| 7681 | tattctgttt ttactttctt aggtattagg aagagatttg gcagataatt caacatgttc |
| 7741 | aaatatataa acattaaaac taaggttatt aagttgcatt gactactagg cttaaaaatt |
| 7801 | agattataag agaatttgct cctgagtagt ttgagtgatc aaagatattt ggaatgtttt |
| 7861 | agtaccacaa ggtctttttt ctgttccttg aggctttaca acaatttaag gttaatttag |
| 7921 | atttttcctt gctttaagtt cttttacttg agacctaaat ggcagccctt attctttctg |
| 7981 | atgaataggt gaaattttgt ttactgtgtt ggatttgtgt aatgtgaagt tttattcttg |
| 8041 | aacagatcgt taatgtactt gtagaattac tttgaatttg aatcactttc ctgcattcct |
| 8101 | tgtaaataag tttcagcttc tagaatctcc tcacttaggt ttgtgcgtat caacagtgaa |
| 8161 | aataagtctc tgagagcaag ggtgaaaaaa aatgcagcat tcggtttgac aagtttcgag |
| 8221 | atagcaaaat atgcttgaaa gtctggaaat tcacatctgc tttaagaaac atttcataat |
| 8281 | ttgactttgt gtgtgtgtgt gtgaatagtt tttcatgact ttcagaagtg atttattttg |
| 8341 | ttctttgtta tatatatttt tgaaggtggc tgctttagga aagataatgt aatcacaata |
| 8401 | ttagaacata attttactgt aatctaattt tgtgtgtgtg tgtgtgtgtg tgtgtgtgtg |
| 8461 | tgtgtgtgtg ttttggatgg aatctcactc tgtcgcccag gctggagtgc agtggcctga |
| 8521 | tctcagctta ctgcagtctc tgtctcctgg gttcatttaa gtgattctcc agcctcagcc |
| 8581 | tccccagtag ctgggattac aggttcgtgc taccacacct ggctaatttt tttgtatttt |
| 8641 | tagtgaggac gggattttgc catgttggcc aggctggtct cgaactcctg acctcaagtg |
| 8701 | atccgcctgc cttggcctcc caaagtgctg ggattacagg cgtgagctac tgcccctggc |
| 8761 | caatttttgt atttttagta gagatggggt ttcaccatgt tggccaggct ggtctcgaac |
| 8821 | tcctcctgac ctcaagtgat tcgccagcct cggcctccca aagtgccagg attacaggca |
| 8881 | ggaatgagcc actgccccca accatcagtc taattcttat ttttgctttt taccttttca |
| 8941 | tttttatgta gtagaggtga ttgtgtatgt tattttgtag ttagcttttt tcccctgaac |
| 9001 | gttgtattgt aaatgtaaat tttttttttt ttttttgaga cagagtctcg gtgtttgccc |
| 9061 | agtctgaagt gcagtggtac gatctcagct cactgcagcc tctgactcct gggttcaagc |
| 9121 | gattctccca cctcagcctc ttgagtagct ggggctacag gaatgttcca ccacgcttgg |
| 9181 | ctaatttttg tatttttggt agagacaagg tttcaccatg ttggccagct tggtctcgta |
| 9241 | ctaccgacct caggtgatgc gcccgcctcg gcctcccaaa gtgctgggat tgcaggcgtg |
| 9301 | agccactgcg cccggctgta aggtttttac ttaaccattc tattgttggg aattgggttt |
| 9361 | ccactttttt gttatagata gtggtgcagt gaacattttt aaatagcttt ttgcttcagt |
| 9421 | gtaattattt ccttagagaa agttaccaag agtggtttta ctagttcaga gggcttcagg |
| 9481 | atttttatgg ctcttgctag cggtgctcta ttattcttta gaagacttgt attacttcca |
| 9541 | gtgtcaagaa ggttgctctt ccatggaatg gtttctttgt agtttgtcaa atattgtggg |
| 9601 | gaatttttaa aggaaaaatt gcatttttac tgtcaagtgc atatattact aagtgctttt |
| 9661 | gttagttact ggattattga tatttgagtt taatttggtt cctctgagga tttaataagg |
| 9721 | taatatatgt gaagatgttt tgaaacctgt aaccattatt attaatgagg gtacttggtt |
| 9781 | tatctgtcgt gctgatagta ctgagtaaag tgcaggaatg aaattcctga ggaactgttc |
| 9841 | taaagctttg ttgttgttgt taacctttct ttctcatctg aaagtgtttt ttattagctg |
| 9901 | ctagcctatg accaagttat ttttggtaac ttttttgtaa tttcatggca ctattgggaa |
| 9961 | ttttcgctgg ttgactcttc ttcttctaca ttcccttccc cattaaaaat aaaaatatgg |
| 10021 | atttacaatt gttactctat tcctaaacct aaataatatg acattagaat tgcttgggat |
| 10081 | acaggattca gtctgaataa aatatttttc ttctagtgat tttcagctta gtatttttac |
| 10141 | tgcttctttc tcttgaggca ttgcaactta aaaattgtgc tgtttagcca ggcgcctgta |
| 10201 | atcccagcta cttgggaggc tgaggcagga gcatcacttg agcccaggag gcggaggttg |
| 10261 | cagtcagctg agattgtgcc actgcactcc agcctgggag acagagtgag actctatctc |
| 10321 | aaaaaaaaaa aaaatgtgct gtgatttaat gtagttgttc atcatgcttc catttaaatt |
| 10381 | tcagtgagac tgttcatctt ttgcagttaa atatcttgta gaagggccta aaatatctac |
| 10441 | gttgaataca gctttattga agcatctatg tacatggggt ttttgggatg aatcagtgaa |
| 10501 | taaagcaaac atattgtcct tttggagttt acattctaat gtgactaggc agacaatgag |
| 10561 | acattaaatt accagcctat gtataatagt gtataagagc tatggaatta gaagaaagca |
| 10621 | gattaaaggt atagggagtg tggggagggg aatgagttac aattttaaat ggattggggg |
| 10681 | aacttaattg aggagctaac atttgagcaa agatttgaag gttgggtatt tagccgtttg |
| 10741 | ctttttatct aggttaatta gtcatgtggc ttcattagta atttataagg tttaaatggc |
| 10801 | atcatccttt gttattcttt tatgtgcaca ttgatactaa ccatctctga agttagacca |
| 10861 | aaaaagttaa ttgacattga gggtcattag aggtaaattg tagatggcta ttactaacca |
| 10921 | aagagacatg ttttgttttt cttttgggct tacgtatttt acctaattag tttagttttt |
| 10981 | gtttcaagta tgtggagaaa ataaactttt taagtttggg ccaaaacttg ctttggtttt |
| 11041 | ctttttcttt ttcttttttt ttttttaaga gaaaaatgta agcctgtagt tgcttaaaga |
| 11101 | ttccacattc tgaaacagtg aaaacatggg atcagtcatg gtgttccttt ttttggttaa |
| 11161 | atgtaaactt gtattttcag tgttactcta attagcaatg gtttatactt ctacataagg |
| 11221 | gatgttaact catattgtag ctatttaata gccatatatt ttgacttaaa ggaggatctc |
| 11281 | aaggccaggc gcggtggctc atacctgtaa tcccagcact ttaggaggct gaggcgggtg |
| 11341 | gatcacctgg ggtcaggagt ttgagactag tctggccaac atggtgaaac ccccatctct |
| 11401 | actaaaaata caaaaattag ccgggcatgg tggtgggcgc ctgtaatccc agcttcttgg |
| 11461 | gaggctgagg gaagagaatt gcttgatccc ggaggttgca atgagtgcgg aggttgcagt |
| 11521 | gagctgagat catgccatta cactccagcc tgggcaacag agcgagactc tgtctcaaaa |
| 11581 | caaacaaaca aacaaaaaaa ggaggatctc atttttttgt cctaaatagc tacagccgtg |
| 11641 | ttagaactgt caccttagca aagtattgtt tttttacttt gaaacgaatt ttaaggtttt |
| 11701 | agaagattgt tctctagaat tacaattttc tgttttgact agtgatagta ttttgatgtt |
| 11761 | gtgtaaatag ttgagcatga acaaaaccct attttttttt ttagctattt caagtgattg |
| 11821 | tgacaacttc aacggagatg taaacagttt attaacagtc acacctatta tctttttttt |
| 11881 | tttttttttt ttttgagacg gagtcttgct ctgtcgccca ggctggagtg cagtggcacg |
| 11941 | atctctgctt actgcaacct ttgcctcccg ggttcaagtg attctcctgc ctcagcctcc |
| 12001 | tgagtagctg ggtctacatg cgcacaccac cacgcctggc taatttttgt atttttagta |
| 12061 | gagacagggt ttcaccatgt tggctagaat ggtctcaaac tcctgacctc aggtgatcca |
| 12121 | cctgcctcag cctcccaaag ttctgggatt acaggcatga gccaccgtgc ttggccgctg |
| 12181 | ccgtatcttt ttaaatgaaa gtacttgtgt tttttttgtt tttttccaaa ggatatctgg |
| 12241 | gtcatctatg atgttactgt taccatctaa gggttttttt gtttgttttt gagacagagt |
| 12301 | ctctgtcgcc caggctggag tgcagtggcg tgatcttggc tcactgcaac ctccgcctcc |
| 12361 | caggttcaag caattctcct gccttagccc tcccgaatag ctgggattac aggcacccgc |
| 12421 | caccatgcct ggctaagttt tgcattttta gtagatatgg agtttcacca tgttggccag |
| 12481 | gctgctcttg aactcctgac ctcaggtgat tcgcttgcct cggcctccca aagtgctggg |
| 12541 | attacaggcg tgagccaccc ccgcccagcc tcatgagcta aggtgttttt tttttttttg |
| 12601 | agacagtttt gctctttccc aggctggagt gcagtggtgc aatctcagct cactgcaacc |
| 12661 | tctgtttccc gggttcaagc gattctcctg cctcagcctt ctgagtagct gagattacag |
| 12721 | gtgcctgcta ccccactcag ctaatttttg tatttttagc agagacaggg tttcaccatg |
| 12781 | ttggttaggc tcatctcgaa ctcctgacct taagcgatcc acctgccttg gcctcccaaa |
| 12841 | gtgctgggat tataggcatg agccaccgtg cgcagcctac cctgtctctt aaaaaacagt |
| 12901 | aacaacaaca acaacaacaa aaaatcctaa atcttaaaaa tggaaggcaa aaactctaag |
| 12961 | ctttgagaga ttaggggact tgcccaaagc aatatttgta ggattttatt acacctctcc |
| 13021 | ctttatttat ttttttagag tcaaggtctc cctctgtcac ccaggctgga gtgcagcctc |
| 13081 | aatctatggg gccaagcatt tctcctgtct tagcctcctg agtagctgga actacaggtg |
| 13141 | tacaccagct ggctaacatt taaatttttt gtagagacag ggtcctgcca tgttgcccag |
| 13201 | attggtctca aactcctggg ctcaagtgat cctcctgcct cagcttccca aagtgctgag |
| 13261 | attacaggtg tgagccactg caccgagccc ccccccttta tttttatttt taaattttaa |
| 13321 | gttctggggc ccctcccttg aaataaatag aaacgtaata tatacacaag atcatgctgt |
| 13381 | gtattttaag gcaatggtcc tcaacctttt taacactagg gaccggttct gtggaagatg |
| 13441 | gtttttccat aggggcaggg gatgattttg agatgaaact gttccaccgg ccgggcacgg |
| 13501 | tggctcacgc ctgtaatccc agcactttgg gaggccgagg cgggcagatc acgaggtcag |
| 13561 | gagatcgaga ccatcctggc taacatggtg aaacccccct ctactaaaaa tacaaaaaaa |
| 13621 | ttagctgggc gcggtggagg gcgcctgtag tcacagctac tccggaggcc gaggcaagag |
| 13681 | aatggcatga aacccgggag gcagagcttg cagtgagctg agatagcacc actgtacttc |
| 13741 | agcctggggg acaaagtgag actccgtcta aaaaaaaaaa aattgttcca cctcagatca |
| 13801 | ttatgcattt gttagattct cataaagagc atacaaccta catctcttgc tatatgcagt |
| 13861 | tcccagtagg gtttgtgctt ctataagaac ctaatgctgc acctgatcta acaggtgggg |
| 13921 | ctcaggtgct aatgctcaca cagctcctgt tgtgcagtct ggttcctaac aggcctgttt |
| 13981 | tttttttttt aattagatgg agtctcgctc tgtcaccagg ctggagtgca gtggcacgat |
| 14041 | ctcagctcac tgcaacctct gcctcccggg ttcaagcgat tctcctgcct tagcctccca |
| 14101 | tgtagttggt actacaggcg cacactgtga tgcccagcta atttttgtat ttttagtaga |
| 14161 | gacggggttt caccatgttg gccaggatgg tgtcgatctc ctgaccttgt gatccgccca |
| 14221 | acagcctccc aaagtgctgg aattacaggc gtgagctgct gcgtccggcc ccctaacagg |
| 14281 | cttgttttat ggaatacagt cacggacagt acttgccctt caggatatct ttttgtaacc |
| 14341 | ttgattttgg cttgctaaaa taggaggtct attttctttt ctttgttttt aatgtatgtg |
| 14401 | gttctgtact tacgtggtgt gaaatctaca taaatgttaa atccttggct atttatttat |
| 14461 | tttgagacag agtctcactc tgtcacccag tctggaaagc agtggcataa tctcggctca |
| 14521 | ctgtaacctc cacttcccag gttccagtga ttctcctgcc tcagcctcct gagtagctgg |
| 14581 | gattacaggc atgcaccact acacctggca aatttttgta ttttttttta gtagagatgg |
| 14641 | ggtttcacca tgttggccag gctcgtcttg aactcctgac cttaggtgat ctgcctgtct |
| 14701 | tggcctccca aagttttggg attacagcat gagccactgc gcctcgcctt atttttttga |
| 14761 | gacaggttct agctctgtca cccaggcggg agtgcagtgg tgccatcatg gctcattgca |
| 14821 | acctcgagtt ctcaggccca agtgatcctc ctatctcagc ctcctgagta gctgggacca |
| 14881 | caggcatgcg ccactatgcc cagcaaaatt tttgtttcac tctgttgcct agggtggggt |
| 14941 | gcagtggcag gatatcggct cagtgcaacc tctgcctctt gcgttcaaat gattctcatg |
| 15001 | cctcagcctc ccgagtagct gggattatag gcatgcgcca ctacacctgg ctaatttttg |
| 15061 | tattattggt agagatgggg ttttatcatg ttggccaggc tggtctcgaa ctcccgacct |
| 15121 | caggtgatcc atataccttg gcctcctgaa gtgctggaat tacaggcata agccactgcg |
| 15181 | cctagctttt ttgtttgttt ttattttgta gggacagaga ttttacctgt tgcccaggat |
| 15241 | ggccttgaat tcctgacctc aaacaatttg ccctccttgg cctcccaagg tgctgggatt |
| 15301 | acaggtgtga gccactatgc ctggctggtt ttttaaatta ttattattgt ttgtgtgtgt |
| 15361 | gtgttgcagg atcttaccct gtcacccagg ctggaatgca gtgatgtgat ctcggcttac |
| 15421 | tgcaacctcc gcctcctagg ttcaagtgat tgtcctgcct cagcctcctg agtagctggg |
| 15481 | ataacagctg tgtgccacca tgcctggcta atttttgtat ttttagtaga gatggggttt |
| 15541 | catcatgttg gccaggctgg tctcgaactc ctgaccttag atgatccacc cgcctcgtcc |
| 15601 | tcccaaagtg ctgggattac aggtgtgagc caccgtgacc agtttggttt agtttttttt |
| 15661 | tttttttttt tttttttttt tttttgagaa atctcgctct gtcgcccagg ctagagtgcg |
| 15721 | gtgacacaat ctcagctcac tgcaagctcc acctcccagg ttcatgccat tctcctgcct |
| 15781 | cagcctcccg agtagctggg actacatgcg cccgccacca tgcccggcta atttttttta |
| 15841 | tgcattttaa gtagagatgg ggtttcactg tgttagccag gattgtctca atctcccgac |
| 15901 | ctcttgatct gcccgcctcg gcttcccaaa gtgctgggat tataggcatg agccaccgcg |
| 15961 | tccggcctgg tttggtattt tttttatgag tctgggttgt ttatgaaaac ttgtcacagc |
| 16021 | tgttaacctt aacttttttt ttttcttttt tttccgagac ggagtctcgc tctgtcacct |
| 16081 | aggctggagt gcagtggtgc gatctcggct cattgcaacc tctgcctccc aggttaaagc |
| 16141 | gatttttctg cctcagcctc ctgagtagct gggactgcag gcacgcacca tctcgcctgg |
| 16201 | ctaatttttg tattttagta gagatggggt ttcaccatat tggccaggct ggtctggaac |
| 16261 | ttctggcctc aagtgatcca cctgccttgg ccccccatgc ctggcaacct taacttttta |
| 16321 | tttgctggta attatttgtg tttgcattca tgtgaaaatt tgaaattctc attaacattt |
| 16381 | aaagattctt acatagattg cttgtaattt taaccctgaa gttgtgtcaa gtgactttac |
| 16441 | aatgtcaatt tgttttattt atttatttat ttatttattt atttattttt gtgataggat |
| 16501 | ctggctctgt tgctaaggct ggagtgcagt gttgcaaata cggctcactg caacctctgt |
| 16561 | ctcccgggtt caagccatcc tcccacctca gcctcccaag tagttggaac tactggtgcg |
| 16621 | ccccacagtg cctgcctagt ttttttgtat tttcagtaga tgtggagttt tgccatgttg |
| 16681 | atcttgaact catggcctcg agtgatccac cccacttagg cctcctaaca tgctggtgtt |
| 16741 | acaggtgtga gccactgtgt ccagcccgaa aatgtcagtt tcgtgccatg attaatagct |
| 16801 | aactacattt tgggaatgta ataaaatttc attctataat gaagtctttg taaaactcat |
| 16861 | tagttgtggt atgaggcttg tcggcaatat aagtgaacgt ggtttattct tattaactgt |
| 16921 | atcagaactt tagaatgttg gtctcctgaa accattgcct tgagaggcct tattgaacag |
| 16981 | tgttgccaat gatcagtttt tttttaaatt tccttttttt tgagactgag tcttaccctg |
| 17041 | ttggccaggt tggagtatag tggtatggtc atggctcact gcagcctcaa catcctgggc |
| 17101 | tcaagcagtc ctcctacctc agtctcccga gtagctggaa ctacaggtgt atgccaccat |
| 17161 | gcctggcttt tgtatatttt gtagagacag ggtttagcca tgttgcccag gctggtctca |
| 17221 | aactcttaaa ttcaaatgat ccacccacct agctttccca aagtgcttta attacatgtg |
| 17281 | tgaggcaccg tggctggcca ggtcaaatat ttttcattga cgtttttcat attgcttttt |
| 17341 | aaagtcatgt taaaatattc ttaataattt ttctaagtgg aattaatctt gattataatt |
| 17401 | ttagtttttt ataaagggcg ggttttgaaa caagtactgc atttttcttt tcgggtttat |
| 17461 | aaacatttgc tgtggacttt gtgcagttaa ctattttcat tcctgaaaca catttcgaaa |
| 17521 | tcaggaattg aagactaaat gtcttttcac tgaagcttga gcagatttta gaaaggggag |
| 17581 | ttcttttttt tttttttttt tttggtagaa atgggggtct tgttatgttg cccaggctgg |
| 17641 | cctccaactt ctgggcttaa actgtcctcc tgctttagcc tctggtctgg agagttcttt |
| 17701 | atggcctctt tgagaacttt tactttacac atgattctat ctagctttct tttctgatgt |
| 17761 | acatattggc agcaagtaga aaagcaatgt tttcagaggc agatatatta acagcaatga |
| 17821 | gaaataacag tagcgtgata gaaagttgaa agacttagct gggtgcggtg gctcacgctt |
| 17881 | gtaatcccag cactttggga ggccaaggag ggcggatcac ttgaggtcag gagttcgaga |
| 17941 | ccagtctggc caacatggtg aaaccctgtc tctactgaaa aacagaaaaa gggccgggcg |
| 18001 | tggtggctca cccctgtaat cccagcactt tgggaggttg aggagggcgg attacaaggt |
| 18061 | caagagattg agaccattct ggccaacagg gtgaaacccc atctctacta aaaatacaaa |
| 18121 | aaaattaaat gggcgtggtg atgtgtgcct gtagtcccag ctactcggga ggctgaggca |
| 18181 | ggagaattgc ttgaacccgg gaggcagagg ttgcagtgag ccgagatcgg gccactgcac |
| 18241 | tgacgacaga gggagactcc gtctaaaaaa aaaaaaaaaa aaaaaaaacc agacttgggg |
| 18301 | ctgggcgggc gcctgtaatc ccagctactt gggaggctga ggcaggagaa tcgcttgaac |
| 18361 | ccgggaggtg aaggttgcag tgagctcaga ttgtgccact gtgccccagc ctgggccaca |
| 18421 | gagcagagtg agactctgtc tcaaaaaaaa aaaaaaagtt tggaagactg gtggctgggc |
| 18481 | atggtggctc acacctgtaa tcccaacact ttgggaggct gaagcaggca gattacctga |
| 18541 | gcccaggagt tcaagtccag cctgggcaac acagggaaac cccatctcaa caaaaaatat |
| 18601 | taatacaaaa aatttagcca gtcatggtcg tgcacttctg tagtctcagc tacttgggag |
| 18661 | gctgaggcag gtggttcact taagtctgga tgtcgaggtg agccatgatt gcaccactgc |
| 18721 | actccagcct gggcgttaaa atgagacctt atctcaaaaa aacaaagcaa agagcctggg |
| 18781 | aactactaaa atgggaacta ctaaaaaaca gacacaagag ctcaacaagt ataccattct |
| 18841 | gggaggtttt tttttttttt tttttttttt tttttgagat ggagttttgc tcttgtcacc |
| 18901 | caggctggag tgcaatggcg ccatctctgc tcactgtagt tccgcctccc aggttcaagc |
| 18961 | agttctcctg cctgactcct gagtagctgg gagtacagat attggtcaca caccgggtta |
| 19021 | atttttgtat ttttagtaga gacggggttt ccccattttg gccaggctgg tctcgaactc |
| 19081 | ctgacctcag gtgatccgcc tgcttcagcc tcccaaagtg ccgggaccac aggcgtgagc |
| 19141 | caccgcacct ggcttttttt ttttgacata gaatcttgtt ctgttgccca ggctggagtg |
| 19201 | caatggtaca atcttggccc actgcaacct ctgcctccca gcttctagcg attttcctgc |
| 19261 | ctctgactcc tgagtagctg ggattacggg tgcccgccac cacacccgga taatttttgt |
| 19321 | atttttagta gagatggggt tttgccatat tggccaggcc ggtcttgaac tcctgacctc |
| 19381 | agatgatcca cctgcctagg cctcccaaag tgccgggatt acaggcgtga gccaccactc |
| 19441 | ccggcctggg agttttgact gtaagtttat agctgtatat cttaggccct aagggcatta |
| 19501 | ctgttttata gcacagtgta gttagttaat gtgctcataa tggtgactca taacaccagg |
| 19561 | ttaaatgatt ttttatatct cccaaagaag tatttttcaa tctgcagatc atgacccctt |
| 19621 | agtagattgt gaaacacatt agtggattat gacaagcatt tttagaaaaa tgaaaaagaa |
| 19681 | taagaagtgt taggatgcat tgcattattg aaataattgt ttttgagatg gagtttcgct |
| 19741 | cttagttgcc gaggctggag tgcaatggcc cgatctgcct cccgggttca agtgattctc |
| 19801 | ctacctcagc ctcctgagta gctgggatta cagacatgct ccaccatgcc tggctaattt |
| 19861 | tgtatttagt tttagtagag atggggtttc tccatgttgg tcaggctggt cttgaactcc |
| 19921 | tgacctcagg tgatccactt gcctcggcct cccaaagtgc tggggataca ggcatgaacc |
| 19981 | cctgtgcccg gcctaatttt tgtattttta gtagagatgg ggtttcacca tgttggccag |
| 20041 | gatagtcttg atctcttgac ctcgtaatct gcccacctcg actcccaaag tgctgggatt |
| 20101 | acaggtgtga gccactgcac ccagctgcca agaattgttt taagctttgg tttgagttaa |
| 20161 | tgtatatata ccgcattgta attcaaaatg taatttttgg ccaactctgg gcacattgcc |
| 20221 | tatggactag tcctgctctg ccacgagcag caacagttca atgaattttt tttttttttt |
| 20281 | tttttttttt tttttttttg agacagggtc tctgtcacca aggctagaat gtagtggtgc |
| 20341 | agtctcggct cactgcaacc tctgtttcct gggctcaagc gatcctccca cctcagcctc |
| 20401 | ctgagtagct gggagtacag gagcacgcta ccatgcctgg ctaattttcg tattttttga |
| 20461 | agagatgagg ttttgccatg ttgttcaggc tagtcttgaa ctctggagct cagatgatcc |
| 20521 | acccaccttg gtgtccagaa atgctgggat tacagggatg agccaccgtg cctagccaaa |
| 20581 | aatttttttt taagtaattt tttattgata tagtcaaaaa agttactgct ttagagccag |
| 20641 | agaaacgcag taaaaggatt gagaaagagt tttgaggtta tatctaagct agggttgtca |
| 20701 | gatttggcaa atagaaatac aggacactca gttaaatttg aatttttgat gaacattgac |
| 20761 | cagtttttta gtataattgt gtattaaatt gcatagaaaa aagttattta tctaaagttg |
| 20821 | aaatttaact gagcatcttg tattttatct ggcaactcca gtctaagctg gaatcatggt |
| 20881 | tcactgtttt tttttttttt tttttttttt gagtcggagt cttgctgtgt tgcccaggct |
| 20941 | ggagtgcaat ggtgcgatct tggctcactg caacctccac ctcctgtgct caagtgattc |
| 21001 | tcctgcctca gcctcctgaa tagttgggat tacaggcacc caccaccatg cccagctaat |
| 21061 | ttttatattt ttagtagaga cggggttttc gccatgttgt tcaggctggt cttgaactcc |
| 21121 | tgacctcagg tggtccgccc acctgggcct cccaacgtgc tgggattaca ggcatgatct |
| 21181 | accgtgcctg gccatggttc actcttcagt aactaaaatt taagctctat gaaagcagga |
| 21241 | actttgtttt gttcactatt gattgtatcc ctatttcttg aatggttggc acttaactgc |
| 21301 | ttggtcacat gtttgaatgg gcaagttact cagccactct caggcttagt ttatttacct |
| 21361 | attaaaagag aaagaatatc ttccttggct gggcgcggtg gctcacgcct ataatcccag |
| 21421 | cactttggga ggctgaggcg ggtggatcac gaggtcagga gatcgagacc aacctgggca |
| 21481 | acattgtgaa acctcatctc tactaaaata gaaaaaatta gctgggcatg gtggtgcgca |
| 21541 | tctgtagtcc cagctactcg agaggctgag gcaggggaat cgcttgaacc caggaggtgg |
| 21601 | aggttgcagt gagccaagat tgtgccactg cactccagcc tgggcgacag aacgagactc |
| 21661 | tgtctccaaa aaaaaaaaaa aaacaaacaa aaaaaaaaac tgagatactg gccgggcgcg |
| 21721 | gtggctcgtg cctgtaatcc cagcactttg gaaggccgag gcgggtggat cacgaggtca |
| 21781 | ggagatcgag accgtcctgc ctaacatggg gaaaccctgt ctctactaaa aatacaaaaa |
| 21841 | attagccagg cgtggtggcg ggcgcctgta atcccagcta cttgggaggc tgaggcagga |
| 21901 | gaatggcgtg aacccgggag gcagagcttg cagtgagcgg agatggtgcc actgcactcc |
| 21961 | agcctgctgg gcgacagagc gagactccgt ctcaaacaaa caaacaaaca aacaaaaaaa |
| 22021 | ctgagatact aaagtcttaa tattttctgt ttttatgtat ttattttttg agatgggatc |
| 22081 | ttgctgtatt gcccaggttg gagtacagta ttgtgatcat ggcttattgc agcctttaac |
| 22141 | tcctgggttc aagtgatcct cccacctcag ccccctgagt agctgggacc acaggcacat |
| 22201 | gcaacatcac accctgcagt tctttttttt tttttgagac accgtctcgc tttgtcaccc |
| 22261 | aggctgcagt gcgtggtgca atttctgctc actctaacct ccacctcccg agttcaagca |
| 22321 | gttctgcctc agcctcctga gtagcttggg accacatgtg tgtgccatca tgcctggtta |
| 22381 | attttttgta tttttagtag tgacagggtc ttaccatgtt gcccaggttg gtctcaaact |
| 22441 | cctgagctca agtgatctgc ccgccttcgc ctcccaaagt gtctgcgccc tacaatttaa |
| 22501 | aaaaattttt gtagagacag tctcactgtt acccgggctg gttttgaact tctgccctca |
| 22561 | agtactcctc ttgccttggc ctcccaaagt attgaaatta aggccatgag gcagcacacc |
| 22621 | cagcctaaat tcttcttatg ttctgttctt ggcacatagt agatgttcaa caatgtagag |
| 22681 | tcaaacgcat ttggagttgg aatggctctg gtgttttttt ttttttttta aaccagaaac |
| 22741 | acgtgcagtt tattgaatgc cattgtagaa aagtgtgtga ggataaacgg ctgatagaga |
| 22801 | acttggctct gggggcaggg cgaggaatgg agggtggatg gagtacatgg gaatcagatc |
| 22861 | acgggcagag ctcctggcct agataatgcc tcctgatctg ttgatagact tgaaagatca |
| 22921 | acactgggat gatgctgagc agaatggtcg taatgatgcg cacaatcagg gcccagatgt |
| 22981 | tcaggcactt ggcggtaaag gcataggcct gggccctgat caggtcgcca accatcttct |
| 23041 | tgtccctaga cttcacggag taggccaatg ctatgaagcc caggcagcag gagttcatga |
| 23101 | agtgggtgtt gaacagggac cagacgacat ggtcgggcac ggagttctcg ctgtggatgg |
| 23161 | ggatcacggt ggacattggg ggagcagggt tgtggggtgc ccccagcaca gccacctctt |
| 23221 | gctcctcctt gagcatctca tagttagggg gatggccgat gttggcagga gtgaagaggt |
| 23281 | ttggacattg tggttcatgg tgtccaggga agaccagctg tggtcgggtt gctggggtgg |
| 23341 | ttctcagtgg gcccctccct ttccctggta gtttggattt ctctggctct ggtggttttt |
| 23401 | tagtactcat tctatttacg ggtgaagaaa ttgagaccaa gagggttatt taccagagta |
| 23461 | tctcatcatt ggctgcataa ctggcattag aatctgatgt acttttattt ctaatacatt |
| 23521 | tctttttttt tttttttttt tgagatggag tctcgctctg ttgccgagcc tagagtgcag |
| 23581 | tggggcaatc ttggctcctt gcaacctcca ccccctgggt tcaagctatt cctgtctcag |
| 23641 | cctcccaagt agctgggact acaggcacct gccaccacag ccggctaggt tttgtatttt |
| 23701 | agtagagatg gggtagcacc atgttggcca ggctggtctc gaactcatga cctcaggtga |
| 23761 | tccacctgcc tcggcctccc agtgctggga ttataggcat gagccaccat gcctggcctt |
| 23821 | tctttgtcgt ttcctttctt tctcttcatc cctcctctcc ttttttcccc tccccgctgc |
| 23881 | ctcctcctgt cttcccttct ttccttcctt tctctccttt ttattttttc ctttcttttt |
| 23941 | ctttctctgt ctctcccaac ccttcctctc tccctccctc cctccccttc tctctccccc |
| 24001 | cctccctccc cttctctctc cccctcccct tttgttccta agagacaggg tctccttatg |
| 24061 | ttgctgaggc tgaccttgaa ctcctgagcc cagatgattc tgcctcctta gtagctggga |
| 24121 | ctacacccac ctcccgttcc gttgtcatct tttttttttt tttcttttct ggagacagaa |
| 24181 | tcttcctctg ttgctcaggg tggagtgtag tggcacgatc atagcttact gtaactgtgt |
| 24241 | aacctcgaat tcttgggctc aagcaatcat cccatcatcc cacctcagct tgctgagtac |
| 24301 | ctggggctac aggtgtgtac caccatgtcc ggctaattac ttttcttatt tttaattttt |
| 24361 | cggagatagg atcttgctct gttgcccagg ctggtgtcaa actcctgggc tcaagtgaaa |
| 24421 | ctcttgcctt ggcctcccaa agtgttggga gggattacag gcatgagcca ctgcacccag |
| 24481 | cctcctcttt cttcccattt aactcctaac cacaccgaac tttctgtctg cagagaggag |
| 24541 | cattggtcag cagttcacaa aatggctagg tgtgatggcg tgcacccata gtcccagcta |
| 24601 | cttggggagc tgaggtggga ggatcgctgg agcccaggag ttcaaggccc tgggcaacac |
| 24661 | agcaagacct tatctctggc tgggcccagt ggctcacgcc tgtaatccca gcactttggg |
| 24721 | aggctgaggt gggtggatca cctgaggtca ggagttcgag accagcctgg ccaacatggt |
| 24781 | gagaccctgt gtctactaaa agtacaaaaa ttagccaggc acggtggcgc gctcctgtaa |
| 24841 | tcccagctac tcgggggggc tgagacagga gaatcacttg aacccaggag gaggaggttg |
| 24901 | cagtgaacca agaacacgcc actgcactcc agcctgggtg acatagtgag actcttatct |
| 24961 | caaaaaaaaa aaaaaaaggt cgtctgtact attgcatgtt agtagtttct ttctgcttat |
| 25021 | tgttgagtag tagtctattg tatgcatgta ccagtttgtt catctagtgg tggacattga |
| 25081 | gttagcaggt tttggctatt aaaaataaag ctggaggccg ggtgcgatgt ctcacgcctg |
| 25141 | taatcccagc attttggaag gccgaggcag gcggatcacc taaggttggg agtttgagac |
| 25201 | cagcctgacc aacatggaga aaccccatct ctactaaaaa tacaaaatta gccaggcgtg |
| 25261 | gtggcgcatg cctgtaatcc tagctactca ggaggctgag gcaggagaat cgcttgaacc |
| 25321 | cgggaggcag aggttgtggt gagccaagat tgcaccattg cactccagcc tgggcatcaa |
| 25381 | gagtgaaact ccgtctcaaa aaaataaata aataaagctg gtatgaatat ttatgtacag |
| 25441 | gttttgtgtg aacatatgat tttatttctc ttggttggaa tgcatagaaa tgagattgct |
| 25501 | gggttttgtg gcaagtgttt atttttccag ggtacatata atcctgtgag tgtttattta |
| 25561 | attttaaaag taattgctaa actgtttgct aaagtgactg ctatattttc tttccctagc |
| 25621 | agtgtatgaa tttttttttg aggcagggtc ttgctctgtc acccagggtg gagtgcagtg |
| 25681 | gtgcgatatt gtctgactgc aacattgacc tcctgggctc aagtgatcct cctgcctcag |
| 25741 | cctcctggct gggaccacag gcatgtacca ccacacctgg tagtttgctt tgatttttag |
| 25801 | tagagaagag gtctcactat gttgccctgg gtggtgttga actcctgggc tcaagtgatt |
| 25861 | catctacctc agcctcccaa agtgctggga ttatagatat gagcccctgt gcctggcctc |
| 25921 | attgtggttt taatttgcat ttccctaatg cccagtgata ttgagcattt tttcatgtgt |
| 25981 | ttatttgaca ttcataccat ctttggtgat gagaaactat gtttatgcat tgcttaatga |
| 26041 | tggggatgtg ttttgagaaa ttttttcggt gatcttatca ttgtacaaat atagagttta |
| 26101 | cttacacaag cctagatggt atacctacta gacacatagg ctgtcgtaca gagtattact |
| 26161 | cttaggctac aaatctgtat agcatgttgc ggtactgaac actgttggca gatgtaacat |
| 26221 | aatgttaagt atttgtgaat ctaaacatat ctaaacatag aaaaggtgag taaaaataca |
| 26281 | gcgtaaaaga taaaagtggt atatctgaat aggtcactta ccatgaatgg agcttgcagg |
| 26341 | acaggaagtt gcttgggatg agtcatttat cagtggtgtg tgaatgtgca ggcctaggac |
| 26401 | attactgtat gctactgtag acaaacactg aacagttagg atacactaaa ttgataaata |
| 26461 | tctttcttat tttgtttttt gagatggagt ctcgctctat cgcccaggct ggagtgtagt |
| 26521 | ggcgtgatgt tggctcactg cagtctctgc cttctgggtt caagcgattc tcctgcctca |
| 26581 | acctcctgaa tagctgggat tacaggtgcg tgccaccaca cctggctaat ttttgtattt |
| 26641 | ttagtagaga cgggggtttc accatgttgg ccaggctggt ctcgaactcc tgacctcagg |
| 26701 | tgatccaccc gccgtggcct cccaaagtgc tgggattaca gatgtgagcc accgcacctg |
| 26761 | gccagagatg aggtcttgct gtattgccca gggcgttgaa ctcctgggct ccagcaatcc |
| 26821 | tcccacctca gcttcccacg tagctgggac tgtgggtgca cgccatcatg cctagccgtt |
| 26881 | ttgtgaactg ttgaccaatg ctcttttctg cagacagaaa gttcactgtg gttaggagtt |
| 26941 | aagactttta acctctgacc tcaagtgatc tgcccacctt gacctcccaa agtgctggga |
| 27001 | ttacaggtgt gagccatcac gcctggtcaa aaatatcttt ctttaagagt aaatttacct |
| 27061 | taacttactg gttgatcatt gtatataggt ctgttgttaa ttgaaacatg cgggccgggc |
| 27121 | ccggtggctc atgcctgtaa tcccagcact ttgggaggcc gaggcgggcg gatcacaagg |
| 27181 | tcaggagatc gagaccatcc tggctaacac ggtgaaaccc cgtctctact aaaaatacta |
| 27241 | aaaattaacc gggtgtggtg gcgggcgcct gtaatcccag ctactcggga ggctgaggca |
| 27301 | ggagaatggc gtgaacccgg gaggcggagc ttgcagtgag ccgagatcgt gccactgcac |
| 27361 | tccagcctgg gcaacagagc gagactctgt ctcaaaataa ataaataaat aaataaataa |
| 27421 | ttgaaacatg cggtgcatgt gtttatttgc gatctgactt gtttggaaat atttgcatta |
| 27481 | tcttccttct agatttagag catcttgaca gtaggaacaa gtgttttgta caactttgta |
| 27541 | tgcttagtaa gttatcaatt aacttgtcgt ggccaggcgc agtggctcac gactgtaatc |
| 27601 | ccagcacttt gggaggccga ggcgggcaga tcacctgagg tcaggagttc gagaccagcg |
| 27661 | tggccaacgt ggtgaaaccc tgggtttgtt tgtttgttta tttatttatt tattttttgg |
| 27721 | agacggagtc tcgctctgtc gcccaggctg gagtgcagtg gcgtgatctc ggctcactgc |
| 27781 | aacctccgac tcccaggttc atgccattct cctgcctcag cctcccaagt agctgggact |
| 27841 | acaggagccc gccaccatgc ctggctaatt tttttatttt tagtagagat ggggtttcgc |
| 27901 | cgtgttatct gggatggtct cgaactcctg actttgtgat ccgcccgcct cggcctccca |
| 27961 | aagttctggg attacaggcg tgagccacca cacctggcct accctgtgtt tattacaaat |
| 28021 | acacaaattg gccatttgtg cgtggctcat ctacagtctc agtgactcag aaggctgagg |
| 28081 | caggagaatc tcttgaaccc gggaggcaga ggctgcagtg agcagagatc gtgccactgt |
| 28141 | acttcagcct gggtgacaga gtgagactgt gtctcaaaat aataataata atttgttgaa |
| 28201 | tatgtgactg ttggtttaat ttttattttt atgagatgga gtctcactct gttgcccagg |
| 28261 | ttggagtaca gtggcgtgca gtggcgcaat cttagctcac tgcaacctcc gcctcctgtg |
| 28321 | ttcaggtgat tcagcctccc aagtacctga gactacagac gtgcactacc gtgcctgact |
| 28381 | aatttttgta tttttagtag aaatggggtt tcaccatgtt ggtcagcctg gtctcaaact |
| 28441 | cctattctca agtgatccgc ctacctcgac cttccaaagt ggcggaatta taggtgtgag |
| 28501 | ccgtggtgcc cggccagact attggtttgg ttcggtgtga tgttatgtta tgttatgtta |
| 28561 | tgttatgtta tgttatgtta tgttatgtta ttttaagaca gagtttgtct cttgtcgccc |
| 28621 | aggctggagt gcagcggcat gatctcggct tactgcaacc tccgcctccc aggttcaagt |
| 28681 | gattctcctg tcttagcctc ccaagtagct gggattacag gcgcccacca ccgtgcctgg |
| 28741 | ctaatttttg tatttttagt agagacaggg tttcaccatc ttggccaggc tgttctggaa |
| 28801 | ctcctgacct catgatccac ccgccttggc ctcccaaagt gctgggatta caggcgtgag |
| 28861 | ccactgcgcc tggctgacta ttggttttat tattaagcag tagtagttga ccctgtcatg |
| 28921 | tagaaagcat ggcatttata ggcataccac gtttaatttc ctcccctttt tttatttttg |
| 28981 | gagtacctcc tgcttgtgag gcttgggaat acagtagtga ataagccaga tgaggtctct |
| 29041 | ctctttttgg agcttatgtg gtagtataga ctaggcagaa agttctcatt gcccctgcca |
| 29101 | ccttatggca ttgaggtgtt tgagatgctg atgtttactt ctgtctcata aaatcttgaa |
| 29161 | aggagttctt ttagatgaag aggaaaacaa aatcagaaga atgggcctgg gtcatgtctg |
| 29221 | taaacctccc cacgtcatgg ggaggctgaa atgggaaggg ccaggagttc aagaccaggc |
| 29281 | tgagaaacat aacaagaccc catctctaca aaaaatattt tttaattaat gggggatggc |
| 29341 | agcacacacc tgtagtcgca gctactacga ggctgaagcg agaggattgc ttgagctcag |
| 29401 | gagttaaaga ttgcaggagc tatgatcaca gcactgcgct ccagcccccc ttatcagcag |
| 29461 | tctggtatgt tgctaagggt cttgttcttt ttagtgcttc agggacagcc actggctatg |
| 29521 | cccagaaata agtatgtttg agaagctttc tgacctcagc ttgaaaaatt gattagggtc |
| 29581 | ataattaaaa agggagggaa acaggattga gtgaaccgga cgctaccgtg agtttattct |
| 29641 | cccagggcat acataatctc atgtgattac cacatagccc tgttagataa tctgttatcc |
| 29701 | tgtcctcatt ttacccatga ggaaatgaag gcccagagag gttaaatgac ctattcaaat |
| 29761 | tcactcagaa ggtggcagag atgagttact atcattgtat tttggatctc tggaaagaaa |
| 29821 | gaaaactagt gatggtatta aaaaatgtta ttaatagttt cttttaatca accaggaact |
| 29881 | tgagtcacta gcttctctgg gtgaaggact atacttcaac agtatgaaaa acggaaaaga |
| 29941 | aaatgaggaa ttttggctgg gcacagtggc tcacacctgt aattctagca ctttgggaag |
| 30001 | ccaagggagg agggtcgctt gagctcagga attcaagatc agcctaggca acatagtgag |
| 30061 | gccccatctc tacaaaaata aattagctgg gcatggtggt gcatgcgtat agtctcagct |
| 30121 | acttgggagg ctgactcagg agggtcactt aaacccagga attggaggct gcagtgagct |
| 30181 | atgattgcgc cactgtatac catcccaggc gacagagtga gaccctatcc ccccaccgcc |
| 30241 | aaaaaaaaga aaagaaaatg aggaatttac atttgtgaca gatacggaat tcagggaatt |
| 30301 | tagttgttca tagtctataa atgctataag aagtctccat accttttttt tttttttttt |
| 30361 | ttttttttgg agacagagtc ttgctctgtc gcccaggctg gagtgcagtg gtgcgatctt |
| 30421 | ggctcactac aagctctgcc tctcgggttc acgccattct cctgcctcca cctcccgagt |
| 30481 | agctgggact acaggtgccc gccaccacgc ccggctaatt tttttgtatt tttggtagag |
| 30541 | atgaggtttc actgtgttag ccacagatcc cgacctcatg atctgtctgc ctcagcctcc |
| 30601 | caaagtgctg ggattgcagg cttgaatcac cgcacccggc cggaagtctc catacttttt |
| 30661 | aacccaatct aaaatggtaa ggaaatatat aagaatgtct atttattatt aaattttttc |
| 30721 | tatataaaac atttcagaaa ataaagacta gcatttctga gccaagtggt agtagtggcc |
| 30781 | attttttctg gaaaaaaaaa aaaaaagaaa gaaaaaacac atttagctat ctatgatgtg |
| 30841 | aaaagatgaa cattttattt aggtaataaa tgttatgtca taaaatacca tttattgtgt |
| 30901 | gcctattagg tttcaggaga gctgtgccaa gagcattact tgtatatctt ttaagcctta |
| 30961 | caacagccca gcctgtcagg ctggtagtgc catatctgtt ttacagatga ggaagtgatg |
| 31021 | gattggagaa attaaggaaa ttgcctttag gtcaaagaga taggaagtga caaagctgag |
| 31081 | atttttaacc ttgtgagatt tcaaagtctt tgctttttaa taactgttcc attgcttcta |
| 31141 | atatagagat atgacaaaaa caagtaaaaa tcagtgaaga aggctgggag cagtcgctta |
| 31201 | tgcctgtaat cccagcagtt tgggaggccg aggcgtgtgg atcgcctgag gtcaggaatt |
| 31261 | tgagaccagc ctggccaaca tgacaaaact ccgtctctac taaaaataca aaaaagttag |
| 31321 | ccaggcgtgg tgacaagcac ctgtaatccc agctactcag taggctgagg caaggagaat |
| 31381 | cgcttgaacc tgggaggtgg aggttgcagt gagctgagat cgctccattg cactccagcg |
| 31441 | taggcaacaa agcaagactc cgtctcaaaa aataaataaa taaataaata aaaataataa |
| 31501 | caataatgaa gaaaacaatc cggtgattat tgtcagcaat aaaatttctt caatcaacca |
| 31561 | tgctttagtc ctggcagttc tctatcagtg agtttcaatc aaaaagttcg tttataattt |
| 31621 | tttttttttt aaaattttga aatttggaaa caacatcata aatgatggct agttttctgc |
| 31681 | agctccctat tttggcagat agtctgttgt tactcataat taatttgaac taaaaagtag |
| 31741 | tgttgtacga tatcatgggc tgtgaatgtg tttgtgactt gatctgagaa cccacacacc |
| 31801 | acttaggatg cttctgtagg aaaattagag tatggaactc acttgcccac gctttccctg |
| 31861 | tctcagtcca tgttggtagg ctgcaaagtc tggggctaga aggacactga acaagacttc |
| 31921 | agcagtacat gttagtcttc cagagggaag gaatataata gttgagagaa taattccttt |
| 31981 | cctctgtgac tttaggcaaa ttcttggcta tgctgttatt tatttgggcc aaacaatatc |
| 32041 | aggaggttgt acattttatt cttaattact gcgatacatt aattttatcc atgggtttaa |
| 32101 | cctagcctac cttttgctgt tagacttcaa ctctacttgt gttgggttac ccctctgctt |
| 32161 | aaaaatcacc ctattcccaa gcctgaggga gtctaccttc aaagctttct atgacctaat |
| 32221 | ccaaggcctg tcaaacttcg taaagggcca gatagtaaat ttgttttttt tttttgagat |
| 32281 | ggagttttgc tcttgtcacc caggctggag tgcaatggtg ccatcttggc tcactgcaac |
| 32341 | ctctgcctcc caggttcaag tgattctcct gcctcagcct ctcaagtagc tggggttata |
| 32401 | ggcatgtgcc accacgctcg gctaatttct ttgtatttag tagagatggg ggtttcacca |
| 32461 | ttttggtcag gctggtctcg aactcctgac ctcaggtgat ccacctgccg cggcctccca |
| 32521 | aagtgctggg attaccagtg tgaaccaccg tgcccagccc gatagtaaat attttaggct |
| 32581 | ttgcagtcca tatacagtcc catttttttg tgtatgtttg cacgttttct ttacatattt |
| 32641 | taaaagcccc tttttttttt tttttgagac agagtcttgc tgtgttgctc aggctggagt |
| 32701 | gcagtggtgc aatcttggct cactgcaacc tctgcctcct gggttcaggc gattctcctg |
| 32761 | tctcagcttc ccgagtaact gggattacag gcacatgctg ccacgcccag ctaatttttg |
| 32821 | tatttttagt aaagatgggg tttcgccaca ttggccaggc tggtctcctg atctcaggtg |
| 32881 | atctgcccac ctctgcctcc caaagtgctg ggattacagg tgtgagccac cgtgcctgac |
| 32941 | ctaaaagctc tttacagtgt aaaaaatatt ctgagcttta agccatgtga aaataggcca |
| 33001 | tgggcatttg ctgaccccta atagaactcc attttacctt tctgatcatg tttcccatta |
| 33061 | actcttcaaa aatatgacct ccatttaaat caagatggtc tccttcctca ctgcttgtgg |
| 33121 | aggtccagtg cccagtgtct gcctcttgct tgctcctcca tcattgttct gccattcgag |
| 33181 | atcctcatac ttacccttta agatctagcc caaattttcc atgaaactaa ttctaataat |
| 33241 | taaaaacttc ctgtagaact taactttgtc tagtacaagt tagctttctt attcagtagt |
| 33301 | agcttactat aaattacaag aataaaaaga ttaccatttt ccctcacact gttttgtgga |
| 33361 | gaatgcctaa agttactttt tctttttaca ggtcagtatt cctatttggc atcctaatcc |
| 33421 | cctttcccaa atctgaattt tgggatttga agcttgcatt tgagattatg atttgtcttc |
| 33481 | cttgttgtac acaggagcag ggactttaca attagtattc gcatccctgc tccttcatac |
| 33541 | ttcgtgatgt aaggcaagtt attttcactt atgcttaagt ttcttcccct gtaaaaaggg |
| 33601 | gatggaagag gattaaatga attaaacatg taacacgctt aaagcaatgc ctggcaagta |
| 33661 | ataagtgctc agtaactttt agctgttctt attagcatgt ttggaaacca gtagaaacta |
| 33721 | caccagcaag ttaaggttga aaagtggtat tgatgggctt ggggtagtac agtatgaatg |
| 33781 | gctacagttt agcgtttcat taagtttgta tattcattaa ttcattacac atttgatgct |
| 33841 | gtcagactag gacagagaca aagatgaatg aaacattatc tctgcttcca ggttacccag |
| 33901 | tgtagtagag aaggcaggca tgcagatagt ttaaattggt agcactggga ggggactgcc |
| 33961 | atgggtgggc agtgaagaaa aagggcttca aaataatgag agttgagatg gatcttcaag |
| 34021 | gaagataagc agttttcagt aaggccatga agagaggagg aagttccagg cgggaagagt |
| 34081 | ttgtgctaaa gtacagggat tgctatacac atggtgtatg tagaaaaaat ttggttcaca |
| 34141 | gtgtgataga agaattggag ggggtcctca ctgaaagtaa ggaaacacat ttggaagaat |
| 34201 | atgtttcagt tagaaaatga aatgagctta aagtaaacgc taataaggtt tttaaaatgt |
| 34261 | aaaatttcaa cgtatttaga aagagaacag ctggatgaat cttatgtacc tgtcactcag |
| 34321 | ctttagcagt tatcagtaaa tggccaacgt tgcttcagct atactcccct ctcctccact |
| 34381 | gatagtcttt tgaaggggaa tacaattgtt ttgtggcctc cagaaaggga taagtttatg |
| 34441 | agcaacgggt agatcgttgg gagagacttg agtttcctgt caggaagcat tcttggtgca |
| 34501 | taagtcagag gtgatatgaa tgccgtggaa gggggtggct tactgtctgg agaactcgag |
| 34561 | aagatgggaa tgggcactgt ccagtattgt ggctacttcc acacatggct ctttaaattt |
| 34621 | aaaattatgt tgattaaaat ttaaatattt cagttcctca gccatactaa tcgtatttca |
| 34681 | agtgcttagc tgccacatgt gcctaatggc tgcaatattg gacagcatga cataggacat |
| 34741 | cttcatcatt gtacaaagtt ctcttggaca gcatgggact agagccctaa gatccttttc |
| 34801 | tacctgagtt gtttggattt tttggtgtgt ctaggttgga tctagttgtt catggcttca |
| 34861 | tgaccaagcc ttttatccct ttctctagag ggactcaagg ggtaaaggca ctgaaggggt |
| 34921 | aaaacttcat atgaagagtg tggtggtggt ggtggtgttt taagacaggg tctcgctctg |
| 34981 | tcactcaggc tggagtccag tggcatgatc ctggctcact gcagcctcga cttcctgggc |
| 35041 | ttaagtgatc ctcccacctc agctcccaag taactggaac tgtaggcatg agccaccaca |
| 35101 | cctgcctaat ttttaaaact ttctgtagag acgagatttc gccatgttgt ccaggctggt |
| 35161 | ctcaaattcc tggactcacc ttggcctccc agagtgctgg gattgcaggt gtgggccact |
| 35221 | gcgactggcc ttttttttct cctttactac tctagtgtat gctggaatat gaggaaataa |
| 35281 | ttatattagc tagcagttat taaacactta ataacatacc aggcactgtt ttaagctatg |
| 35341 | cgatctgtat ggaatattac ttaatttcca caaccttatg aaaagatact attttttttc |
| 35401 | ttttgagaag gtactatttt catcttcatt tcatagatgt tgaaattgaa acacagagag |
| 35461 | ctgaagtcac aggattaagg ccacagagct gagaagtgat ggagccggaa tttgaaccca |
| 35521 | agcaattaat gctgatatta gttcttgtgt gaatggtaat tgttttgaaa caatgatcct |
| 35581 | agatgattat atgaccggat taatctggca gtcgttctgt gtgaatttag agttgccttc |
| 35641 | ccacctcagt ttcctaaaaa caaaacaaaa caaaacaaaa caaaaaaaac tctagcttca |
| 35701 | ctgtgtttgg gttgtcatgg cctaccccct cttgccacct catttgactc aactttttag |
| 35761 | ggagaaaata ttcaatacgt ggtataggat ttccctttct aataataatg taaacaacaa |
| 35821 | caagaagtct gaaattggaa gaacaaaatg actcacctaa gtgagttaac cttaagaggt |
| 35881 | ggaacttgat ttctagattt tagttaattg tctaactgat gtactaaata ttagttactt |
| 35941 | aagtattaaa acgggtagac ataatagttg gggagctgct gtagaggggg tagtttgaga |
| 36001 | aggcttcttt caggaggtga catttaagtt ggtaactaac aagaaagggg cagccatgtg |
| 36061 | aatagctgga gggaagagca ttcttacagt tttactggaa gggggttaga ggtatgtggt |
| 36121 | acccttatgc caaagaaaat tagttacttc tatacaacca gtctgattct agaaacctgg |
| 36181 | atcaatgaaa tattttgatt atataaaaaa atctgttacc caggtcttgt tgaaatagca |
| 36241 | ttagaaacta ctgaaggaca tatagaggag gagtgttgaa aaatggtgat ggatgagcag |
| 36301 | aatggtgaaa aataaaaaga catgaagctc tataattata ttgtatggtg acagtaccaa |
| 36361 | tagagattgc atgttttttc tccccagttt ttttttttgt tttgtttttt gtttttgaga |
| 36421 | cagagtctca ctgtgtcact caggctggag tgcattgtcg tgatattggc ttactgcaac |
| 36481 | ctctgcttcc tgggttcaag cgattctcct gcctcagcct cctgagtagc tgggattaca |
| 36541 | ggcatgtgcc accacgcccg gctaattttt gtatttttat ttgagagggg atttcaccat |
| 36601 | gttggcaagg ctggtcttga gttcctgacc tcagataatc cacctgcccc agcctcccaa |
| 36661 | agtgccggga ttacaggtgt gagccactgc gcccggcctc ccccagttgt tgaaacaata |
| 36721 | atggaaggta attttattct tagattattt aatgtttttc agttatcagg atgtgttaga |
| 36781 | ttgtttgtgt atattgtttt gcttgttaat taagtaacac agtgaataag acagacaaac |
| 36841 | atacgaaaat gtacatttat tttatttttt tgagacagtc tgttgcccag gctggagtgc |
| 36901 | agtggcccaa tctcggccca ctgcaacctc tgcctcctga gttgaagcga ttctcttgtg |
| 36961 | tcagcctcat gagtagctgg ggccatgggt gcacgccacc atacccggct aatttttata |
| 37021 | tttttagtag agatggggtt tcaccatatt ggccaggctg gtctcgaatt cctgacctca |
| 37081 | ggtgatctgc ccgccttggt ctcccaaagt gctgggacta caggcatgag ccactgtgcc |
| 37141 | aggccatttc atttttggaa cgttcttttt tttttttgaa atggggtctc gctctgtctc |
| 37201 | ccaggctgga gtgcagtggc tcaatctcag cttactgcaa cctctgcctt ccgggttcaa |
| 37261 | gtgattctcc tgcctcagcc tcctgagtat ctgggactac aggtgcatgc caccacgcca |
| 37321 | ggctaatttt tgtattttta gtagagacgg ggcttcacca tattggtgag gctggtcttg |
| 37381 | aactcctggc ttcgtgatct gcccgcctca acttcgcaaa gtgctgggat tacaagtgtg |
| 37441 | agccaccacg cccggcctgt ttctggaata ttcataatct tttgttgtca tttcaacagt |
| 37501 | gctcacagca gcttcaccag gtgtagattc catcttaaga aaccactttc tttgcttatc |
| 37561 | catgagaagc aacacctcat ctattcaagt tttatcatga gattgcagca attcagttac |
| 37621 | atcttctgac cccacttcta attttagttc tcttgctttt ttaccacatc tgcagttact |
| 37681 | tgctctactg aagtcctgaa cccctcaaaa tcattcatga gtattagaag caatttcctg |
| 37741 | gttgggcacg gtggctcatg cctgtaatcc cagtactttg ggaggccaag gagggcggat |
| 37801 | cacctgaagt caggagttca agaccagtct ggcaaacgtg gtgaaacccc gtttctacta |
| 37861 | aaaatacaaa aattagcggg gatgtggtgg cgggcgccta taatcccagc tacttgggag |
| 37921 | actgaggcag gagaatcgct tgaacctggg aggtggaggt tgcagtgagt tgagattgtg |
| 37981 | cccttgcact ccagcctggg caacaggagc gaaactctat cttaaaaaaa aaaaaaaaga |
| 38041 | aaagcaattt cctctaaaac tcctgttaat gttgatgttt taacctcctc ccatgctcat |
| 38101 | ggatggcatt ctcagtggca tctagaatgg tgaatacttt ttagaaagtt ttcaatttat |
| 38161 | tttgccatca gagaatggct atgaatggca gtagtagcct tacagaatgt atttcttttt |
| 38221 | ttttttttct ttttttttga gatggagttt tttttgctct tgtcacccag gctggagtgc |
| 38281 | agtggcatgc tatctcggct caccgcaacc tccgcctccc gggttcaagc aattctcctg |
| 38341 | cctcagcctc ctgagtagct gggattacag gcatgcacca ccatgcccac ctaattttgt |
| 38401 | atttttagta gaggcggggt ttctccatgt tggtcaggct ggtcttgaac tcccgatctc |
| 38461 | aggtgatctg cctgcctcgg ccttccgaag tgctgagatt acaggcgtga gccaccgcgc |
| 38521 | ccggccgtat ttcttaaata aaatggctta aacgtcaaaa ttatcccttg atccctgggc |
| 38581 | tatggactga ttcttgtgtt agcagttatg aaaacattta tgtccttgta cattcccatc |
| 38641 | atagcttttt gtcaatgaga agtaattttt tttttttttt tgagacagaa tctcactctg |
| 38701 | tttcccagcg tggagtgcag tggcatgatc tcagctcagt gcatcctaca actctgaggt |
| 38761 | tcaagcaatt ctcgtgcctc agcttactga gtagctggga ttacaggcgc ccaccaccac |
| 38821 | gtctggctaa tttttgtatt tttagtagag atggggtttc acgatgttgg ccaggctggg |
| 38881 | ctcgaactcc tggcttcaag tgatccacct gccttggcct cccaaagtgc tgggattgta |
| 38941 | ggtgtgagcc actatgcctg gcctaattgg cctaatttca atattgttat atctcaggga |
| 39001 | atagagaggc acgaggagaa agagagacaa gctgactgct ggttcgtgga gtagtcataa |
| 39061 | cacacaacat ttattaagat tgctgtctta tatggaccgt ttgtggtgcc ttaaaagaaa |
| 39121 | tcagggtaac atcaacgatt actgattaca gattactata acagatacaa taataattgt |
| 39181 | aaattattat ttacaattgt aaaatacaat cttttcttta ttatttacaa ttattgtaaa |
| 39241 | atacaatctg attacagatt actataacgt atacaataat agtggaaaag tttgaaaata |
| 39301 | ttgtgagatt tatgagaatg tgacacaggc gcaaagagag cacatgttac tggaaatacg |
| 39361 | gcactaatgg acttgcccga ctcggggttt ccacagacgg tcagcttgtc aaaaatgcag |
| 39421 | catctgtgaa tttcaataaa gcaaagcaga ataaaatgag gtatgcatgt attgccatca |
| 39481 | catgtacact agtaaaatac gttttttttt tcagtaggtg gatcaacctc aaattttaat |
| 39541 | ataaagcatt acttaaagga gaatatgggg acattcatga catttcttat atgtacataa |
| 39601 | aacttcatga aaataattta atgctatcca gcagtttatt ttagaagtac tggaggctag |
| 39661 | gcatggtgtc ttatgcctgt aatcccagca ctctgggagg ctgaggtagg aggatcactt |
| 39721 | gagttcagga gctggagacc agcttgggca atatagtgcg accccatctc tacaaaagag |
| 39781 | aaaagaagta ctggagtgtt gcagctctta cagaatttgt ctagcaggtt ttccagtctt |
| 39841 | taccagaaat gcccccatgc agaagtagta aatactgatt catgtaaaat aataaacaac |
| 39901 | tttatctttc agtttttaaa agacagggtc ttgtaacgtt gcccagactg gcctttaatt |
| 39961 | cctgggctca agcgatcctc tcacctgagc ctcttgagta gctgagacta caggctgcac |
| 40021 | ctctgcacct ggctctgctt gatttttaat tgctgtattg ctgttgcagc tatgtttttt |
| 40081 | tttttcttca gtgtgaggat gggcaaactt tttatgtaaa gtctcaggta ataagtattt |
| 40141 | taggctctag ggccatatag cttctctgtt gcatatcctt tttttttttt tccatttccc |
| 40201 | ctcaaattcc ttttaccata agcaactctt gaggaacata aaaatcattc ttagcccaga |
| 40261 | agccagacca aaacaggttg tgggctgtag tgtcctgacc cctgatttaa agattgatag |
| 40321 | ctttgaaatg gaaagtttta actttctttt tttttctttc ccttgttctg attgggctgt |
| 40381 | taattcatta ggtatttact cagtgtgtat catatgaggc atgattcctc tgctaatttt |
| 40441 | ggtagtggta gaaagatact tttgccaagc ttggttgtta ggttttcatt tgtccaagag |
| 40501 | ttcctgacca agtgtgaatg gatgttgaaa tcaaggtgtt tctttggcca cacaatgtgc |
| 40561 | ctttgggggc tatatctatg tgcttctggt accttctttt aattttcaca aagacactgc |
| 40621 | ttgccgacca cactgttttg tctaatgtgg ggctatgacc ccctggaaga ggcatcattt |
| 40681 | tctgattttc acagaagcat aatatggtca ggtgatggtc ctgagtagtg ggtatatgac |
| 40741 | agatacacta gtaattataa tacagatcta aactggagag ttgaaaacag catcgtatat |
| 40801 | ttgattgaga taatcgaagg aagacttcct gaaaagatgg catttgagct tcaaggctga |
| 40861 | gtaggattaa gtattattat ttaaaaaatg ccttggacaa tgcattaaat agagttaaca |
| 40921 | aatcacatca cttatagtct ccaattaaaa acattttact taaacataat tttagacttt |
| 40981 | tagaaaaatt gcaaagatat tataaagaat tctcctatat atctcacctg tattcttcaa |
| 41041 | gtaacatttt accatattca ccttaacatt ttctctgtat tggtaattgt atatgtaaga |
| 41101 | tttaatataa aataaaaatt cttattaaac atatgagaga catgatgcct ctttagccct |
| 41161 | aaatacttca acttgtatgt actaataaca agggcattct atttcaaaac cacagtacag |
| 41221 | ttgtcaaaat aaggaaatta ataattgtgt caaactgtta ttctgtttat agaccttcta |
| 41281 | atgtccttta aaacaatcaa caaatcaaca ttcttctggt caagaaccag taaatatgta |
| 41341 | tattctacat atatatatac acatatatat acacacatat attctacata tatatgtgga |
| 41401 | atatacgtat ttactccctc tgtccaagaa ccaatccagg attgttacct tcggttatca |
| 41461 | tgtatctttg gtctccttta atccaaagca gtttctttgt cttttatgac ttgacacttt |
| 41521 | tgaagattac aggttatttt gtagactgtc cctcaactag ggtttatctg aggtttcctt |
| 41581 | atgattagat tcagatattt atttttggca ggaatacaac agaaatgatt tgtgtgtttt |
| 41641 | tctcattgca tgatatcaga aagtgcattg tatatattta tcccattact ggggttgtta |
| 41701 | actttgatca cttggttaga gttgtgtcta ctaagtttct tcactataaa gttatttttc |
| 41761 | acttggtcat ttcatcagta tcttgtgggg agttactttg tggttataca aatactctgt |
| 41821 | ttctactttc ccttactata tttagcttct gtggacactt ttgcctgaaa cagttattta |
| 41881 | ctatggtgtt accaagtagt gatgcccttt tcttccatca ttctgtctac attttttttt |
| 41941 | tttttttttt ttttttgaga tggagtttcg ctcttattgc ccaggctgga gtgcggtggc |
| 42001 | ctgatcttgg ctcactgcaa cctctgcctc ccgggttcaa gcagttctcc tgcgtcagcc |
| 42061 | tcccgagtag ctgggattac agacatgcgc caccactcct ggctaattct gtatgttcag |
| 42121 | tagagacagg atttttccat gttggtcagg ctggtctcca actcccgacc tcaggtgatc |
| 42181 | cacccacctc agcctcccag agtgctagga ttacaggcgt gagctgccac accaggcctt |
| 42241 | ctttttctct tttaagagat agagtcctgc ttcgtcacca aggctggagt gcagtggcat |
| 42301 | gatgatagtt cactgcagcc tcaaactcct gggctcaagt gaacctccca tctgtagctg |
| 42361 | ggactacagg cacctgcata acacctgact gttttttaaa actattttag agatggggtc |
| 42421 | ttgcgaagtt gctcaggatg gtcttgaact ccgggtctta agtggtcctt ctgcctcagc |
| 42481 | ctctggatta gttggcatta caggcatgag ccattgtacc tggcaagtgc atattttctt |
| 42541 | tttttttttt taaggtggag tctcgaggcc gggcgcagtg gctcacacct gtaatcccag |
| 42601 | cactttggaa ggccgaggtg ggtggatcaa gaggtcagga gatcgagacc atcctggcta |
| 42661 | acatggtgaa accctgtctc tactaaaaat acaaaaaatt aactgggcat ggtggcacac |
| 42721 | gcctgtagtc ccagctactc gggaggctga ggcaggagaa ttgcttgaac ccaggaggtg |
| 42781 | gaggttgcag tgagtcaaga tcatgccact gcactccagc ctgagcgaca gaggtagact |
| 42841 | ctgtctcaaa aaaaacagaa agacggagtc ttgctctgtc acccaggctg cattgcagtg |
| 42901 | gcatgaactc cgcctcctga gttcaagcaa ttcttgtgcc tcagcctccc aagtagctgg |
| 42961 | gattacagac atgtgccacc acacgtggct aatttttata gttttagtag aggtggagtt |
| 43021 | tcaccatgtt ggctaggctg gtcttgaact cctgacttca ggtgatccac ccgccttggc |
| 43081 | ctcttgaagt ggtgggatta tgagtgtgag ccactgtgcc cagccaagtg agtatttgct |
| 43141 | tatgtagtat tttaatttta tgattttttt ttctttgaga cggaggttcg ctcttgttgc |
| 43201 | ccaagctgga gtacagtggt gccatctcgg ctcactgcag cctccacccc ctgggttcaa |
| 43261 | gccgttctcc tccctcagcc acctcctcct gaatagttgg gattataggc gcctgccacc |
| 43321 | atgcctggct aattttttgt atatctagta gtgatggagt ttgagcatgt tgccaggctg |
| 43381 | gtcttgaacc tctgacctca ggtgatccac ctgccttggc ctcccaaagt gctgggatta |
| 43441 | aggcatgagc caccatgccc ggccagagac tgctcattta tttttttttt ttgaggcgga |
| 43501 | gtctcgctgt attgcccagg ctggagtgca gtggcacaat ctcggctcac tgcaagctcc |
| 43561 | gcctcccaag ttcacaccat tgtcctgcct tagcctcctg agtagctggg actacaggtg |
| 43621 | cctgccacca cgcctggcta attttgtttt tgtattttta gtagagatgg ggtttcagcc |
| 43681 | cgccttggcc tcctggagtg ctgggattac aggcgtgagt cagggcgcct ggccaatcat |
| 43741 | accttctttt actgcattaa ttatggtttt ctttcgttct taaaacatgt ttatagtgac |
| 43801 | cacttttgaa attcttatta agtcagacat ctggttatac aagcaatttc tattgcctac |
| 43861 | ttctttttcc agtgggtggg gttatacttt cctgtgtctt agcttgtcgt tttttttttt |
| 43921 | gttgttgaaa actggacatt ttaagtaatg tagtaactct ggatacctca ttagcctatg |
| 43981 | gttgggggtg gtggttgtta ctgttatttg cttatttgtc taatgactgg ctgaatgatt |
| 44041 | ttagtgttct atccttcttc cctccctgta cagtgtgaca cgtctgatgc tagttttctt |
| 44101 | gggatgcagc cttgggtatg cctaccatca ctctagaatc acagtgattt tggcatggct |
| 44161 | ttgtctcttt tcctgactgt acccagctgt taagctacac taattactag gtgatgctgt |
| 44221 | gtagtcattt cttggtgtcc ttgggggatt ggtcccagga cccccccgct ggatataaaa |
| 44281 | atttatggat gctctagtcc ctcataaaat ggcacagtat ttgcatatac cggtgcacat |
| 44341 | cctcctgtat gctttgtcat ttctagatta cttataatac ctaatatggt gtaaacacta |
| 44401 | ggtaaatagt tgttatatat tttttatttg tcttattttt attgtattta tttttaagtg |
| 44461 | tttttaatct cgagtgattg aatctgagga tgtgaaatct gcagatatgg agggcctgca |
| 44521 | ttgttttccg tggagctttg ggcctaaact gctccacaga ctgatctgat caaatttgcg |
| 44581 | cttctttgaa gggatagttt ctgagatcag tgcttgaaat ttgttccaat ccacagagga |
| 44641 | gtcctcccag ctctctttcc ctagttctgg ccaccaaact agacaactac aatttagcac |
| 44701 | ttatctccaa tgattctcct cctaccaagt gcctttgaaa gcatcattaa ctctttcata |
| 44761 | ccttgttgca aatgaaattt ctttgggaag agattgtgag ttttttttct cctaaattat |
| 44821 | ggtgcaatat aagtaatata ccattttaac aattttaagt gtattaagtg tttttttttt |
| 44881 | ttgtagtttt tttttttttg ttttttgaga tagtcttgct ctgtcgccca ggctggagtg |
| 44941 | cagtggcacg atctcggctc actggaacct ccacttcccg ggttcaagtg attctctggt |
| 45001 | ctcagcctcc ccaaatatct gggattacag gtgtgcacca ccacgcctgg ctaatttttc |
| 45061 | tatttttagt agaaacgggg tttcaccata ttggtcaggc tggtcttgaa cttctgagct |
| 45121 | cgtgatccac ccacctcggc ctcccaaagt gctgggatta caggccttag ccaccacacc |
| 45181 | tggcctatgc attgctttta tatgtatttt aaaattcata agttctcctc ctatgatgtt |
| 45241 | tttgtcccat gtgatttatt tgttaaaccg tcatctttgg ccgggcgtgg tagctcacgc |
| 45301 | ctgtaatccc agcactttgg gaggctgagg tgggtggatc acaaggttaa gagatcaaga |
| 45361 | ccatcctggc caacatggtg aaaccccgtc tctactaaga atacaaaaat tatctgggca |
| 45421 | tggtgacgcg tacctgtagt cctagctacc tgggaggcgg aggttgcagt gagccaagat |
| 45481 | cgtgccactg cactccagcc tggcgacaga gtgagactct gtctcagaaa aaaaaaaaaa |
| 45541 | caaaaaactg tcatttttta tgttgcattt actgcattct ggatttaaac tgtgaggaac |
| 45601 | ctcatggtat cagttaatat attcttccat cttaatgttt ctcgtaaact ggtagatctg |
| 45661 | taaacttgat taggtctatc ctattgtatc acatcagaag cagaaggtgc tttttttttt |
| 45721 | ttttaaggga aattgtgtga aagtagacag aatggtaaag tgaacccctg cacacctatc |
| 45781 | acccagcttt aatagttatc agctcatacc attcttgttt gatttacaac cccattcatt |
| 45841 | tctcccttct gtattattat tatttagtta attatttttt gagacagggt tttgctctgt |
| 45901 | caccaatgct ggagtgcagt ggcataatca cagctcactg ctgtcttgac ctcctgggct |
| 45961 | caagtgatcc tcccacctca ccctaccaag tagcggggac cacaggcgtg tgccaccatg |
| 46021 | cctggctagt tttttatttt ttgtagaaac agggttttgc tttgttgccc agactgatct |
| 46081 | caaactccgg cactcaagtg atcctcctgc ctcagcctcc taaagtgctg ggattacaag |
| 46141 | catgagctac cacattcagc atgtaaattt ctttatatta atttgactgg cattttaagt |
| 46201 | cacacttgaa tttcatattt ggcaactatt aaaagcatag agtcctggat attagtgttt |
| 46261 | tgttaaacct gatctatcta atcataaata tacttaggtc taaaatatgc tcttggcctt |
| 46321 | tgtttattgc ggttcagtat ttgttactat attaaatagt aaaatattcg gtttgagata |
| 46381 | ctaatgaaaa gattaaaagt aaagcataac ttgaatggat acaaaaagaa acaagaattt |
| 46441 | agacttcagt ggatttcaga gaatactgct tcgatatgct aacattcctg ttgggtgtcc |
| 46501 | aaccgtgtca tagatcagtg gaaattagtg gtttctgcac tttactgtac tgttttttta |
| 46561 | tatgataata ttttcctggt tgaatgattc gttcttttga gtaaactcca tggtcaaaca |
| 46621 | attacttttt attagtcaaa gatgtaacca cataatcact aaaaagaaca gtgtgactta |
| 46681 | tttaaagggg attatgtttt taagtctttt atatagcttt gtagggaggc catatgagtt |
| 46741 | taaggacagt tcgtggcatt tgttcaaggt ttcgtaactt ggcatctcag cagccaccag |
| 46801 | gataccagat catcgttcta agtaagattt aggcatttta gccttcatgt acagactata |
| 46861 | agtacacccc cccacacccc taccaaaact gtaaattcaa atgatgtttg aaaaagcata |
| 46921 | gaatttttgt taggcgaggt agtttattcc ttgtgataca gttccagaga ggcagcataa |
| 46981 | cctaggaatg aaaaacttag acgtggaatc agatacacct ggtttaaata ccagctctac |
| 47041 | tgctcatgaa ctggatgatt ttggtcaaga tacttgactg ctgaggttca gtttcctcac |
| 47101 | ctgtaaagta gaggtgatag attagacatg ttgcatgtga agtacttagt atggtgtctg |
| 47161 | gttttgtagt aagatctata aaagataaat tattagtcat attccttaga cttcaggaat |
| 47221 | ttatctctgt gccatgtttg aggcaaacag ttacagaatt agaatgttag aaatgaaagg |
| 47281 | aatcctagat gtcatttaat tcaagtccat tgttttctgg atgagagaag aaagtgagga |
| 47341 | aaagtgacag agttggagac caagctagga ctggcctcag aatgttaaga gtactcttct |
| 47401 | agggatcgac cagtcgtgtt actagacttt ttggatctga attgtgcttt tccttgaatg |
| 47461 | ttttgaattt tggcttgagt gttgtgatta ttttattaaa atgagattcc agtcctattg |
| 47521 | tcatgactaa tgtttatgag aaatataaca tttcacttta atgatgtttt ttaattattc |
| 47581 | taaggggcct aatctttttc agtggaataa gctttaggtt gtattatatt ctataattca |
| 47641 | cttgaaaata gaattcatct ttacttgaca gccaaatttt gtgtactgca tcttttctga |
| 47701 | gggagagagt tggcaaggaa aggcacttgt tacaacgatc cacacatata gacgcatatt |
| 47761 | atttagaaat gaaagtgctt tgaatgattt agcttatttt cagttttttt ttttctgcag |
| 47821 | ttgtaatcat atgacctgtt tttctttctt tttttttttt tgagacagag tcttgctctg |
| 47881 | tcaccccggc tggagtacaa tggggcggtc tcagctcact gcaacctcca cctcccaggt |
| 47941 | tcaggcgatt cttctgcctc agcctcccta gtagctggga ctacaggcgc atgccaccac |
| 48001 | acctggctaa tttttttatt cttagtagag atggggtttc actgtgttag ccaggatggt |
| 48061 | ctcgaactcc tgaccttgtg atctgcccac ctctgcctcc caaagtgctg ggattacagg |
| 48121 | catgagccac tgcgcccggc ccatatgacc tgtttttctt ttatagatgg gggagaaata |
| 48181 | tgggaagtga cttggtgtca gtcatctgtg ttggttaaat caagaatata atccgtgttt |
| 48241 | tgcttctgaa tagctcttta taacagtgat tggttacttt gggagtaaag attattattt |
| 48301 | agagacagag tcttgctttg tcgcccaggc tagactgcag tggaatgatc gtagcctact |
| 48361 | gcagcctcag actcctggac tctggtgatc ctgcctcagc ctcctgagta gctaggacta |
| 48421 | gaggtgcatg ccacatgcct ggctataatt attattaatt tacgtttagc attagttttt |
| 48481 | ttcttccagt aggctatttt actttattta tttgattttg atgaagtttg attatttcta |
| 48541 | gtttgcttcc ttctatgacc cctacctgtt gtgggtctcc aggcaagcag tgcataggta |
| 48601 | gagccatcct taggtagcct ttagacttaa tattaggtga gctctcccca cagatagcct |
| 48661 | ctcctttatt tgaatggaat tatattttaa gtctggaaat atttttcagc ttatttagcc |
| 48721 | tgttgaattt aataaaaata atatttaatc ttttcagagg tcgaaacagt aacaaaggac |
| 48781 | tgcctcagtc tacggtgagt aactttaatg ttacttattg gggaaaatta gtagctaaaa |
| 48841 | catgatctct aaccacagac caaatgccaa ggcaaaagat tcccttctct tgaattttgt |
| 48901 | catagataac ttgactgttt aagtatgtta ttagcctata tgtgtttttt taatgactct |
| 48961 | gtataaaatg tacaattact tgttgtatta gtccattctt acactgctaa taaagatata |
| 49021 | cctaagactg ggtaatttat aaaggaaaga ggtttaattg actcatgctc tgcattgctg |
| 49081 | gggaggcctc aggaaactta caatcatggt ggaaggggaa gcaaacacat ccttcttcac |
| 49141 | atagcgacag gagagagaag tgctgagcaa agcagggaaa gccccttata aaaccatcag |
| 49201 | atctcctgag aactcactca ctatcatgag agcagcgtag gggaaactgc ccccatgatt |
| 49261 | cagttatctc cacctggtct tgcccttgac acacgagaat tattataatt aaagataaga |
| 49321 | tttgggtggg gacacagaac caaaccatat catttgtaaa tagtatttct gtcacgtgta |
| 49381 | ataacaagaa caagtcgctt gttcttttct aaatgactaa gtgcaaatct aagtgaaaaa |
| 49441 | cctccaaaag atacgtagaa caccaagagt ggagtctgca gagttcttta tgctttttat |
| 49501 | tttgaattaa tgtgcttttt ttctgctgct ttcatttttc tcctttggct ttctggtctt |
| 49561 | aaattttgga atgttatcaa tgaaattgaa ccggacatga agggcagaaa ctataagtcc |
| 49621 | cacatgatgg aagaaataaa tgagaagcta tcacaaattt ttgagacttt gcctttatta |
| 49681 | gattgtttta caagaatcag gaagatatac acgtatatgg tagtaatatg gagtagtgtg |
| 49741 | gttgatcaga cttaagcact gtcactgatg ctgatatgct gggagaacct agtcagggtt |
| 49801 | cttctatgaa ggtatgacct ggcttcctac cccatttatt tatacttcac ccttcttagg |
| 49861 | gtacatttct gtgagtttta acaattgcat acaatcagtg taactaccac cacaatcaag |
| 49921 | ttaatagaac agtttcattg cccaccaaaa tccctcaaat cacttttcag tgaaccctcc |
| 49981 | tctctctcca accattgatt tgtcttccat ccttacggtt tgtgtccttc ctcctctatg |
| 50041 | gaagtttact cttgcttttt tatgtcatgt ttagtcaaaa caccattagt tggtttgact |
| 50101 | gataacactt gaaaacctga ccttctgttc cttctgttct ctatggaagc aaaatattaa |
| 50161 | ataaacaaaa tcttccctta atacatgtaa gatatcataa acctaactaa acattttgca |
| 50221 | acaaataata aacgttagct ttatatgcaa atgtaaatac aggctgagca tccctaatcg |
| 50281 | gaaatgctcc aaaatttcat attttgaatt agggatgttc aagcactaag tataatgcaa |
| 50341 | atatccccaa atccgaaaaa aatccgcagt ctaaaatact tctggtccca agcattttag |
| 50401 | atgaggaaga ttcagtttgt actaatttct aatagttttt ttttttttta atattccaga |
| 50461 | tttcttttga tggaatctat gcaaatatga ggatggttca tatacttaca tcagttgttg |
| 50521 | taagttatta gattattggg gataaactgc cttgggggta gaataaagta attccatgaa |
| 50581 | gttaaaatgt ggataaatga ttgtcaaagt aacattgctt agatcatgct tagtcaggat |
| 50641 | gatttagaga aatagattag aactcctttt atccagtcta atataattca ttgtaaaagt |
| 50701 | acagttggtc ctctgcatct gtgggttcca tattcatgga ttcagccaac cttggatcaa |
| 50761 | aaatatttgt taaaaaggcc aggcacagtg actcacgcct gtaatcccag cactttggga |
| 50821 | gtttgaggtg ggcagatggc ttgagctcac aagtttaaga ccagcctggg caacatggca |
| 50881 | gaactccgtc tctacaaaaa gtaaaaaaac tagccgaacg tggtggtacg tgcctgtagt |
| 50941 | cctagtgact tgggaggctg acgtgggagg attgtttgag cctgggaggt ggaggtttca |
| 51001 | ctgagctgag ataatgcccc tgcactcagc ctggtcaaca gtgccagaca gaccccttct |
| 51061 | caaaaaaaaa aatttttttt tttttttttt tttttttttt ttttgagaaa aaagaggcat |
| 51121 | ggttgcgtct gaaccaaaga tgtacggacg tttttcttgt cattattcct aaaacaatac |
| 51181 | agtatgacaa tttacatagc atttacatta tattaggtat tacaagtaat ttagggatag |
| 51241 | tttaaagtat ttgggagaat gtgcttagtt atatgcaaat actattacat tttatgtaag |
| 51301 | tgacttaagt attatgtaat tcggtatctg aaggaggtcc tggaaccagt cccctaccaa |
| 51361 | taacaacaga tagctgtatt cttgttaacc ctgctgtgtg tgtaaaataa tgttagtagt |
| 51421 | tgattgtctt ttgtacatta ttttgtcact taaaatagct ggggtcagaa atgtttgact |
| 51481 | tcagtattaa aattcgtact gcaaactctg agtagagcct cctgaagaat ttcaagagtt |
| 51541 | cagtgtattg ttaatgtttt gaaatttttt tattgttttg ttagtgaata cctaatattg |
| 51601 | aatgaagcct gatgaggtat aaaaagtaaa atgaaaacaa atatccctgg tgaccgggta |
| 51661 | gtatactgtt tctttgataa ataaattata tgtttttagg gctccaaatg tgaagtacaa |
| 51721 | gtgaaaaatg gaggtatata tgaaggagtt tttaaaactt acagtccgaa ggtaattttt |
| 51781 | actttttttc tttttcttac aaagtaaaag aacattttca tagtcagtgt tttacctagt |
| 51841 | ttttaaagcc actttgaatg attttacttc tcagtttcaa atactgatta ttttatagac |
| 51901 | tggtttgtgt aatcagagag gcttcttgat gtgtgtgctt attaaaatat ttcaaccatt |
| 51961 | tttaagcatt gtgagctaat agagggatgt ggtggtttgt tttttcctct taaaaattat |
| 52021 | tattaatgta cttaagacaa accatagaaa caaaaaacat ttagatatga ggatttttaa |
| 52081 | atgatggaat ggataataga tcatatgcct gggaaaaagg gtatgattct cttgagatta |
| 52141 | tttttgtcaa aggcatataa gaactggtac cttgatgagc taaagaattc ctaacaaatt |
| 52201 | ttattttgta aaggtttgga gtacttactt gtgtttttca ttttagtgtg atttggtact |
| 52261 | tgatgccgca catgagaaaa gtacagaatc cagttcgggg ccgaaacgcg aagaaataat |
| 52321 | ggagagtatt ttgttcaaat gttcagactt tgctgtggta cagtttaaag atatggactc |
| 52381 | cagttatgca aaaagaggtg ggttttgatt tcctaaatat gcctcatggt ttattagatt |
| 52441 | tattcaagca aagattttca cagtgatctt acaaactttt tttaaagaaa tatctgggct |
| 52501 | gggtatggcg gctcattcct gtaatcttag cacttaggga ggctgaggcg ggtggatcac |
| 52561 | ctgaggtcag gagttcgaga ccagcctggc caacatggcg aaaccccgtc tctactaaaa |
| 52621 | atacaaaaat ttatttttgt gtgtggtggc gtgcgcctat agtcctagct actagggagg |
| 52681 | ctgagacaga attgcttgaa cccaggaggc agaggttgca gtgagctgat accgcaccac |
| 52741 | tgcactccag cctgggtgac agagcaagac tccgtttcaa aaaaaaaaag aaagaaaaaa |
| 52801 | gaaatatcta ctttctagaa tagcccaagt aaggtaattt tttagaaaaa tgagaatgtt |
| 52861 | aatgcatttt tgttggaaaa caattagaac tttagagaaa aattaaatag agtttttgtg |
| 52921 | atctcttaaa aaattagttt gtaaagcatt ttctacagtt ttgtggtcaa gaatgctact |
| 52981 | gattatattc aactgaaaat ttcttgtccc atttggccta caatgcttta gtttataagt |
| 53041 | gggcatgtgg caaatctgga aagaaatcaa agtataaggc taaggaagaa aggtagagaa |
| 53101 | cggttggtag aaaacaattg tctaatgaaa atgaaaaagg gtgaagaagt agaacatacg |
| 53161 | tattttaaaa atattcagag tatgagacaa ggctttgaga atttaaaagc gattatgtag |
| 53221 | ttatattaaa aatttagtct ctttttaagt gtccattgat gaacaaagtg ggaattcctg |
| 53281 | ttactcattt gcaaggcatt attgagtgtt cagtaacacg ttgcaaggca cttctgggca |
| 53341 | atcctgaact tggttctcaa attctttttt tttttttttt tgagacggag tcttgttctg |
| 53401 | tcccctgggt ggagtgcagt ggcacgatct cggctcactg cagcctctgc ctcccaggtt |
| 53461 | caagcgattc tcctgcctca gcctcctgag tagctgggac tacaggcgtg tgccaccaca |
| 53521 | ccaagctaat ttttgtattt tttgtagaga cagggtttca ccatgttggc caggatggtc |
| 53581 | tcgattgttt gacctcgtga tccgcccgcc tcggcctccc aaagtgctgg aattacaggc |
| 53641 | atgagccact gcacccagcc ggttctaaaa ttcttttatt tatttgtata tgccaaattc |
| 53701 | tgtagtgaaa tacgtaattc tgttgtaaat tgtagttcag tacaatttga ttttcactat |
| 53761 | tcaaatctat accaaaagct gtttttattg ttgggctgat tcttctacac tgttacttgg |
| 53821 | aaataataat ataccaggat tctttctctt agacttagga gtctttctct ttgcttgctt |
| 53881 | tttcagaggc taacagtact gggtattctt taactgtctt gatatgctga tgaaagcaca |
| 53941 | gtgttctgtt tttgaatctt ctcaaatgtc cttgtctttg attcacaact ttttgtctta |
| 54001 | agaggccttc agcatcccat acaaggaaac aagtcttttt ttagctgcta cctttggagt |
| 54061 | tgattttgtt tatgtctagg agcactaaat tatttatact tatactattg aaatattcct |
| 54121 | ctgttataaa ttcaaaaatt gactttggaa gataaaattt tagttgaatt taatacatag |
| 54181 | cactctggaa agagtattgg ccacaacaaa aaaaaaggtt ccctactcta ttggatacca |
| 54241 | ggtcatttaa cagccattta cggtatgcat tgtctttttg tttttatgat gaattgatat |
| 54301 | ttcccaaatg tggaagagtg aatattactt tgagatgttt gtgatagtcc attccttgct |
| 54361 | cctcttcaaa attaatgtca ttaaattttt attactttat tagatcttca tttctcagat |
| 54421 | aattttagtt cattatagaa aggcaagaaa atacagatca gagtgacaac tttgaaaatc |
| 54481 | tcactctact cataagggga tgggtgtatt ttgctatata ttacaaaatt agttttcttg |
| 54541 | atgaggacat ccactattgg agtaatttca ggtatcttat tttttctttt ctctctcttt |
| 54601 | tttttttttt ttttggagac ggagtttcgc tctgttgccc aggctggagt gcagtggcct |
| 54661 | gatctcggct caccgcaacc tctgcctcct gggttcaagc gattctcttg cctcagcctc |
| 54721 | ccgagtagct ggttactgag gcatgtgcca ccatgcccgg ctaatttttg tatttttagt |
| 54781 | agagacgggg tttcactatg ttggccaggc tggtcttgaa ctcctgacct tgtgatcctc |
| 54841 | ctgccttggc ctcccagagt gctgggatta taggcgtgag ccaccacgcc tgggcaggta |
| 54901 | tcttatttca aaacttacag tggtttagtg aattatacaa ttgcgtccag tgcgtagtat |
| 54961 | cctgaaaata gtattaagtc atgtgtttag gacatcaggt ctcttaagct aagactatcc |
| 55021 | aggcagaaat tgccctcttc tataaaagaa gaaaagtatt aattaggaag tactatcagt |
| 55081 | atggagaaaa ccattttaga attattaatt ggcatggttt ccttcttttt ttttttattt |
| 55141 | cgagatggag tctcactcta tttcccaggc tggagtgcag tggtgcgatc tcggctcact |
| 55201 | gcaacctctg cctcctgggt ttaagcgatt ctcctgcctc agcctcccga gtagctggga |
| 55261 | ttataggcac ataccaccat gccctgctaa tttttttttt tgtttgtatt cttagtacag |
| 55321 | actgggtttc accatgttgg ccaggccgca tggttttcct taataacaaa attaaggcat |
| 55381 | ttattactgc atctagattt tttttatttt ttattagaga cttactcaga ttactcccaa |
| 55441 | agtaaaggaa ggtatggttt aatcaatgct tcttaatgct gggttcacgt ttagtcacct |
| 55501 | ggggagtttt taaaaatgtt ctcacttcta gggatcctgg tttaattata attagcctgg |
| 55561 | gtgaggctct ggacagtcag ggtgtgagct atgggtttca tgtgatgaga tcccaggagt |
| 55621 | ggctctgttc tgtggccttg agaatttgtg ctttctaggc caggtgcggt ggctcactcc |
| 55681 | tgtaatctca ctttgggaga ccaaggtggg cagatcattt gaggtcagga gttcgagacc |
| 55741 | agcctggcca acatgttgaa accccgtctt tactaaaaaa gtaaaaaatt agcgggacgt |
| 55801 | gatggcacat gtctataatc ccagctactt ggggagaggc tgaggcagaa gaatcgcttg |
| 55861 | aacccgggag gcagagattg cgagatcatg ccactgcact ccagcctggg caacagaata |
| 55921 | aaaaaagaat ttgtgcttta ttttcttgcc tcacagtccc ctttctgtct cagaattggc |
| 55981 | aactgcctga aatagtctct gctgttatca tttgatagta cttttccaca tcttgaatgg |
| 56041 | atagatagag tgttttttat aatagaagtg gatgaatgat tagagtatac taatatgaca |
| 56101 | ttgtattttc ctaaaagata tgaattgatt tcatttctga gcttttataa ttctcttctg |
| 56161 | taatagtctg tcaaattatt aaggttgata atattaacta aaatttgagt gcatattcta |
| 56221 | tgtgccagac tctgtgctaa cagatttacc tacatttgtt cacataatca tcacaagttg |
| 56281 | tttctgtagt agatacagct attatccacg tcatagatga ggaaacaggc atatttagga |
| 56341 | aacttgctaa agtgaggaca caaatctagc ttctctactc taactcatgt tcttaacatt |
| 56401 | atactgcagt gacataaatt atgtggtttg gtttgttgtt tatctcagtt gtcataagtc |
| 56461 | gaattaatgt ttgtttgttt gttttgagac agagtcttgc tctgtcgccc aggctgggta |
| 56521 | cagtggcgtg atcttggcgc actgcaacct ccacctcctg ggttcaagca gttatcttgc |
| 56581 | ttcagcctcc ctaataactg ggattacagg cacgtaccac cacacccggg taatttttgt |
| 56641 | atttttagta gagatggggt tttaccatgt tggccaggct gatttcaagc tcctgacctt |
| 56701 | aggtgatcca cccacctggg cctcccaaat tgctgggatt gtaggcatga accactgtgc |
| 56761 | ccagccagta agttccatgg ttgttaaagg atttctccac aaataaagct aaaagtaaaa |
| 56821 | aaaaaaaaaa aaaaaaaaaa ttctcaagca atataagatg cagactatta tgttgttcaa |
| 56881 | gttttttttt ttttttttta atctttggct ttatttttgg ggaaaccttt tttttctttt |
| 56941 | ttgttttcct tgggacggag ttttgctctt gtcgcccagg ctggagtgca atggtgcaat |
| 57001 | cttcgctcac tgcaacctcc gccttctggg ttcaagcgat tctcctatct cagcctcccg |
| 57061 | agtagctggg attacaggca tgtgccacca tgcccggcta actttgtatt tttagtagag |
| 57121 | actgggtttc tccacgttgg tcaggctggt cttgaactcc tgacctcagg tgatccacct |
| 57181 | gcctaggcct cccaaagtgc tgggatcaca agcgtgagcc accgcgccca gccagggaaa |
| 57241 | cctttatttt gaggcggagt ctcgctctgt cacccaggct ggagtgcagt ggcgtgatct |
| 57301 | cagctcactg caacctctgc ttcctaggtt caagcaattc ttctgcctaa gcctcccgag |
| 57361 | gagctgggat tataggcgtc tgccaccatg cccagctaat ttttatattt ttagtagaga |
| 57421 | cggggtttca ccatattggc caggctcttc tcaaattcct gacctcatga tccacccacc |
| 57481 | ttggcctccc aaagtgctag gattacaggc gtgagccacc acactcggct gctggggaaa |
| 57541 | ccttttaaca tgagtaaggt cagtgtgact tttaagttct tgatgctaac atcattgatt |
| 57601 | tcaataaagt ttaaaagtta tattcatgca tatatgcaaa tgaataaaag gctttgaaat |
| 57661 | agtgacttct tacggtacag tgaataagtt tcctttggtc tcttgaatgt tatacatgtt |
| 57721 | ccagtttgat ttactgagaa actgaaagta cctttacgtc atatgagctg tgagtcacct |
| 57781 | tggcacattc ataattagaa gagaccatca gattatcatt ggaaaatcag tttgtattta |
| 57841 | tcctttattt gaattccagt gcagacagat ctgaggttct cttcatttcg ctaaaacttc |
| 57901 | ttagggcctt cagtcgcttt tggctctgta ttcgtgtatc tttggaattg tcctgttatc |
| 57961 | tctgcttgtt ttttacttga ttttccatcc atttccagta ttcctttctc ctctattttt |
| 58021 | ttccttcatt ttctttctgc tcttcctgtt gcgccattat tcatgttttc ctctttactc |
| 58081 | caactcaact atggctttac ttctgtttcc ttattccatt gttcctcata ctttttccta |
| 58141 | ctgcttcatt ttctttgcag tattctcagc ctagatgata ggggtcagca aatctgctca |
| 58201 | tcagtaaata aattttattg tagcatagct atgcccatgc gtttgtgcat tgtctatggc |
| 58261 | tgttttgatg gctgtagcca tagagttgag tagttgtagc tgactgtagg acttgcaaag |
| 58321 | ccagaaaatt tgactgtctc tttacagaaa agcttgccag ctcttggcct aaatcatatt |
| 58381 | ttccgctgca tttagggctt tttaggactg atcaaaaata catgctatac tggctttggt |
| 58441 | gaagtaacag aatgtgctct gtcctttaaa cttacaacta attgcatgct ttgattctaa |
| 58501 | tactgtataa tatcctgcga ttcttattca tgaccattct aattggatct agtctgaaga |
| 58561 | attacttttg cttaacagat tctttgtcac atttagtgaa aaatcataaa aggggaaggt |
| 58621 | tggttaatgg aaaagatctc catcaactaa ccactacctt ccttatctac aaatttatct |
| 58681 | tcttcctccg tgccatcttt tttttttttt ttttcagatg atcttgctct gttgcccagg |
| 58741 | ctggagtgca gtgatgcaat cacagctcac tgcagcctcg acttcccagg ctcaggtgat |
| 58801 | cctctcacct caacctccta cataactggg actgtatgtg cacatcacta tgcctgacta |
| 58861 | attttttata tttatatttt ttgtagagat ggggtttccc tgtattgcac aggctggtct |
| 58921 | caaactgctg ggcctaagag tcttcccacc ttggcctccc aaagtcctgg gattacatga |
| 58981 | gtcaccgcac ccggcctcat tattattttt cctctggttt tagtagagag gatttttaag |
| 59041 | ccaacttcaa tcatgccctt gactctctcc cttctactta cctccttgtt ctctttttct |
| 59101 | ttttcttttt ttttagatgg agtctcggtc tgtcacccag gctgaagtgc agtggcgtga |
| 59161 | tttcagctca ctgcagcctc agcctcctga gtagctgggg ctataggtgc ctgccaccac |
| 59221 | gcccggctaa tttttgtatt tttagtagag atggggtttc accatgttgg ccaggctggt |
| 59281 | ctcgaactcc tgacctcaag tgatcacctg cctcagcctc ccaaagtgct gggattacag |
| 59341 | gcgtgagcca ccacgcctgg ccatcttttt ttttctcctt gctcttttat accacttctc |
| 59401 | tgtttctggg ctcttcaaca tctgcctttc tagttaatct ttccctttag catgaaaacc |
| 59461 | tattcacttc ctgctcatcc taaaaaggat tcttttttgt tttgttttgt ttttgttttt |
| 59521 | gagacagagt ctcgctcttg cccaggctgg agtgcagtgg cactatcttg gctcactgca |
| 59581 | agctccgcct cccgggttca cgccattctc ctgcctcagc ctcccgagta gctgggacta |
| 59641 | caggcacctg ccaccacgcc cagctaaatt ttcgtatttt tagtagagat ggggtttcac |
| 59701 | cgtgttagct aggatggtct cgatctcctg accttgtgat ccatctgcct cggcctccca |
| 59761 | aagtgctggg attacaggca tgagccaccg cactgggccc aaaaggattc tttttaatcc |
| 59821 | tgaattcttc tagccattat cctgcctaag gctacgatta acctctaact gccaggtcct |
| 59881 | ttggaatctt tttctgtctt tattgctgca cttgaatgtt ggtttcaccc tccttcagaa |
| 59941 | tttcctcttc tgtatttttt atgtttattg atcattcctt ccctgcctca ttcctgggct |
| 60001 | tcttttcctt cacacacccc ttagatgtgt gtccccagtg tttgtttctt tgcctgctgc |
| 60061 | tcttgccaca tgacacacac tgccagctac cacacacaag ttccctccta tcatgtgtgt |
| 60121 | atcattgccc ttataccatg ttgtattaaa attatatgct tgtctcccct gttacagttt |
| 60181 | gagctctttg tgctccaagt aaagacagtg atactgtctt tattatttat tctcatggtc |
| 60241 | tagtatagtg ctttggcaca tagtacaggc tcaatataaa tgtgtttgaa taaatgaaat |
| 60301 | tcagtgcctt aatacacttt tgtagaagca ttattttatg gaaagaatga aaaagctgta |
| 60361 | agtggtctta catatatagt catccagcag atacttagag agctctggga tgtgttcctt |
| 60421 | gctgtgcttg ttgctatgga cagtacggag aaatacaaga atctattttg ggtccctttt |
| 60481 | gagaacctag tgaaactgtg tacctagtga aactgtatac cctcacccta gaaaaattta |
| 60541 | cacacatgta gattttacat gtaattcttt taaaaattaa ttttttttct tttttttaaa |
| 60601 | gaaacagggt catgctctgt cactcaggct ggaatgcagt ggtgtgatca tggcttactg |
| 60661 | tagcctcgac ctcctggctc aagcgactct cccacctcag cctcccaagt agctggggct |
| 60721 | acaggtgcac gccgctatgc ccggctaatt tttaaaaata ttttatagac actggttctc |
| 60781 | actatgtttc ccaggctggc ctttacctcc tgggttcaag caatcctcta ccttggcctt |
| 60841 | caaaagtgat gggattatag gtgcaagcca ctgtgcccac gctaatgtaa tttcatggtg |
| 60901 | ttcacagttt cttcagggag ttcatatacg ccatgtactc tattctaagc atttttagag |
| 60961 | ttagagatag caaagcacgt gaataaattc aagaaaaatg gaatgttgta ctgcatgaca |
| 61021 | ttgaatatca aatggagtca gcgatgcaaa taattgtcta gattttacaa aaaaaattag |
| 61081 | cctggtgtgc tggtgtgcgc ctctaatccc agctactcgg gaggctgaga caggagaatc |
| 61141 | atttgaaccc agaaggtgga ggttgcaatg agctgagatc gtaccactgc actccagcct |
| 61201 | gagtgacaga gcgagactcc atctcaaaaa taaaaaataa aagaattgcg tagattttag |
| 61261 | tagttggaag aagttggagt gttaatgtgt aattagagaa cagtgagaaa taaaattcta |
| 61321 | cagattgttt tattctggtg tgctgttgtg ttctcatatg gttgtctttt tggtcttgat |
| 61381 | agtgtatcag taacagagta cgagtaacaa acagggatct cttctgaacg gcgtgacatt |
| 61441 | agaaaagctg tttacggcct caactttgct gtggtttatt aagacacaga tatgtgttca |
| 61501 | ttctggggcc aagcagtaac tggagagtgg cacttattga ggccagtatg gaggcagtac |
| 61561 | agagattatt gagattaaaa gaaagaaaca ggtggaacgg atctatgtaa tggaaagcta |
| 61621 | aacagaatag ttcgtggtac acagtagaaa agcattacat gtttattaag atatggtcat |
| 61681 | cttccattta ttaaagttac atgttttata atttttagag tatatagaaa ttctctaccc |
| 61741 | tatcatgttt gccaaagtca gaacaataac ttcatttatt aaatataaaa aaaataaaaa |
| 61801 | cctctagcat aaaatagaat tttatttgga caaacgataa aaaaatactg tgtggtacta |
| 61861 | gtaagagtaa ggttgattca agatacatgg gagcagaatc caaagtgtag aaataggcca |
| 61921 | ggtgcagtgg ctcatgcctg taatttcaac acctttggag gctgaggcgg gaggatgagt |
| 61981 | tcaggagttc aagactcgcc ttggcaactt ggcaaaaccc catctctaca aaaagtacaa |
| 62041 | aaattagccg ggtgtggtgg tgtactcctg taaacccagc tacttggtgg gctgaggtga |
| 62101 | gaggttcact tgcagccagt aagtcaaggc tgcagtgagc tgtggttatg ccacggcact |
| 62161 | ccagctgggt gacaagcaag accttgtctc aaaaacaaac cagccaggcg tggcggatca |
| 62221 | cctgaggtaa ggagttggag accagcctgg ccgacatggc tctactaaaa atacaaaaat |
| 62281 | tagctgggcg aggtgacggg cacctgtaat cccagctact tgggaggctg aggcaggaga |
| 62341 | atcgcttgaa tccaggagac ggagtttgca atgagccgag atggtggtgc tgcactccag |
| 62401 | cctgggtgac agagccagac tctgtctcaa aaacaaaaat aagcatagga catggggata |
| 62461 | aattgaagat ttatgaagac acagctgaag gagacataaa agtagatttg gctaaatgga |
| 62521 | aacatgccat actttgaatg gaattattta atactacaac gttgtcaatt ttcctcaaat |
| 62581 | aaatctctaa agataatata ttcagttttg gccgggcacg ttggctcacg cctgtaatcc |
| 62641 | cagcactttg gaaggctgag gtgggccgat cacttgagga cgggagtttg agaccagcct |
| 62701 | ggccaacatg gtgaaaccct gtctctacta aaaatacaaa aatcatctgg acatggtggc |
| 62761 | aggtaccagc tacttgggaa gctgaggcag gagaattact cgaaccccgt aggtggaggt |
| 62821 | tgcagtgagc tgagattgca ctccagccgg gtgactccat ctcaaaaaaa aaaaaatttt |
| 62881 | tataatatat atatatatat ccgtttttgt agaaattgac aaaatgattc taaagcttat |
| 62941 | tagattatgt gtattaacag aagaactttg gaaatttttt tccacaagag tcataaagga |
| 63001 | ggacttgccc tacaaaatat gtcagaatta aaacataact tgtcagctgg gtgcggtggc |
| 63061 | tcacgcctat aattccagca ctttgggagg ctgaggcagg cagatcatga ccagcctgac |
| 63121 | caacatggag aaaccccgtc tctactaaaa atacaaaatt agccggtcat ggtggcgcat |
| 63181 | acctgtagtc ccagctactc gggaggctga ggcaggagaa tcgcttgaac tcgggaggtg |
| 63241 | gaggttgcag tgagccgaga tcgcgccatt gcactccagc ctgggcaaca agagtaaaac |
| 63301 | tctgtttcaa aaaaaaaaaa aaaaaaaaaa gaattataac tgtcacagtg gctacgtatg |
| 63361 | gagcatccaa aactgaattt atgtgggtat tttattaata tgcaatatag cactttaatt |
| 63421 | ctggaggaaa ggtggattat tcagtaaatg attctgggac attggggaca aattagatac |
| 63481 | ctacttcaca ctgataaata aaaccaaata gattaatgag aaaactgtga ttaaacaaaa |
| 63541 | caacacccag actacactgg agcaaatctg tgaatttgtt taattttgag tggagaagga |
| 63601 | ctttataagc atgactacca gagcaaaaaa atcatgaagt aaaagatcga tacctttgat |
| 63661 | tataaagaga ttaaagattt aggccgggtg tggtgctcac gcttgtaatc ccagcacttt |
| 63721 | gggaggccaa agcgggtgga tcacttgagg tcaggagttt gagaccaacc tggtcaacct |
| 63781 | ggtgaaaccc catctctact aaaaatacaa aaaaattagt caggcatggt agcacatgcc |
| 63841 | tgtaatccca gctactcagg aggctaaggc aggagaattg cttgaatttg ggaagtggag |
| 63901 | gttgcagtga gccgagattg tgccacatca ctccagcttg ggcgacagag tgactccatc |
| 63961 | tcaaaaaaaa aaaaaaaaaa gacttagacg tgtccaaaag taccatacat ttaaaaagac |
| 64021 | atgccacaaa ctgggaaaag tagaaaaata gttttaaaaa tgaccagtga atgtatgaaa |
| 64081 | aggtggccct cctcacttgt aatgatttaa gaaatgcagt ttatttttat tttattgtat |
| 64141 | ttttaaagaa attcagtttt aaagcagtgg aatatgattg tctatcagct tgcgctgaat |
| 64201 | ggtaaatgtg agaaagatta ctactactta gtggtactga gggagttgca aaacacttaa |
| 64261 | cactgctagt gggatggttt aagtaaaaca agtagcattc ttaaactctc tattaggtaa |
| 64321 | agaataggta agtaatgcat atgtttccag gacattttca gtaagactgt ttactgatag |
| 64381 | ggttgtgtaa tgctaatata cttactatct agttttagta ttattttttt ctcttgtctt |
| 64441 | ggatggtttc aatggagtct tatgcatgca gatatattaa aactagtaat aaagcaagag |
| 64501 | aaggaatgtg gataaattat ctctaatttc tattttgttc tatttctatt tcatactcct |
| 64561 | gggaaagaat attaagtggg catgtgtact tgaacagttg ttctgttttt tattagaaaa |
| 64621 | gaatccgaat ctataaaatg ttttacatat ttgccaggga aacagaaaag atatttgtac |
| 64681 | agctgtaaga attggaatta atttcatttt actgactttt ccttaaccta attctgaaca |
| 64741 | cttttgccat aggtttgaga ataagttgtt ataaaatgac tactattctt cactaatagt |
| 64801 | attggcattt caattcctaa attctgtttt ttgattcttg aacatttctg aatttacttt |
| 64861 | ttttgtctta gttcttctac agaatcattt tcttcttttt tcttttttta tttttatttt |
| 64921 | ttatttttga gacagagtct tgctctgttg cccaggctgg agtgcagtag cgcgatctcg |
| 64981 | gctcactgca agctccgcct cccgggttca tgccattttc tcctgcctca gcctcccggg |
| 65041 | tagctgggac tagaggtacc cgccacagcg cccggctaat tttttgtatt tttagtagag |
| 65101 | acggggtttc accgtgttag ccaaggtggt ctcaatctcc tgacctcgtg atccatccgc |
| 65161 | ctcggcctcc caaagtgctg ggattacagg catgagccat cgcacccggc cttctttttt |
| 65221 | tctttctctt taacttctga gctgaaaata gtacctttta taaagaagtg ctcaaacgat |
| 65281 | gattggactg atttctcctt atttctctct ttctctctgt ctctttcact ctctttttag |
| 65341 | aatttttctt ttttaagtag agacgaggtc ccactatgtt gcccaggctg tcttgaactc |
| 65401 | ctgagcccaa gcaatcctct ttgcctcagc ctcccaaagt gctcggatta caggcttaag |
| 65461 | ctatcacacc aggcctaggc taatttcata ttttgagatg gcacaaattt ctttcaggta |
| 65521 | gctagctttt cctcctcctc cccacttaaa atagatcctg atccagaagc ctaatggaga |
| 65581 | aaatgaaaac agaatgttca cccataaaca gtatctttgt attggaatct tttctaaaac |
| 65641 | ttcttttgat ctttttagga gatagtgtgg gaatcagcaa tctagtatta cgtacgtgga |
| 65701 | atctgtcacc ttgttttttt aaatacagca aacctcatga agtgaatttc catatttttt |
| 65761 | cttgttcttg ttagttttgc accactcagg ctctgctgta gaatttgatg tatatttgat |
| 65821 | tctgtagagc atgggctatt gatcttcact cagctttcag aggaatctga ttagtaagtt |
| 65881 | tgagtttttt attatttttt agttgatttt gaagtaaaat acagcaccat tttaactgat |
| 65941 | accatttcta aacaattttc agttcaaatt ttaagttagc taatttagag cttaagaaaa |
| 66001 | ttgctttaaa aacataaaat tactggctgg gtacagtggc tcattcctgt aatctcagca |
| 66061 | ctttgggagg ccaaggcaga tgaattgctt gagcccagta gttcaagacc agcctgggca |
| 66121 | atatggtgga accccgtttc tacaaaaaaa atacaaaaag tagccagaca cggtggtatg |
| 66181 | tacctgtagt cccagctatt cgggtggcag aggtgagagg atcatctgag cgcagggaga |
| 66241 | ttgaggctgc agtgagccaa gtgagaccct ggtttcaaaa aaaaaaaggt tactaattgc |
| 66301 | agtgcctttt atcttattta atgggcttag tcaaactaag atgatgtatt ttatcttata |
| 66361 | aatgttttcc cttgaatttt aactgaagaa tccaatttgt acctctcaca aacagaatgt |
| 66421 | attagtaagg aaaataaata ctgcttttta ttacttaaat aggatatatt tttctcttag |
| 66481 | ggattttttt tctattttat ctcactttat cgtagtgcta gaaaatttaa tcattcattt |
| 66541 | gagataggga gaaaattagg tttttttttt tcttctattt tgagacaggg tctcattttg |
| 66601 | ttgtccaggc tggagtgcag tggcgccatc gtagctcacc ataacctcaa actcatgggt |
| 66661 | tcaggtgatt caccttagcc tcctgattaa gctgggactg cagatgtgta tcaccactcc |
| 66721 | tggctaattt ttgttgttat tttttgtttg atgaggtctc attatgttgc ccaggctggt |
| 66781 | ctcaaactct gggcctcaaa tgatcctcct gccccagcct cccaaagtgc tgggattaca |
| 66841 | ggcatgaacc tctgctccca gcccattttt taaaatatat tcacagcatt gtgcaaccat |
| 66901 | cactacaatc aatttacatt ttcatcaccc tgaaaagaaa ctctgaaccc cttagcagtt |
| 66961 | cctctctgtt tgtttcaatt ttccccagct ccaggcaact attgatttat tgtcttcata |
| 67021 | ggtttgccca ttctggacat tgcgtattaa tggaatcata taatatatag cctttttttt |
| 67081 | tctttttttt ttttgaaaca gagtctcact gtgtcgccca ggctggagcg cagtggcatg |
| 67141 | attgcagctc actgcatcct ctgcctccca ggctgaagcg attctcctgc ctcagcctct |
| 67201 | tgagtagctg ggactatagg cgcctgccac cacacctact aattttatat ttttagtaaa |
| 67261 | gacggggttg caccatgttg gccaggctgg tctcgaattc ctgacctcaa gtgatctgcc |
| 67321 | cacctcggac tcccaaagtg ctgggattgc agccatgagc caccgcatct ggccatatat |
| 67381 | attatgatag gcttgtttca cttagtatgt ttcttccatg ctgtagcatg tattagtact |
| 67441 | tctttctttt tcatggccaa atattccatt atacagttac acaggtacac tacattttgt |
| 67501 | ttattcatca gttggtggac attttcattg tttccacctt ttgatttata cataatcctg |
| 67561 | ctgcgaacag tgacttttaa agtttttgtg tgggccgggt gtggtggctc atgcctctgt |
| 67621 | aatcccagca ctttgggagg ctggggctgg cagatcattt gaggccggga gttcgagacc |
| 67681 | agcctgccca acatggtgaa accctgtctc tactaaaaat acaaaaatga gctgggtgtg |
| 67741 | gtggcgtgca cctgtaatct cagctactag ggaggctgag gcagagaatc acttgaagct |
| 67801 | gggaagccga ggctacagtg agccgagatc acgccactgc actccagcct gggtgacaga |
| 67861 | gtgaaacttc atctcaaaaa aaaaaaaaaa aaaaaaaaac tgcgtgtgga cataggtttt |
| 67921 | caattctcat gggggtgtgt gtgtatgcat actcatacat acatacacat acctgcaaga |
| 67981 | taattgctgg ctcgtatgct aaatctatgt tgaacctttt acataactgt tgggctgttt |
| 68041 | tgttttcttt ttattatttt ttgaaaatag agttggggtc tcactgttgc acaggctgat |
| 68101 | ttcctgggca tagtggctgt atcattttac aatcctacat agctgtttcc aacgtagctg |
| 68161 | tatcatttta caatcctact agcagtgtct gaggtttctt atgtttttca catcctcacc |
| 68221 | agcatttgtt attgtctgtc tctttgatta tacccatcct agtgggagag taagaagtag |
| 68281 | tatctcactg tagatttttt ttttctgttt acaactttac tttaaaaatt atatatgcac |
| 68341 | acatggtaaa aagttcaaaa cgtgtgtacc aaaagattta acagtgaaaa tagaaaataa |
| 68401 | gtgtggtcct tgttttcttc caccaaggca aatattgtta taatctccta aacaacttgt |
| 68461 | cttccagatt tctcattttc agtcaatctt gggcattgac ataaagaaat tcttagacat |
| 68521 | tgcttttatt agatcatctc atcccttgct caaaatcttc agtggccact gttgtttaca |
| 68581 | gaataaagtt gggatgctat acagggccct tcccagtgga acttctcttt ttcaacctta |
| 68641 | tctctcatta tttcccaatg tttttttttt tttttttgag acggagtctc gctctgtcgc |
| 68701 | ccaggctgga gtgcagtggc gggatctcgg ctcactgcaa gctccgcctc ctgggttcac |
| 68761 | gccattctcc tgcctcagcc tcccaagtag ctgggactac aggcgcccgc cactacgccc |
| 68821 | ggctaatttt ttgtattttt agtagagacg gggtttcacc gttttagccg ggatggtctc |
| 68881 | gatctcctga cctcgtgatc cgcccacctc ggcctcccaa agtgctggga ttacaggcgt |
| 68941 | gagccaccgc gcccggccta tttcccaatg ttaatctact tattgaccta ctaagctggc |
| 69001 | atgttctgtg tgttagacat caccaacttt gtgccttctt tttttgttcg tttttgagtt |
| 69061 | ggagtctcac tctgttgccc aggttggagt gcagtggcgc gatcttggct caccacaacc |
| 69121 | tctgcctccc gggttccagt gattctcctg cctgagcctc ccgagaagct gagacgacag |
| 69181 | gcgcgcgcca ccatgccctg ctaacttttg tatttttagt agagatgggt ttcactgtgt |
| 69241 | ttcccaggct ggtctcgaac tcctgacctt gtgatccacc tgccttgggc tcccaaattg |
| 69301 | ctgggattac aggcgtgagc caccgcggcc ccctgtgcct tcttctttta ctcctggatt |
| 69361 | taatcccaac gtgaagaatc taccttacta actagagttt tagatacttt ttcaaaacca |
| 69421 | agcccacatc tgtccttttt agagtcttct ctgaccttcc ctgctcattg tggtttgttt |
| 69481 | ttattgcctg taacaatggc tgttaaactt tacattttaa attaatttat gtttgtatgt |
| 69541 | atttatttgt tgagaaaggg tctctctctg tcacccctac tagaatgcag tggcgccatc |
| 69601 | atggcttact gcttcctggg ctcaagctgt tctcccattt cagcctcccc atgcaccacc |
| 69661 | ctacctggct aatttttttg tttgtttttt ttagtttagt ttttgtagag acagatgtct |
| 69721 | cactgtgttg cacaggctga tcttgaactc ctgggctcac ttgatcctcc catctcagcc |
| 69781 | tccccaagtg ctgggattac aggtgtgagt caccatgccc agactttaac attttctttt |
| 69841 | tagtatagaa taggtcagtt tttttccctc tgatgagatc ccatgctgac tcttagttaa |
| 69901 | aacaaggctt tggttggaag aagagctagt gatgtcctag ctccctactt actccacttt |
| 69961 | cccttgcctt ctggggtgtc ctgaagacat catagggtgt catgaagtac agttggagaa |
| 70021 | ccagtggtct ccatcatgta ccaaacactc atcttcacga agcagtatgt agtgtctttt |
| 70081 | ttaccggtat attttctctc tcccaatgca ttaaactttt ctggagttca gaaaacaaat |
| 70141 | ttatagaatt aaggaaatgc gtccccccca accatggtgt ctagtatata tacagtgact |
| 70201 | tacagataac aggtgttcaa catatatata ttcctttgat tgatttttga aaagtttaca |
| 70261 | tgtatatatt ttttatatac ggggtctcac tctatcactg aggttggagt gtggtgatgc |
| 70321 | agatcttggc tcaccgcaac ctcctcctcc caggctcaag tgattctccc acctcagcct |
| 70381 | cccgagtacc tgggaccaca ggtgcgcatc accatgcctg gctaattttt tatatttttg |
| 70441 | gtagagacag gattttgccg tgttgcccag gtcggtttcg aactcctgag ctcaggcagt |
| 70501 | ccacctgcct tggcttccca agtgtgagcc accactgaaa tacttatatt tttaaactta |
| 70561 | atttatttat atttattata tttttatgtt tttatatttt aaaaaatatt tttatactca |
| 70621 | ctagacccaa ttttatactc ctaaaccagg gaataactgt ttttttttct cttacatagg |
| 70681 | catgatacca tagacaatga ttaaaattgt aattaccatt catttcttag ttttgtggct |
| 70741 | gggacactga tgtcttcaaa tgttagtttg caaatacagt cagccctctc tatccatggg |
| 70801 | ttacacagct gtgaattcaa ccaaccatgg atccaaaata tatgggaaat acgctggggc |
| 70861 | tgtgggtcac acctgtaatt ccagcactta gggaggctga ggcagatgga tcacctgagg |
| 70921 | tcaggagttc aagaccagcc tggccaacat ggcaaaaccc tagctctact ataagtacaa |
| 70981 | aaaattagct ggccatggta gtgcacatgt gtaatcccag ctactcgaga ggttgagaca |
| 71041 | agcaatttgc ttgaacctga gaagtagagg tttccatgag ctgagattgt gtcactgcac |
| 71101 | tccagcctgc gcaacagagt gtgagaagaa aagaaaaaaa actgtctgaa aagaaaaaaa |
| 71161 | aaaattatat gggaaatcaa aagcatctat actgaacatg tacagacttt ttttcttgtc |
| 71221 | attattcctt aagcagtacc acaactattt ccgtagcatt tactttgtat taggtattat |
| 71281 | aggtaaccta gaggtttaaa gtatgcgaga gtatgcaaat actacaccac tttgtatcag |
| 71341 | ggacttaagc atccctggat tttggtatcc ctagggggta ttagaaccaa tcccccatag |
| 71401 | atgctgaagg acaactgtag tgtgtgttgg aataatttat tttcaaatgg atcatttgga |
| 71461 | gaacactatt ctttaggaaa catagcctcc taagttctgt tccatacatc cctttcacct |
| 71521 | ccacggcgtt gtagcatcct gctttcatga ctgtgtcatc actcggaagg aactgcttct |
| 71581 | cttccagaat gcttttcaag atctactctg accacagcta taaactttac acttctattc |
| 71641 | tcttcttgcc cctcacagtg ttctctgttc ctctaagatc ttaaactctg tctactccta |
| 71701 | atccagcctg ctgggtgtgg ctggagaaag tcccactggg gggctgatta gttaggaatg |
| 71761 | tagggtttcc agctcttgct ggagcctcag aagagttcag cagacttttt tttttttttt |
| 71821 | tttccttaaa cctatttctg cagccttgat gaccactcct tccagtccct cacctatttg |
| 71881 | ctttattcat ggcagaggct ctttcttcct gcttgtcagt acaaagaggc aggattcttc |
| 71941 | acctggatct gtggattctc aaagaatttg tggagagaat tcagggcatt gatgaccttg |
| 72001 | gatgaagaga aatttacatc tttatttaca ctaaccttca agtgaaattt agcatttttt |
| 72061 | gccatttaaa aatatgggca acaaacaact agtagtatta gcagtattta tgacttaagc |
| 72121 | acctatagaa ctcagttaat ttcatatcgc ttgatgttat gggtatctca aattattatt |
| 72181 | ttatgtatat atatttttga gatggagtct cgctctgtct cccaggctga gtgcagtggt |
| 72241 | gcagtctcag cccattgcaa cctctgcctc ctgggttcaa acgattctcc tgcctcagcc |
| 72301 | tcctgagtag ctgggattac aggcgcacac caccacgcct agctaatgtt tgtattttca |
| 72361 | gtagagaagg ggtttcacca tattggccag gctggtcacc aactcctgac ctcaagtgat |
| 72421 | ccgcctgcct tggcttccaa agtgctggga ttacaggtgt gagccaccgc acccggcctc |
| 72481 | aaattatttt tagaaacaga atcttgatat ggtatccgct ctggccttga acttgtgggc |
| 72541 | tcaggcagtc ctcccacctc agcctcctga gtagctggga ttataggcat gtgccactgc |
| 72601 | accaggcttc aaattattat gtatgttcat cacctcttta aatttataat agttattaaa |
| 72661 | cctgttactg gatcttaata tttaatgctt taattaagaa catgtatgct actatgccaa |
| 72721 | cagatttttt tagtttttga taactgcatt tcattgttac ttgttctcat ttgatttcct |
| 72781 | gtgtatttta cgaatttaag tacattctga atacggtttc ataggcttcc ctaaaatatt |
| 72841 | gaaggggccc atggattaag aaaaaggcta agaatcccta atctagaggc tccccacagt |
| 72901 | cctcttttgt catcataccc ctaccccatt ctagcctgag gagcgtggct ccacctgtgc |
| 72961 | ccttggtttt gttgttccag tccatacatc ctgcaccctt aactgtgttt cttatcccca |
| 73021 | acttgtttct ttgtgttatt cttcagtatt atagtcttta atataatctg tataatacat |
| 73081 | ggtgtagtag tatatgctcg tagtatacaa ttcagttaga acagatgagt attcaatgaa |
| 73141 | aagataatct cctctctaac ccccagtccc acttccctgg ggaagcctgt gttcttgtgt |
| 73201 | acaattcaga aaatgtttat acacatattt tttatttatt tattttttga gacggagtct |
| 73261 | cgctctcgcc aggttggagt gcagtggcgc aatcttggct cactacaacc tccgcctccc |
| 73321 | tagtagttca agcaattcaa ggttcaagca attcgcctgc ctcagcctcc cgagtagctg |
| 73381 | ggactatagg cgtgtaccac cacgcctacc taatttttgt atttttagta gagacagggt |
| 73441 | ttcaccatgt tggccaggat ggtctcgatc tcttgacctc atgatccacc cgcctcagcc |
| 73501 | tcccaaagtg ctgggattac agatgtgagc cactgtgccc agcctgttga tttaatttta |
| 73561 | aacagagttt cgctcttgtt acccaggctg gagtgcaatg gtgcgatctc ggctcaccgc |
| 73621 | agcctctgcc tcccaggttc aagtgattct cctgcttcag cctcccgagc agctgggatt |
| 73681 | acaggcatgc accaccatgc acagctatat ttagtagaga tgggggtttc tccatgttgg |
| 73741 | tcaggctggt ctcgaactcc ggacctcagg tgatccgccc gcctcggcct cccaaagtga |
| 73801 | tgggattaca ggcgtcagcc actgcacccc gcctatacac atttttttgt tttttgtttt |
| 73861 | tttgagatgg agtctcgctc tgttgtccag gctggagtgc agtggcgcga tctctgctca |
| 73921 | ctgcaagctc tgcctccctg gttcacacca ttctcctgcc tcagcctccc gagtagctgg |
| 73981 | gattacaggc gccggccact acgcccatct aactttttgt atttttagta gagatggggt |
| 74041 | ttcaccgtgt taaccaggat ggtcttgatc tcctgacctc gtgatctgcc tgactgggcc |
| 74101 | tcccaaaatg ctgagattac aggcgtgagc caccgctccc agctatacac gtatttttaa |
| 74161 | tgccactcca gtctatgttg gaaccatttt acttcccctt tcttattttc ttcttgtgtt |
| 74221 | cttgaaggcc tagatcagct gttgctgata ggctgtcact gtcactttag aaagcccaga |
| 74281 | gccttttgtt ccttagaact ttgtttttaa ttgtattgta gcactcattg tattcgattc |
| 74341 | taaaagattt gcttcatttc tgtaactagt ctcttacacc caggagctcc tagttcctac |
| 74401 | aggaaatgct gggaattgta tcagtcaaat gtgaatcccc acctcgtcca gacttatgag |
| 74461 | tgcattgtag gtactcagta agtgctaaaa atgactaaat agtcccactg ataccaatct |
| 74521 | atatactgat actttatata gtatatagat tggtccacat ataacgatga cacataatga |
| 74581 | gaaactgtct taaaaagttg ttgaaagtgc cgcaggaata ggaattgatc aaaacaatat |
| 74641 | gattttttag gtttatatgg aactttgatg tttgagaaaa ggctgattta gttgagaaga |
| 74701 | aatggttagc tgaggatttt gatgacttct ctggaagcac atttgagggt ttgtgatgtt |
| 74761 | aaatctgatg ttaatgatta tttcatccag ttttatgtca ttttatagtt tttatacatt |
| 74821 | taagtatatt tatttctaat gtttaacact accattttag ttatttgacc attattctgg |
| 74881 | ccctttaaaa aatgctcaga caagtttgaa tgatttttca gaggcattat tggctcagag |
| 74941 | gtaaaagagg aaagattgag aagctgaata tgtactctgt ttcctgggta tggggctggg |
| 75001 | gatacccaga agaggttcac acgttggtcg agacatttct ttatgaccac cagcaggtgg |
| 75061 | catcaccggc ccaaaatgac taagtttctg cccagaatca gaagagaagg tgttgagagc |
| 75121 | ccactgctgt gggggtagca tggaggtggg atacaggggc tggaggtgat acaattttgt |
| 75181 | ttcttcctcc aacatcgcct gctagtctag aggcttttat aaattgaaaa actaattctt |
| 75241 | tatcatctca tctgatggtt tttatgtttt tccttttttc tctctatacc tgtagttcct |
| 75301 | tcagaaacag gtaacacttt tctaatagtc acgttgtatt cttgcatctt gttgttacaa |
| 75361 | tgcttttgtt tctcaccata ggggatgatg gaaaattaat attctttgac ttatggcatt |
| 75421 | ggtaaaatct gcatgcaaat tcccacagtt gcctgtagat tagagccagt tgtttttttc |
| 75481 | tcaactttgc aggaatcctg gttacaacat tgtactattt actaccaaca gtgttttttt |
| 75541 | tttttaaaat ccagacttgc tgggcatagt ggctcatgcc tgtaatctca gcgacttggg |
| 75601 | aggctgaggt gggaggattg cttgagccca gggctgcagt gattgcggca ctacactcca |
| 75661 | gcatgagtga caaagacccc atctctgaaa aaacaaaaac aaaaacaaat tttttttaaa |
| 75721 | gaaacagaaa caaaaatcca aacttgtaac cactgtaaaa caaatcagaa tttacgatag |
| 75781 | tggatattat taatagtgca gaatggatac ccagatcttg cttcctttct agctaatgat |
| 75841 | gcaatgttgg cctgaaatgc attacttata gccagggatt ttctcagcat cctgatgata |
| 75901 | tagcctcatt tcgtgctaac tctccacttc tgcacatctt cccctaagtc ctttactcat |
| 75961 | ctttagaaag agctactttt ggtgaaattt taaaaccaag gaatatcatt ctttatagaa |
| 76021 | tcacacttct gtgttttccc cttccccatt tctgtctcga aagcgacaga ctgctacata |
| 76081 | acctgtgaat actttttttt aaaaaaagtt tggtattgta aacagaagat ttaagattaa |
| 76141 | aatgtagcat tgagaaaaat agatttatta ataatgccct cttaacacaa cctaaattct |
| 76201 | ggtcagtgga ataaagcctg ggtcctaaag ttttagacgc ttgcttgctt ttccacactg |
| 76261 | gctcttactt ggggatcctt ttagaaattt gtttagaata atactgtaaa aacatattta |
| 76321 | agctactttg tgtgtacatt tgggatcttt tggtttgaag acggcttgac tcaagacttt |
| 76381 | ctaaatattt tcacacacac acacataccc tgtagtgaga aaaaaatccg tttatatggt |
| 76441 | tctataaaaa tctctagctg cttcgagctt taatttcttg aatcaaaaga gtattgtttt |
| 76501 | taatactgag cttctatcta aataaatgct ttatttactt aaatgtgtgc ttttcaaaaa |
| 76561 | ctagtatgat taagacatta acaggatctt agacgtaaag gaacagtcct gttgcttctt |
| 76621 | ccagaagata atatgactcg tttggaattt tcctatagtg tagttttttg tctagtgttg |
| 76681 | tgagaattaa agggatttca ggatcttaag gtaggttatt atttgatgct ttcttggaac |
| 76741 | attttacatt cttgaaaata cacatggcta aattaatttt tgccagcaat ccacataact |
| 76801 | ttaagataat gtagagaaga acgtgattca ggttagtatc aaataaggtc agatttctag |
| 76861 | tgccatcagt agctttcagc aaagatgagg tgctggtaag atagcattag tctcttagaa |
| 76921 | tctcttagag agattttcca aaattcagcc atttctagtg aatgctccat tccaccccca |
| 76981 | gctgagtcct gctgctctgg ggaactccct cagcacactc ttggctctta gaattgctag |
| 77041 | caatgggagt agtgctgctg gtggagctgg cagctaagcc cagaggtgga ttaatgcttt |
| 77101 | tattccctga tgtacaggta cacacactca tacctaccca cacctagttt gggataagaa |
| 77161 | gaggttagaa ttagctaggc ttgaagttcc atgcttaaat ttgctggctc agatttctta |
| 77221 | ttttggcatc actttgccca ttagggagac aatgacagtt atagaagcat tgccaaataa |
| 77281 | aaaatccatc tggaataacc tcttttgtag gagtattgtg tgtttagttg ttgattcgtc |
| 77341 | ccttcctcct cttagtggca acttacagta ctgggaagga acagtggctg ggagcttata |
| 77401 | ttcctcagca gagccagatc agcagaagta ttactcctta gttcgtagta ggtggtaccc |
| 77461 | tatgggtcca gtcatttaaa tgcaagcctg tacctacaga gcgtttccta gtgccatcat |
| 77521 | tgcccagtgg gcctttattt agctgagtct aactcccaac tagagaaaat ttcctgtgcc |
| 77581 | agacagcagt atggtcagct aacatgtgga tgctacattt gctttcataa gtcagtactc |
| 77641 | ttcaataaca ttagtagaag agaagaggac acaaagtgag agtgtgttaa taggaagtcc |
| 77701 | aggtatgcct gctacctgaa ctttctgaga caggtaatac tgtagggcct gaactttgta |
| 77761 | gcagagtggt tatatatgaa gaagtgggtt ctgggagggg ttaaaccact tagaatggct |
| 77821 | tcatttacta atggcaagag tttgctggga tattgaccac tgtacataga catgaatatg |
| 77881 | gaaagttaaa aacaaaatcc acatatattt ggctgcaagt actccgaagg tatatctaat |
| 77941 | tagtgcatcc attaaacaaa agagatattt taggccgggc atggttgctc acacctgtaa |
| 78001 | tcccagcact ttgggaggcc aaggtgggtg gatcacctga ggtcaggagt tcgagaccag |
| 78061 | cctggccaac atggtgaaac cctgtctctg ctaaaaatac aaacattagc tgggcgtgtt |
| 78121 | ggtgggcgcc tgtaatctta gctacttggg aggctgaggc aggagattcc cttgaacctg |
| 78181 | gaaggtggat gttgcaggga gccgagatgg tgtcactgca ctccggtctg ggtgaaagag |
| 78241 | caagctccat ctcaaaaaag aaaaaaaaaa aaagagatat ttttgatgga ttgatagaaa |
| 78301 | ttttcttttt cttttttttt ttgagacagg gtctcactct gtcgccaggc tggagcacag |
| 78361 | tggcgtgatc tccattcatt gcaacctcca ccccccgggt tcaaacgatt ctccttcctc |
| 78421 | agcctcccga gtagctggga ctacaggcat gtgccaccat gcccaactaa tttttgtatt |
| 78481 | tttagtagag agagggtttc accatgttgg ccaggatggt ctcgatctct taacctcatg |
| 78541 | atccacctgc ctgggcctcc caaagtgctg gtattacagg catgagccac cacatctggc |
| 78601 | cagaaatttt cttggtcact tctgagacat gcagagtaat tacctgtaat ataatttaat |
| 78661 | gaattatgtc aatatattaa aatatgcttc atgtgggctg ggcatggtgg ctcatgcctg |
| 78721 | taatcccagc actttgggag gccaaggtgg gggtatcact aggtcaggag atcaagacca |
| 78781 | gcctggctaa cacggtgaaa ccccgtctac taaaaataca aaaaattatc cgggcgtggt |
| 78841 | ggtacacacc tgtagtccca gctactcggg agactgaggc aggagaatcg cttgaacccg |
| 78901 | ggaggcagag gttgcagtga gccgagatca cgccactgca ttccagcctg ggcaacagaa |
| 78961 | cgagactcta tctcaaaaaa aaaaaaaaaa tgcttcgtgt ggcttaaaat tatatgaaaa |
| 79021 | gaaaatacct ttactgatag tcatctgtga ttccatttgc taaattaaac gtgaaagcat |
| 79081 | acttttactg aatactatat attccgtatc agcttagata gcagtttatc ttcacataca |
| 79141 | taagttttaa gtttaccttt attatagtgc attggtcttt tgttttcatc aacctaaatt |
| 79201 | atgttcaata aatgtttctg ttagatttta agttaaacaa ttatgtgaaa ttcatttttc |
| 79261 | gtaattgttt tttaacatat gtctttgttg gtaattcacg tgtgtgagtg taactgattg |
| 79321 | ccagattata taaactttca accaaaacca ttctttgcag atgcttttac tgactctgct |
| 79381 | atcagtgcta aagtgaatgg cgaacacaaa gagaaggacc tggagccctg ggatgcaggt |
| 79441 | gaactcacag ccaatgagga acttgaggct ttggaaaatg acgtagtaag taacatcttt |
| 79501 | gtaattattg ctagactctg gtcagtatga catcctgtca cttggttgta atttaaatgt |
| 79561 | gcttttgttg ttgttgttat tgtagtgagt gtatttagag cagcaggtct gttgtataac |
| 79621 | tagagacttt ctcccaagca atatataaag aaaaatgttt gtcattttac ttgtaggggt |
| 79681 | taagcaggag tactgtctgt tcttgtggat gctcatgaat tacttctttg tgattaaaat |
| 79741 | aaataataag aagtagctta aattaaaatt agaaaccatg ggaaatgccg gtgtgttttg |
| 79801 | ctttaacacc cagccaaata aggtagccta aggaaagtgg tgtcttaatt gttgacttca |
| 79861 | cctagagaag aggttgaagt aggacatttt aagcctcttg tctgaagaaa aggttgtcat |
| 79921 | taagataaat aattaggtta cattggaatt aaagcattac ataaatttct tggtcttaaa |
| 79981 | tttggattat tctccacaaa attcttttat ttctaaaacg cctcttgtca catactagtt |
| 80041 | ttgtttctct ctttaatgca ttatctgtac ttgaagtgct tagctgggta tgctggcaca |
| 80101 | tgcctgcagt cccagctact tgggaggctg aagcaggagg atcacttgag cccaggagtt |
| 80161 | ggagtccagc ctgaatgaca taaggagacc ccttctctaa gaaataaaaa taaaaacaaa |
| 80221 | tacttaataa agactctgtc tttaggatag agagcataga gatataaagc aaagtgtctt |
| 80281 | gccaaaaatg agtgttatgg taccaatatt tgagtagaat gaagaatctt ccattgagta |
| 80341 | gaaagagaat ttgtaacata tctgtgtttg atgtttaagg cataacagct taataatgac |
| 80401 | actcttcctc agacaggaag cctgaaatgt cctactttga cctaaagtct agtaataaaa |
| 80461 | ctggacatac acaggcaaca tgtcattaat tctcaaactt taacaaatca tatataacct |
| 80521 | aatataatgg ttctcaagtc tgtacatcac gtcacctgta tgaaaaatat gaggaaacag |
| 80581 | agacttcttt tacactattg gtgaggtgga taaattgata gagtctttct ggagagaatc |
| 80641 | tggcaatgct aatcaaaatt taaaatgcac atacactttg ttccagcagt tctatctcta |
| 80701 | gtaatttatt tttgccctca tatatccata agacatgcaa ataattatat gtgaagattt |
| 80761 | tttttttttc tttttctgca gagacagggt tttaccatgt tgcccagggt gatctggaac |
| 80821 | tcctgagctc aggtaatcca cccacctcag ccccccaaag tgctgggatt acaggtgtga |
| 80881 | gccatcatgc ctgaccagga tttttttttt tttcagcatt atttcttttg ttgttgttgc |
| 80941 | tgttgttttg agagatggag tctcactctg tcacccagac tggagtgcag tggtgcgatc |
| 81001 | tcggctccct gtaacctcca cctcctgggt tcaagtgatt ctactgcctc agctttccaa |
| 81061 | gcagctggga ctataggcgt gcgccaccac acccagctaa tttttgtatt tttagtagag |
| 81121 | acggggtttc accatatgtt ggccaggctg gtcttgaact cctgacctca ggtgatctgc |
| 81181 | ccacctcggc ctcccaaagt gctgagatta taggcgtgaa ccaccatgcc tggccatagc |
| 81241 | attatttcta atagtgaaaa attggaaaca tgctaagtgt ctatcaatat agcatgagtt |
| 81301 | agatttatga tgtcaccatt caattgaaac actacatatc tcccaaaaag aatggtgttc |
| 81361 | caatatggaa agatatctaa gatttattaa gagaaaaagc acattgcaga acactgggat |
| 81421 | cctatttgct tttttttttc tttttttgag acagagtctt gctctgtcac actgcaacct |
| 81481 | ccgcctcccg ggttcaagcg attctcctgc ctcagcctcc tgagtagctg ccaccatgcc |
| 81541 | cagctaattt ttgtgttttt agtagagaag gggtttcacc atgtttgtca ggctggtctt |
| 81601 | gaactcctga actcgtgatc cacctgcctc agcctcccaa agtgctgcga ttactggcat |
| 81661 | gagccaccgc acctggccat gaaatttttt ttttttttta aagagctgtt catattctta |
| 81721 | ttgcctagaa gatgtctgaa attacaccca agaaactctt tttgagacgg agtcttgctc |
| 81781 | tgttgtccag gctggagtgc aatggcgtga tcttggctca ctgaaacccc tgccttccag |
| 81841 | gttcaagcga ttctcctgct tcagccttct gagtagctgg gactacaagc gcccgccacc |
| 81901 | acatctggct aattttttgt atttttagta gagacagggt ttcaacatgt tggccaggct |
| 81961 | ggtcccgaac tcctaatctc aggtgatcca cccaccttgg cctctcaaag tgctgggatt |
| 82021 | acaggcatga gccactgcgc ccggctgaaa ctcttttttt ttcttttaag atggagtctc |
| 82081 | gctctgtcgc ccagacttga gtgcagtggt gtgatctcag ctcactgcaa gctctgcctc |
| 82141 | ccgggttcac accattctcc tgccctagcc tcccaagtag ctgggactac aggctcccgc |
| 82201 | caccacacct ggctaatttt ttgtattttt agtagagaca gggtttcacc atgttagcca |
| 82261 | gcatggtctc aatctcctga cttcgtgatc ctcctgcctc ggcctcccaa agtgctggga |
| 82321 | taccaggcat gagccaccgt gcccggccag aactcttaat agtagttact tatgcacgct |
| 82381 | gggattggaa gacatttact ttttactgga tgtctttccg tattgtgtgc tttttttttt |
| 82441 | ttttttttat gtagggcata cattacttaa gtaattttaa agcctccata agtaagtgtg |
| 82501 | atttcctgcc catgtgtttg gcaaaaggaa ttgcattggt ggtagactta cattatagtc |
| 82561 | ttacctggag tagcacagga ggacccaagg ttaataggtg aacttcgagg caagccttag |
| 82621 | cattgaggtt gccatcagca ttgcttggtt gatgtgttca ttcttctggg atggattaca |
| 82681 | acctttactg gactttatac ttttcaccag taaggcttta aaaaaggagt tgaaacatta |
| 82741 | gagaataatt atccaggcag taatattcac tggtaaatag tcttccagcc tgtggcccaa |
| 82801 | ttggttgatt cttttacgtt aaagaatgca gcctcagctg ctctgcctat ggagtaggat |
| 82861 | tcttttattt actttcttaa taaacttgct tgcccctggc tcccccccac caaaaaaaga |
| 82921 | aggcagcctc ccttttgcga atggtaattt cctatagttt cctcgtagaa ttgtggagtt |
| 82981 | acctatgctg aggttatagg ttagggtatt gagatccaga gttgccactt ctgaggtgtc |
| 83041 | acaactgcta atggtaaaac catttctaaa gcccagttct tgtgactttg tccagtgatt |
| 83101 | gcctgttcac cgtttcatgc tgccttccca tttgagcatt cccaggagga aggggaggtt |
| 83161 | gccagggacc tagtaccata gtccgacctt ggaatcgttg aatatgaggg aaagcgttgg |
| 83221 | cttctccctt ctttctccca aacattggaa gtatttttgg ctgttaaaaa gcaccccttg |
| 83281 | ttccatgtgg aatcccttgt ttaaaagaag taaaatatgt acctcctgtc ctccacagac |
| 83341 | ctgaggacca gtgtgatctc aagaaggtta caggtaaatg tagatgtctc taactgaaag |
| 83401 | gtggctttta caggttagag aaaagagaga accctgatct gaaggctatt ttatgaagta |
| 83461 | attaaaatgt tctaaacttt aaaaataact gctcaaataa ttgtgttgta tagttactta |
| 83521 | tcaactggag gggctgataa gtatttttct aaaacatttt taaggaaatt ttttcctatt |
| 83581 | ttctaatttg ctaattttgc tcaagtagtt tgttagatat tgttaatata gatgttggtt |
| 83641 | ataactgaat gaaagggaac aactactttg acattttgaa aaacaagctt cattttcttc |
| 83701 | tagtctaatg gatgggatcc caatgatatg ttccgatata atgaagaaaa ttatggtgta |
| 83761 | gtgtctacgt atgatagcag tttatcttcg tatacgtaag tttgaaaagt ttgtttttat |
| 83821 | tttagtgcat ttgtctttga ttttcatcag cttaatttat gatgaataaa tgtttgttag |
| 83881 | tttttaagtt aaacaattac atgaaataat ttttctctta ttaccaactg tgataaattt |
| 83941 | ccattaaaaa aagggaataa atgtagtttg cctataccct gtttttatgc tctaaacaaa |
| 84001 | ttttggtttt gtcttttttt ttcttttgag agggaatctc gctgtgtctc caggctggag |
| 84061 | tgcagtggtg caatctcggc tcactgcaac ctctgcatcc cgggttcaag cgattctcct |
| 84121 | gcctcagcct cccgagtagc tgggactata ggcgcgtgct accatgccca tctaatttct |
| 84181 | gtatttttag tagagacggg gtttcaccat gttggccagg atagtctcga tctcttcacc |
| 84241 | tcgtgatcca cctgcctcgg cctcccaaag tgctgggatt acaggtgtga gccactgtgc |
| 84301 | ctggccggtt ttgtcttcta agttgttaaa aaatatctaa atttgcaagg gcagagatta |
| 84361 | tggtgaacag tttaaccagt ttttgaaata tgctcctctg gagaaaaggt aacagaaaaa |
| 84421 | aaagttagaa ttttgattta taaatacaca gatcactata acttttagct ttagttttag |
| 84481 | ttttagtttc tgtttttacc agtattctaa actctaaact ttcttagtag ttgattatga |
| 84541 | cagatacata aactgtggct ttaaaggact cattttgctt ttcttttcct catgtttcag |
| 84601 | agtgccctta gaaagagata actcagaaga atttttaaaa cgggaagcaa gggcaaacca |
| 84661 | gttagcagaa gaaattgagt caagtgccca gtacaaagct cgagtggccc tggaaaatga |
| 84721 | tgataggagt gaggaagaaa aatacacagc agttcagaga aattccagtg aacgtgaggg |
| 84781 | gcacagcata aacactaggt atttaaagga aatcatgatg cagtatttag gatacacaac |
| 84841 | tcaaggtctg tgtgagacgg tgtattgtta ttatatttcc tcttccttta atatagctta |
| 84901 | ggtagagaat gcaagtagaa ttggtttaag atctgttaga gaaaaggtta tggtgatctt |
| 84961 | ggaaaatatg cttttgagag taagctctgt ggagccaagt gttggtatat cacggtgagc |
| 85021 | aatccaagat cttgaagagc ttgttaaaat agttatctgg tgggggacac gtgtaacaat |
| 85081 | cacagcagta caatatgatt tgcttggtta aaggcatgtt caaagtacta ggaacataca |
| 85141 | gaatgaggag gagctagcat aacctgtaga gtcagagaaa acctcattga ggaggtgaca |
| 85201 | ttttgtgata agataatagg gtctttgaca cttagagaag agttgggaga agagtttatc |
| 85261 | acctgatgaa aagccatgta caagcatggc tatgagaaaa tttggccagc tcaggagagg |
| 85321 | gctggttgtt gcatgtgtct ggaacacagg atctgtgtca ggtgcagcag tggcagttga |
| 85381 | tagtaggaac tgaggtcatt aaaggacttg gcatgtcatg ctaaagagca ccctgttgga |
| 85441 | aggagatggg gtgaataaac cctggggcat tgaggactgg ctgagacaca gagaacagtt |
| 85501 | agtgcactga aatagttcaa ctgtgagaat ttggtaacca cctagttaag ggatgagcct |
| 85561 | gaggtttatt tgataactaa gtgacttaat ggatgtactg gtaagagaga gaggaaacat |
| 85621 | ggagcaagtt tgaggggaaa aacagtgact ccgtttgtgc agctaattgc atatgtgggc |
| 85681 | ttgtgggtct ttcatttatt cataaacgtg ttgagaaata cctgctacct atctagtaaa |
| 85741 | gtaagagatg catcctctct taaaggcagt cagcttagag tctggtgatt tgaattgaca |
| 85801 | tgtccactga tagatgttga cactgtgaga ctggcggttc agtttgaggt ttcatcagca |
| 85861 | ttgccgatat tggagccatg aaaaaccaaa gaacagccag tgagagaaga gatctcagag |
| 85921 | aaaataaaat tgagaaagtg aaggacaaaa aatgttgtga agatagacca agattgatgg |
| 85981 | aatcagccat agagaggtca agtgggatga gaatgagcac gcatctgtta aactttgtgc |
| 86041 | ttaggagcag aatctaaggg aagggacagt ccagaggtta gaactcaggg taagatggaa |
| 86101 | gaacaggggc atctgggagt gaggcagttt ggcttagtgt agaacctttt tgtaacaagc |
| 86161 | attcccttct gtctagatga cttttagata tgtttcattg gcttggtacc ttttagaata |
| 86221 | aaatgattta gaggatctct cattttcagg gaaaataaat atattcctcc tggacaaaga |
| 86281 | aatagagaag tcatatcctg gggaagtggg agacagaatt caccgcgtat gggccagcct |
| 86341 | ggatcgggct ccatgccatc aagatccact tctcacactt cagatttcaa cccgaattct |
| 86401 | ggttcagacc aaagagtagt taatggaggc aagtattttg accagacttg tcaatatcat |
| 86461 | tgataaaata gttttctaaa tacttaaaat acttaaaata gtttacataa ctgatatgaa |
| 86521 | tgtgcacttt aatgatttgg tgagtagctt tcacttcagc attacttaaa attggctttt |
| 86581 | gtggatatta aattagtaaa acattgtata tgtcattgac atatatatta tttagcatga |
| 86641 | tgaaatattc atgatgtact aagataaagt gctacattta acccaagaca atcacttggc |
| 86701 | caaaaacact tcacatataa agaaattgga aactttgggt aggttctcaa ttttaaaaac |
| 86761 | actggataat aaaatttttt agacataatt tatatggaaa attctaacct atgtgcaaca |
| 86821 | ctgtggttaa tatagatcaa ttttcattat ttgtttctat attatgctta cttcaagaaa |
| 86881 | ggatctgagg taacttataa tacaagacat gatcaagagt catgtgaaga aagtgactag |
| 86941 | agaaatttgc ttaaaaaaca acaaaaacaa cccttagtct aagggtggat gttacagttt |
| 87001 | agcaacttaa gtaaaagaaa cctgaatctt tagtaggaag acatttttta ctctacctct |
| 87061 | aaatctaggt tgaatatatc ttgtaggttg tggatctttt ccataaatca gggatactga |
| 87121 | acaacagttc tatggatggt atggaaatag taatagcaat agtatgttac taactttgtg |
| 87181 | ggaaaagagt ggacattcaa ttttagctat ttaaatttgg aaagttagat gaaaatagag |
| 87241 | aacactaagt ttccaatttc atttgttttc attgagtctt ttctccagaa ttcctctcca |
| 87301 | aatggacact cttgagtatt ttcagtactt aatattgggg gtgaaatttc tttgctcact |
| 87361 | gaggaaagat tttagttgtt tataaacaga attttaaagt taaaaaacct gaagggggct |
| 87421 | gagaaatata tgatacttaa gtgtgtggaa ccctatggag aggagacctg gactgtttga |
| 87481 | taagattaag gtaagtgata tgtaatgtta aatactagct gtatctttac ctaggcatat |
| 87541 | ccatcagtat aaatttattt ggtgatgact gctttgtagt tgcagtattt attaagcagt |
| 87601 | cgcttagata agtgtttaac tgtataaatt atttagaagg tctcccttct tctagtttaa |
| 87661 | tgaggtcaag actttttttt tgaaatagca atgaatatta tcatttgata ctcacaggag |
| 87721 | tcacaaactc tagaagagta atgttttatt tctacttaaa tgggacttgc ttaataagat |
| 87781 | tccaaactga gttctgggtt caagtgtaaa cctgatgaaa atcatagata attgtaagga |
| 87841 | accagcattt ctaattggat ataatagcta ctgcttattt tcgttatgcc tcagagttaa |
| 87901 | aactaataca gtaaataatc ttactcctga gtaggaatta ttgtgattta ttatgtgaaa |
| 87961 | ttatctagtg tatgttatat tcctttaaac aaccagttac tgagaaacag ttatagaagc |
| 88021 | aggattaata ggcaaagtct taactgtctt cttcaatagt gtgtatagat cctaattaac |
| 88081 | cctttgggaa cgtgtattca tttaaacaga cttaatctta aggaggttaa agtaaaatgt |
| 88141 | gaatttatgt cagttaagtt atgctaaaac ttatcacaaa tcaaatgact gtcctcaaag |
| 88201 | ggttaaaatg tacaagaaat catttttgtc attttacttt ttttctgttt acttttttcc |
| 88261 | ctcatttttt tctttagttt ttatactttc cttcatatca tttgttctgt caggtgttcc |
| 88321 | ctggccatcg ccttgcccat ctccttcctc tcgcccacct tctcgctacc agtcaggtcc |
| 88381 | caactctctt ccacctcggg cagccacccc tacacggccg ccctccaggc ccccctcgcg |
| 88441 | gccatccaga cccccgtctc acccctctgc tcatggttct ccagctcctg tctctactat |
| 88501 | gcctaaacgc atgtcttcag aaggtacaat accacaattt gttcatgttt ttgtttgtct |
| 88561 | ttgtttaact cctatgtgag tttataatta caaaatagtt tcctcttcat tatttaataa |
| 88621 | cctataattt ctgtgtttta actttagttt attaaaacta tttctattaa ccttttgttc |
| 88681 | attagagaga aatttgataa atgtgtgaag ctataaactc tcttgaatcg ttgttaaaaa |
| 88741 | gggggtttat ctctgcctga taattatgct tctttacagc cccagaaggg tctgccccac |
| 88801 | agccttcccc ctccttattt gcactgtata cagtagttaa acaaatgaac tttcttcagc |
| 88861 | cagtcttgaa cttaggttca ttttacagct ctctggccaa ggtcctagtg aaccttccta |
| 88921 | ttggccataa gcagggatgg tgttttctgg gtcttttttg agagcgacag cccatgtagc |
| 88981 | tgactttgcg tgtctgccct tagattaaag tagttgattt ttagaatgcc agaagaattc |
| 89041 | taaatttaac tgagtaattt ttttaaagtt agctttgcaa tcttacatag tgaaaggctg |
| 89101 | ctttaatctg gaagaagtcc ttgatctgag ataaaattga taaaaacgac atatgaattt |
| 89161 | gaatatttag ctatttcttt cctcgtcaaa aataagaata aaatcttgta attcttattc |
| 89221 | agtatttggc gctaaatcca tcattgccac atatcaaata cagggatatg ttgtagaaag |
| 89281 | gtaacattct aatttaaatg ccacccatat attaaaaacc tgttttctga atcataatgt |
| 89341 | ccttttgata ctagttctga atatttgtgt taaaatttta atctgatttg ttcattaaaa |
| 89401 | ttagttaata ttgcttatgt tgggactaat aaagttttcc gcacaaaatg tgtttctcct |
| 89461 | gcttccctgg agaaaactgt attggctact tttaaataaa ttgttaccat ctaagcaggc |
| 89521 | aggtcatatg actttgactg aagcatctaa ccttgaagag caagttccac tgattttcaa |
| 89581 | ggtgacttct ttgctcaaaa gggccttaat agtggtcact aaatgcaaaa ttctgttgat |
| 89641 | atttttcttg tagtccatca tttgagtaag cgatgtttat ttaatgagaa tatattaaat |
| 89701 | aaaacatgat cattaatgac tgtgaacatc tttattacat taagatttaa ggactgctca |
| 89761 | tgtattaact tcacacagaa atatactttc tgtgtcattc agagatgttg aatatttcca |
| 89821 | tttgaaaatt atagtgtata acattagcat tcttctaaag atcatgttcg tgtttaaatt |
| 89881 | cctgttggaa gccaggcatg gtggctaacg cctgtaatct cagcactttg ggaggctgag |
| 89941 | gcaggtggat cacttgaggt caggagtttg agaccagcct ggccaacatg gtgaaacctc |
| 90001 | gtctctacta aaaataccca gctacttggg aggctgaggc aggagaatca cttgaacctg |
| 90061 | ggaggcagag gttgcagtga gttgagatcg taccactgca ctccagcctg ggcgacagag |
| 90121 | acagactctg tcttataaaa ataaaaataa aataataatt ctattggcaa catatattaa |
| 90181 | tttgaagttc taaagagttt ggcagccggg tgagagagtg aggagatttg gctttgacat |
| 90241 | tagggaagtt ttcgcttggt gttaacacca gtaggcttct ctgatgaggg ccattctgtc |
| 90301 | cactctttta cctgatagat tggtctaatg cacagtagac tgatttagaa agagtagtca |
| 90361 | ctagtggcat ggcagaatca ataatgtaga attttgacaa ttcatatagt gctgatttct |
| 90421 | cccccaaatg tcagttattt tggtcatcta ttaatagact aatacaagtc atccctttaa |
| 90481 | tagaattttc agctcacagc ctgctaagcc taagaaactg cttacaggct actgcttact |
| 90541 | gttttaagcc gagttttaaa attgatgatc atgatagaag agataaataa actaaaattt |
| 90601 | tagagaaatt taagaagggt atgtacatat gttttagtgg tatcggggtg tatagggatt |
| 90661 | aatagtcttc tgtttaaatt ttttttttct aattttagaa gtaatgtaga aaattcgggt |
| 90721 | cagggaaagg taaaatatat ggaaagttaa aaatatttta tcatgtagtc ataatttcta |
| 90781 | gtaacatatt tctttacaaa taagacatag ttgaaacaga ttgctacagt tcttttaaga |
| 90841 | gttgacatct tattgttgat ttcttaccac caacttcatc cctccctttc tttaaaaata |
| 90901 | aagggaaata ataaaattta tttataaaac tttgtggcat tccacaaaat aattctgaaa |
| 90961 | gaattagtat ggccaaaaaa atatgtatgg tgtttttttt ttttctattt ttaaccaagg |
| 91021 | aaaaactgta gagtgagtga gtgtgtgtgc atgtgtgtgt gaatgggtgt atttagcaga |
| 91081 | aaagtagtac tgatgaatat catggaattt atgtgatgtt cactgtttct tccttagggc |
| 91141 | ctccaaggat gtccccaaag gcccagcgac atcctcgaaa tcacagagct tctgctggga |
| 91201 | ggggttccat atccagtggc ctagaatttg tatcccacaa cccacccagt gaagcagcta |
| 91261 | ctcctccagt agcaaggacc agtccctcgg ggggaacgtg gtcatcagtg gtcagtgggg |
| 91321 | gtaggtaaca cttgggcata atgatggtac tcattttgtc attacactag atataaagag |
| 91381 | ggctgagcta caactctgtt tgaggaagtg taagtatgta tatgttaaaa atagtagaat |
| 91441 | caccaggaat tgggaaaccc atatttttat tctgggctct accacttatt catcatatat |
| 91501 | taaagcaagt cagacactca ttctgaagtt gagatttcgc agtgagtaaa gtgttaataa |
| 91561 | ttcttgccta gtctacatta tgggattgtg atgagattcc tataaggttc ataaatacag |
| 91621 | atatattgta aaactataaa gttttgtaaa gtacctctct aatatgaggc aaacacagta |
| 91681 | tgtaacacta tttggaggga ccgtatttcc ttatcttttt agcagcttcg tttatcagta |
| 91741 | cattctataa acatttattt ttggcttaca ttgtagtgtg tttctatagc atctgtatat |
| 91801 | ggcactaatt cccaactata tttccataat aaggaatatc aaatacaaat aaagggtcca |
| 91861 | agttttattt gtgattagca taaggaatat gctgacagca gctataaaag tataaaaatt |
| 91921 | aggctgggtg tggtggctca cgcctgtaat cccagcactt tgggaggctg aggtgggcgg |
| 91981 | atcacaaggt caggagatcg agaccatcct ggctaacacg gtgaaacccc gtccctacta |
| 92041 | aaagtacaaa aaaaattagc cgggcatggt ggcgggtgcc tgtagtccca gctacttggg |
| 92101 | aggctgaggc aggagaatgg catgaactcg ggaagcggag cttgcagtta gctgagatca |
| 92161 | cgccattgca ctccagcatg ggcaacagag caagactctg tctcaaaaaa aaaaaaaaaa |
| 92221 | aaaaagttta aaaactagac gttgacatga ttttacaata aggctgactg cttttgctac |
| 92281 | tttgccaatc agtccttagt gctttgttcc cataactgtg gtaagcaaga gcttacaaag |
| 92341 | aatacttaaa acaaacaaac aaacaaacaa aaaaaacact ttttctctct taatcagtcc |
| 92401 | agagaacctt taaaagaaac aagatcggcc agttgctgtg gctcatgcct gtaatcccag |
| 92461 | cactttggga ggctgaggtg ggtggatcac ttgaggtcag gagttcaaga ctggcctgac |
| 92521 | caacatgatg aaaccccatc tctactaaaa atacaaaatt agctgagtgt ggtggctatt |
| 92581 | tgagaggctg aggcaggaga atcatttgaa cccaggaggt gaaggttgca gtgagccaag |
| 92641 | atcacaccat tgcactccag tctgggtgac aagagcgaaa ctctatctca aaaaaaagaa |
| 92701 | aaaagaaaca agatcttcaa gcttaaggaa acaaaaacaa aactcagctg tgttaaatct |
| 92761 | gtttttagtt gctatacatt tctgctcagc ttcatgtgat gcacattcat gtaattgtat |
| 92821 | cctaaattcc tttgtacttt ttattttctt ccttggtctt caattatctt aagactacca |
| 92881 | agaaaacaaa aattttaaaa atcttcttca gccggtcagg cgcagtggct cacggctgta |
| 92941 | atcccagcac ttggggaggc tgaggcgggt ggatcacgag gtcaggagct caacaccagc |
| 93001 | ctggccaaca tggtgaaacg tcgtctctac taaaaataca aaaattagct gggcattgtg |
| 93061 | gcgcgttctt gtaatcccag ctgctcagga ggctgaggca ggagaattgc ttgaaccagg |
| 93121 | acccgggagg tgtaggttgc ggtgagcgga gatcgcgcca ctgcactcca gcctgggcta |
| 93181 | tagagtgaga ctccatttca aaaaaaaaaa aaaaaatctg cttcagctat tctgttaatc |
| 93241 | ttttgacatt acttagatgg tctggaaata aattttgaga ataacatgat tagaagtgag |
| 93301 | agagtataag catagttttg gagatacact cagaatagca ttatagattt tctcttttta |
| 93361 | ctaattggaa aaatggcagt tgttgaataa tagttttctt ccgtgaccct tgtgacttaa |
| 93421 | aaaaaaaaaa acactgaaat gaaataatcg aaccattttc tctaaacctt tgaatctgag |
| 93481 | ctctgcagtt aggtttataa tggtatatga aacctattag atatatactt ggaagtcata |
| 93541 | tgggatacaa accctgcttt tattatcttc cccttttgac taacttgggt ctcaagtttc |
| 93601 | cttaattact gcacagtgga ccttgatgtt gctataaaga atgtgtaggg ctgggcatgg |
| 93661 | tggctcatgc ctgtaatccc agcactttgg gaggccaagg taggcagatc acctgaggtc |
| 93721 | aggagtttga gaccagcctg gccagcatgg tgaaaccccg tctctactaa aaatacaaaa |
| 93781 | aaattagctg gttgtggtgg cgagtgcctt taatcccagc tactccagag gctgaggcag |
| 93841 | gagaatcact tgatacattt agttaggaga gaaaatcata cttatgttag taattgctgc |
| 93901 | tgttcttcat atacttgtgg ttttgattgc cagcaaattc ctaacatttt ggaaaagaaa |
| 93961 | acagtaatgg gataaagggt aagggctaga gaggacagtt ttatttacct agatcttcag |
| 94021 | agaagcctga agcctctttt aggaagtaac atttgaactg agaatgtaat aaatacattt |
| 94081 | tccctttctt ctagttccaa gattatcccc taaaactcat agacccaggt ctcccagaca |
| 94141 | gaacagtatt ggaaataccc ccagtgggcc agttcttgct tctccccaag ctggtattat |
| 94201 | tccaactgaa gctgttgcca tgcctattcc agctgcatct cctacgcctg ctagtcctgc |
| 94261 | atcgaacaga gctgttaccc cttctagtga gggtatgtaa caaagggctt ctggatccat |
| 94321 | aatctcagct gtgaaattga atgttagagg gtgatattat atgaaaaaat tctaggttat |
| 94381 | ttttattcat agacaagtat ttttagtgca catttaaaag tttatgtaaa ttttgatgtt |
| 94441 | gtttaatact actaatttaa tatagtgtct gtgttacaaa ggttaacatt cctgggtgtc |
| 94501 | aaatacctac ataaataaaa ttattggtgt ttcatatgac atctgcaaag gaaaaaaagc |
| 94561 | ctctgtttaa atgaaagcat tattttccaa aaacatagga aatcaaaatt attgttcagt |
| 94621 | gttttcttgt tttgcttttc taacttatct gaattttttt taaaaaattg ttttctagct |
| 94681 | aaagattcca ggcttcaaga tcagaggcag aactctcctg cagggaataa agaaaatatt |
| 94741 | aaacccaatg aaacatcacc tagcttctca aaagctgaaa acaaaggtta gagtttaaag |
| 94801 | agtcattaag cttaactgta ggaataggaa gaagtatgtc taatttcatg cccatacaga |
| 94861 | atatttttgt tcaacatttc ttcttactat tgtgatagat aaatgtattg cttgacaaat |
| 94921 | tccaaaatcc aaatttaata tttgaaatta ttttctgatc ttatatctta ttctaatttc |
| 94981 | tatcatctca tactaaaaag aatgtgatgt taaagtttaa aaataaacct gtgtcttaac |
| 95041 | agttcttaat tttacaggta tatcaccagt tgcttctgaa catagaaaac agattgatga |
| 95101 | tttaaagaaa tttaagaatg attttagggt aagtattgta ctaactgatg aatttgagtt |
| 95161 | ttagaaaata agcattacta aagatttatc tatttataaa aatgcgttat gtatacagtc |
| 95221 | agaaacatca aaccatatat gtagaaagca gaacattttt aaagtggtct ttgcctatcc |
| 95281 | tttaagtggg ataactaaaa tcatgagatt tggtaacaac aatatgtagg tatcaaatga |
| 95341 | gagtatagcc ctgacatttg aaaccaccat agcacagctt actatttgat ggtcatttgt |
| 95401 | actttgttca gtgaagctag atattagtag agcaaggcca agtcattaat aatctagtgt |
| 95461 | ggcaaatgga agatgtactg gactctggtg ttctgaggta gttggagatt tatactttgt |
| 95521 | acacaaatat attgtggtca aaatctttct gtaacattat ttctctgtct tagcacaggc |
| 95581 | tttacttaac atctctcctt gattgtcatt tcattctttt gcatgttact tactataggt |
| 95641 | atcgaggtag attttgagac caaccaataa atcttcttga aacttagctt cttagaaagg |
| 95701 | aaaatctaaa taccagcctt ttaaaaaaag tagctgaatt aaaggatgag tgaaccaaag |
| 95761 | gcaaaggtag cctttcctca gcctgtgttt tagctttcta aatgttaaca atagcttcat |
| 95821 | tcttgactta ttggtaacat tcaaaatact acttattatt tcatacttta gcacatgtat |
| 95881 | ctattcagct ttaatgctat taacagttgt taacctaagt tttcatttgt tggcgggcac |
| 95941 | ggtggctcac acctgtaatc ctagcacttt gggaggccga ggtgggcaga tcacctaagg |
| 96001 | tcaggagttc gagaccagcc tggtcaacat ggtgaaaccc tgtcttgacc aaaaatagaa |
| 96061 | aaattagcta ggcatggtgg cgcacacttg taatcccagc tacttggcag gctgaggcag |
| 96121 | gataatcgct tgaacccagg agacagaggt tgcagtgagc cgagatcaca ccactccact |
| 96181 | ccatcctggg cgacagagca agactgcatc tcaaaaaaaa aaaaaaaaaa aaaaagtttt |
| 96241 | tcaatttgtt aaacaatagt taacacatac aaatgataca aagaatattg aatatgatca |
| 96301 | tgtgcccact acccagctta gtaaataaag cattctaaca cagttaaact cctcttatgt |
| 96361 | atctgcccct cctcagctgc ttccccctgt ttccttccaa aaggaagggt ttcttttctg |
| 96421 | tgcagttctt tatatttata ctgcatatga atatatctgt gagcaataga tgatattttg |
| 96481 | cataatctta aatttgctat aaagtctttt tttttttttt aattgatcat tcttgggtgt |
| 96541 | ttctcgcaga gggggatttg gcagggtcat aggacaatag tggagggaag gtcagcagat |
| 96601 | aaaaagtgaa caaaggtctc tggttttcct aggcagagga ccctgcggcc ttccgcagtg |
| 96661 | tttgtgtccc tgggtacttg agattaggga gtggtgatga ctcttaacga gcatgctgcc |
| 96721 | ttcaagcatc tgtttaacaa agcacatctt gcaccgccct taatccattt aaccctgagt |
| 96781 | gacacagcac atgtttcaga gagcacaggg ttgggggtaa ggtcatagat caacaggatc |
| 96841 | ccaaggcaga agaatctttc ttagtacaga acaaaatgaa aagtctacca tgtctacttc |
| 96901 | tttctccaca gacgcagcaa ccatccgatt tctcaatctt ttccccacct ttcccccttt |
| 96961 | tctattccac aaagccgcca ttgtcatcat ggcccgttct caataagctg ttgggtacac |
| 97021 | ctcccagacg gggtggtggc cgggcagagg ggctcctcac ttcccagaag gggcggccgg |
| 97081 | gcagaggtgc cccccacctc ccggacgggg cggctggctg ggcgggggct gaccccccac |
| 97141 | ctccctcccg gatggggcgg ctggccgggc gggggctgac ccccacctcc ctcccggacg |
| 97201 | ggttggctgc cgggtggaga tgctcctcac ttcccagacg gggtggctgc caggcggagg |
| 97261 | ggcttctcac ttctcagacg gggcggctgc cgggcagagg ggctcctcac ttctcagacg |
| 97321 | gggcggccag gcagagacgc tcctcacctc ccagacgggg tcgcggccgg gcagaggcgc |
| 97381 | tcctcacatc ccagacgggg cagcggggca gaggcgctcc ccacatctca gacgacgggt |
| 97441 | ggccgggcag agacgctcct cacttcctag acgggatggc ggccgggaag aggtgctcct |
| 97501 | cacttcccag actgggcagc cgggcagagg ggctcctcac atcccagacg atgggtggcc |
| 97561 | aggcagagac gctcctcact tcccagacgg ggtggcggcc gggcagaggc tgcaatctcg |
| 97621 | gcactttggg aggccaaggc aggtggctgg gaggtggagg ttgtagcgag ccgagatcac |
| 97681 | gccactgcac tccagcctgg gcaccattga gcactgagtg aacgagactc cgtctgcaat |
| 97741 | cccggcacct cgggaggccg aggctggcag atcactcgcg gttaggagct ggagaccagc |
| 97801 | ccggccaaca cagcgaaacc ccgtctccac caaaaaaata cgaaaaccag tcaggcgtgg |
| 97861 | cggcgcgggc ctgcaatcac aggcactagg caggctgagg caggagaatc aggcagggag |
| 97921 | gttgcagtga gccgagatgg cagcagtaca gtctagcttc ggctcggcat cagagggaga |
| 97981 | ccgtggaaag agagggagag ggagaccgtg gggagaagga gaaggagggg gagggggagg |
| 98041 | gggggagagg gagagggaca atgatgtctt gctgtaggta ttcttcccca tttgaatttt |
| 98101 | ttcctcagca ttattttttt taacatcatt cagtctcctc ttatactaca cttggattga |
| 98161 | atttaatatc tcatgaagaa aaaacatttc tactttgaag catgtgaatt agcatgtttt |
| 98221 | tataacagct ttattgagat ataatttaca tacataaata aaccgtttaa agtgtataaa |
| 98281 | tcagtggttt ttaatgagat ataatttaca tatataaatc aaccatttaa agtgtataaa |
| 98341 | tcagtggttt ttaaaatatt cacaatgttg tacaaccgtc ttctcagttg attttaaaac |
| 98401 | atactcttca cccccaaaag aaaccccgtg cccagtttag cagtcgttcc acatttgcct |
| 98461 | ccagcccttc tctttcccct actcccaacc ctaagcaacc gttaatctac tttctgtctc |
| 98521 | tatggatggg cttatttggg gcaaattcca tttcatacaa atggaataat aaaatatgtg |
| 98581 | gcttttatga ctggcttctt tcactcagag tagtgttata aaagttcatc catgttggag |
| 98641 | catgtttcag tacttcattt ctttttgtga ctgactaata ttccttgatg tggataatac |
| 98701 | cacattttgt ttatccatta atcagtttgt agctatttgt ggtgttctca ctgtttgact |
| 98761 | attctgaata acactgccac aaacatgagt gtgcagtttt tttctcgtcc tatcttttca |
| 98821 | tttcttttgt gtacctacct aggagttgaa ttgctgggtc atatggcaac tgtgtttaac |
| 98881 | cttttgagga actaccaagc tatttgccaa gatatctaca ctattttaca ttcccaccag |
| 98941 | cagggtatga gggtttctgt ttctccacat ccttgctaac acttattgtc ttgtcttttt |
| 99001 | tgattatagt catccttgtg ggtgtgaagt gttaacctca ttgtggctct aatgtgcagt |
| 99061 | tctttcatgg ctaatgatgt tgaacatctt ttgtgtttat tggccattta tatatcttct |
| 99121 | ttggattgat gtctgttcaa atctttaccc attttaaaaa ttgagttgtc tttttattat |
| 99181 | tgggttgtgg gagttcttta tatattgtgt gtacaagtcc ctgttagata catggtttgc |
| 99241 | aaatgttttc tcctgttctg ttggttgtct ttttactttt tcatcccttg aagcacaaaa |
| 99301 | atttttaatt ttgatgaagt ccaatttatc tgattttgaa gtaagctttt ggtgtcgtat |
| 99361 | ctaagaaaat actgtttcat caatcattaa ggtttattac tcttctgggt ttttttaaga |
| 99421 | attacattta gaggtgtgat ccatttggag caactttttt tttcttttga cacagaatct |
| 99481 | cgctcttttg cttaggctgg agggcagtgg tgcaatcttg gctcacagca gcctcagcct |
| 99541 | cctgggctca aatgagtagc tggtactaca ggtgtgcacc accacacctt gctattaata |
| 99601 | acttttgtat ttttttgtag agacagaatt tcgccatgtt gcccaggctg gtctcaaaca |
| 99661 | cttggactca agtgacacgc ccacctcagc ctcccaaagt gaaaaattgc tttcaccttg |
| 99721 | cactgcggac tcgccctgaa ttctttcttg tgcaagatcc aagagccctc tctgggggtc |
| 99781 | tggatcggga cccctttcct ataacaatat tatgagaata acatttgatt ttttttaagt |
| 99841 | gaaacaaatt gttattaaaa aattaaaaaa ggtcatagga gagtgacttg gtgctcagcc |
| 99901 | cattttgagc agttatttaa tatagcataa ggtggggttc aaattcattc tttatattaa |
| 99961 | ttttttattt ctaattgaca cataaccata cacttataac catttttact gtgtaagttc |
| 100021 | agattcattc ttccgtatgt aggtattagt tgtcccagca ccatctgtta aaaagactat |
| 100081 | tcttggccag gcacagtggc tctcaacgcc tgtaatccca gcactttggg agtcccaagc |
| 100141 | aggcagatca catgaggtca ggagttcgaa accagtctga ccaaatggtg aaaccgcatg |
| 100201 | tctactaaaa atacaaaaat tacctgggtg tggtggcgca cacctgtagt ctagtcccac |
| 100261 | tactgtagtg gctgaggcag gagattcgct tgaacccagg aggtagaggt tgcagtgagc |
| 100321 | tgagatcatg cactccagtg tgggcgacag agtgagactc catctcaaaa aaaagactat |
| 100381 | tctttcctcc attgaattat cttcacatgc ttgttggaag tctgttgact acaaatgtga |
| 100441 | aagtttatta ctggactctg aattgtcctc cactgaatct ctatgtctta tccttatggc |
| 100501 | agtaccatac tgtcttgatt agagttactg tattttaaaa ggctgtactt tttcagttag |
| 100561 | cagaaaacat tttagctatc agcacaactt tctgtaaacc ttcattaatg cttgacttaa |
| 100621 | attccaagaa ggagcaacat aaaaagtctt atctctttag gagttttagt cttactactt |
| 100681 | ttaggtgcct gaataaccaa atgtattatt tagcctctta ctaataactc cttgatccat |
| 100741 | aggggcatac caggaagaaa agaagtggtt tttaaaaaat gagagtgggc cgggcacggt |
| 100801 | ggctgatacc tataatccta acactttggg aggctgaggc gggtggatca cttgaggtca |
| 100861 | ggagtttgag accagcctgg ataacatggc gaaaccctat ctttattaaa aatatataaa |
| 100921 | ttagccgggc atggtggcac atgcctgtaa tcccagctac tcaggaggct gaggcaggag |
| 100981 | aatcacttga atccaggagg tggaggttgc agtgatccga gattgcatca gtgggcgaca |
| 101041 | gagcgagaat ctgtctcaaa gaaaaaaaaa gagagtggaa aaaaaaaata tgtgtcccag |
| 101101 | aacttaaatt ttaattaaaa aaaaataaaa gagtgaactt tctaattgtt ctcttcagat |
| 101161 | aatataatgt tattctctta tgttttattg cgtatttcct gtgtaccaga tgctgttctt |
| 101221 | catgcttgta tgttaaatct tgtctaacat ctctgtcaag caagttctgt ttgtatctgc |
| 101281 | actgtgtata ttaggcagct tgggcaaaga gaagttaagt aatctgccca aactcacatg |
| 101341 | gctagtaagt aagagggctg accatctggt gtttaagctt ctagcagtgc tttgaatagt |
| 101401 | aactaatgca tagtgcatgc tgcactgtca gtcagtgatt cattagagct aacttcatga |
| 101461 | catgctcata gccccaaact gcatttgttc acaaatatct gtagtccttc atttaggcag |
| 101521 | aaatagaaat accttgtgtg tttgttgttc cttccctttt gagccatatg cagagtgctg |
| 101581 | atagctttat ttgtgtaaga attgctagta atttgatctg ttttgggtta ataatgtggg |
| 101641 | ttttagaggt aaatggacct aggtttgaat gttggcctct atacatcatg tgcgtaacat |
| 101701 | tgtggcatgc tatctacttc ccccaagcca aaatgggtta attttagaac ctgcttcata |
| 101761 | gtgttcctgt gagagctcga tgagatattg cctataaagt gtttagcata gtgcctagca |
| 101821 | catggtatgt attcaataca tgttcattct tactagcaaa atatagatga cccagtattg |
| 101881 | tacagagtat gtacaatggt gtcattgtac catttcatgt ggagtcacat aagaatttca |
| 101941 | gttttctgct gggcatgatg gctcactcct gtaatcccag cactttggga ggctgaggtg |
| 102001 | gatggatcag ctgaggtcag gagttccaga ccagcctggc cgacatgatg aaaccccatc |
| 102061 | tctactaaaa atacaaaaaa ttagccaggc gtggtggcag gtgcctgtaa tcccagctac |
| 102121 | tcgcaagact gaggcaggag aaatgcttga acccgggagg cggtggttgc catgagttaa |
| 102181 | gatcgtgccg ctgcactcca gcctgggcaa taagagcgaa actccgtctc caaaaaaaag |
| 102241 | aaaaaaaaag aacttaagtt ttccattaga tttagtatag tgcagagagg aaatacagca |
| 102301 | gagtgctata ttccatatat agcaatatag cattagaaca atatattcca atacagcaga |
| 102361 | gtgctatatt cagataccaa ctagtggact tgctatttgt aagatggcaa taatagtatc |
| 102421 | tacatcaaat agggctgttg tgaagactaa atgaataagt ctataaatag tttagaacag |
| 102481 | tgtctggaca ggtacagtgg ctcatgcctg aatcttagca ctttgggagg ctgagacagg |
| 102541 | tggatagctt gagctcaggc attaaagacc aacctgggta acatggtaaa accctgtttc |
| 102601 | tacaaaaaaa tacacacatt agccaggtgt ggtggcacat gctaatagta ccagctactc |
| 102661 | aggaggctga ggtgggagaa tcacttgagc ctgggagatg gaggttgcag tgaggtgagc |
| 102721 | ttgcaccact gcgctccagt ctgggcaacg gagtcagacc ctgtttggaa aaaaaaaaaa |
| 102781 | aagtgtccaa cccatagtaa gaaatgcaga tgtgtttgac attgtaagaa aaagcaacac |
| 102841 | caaaagtctg atttttgcct tcactcaaga actcttatga taattaaact ccgaagtcct |
| 102901 | tggcaatata tatagttggt ctgttatgtg gatcgcctct actaaagatt tttgtgaaca |
| 102961 | aatgaaagtt taagtagtaa gttcctacat cgtgacttaa attgccagtg tgcccacata |
| 103021 | aataccctgt caacatttgc ccttagccac ttgactcttt agctatattg gtaatgcagt |
| 103081 | aaagcttgcg atgcgccaga gttgcataat gctgtttgcc atgacaccaa gagccttggt |
| 103141 | aatgaaacca ttgaaattgg tttgcctata ctgaggctga agaggtatct tggctctcta |
| 103201 | attttaaggc aacctttttg gctgtgtagg tttctcttta gcttgtttct caccacctgg |
| 103261 | ggctgtggct taggtccgtt gtcctaacct gtggcttagg ttctgttttt gttgcttgta |
| 103321 | cttgctcccc cttttttcag ccattcctgt tttctttctt ttgtagagga tgccatctta |
| 103381 | aatcatcttc agccagtggt agcattttat tttttctggt ctgcaaactt aaaaacctca |
| 103441 | tcacttattt tgctaatatc tttgtcttct gttctttttg atggtccttg gttttgcagt |
| 103501 | ctactttaaa ggtttttatt tttttatggg tacatagtag acgtattact catagggtct |
| 103561 | gtgagatatt tagataaagg catataatgt gtaataatca cattagggta aatggggtat |
| 103621 | ccatcaccat catcattcat catttctttg tgtaatgaac gttgcaattg tactccctca |
| 103681 | gttattctaa aaagtacaac aaattaatgc tgactgtagt caccctgctt tgttgtcaaa |
| 103741 | tactagatct tattcattct ttatttaact ttttaaattt taaacttatt ttatttattt |
| 103801 | atttttagac ggagtctcac tctgtcgcca ggctggagtg cggtggcgca gtctcaactc |
| 103861 | actgcaacct ccgcctccag ggttcaagtg attctcctgc ctcagcctcc tgactagctg |
| 103921 | gaactacagg cacgtgccac cacgcccagc taatttttgt atttttagta gagacggggt |
| 103981 | ttcactatgt tggctgggat ggtcttgatc tcttgacctt gtgatccggc tgccacagcc |
| 104041 | tcccaaagtg ctggggttgc aggcgtgagc caccgtgccc ggcctttaaa attattttaa |
| 104101 | atcattttaa tatctttttc atttctgcct ccggtcctgc agagttctta ttcgttcttt |
| 104161 | ctaaattttc tttgcaccca ctaatcacct catttccctt cttctcccca ttacccttcc |
| 104221 | caacttctgg taaccattct gctatctcca tgtgttcaat tgtttttatt tttagtgcct |
| 104281 | gcaaacgagt aagaatatgc aaagtttatc tttctgtccc tggcttattt tacttaacat |
| 104341 | aatgtcctcc agtgccatct acattgctgc aaatgacagg atctcattct tttttatggc |
| 104401 | tgaatggtaa tctattgtgt atatatacca cattttcttt ctccatttgt ctgtcagtgg |
| 104461 | acacgtaggt tgattccaaa tcttggctgt tgtgtatata gtgccgtagt aaacatggga |
| 104521 | gtgcagatat tccttcaata aactgatttc ctttctgagt atatacctag cagtgcaatt |
| 104581 | gctggatcat atggtagctc tatttttagt tttttgagga atttccatac tgttctccat |
| 104641 | agtggtttta ccaatttaca tgtccaccaa cagtgtgtga aggttcccct ttatccacat |
| 104701 | cgttaccagc atttgttatt gcctgtcttt tggataaaag ccattttaac tggggtgaga |
| 104761 | tgatatcttg ttgtagtttt aatttccatt ttcctggtga tcagtagtat tgaatacctt |
| 104821 | tcatatacct gtttgccatt cataaataac gatgaggtct tgctgtttgg cccaggctgg |
| 104881 | tctcgaactc ctgggctcaa gcaatcctcc caccttggct tcccaaaatg ctgaaattat |
| 104941 | agttgtgagc cactgcacct ggccttgtat gtcttccttt tttttttgtt ttgttttgtt |
| 105001 | tttgagacag agtctcactt tgttgcccag gctggagcgt agtggtgtga tcttggctca |
| 105061 | ctgcgcccta cacctcccgg attcaagcaa ttctcctgcc tcctgccacc atgtctgcct |
| 105121 | aatttttgta tttttagtag agacgggatt tctccttgtt gcccaggctg gtcttgaact |
| 105181 | cctaacctca ggtgatttac ctgcctcagc ctcccaaagt gctaggatta caggcgtgag |
| 105241 | ctgctgcgcc cagcctgtat gtcgtctttt gagaaatgtc tattcagatc ttttgcccat |
| 105301 | ttttaattga gttactaaaa ttttccctat ggagttgctt gagtgccttt tatattctgg |
| 105361 | ttattgatcc cttgtcagat gagtagtttg caaatatttt ctcccattct gtgggctgtc |
| 105421 | tcttcacttt gttgatggtt tcctttgctg tgcagaagct ttttaacttg atgtgatccc |
| 105481 | atttgtccat ctttgctttg gttgcctgta cttttggggt attactcaag aaatctttgc |
| 105541 | ccagagtaat gtccctggga gtttaatgtt ttcttttagt agtttcatag tttgaggtct |
| 105601 | tagatttaaa tctttagtcc attttgattt gatttttttt taatatggtg ggacacaggg |
| 105661 | gtctggtttc attcttctgc atatggatat ccagttttcc cagcaccatt tattgaagag |
| 105721 | actgtccttt ccccagtgta tgttcatggc ttctttgtgg aaaatgagtt cacttagacg |
| 105781 | tatggattca tttctgagtt ctctgttctg tttcattgat ctatatcttt ttttatgcca |
| 105841 | gtaccatgcc attttggtta caataatttg aagtcagata atgattcccc ccgttttgtt |
| 105901 | cattttgctc agtatggctt ttgctctttt gggccttttg tggttcccta caaattttag |
| 105961 | aattattttt gtctacttct gtgaggaatg tcattggtat tttgataggg attgcactga |
| 106021 | atctgtagat tgctttgagt attatcaaca ttctagcaat attaattctt ctaatccata |
| 106081 | aacatggaat ctcttttcat gttttttctg tgtcatcaat ttcagtgttt taaagttgtc |
| 106141 | attatagaaa tcttttactc atttggttaa gtttattcct aagtatttta ttatatttgt |
| 106201 | agctattgta aatgggattg cgtttaaaaa atttttcaga ttgtttgctg ttaaatataa |
| 106261 | aaatgctcct gatttttgtg tgttgatttt tgtatcctgc aattttactg aatttgtttg |
| 106321 | tcagttctaa taggtttttc ttttttggag tctaggtttt tccaaatgta agatcatatt |
| 106381 | atctgcaaac aaggataatt tgacttcttc cattccagtg tggatgcttt ttatttcttt |
| 106441 | ctgttgtctg attgctccaa ttaggacttc cgagtattat gttgaataac aatggtgaaa |
| 106501 | gtgggcatcc ttgtcttgtt ccagatctta gaggaaagcc tttcagtttt tcccttttca |
| 106561 | gtatggtact agttatgggt ctgtcatata tggcttctgt tttgttgagg tatattcctt |
| 106621 | ctatacccag ttctttgggg tttttttgtt tgtttgtttt tgagatggag tctcactctg |
| 106681 | tcacccaggc tggagtgcag tggcgcaatg ttggctcact gcaagctcca cctcctgggt |
| 106741 | tcatgccgtt ctcctgcctc agcctcccga gtagctggga ctacaggtgt ccgctaacac |
| 106801 | gcccggctaa ttttttgtat ttttagtaga gacggggttt caccgtgtta gccaggatgg |
| 106861 | tctcgaactc ctgacctcat gatctgcccg tctcagcctc ccaaagtgct gggattacag |
| 106921 | gcgtgagcca ccacgcccgg ccaagggttt taatcataag gggatgtggc attttatgtg |
| 106981 | atataaatta tatatttata tcatgtgata tatatttata tcatacacag tataaataat |
| 107041 | atatatatat attttttagt ctttgtcttt tactctgtta agatgtacca tgtttattga |
| 107101 | tttgcgtatg tcgaaccatc cttgcatccc tgggatgaat cccacttagt catgatgaat |
| 107161 | gatcttttta atgtgttact gaattcggtt tgctagtatt atattgagga tttttgcata |
| 107221 | atgttcttca gagacactgg cttctagttt tccctttttg atgtgtcctt tggttttgta |
| 107281 | tagggtaata gtggccttgt agaatgagtt tagaagtatt ccctcttcct gtattgtgtt |
| 107341 | ggaatagttt gagtaggatt ggtattagtt cttctttaaa ggtttagtag aattcagcag |
| 107401 | tgaagccatc aggtccatgg cttttctttg ctgggagact atttcttata gctttgatct |
| 107461 | cgttacttgt tattggtctc gttacttgtt attgtatttg ggttttggat ttctttgtgg |
| 107521 | ttcagtcttg gtaggttgta tgtgtctagg aatttatcca tttcttcaag gttttccaat |
| 107581 | gtatcagcat atagatgctc atagtagtct ctaatgatcc tttgaatttc ggtggtaaca |
| 107641 | attataatgt ctcctttttc atctctcatt ttattatttg ggttttctct tttttttctg |
| 107701 | agtctggcta aaggtttgtc agttttgttt atctcttcaa aacaatttac tgttttattg |
| 107761 | atcttttgta ttttcttcat ttcaatttta tttatttctg ctttgatttt ttttatttct |
| 107821 | tctactgatt ttaggttttg tccttgcttt tctagttctt taggatgtat tggcagatga |
| 107881 | agtttttcca cttttttgat gtaggcactt actgctgtaa acattcctct tattgttgct |
| 107941 | tttactgtat cctataggtt ttgataagct gtgtttccat tttcatttgt ttcaaggaat |
| 108001 | tttccagttt tcttcttaat ttcttcatgg acccactggt cattcaggag catattgctt |
| 108061 | aattttcatg tatttgtata ctttccaaag ttcctcttgt tatctagtgt tattttattt |
| 108121 | tatttttatt tttgtttttt tgagatggag tctcgctctg tcacccatgc tggagtgtag |
| 108181 | tggcgcgatc tcggcttact gcaacctctg cctccccagt tcaagtgatt cttctgcctc |
| 108241 | agcctcctga gtagctggga ttacaggcat gtaccaccac tcctggctaa tttttttttg |
| 108301 | tatttttagt agagaggggg tttcaccatg ttggtcaagc tgatctcgaa ctcctgacct |
| 108361 | cagatgatcc acccaccttg gcctcctaaa gtgctggaat tacaggcatg agccaccgtg |
| 108421 | cccggcctct agtgttatct tattgtgatc agagaagata gttgatatga ttttaacttt |
| 108481 | tttgaatttt tatttattta tttgtttgtt tgtttgtttg tttgtaacag agtctcactc |
| 108541 | tgttacccag gctggagtac atgtcatgat cttggctcac ctgcaacctc cgccttcctg |
| 108601 | gctcaagcaa tcctcccacc ttagccttcc aagtagctgg gactacaggc acatgccgtc |
| 108661 | acatatggct gatatttttg gatttttttt ttttttgtag agatggggct ttgcgatgtg |
| 108721 | tcccagggtt gtttcgaact cctgagctca agcaatccac ctatttcggc ctcccaaggt |
| 108781 | gctgggatta cagacatgag ccactgtgcc acgtcaaatc tttagactcg ttttgtggct |
| 108841 | taacataggg tctatctttg agagcaatcc atatgttgag gagaagaatg tgtattctat |
| 108901 | agctgttgga cacaatgttc tgtaaatatg tattgggcct atttggtcta tagagcaaat |
| 108961 | taggtctaat gtttctttgt tgattttctg tctgaatgat ctgtccattg ctgagagtgg |
| 109021 | ggtgttgaag tttccgactg ttactgaggt ctgtttctct tttttgctct aataatgttt |
| 109081 | gctttatata tctggatgct ccagtattgg ttgcatatgt atttatactt gttataacct |
| 109141 | cttgccgaat tgatcccttt atcattatac aataatcttc tttgtctgtt tttatagact |
| 109201 | ttgtctcaaa atctatttta tctaagcata gctactcctg ttcttttctg gtttccattt |
| 109261 | gcatggaata ttgttttcca gctcttcaat tttagtctat gtgtgatttt ataggtaaag |
| 109321 | tgtgtttctt gtaggcaatg gatctttggt tttttttttt ttttttttga gacagagttt |
| 109381 | tgctattgtt gcccaggctg gagggcaatg gcgctatctc agctcactgc aacctccgcc |
| 109441 | tcctgagttc aagcgattct cctgcctcag ccccccaagt agctgggatt acaggcgcct |
| 109501 | gccaccaagc ccagctaaat tttttgtatt ttcagtagag atggggtttc agtatgttcg |
| 109561 | tcaggctgtt cttgaactcc taacctcagg tgatttgcct gccttggcct cccaaagtcc |
| 109621 | tgggattaca ggcgtgagcc accgcaccca gccttttttt taaatccact tagccactct |
| 109681 | gtatcttttg attggagagt ttagtcgatt tacattcagt gttgttactg attagtgagg |
| 109741 | acttaactac taccattttg ttacttatta tctggttgtt ttgtagtcct actccctccc |
| 109801 | ttcccccttc ttttttactt cctcttcgct ccttttttcc ctccctccct tccttgtttt |
| 109861 | gaaagtgatt ttctctggtg gtatgtttta atttcctgct ttatattttt tgtgtatctg |
| 109921 | ttgtaggtgt ttttgattta agatcaccat gacagctggg tgcagtggct cacacctgta |
| 109981 | atcccagcac tttgggaggc cgaggtgggt ggatcaagag gtcaggagat tgagaccagc |
| 110041 | ctggctaaca tggtgaaacc ccatctctac taaaaataca aaacttagcc aggcgtggag |
| 110101 | gcacgtgcct gtaatctcag atactcagga ggctgaggca ggagaattgc ttgaacccag |
| 110161 | gaggcagagg ttgcagtgag tcaatattgt gccactgcac cccagcctgg gcgacagagt |
| 110221 | gagactccgt ctcaaaaaaa aaaaaaaaaa agagatcaca taagggttgc aaataacatt |
| 110281 | ttataaccca ttattttaaa ccaatgacaa cttgaaactt tgattgcaaa aacaagcaag |
| 110341 | caaagagaaa actaataaaa actctacact tcatctgccc gctttttaac ttttgttgtt |
| 110401 | tttatttata tctttattat actatgtctt aaaaaactgt agttataagc caggcgcagt |
| 110461 | ggttcacgtg tgtaatccca gcactttggg aggctgaggt gggcggatca cctaaggtca |
| 110521 | ggagttcgag accagcctag ccaatatggc aaaaccccct ctctactaaa aatagaaaaa |
| 110581 | ttagccggac atggtggcgg gtgcctgtaa tcccagctac tcggaggctg aggcaggaga |
| 110641 | atcacttgaa cccaggaggc ccaggttgca gtgagccgag agtgcgccac tgcactccag |
| 110701 | tctgggcaac agagtaagac tgtctcaaaa aacaatacaa aacaaaacaa aaccctggcc |
| 110761 | tagtggctca cgcctaatcc cagcactttg gaaggcaaag gtggggcgaa tcacaaggtt |
| 110821 | aggagttcga gaccagcctg accaacgtgg tgaaactctg tctctactaa aaatacaaaa |
| 110881 | attagccagg cgtggtggca cgcacctgta atcctagcta ctcaggaggc tgaggcagga |
| 110941 | gaatcgcttg aacctgggag gcggaggttg cagttagccg agatcgcgcc actgccgtcc |
| 111001 | agcctgggca gcagagcaag actctgtctc acaaaaaaaa aaaaaattgt agttcttatt |
| 111061 | tttgaaaggt tcatttttta ttcttcctgc tcaaaatatg agtagtagct tatacaccac |
| 111121 | aattacagtg ttacaatatt ctgtattttt ctgtgtactt gttaccagtg agtttttgca |
| 111181 | ccttcaggtg atttattatt gtttgttaac atccttttct tgcagattga agaacttttt |
| 111241 | tttttttttt ttttttttga gacagagtca tgctctgtta ccagcctgga gtgcagtggt |
| 111301 | gccatcttgg ctcactacaa cctccaactc ccaggttcaa gcgattcttc tgcctcagcc |
| 111361 | tcccaagtag ctgggattac aagcatgtgc caccacgccc agctacttct tgtattttta |
| 111421 | gtaaagacgg ggttttgcca tatttgccag gctggtcttg agctcctgac ctcagggtga |
| 111481 | tccgcccgcc ttggcatcct aaagtgctag gattataagc gtgagtcatc gtgcccaact |
| 111541 | tggttgttta ttttcaaata gcctgaattc aagctcacta atgttttctg ctgcttgata |
| 111601 | catttctgct attgagagac tgatgcattt ttcagtttgt caattgaatt tttccacttt |
| 111661 | gggatttctg cttgattctt tttactaata attattgcag tctctttttt aaatttatag |
| 111721 | gattctgaat ttgttctctg tattatcttg gatttcgttg aactttctca aagcattcag |
| 111781 | cttgaattct gtctgaaagt tcacatatct cttatcactt gggaattggt cactggtgtc |
| 111841 | ctttattttt agttcatttg gtgaggtcat gttttctcag atggccttga tgcttgtgga |
| 111901 | tgttcatcag tgtctgggca ttgaagagtt gggtattctg ttctttgtag tctggttttg |
| 111961 | tttgtacgca ttcttttttt tttttttctg tttttgagac agagtctcgc tctgtcgccc |
| 112021 | aggctggagt gcagtggcac agtctttgct caccgcaacc tccgtctccc ggattcaagc |
| 112081 | aattctcctg cctcagcctc ctgagtagct gggattacag gtgcgtgcca ccacgcctgg |
| 112141 | ctaatttttg tatttttagt aaatatggtg tttcaccatg ttggtcaggc tggtctcgaa |
| 112201 | ctcctaacct cgtgatctgt ccgccttggc ctctcagagt gttgggatta caggcgttag |
| 112261 | ccactgcatc cggctcccat tcttcttgag aaggtttttc aagtattcaa agggaattaa |
| 112321 | gtgttgtcat ctaagtcttc gctcactgca gccatacatg cattagaggg caccccaaga |
| 112381 | ctagtaatgt tgtgactctg tagaggtatc accttggtag tcttggggaa gatctgggag |
| 112441 | aattccctgt attaccaggc agtctcttgt cctcttacat ttctccaaac aaatggagtc |
| 112501 | tctctttgtg ctgagctgct tggagtttgg ggaagggtga cacaagcact gccatggcca |
| 112561 | ccgtcactgg aactgtactt ggtctcaccc aaggcctgtg gcagctattt tctggccacc |
| 112621 | actgatgtta atttaaggcc caagggtgct ttagtcagta ggtgaagaat cctgcaagaa |
| 112681 | ctgggtcttt actttcagtg cagcaggttc ccttctggcc cagggtgtgt ctagaaatgc |
| 112741 | tgcccaggag ccagggcctg ggatcgggag ctttaggaat ctgctttatt gtactggggc |
| 112801 | tgagctggca cccacttgca agataaagtc ctttttactc ttctctcacc tcaagcaggt |
| 112861 | gggtctcccc atggacacca cagctgtgaa tgtgcggggt catatctgaa gctggcacaa |
| 112921 | tacgacatgg caccttgttt tttattcaag gcacaagggc tctttagtca gctggtggtg |
| 112981 | aatcctacta ggactaggta tttcccttca aggcaatggg ttcccttctg gtccagaata |
| 113041 | tgtctagaaa tgtcatctgg gagctatggc ctagaattga ggcttcagaa ctatgcttgg |
| 113101 | tgctttattt tactgtggct gaactagtat ccacattgca agacaaagtc ctccctactc |
| 113161 | ttccctctcc tcccagagct gtgagctgtg gtacctggag ttgggggaag gctggcacaa |
| 113221 | gcactccctt ggccacccta gctggtgtct cagtgggtca catgtacccc aagtccactg |
| 113281 | actatgagcc cagcacagta ccatgacttg tccaggaatt gcagtccttc tggtctagac |
| 113341 | tgcctttcaa gtttatttag gaccccagag gactttaccc acggtggtgg ggcttaccaa |
| 113401 | aattaagatt cttttggttt tttttggcag agtttcgctc ttattgccca ggctggagta |
| 113461 | tagtgacgca atctcagctc accacaacct ccgcctcccg ggttcaaata attctcctac |
| 113521 | ctcagcctcc tgagtagctg ggattaccgg catgcgctac cacctctggc taattttttt |
| 113581 | gttttttagt agagatgagg tttctccatg ttggtcaggc tggtcttgaa ctcccgacct |
| 113641 | caggttatcc gtccgcctcg gcctcccaaa gtgctgggat tacagaccat agtgcccagc |
| 113701 | ccgaaattca gattctaatc actgggatgg acaattcccc tctgactagg gctagtctaa |
| 113761 | atactccctc tgtgggtgct ggctgaattc tgtcctatgc tgctttccac tgtgacaggg |
| 113821 | cagcactgag tttcaatgca aaatcccaca gtcatttctc tctctctccc ccgagcacac |
| 113881 | agattctttc tccaccccac actgcattgt gggggaatgt caggggtgct ggaggggcag |
| 113941 | ttcaagacta tcttccttat cttttttggt gtctttttcc ttgataggat gtcaaaactg |
| 114001 | ggtactgtga tcgcttacct aatttttggt tcttatgaag gtgctttctt gtgtggatag |
| 114061 | ttgttcaatt tggtgctcct tgttggggat gatcactgga aggttctgct tggccaccat |
| 114121 | gctctgtctc ttctcccctg ccatctcctt tttttactta ggggtttaga atgtctaact |
| 114181 | gaccaatatg tacagtcagg tctcattctg aattcaccta cttaatgacc ttccaagctg |
| 114241 | actaggccca gcgcttagtc cagcctccat gacggtccct ccacatccta attagcctcc |
| 114301 | ctccagttca tttcacacaa agctgctgtg ttcacctttc tgaactataa atctgcccag |
| 114361 | tactctaccc tacttaaaat tccgtataga ctgcccattt gccctgagaa ttaaaagcca |
| 114421 | aagtcctaaa cgtagctttt taaaactttt tttttttttt ttttaatttt tagatggagt |
| 114481 | cttgctctgt cacccaggct ggagtgcagt ggtgtgatct tggctcactg caacctccgc |
| 114541 | ctcctgggtt caagcaattc tcatatgtca gcctcccaag tagctgggat ttacacgtgt |
| 114601 | gccatcacgc ctggctaatt tttttttttt atctttagta gagacggagt ttcaccatgt |
| 114661 | tggccagtct ggtcttaaac tcctgacctc aagtgatcca cctgccttgg cttcccaaag |
| 114721 | tgctaggatg ataggtgtta gccactgcac gcagccctga acatagctct taagttcctt |
| 114781 | tattgtcata ttccttttga cgagtctatc attttctgac tcacttgtac atgtgtgtct |
| 114841 | cacccttggt ccagccattg gtgcttttct ttacttcttt atttttgtta ttttatttta |
| 114901 | ttttattatt attttttaaa tgagacaggg tatcactatg ttgcccaggc tggtcttgaa |
| 114961 | ctcctgagct taagcagtct gcttgtctca gcctcccaaa gggctggaat tacagtgatg |
| 115021 | agctactgtg cccagctcat tggtgctatc tttttttttt tttttgagac ggagtctcgc |
| 115081 | tctgtcaccc aggctggagt gcagtggcgt gatcttggct cactgcagct ccacttccca |
| 115141 | ggttcacacc attctcctac ctcagcctcc cgagtagcag ggactatagg cgcctgccac |
| 115201 | catgcctggc taatttttgt atttttagta gagatggggt ttcagcgtgt gagccaagat |
| 115261 | ggtctcgatc tcctgacctc gtgatccgcc tgccttggcc tcccaaagtg ctgggattac |
| 115321 | aggcgtgagc caccgtgccc ggcccccatt ggcgctattg ttttatgtga tagagccagc |
| 115381 | ttctcccttt tctttggatt tttaaacata ctcttccttt tacttagact attctccatc |
| 115441 | ccaacacctt tcctaaactt ctttcacacc ttagactagc tgacacttta ctgagaaacc |
| 115501 | tttctttttt ataggttgct ttttctatag actctcttag catttactca ttttattgtg |
| 115561 | aagtgtctga tcttatttaa atgacaagta taagaggata gaaactattt catatttttc |
| 115621 | tcacccagca ggcacaattt ctgacatgtg gtaagcactc agtaaatatt gaactttaga |
| 115681 | ggctaggaca tttgagtgct ttggtgactg tggttgtgct atataggtac tctgttattg |
| 115741 | ttagtttata gtaaaagcat tactcttaaa gtatgaaaaa agccttattc agaacatttc |
| 115801 | atgcgtatag ttaatattac gtagcttgtg ctcatggcaa aaatgtatta ctaaagttat |
| 115861 | ttaagatatt taagtataat tgtttccttt atttagttac agccaagttc tacttctgaa |
| 115921 | tctatggatc aactactaaa caaaaataga gagggagaaa aatcaagaga tttgatcaaa |
| 115981 | gacaaaattg aaccaagtgc taaggattct ttcattgaaa atagcagcag caactgtacc |
| 116041 | agtggcagca gcaagccgaa tagccccagc atttcccctt caatacttag taacacggag |
| 116101 | cacaagaggg gacctgaggt cacttcccaa ggggttcaga cttccagccc agcatgtaaa |
| 116161 | caagagaaag acgataagga agagaagaaa gacgcagctg agtgagtaaa cctggaactt |
| 116221 | agaccatcct gttactcaat taactttttt ttttttaaag gcatttaggt ccttccaact |
| 116281 | gtgaagaatc catctggact tttagactac tttatacatt gcccttagct tacaaacagc |
| 116341 | tagtccaaac aaatgacatc ttaagtaaat gaggttattg caccctgtgc tactcttctg |
| 116401 | ttcttcccct tttttgtacc ccagggctag aaaaacaagg cataaattaa gaaaagtttt |
| 116461 | tctgtaaatg aacaggagtt gaaaaattat caattcaggg gacctatctt tactggattc |
| 116521 | cactcattag tcaccctcac tgtgctgcta ggctgaaaaa ctgccactgt caaggagaga |
| 116581 | agcatgcggt gcttctactt ggaattcaaa atatttttca tcagaaactg tgttttagtt |
| 116641 | aatgtttaga tttgttaaga tagacttaat tctgcacatt cagtatatta attaaatgga |
| 116701 | cttttagggg ctaacctcag aacttaacta ccattgactt aggtgtttgg gtaccaaaca |
| 116761 | atccagttaa agctgaagtt ttggaatgca gcttattgat aaattgggga ctgcttattc |
| 116821 | ttgatttgag gcaatttttt tttacagcca tgactttttc caggtatgtc atgtaaaata |
| 116881 | tcttctcaca taagaattac tgcatgctag aatattggta tgttgactgg tagctcatac |
| 116941 | ctataatccc agcactctgg gaggtccaag caggtagatt acttgaggct aggagttgaa |
| 117001 | gaccagcctg gccaacatgt gaaaccctgt ctgtactaaa aatacaaaaa ttagccaggc |
| 117061 | atggtggtag gtgcctgtat cccagctact cgggaggctg aggcaggaga attgcttgaa |
| 117121 | cccagaaggt ggaggctgca gtgagccgag atcatgccac tgcactccag cctgggtgac |
| 117181 | agagcgagac tctgtctcaa aaataaataa ataaataaat aaaaggatac tgttatgtta |
| 117241 | agaattgctt ttaaggatat ttcataagta gctactgtct tttcagctca agtgtttgtt |
| 117301 | gattggccag gcgtggtagc tcatacctgt aatcccagca ctttgggagg ctgagtcagg |
| 117361 | cagatcactt aaggtcagcg tggccaaaat ggtgaaaccc catctttact aaaaataaat |
| 117421 | attaaaaaaa attagctggg cgtggtggca gtctcctgta atcccagcta atcaggaggc |
| 117481 | taaggcaaga gaatggctta aactcgggag gcagaggttg cagtgagcca agattgcact |
| 117541 | gctgcactcc aacctgagca acagagtggg actctgtgaa ggaaaaaaaa aaagtatttt |
| 117601 | ttgattgcct ttgagaggaa cggttgtata ttactcagat ttttaaaaaa ttgttctttt |
| 117661 | atggctgtat tctttaaggg attaaggaat gggcaatata agtgtatatg tttcaataaa |
| 117721 | aacgattagt gatcttctag tgagaacagt ttaaatctat atttagcaat tttttttaaa |
| 117781 | ttgtcaggta tggaagattt tagagcaacg taaagtccat gtagatttca ctggccttta |
| 117841 | tatttttttt aggcaagtta ggaaatcaac attgaatccc aatgcaaagg agttcaaccc |
| 117901 | acgttccttc tctcaggtag gtttattact ttctttgagg ttatctagtc ccaaaaaaag |
| 117961 | aaaaattatt agtaatagtc cttcttccat acctgccatc tgaattttgt tttagtgtgc |
| 118021 | tgaaccaacc ttctttcttt tttttacatg gccattaatg aatacttttt aaacattaaa |
| 118081 | aaaaggtctt tgttttgtca tcaattagat gtgatcttgg gcaaatcttt gaatttctct |
| 118141 | gacccagaat ttgacgatgg ttggctagct aggctgtcag gtttatagat acgtcctctg |
| 118201 | cacctgaggg ttttgcatca ctggattcaa ccaaccatgg atcaaaaaca tagttaggat |
| 118261 | aatctatact gaacacatgc agacgtttcc ttgtcattat tccaaaacaa tacagtaaag |
| 118321 | catttacctt gttttaggta ttataaataa tctagagatg atgtaaagta tataggagga |
| 118381 | tatgcatagg ttgtatgcga atactacatg attttatgta agggacttga gcattccaag |
| 118441 | actttggtat cttcacaggg tactgtaacc aatcccccac agatactaag agatgactgt |
| 118501 | actattgtta ttattcgact gagatcataa gaagatatat ttatttttaa tttttaaaaa |
| 118561 | cacttccatc agtttcttaa aaatagctgc cactgttttt aatatttttt aattgacaaa |
| 118621 | gttttaagtt cctactgaaa cattttttct tttattgaaa tgtgaaaatt tatgtgctgt |
| 118681 | gtttttgttt tcaataaaag ggacatagtt aaagcaagta aaattagaaa gactgggaaa |
| 118741 | atccgtcttt aaattgcaat aatagttcat ctgttacctt gagataattg aatttattgt |
| 118801 | tgtttttgta gccaaagcct tctactaccc caacttcacc tcggcctcaa gcacaaccta |
| 118861 | gcccatctat ggtgggtcat caacagccaa ctccagttta tactcagcct gtttgttttg |
| 118921 | caccaaatat gatgtatcca gtcccagtga gcccaggcgt gcaagtaagt catagaattt |
| 118981 | gatgttcact tagcctcccc aattgtttgt atctgacacc aagcactctt taggttttca |
| 119041 | gtgacttgag ggtgtgatgg ttatgcatat gcatttgaaa cagacaggca tgcagagatt |
| 119101 | cagtgtgttg ttaagtatga ggacctaaat ctgagaatgt tttctgtgaa aaagatggtt |
| 119161 | tagatttact gtagtttggg gtttgttcct tttagctgtg ggtatgatct aattttttaa |
| 119221 | tgactaatgg agaatcagga aaccttctca tgcctagctc tctagcaata taaaactaag |
| 119281 | agtgacagaa taccttgtta ttatcatagg tgcctaatgt taattttttt tttaattctc |
| 119341 | tcaagccttt atacccaata cctatgacgc ccatgccagt gaatcaagcc aagacatata |
| 119401 | gagcaggtaa aggtgagaat aatcctgcct gtgtttgctt gtagtttgca tgctgcatga |
| 119461 | attgagtaac taagtttata atgaataaat agttgtagtt tagctctgac tttttgatga |
| 119521 | ggctatgcat tggcttttga tgaacaacat tacatagata ttcacatgga ttttatgaag |
| 119581 | aaaaacaggg gagaaaaaat gcccatcagt tgtgattata tagtatcctc ttcaaaaaga |
| 119641 | gtaattggag gcctggtgtg atggctcaca cctgtaattt tagcactttg ggaggccaag |
| 119701 | gcaggaggat tgcttgagct caggagccca agatcagcct ggacaacaga gactttgtct |
| 119761 | ctactaaaat tcaaaaaaat tagctgggca tggtggcata tgcctgtagc cccagctgtt |
| 119821 | tgggggactg aggcgagagg atcacttgag cccaggaagt agaggctgca gtgagctgtg |
| 119881 | attatgccac tgccctccag cctgggcgac agagtgagac cccgtctcaa acataaatac |
| 119941 | tggctgggca tggtggctta tgcctgtaat cccagcactt tgggaggccg aggtgggtgt |
| 120001 | atcacctgag gtcagtagtt tgagaccagc ctggccaaca tggcgaaacc ccatctctac |
| 120061 | taaaatacaa aaattagccg gacatggtgg cacctgccgc ctgtaatccc agctactagg |
| 120121 | tggggctgag gcaggagaat tgcttgaacc cgggaggcag gggttgcagt gagccaagat |
| 120181 | cgtgccactg cacttcagcc tgggcaacag agtgagactc catctcaaaa caaacaaaca |
| 120241 | aacaaaaaac aaacaaacaa aaaaaccaga ctaattggct ggacacagtg gctccatgcc |
| 120301 | tgatatccca gctggaggat gacttgaacc catgagttcg agagcagcat gggcaatata |
| 120361 | gtgagaccct atctcaaaaa aaaaaaaaaa agttaattcc aaagcttttt gatctgaaat |
| 120421 | ctgatttaaa tctgaactta aatttgaaga agagggtttg ctagattaat ttactagatt |
| 120481 | gctaaccttg ctttatatat acctacagtt atttccccaa agccagaatt tcttttgaag |
| 120541 | cagaggggca actaacttca accaatgtta agatcctatt agaaggatgt ttcggctagg |
| 120601 | cttggtggct cacgtgtaat tccagcactt tgagaggctg aggtgggcag atcacatgac |
| 120661 | cgggagtttt aagaccagcc tgggcaacat ggcaaaaacc tatctctgca aaaaaaaaat |
| 120721 | agaaatctta gccagccgtc atggtgtgct cctgtagtcc tagctactcg ggagactgag |
| 120781 | gtgggaggat caattgaaac cagaaggtcc aggctgcagg gaactgtgac tgcaccactg |
| 120841 | ggctccagct tgggtgaaag agcgaaaccc tgcctcaaaa agaaaaataa gatggatgtt |
| 120901 | tctgcattaa aattagggag ttgtcgtata atgtagttgc ataaactagt attctgtgct |
| 120961 | tgtgtggtta aagagccttc gtagaaaaaa tcccacattt ttcttaaaag gaaatctttt |
| 121021 | ggccaggtgt ggtggctcac atctgtaagc ccaacactct gggaagccga ggtgggcaga |
| 121081 | tcacttgagg tcaggagtac aaaaccatcc tggccaacat ggtgaaaacc cgtctctact |
| 121141 | aaaaatacaa agatcagctg ggcatggtgg tgcgtgcctg ggtgacagag cgagactccg |
| 121201 | tccaaaaaaa aaaaaaaaaa aaaagagttc ttttaatgtt ggaaaatgct aaagggtttt |
| 121261 | ttttttgcca accagttaat ttagagtgat taactgctat cagttgagaa actatagaaa |
| 121321 | gtagaataat ttatacagaa aagacatttc tcagtgccca ataattgcct ttctgacata |
| 121381 | aagttttcat ttttcctgaa ttaataagat ttcctcaatg tgtttttttg ggtgttttgt |
| 121441 | gtgtgtgtgt gtgtgtgtgt gtgtgtgtgt gtgtgtgtgt gtatgtgttt gatacagggt |
| 121501 | cttgctttgc tgctgaggct ggaacgcagt ggcgctatca tggctcaatg cagccttgac |
| 121561 | ctcctgggct caagcgatcc tcccttctca gtcccctgga tagcgggggc tacaggtgca |
| 121621 | caccaccaca cctagctaat ttttgtattt tttgtagaga tgggttttgc catgttgcct |
| 121681 | aggctggtct caaactcctg ggctcaagcg atctgcctgg ctctgcttcc caaagtgcct |
| 121741 | gcgcccagcc aattttctcc atgtttgacc taattgtgat ttcatagatg ttaactaaaa |
| 121801 | ctcttaattt tcgttttctc agtatgctat tttttttttt tttagccttg gaacatatga |
| 121861 | acctgttgaa agaactctgc ctgaaataat gtaatcaaat tatagagttt aatcttattt |
| 121921 | tgagggcctt tagaaattct gagaagaaag tgggtttttt tttttactgc cattttaatg |
| 121981 | tagtgttaag gtgttcatgt atcaccagca ggtgtagctg ttttcaatga ttacttaaaa |
| 122041 | caatgcaatg ggaacttttt gttgtcatta aaatataaaa ggttactgta gtaagagcaa |
| 122101 | gcatgacagt ttggctatct gatgggagag tcacattcta acttcaggag gtactgtctt |
| 122161 | tttaatagaa atgatatact cagagtctgg gcacggtggc tcacgcctgt aatccagcac |
| 122221 | tttgggaggc cgaggtgggc agatcacgag gtcaggagat caagaccatc ctggctaata |
| 122281 | cagtgaaacc gtgtctctac taaaaataca aacaattagc tgagcgtggt ggcaggtgcc |
| 122341 | tatagtccca gctactcggg aggctgaggc aggagaatgg catgaacctg ggaggcagag |
| 122401 | ctggcagtga gctgagatgg tgccactgca ctccagcctg ggtgacagag cgagactccg |
| 122461 | tctcaaaaaa aaaaaaaaaa aaaaaatagt agagaaaggg ctttgccatg ttggccgggc |
| 122521 | tggtcttgaa ctcctggcct caagtgatcc acctccctcg gcctcccaaa gtgctgggat |
| 122581 | tacaggtgtg agccactgct cctggcctga atataccact tttacctatc atcagttgat |
| 122641 | gaacatttgg attatttcct ttttctggca atgagtaatg cttttgtgga ttttcatgta |
| 122701 | caaattttca tatgaggctg ggagcagtgg ctcatgccta taatcccagc agtctgggag |
| 122761 | gctgaggtgg gcagatgact tgaggtcagg agtttgagac cagcctggcc aacatggtga |
| 122821 | aatcccatct ctactaaaaa tacaaaaatt acactggcat ggtagcgtgc acctataatc |
| 122881 | ccagctattc aggaggctga ggcaggagca tcagaatcgc ttgaacctgg gaggcggagg |
| 122941 | ctgcagtgag ctgagatcac accactgcac cccagcctga gtgaaagagt gagtctcaaa |
| 123001 | aaataaaaaa taaaattttt tttcatgtgg ccttagattt tcatttctcc taaagtagaa |
| 123061 | atgctgtgat ggaactgcca aacttttcca aagcagctgc atcattttgt atttctacca |
| 123121 | gtaatgtaca agtgttccag tttctccaca tcctcataaa taaccgatat gtctttggtt |
| 123181 | tgggttatgt ccattctagt ggttatgaag tgtcattgtg gttttttgtt tttttgtatt |
| 123241 | gttttgagat cgtgcccagg ctggagcaca gtggcacaat ctcggctcac tgcagccttc |
| 123301 | gcttcctggg ttcaagcaat tctcctgcct caccctccca gatagctggg gctgcaggca |
| 123361 | tacgccacca caccaggcta atttttatat ttcttgtaga gatggagctt ctccgtgctt |
| 123421 | cccaggctgg tctcgaattc ctgagctcaa gcgatccccc tgcgtcagcc tccagagtag |
| 123481 | ctggggttat aggcgtgcac caccgcgctc ggcccatttt tgtattttta gtagagatgg |
| 123541 | aatttcacca tgttggccag gctggtcttg aactcctgac ctcaaatgat ccgcctgcct |
| 123601 | caccttccca aagtgctgag attttagacg cgaaccacca tgccctgact ataggttatc |
| 123661 | tttttacttg cttgatggtg ttctttgtaa cacagttttt aattttgatg aagttcaatt |
| 123721 | tatctgtttg ttttttcttt tgttgctgtt gctcctgatg tcatatcaga caaagcattg |
| 123781 | cctaactcaa ggccacagag atttactcct atgaaacgcc tataaaactc ctatgatttt |
| 123841 | tatagtttag ctcttaacat ttaagtctac aatctctttt gagttaattt ttgtgtatga |
| 123901 | gatgagagta gtggtccagg tttttccttt tgcttgtgga tatccgttgt ccccacctca |
| 123961 | tttgttgaaa agactattct ttcctcttaa attgtttgtt tgtttattta tttttgagat |
| 124021 | ggagtgtcgc tctgatggag tggcgctaac ttagcttcac tgcaacctcc gcctctcaga |
| 124081 | ttcaagcgat tcccctgcct cagcctcctg agtagctgga attacagggg tgcgccacca |
| 124141 | cacccagcta atttttgtat ttttagtaga gacggggttt taccgtgttg gtcaggctgg |
| 124201 | tctcgaactc ctgatctcgt gatctgcctg tctcctggca ccctgggagg ctgagaggct |
| 124261 | gaggtgggag gatcacttga gctcaggagt ttgagaccag cctgtaccat tatgcctggc |
| 124321 | taattttaga atttatctta aagtataaaa tgtgaatcca atttatcttg ttctaaatga |
| 124381 | ctatccaaaa tgttttaacc agttttatta gtctgtaatt tacatacaag aaaatgctca |
| 124441 | tctttttatg tttacatttt aatgagtttt gacaaatata tttgctcatg taactacttg |
| 124501 | cttcatcagt gaagatggaa aacattgtgc ctgttcctct tctctgtcca actgtacttt |
| 124561 | attaccacta gctccagtta accagtaatc tgccttcttt tactatagat tagatttatc |
| 124621 | ctctttagat ttctttttct tttttttttt ttgattaggt tttttttttt ctttttttac |
| 124681 | gtaaaaaaat ctttttttgg agacgtctca ttatattgcc caggttggtc tcgaactctt |
| 124741 | gagctcacct cagcctccca gagtgctagg attacagatg tgagccacct cagccagccc |
| 124801 | ctagattttt tttttttttt taataaatgg aatcaaacag cgtgtaacag aggtgttcaa |
| 124861 | tcttttggct tccctgggtc atattggaag aagaattgtg ttgggccaca cataaaatac |
| 124921 | agtaacacta atgatagctg atgaacaaaa caaaaaaaaa tagcaaaact tataatgttt |
| 124981 | taagaaagtt tatgaatttg tgttgggcca cattcaaagc cgtcccagga cgcaagttgg |
| 125041 | acaagcttgg tatataattt catatgtgtg tcctaaacag tgtagtaatt tgaatttcat |
| 125101 | gttagtatca gcttattcct ttttgtttgt ttgtttgttt ttgagatgga gtcttgttct |
| 125161 | gtgtcccaga attggtctgc aattccactg cctcagcctc ccaagtagct gggattacag |
| 125221 | gcacgtgcca ccacacctgg ctaatttttg tctctctctc tttttttttt tttttttttt |
| 125281 | ttttttagca gagacgggat ttcaccatgt tggccaggct ggtctcaaac tcctgacccc |
| 125341 | aaatgatcca cctgccttgg cctcccaaag tgctgggatt acaggtgtga gtcaccgtgc |
| 125401 | ccagccagct tattcctttt tattgctggg tagcatttca ttttatgatt ataccacagt |
| 125461 | taatttaccc attactagtc gatgggcatt tgagttattg ccagcttttg gctattatga |
| 125521 | atgaagctgc tgtgagcatt tgtgtacaag tgcttgtgtt tttatttctt ttagttaaat |
| 125581 | acctagaatt ggaattgctg aggtatggta agtgcatatt tcattttttt aaaaaattta |
| 125641 | ttttattttt tatttattta ttttttttga gatgaagtct cactctgttg cccaggctgg |
| 125701 | agttcagtgg cgtgatttca gctcatggca acctccctgt cccgggttca agcaattctc |
| 125761 | ccgcctcagc ctcccaagta gctgggatta caggcgcgca ccaccatgcc tggctaattt |
| 125821 | ttttgtattt ttagtagaga cggggtttca ccacgttggc caggctggtc tcgaactcct |
| 125881 | gaccacaagt gatccacccg ccccagcctc ccaaagtgtt gggattacag atgtgagcca |
| 125941 | ccacacactg cctggtaaat acatatttca attaataaga aactagcaat cttctaaagt |
| 126001 | gattgtgtca ttttacattc caactgatca ggtacatgtg taggttccat gtgttctgca |
| 126061 | tccttgccaa cacttggtat tgtgttatct ttttaatttc aacaggtcta atgggtgtct |
| 126121 | tatggtatct cattgtgatc ttaaatgtac atttctctga tgatgactga tccaggagca |
| 126181 | cctcatcatg tgtgtgtttg ttttcagctg tcaacctttt tttagtaaat ggttcaaatc |
| 126241 | ttttttccat tttatttatt tatttattta tttgatggaa tctcactcta ttgcccaggc |
| 126301 | tggaacgcag tggtgccatc ttggctcact gcaacctccg cctcccaggt tcaagcaatt |
| 126361 | cttacgcctt agcctcccaa gtagctggga ttacaggcat gcgccaccat gcctggctaa |
| 126421 | ttttgtattt ttagtgtagg tggggtttca ccatgttggt catgctggtc tctaactcct |
| 126481 | gacctcaggt gatctacctg cctcggcctc ccaaagtgct gagattacag gtgtgagcca |
| 126541 | ctgcgcctgt cctaataatt tctttttgtc tcaatgtttc tgcctgggtg cactggctca |
| 126601 | cgcctgtaat tccagcactt tgggaggcca acctggatgg atcatttgag ccaacagttt |
| 126661 | gagaccagcc tgaggaacat gacaaaaccc tgcctttgca aaaaaaaaaa agaaaaaaga |
| 126721 | aaaattagcc aggcacagaa gcgcattcct atggtcccag ctacttgggg ggctgaggtg |
| 126781 | ggacaatcgc ttgagcgagg ttgcgggggt ttggagggcg atggaggggt gatcgaggtt |
| 126841 | gcagtgagct gagattgcac tactgcactc cagcctgggc aatagagcca gaccctgtct |
| 126901 | cacaaaaaaa agaaaaaaaa gtcatgtttc ttttcttact gtgaaaataa agttactact |
| 126961 | tttagtaaat tattttaagt tatttatata ttctggttac aagtcctttc tcagaatatt |
| 127021 | gtgaatattt tctcccagtc tgcggttttt tttgaagagc cagtattgct aattttaatg |
| 127081 | aagccttatt tatcaagctt ttctcttaag gttcatgctt ttttgtatca taataagaaa |
| 127141 | tcttttacgt accctaggtt atgaatgttt ttatggttag gtatatggct gatttcaggt |
| 127201 | taggttttgt gtagggtgtg atgtaaaggt ctagcttcat tttctccacc ataaatattt |
| 127261 | actcggtttc tctggcacca gcctctgttt tccattggtg gctttatttt ttttctgttc |
| 127321 | ttgaaacaag agtctcgatc ttgttaccca ggctggagtg cagtagtgtg accttggctc |
| 127381 | actgcaacct ccacttccca gggtcaagcg attctgcctc agcctctcga gtagctagga |
| 127441 | ttacaggtgc ccgccactac acccagctaa ttcgtatttt tttttttttt ttttttagta |
| 127501 | gagacagggt ctcaccatgt tggccaggct agtctcgaac tcctgacctc aggtgatctg |
| 127561 | ctcatctcag cctcccaaag ttctgggatt acaggcatga gccactgcgc ccagccatag |
| 127621 | tagctttatt gaattcagtt gactgtattg tatgtgtgtc tatttgtgaa ctgttttgtt |
| 127681 | gtattgatct ttgtatatat ccttatgcca attctctctt tattgctgtt actttgtaac |
| 127741 | caacctttaa gttcatatga gtctcccagt tttattctcg tcaaaattac tcttattctg |
| 127801 | cgttctttga atttgcaaat aaattttaga atcagcttgg gattgtgcac tgaatcttta |
| 127861 | tatcagttct gggagaaata tcttaacaat atggaatctt cattgaggtc atcatatact |
| 127921 | gctccattta tttaagtctt aagtttcacc agtgttttct agttttcttt gtatcagttt |
| 127981 | tgtgcctgct ttcttaaatt tatcccttaa tatttcatct gttttgtgct gttgtgagtt |
| 128041 | atattttaaa aactttcaac gtttgtttat tcgtaaatag agatgcacct gatttttgaa |
| 128101 | tattgacctt gtgtcttgat gtgttggtaa acccactgtt tctggcagcc ctttaagact |
| 128161 | taaacataca atcatgatct aatcaccatg ttggtgtttt tgggtttttt ttttttgtct |
| 128221 | tattgtactg gtgcattact gaaaaaggca tgagattttg ccatgctccc atttttaggg |
| 128281 | gtgagacatt gtctttcact attaagcata cagttaggtg ttacttcagt tcctaatttg |
| 128341 | cagaggtggg tttgttttct ttttaatcat gaatggttgt tggattatgt tcaaatactt |
| 128401 | atcatctact aagtatatca tattgaccag gaacagtggc tcatacctgt aacctcagag |
| 128461 | ctttgggagg ccaaggcagg aggatcgctt gaggccagga gttcaagacc aacctgggtg |
| 128521 | atgtaggaaa accccatatc tacaaaacaa tttaaaaatt tgctgggtgt ggtggcacac |
| 128581 | acctgtagtc ctaactactt gagaggctga ggaaggagaa ttgcttgagc ccagtagttt |
| 128641 | aaagcagcag tgagctgtga ttgtaccact gtactccagc ctgggtgaca gaaggagacc |
| 128701 | ctgtatttaa agtgtgtgtg tatgcgtgcg catagatgga tagataataa tgtaattcca |
| 128761 | ttatggtcat acaaactgat atgaaatgcc attttatcat ataacaagtg tctttttgtg |
| 128821 | gttgaatttg tttctggatt tttcactctg cttcactaat ctaataggac taccttctca |
| 128881 | tccactcact gccaacattg attttttttt tcagattacc ttgaattttc tgtttatttt |
| 128941 | tccatatgaa ctctataatt aacttactac taaaaaaatc agttgccttt ttaaaaccaa |
| 129001 | ctgatcttta aaatatatct tggctgggcc cggtggcagg cacctgtaat tctagctact |
| 129061 | tgggagactg aggcagaaga attgcttgaa cccaggaggc ggaagttgta gttgagttga |
| 129121 | gattgcgcac ctgtactcca gcctgggtga cagagcaaga ttccctctta aaaaaaaaaa |
| 129181 | aaaaaaaaag aaacagaaaa gataaatctt tttacaataa tttgttccaa ttagggtcca |
| 129241 | agtcaggctt gcaatttgga tttgtttata tgctgaagtc tttttttttt tttaattgtt |
| 129301 | tcatattgtg gtaacttttt tttttttttt ttgagatgga atcttggctc tgtcacctag |
| 129361 | gctggagtac agtggcacaa tctcaactca ctgcaacctc cccctctggg gttcaagcaa |
| 129421 | ttctcctgcc tcagcctccc aagtagccca gccttttttt tttgagacag agtctcgctc |
| 129481 | tgttgcccag gctggagtgc agtgatgcga tctcggctca ctgcaagctc cgcctcttgg |
| 129541 | gttcatgcca ttctcctgcc tcagcctcct gagtagctgg gactacattc gcccgccacc |
| 129601 | acacccggct aatttttttg tatttttagt agagacaggg tttcaccgtg ttagccagga |
| 129661 | tggtatcgat ctcctgacct cgtgatccgc ccgcctcggc ctcccaaagt gctgggatta |
| 129721 | caggtgtgag ccactgcgcc cggccttgta tttttaatag agatggggct tcaccatgtt |
| 129781 | ggccagcccg gtcttgaact cctgacctca aatgatccac ccgcctcggc ctcccaaagt |
| 129841 | gctgggatta caggtgtgag ccatcgctct cagccttgcg gtaacttttt attacgaatg |
| 129901 | tattgagaca ttaataacct aggccagtca tgtttcatcc ctacccatcg tctcttaaaa |
| 129961 | gctttgagtc cactggatta ttctgaagca aattctagac attgcatcag tttatccacc |
| 130021 | aacattttag tgtgtatctt taagttggtt ttggttttgt tttttgttct tgagatgggg |
| 130081 | tctggctttg ttgcccaggc ttggagtgca gtagtgcaat catagctcac tgctgctgcg |
| 130141 | aattcctggt ctcaaaggat cctccctcct cagcctctca agtaactgtg actacaggca |
| 130201 | catgccacct tgccagcttt tcttttcttg tcttgtcttt cttcttcttt gtttttttgt |
| 130261 | ttgttttttg tttttttttg agacagagtc tcaccatctt tctatcttgc ccaggctagt |
| 130321 | cctaaattcc agggcttaag ttatctttct acctcagcct cctaaagtgc taggattaca |
| 130381 | ggccagcact ttaggaggtg ctggatgagc catcacaccc agccaagtca taggtttttt |
| 130441 | tgtttgtttg ttttttgaga cagtgtctaa ctctgtcacc caagctggag tgcagtggca |
| 130501 | tgatttcagc tcagtgcagt ctctaccaat tgggcttagg tggtcctccc acctcaacct |
| 130561 | cccaagtagc tgggactaaa ggtgcgcgcc accatacctg gctaattttt gtattttttg |
| 130621 | tagagacagg gtttcgaatt cctgagctca agcagtctgc ctgccttgac tcccaaggtg |
| 130681 | ccaggattac aggcatgagc cactgcactc agccctcaca gttttaatta cagtttttcc |
| 130741 | cttagttttt gtcttgttca tatccagctt gtcttgtatt tttttcccac gatctgaatt |
| 130801 | ttgctgactg tatccctgtg ttgatattta aagtagactt ctgtcccccg taatctttgt |
| 130861 | aaactgatag taaataatga aggcttgatc agattgggtt tttttttttt ttccccaatg |
| 130921 | tttcacagat gtgtgtactt tcagtgagga gtcatgtaat cagtcttttt cctgatagga |
| 130981 | gtagtcagtg agttcctaga tgttttatct atccaggaga taatatgtcc ctttagcgcc |
| 131041 | ttaatttttt tggtgtgttt tttagcagcc attgatgata attgtctagc ccaagatcag |
| 131101 | ttatttcctt aggggttgta aaatggtgac attcttttcc tttcatccct tcttcaatta |
| 131161 | ttgcctggaa tatttctata aagaaaaact ttcccatatc cagctgttcg gttaccctga |
| 131221 | ggtatagctt tcttaggaaa agtaatttaa aatgttaatc atttcccttt ttaaggcagt |
| 131281 | cttcaaaata atgagttggt tttctgttat cctccaaagg taaccagtga ggtggttttt |
| 131341 | ttgtcgttgg ttcttactat cagtataaac ttctggaatt tttttttttt ttttaatttt |
| 131401 | ttggagacaa ggtctggctc tgttacctag gctggagtgc agtgggatga tctgggcata |
| 131461 | ctgcagcctc aacttcccga gctaaggcaa tccccccacc tcagcctccc aagtagctgg |
| 131521 | gactacaggc aagcaccacc gtgcctggct taatttttgt atattttgca gagacagggt |
| 131581 | ttcaccatgt tgcccaggct ggtgtcgaac tcctgagctc aagcagtctg cctgtgtcag |
| 131641 | cctcacaaag tggtgggact acaggcatga gccaccatgg caggccagaa tcacaataaa |
| 131701 | cttataaatt aacttgagaa gaaatgattg atgtcttcat gatgttgagt cttcctgttc |
| 131761 | aagaacaaag tataccttca atagcatatt aaagtttatc cttggctgga tgcagtggct |
| 131821 | gacgcctgta atcccacctc tttgggaggc agaggtgggc agatcacctg aggtctggag |
| 131881 | ttcgagacca gcctggccaa catggtgaaa ccccgtctct actaaaaata ttttaaaaaa |
| 131941 | agtattagct gggtgtggtg tgcacctgta gtcccagcta ctctggaggc tgaggtagga |
| 132001 | gaatcgcttg aacccaggag gcagagagtg cagtgagtca agattgcacc actgcactcc |
| 132061 | agcttgggca accgagcgac actctgtctc aaagaaaata aataaataaa aataaagttt |
| 132121 | atctttaagg ttttgtacat ttttttcagt gtatgcctta ggtaggttct tttttaatgt |
| 132181 | tagtgtaacc cagggacttc tcttccattg catcttctaa gtaattactt atgaagtacc |
| 132241 | atatatgaag gctattgctg tttatatgtt agtttttacc ctgctcctct actaaattcc |
| 132301 | aatcctttga ggtattggat aaaaatattt ttagcatttt tcaaataaca ggcagagtca |
| 132361 | agggcttggt ttcttttctt cccctcctgt cccctaccct cccctttttt gagacagggt |
| 132421 | ctcacttctt cgccgaggct ggagtgcagt ggtgcagtta cggcttaccg cggcatctgc |
| 132481 | ctccctggct gaaaagttcc tcccacctca gcctcctgag tagctgggac catagatgca |
| 132541 | cagcaccgca gctggctaat atttttgtat tttttgtgga ggcagtgtct ccccatgttg |
| 132601 | cccagggtgg tcccaaactc atgagctcaa gcagtccgct cgccctggcc tcctaaagtg |
| 132661 | tagggattat aagcgtgagc cactgcgcct ggcctgggga tcatgtttta acatgagaat |
| 132721 | tagtggagac aaacacatga tatctaaata atagcaccat agtatacttg actagctttt |
| 132781 | taattatttt ttaaatatac aggaaggtaa taagtaacaa agtaataata gtgaatagtt |
| 132841 | taagctcagt tagcataatc gggcaaactt tcatttgata aaagtgataa gtagttttca |
| 132901 | gtggcttttt tgtttaccag aaggaggtgg tttttaaata cgtgcatcca agataaaata |
| 132961 | taaaaaaatg ttcaggtttg ctttcctaca tagataaaat aatatgtaac tagctctccc |
| 133021 | aaatttcagc aacagttagt gaatgtttag ccacaaattt gcagttaatt atataatcag |
| 133081 | ttcttaggat tttatgaaca agttctatat tctttgtgcc ttatacctag ttgtaagcag |
| 133141 | tcattccaca attattttcc tgaagtggct tggttaatgc cacaccagaa acaggtcaca |
| 133201 | gacaatagtg ctgtaagaaa tgtgtgagga aagaggcaca tgggaagtag ctagctcgtg |
| 133261 | ctggaggaac tggaaaaaaa cctcacatgg gagatgacag ttgagctgaa ttcttaacta |
| 133321 | gagttgtaac agggcgaggc ccttacatgc agaccacctg tgtggattaa gataagacat |
| 133381 | aaagtaatct tttaaaagaa ctattattta gaaacctggt atatgctaca tggtgctgtg |
| 133441 | ttatactggg tttgagaaag aatgggaagt gttacaagga ttcagtggct ggaaattaag |
| 133501 | gaagatagaa agttagtgtt ggatctgttt tggctctttg gtcatgcctt tgtttttctc |
| 133561 | aaaatgaatg cagtgcccgt cccagaaaat accatatgag aagcgatttc ataatgctgt |
| 133621 | gagagtctgt tacagggact tgatcaagtc tgagggccat gagagaaagt ccctctgagg |
| 133681 | aagttgcttt caagctgaca cctgaaggat gaagcagaat tatcccagct gggatttggg |
| 133741 | aactggtgtt tgaggctgag gactagcatg catgatagga aaataaccca gagtggcaga |
| 133801 | agtgggagtg gtatgagatg gcatcagaga cgcagattca gggtcaaatc attcagagcc |
| 133861 | tcctagacca tgtgaacaca tgtattatgc tgtggagata ctgtttaata ggcagtctgc |
| 133921 | ttttttttct gcagtaccaa atatgcccca acagcggcaa gaccagcatc atcagagtgc |
| 133981 | catgatgcac ccagcgtcag cagcgggccc accgattgca gccaccccac cagcttactc |
| 134041 | cacgcaatat gttgcctaca gtcctcagca gttcccaaat cagccccttg ttcagcatgt |
| 134101 | gccacattat cagtctcagg taaggctggt aaggcctaac tcttaatttt tgtaccatat |
| 134161 | aaaaaaactt ttaatatggt aaagggattt tcctttataa tttttgctct tgtgtgatgg |
| 134221 | tagggtagat agctaaggac ttggggaccc ttttcaatat atattcgaag gttactgatg |
| 134281 | attgtaagag gttcagagga aacagccaag aaagatttga gagtttacag ctgtttctgg |
| 134341 | aaatctggaa accatggagt taaaaatctt aactaaagtc tgcttggctc tatttgcagt |
| 134401 | gttaatgtgc tttctttatt ttttgtttga acacagcatc ctcatgtcta tagtcctgta |
| 134461 | atacagggta atgctagaat gatggcacca ccaacacacg cccagcctgg tttagtatct |
| 134521 | tcttcagcaa ctcagtacgg ggctcatgag cagacgcatg cgatgtatgg taggaagcac |
| 134581 | tttgtttgtc tcttccagtg tgtgtgactc ttcttaattt aagtttctga aaacatactc |
| 134641 | tatctaagaa taacctgacc ttttatgaca ttgagggtca agaatctgaa ggaaaagatg |
| 134701 | aacccatttc tttgcctgac ttgctttata acttttggca aatagtttct acttctgtac |
| 134761 | ctggtcttca gatctctttc ctgctttaac taaaatgtaa tgatgtatat aatggcaaag |
| 134821 | catctttgtg gagaaaggta cctttctcct cttcctcatc aatattatgc tttggtatat |
| 134881 | cctgcctacg acatgcaaga gaattttata ataataaaag cataaaggtg ttctccagca |
| 134941 | tgaaaacatt ttgcttcact acttgatctg agggtcactg gcattacata ttttttttgc |
| 135001 | tgtttgttat aatgataata ctatgtttct acatcatgct gtattttaat ggttgaatat |
| 135061 | tatgtcatat tagatatatt ttagacatga gtcacacttt aaatataacc aatgtgaaca |
| 135121 | gaatgctgaa atgaaaatga gaagtatttt atgtaaaact aagcagtatt tatatgtgag |
| 135181 | aataataagc aaaaaaaccc atcttcgttt tgtgactaaa cagagaaatt tgtgtagatc |
| 135241 | aacttagcag ctgtctaaag taccaaaata atagattttt cactgttgat aatttaaaat |
| 135301 | aaaatgtcca tttgtatatc ttatgataca gaattaatgg attgcttcaa atgtttttca |
| 135361 | gaatatgttt ttaaatagta ctgatttcat taagatgttt tgttctgaat atttctgaga |
| 135421 | actaccgtag tgtcgtttag ttttcctatt tgcgtttttg gttgtttgga gtaggggata |
| 135481 | attttggttt attcatacag ttgaaaagtg tactgctatg agaatgagat tatggttaca |
| 135541 | tgtaactaca tgggcatttc atttttaaag cctctttgaa ctttttgaaa tactaagaat |
| 135601 | ataaaatttt tattttttaa gtttagatgt cctgaacgag tatgtttagg caaaattgag |
| 135661 | ttatttaaga atttataggc tgggcgcagt ggctcacgcc tgtaatccca gcactttggg |
| 135721 | aggccaaggc tggcggatca tgaggtcagg agatcgagac cagcctggcc aacatggtga |
| 135781 | aaccccatct ctactaaaaa tccaaaaaat tggccgggtg tggtggcatg tgcctgtagt |
| 135841 | cccggctact tcggaggctg aggcaacaga attgcttgaa cccgggaggc agaggttgca |
| 135901 | gtgagccgag atcgcgccac tacactctag cctgagcgac agagtgagac tccatctcca |
| 135961 | aaaaaaaaaa aaaaaaaaaa gaatttacag atttctggca aaccttcttc ttgagacatt |
| 136021 | actacttttc ataccacctc tgtccttttt gaagaataaa agttttaaca ttccgtaggt |
| 136081 | taatgagaat aggacttggg cagcagcaat catccttcct gtcacctgta acccacagct |
| 136141 | tatgctttct tcctggaggt tcttgtctgc cacaaaggct cactgctgat aggaatttgt |
| 136201 | atatgatcaa aggtgtttag ttttataaaa cagttaagtc cagtcttaat tttccacatt |
| 136261 | atcactttca attttgtatt gtggattacg cattttaaat aaaaaattgt gtgattgcta |
| 136321 | cattttggaa aacatttttt tcaagaggcc catccgtaat ttaattgtaa aagatactga |
| 136381 | caaactaact tggtttatta ttttggttat gaccccgtca tttgacttgt ctttagttgt |
| 136441 | cttaacgggg actgaatatg cgtgcaaagg cacgattgat ttatcatgct ggcttttatg |
| 136501 | caacttgtat atattttaac aattttcctg tttgctaaag gcttaggtta aaagttcatt |
| 136561 | atgattgttt atacatttct ggtgaataca tcatgattta acaagtggaa agaacatctc |
| 136621 | tttccttcca ttttctggca tactcccctt ggaatcagat ctgaaacttt taagctaaaa |
| 136681 | tttccattgc atttggagag tagttatttg tgtatgcatg cttttgagac attgtagcaa |
| 136741 | taatactgta atgttgagcc gaatctttct cctcattgtg ttcattcact gccaacatct |
| 136801 | ggcttcatct tttggatgaa tgttcattgg ttctgaaaca gcctataggg taaatactgt |
| 136861 | gtttgaggta cagatgattt tcataactac ttcctagaac atgtccattt gaagagcagt |
| 136921 | ggggccttag accccaaagt ccatttatgt gtgggcaaat aggaaatgtt gcaaacaaaa |
| 136981 | caaagcacta gatctaatgt ccagtgaaat ctggaatgaa ctagtcatta gagccggttc |
| 137041 | tttcatgcca ggaaaaagtt actcagccaa atctgaacta ctctcctgca gtttacacag |
| 137101 | gtggtattta attgctgtct gtatggaggc aggctaggag caaggctgtg gacttgttgt |
| 137161 | gattgtcact agttaatcaa gattcccttt gtggtgctta agaccctaaa aaggacacta |
| 137221 | ggagctgggc atggtggctg acacctgtaa tccaagaact tggggaggct gaagtggagg |
| 137281 | atcgcttagc ccaggtgttc aagaccagtc taggcaagat ggcgagatcc catctctacc |
| 137341 | aaaaaaaaaa aaaaaaaaaa aaaaaaaaag cccagtcatg gtggcacatg cctgtagtcc |
| 137401 | cacctacaca ggaagctgag atgggaggat cacttgagtc caggactttg aggctacagt |
| 137461 | gagctatcat ggcaccactg taatccagcc tgggtgacag agcaagaccc tgtctctatt |
| 137521 | taaaaaaaag aaaacataag aaagaattgt ttcgttctat gccatcataa gccataattt |
| 137581 | aatctgctta agcatgttct tcattaaatc tgcagtgatt tatttgaatt attagacttt |
| 137641 | caaagcctta ttatatcaaa tataaacaaa atttgaagta cattcttata aactacaaca |
| 137701 | aacttacata gaagtgttaa ttttatactc atcttccctg aacaatttat attttataaa |
| 137761 | tatattaaat atattgtcat aaattttctc aaaggaacca aatactttga gtatgaattg |
| 137821 | tgcttttctt tttaagctac atcatatcta ggtttttaaa acatttaatg caaacagaag |
| 137881 | aacatgcacc cagatgttgg tgacaatttt atgtcacctt ttctcattca ttaattgtta |
| 137941 | tagccatagc caaaggcatt gaaaacatag gaccactaat gactgcaaaa tgaaatcctg |
| 138001 | attattgttt ttaaattttt agtatgttta atacacatat gctaacatta ctgaacagtt |
| 138061 | aaatgataaa ataggataat tattttattc taaaaaagta ttgaccttga cctctttcta |
| 138121 | gctatcttag aaagggcttt tgtcaaaaac cttatctctt tgatgtctct ttttttgaga |
| 138181 | tggagtctct ccctgtcgcc caggctggag tgcagtggcg tgatctcagc tcactgcacg |
| 138241 | ctccgcctcc tgcgttcacg ccattctcct acctcagcct cccgagtagc taggactaca |
| 138301 | ggcgcccgcc accatgcccg gctaattttt tgtattttgt ttagtagaga tggggtttca |
| 138361 | ctgtgttagc caggatggtc ttgatctcct gacctcgtga tccgcctgcc tcagcctccc |
| 138421 | aaagtgctgg gattacaggc gtgagccact gtgcccagcc tctttttttt tttttatttt |
| 138481 | ttatttattt tttatttttt ttttaatttt tgagaaggag tctccctctg ccacccaggc |
| 138541 | tggagtgcag tggcgcgatc tcagctccct gcaaactccg cctcctgggt tcaagcagtt |
| 138601 | ctcctgcctc agcctcctga gtagctggga ctacaggtgc ccgccaccac acctggctaa |
| 138661 | tttttgtgtt tttagtagag acagggtttc accatgttgg tcaggctggt cttgaattcc |
| 138721 | cgacctcagg tgatccaccc acctcagcct cccaaagtgc tgggattaca ggcgtgagcc |
| 138781 | actgccccgg cctctttgat gtctcttaat ctaacttcca tcattgcctc taccccatcc |
| 138841 | cttctaagaa gttactttaa ttttttttcc tctcacatct actctttttt tttttttttt |
| 138901 | tttttttttg aggtagtctc actctgtcac ccattctgaa gtgcagcggt gcgatctcag |
| 138961 | ctcactgcaa catctgcctc ccaggttcaa gcggtttttc tgcctcagcc tcccgagtag |
| 139021 | gtgggactac aggtgtgcgc caccacgacc ggccaatttt tgtattttta gtagagacgg |
| 139081 | ggtttcaccg tcttggccag gctgatctcg aacttctgac cttgtgattt gtctgcctag |
| 139141 | gcctcccaaa gtgctgggat tacagatgtg agccaccacg cccagcctca catctactct |
| 139201 | tctaatccat ctaattttgt tttatggtga tgcttttacc tttcagaaac agtaataata |
| 139261 | caacttttcc gactaactag agccattagg aagaattaga tccagaatcc ttttttgatt |
| 139321 | tgtttttggt agtttaatgc agataagtaa gaaaatatag ttaagttaaa aaaaaaaaaa |
| 139381 | atgaaaagca tccataatcc ctccacctga caactgcctt ttaacatttt gatgtgtatc |
| 139441 | cttccaggtg tatttaaata cactcaaata ccctacccct ttatgtagac atgttttaat |
| 139501 | aagaaataat attcatgttt atattcttgc tatgatccta aatttttgga tccattacta |
| 139561 | gataatcttt caggataatg acatttccat tagtaatgtt tttgcaaaat tgtgtgtcta |
| 139621 | ttgaattaaa cttgtaaaat agttttattt tggtacatga tttatatcaa ggttgttcag |
| 139681 | tagaatgcca tgttggtgtt tttattagat aatgatttta ttccttttac ttttaagcaa |
| 139741 | gtcagcatga caacttgaca cctaagtaca gaagaacagt gtcttccggt ttagtccttt |
| 139801 | cttttaaaat tctgtagcag tgtttaaagt gcttgtcatc tcttatgaaa atgaattatg |
| 139861 | catgaataca aaaagaaatt actaatatgt caacctttcc agaaaattcg gaaaatgcac |
| 139921 | acctcaaaag gctaatttac ctttctattt cccaaattca gcatgtccca aattaccata |
| 139981 | caacaaggag acaagccctt ctttctactt tgccagtgag ttgggttttt tatactaatt |
| 140041 | tttaattgta cagtaaaaca ctttttaaag gatacatgtt aagggagtag acttgttgaa |
| 140101 | caatattttc cttgtgccag tcaaattatt gaaagtactt atatatataa ataattcagt |
| 140161 | ttttaaaatg gaaataccca atttaagaag gctggagtta atgaaaaatg gagttgtttc |
| 140221 | agaaatcaat ttttgcatac caagcaaatg tgactgggaa atgcctaata ttttccttgt |
| 140281 | tagagaaact tcctaaacag ctttatacac acacacacac acacacacac acacacacac |
| 140341 | aaacacacac acccaagcca caagcttggt ataaatttaa aatgtttatt tatacacaca |
| 140401 | cacacacaca cacacacaca cacacacacc ccaagccaca agcttggtat aaacttaaaa |
| 140461 | tgtttattta tattctgata agatgaaatt tatgcctacc aggattttta attgaatagg |
| 140521 | attgatgaaa tactaaggga aaaacttttc agtcctgtgc atggctaaag gtttaaaata |
| 140581 | ctcaggaagg gccaggcacg gtggctcaca cctgtaatcc cagtgctttg ggaggctgag |
| 140641 | gcgggtggat catctgaggt cagcagttca agaccagcct agccaacatg gtaaaactcc |
| 140701 | atctctacta aaaaatacaa aaatcagcca tgcatgctgg catgcgccta taatctcagc |
| 140761 | tactagggag gctgagacag gagaattgct tgaacttggg aggcagaggt tgcagtgagc |
| 140821 | cgaagtcgtg ccactccact ccagcctggg tggcagagcg aaattctgtc tcaaaaaata |
| 140881 | aaatattcag gaagcagacc cctcaggata tcttgagctt aagcaagaga tcatgacctc |
| 140941 | tcaggtcatt atcttggaca gcacaggtcc cctctcccca cctggcaaaa agtacagaaa |
| 141001 | tagttgctcc ttcatggaga aagtctgggc agagctttct tctggaaatg aacttttaag |
| 141061 | gtacattttt cctatttgta gggcaatttg taaaaataag ggccggacgt ggtggctcac |
| 141121 | gcctgtaatc ccagtacttt gggaggccga ggtgggtgga ttgcttgagg ccaggagttc |
| 141181 | gagaacagcc tggccaacat ggtgaaaccc tatctctacc aaagcatggt ggcacgcacc |
| 141241 | tgtagtccca gctacttggg aggcggaggc acaagagttc catgaaccct ggaggtggag |
| 141301 | gttgcagtga gctgagattg taccactgca ctcaggcctg ggcaacagag agagactctg |
| 141361 | tctcaaaata aaaaataaaa ataaggctag tcttggactt tggtatttaa ataggaagga |
| 141421 | gtactaatat ttgtagaaat cctttagaaa tttgtgccat taatattgtc accttgtatg |
| 141481 | aaatgttgtg ttctagagga tattaaggat tcaaatttta tgttaggcac attttgagtt |
| 141541 | attttggggt gactcaatgt ctgactctac taaatgccat attagcattt aaaatgcatt |
| 141601 | tgaccttaaa tctttgttaa ttatgccatg acttggtatc caaaaataag ctgatacata |
| 141661 | catacataca tatatgtgtg tgtgtgtgtg tgtgtgtgta tatatatata tatgtatgtg |
| 141721 | tgtatatata atttatttgg tgctaggaaa tgttaaattt aatcctttaa tagatgctct |
| 141781 | ttaaaaagga gtcttgctgt atgtatatac tattaaaggg gaaactatgt ctgtgattgt |
| 141841 | agtgtgtaaa agatagtagg tgattttatt atgtactcaa tttgaggtct caaatgtagt |
| 141901 | tatcctcacc atcttactgt ctctgttagt agtttggtgt tgttttcctg gtaagtagct |
| 141961 | aaggtcctta atcattaaca cctaagcctt aattgcctta gcacaacttc ccctaaaagg |
| 142021 | gagtatcagt actttttaaa agaaactaac agttgggctg ctaatttaat ctgctgcttc |
| 142081 | atttccccct gttctaagcc attttatgat ggtttggtca agttgccttt tattcccctt |
| 142141 | ttagagtttt caactttcct tcacttccct ttttctgaat ttaacatcag atttacaagt |
| 142201 | tggaagattt tgttttgttt tataagtttt gcaatgctgg tgatctctta tgacttgtgc |
| 142261 | atccaaagtc aaaatgacaa aacctagtta caaattaaac acacagctct ctgtacttaa |
| 142321 | tttgcttcag tgagatcaca gctgaggaaa ctagttctgg aatgtggtta gtgttattaa |
| 142381 | ggatttttga ctgatcatat gtttagaatc ttaaatattt atgtcaagga acactgagtg |
| 142441 | ggaaacttct ggactaggtc tggaccaaag aagcatatgt ctttgattat ctttaatcta |
| 142501 | aaagatttta tgaagactaa agttttataa atagaagttt aactgatgaa taaatcagta |
| 142561 | ttacaaataa aattaacttt atttttaacc tctctgggat ctttagccag aatgagcata |
| 142621 | tataacaaaa gcagtgaaat aatatgtgtg ggtcagaacc cactgcccct cccactccac |
| 142681 | tctccttttc cctgattctc ctgtgttttt tccttcttta ccttatcttg gttccttttt |
| 142741 | tttttttttt cttttgagat ggagtctcac tctgtcgtcc aggctggagt gcagtggtgc |
| 142801 | gatctcggct cactgcaacc tccgcctcct aggttcaagc aattctctgc ctcagcttcc |
| 142861 | agagtagctg ggattacagg cgcctgctgc cacacccagc taattttttt tgtattttta |
| 142921 | gtagagacag ggtttcacca tcttggccag gctggtcttg aactcctgac ctcgtgatca |
| 142981 | cctacctcgg cccctggttc cttttttgtc tctcttgtct tccaagctat ttttttcctt |
| 143041 | ggcttttaaa ttttcttcct accctgcttt gtgtcactgt cacttaactg gcctatcaag |
| 143101 | gaaccgaact gtatttttgt tactagtatt gatttaaagt ataagtttca catttctccc |
| 143161 | aatttattat tattatttat ttatttattt gtttatttta ttttttgaga cggagtttcg |
| 143221 | ctcttgttgc ccaagctgga gtgcaatggt gtgatgtcgg ttcactgcaa cctccacctc |
| 143281 | ccgggttcaa gctattctcc ttccccactc tccctagtag ctgtgattac aggtgcctgc |
| 143341 | caccacgccc agctaatttt tgtattttta gtagagacag ggtttcgccg tgttggccaa |
| 143401 | gctggtctcg aactcctaac ctcaggtgat ccgcccgcct cggcctccca aaatgctggg |
| 143461 | attacaagcg tgagccaccg tgcccggctc catttctccc aatttcaaat tcaaggagga |
| 143521 | aaagaattcc tgattaaggt acttctttca gatcttttga gctagaacaa aaaaacaaag |
| 143581 | ggaaatattt ctaattaact ctttttaaat ttcgtttaca acgtatgata catattttac |
| 143641 | acatcctttg tggtttttgt tcgtcttgtt tttaatcaat gccttgcaag tttaccggta |
| 143701 | tttaggtagg gaaaggattt tgtttttgtt tttttaaaca aagcctatgt acattcactc |
| 143761 | agcttgggta tttgtgctat gcatgcaaat tagctataga ttagaaaacc gtattatagt |
| 143821 | ctttaaatac tggtaaactt aaattgcaga gatgcctttt aaaaatgcat agtaaaaata |
| 143881 | tttcatcttt acttttctct tcaaatgatt ttaagatttt tacatttttc cagttgatga |
| 143941 | ataacttaaa ttatgagatt tcatgggcat aattattttc tatatttatt gttacttttt |
| 144001 | aatattctta atactttgct tagaaggtat ttaaaagtga aatttcaaac tttttagtac |
| 144061 | aaaatttctt gaataaataa agttacaaaa aaaaaacaaa aacctctgag attccgtact |
| 144121 | gtatctttat gaacctccat gaacagaatt tgggatttgg gaattgcttt tccttagaca |
| 144181 | gatttagatt gttacaaatg acatttttaa gaggctgggg tggcggtagg ggttagtgct |
| 144241 | aatggtttaa cagtagggga ccatggacaa ctgtagacat cactatccag tagaacattt |
| 144301 | tgtggctggg cgcggtggct cacgcctgta gtcccagcac tttgggaggc caagacaagt |
| 144361 | ggatcacctg aggtcaggag ttcaagacca gccagaccaa catggtgaaa ccctgtctct |
| 144421 | actaaaaata caaaaaagtt agccaggcgc gcctgtagtc ctagctactc aggaggctga |
| 144481 | cacaggagaa tcgcttgaac ccgggaggca gaggttgcgg tgagctgata tcacgccact |
| 144541 | gcactccacc ctgggcaaca gagcgagact ccgtctcaaa acaacaacaa aactgcactg |
| 144601 | tccaccgtat tagctactta gctacatgtg gcttttttat tattcaaaaa taaattttta |
| 144661 | ggccgggtgc agttgctcac acctgtaatc ccaacacttt gggaggccga gatggacgga |
| 144721 | tcacttgagg ccaggagttt gagaccagcc tggccaacat ggtgaaaccc cgtctctact |
| 144781 | aaaaatacaa aaattagcca ggtaatccca gctactcaga ggctgaagca ggagtatcac |
| 144841 | tttaacccag gaggcggagg ctgcagtgag ccgagatcgc tccactgcac tccagcctgg |
| 144901 | gtgacagcaa gactgggtct caaaaataaa caaacatggc cgggcgcagt ggctcatgcc |
| 144961 | tgtaatccca gcactttggg aggccgaggc ggatggatca cttgaggcca gtagttcgag |
| 145021 | accagcctgg ccaacatggt gaaacccgtc tctactaaaa atacaaaaat cagccaggca |
| 145081 | tggtgatgct tgcctatagt tccagctact cggcaggctg aggcaggaga atcgcttgaa |
| 145141 | cccgggaggc ggaggttgca gtgagccgag atggtgcccc tgcactccag cctgggcaac |
| 145201 | agagcgagac tctgtcaaaa attaaacaaa taaatacatt tttaaaatga acgtaagatt |
| 145261 | tttacaagta caacaaactc aggttcgaaa tttacatcaa atcttttaga ccaagtcagt |
| 145321 | gcctatacaa cttggaggag ctggaagtaa acctaatgag tatgatgatg atggagggcc |
| 145381 | tgttaataag ccaccaagtt agaaaaaaag gactgtctta tagacttatg ggactgtgaa |
| 145441 | gctcaggaag gcttcatcgt ttgtacatca tttgttctag ctcccagaag acgttcacta |
| 145501 | ctcttaaaaa cattcagaga ctatgttgcc acagttttct tgttaaaata ttctggcata |
| 145561 | tgttaattcc tacagtctgg aaaattttcc cagtgtataa acaaagctgc tgtatccagt |
| 145621 | ctaaactgga tatgaaggaa tattaatgcc agctgtggca ttggcagtgg atgcacaggt |
| 145681 | gatcctagaa ctggctcttt gccttgccct ttcccctgct aagagatagc tttgcagctg |
| 145741 | gagacgtaac tgttagggct ggagagttgg tggcccttag ccctacaaca cctaggatta |
| 145801 | tagaactgct ccatgtgcct agcctaaccc tctgcacacc atttacgtgg aatataccca |
| 145861 | gagccgtcta tgctggtgac tcggcagcct tgcctaccag actgctggaa ctagggtgcc |
| 145921 | tcttcccaaa gctgtgcttg cttctctcac caatcagtcc tgcatatgtc tgtgtttgct |
| 145981 | aacacgttat atgaagaatg tggggaacta ttttggaatc atttctgtgt atgggcttat |
| 146041 | tatcttgagg gattttagga tttgtttctc aagagagggc tgggaactat accttgctag |
| 146101 | agttgtcttg agaacgctct attctcagct cattgcctcg tggaggttag ttttttatca |
| 146161 | tcggtgtgct gtccatagtc actggaagca gtgaacacat cctactctgc ttctgattct |
| 146221 | caacttactg tttttgaagc acatgaacag gccaggcacg gtggctcacg tctgtaatcc |
| 146281 | cagcactttg ggaggctgaa gtgggcggat catttgaggt caggagtttg agatcagcct |
| 146341 | ggccagcatg gcgaaacccc atctctacta aaaatacaaa aattagctgg gcgtggtggc |
| 146401 | acatgcctgt aatctcagct actcgggagg ctgaggcagg agaattgctt gaacctggga |
| 146461 | ggcagaggtt gcagtgagcc tgggcaacag agtgagtgag acttatatct caaaaaaaaa |
| 146521 | caaaaaacaa aaaactgaaa gacatgaaga aatggttttt gtaccaaggt ttggcccacg |
| 146581 | ctgagattca caaagaactg gctttcagtt cttatcttta ttttgattta aactggccca |
| 146641 | tcatgttgtc ctttgaagtt agtctagtaa atttctttcc aaagggctgg ggcactcaga |
| 146701 | agggagttta cttttctata tttatttcat aaagcaaaga tgggagatcc tccattaggg |
| 146761 | cttgggaaag taaactgagt ggcagaaggg ctcctgtgat tagctgagag agactgtggt |
| 146821 | ccttcggccc tgatgataga tccctggcct tgccacatac catacacagt gcccgcaccc |
| 146881 | ccatccccca ccacacccaa tatagtctgt gccctcagga cattgctcca gggcagtagc |
| 146941 | atggtgaggt tagcctgatg atggccttga gctaaagagt gtgcacctaa aatgcacttg |
| 147001 | tttgagtagt ttctgcctat gccttcaagt tgcctttttg ggaaaaccta gtgaccgtta |
| 147061 | agagtaaatg caaactaatt tgattttaat atcatatgta gagctgtatt atatgaacca |
| 147121 | aatgctagtc tgttaagcaa tagctacact tattttttca agacaatgga tggtttaaat |
| 147181 | ggagtcatct atagaaattg gtagtggcgt gagttatgca ttgtaaccat caagaaagtt |
| 147241 | cagttgatga agtgtagagg agcgatggag gttgtcagac atcggttgtg tacatgctcc |
| 147301 | tttttctttc actttagttt ccacgggctc ccttgctcag cagtatgcgc accctaacgc |
| 147361 | taccctgcac ccacatactc cacaccctca gccttcagct acccccactg gacagcagca |
| 147421 | aagccaacat ggtggaagtc atcctgcacc cagtcctgtt caggtaaggg caactcagag |
| 147481 | gtctgcatgg agtggcttct ttatcctagt atctgagtgc tttcttcagg tgccaggtat |
| 147541 | cgcatcgtca gaacacatgg catgtccacc ctcgtgaaga tggatacagc tgtgcccctg |
| 147601 | gggtggtggt tttaagaatc acatttaaag gctgggcgca gtggctcacg cctgtaatcc |
| 147661 | caccactttg ggaggccgag gcgggtggat cacgaggtca ggagattgag accatcctgg |
| 147721 | cgaacactgt gaaactccgt ctctaataaa aatacaaaaa aattagccgg gcgtggtggt |
| 147781 | gggcgcctgt agtcccagct tctcgggagg ctgaggaagg agaatggcgt gaacccggga |
| 147841 | ggcggagctt gcagtgagca gagatcgcgc cactgcactc cagcttggac aacagcgaga |
| 147901 | ctctgtctca aaaaaataaa aaattaaaaa aaatcacatt taagatacat gttgataata |
| 147961 | aggtgattgg ataagctctg gaaacttgca gtaatgaaaa atcaaattta acataaagtt |
| 148021 | cataaggcaa attcctattt gcttgggact ttttaatttc taaggtttat gtgatgaggt |
| 148081 | tattttccta tgagcttctt gaattatgtt tgctaatgga ggcagttaaa gatgtctttg |
| 148141 | atatctatca gttccctggg gcagtagtct tttttgactt tagtatgtat gctcagaagt |
| 148201 | ttctaactgc cagactgaga atcaggcttc tgtaccctag aaaggagttg tccagatggg |
| 148261 | aggcacctcc agccttgctc ttaccaccct gtacattctc ctgtactttc cagtgaccct |
| 148321 | catcataggc ccaagtgtgc aaagcttagc tttgtgggta tcccttggct gcttttcatt |
| 148381 | aaagaagttt tcctctcaat tctttcctgt cgctttgcag caccatcagc accaggccgc |
| 148441 | ccaggctctc catctggcca gtccacagca gcagtcagcc atttaccacg cggggcttgc |
| 148501 | gccaactcca ccctccatga cacctgcctc caacacgcag tcgccacaga atagtttccc |
| 148561 | agcagcacaa cagactgtct ttacgatcca tccttctcac gttcagccgg cgtataccaa |
| 148621 | cccaccccac atggcccacg tacctcaggt aataccagct ttagccaact ttctgtgaag |
| 148681 | gccaagtaga atgtgaaggt tatcagtaag cagctagagg ctctcccagc taggaaaccc |
| 148741 | tgtgtgtcat gccatttgcc tgtctccctt tccctctcaa atacacgtga tctggcccta |
| 148801 | agggaatgtt tgtgtggttt tgtcatggga tcagtgaagg tgctgattgg tcagtccttt |
| 148861 | agttttccaa ctgagacctt aaaaatatct ttgactctgg aatgcaaccc agtccttctt |
| 148921 | tcctttctgt gtctgctttg ctatgtctat atagcctcac tactatatat atgtgtacat |
| 148981 | atatattccc ctacacactt accttggaag ccaggcaggg atgatggcct tcacagagtc |
| 149041 | tcagctctcc gaagtgacta ccggggcctg tcaacttgat tgttactcac atgagttcca |
| 149101 | gacacatctc tccaattgtt ttccctggtt atccatatat ctgctttgac cataagttgt |
| 149161 | actcttgaga gggcttggcc ttggacattg gtgcagtgta actagaagct ggaagcaccc |
| 149221 | aggtggtccc atttttcttt aagagcagcc ctggaagcac tttggagctc acctccagtg |
| 149281 | taagctgcta caggtgaaag gtgtgcttgc catctcagtg gttgctgtct gcatcagctg |
| 149341 | ctgacaaagg tccctgcact ccagggccca ggggattgtc ttaatgagga gaaggagctg |
| 149401 | cactgaagtt gggctctaac gctggccttg aggccctccc tggggctgct acgggtgaat |
| 149461 | tggctgtatt agatgtctct gctactttca taacagaact ctctgaggcg ggtctaagtg |
| 149521 | agacctgcca caatgaattc catttcctgt taaatagtgc gccagtgagg ctctggcaag |
| 149581 | gtgtgggcta gagatgcgac tcagttggat ctatctctca gaaggctacc ttgtaagtag |
| 149641 | agttccacag ctctgggaag tttgggcgtc ctcaccctgc aaagtttagg ttctgtggtg |
| 149701 | tagcgcactg cagttgattt gctttttgat agtggggagg gaagccggct tggtccgtgt |
| 149761 | gggccagcgt ggtttggtgg agtcagcttc ataagagctg gggtcctgta ggtgtctacc |
| 149821 | agaggctggt ggctaagtag gcatgtgaac ttacatgtaa gtcagggatc cctaaaacct |
| 149881 | cactctgttt ttgtgctgaa agggcaaaaa ggttaacaca gggaagctca aatttgccat |
| 149941 | gtgcccgttt gaatatgtga gagtaaaaac ggcatttcat ccaaggctta tcgtagtcta |
| 150001 | gaacagtgca cagtgtggga aaaaggaaac aagggctctt cctggccctg ccaaccccct |
| 150061 | gcagagctgg aatccagctg tttgggctga ctaaaatcac ctttccaact tgacagtgag |
| 150121 | tgagaccagg ttgaacttgg tacagagacg ctgggctggc ccagatgact tcaggttact |
| 150181 | cctttccatc tcactggagc cattaaaaac tccaactcct cctcctcctc ctgctccatc |
| 150241 | agcatatctc tgagagagtc acgggggcct aagagtctct tttcactgcc tggtgagcag |
| 150301 | accagaagca gagggagaga ggcaaatgaa cagaggtcca agtaattcac atacttgact |
| 150361 | gtgacagtct ctgcttatta atgtaatctg ttttcctatt tgaaagggat gttatctgca |
| 150421 | aaactacctc aggccccaca tggcagcctg attctgaagc atcattgaat cttgtatgat |
| 150481 | attaagttga gaaagctgcc cttggatcca gtgtctaatc tttgtgaaga tcttacccca |
| 150541 | tacatagaat acaatgatca gaaatgtcaa gggttaggac agcacagccc tgacttctac |
| 150601 | ccaggctcac ttgttgcctg ctccctgacc cttgcaggat ctgcccaaag gtgaagcgcg |
| 150661 | tcttcaggtc aatagataat ctactagaga ttgtccccag agaacagaac tgggccctga |
| 150721 | ggcccaccgt tgccctttcc tgagagtccc agcccagtga aaggaacaca gttgacatgt |
| 150781 | tgttgaagcc ggagatgttg cctgtatgcg taaaagagct ctctgtttca ggctcatgta |
| 150841 | cagtcaggaa tggttccttc tcatccaact gcccatgcgc caatgatgct aatgacgaca |
| 150901 | cagccacccg gcggtcccca ggccgccctc gctcaaagtg cactacagcc cattccagtc |
| 150961 | tcgacaacag cgcatttccc ctatatgacg cacccttcag gtgaggcgtg tgtgtgcagg |
| 151021 | ggccgccggg gcaccccaaa gcattctgct cgcacaggtg gaatggcagg cagggccagt |
| 151081 | gcttcaagcc ccgcatttga gaactagcaa gacccgtcca ggagtgtgca caggagggac |
| 151141 | tgtgacgatc agttcagcat cagggcctga ggcttccggg agccgagtct gtgtgtgttc |
| 151201 | tgatggtata caggatttgg cttgatgaga agcagcagca gcagcaacag cagcctgatg |
| 151261 | catgcctagg actcagttgg ccttccttgt tatgacaggc tggacagggc agtgttttcc |
| 151321 | ttcctgagtc ccaaaagtct gacatgtggg gggttattac catggcagag tttgattgta |
| 151381 | gctctggaga agatactgct gagaaagcgc tgtggatgga ctggctttga gtgtagcgtt |
| 151441 | agccccagcc cctgaacagg ggagagcgcc ctgtgattgt gctctactac ttgatggctg |
| 151501 | ccatggcgat acttcacagt ctgacctgtt attctgaaag caatactggt gcttggctaa |
| 151561 | tatttgggga gggggtttgt taaggccttt ttttctaccc catgaacaag tcttctggga |
| 151621 | gttttatctg aagtggtttt acgtctgact ggcttgtttc tacccaccca cccaaccctc |
| 151681 | cccactttgg tgcagatggg agggggaaaa gcgaattcaa ttttgagttt tgttcagcta |
| 151741 | gcacgaggat agtttacaat catgtgctgc agagacacta ggctgatgtg tggtgttgcc |
| 151801 | agttttctgt ttcaatgttc gcttttcttt ttacagtaca agcccaccac caacagcagt |
| 151861 | tgtaaggctg ccctggagga accgaaaggc caaattccct cctcccttct actgcttcta |
| 151921 | ccaactggaa gcacagaaaa ctagaatttc atttattttg tttttaaaat atatatgttg |
| 151981 | atttcttgta acatccaata ggaatgctaa cagttcactt gcagtggaag atacttggac |
| 152041 | cgagtagagg catttaggaa cttgggggct attccataat tccatatgct gtttcagagt |
| 152101 | cccgcaggta ccccagctct gcttgccgaa actggaagtt atttattttt taataaccct |
| 152161 | tgaaagtcat gaacacatca gctagcaaaa gaagtaacaa gagtgattct tgctgctatt |
| 152221 | actgctaaaa aaaaaaaaaa aaaaaaatca agacttggaa cgccctttta ctaaacttga |
| 152281 | caaagtttca gtaaattctt accgtcaaac tgacggatta ttatttataa atcaagtttg |
| 152341 | atgaggtgat cactgtctac agtggttcaa cttttaagtt aagggaaaaa cttttacttt |
| 152401 | gtagataata taaaataaaa acttaaaaaa aatttaaaaa ataaaaaaag ttttaaaaac |
| 152461 | tgatcaagtt agtgtgtgtc tgtataagct acttctttgt aggatactta atatcaaagc |
| 152521 | aggtgtgcta agggtgcatt ttgaatatcc cggaaggtag ctgtgaaatg atttttcttc |
| 152581 | ttcaccctta gttctggttc aaggtatctc tagaaaaaga caagactgag ctattctctt |
| 152641 | tggtggatta gagatctgct tcaggaggag gaaggttggc cagagttggg cagcactgaa |
| 152701 | attccacatc ctcgggctga caccgattct gtaagcttcc tttttaatat ctcctgaacc |
| 152761 | aaaatgagtg tcattagctg gaagttccca attcgggcat ttttctactt taccagtagg |
| 152821 | gggcaggaga cactcagaaa aaaattgcaa taaagaaatc cagagggcat gaaggctgaa |
| 152881 | aagatacaaa gatgtacaaa gctgcttatt gacatggatg gactcataag catttgttag |
| 152941 | tattcccaga ttgcaacggg gaggacaaag ggaagagcga gtatttgggc agggcaagga |
| 153001 | ttttgtagag acaccatggt cttaatagag cctttaaata ttatgacagc aaatcaagat |
| 153061 | tctgaaaact ttttaattca catatagcaa ttcgtacatt atagcaaaat ttgcattatt |
| 153121 | caagaataag ttacttgtac agtacataaa acaatacata aaaatttgcc aaataccttc |
| 153181 | tgcctataat gatacaagat gaatccactt tatgttatca caatgtgctg tatattctaa |
| 153241 | ccaaacacag gatgtcagat gtgtccttgt taatatactc gcaagttcct ctagcttgtg |
| 153301 | ggagatgtta gagctaacac atttgcagta agggacttag tcctgaatag aaagcatgaa |
| 153361 | ggaatctcag gcaaccctca gggaagagtc caaggccttg actttaggct aagaaactgt |
| 153421 | tatgtaaaaa tagtgttctc tggcccaaga ttttaatgat tgctattcct ttttcctacg |
| 153481 | gtccagaaat gatcaaaggc agaagattta taccagataa agccatatgg attgctggtc |
| 153541 | taaaattcaa ggcaggttag ttgacttaat tctttggtgc tggtgactgt tagtttgtaa |
| 153601 | aagttcaata agtcagatga aggaagggat ggtgccggga gctgtcaagc tgtactggtg |
| 153661 | gggtctgtaa ttagagctaa ctggagggat catgatgtct actgtccagt ttggtgttga |
| 153721 | gccatggctc tcggtagaag ttgccggctg gggcctggtc aggactggaa ggagagtggt |
| 153781 | gggatgtgct gtgcctatgg tgggctagct gcagccagtg gggtgcctgc cccacactgc |
| 153841 | tgcccacccc ttcatcagct gattctgctc ccacataaag aaaggtgtcg gcttagtgtc |
| 153901 | acttcttcct agagccatgg gagttttctg tcagcatgtt tttgagctgt cctggtaact |
| 153961 | tggacgggaa gcagtctgga ggtgggtgcc ttccaaatct ctgccacaga atctgaactt |
| 154021 | gcactttgca gcctgttagt cagagctgct aagtttgctc tacctggtgt gcctcttggc |
| 154081 | accacctgag agatgtatgt agagaggctg ctaagaaatg agtgcccctg gcatgaagac |
| 154141 | gtcatatccc agatgtttaa cttctggctg gagggaggga acttaatttg gttcattatc |
| 154201 | tactgggtac ctttcctgtg cggaggggag gaccagtgac tttccataat aagctttctc |
| 154261 | ctttaaaaaa acttcagact cttacttact ctcttggttc acactaatta gcattcattt |
| 154321 | tccagagaaa gctgcttgac tttgaggaaa taaagacagg tgtccttaaa aatggactgc |
| 154381 | aaatctggat cagtgtcagg tctgctttag ggtccatgaa atcacccaag agtgttggta |
| 154441 | ctaaaagtgg tgtaggctgg gcgcggtggc tcgcgcctgt aatcccagca ctttgggagg |
| 154501 | ccaaggcggg tggatcacct gaggtcagac attcgagacc agcctggcca acatggtgaa |
| 154561 | accctgtctc tactaaaaat acaaaattag ccaggcatgg tggctggtgc ttgtaatccc |
| 154621 | agctacttga gaggtcgagg cagaatcgct tgaaccagga aggtggaggt tgcagtgagc |
| 154681 | caagattgca ccactgcact ccagcccggt tgacaagaga aactccatct caaaaaaaaa |
| 154741 | aaaaagaaaa gtgcataggg gtttgttccc ccaccctctg aaagacccca aggaaattct |
| 154801 | agaagcaaaa atttgacctc ttggagcaca gagactaagt ataagtggaa gaagcagacc |
| 154861 | taaccctcct cccaggcagt cagtcctatg agctttaggc tgaggtttca ttttagcctc |
| 154921 | tgatcgatgt atctgtaaag ctgatctaaa ctcccagttt cctagaagcc actgctgtga |
| 154981 | gcctgggggt tgtactgaat ggctctgctg gcacccaaga gcagctgact ccactgtcac |
| 155041 | tgcccccgtt tctgagcctc cagacaggtt ctgggaggag ccagccccct atgaattgta |
| 155101 | tcacttgtat agatagagat ttgacttcag gacctccctg gatggtggag attgtgtgtg |
| 155161 | cccatgtgtg caaggagata gatagattct caatccagtg aagcagtagc cagctagaac |
| 155221 | agaacactga ttgtgggcta ttaaaggagt ttctcattag ttatggcacc agattcccag |
| 155281 | cttccatttt ggctcaaaaa aggagcctaa aacccacttg tgggataatt taataagcca |
| 155341 | ttagatgctt ggagggcacc aagccagcct tgctgctgaa ggatttacta gaaagggcct |
| 155401 | aggagtcctt cttccctttt tgcttgctct tcccctagcc ccagttaatt taactatcac |
| 155461 | tgtaaagctg ctgtaaacca gaatccagtt ttttattcgt aaggcaccag gatagaactg |
| 155521 | ttcttgccac cctcctattt cagtaattgc ctgaatttcc ctgaaagggt gacatacttc |
| 155581 | acttcttgac atttaggaag gacagtggcc actcagtggc aggtgactgt cccgaggatg |
| 155641 | cagtagacaa gccagcatgc tggctcagac tgtaaattag gtagggtttg catagctgtt |
| 155701 | tctgactaca aaaatagcat gggtgttagt tgaaacagtg cactgaaata agcagtggtt |
| 155761 | aagttttgaa cctgaatcct ttaaagaaat tagtctgagg taaaatgttt taaatcattt |
| 155821 | ttgaccttca gcttgtgcct ctgacagagc ttctgtccta aagctgagag tagaagcagg |
| 155881 | attagatgtg gtcatctgtg gtgtggacac agcagacctg ccatgtggac tgacggtaga |
| 155941 | tgggagaacc gcttgttagt gttctgggct tcctgatgat ccccccctcc tccccgtaga |
| 156001 | aagtctgtca tccgctgtga cagtgtcagt gaaaaagaga acaagtttcc actgccagct |
| 156061 | ctggccctca ccccaactcc tttctctaag agtaacgggg actgggcata gggaccccgg |
| 156121 | gcaggagcta gcggcatcct ctaacaagaa ctacatctgt aaacaataag gagaaagcca |
| 156181 | ggtggataga tgaaacctac aggagggccc ttcaacaggc ctaatagaag ggtcacttcc |
| 156241 | aaactggcac gagcagttaa cagtgtccct taaatctttc tctggaagga aggaaagatc |
| 156301 | aattacattc acaagttcac acttctgcct gtgctcctca agggcagctg ttgaggcctg |
| 156361 | gaattcctca ctggtcagag aggtgtgtac tgattgtcta tggcccgcag gtggctccgg |
| 156421 | gaggatgagt ccatttcgta gtccgagtcc cgggctcgat tcacaggctc atgctccagg |
| 156481 | tgctgcaggc tgttggccag ttcttcgggc gaaggcacca ggtggaagat ctgctcgggg |
| 156541 | ggtgaggatc gccctgggag accctctggg ctgagccccc ggggacagcc agaagaacta |
| 156601 | aggtggggaa ggcccaactc tgaaccccag tgaggaaggg actctgaatc ccagtgagga |
| 156661 | agggagaaag ggaagaggac cgtgttgcag gaacctagga caagtgacca agagagacca |
| 156721 | caccatcagt cttgaggcaa gtgctgacag gacacagaac aagatacctg tacccctggg |
| 156781 | tctgcagcaa gcctctaccc tctacccagt gtcagccacc cccttcagca aagggcagta |
| 156841 | gggtcagacc tggtggttgg gagacgacta ccacgtagct ggagagccgg acatcacagg |
| 156901 | cggcgccgca ctcgagtggg atgggcgagc gctggaagtg gtggagcatg tccacgaccg |
| 156961 | agggaaagtg gaggtgctgc acacggcact ggccccgctc tgtcagcgac aggcgcaggt |
| 157021 | gctgtgggag gagaggatgg tgggctcggg ctgtccctct aggaccctga actccagtgg |
| 157081 | agaggggtgg cacggtcctg cctgccccac cccaccccac cccaccccat accttggcta |
| 157141 | tcccctgaaa gttgaaagtg agcacgtatt ccccacgccg cgtctcgctc tgccgcacca |
| 157201 | ggaacactcc atgagcatca gggccctgca gctgaaccag ctgagctgct ttcactctgg |
| 157261 | agatggggcc gtggaaccag gggtagcagg acaggaaatg gtccgtcttc tggcaggccg |
| 157321 | ggtccagcag ccccccagga gaagcacctg ttagataaga tgggtgatgg tggtcgcagg |
| 157381 | gggctacctt atgtcctggg ctgagtctgg gttccagcgt acccagaaga caatggccta |
| 157441 | gctattggtt tacccacctt ggttaaggga atctgtgctg ccccttgggg agctggacgt |
| 157501 | gctaggctct agggctgagg gaatatgcat ctctgcttct gtgctctcca gcctgctgga |
| 157561 | gaagaggctg aggttaagga tctgggcagg tcaggggtgg tagcagcaaa ggtggtgcca |
| 157621 | gggacgaggg cccaggacct caccctcggc ctgtgcactc cgagagctca gccatccatg |
| 157681 | aattcagctg ctgctcgtct cccacctcaa agatgatgtc tgtccggtcc ttcacctggc |
| 157741 | agggaggaag ggaacagggt tgggtctgct gtatgtgtat gtatttgtat tgccccaggg |
| 157801 | tgtgaaaagc cttgtcactc accttcagca caaaggtgta aaggttgtca ggcatctcaa |
| 157861 | gccgtgtgca ccaccggacc tcctggatgc tggagcaagc tgcttgtagc ttgggccttg |
| 157921 | aactctacga gaccaagttg gggtactcaa ggcttgttcc tgatgctctt tcttccagcc |
| 157981 | aggcctgagt ttgtctatag tctccccagg agtccccctt ttagagagag tatccatggt |
| 158041 | gttgctgggc aggctctgag cctgtagtga ggtccagggt gccttcccct agcctcctga |
| 158101 | acaccattca cactgagatc ccaagaagca tgtgctggca gaaggccagc cctcccagcc |
| 158161 | cattggacct ggacttggag acattcccac tctgccatgt gtctgacatc cgtttctctt |
| 158221 | cccactggtg agcaaacagc tccctgccca gccaggtgat agcagctgct gacggtctca |
| 158281 | tcacaccaag cccaaagggg cagagcctag catggctagc ctaagttata gccactgtaa |
| 158341 | aaccacaaaa tcttggccct ggaccttccc aggacacccg gcacataggg ctgcagggtg |
| 158401 | gataggagtt agccactgtt cctttgccag caggaaacct gctcccaggt ctcaactgac |
| 158461 | cctgcagatg cttcctgtgc tggggcactc tgcagccaag gagccagctg cttccggttg |
| 158521 | ggaatctgct caggaggggg tttgcccctc cccttcctct tgtgataaga ctgaaaagat |
| 158581 | ggccgaaaga agaaaagggg agaaagagat gagataagga atggacagaa gtggccccac |
| 158641 | tgggccccct ccattcaggt gttcctggaa aatactacaa cccctaacaa actgggagca |
| 158701 | gggggatgga aagcagaggc agcccaaagg atgttctagc aactgtggct tcaggtgatc |
| 158761 | tgacctccac ctcagcccaa aagggcatac cttggactgg agctgctagt gctgttgaat |
| 158821 | tgacccccaa aaccagatgc ttaaccttgc tttacacaca gagctgccag gtacctctgt |
| 158881 | cggaaggatc atgacccagt ggcctggcca ccccctttat cccccttccc cacctgcagg |
| 158941 | agagtcaatg aacagtgagg taagagggca gcctggcagg agcaggatgg ccacctgggc |
| 159001 | ccttccagca ggtctcctgg gagctcccgg gcctgctctg gttggcggac acaacaggct |
| 159061 | gggggagagt aatgactgca ggaatgacca acactggcca cagctcactg cggcctctca |
| 159121 | ctagaaagct gcatgactag aaaattccag cctgaagtga ccagcaccag ccagggcatt |
| 159181 | ttcacctcct actcccagag gtccagcagc caggatgagg caggcagagt gaggcctcac |
| 159241 | atggcatcca cacgggcact ggacacagcg cagtgtgcag ggagtggggc ctctgcgctc |
| 159301 | ttgcccattt ttacagccag ccagccctgc agagccttca tttcctgtct gcaagatggg |
| 159361 | taaggtgcga gtggccactt ccttctgcta ctcttccagt tggccctgcc tggcccccac |
| 159421 | ccttccccct agctgcaggc cttaaggatc actgggagga tataggccag gcctggaggg |
| 159481 | gaacgggcct agtctccctt tcagttccgc tccaggccta gtccccagtg tcagtgctga |
| 159541 | gattaaccag cagagctgcc ggtttcactc tgactctggc ccctgcaagc cagctccacc |
| 159601 | cccagcagct gaagaactgc tgagcaggca ggcagtggtg aaaagaggat tcccctcctc |
| 159661 | acccacagcc agcctggctg ccacctgcct gctggctgag cacaacccag gggttcttcc |
| 159721 | ccaactcccc acagcagccc ttgagagccg cccaggccta gagccacaat ctcaacccaa |
| 159781 | gcctgccaag tatcagagcc ggggtccgcc acagctacct ggtggtatgg gtgtcctatg |
| 159841 | ccagaggctc cagccaggtt gcagcgtgtg ggcacatggg cgcaggacaa cccctgcttc |
| 159901 | agtcgaaacc tcagcctgga cagctcaggc tctggccact gcagggaagg accaccagat |
| 159961 | gcccatcaga ttaagctata tggggggatg ggtgagaggg gagacaagct caaacctggg |
| 160021 | ggctgtgtca gcagctgtgg gggctttagg ttccctcctc tcacatgccc gctattccac |
| 160081 | ctccctcaaa agtcaagcca ggccagcaac agctgctccc tggacctgct taggctctac |
| 160141 | tgtggttggg tgatcaaagt ggccagatca tttcaggtat ttgtggttcg aacagtagac |
| 160201 | tgtcacccac cacatcctgc agcgtctatt tctggggagc aagtcttctc cattcccagc |
| 160261 | atctaaacag atggcttctg tttgttatgg ccactgtggc tccagtggga ataatctatt |
| 160321 | tctttctttg ctctggctgc taggaatctc aaagtcaaag acgtaggcca gctctgacac |
| 160381 | taccccaacg gtaggacttg tatttatttc aacagctaac tttatcccag atgttatagt |
| 160441 | tcttttatcc tctagggttt tttgtttttg tttttgtttt ttgagacaga ttctcactct |
| 160501 | gttgcccatg ctggagtgca gtggcgcaat ctcggctcac tgcaacctcc acctcctgag |
| 160561 | ttcaagcgat tctcctgcct cagcttccgc agtagctggg attacaggca cctgccacca |
| 160621 | cacccagcta atttttgtat ttttagtaga gctggggttt caccatgttg gttggccagg |
| 160681 | ttggtctcga acttctgaca tcaggtgatc cgcctgcctt ggcctcccaa agtgctggga |
| 160741 | ttttaggcat gagccaccac gccgggccct accctctagg ttttaagatt cagccctgac |
| 160801 | cctgccactt cctatacaac cctggacaaa gggttcggct ctccatgcct tagttgtcat |
| 160861 | ctgtaattca gctcattttt tcacaaggtg agctaacaca agcgaaaggc atgactcgca |
| 160921 | cagctgttaa caggccggtg agtaaagcct gcttggctcc cactacacta ctgacagcat |
| 160981 | gacagtcccc tctgagtagc ctagccactg ggtacacccc cagcttctct cagccttaca |
| 161041 | gaactattgc cccattttgt cacatgctcc cttaccatcc tgcaataaat tccacgtaat |
| 161101 | ataaccaaca tactacccta actaattaca agacagggac aacaaaggac tgtcacttag |
| 161161 | ccgaaataaa acgtgcttca gtgcaactgt agacaacaca accaactact tggctgcttc |
| 161221 | tgagtttgtt gtgaaaagaa ggctaagaac ataagggagc caaagggcgg gggagctagg |
| 161281 | aaagccagcc tggttccaga tccacccagg gtccacatgg aaatgcagct ccaggctccc |
| 161341 | ttcaaatggc cctgtgctgt gatcagtccc cctgcaaagg tctctgagcc acagtcccgt |
| 161401 | catctgagaa gtgaagatga aacaccagcc tcgtggggtc acgcagctct ccctgactcc |
| 161461 | cgcctacctg aggatccctc tttatcccac ctggctgttc agcccgcccg gagggaatgt |
| 161521 | gacgggctgg aggcaacaga cccttcctct ctaggcctca cttgccctca tgctgttacc |
| 161581 | tggaaactgt cttgctacct actgcccctt ctgtgtgctg agggacaggg gacctgtctt |
| 161641 | ggcacaaaca tttatagggc caggatggtc cttctagagt cttccgaggt actgggccac |
| 161701 | accccaccct gccaggtcct cataaccagc ttgagtgggg tctgttactg gctttgccct |
| 161761 | caggcatcca gcagagacag gcaagtcggg gcagggcagg gctggacaca ccagtcaaag |
| 161821 | gccagagctc tggtggcgtg accacaccta cgtggagagc aaggctgatg gcatggcctc |
| 161881 | ccctggtgag agagcaggaa gaggcacagg tgagcctgcc acagcagccc ccagaagcca |
| 161941 | ccatggggct tcaaggctgt gggaatcagc ttgagctttt tctaaggtgg ggaagctctt |
| 162001 | aacaggagca ggcctgctgg ctgtccccct cctcctggga cgctggaggg caggacacag |
| 162061 | ggcagcatct gacacacccc agcagcaagg gcttggccca gaccagccct ggggagagac |
| 162121 | cgcccacccc cgggaacccc caggccacac tagctccctg gtggtcttac cttccccaga |
| 162181 | gcctttgagc ccacattcat cggtgtacag cagcagtggg acaggcctca gtcgaccctc |
| 162241 | cagcaccagc cacgcctctg ccacacctat gccctcaagg aggcatctgg cagcctgggc |
| 162301 | ctagctctgc aggtggttag aagagcccta gctctaccac ttacttacct ttgaaacctt |
| 162361 | gggcaggaca ctaactggag gttcagtttg ctctcagtaa tagggggccc gtaagcaggt |
| 162421 | cctgttgacc ttcttactgg aggaggagag ggagggggtc tgaaaaggcg cagagcaaat |
| 162481 | tccagatcct gtcctggatg gctgatccct caccccagcc cagaacctga agtggatgct |
| 162541 | gccgggatgg atgaatgagc attattatgg aaaatactta atccaagctt actgtctagt |
| 162601 | atcaaaaagt taacctactt gggaagctga ggcaggagaa tcacttgaac ctgggagaca |
| 162661 | gaggttgcag tgagctgaga tcacaccact gtactctaga ctggtcgaca gagtgagacc |
| 162721 | ctgtcttaaa aaaaaaaaaa ttccttgcct tcttccaaaa atgattttag tcaggttaga |
| 162781 | gacacccaga aagagctggg agacagctgc tacaacacag acaagagtcc ctcttgccac |
| 162841 | agatttgtct cagcttcctg gcagccaatg caaaggattc tccgagagag gacagtcatt |
| 162901 | tgtggaaaga gaatgctgct gcttagctcc attaaagact ctgagaagtc ctgcagcaga |
| 162961 | gaaacctatt aaacccatgc attacccaga ctttgaccta tttcatagac cttattacca |
| 163021 | tcccaaggta atatgggatt gaggtgagga aggtcctcac ctcgcaaact ctccttaaac |
| 163081 | gtggtgcgtc tcagccattc tctggggtgg gcagcatcaa gactgagcct agttgctaaa |
| 163141 | agccatccag tatcctgccc agaaatccct cagggctaca gaaccatgca gc |

In various embodiments, the ATXN2 gene product is an ATXN2 mRNA isoform, comprising NCBI Reference Sequence NM_001310121.1 (SEQ ID NO: 149357). In various embodiments, the ATXN2 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with an ATXN2 mRNA isoform, comprising NCBI Reference Sequence NM_001310121.1 (SEQ ID NO: 149357).

| NCBI Reference NM_001310121.1 | |
|---|---|
| (SEQ ID NO: 149357) | |
| 1 | cccgagaaag caacccagcg cgccgcccgc tcctcacgtg tccctcccgg ccccggggcc |
| 61 | acctcacgtt ctgcttccgt ctgacccctc cgacttccga ggtcgaaaca gtaacaaagg |
| 121 | actgcctcag tctacgattt cttttgatgg aatctatgca aatatgagga tggttcatat |
| 181 | acttacatca gttgttggct ccaaatgtga agtacaagtg aaaaatggag gtatatatga |
| 241 | aggagttttt aaaacttaca gtccgaagty tgatttggta cttgatgccg cacatgagaa |
| 301 | aagtacagaa tccagttcgg ggccgaaacg tgaagaaata atggagagta ttttgttcaa |
| 361 | atgttcagac tttgttgtgg tacagtttaa agatatggac tccagttatg caaaaagaga |
| 421 | tgcttttact gactctgcta tcagtgctaa agtgaatggc gaacacaaag agaaggacct |
| 481 | ggagccctgg gatgcaggtg aactcacagc caatgaggaa cttgaggcut tggaaaatga |
| 541 | cgtatctaat ggatgggatc ccaatgatat gtttcgatat aatgaagaaa attatggtgt |
| 601 | agtgtctacg tatgatagca gtttatcttc gtatacagtg cccttagaaa gagataactc |
| 661 | agaagaattt ttaaaacggg aagcaagggc aaaccagtta gcagaagaaa ttgagtcaag |
| 721 | tgcccagtac aaagctcgag tggccctgga aaatgatgat aggagtgagg aagaaaaata |
| 781 | cacagcagtt cagagaaatt ccagtgaacg tgaggggcac agcataaaca ctagggaaaa |
| 841 | taaatatatt cctcctggac aaagaaatag agaagtcata tcctggggaa gtgggagaca |
| 901 | gaattcaccg cgtatgggcc agcctggatc gggctccatg ccatcaagat ccacttctca |
| 961 | cacttcagat ttcaacccga attctggttc agaccaaaga gtagttaatg gaggtgttcc |
| 1021 | ctggccatcg ccttgcccat ctccttcctc tcgcccacct tctcgctacc agtcaggtcc |
| 1081 | caactctctt ccacctcggg cagccacccc tacacggccg ccctccaggc ccccctcgcg |
| 1141 | gccatccaga cccccgtctc acccctctgc tcatggttct ccagctcctg tctctactat |
| 1201 | gcctaaacgc atgtcttcag aagggcctcc aaggatgtcc ccaaaggccc agcgacatcc |
| 1261 | tcgaaatcac agagtttctg ctgggagggg ttccatatcc agtggcctag aatttgtatc |
| 1321 | ccacaaccca cccagtgaag cagctactcc tccagtagca aggaccagtc cctcgggggg |
| 1381 | aacgtggtca tcagtggtca gtggggttcc aagattatcc cctaaaactc atagacccag |
| 1441 | gtctcccaga cagaacagta ttggaaatac ccccagtggg ccagttcttg cttctcccca |
| 1501 | agctggtatt attccaactg aagctgttgc catgcctatt ccagctgcat ctcctacgcc |
| 1561 | tgctagtcct gcatcgaaca gagctgttac cccttctagt gaggctaaag attccaggct |
| 1621 | tcaagatcag aggcagaact ctcctgcagg gaataaagaa aatattaaac ccaatgaaac |
| 1681 | atcacctagc ttctcaaaag ctgaaaacaa aggtatatca ccagttgttt ctgaacatag |
| 1741 | aaaacagatt gatgatttaa agaaatttaa gaatgatttt aggttacagc caagttctac |
| 1801 | ttctgaatct atggatcaac tactaaacaa aaatagagag ggagaaaaat caagagattt |
| 1861 | gatcaaagac aaaattgaac caagtgctaa ggattctttc attgaaaata gcagcagcaa |
| 1921 | ctgtaccagt ggcagcagca agccgaatag ccccagcatt tccccttcaa tacttagtaa |
| 1981 | cacggagcac aagaggggac ctgaggtcac ttcccaaggg gttcagactt ccagcccagc |
| 2041 | atgtaaacaa gagaaagacg ataaggaaga gaagaaagac gcagctgagc aagttaggaa |
| 2101 | atcaacattg aatcccaatg caaaggagtt caacccacgt tccttctctc agccaaagcc |
| 2161 | ttctactacc ccaacttcac ctcggcctca agcacaacct agcccatcta tggtgggtca |
| 2221 | tcaacagcca actccagttt atactcagcc tgcttgtttt gcaccaaata tgatgtatcc |
| 2281 | agtcccagtg agcccaggcg tgcaaccttt atacccaata cctatgacgc ccatgccagt |
| 2341 | gaatcaagcc aagacatata gagcagtacc aaatatgccc caacagcggc aagaccagca |
| 2401 | tcatcagagt gccatgatgc acccagcgtc agcagcgggc ccaccgattg cagccacccc |
| 2461 | accagcttac tccacgcaat atgttgccta cagtcctcag cagttcccaa atcagcccct |
| 2521 | tgttcagcat gtgccacatt atcagtctca gcatcctcat gtctatagtc ctgtaataca |
| 2581 | gggtaatgct agaatgatgg caccaccaac acacgcccag cctggtttag tatcttcttc |
| 2641 | agcaactcag tacggggctc atgagcagac gcatgcgatg tatgcatgtc ccaaattacc |
| 2701 | atacaacaag gagacaagcc cttctttcta ctctgccatt tccacgggct cccttgctca |
| 2761 | gcagtatgcg caccctaacg ctaccctgca cccacatact ccacaccctc agccttcagc |
| 2821 | tacccccact ggacagcagc aaagccaaca tggtggaagt catcctgcac ccagtcctgt |
| 2881 | tcagcaccat cagcaccagg ccgcccaggc tctccatctg gccagtccac agcagcagtc |
| 2941 | agccatttac cacgcggggc ttgcgccaac tccaccctcc atgacacctg cctccaacac |
| 3001 | gcagtcgcca cagaatagtt tcccagcagc acaacagact gtctttacga tccatccttc |
| 3061 | tcacgttcag ccggcgtata ccaacccacc ccacatggcc cacgtacctc agtgcgccag |
| 3121 | tgaggctctg gcaaggtgtg ggctagagat gcgactcagt tggatctatc tctcagaagg |
| 3181 | ctaccttgct catgtacagt caggaatggt tccttctcat ccaactgccc atgcgccaat |
| 3241 | gatgctaatg acgacacagc cacccggcgg tccccaggcc gccctcgctc aaagtgcact |
| 3301 | acagcccatt ccagtctcga caacagcgca ttccccctat atgacgcacc cttcagtaca |
| 3361 | agcccaccac caacagcagt tgtaaggctg ccctggagga accgaaaggc caaattccct |
| 3421 | cctcccttct actgcttcta ccaactggaa gcacagaaaa ctagaatttc atttattttg |
| 3481 | tttttaaaat atatatgttg atttcttgta acatccaata ggaatgctaa cagttcactt |
| 3541 | gcagtggaag atacttggac cgagtagagg catttaggaa cttgggggct attccataat |
| 3601 | tccatatgct gtttcagagt cccgcaggta ccccagctct gcttgccgaa actggaagtt |
| 3661 | atttattttt taataaccct tgaaagtcat gaacacatca gctagcaaaa gaagtaacaa |
| 3721 | gagtgattct tgctgctatt actgctaaaa aaaaaaaaaa aaaaaaatca agacttggaa |
| 3781 | cgccctttta ctaaacttga caaagtttca gtaaattctt accgtcaaac tgacggatta |
| 3841 | ttatttataa atcaagtttg atgaggtgat cactgtctac agtggttcaa cttttaagtt |
| 3901 | aagggaaaaa cttttacttt gtagataata taaaataaaa acttaaaaaa aatttaaaaa |
| 3961 | ataaaaaaag ttttaaaaac tgaaaaaaaa aaa |

In various embodiments, the ATXN2 gene product is an ATXN2 mRNA isoform comprising NCBI Reference Sequence NM_001310123.1 (SEQ ID NO: 149358). In various embodiments, the ATXN2 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with an ATXN2 mRNA isoform, comprising NCBI Reference Sequence NM_001310123.1 (SEQ ID NO: 149358).

In various embodiments, the ATXN2 gene product is an ATXN2 mRNA isoform comprising NCBI Reference Sequence NM_001372574.1 (SEQ ID NO: 149359). In various embodiments, the ATXN2 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with an ATXN2 mRNA isoform, comprising NCBI Reference Sequence NM_001372574.1 (SEQ ID NO: 149359).

In various embodiments, the ATXN2 gene product is an ATXN2 mRNA isoform comprising NCBI Reference Sequence NM_002973.4 (SEQ ID NO: 149360). In various embodiments, the ATXN2 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with an ATXN2 mRNA isoform, comprising NCBI Reference Sequence NM_002973.4 (SEQ ID NO: 149360).

In various embodiments, the ATXN2 gene product is an ATXN2 mRNA transcript variant comprising NCBI Reference Sequence NR_132311.2 (SEQ ID NO: 149361). In various embodiments, the ATXN2 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with an ATXN2 mRNA transcript variant, comprising NCBI Reference Sequence NR_132311.2 (SEQ ID NO: 149361).

### SOD1

In some embodiments of the disclosure, the SOD1 gene product is a SOD1 mRNA or SOD1 pre-mRNA comprising sequences from a SOD1 gene sequence (for example the SOD1 gene sequence of NCBI Reference Sequence NG_008689.1 (SEQ ID NO: 167802), the SOD1 gene sequence of NCBI Reference Sequence NC_000021.9 (Reference GRCh38.p13 Primary Assembly) (SEQ ID NO: 167803), or a SOD1 coding sequence), or a portion thereof. In some embodiments of the disclosure, the SOD1 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a SOD1 mRNA or SOD1 pre-mRNA sequence (for example the SOD1 gene sequence of NCBI Reference Sequence NG_008689.1 (SEQ ID NO: 167802), the SOD1 gene sequence of NCBI Reference Sequence NC_000021.9 (Reference GRCh38.p13 Primary Assembly) (SEQ ID NO: 167803), or a SOD1 coding sequence), or a portion thereof.

| Reference Sequence NG_008689.1 | |
|---|---|
| (SEQ ID NO: 167802) | |
| 1 | tccactgggt gacgccagct gggctcctga gtttagtggg gacctgcaga acctttatgt |
| 61 | ctagctaagg gattgtaaat gcaccaatcg gcaccctgtg tctagctcag ggtttgtgaa |
| 121 | tgcacgaatc gacactctgt atctagctac tctggtgggg aggtggagaa cctttgtgtc |
| 181 | tagctcaggg attgtaaaca caccaatcag cgccctgtca aaacagacca ctcggctcta |
| 241 | ccaatcagca ggatgtgggt ggggccagat aaaagaataa aagcaggctg cctgagccag |
| 301 | cagtggcaag ctgcttgggt ccccttccat actgtggaag ctttgtctct ctttgcaata |
| 361 | aatcttgctg ctgctcactc tttgggtcca cactgccttt atgagctgta acactcacca |
| 421 | cgaaggtctg cagcttcact gctgaagcca atgagaccac gaaccccccg ggaggaatga |
| 481 | acaacttgag acgcgctgcc ttaagagctg taacactcac cgcgaaggtc tgcagcttca |
| 541 | ctcctgagcc agcgagacca caaacccacc agaaggaaga aactccgaac acatctgaac |
| 601 | atcagaagga acaaactccg gacacgccac ctttaagaac tgtaacactc accgcgaggg |
| 661 | tccacggctt cattcttgaa gtcagtgaga ccaagaaccc accaattccg gacacaccag |
| 721 | ctactaggga ggctgaggtg aggggatcac ctgaatccag gaagtcgagg gtgcagtgag |
| 781 | ccgagatcat gccgttacac tccagcctga gtgacagagt gagatcttgt gtcaaaaaaa |
| 841 | cagaacaaaa taaaacaaga acgttgaata ggctgtgtgt taattctgag atggagtcca |
| 901 | gacttaagaa tgatcctgag gcttagaccc atatccaaga ggatgattcc agcctggcac |
| 961 | ttataggttc ttcaggcact tcagatgcac cctgtcctga attaagccct ttgtccttga |
| 1021 | acctgctttc cttggtaaac agcaccaagg gggctagaaa cctgggggct gcccttacct |
| 1081 | tccctctctc accatgcacc tctaatgggt ggctggactt gtggttgcat ttccgtgatc |
| 1141 | tcccagcttt cttccagtga tggccttagg tgggctctta tcctgtctcc caaatggcct |
| 1201 | ctctgccttg agtctcaccc catctgctct cagtccaccc actgagctat tccagagacc |
| 1261 | attctgactc cctgtggaca cccctctggt ggcttctcct ctctgtcagt tagaaattgc |
| 1321 | ttttgactgc atgtaacaaa tgtggttacg ctatttaacc aaataaaggt ttatttttct |
| 1381 | cgtgatgaaa agtacacaga taagcagcct acttccatcg ataaccattg agggtttctt |
| 1441 | atgactctta cagcaatgaa atgtgtattt acagtataaa gatatacaat tttaaggttt |
| 1501 | taatataatt gggcctaagc acagtatttt ttgttttagt tttcaactgc ccaggtgatt |
| 1561 | ctattgtgca gccagggttg ataaacactg catctaggcc cagtgccttt gatttgcagc |
| 1621 | tgaggtctag agagaattat tgaaactgca gtcccagcta cttgggaggc tgaggttgga |
| 1681 | gaattgccta agcccagcgg ggtgaggctg tagtgagtca agactgcacc accgcactcc |
| 1741 | agcccgggcg acagagcaac actccatcta aaaaaaaaaa aatttctggt ctaccttttt |
| 1801 | gcccaccttt taaccacaga ggtccccaaa catgaatttt cactctcagg aaggctgaaa |
| 1861 | tgacacatta caagcggttt agtcatctgc acgtgccgcc atctgattta aggcatctat |
| 1921 | tcctcctcac cattcctgac aaagttgaac tgtctacatg tgccacattc tcctctttct |
| 1981 | ctaattcctt ccctccttcc ctcctccctt tcttccctcc ctccctcctt ccttcttctt |
| 2041 | tctctttctt tttctttctc tctttctctt tcuttctctt tctctctcac tctcttttct |
| 2101 | tctttctgac agggtttgcc tgttctgctt ctttaaaccc tggggttcaa gtgcaaaggt |
| 2161 | ccctctcatc tgatttccct attttatttt atttaagtta gatactccac gatgatgatg |
| 2221 | atgattattg ttgagacagg atttcattct gttgcccagg ctggagtgca atggtgcgat |
| 2281 | cacaactcac tgcagcctca acctcctggg cccacgcgat cctcctacct cagcctccca |
| 2341 | agtagcttgg actacagggg tgtaccacca tgcccggcta attacatgtg ccagattcaa |
| 2401 | cccacgttcc agggttccaa acaaccaagg ctctatcatc ctgtcttaac aaatatacct |
| 2461 | tcataaagac ctggaaggca ggttgaaatg tggaattctt ggacttcttt cagttctacg |
| 2521 | tagaagggta gcagtggaga gctggctgcc tgcccgcccc tggcctttcc tcctttcccc |
| 2581 | acccctgctc cttgtggcac tgggcagggc ctacgttcaa ggctagcagt gtactcaaca |
| 2641 | agggacagtc cctgtgtggc ctgttcagga tcctatacac aggctcaggg ctctttgggc |
| 2701 | aggaaattcc aggatccggg tactcaagtg gtgttctaaa cagtggttct tggccgggtg |
| 2761 | tggtggctca cacctgtaat cccagcactt tgggaggccg aggctggcag atcacttgag |
| 2821 | gtcaggagtt cgagaccagc ctggccaaca tggtgaaacc ccatctctac taaaaataca |
| 2881 | aaaacttagc caggcatggt gtgcacgcct gtagtcccag ctactcggga ggctgaggca |
| 2941 | ggagaatcac ttgaacccag caggcggagg ttgcagtgag ctgagattgc accactgcac |
| 3001 | tccagcctgg tgacagagtg agactccata tcaaaataaa tacataaata aataaaaaca |
| 3061 | gtgattctta actgggagtg atttggcaac gtctggaatt atttttggct atcccagcct |
| 3121 | ggcagggagg gacagggtat tactggcatc tagtgagtag gggctaggga ttctactgaa |
| 3181 | catcctacag tgtacaggac agcctccaca gcaaagaact gtctggccca aaatgtccat |
| 3241 | agtgcccaca ttcgatgccc tgcattagga agatataaat actcttaaat atcacagagt |
| 3301 | taaattcctt acccctgttc tagcagagat gatattcttg cggggggagc atcttcttgg |
| 3361 | cttcaacaca ttcttttctc catgggagat gatgccagaa gagggacaga acagggccca |
| 3421 | gtaaagcatg gggcctgggg ccagggaccc ccttgttcag gtgtgacgac catcctacga |
| 3481 | aggcaccacc caggcatcat tagaccgtct caaaagaaga gtaattcact gtcccaaagc |
| 3541 | agctctctcg tgtctgtggg cggatccctt ggcaagttta caatgaactg aaatctgccg |
| 3601 | aacttcctgg aacccaaaga aactttagcc ttgggcaaag gccctttggc cagcatttgc |
| 3661 | actgtttatg caaccgttta gaatatacga attatctgga gactactacc aaatacaaca |
| 3721 | ggcaaaactg caaatatgta tacttcctag aggatgataa aaaaatgtga attgtatttc |
| 3781 | tctgatagag gatgcattag agtctgaggg tctaaatagc gtaaataata aataagtaaa |
| 3841 | taaatcgata gtagtgtact ccaaacgagg ctggaatagc ttctattgct gtttcacact |
| 3901 | ggacttcaat taagtctcag tattttgcca tactcaatat taagtactag gctggacgtg |
| 3961 | gtggctcatg tctgtaatcc cagcactttg ggaggccgag gtgggtagat ggctggcttg |
| 4021 | agctcaggag tttgaaacca gcctgggcaa catggtaaaa ccccatctgt acccaaaata |
| 4081 | caaaaatcag ccaggtgtgg tggcacatgc ctgtggtccc aggtacttgg gaggctgagg |
| 4141 | caggaggatg gcttgaaccc aggaggtgga ggctgcagtg agctatgatg gcgccactgc |
| 4201 | actccagcct gggtgacaga gcgagaccct gtctcaaaaa tcaaacaaac aaccccctcg |
| 4261 | ccccggacaa aagtagtttg cactattttc tcatttcaca atatgtttct gaaatatttc |
| 4321 | ccttgaaagg taagtcatat ttatcattcc tgutgtatgg aggcatcata aattatttca |
| 4381 | ccattctacc ctccttgagt gttgtggcct ttaggccaga caaaaacgca ggtgatgcct |
| 4441 | agaagccaac tagttgccgt ttggttatct gtagggttgt ggccttgcca aacaggaaaa |
| 4501 | atataaaaag aataccgaat tctgccaacc aaataagaaa ctctatacta aggactaaga |
| 4561 | aaattgcagg ggaagaaaag gtaagtcccg ggattgaggt gtagcgactt tctataccct |
| 4621 | cagaaaacta aaaaacaaga caaaaaaatg aaaactacaa aagcatccat cttggggcgt |
| 4681 | cccaattgct gagtaacaaa tgagacgctg tggccaaact cagtcataac taatgacatt |
| 4741 | tctagacaaa gtgacttcag attttcaaag cgtaccctgt ttacatcatt ttgccaattt |
| 4801 | cgcgtactgc aaccggcggg ccacgccccc gtgaaaagaa ggttgttttc tccacatttc |
| 4861 | ggggttctgg acgtttcccg gctgcggggc ggggggagtc tccggcgcac gcggcccctt |
| 4921 | ggccccgccc ccagtcattc ccggccactc gcgacccgag gctgccgcag ggggcgggct |
| 4981 | gagcgcgtgc gaggcgattg gtttggggcc agagtgggcg aggcgcggag gtctggccta |
| 5041 | taaagtagtc gcggagacgg ggtgctggtt tgcgtcgtag tctcctgcag cgtctggggt |
| 5101 | ttccgttgca gtcctcggaa ccaggacctc ggcgtggcct agcgagttat ggcgacgaag |
| 5161 | gccgtgtgcg tgctgaaggg cgacggccca gtgcagggca tcatcaattt cgagcagaag |
| 5221 | gcaagggctg ggacggaggc ttgtttgcga ggccgctccc acccgctcgt ccccccgcgc |
| 5281 | acctttgcta ggagcgggtc gcccgccagg cctcggggcc gccctggtcc agcgcccggt |
| 5341 | cccggcccgt gccgcccggt cggtgccttc gcccccagcg gtgcggtgcc caagtgctga |
| 5401 | gtcaccgggc gggcccgggc gcggggcgtg ggaccgaggc cgccgcgggg ctgggcctgc |
| 5461 | gcgtggcggg agcgcgggga gggattgccg cgggccgggg agggcggggg gcgggcgtgc |
| 5521 | tgccctctgt ggtccttggg ccgccgccgc gggtctgtcg tggtgcctgg agcggctgtg |
| 5581 | ctcgtccctt gcttggccgt gttctcgttc ctgagggtcc cgcggacacc gagtggcgca |
| 5641 | gtgccaggcc cagcccgggg atggcgactg cgcctgggcc cgcctggtgt cttcgcatcc |
| 5701 | ctctccgctt tccggcttca gcgctctagg tcagggagtc ttcgcttttg tacagctcta |
| 5761 | aggctaggaa tggtttttat atttttaaaa ggctttggaa aacaaaaata cgcaacagag |
| 5821 | accgtttgtg tgacactttg cagggaagtt tgctggcctc tgttctaggt catgattggg |
| 5881 | ctgcaagggc agagaaggta gccttgaaca gaggtccttt tcctcctcct aagctccggg |
| 5941 | agccagaggt ttaactgacc cttttgggga tttttgaggg cagtgatctt aactttgggt |
| 6001 | gcacagttag cttatttgaa gatcttacta aaaatacacc agagcccaac ctccgaccaa |
| 6061 | ttacatcaaa acctgtccta gtgcagggtg agtattgctg ttttttgaaa gtttccaaaa |
| 6121 | gtgattttga tgtgcaccta cgattgagaa ctgtcgtttg aggacagtgg gtggagtttc |
| 6181 | gtatttggaa attagaagac ctggagtttc cattacaccg aattggcact taataactgt |
| 6241 | tgtcggagca tttcttaagc cacattttcg taaagtggct ttaaaattgc tctgccagta |
| 6301 | ggcaggttgc taagatggtc agagacaaac ttctgaacga ctcttgtaaa atatacagaa |
| 6361 | atattttcag aacttttatc agtaaaatta caaaacgtgt tgcaaggaag gtgcttgtga |
| 6421 | taacactgtc cccagaacct tagtgaagtt accaactggt ggaaaatttt ctcttgcact |
| 6481 | cggcttaaaa atcatgaggg aatatttact atacgaatga gattcagtct ttaaaggggt |
| 6541 | ttacagaaac gtgagaggac aggaacagtt agtctgtgta aatgtctgaa atatatgtga |
| 6601 | gggagataat gagtttagcc tttttcttta ataggtctcc agattttctg gaaaaggttc |
| 6661 | tttggcattt gactccattt tgctgtttca tttgtcagac ttctttttgt ccctctttac |
| 6721 | ttctccccac ataattcacc agtactagtg ttttgttttt cagaccaagt ctcgctctgt |
| 6781 | cgcccaggct ggagtgcagt ggcgcgatct cagctcactg caacctccgc ctcccaggtt |
| 6841 | caagcaattc tcctgcctca gcctcccggg tagctgggac tacaggcgcg cgccgccacg |
| 6901 | cctggctaat tttttatatt ttagtagaga cggcgtttca ccatgttggc caggatggtc |
| 6961 | tcgatctgtt gacgtcgtga tccacccgcc tcggcctccc aaagtgctgg gattacaggc |
| 7021 | gtgagccacc ccgcccggcc accagtgcta ttcttaagac gcctctgagg aatcccttct |
| 7081 | ccctggccat tgagaatcca tgcatgaacc caggttttcc accttccctg agcagcttgc |
| 7141 | atagttcctt cttttaagcg cctgacttcg ttttgtttgg tgcccgttgt acctgagaat |
| 7201 | gagccttgga tagtggagca ttccagcttt ccagatatgc agagataata cattggctat |
| 7261 | cagctacttg gcttggccta ttccgtgttt aaaatcttgg actctttgct agtttttaca |
| 7321 | gatcagaatt tttcacgtat taatccagtt ttcctagctt ctcttgaaga atttttggag |
| 7381 | atctcttcat actgagcctt cattagccca ggacagtact gctgtagcag ttcatatatt |
| 7441 | ttttcgcttc ccaggcctgt gttattcact taagttcata gcctggtccc tgcagggttg |
| 7501 | tacccgagca cagctactta gatgtcctga atgtattacc ggttaaatgg aggtttcaaa |
| 7561 | gaacctgctg tttttggccc tgtgctcttg ataacagagt gtttgaggga caactttcac |
| 7621 | atttgagttt ttccaaaatt aaaggttgta gaagagtcac agtatctatt gtcaaaaaga |
| 7681 | aaagaattta aaaaggcagc aattgccagg atacttcatt tgagcaatga tattttccag |
| 7741 | tggaaagtca catcttaagg gttaatgccc cttaactgtt ggccgtattt gaaaacaaac |
| 7801 | caagctaaaa acaagagaca ctgacatgtt gtatgacggt gtggtgtgga tgttgtgttt |
| 7861 | attttagtcc tgagatctag ttgtaacttc cttgatttct gtatgtagcc acggagcacc |
| 7921 | attacctgtc accattacct gaatggctat actgcttgct ttcattttgg tagagtggaa |
| 7981 | aggttaccta ggtttcagtg cttgaaaaga tttcagaaag cagtagtacg tctggttaga |
| 8041 | ctagaatcag tcctctcctg ggggcagtgg aatataatat tttctgactg ctaattaaaa |
| 8101 | atacctgtga tagccgggcg tggtggctta cgcctgtaat cccagcactt tgggaggccg |
| 8161 | agacgggtgg atcacgaggt cagcagatgg agaccatcct ggctaacacg gtgaaacccc |
| 8221 | gtctctacta aaaatgcaaa aaaattagcc gggtgtggtg gtgggcgcct gtagtcccag |
| 8281 | ctactcagga ggctgaggca ggagaatggc atgaacctgg gaggcggagc ttgcagtgag |
| 8341 | ccgagatcat gtcactgcac tccagcctgg gcgacagagc gagactcgtc tcaaaaaaaa |
| 8401 | aaagaaaaaa acttatgatg gacacttaaa aacactcact gagtggggag tggagagcag |
| 8461 | gggtcccagg gtagcctgtt ggacatttcc agggcgactt tttctttttt tttttttaaa |
| 8521 | gtcaagtgag tatgccatat ggaaaagggt gtgcgtggag aaaaagcaag gggctccaga |
| 8581 | gtgtaggatg agacatacac cttttgggtt aaaaaggctg aggcaggaga atggcgtgaa |
| 8641 | cccgggaggc ggagcttgca gtgagctgag atcatgccac tgcactccag cctgggcgac |
| 8701 | agagcgagac tcttgtctca aaataaaaaa cgcttacatg tacatgtata ttcaacatgt |
| 8761 | acaaatataa cctattcaaa agtatttact acataaatag gtacttacat tacctattta |
| 8821 | ctgtaatagt caaagcctat gaagtatcta acactgatgt gtaggtactc actttgcttg |
| 8881 | ccactctatt aggtgctttt tatgttattt aatcatgaag cctggccaca gggtgcttgt |
| 8941 | gcattgagtg tgggaacaag attaccatct cccttttgag gacacaggcc tagagcagtt |
| 9001 | aagcagcttg ctggaggttc actggctaga aagtggtcag cctgggattt ggacacagat |
| 9061 | ttttccactc ccaagtctgg ctgcttttta cttcactgtg aggggtaaag gtaaatcagc |
| 9121 | tgttttcttt gttcagaaac tctctccaac tttgcacttt tcttaaagga aagtaatgga |
| 9181 | ccagtgaagg tgtggggaag cattaaagga ctgactgaag gcctgcatgg attccatgtt |
| 9241 | catgagtttg gagataatac agcaggtggg tgctgtgctg tgctggtgac ccatacttgt |
| 9301 | tcaccctagt tagataaaca gtagagtagc ccctaaacgt taaaacccct caacttgttt |
| 9361 | ttgtttttga gaaagggtct tgctctgtcg ctcaggctgg agtgcagtgg cgctgtgcga |
| 9421 | tcatggctga ccttagcctt gacctcccag gctccattga tcctcatgcc ttggcccgta |
| 9481 | gctgggacta caggtacaca ccaccacgcc tggctaattt ttgtattttt ttctagaggt |
| 9541 | ggggtttcat catgttgccc aggctggtct tgaactgctg ggctcaagtg gtctatcctc |
| 9601 | ctcgacctcc caaagtgctg ggattacatg tgtgagccac tgtgcctggg aaaaccctca |
| 9661 | acttttcttt taaaaaagag gtcaacttta ttgtatataa gcactgtgct aaaattgcag |
| 9721 | gaactgggac catatcctga tttttgtaat aatgccagca gagtacacac aagaaaagta |
| 9781 | actgcactag attgtgaaga ctggggtgga cctgcttctg aaggtccagt gccctttgtc |
| 9841 | ttaagatttg gtgtagtgtg tctttagaaa ccaaaaaaag agaagaagat caaccttaag |
| 9901 | attagccaca aaactgggct ttgataccta ggtgtggaaa agaaagggaa agagttgatg |
| 9961 | ttttgtctta cagcatcatt gtagaagagg gtgttttttt gtttgtttgt tttttgagac |
| 10021 | ggagtcttac tctgtggccc aggctggagt gcagtggcgc gatctcggct cactgcaagc |
| 10081 | tccgcctccc gggttcatgc cattctcctg cctcagcccc ctgagtagct gggactacag |
| 10141 | gtgcccgcca ccccgcctgg ctaatttttt gtatttttag tagagacggg gtttcactgt |
| 10201 | gttagccaag atggtctctc tcctgacctc gtgatccgcc tgtctcagcc tcccaaagtg |
| 10261 | ctgggattac aggcatgagc caccgcaccc agccagaaga gggtgtttct taaagaaggc |
| 10321 | aaataggaaa taaaaacttg ggctcttaac ttttgtaatg atcccaggtg tttgagctgg |
| 10381 | gggttgaggg tgggtgcctc gagcaaaggg gctgcattta tttgcataat gccatgtaag |
| 10441 | agtagctcta caccccaaac acaggcttct tagtgggacc aaagtatgat acaaactgaa |
| 10501 | gatggaatgc agaggattat tggtactttg gaatatgctt aaaaaaaatt tttttaaagt |
| 10561 | atttttaaaa aatcaggcaa cccctgaacc agagtaggtt cagagaaact gccaaatttt |
| 10621 | attttcttaa tttgggattg gaagcaagtt aacagaagtt tatgagttaa gttgcattta |
| 10681 | gtgatctttt gccatatttg agtaataatc tgattttttt gtttatagat ttcttcttaa |
| 10741 | attaacttta ttcatcttgc taatttagtt tcaaatagtg atttgtaatg atcagatttg |
| 10801 | atccatttct gtaattgctg aaattccccc gagttgcttt ttggctttac cgcctctggt |
| 10861 | ctgggaggtg attgctctgc tgcttcctgt aacttgcctg cctttctccc tgtgtgggac |
| 10921 | tcctgcgggt gagagcgtgg ctgaagacag ccgtgttatg aaagggcctc ctgtgctgtc |
| 10981 | gaggttgtgc tctgtgaatg tcatcccctg gtgcacagca gcaccttcta cacaggatac |
| 11041 | agttggaatg ccgccccctc gagttgtgta aggcagcagc cttggcccut gcacataaga |
| 11101 | tgctgttgaa tattctgcct gcaccaagta aagggcacag atagaactgc ttggcatatg |
| 11161 | ttgctgggga gatgagtttt ttgtaaagta tactacgttc ttaagaattt ggatcataac |
| 11221 | catgggattt taataataga aaaactgttg aagatcagtc tggtccctta tttttacagt |
| 11281 | gaagaagcca aagcccagag aagggtgtta actttacaag tgtcagacag tagttagaac |
| 11341 | ttggtggggt tttttttttt ttttttttga gatggagtct tgctctgttg cccaggctgg |
| 11401 | agtgcagtgg tgcgatctca gctcactgca acctctgcct cccaggttca agcgattctc |
| 11461 | ctgcctcagc ctactaagta gctgggacta taggtgcgca ccaccacgcc tagctaattt |
| 11521 | ttgtattttt tcagtagaga cagggttttg ctatgctggc caggctggtc tcaaactcct |
| 11581 | gacctcagat gatccagcca cctcagcttc ccaaagtgct ggggttccag gtgttagcca |
| 11641 | ccatgcctgg ccatagactt gtttctgttc ccttctcact gtggctgtac caaggtgttg |
| 11701 | cttatcccag aagtcgtgat gcaggtcagc actttctcca tgggaagttt tagcagtgtt |
| 11761 | tctttttaga atgtatttgg gaactttaat tcataattta gctttttttt cttcttctta |
| 11821 | taaataggct gtaccagtgc aggtcctcac tttaatcctc tatccagaaa acacggtggg |
| 11881 | ccaaaggatg aagagaggta acaagatgct taactcttgt aataatggcg atagctttct |
| 11941 | ggagttcata tggtatacta cttgtaaata tgcgctaaga taattccgtg tttcccccac |
| 12001 | ctttgctttt gaacttgctg actcatctaa acccctgctc ccaaatgctg gaatgctttt |
| 12061 | acttcctggg cttaaaggaa ttgacaaatg gggacactta aaacgatttg gttttgtagc |
| 12121 | atttattgaa tatagaacta atacaagtgc caaaggggaa ctaatacagg aaatgtcatg |
| 12181 | aacagtactg tcaaccacta gcaaaatcaa tcatcattgt gaaacatagg aagcttctgt |
| 12241 | agataaaaaa aaaaattgat actgaaaact agtcgagact ccatttatat gtgtatgttt |
| 12301 | tctgaaagcc tttcagaaaa atattaaatt taaggacaag atttttatat cagaggcctt |
| 12361 | gggacatagc tttgttagct atgccagtaa ttaacaggca taactcagta actgagagtt |
| 12421 | taccctttgg tacttctgaa atcaggtgca gccccatctt tcttcccaga gcattagtgt |
| 12481 | gtagacgtga agccttgttt gaagagctgt atttagaatg cctagctact tgtttgcaaa |
| 12541 | tttgtgtcta ctcagtcaag ttttaattta gctcatgaac taccttgatg tttagtggca |
| 12601 | tcagccctaa tccatctgat gctttttcat tattaggcat gttggagact tgggcaatgt |
| 12661 | gactgctgac aaagatggtg tggccgatgt gtctattgaa gattctgtga tctcactctc |
| 12721 | aggagaccat tgcatcattg gccgcacact ggtggtaagt tttcataaaa ggatatgcat |
| 12781 | aaaacttctt ctaacataca gtcatgtatc ttttcacttt gattgttagt cgcggtttct |
| 12841 | aaagatccag ataaactgta cttgcagttc aaattaggaa aagcaatttt attggacaat |
| 12901 | tacggtgaaa atgaattatt ttatctaggt cagttaagaa cactgttctg ctaagatgca |
| 12961 | gtaaaaagca ggttacattt gaccatatta gatctgagtt tggaaaacag aagtagtctt |
| 13021 | tagttttaaa atggccagat tttcttgcca ggattgggtt tctcacttgt taaacagaac |
| 13081 | attttgttaa gtttaaaacc tgggatggac ttaagtattc atgttcattc atgttcattc |
| 13141 | aggactgcag gttatcatga cttgtttaac ttgtgggaag ctgttgtccc aagttatcct |
| 13201 | ggggaactgc atctggttct tgcaaaacac caagtagaca ggctctcttt tacctcccct |
| 13261 | tgagggcatt aacattcagt agtcacttcc attcagttaa ccctttattt ttatggtttt |
| 13321 | tcttgagcca tagttgtaaa gcagaaaaat catttataaa ggtttgttga acaaaattca |
| 13381 | aaatactgtt gcttaaagta ttaagatttt ttaggattat accttactta taggcccgtc |
| 13441 | attcatttgg catgaaattt tgagttttat tcactttcac tttccttttt ttccaaagca |
| 13501 | attaaaaaaa ctgccaaagt aagagtgact gcggaactaa ggttactgta acttaccatg |
| 13561 | gaggattaag ggtagcgtgt ggtggtctac aacatagtta tttgggtttt agtatttcat |
| 13621 | ttagacagca acacttacct aatgtttaaa ggtaatgtct ttgcaacacc aagaaaaagc |
| 13681 | tttgagtagt agtttctact tttaaactac taaatattag tatatctctc tactaggatt |
| 13741 | aatgttattt ttctaatatt atgaggttct taaacatctt ttgggtattg ttgggaggag |
| 13801 | gtagtgatta cttgacagcc caaagttatc ttcttaaaat tttttacagg tccatgaaaa |
| 13861 | agcagatgac ttgggcaaag gtggaaatga agaaagtaca aagacaggaa acgctggaag |
| 13921 | tcgtttggct tgtggtgtaa ttgggatcgc ccaataaaca ttcccttgga tgtagtctga |
| 13981 | ggccccttaa ctcatctgtt atcctgctag ctgtagaaat gtatcctgat aaacattaaa |
| 14041 | cactgtaatc ttaaaagtgt aattgtgtga ctctttcaga gttgctttaa agtacctgta |
| 14101 | gtgagaaact gatttatgat cacttggaag atttgtatag ttttataaaa ctcagttaaa |
| 14161 | atgtctgttt caatgacctg tattttgcca gacttaaatc acagatgggt attaaacttg |
| 14221 | tcagaatttc tttgtcattc aagcctgtga ataaaaaccc tgtatggcac ttattatgag |
| 14281 | gctattaaaa gaatccaaat tcaaactaaa ttagctctga tacttattta tataaacagc |
| 14341 | ttcagtggaa cagatttagt aatactaaca gtgatagcat tttattttga aagtgttttg |
| 14401 | agaccatcaa aatgcatact ttaaaacagc aggtctttta gctaaaacta acacaactct |
| 14461 | gcttagacaa ataggctgtc ctttgaaagc tttagggaaa tgttcctgct tagtcatttt |
| 14521 | agcattttga ttcataaagt acctcctcat tttaaaaaga cattatgatg taagagagcc |
| 14581 | atttgataac tttttagtga gctttgaaag gcaagttaca gcctcagcta gctagtaaga |
| 14641 | ttatctacct gccagaatgg cacaaattct acattcaagg gtagacgctg gcacaaccta |
| 14701 | cttacagatt agccctttaa agcaatctgt agcattagaa gatggaacca aggaaatgtt |
| 14761 | tgactgtggg ttctggctgt tgagaaataa tttacacacc gaattagtga aatgagtcac |
| 14821 | tttctcttaa tgtatttatg tacctgagag aatgcttttc aatgttaacc taactcaggt |
| 14881 | ttgactaaat tattcaattg gaaattgtag aatattattt ctgataaacc agaaataagt |
| 14941 | gaaatgctgt ttgttcataa atatgtactt tatcaaatgt aggagagatc atttaggaga |
| 15001 | ggaaaagcta aattggaaga caaatctgta gtgtttccaa agttttaaaa ttatggtaaa |
| 15061 | caacagtatg ttcacagtaa gtggttaaaa caaccattct ttaaatctca gtagagaatt |
| 15121 | tttaaaaagc agtatttaac acatttccct aatgtagttt gttgcctatg tggaataact |
| 15181 | caattagaga ctcacttatg ccttttgaaa cttcaaatat aattacacta ccagttttta |
| 15241 | catgtgcata taggatggtc ccaatacttt aaattggaaa tacaggctgt aagtccttca |
| 15301 | agtctggatg ttgggtaatc acgttttctt ccagaagcca tttgttagga ctttaaaact |
| 15361 | tctcagtggg ccagtgtaaa attaaggaca agctttataa tttaaattta cagataaata |
| 15421 | taaaacaatt ttctctcatt ttctaaaggc aggaatataa ggacattgcc ctagatatat |
| 15481 | tctccccatc aaaccaaagg tcttgctgct gagtattatt taaaaaccag agtaataaag |
| 15541 | cagcttcgcc tgaggatgaa ggcaaagaca aagttaactg ctcagcatac tgaaaccagg |
| 15601 | agtttgaaaa agtaattcaa aacaacttca aggccataaa tacttggata gtgtgacatc |
| 15661 | aggtcttggc actggatttc ctgcaatttc agatgaaggc ttgacttggc tttagatgtt |
| 15721 | ttcatgtgat aattcttgga taacctacaa cgatttttcc tatttaaccc accaacttta |
| 15781 | agcaaaagaa agaacgtata tgtgggggaa aagttcgttt acctctgccc cgcaggggaa |
| 15841 | ttaaaacgct ggtgaagaaa ggcaacaggc aaggcttata taaggaaaca gtagtaggct |
| 15901 | gcactgaagt cctcaaacac aacaaaaagc tcttaggttg caaatgttaa ccttgattct |
| 15961 | tttacccttt tgaaaaattc aatgggatgg ttggaaaaaa aaaaaaccac agacacacac |
| 16021 | acacacacac gcaaccttct aacgtaatac ccaggcagtc cgataattta tagtacaacg |
| 16081 | tgtaacactg ggatccaact tttcacccag caaaatctcc ttacaaatta gggcaacata |
| 16141 | taccacaaag ccaatggaaa aaaaaagaaa acctcgattg aattgcaaac acagcttttc |
| 16201 | aattgacatt aaaacaacta acctttacct tatgactgag cactaaaaat caaaagtatt |
| 16261 | acatatgaaa gtgagaataa ctacataaaa tgtctatttt catcaaataa |

In various embodiments, the SOD1 gene product is a SOD1 mRNA sequence comprising NCBI Reference Sequence NM_000454.5 (SEQ ID NO: 167804). In various embodiments, the SOD1 gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a SOD1 mRNA sequence comprising NCBI Reference Sequence NM_000454.5 (SEQ ID NO: 167804).

| NM_000454.5 | |
|---|---|
| (SEQ ID NO: 167804) | |
| 1 | gcgtcgtagt ctcctgcagc gtctggggtt tccgttgcag tcctcggaac caggacctcg |
| 61 | gcgtggccta gcgagttatg gcgacgaagg ccgtgtgcgt gctgaagggc gacggcccag |
| 121 | tgcagggcat catcaatttc gagcagaagg aaagtaatgg accagtgaag gtgtggggaa |
| 181 | gcattaaagg actgactgaa ggcctgcatg gattccatgt tcatgagttt ggagataata |
| 241 | cagcaggctg taccagtgca ggtcctcact ttaatcctct atccagaaaa cacggtgggc |
| 301 | caaaggatga agagaggcat gttggagact tgggcaatgt gactgctgac aaagatggtg |
| 361 | tggccgatgt gtctattgaa gattctgtga tctcactctc aggagaccat tgcatcattg |
| 421 | gccgcacact ggtggtccat gaaaaagcag atgacttggg caaaggtgga aatgaagaaa |
| 481 | gtacaaagac aggaaacgct ggaagtcgtt tggcttgtgg tgtaattggg atcgcccaat |
| 541 | aaacattccc ttggatgtag tctgaggccc cttaactcat ctgttatcct gctagctgta |
| 601 | gaaatgtatc ctgataaaca ttaaacactg taatcttaaa agtgtaattg tgtgactttt |
| 661 | tcagagttgc tttaaagtac ctgtagtgag aaactgattt atgatcacct ggaagatttg |
| 721 | tatagtttta taaaactcag ttaaaatgtc tgcttcaatg acctgtattt tgccagactt |
| 781 | aaatcacaga tgggtattaa acttgtcaga atttctttgt cattcaagcc tgtgaataaa |
| 841 | aaccctgtat ggcacttatt atgaggctat taaaagaatc caaattcaaa ctaaa |

### MAPT

In some embodiments of the disclosure, the MAPT gene product is a MAPT mRNA or MAPT pre-mRNA comprising sequences from a MAPT gene sequence (for example the MAPT gene sequence of NCBI Reference Sequence NG_007398.2 (SEQ ID NO: 301567), the MAPT gene sequence of NCBI Reference Sequence NC_000017.11 (Reference GRCh38.p13 Primary Assembly) (SEQ ID NO: 301568), or a MAPT coding sequence), or a portion thereof. In some embodiments of the disclosure, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA or MAPT pre-mRNA sequence (for example the MAPT gene sequence of NCBI Reference Sequence NG_007398.2 (SEQ ID NO: 301567), the MAPT gene sequence of NCBI Reference Sequence NC_000017.11 (Reference GRCh38.p13 Primary Assembly) (SEQ ID NO: 301568), or a MAPT coding sequence), or a portion thereof.

| NG_007398.2 | |
|---|---|
| (SEQ ID NO: 301567) | |
| 1 | gggattacag gcgtgagcca ccacacccag cccagaatgt ttattagaat gcacaattaa |
| 61 | taccagaggc agtggggaag gaaggactga gcagaggagg aagttgagct gtgattcaac |
| 121 | ccaacaactg cctggctggc atggggagct ctggagttaa atagggccat cagactttcc |
| 181 | cagtgtgggg ccaacatgac tgggtcttta tacccccacc tctgtcagtc actcaacgtg |
| 241 | gtctccctgc aacaaggtga ctcttgcagc cgagacaatc cctgaaggga cagagggctg |
| 301 | aagcctgtct gccaacagca ctcccagtgg ctggaacaag tccttcccta taggggaatc |
| 361 | tgggcggcac acctccatct ccatgtccat cacatacgat atcacagaca tttaaatatt |
| 421 | ttgataactg tacataagag tttcctttat aatcttatag atcttatttt atgcatttga |
| 481 | aaatattctt ctgagacagg gcttttatca tattgccata gggtgccacg atataaaaaa |
| 541 | ggttaaatac tctctgattc agaagtatcc aatgatgact tctctctcat gcatttaatt |
| 601 | gaaaatctgg tttttctcct tctctgctag ttctctacct ctctccccac ctcccacatc |
| 661 | atagcctatt cacatatgtc tgaatctcat gatagacaag ttcaggttct tttcccaggt |
| 721 | tctttttacc acatcccccc acccccacat aaaaagtata tatggcacag cctaggttcc |
| 781 | acccaaatcc tttctcctct tcttcctggg cccacaactc tcctacatac attggtatac |
| 841 | cttgcgctta gggatggcca tgtgactaag ttctaacagt ggaacatgat cagatgccac |
| 901 | ttccagcctc taagacagcc agtgtgtttc ctccataagc tccttctctt cctcccaact |
| 961 | ggagactcta aatgatgacc ctgcctcaag caagcaaaca acaagtccct caggggtggt |
| 1021 | gtaggctgca aatggaagga gcttgagtcc caaaccttcc acggagaagg ctggctacca |
| 1081 | acctggatca ctcacccaag actgctcgaa gagttggttt gaaccattgt gttttggggt |
| 1141 | ctatttatta caacagttta gcttgctttg tgaatagatt tagtggcaga gcctccaaat |
| 1201 | tctatagata cattgatctc agtcctaacc gcatctggaa caccattaaa taaaggaatt |
| 1261 | gcaaacccag agaaggtaat gaatttgtct aaggtcatac aagatggcta ggatcaggac |
| 1321 | ccaactctcc agttttcttt cttctctgct attctgcctt ctgtgatcct acataagtgg |
| 1381 | gcatgattgt ataacatatg cggccatgag atttctcttt cagcaagaga aagggacagg |
| 1441 | aagaaagaga gggaatgcat tttcttggcc tgaattagtg tgagccatta gttacctaca |
| 1501 | ttgactaaat tatctggaat gaacattcaa ctctacatca catatagtta aaatgacaga |
| 1561 | tctgcttaag attgtttcta gcatacgtta tttcaattta ggcaaatgtg accattcagt |
| 1621 | gtgaggggac catactgtca ttaggtccct gtcagttctc aattatactg ttatcttaga |
| 1681 | gggggaaaaa tgtgaaattt gaatgtagac gagtgttgat ttgactgcta cagtttattt |
| 1741 | tacgtataga aataaaataa tgtgtagcaa aagcattatt acaaagatga taatgaaata |
| 1801 | actagtattt ataatagtat aatagtatag tatttataat agtatgatag tttaatgact |
| 1861 | atttgtcaga tgttgtgtaa gaaactttat acacacacac acacacacct catttaattc |
| 1921 | ctgtatcaat caggatacag gacgctgtgg taacaactcc tcaaatctcg gtggcttgca |
| 1981 | caacaaatgc ttatttcttt tttttttttg acaccaagtc ttgctctgta acaggctgga |
| 2041 | gtgcaatggt gcaatctcgg ctcactgcag cctctgcctc ctgggttcaa gcgattctcc |
| 2101 | tgcctcagtc tctcgagtag ctgggaacac aggcacgcgc caccacatct ggctaatttt |
| 2161 | tgtgatttta gtagagatgg gatttcacca tgttgctcag gctggccttg aactcctgac |
| 2221 | ctcaagcgat ccacccacct cagcctccca aagtgctggg attacaggca tgagccactg |
| 2281 | cgcccagccc caaatgttta tttcttgctc atgtgacatg tacttcctcg agtttttcct |
| 2341 | tcctgagatc taagctgaag gaacagctct ctggagccac gccattctgg tggcggaaag |
| 2401 | gaagagtaaa agtggtagaa ccttgcaatg ctcttgaagc gcctatttgg aatgtctaca |
| 2461 | tcatgtaaat ggtaatggac aagtatgtat aatccccaca ccaaaaaaag gggacactat |
| 2521 | tggggacaat aaccacattt caatgctgca agacggatat tgactgcacc cccttcccac |
| 2581 | tttcagaaag aagaagagta attttgctga actccttcta gagactggaa atgtcccttc |
| 2641 | cagttggggt gattagggaa ggctttggta aaatttgagc tagagtttga aggttaggta |
| 2701 | gactactggt gggtgaagaa agaacaagga cctttgtagg caaaggaaaa cctcagaatt |
| 2761 | acagaggtgg aaaaagagtt ctagtcaagc cacttcagct ggctacagag taggtgggaa |
| 2821 | agaaaatggg aggacaaggg ctcagatgat ggggggttgg ggcattgggg ggacacttga |
| 2881 | aagctaaact aaggggttga acttaattta ggaggcagtt agaagctttt acatattttt |
| 2941 | gagcaagaga gtgacataat taaaatgatc tgggccaggt gtggtggctc acacctgtaa |
| 3001 | tcccagcact ttgggaggct gaggagcttg ggtcacctga ggtcaggaga tcgagaccag |
| 3061 | cctggccaac atggtgaaat cccgtcctac taaaaataca aaaattagcc gggagtggtg |
| 3121 | gcatatgcct gtaatcccag tagctgggag gctgagacag gaaaatcgct tgaacccggg |
| 3181 | aaacaggttg cagtgagccg agatcgtgcc actgcactcc agcctgggca acagagcgag |
| 3241 | actccatctc aaaaaaacaa aacaaacaca cacaaaaaac caaaaataaa taaataaaat |
| 3301 | gatcacttct gaatactgat ctaactaggg gttgcagggt gggctgatat agggagaaac |
| 3361 | tggagagcaa ggagatcact aaggtcccta catgtccaga accaagatag aggtcttgaa |
| 3421 | ctaggatggt ggcagttaga acaacaacaa caaaaagtca attccaggct gagtgcagtg |
| 3481 | gctcatgcct gtaatcccaa cgctttggga ggctgaggtg ggagttagaa agcagcctgg |
| 3541 | gcaacactgc aagacctcct ctctaaaaaa aaaaaaaaaa aaaagttagc caggtgtggt |
| 3601 | ggtgcccacc tgtagtccca gcaactcaga aggctgaggt gggaagattg cttgagcccc |
| 3661 | aggagttcaa gcttgccgtg agctacgatt gtgccactgc actccagcct gagcaagacc |
| 3721 | ttgtctccaa aaaaaggtca attccactga ctcttctaag gtgtacacca tcaaggggca |
| 3781 | gctccatctc caggccattg gctcatgaga cattctgtag tcagaaggct agggcagatt |
| 3841 | gctttgagca agcccccatg gtggttctca ctcctacttc tttgggtata tgcccctctg |
| 3901 | tttaaaaata aagttaatat gcatttaaaa aaaaaaagga gaaaaaggtc agttccagaa |
| 3961 | actgtgtgaa taaagcattt tacttgcttt ttctattaat ctataacata tgttgatttt |
| 4021 | ttaaaaagaa tataagagct atgcaaattg gagcttcaag acaacttccc atctccctag |
| 4081 | gaggagatgg ctgccctaaa cccccctaca tagaaatcat cccactgctt gggcttaaac |
| 4141 | ttgatgttgg ggaaatgaaa aatccaagct aaggccgaag cctggggcct gggcgaccag |
| 4201 | cagaatgagg accactggtc agtttcaggc tgaggtgcgt cttccagggg acaatctcta |
| 4261 | gctggccctt aaacattcag acttcaagct ctatttacag cataaaggtg tttcaaaaga |
| 4321 | cgtgatacaa ataactgcaa atgctctgcg atgtgttaag cactgtttga aattcgtcta |
| 4381 | atttaagatt tttttttctg acgtaacggt tagattcacg tttctttttt tttaagtaca |
| 4441 | gttctactgt attgtaactg agttagcttg ctttaagccg atttgttaag gaaaggattc |
| 4501 | accttggtca gtaacaaaaa aggtgggaaa aaagcaagga gaaaggaagc agcctggggg |
| 4561 | aaagagacct tagccagggg ggcggtttcg ggactacgaa gggtcggggc ggacggactc |
| 4621 | gagggccggc cacgtggaag gccgctcagg acctctgtag gagaggacac cgccccaggc |
| 4681 | tgactgaaag taaagggcag cggacccagc ggcggagcca ctggccttgc cccgaccccg |
| 4741 | catggcccga aggaggacac ccacccccgc aacgacacaa agactccaac tacaggaggt |
| 4801 | ggagaaagcg cgtgcgccac ggaacgcgcg tgcgcgctgc ggtcagcgcc gcggcctgag |
| 4861 | gcgtagcggg agggggaccg cgaaagggca gcgccgagag gaacgagccg ggagacgccg |
| 4921 | gacggccgag cggcagggcg ctcgcgcgcg cccactagtg gccggaggag aaggctcccg |
| 4981 | cggaggccgc gctgcccgcc ccctcccctg gggaggctcg cgttcccgct gctcgcgcct |
| 5041 | gcgccgcccg ccggcctcag gaacgcgccc tcttcgccgg cgcgcgccct cgcagtcacc |
| 5101 | gccacccacc agctccggca ccaacagcag cgccgctgcc accgcccacc ttctgccgcc |
| 5161 | gccaccacag ccaccttctc ctcctccgct gtcctctccc gtcctcgcct ctgtcgacta |
| 5221 | tcaggtaagc gccgcggctc cgaaatctgc ctcgccgtcc gcctctgtgc acccctgcgc |
| 5281 | cgccgcccct cgccctccct ctccgcagac tggggcttcg tgcgccgggc atcggtcggg |
| 5341 | gccaccgcag ggcccctccc tgcctcccct gctcgggggc tggggccagg gcggcctgga |
| 5401 | aagggacctg agcaagggat gcacgcacgc gtgagtgcgc gcgtgtgtgt gtgctggagg |
| 5461 | gtcttcacca ccagattcgc gcagacccca ggtggaggct gtgccggcag ggtggggcgc |
| 5521 | ggcggcggtg acttggggga gggggctgcc cttcactctc gactgcagcc ttttgccgca |
| 5581 | atgggcgtgt gtgtgtgtgt gtgtgtgtgt gtgtgtgtgt gtgtgtgtgg aggggtccga |
| 5641 | taacgacccc cgaaaccgaa tctgaaatcc gctgtccctg ccgctgttcg ccatcagctc |
| 5701 | taagaaagac gtggatcggg ttctagaaaa gatgactccc tgcacgcccc tccctgcacc |
| 5761 | tcccgagcag tgattccgac agggccttca ctgcccctga ttttaggcgg gggccggccc |
| 5821 | cctccccttt tcctccttca gaaacccgta ggggacattt gggggctggg agaaatcgag |
| 5881 | gagatgggga ggggtccacg cgctgtcact ttagttgccc ttccccctgc gcacgcctgg |
| 5941 | cacagagacg cgagcagcgc cgtgcctgag aacagtgcgc ggatcccact gtgcacgctc |
| 6001 | gcaaaggcag ggttcacctg gcctggcgat gtggacggac tcggcggccg ctggtccccg |
| 6061 | ttcgcgggca cgcacagccg cagccacgca cggatgggcg cggggctgca ggtgcatctc |
| 6121 | ggggcggatt tctttctcag cgctcggagc gcagggcgcc cggcgtgtgc gctccctgcc |
| 6181 | ggaggcgcgg ggctggcgcg cagggctcgc ccctcactgc ggcagtgggt gtggaccctg |
| 6241 | gtgggcgagg aagggggagg ataggctgtg ccccctccca ctcccgcccc cagcccccct |
| 6301 | ttttttcccc ctcggaacgc gaggtgccat ctcttttcgg cgtgtcacgt ctttacggtg |
| 6361 | ccatgccaaa ccgggtggcc gggcttcata ggacagggcg gggcctggca ttaaagggag |
| 6421 | ggggacaatc agcgctgaaa tcttggcgtt ttgctgctgc gggcgtgagc actgggggcg |
| 6481 | ttcgcccagc accttcttcg ggggctcttt gccttgtctg tagaggttac gtgatctgcg |
| 6541 | ctcccagccc tggtttctgg cttttattct gagggtgttc agtcaacctc ccccctacgc |
| 6601 | ccatgcgcct ctctttcctt tttcgctcct catttccgag cccattgtcg gatctcgagg |
| 6661 | cttgctgggt tcgatgaact cgagtcaacc ccccgacccc cggcacgcat ggaacgggcg |
| 6721 | tgaccgcgcg cagcctcgtc tcggagtctg ccggcgccgg gaagcttctg aagggatggg |
| 6781 | attcgagtct ccgtgcgcgc tgcgggcggc ggcagaggga tctcgcccct ccctacaccc |
| 6841 | caagtgtcct gagggccacg ccacaccagg ttgcccagcg agggacgctg gctacccatc |
| 6901 | cggggatggg tggggagccc tggcggggcc tctccggctt tacgccctgt tgcttcgcct |
| 6961 | ggccggagaa tgtgaggaag gggcataagg ttactggtgc ttcggccaca cccatctttc |
| 7021 | tgagcccact ggactgggcg cagagggggg attgccatgg aaaccacagg tgtccggaga |
| 7081 | ggggatcttg gggctggcct caccccttcc ctgcggagat tggggaccct ggggtagggg |
| 7141 | gagccgcgcc cagtcggcct cctggaggac acgggaggaa gccccgaacc cccgcgcctg |
| 7201 | aggctgtttc tgattggccc ctggaggccg cagacacgca gataggcggc cctgggtgta |
| 7261 | tttttattaa tattatgtcc gtactgatta atattattta tcttaaataa atttcacccg |
| 7321 | tgtccaagtt caccgcgccc ccaaaaccga gtctggggcg gcagggggaa ctcctggcca |
| 7381 | acgaatccat gcctcgccct cctgtgatga acctggtacg cacggttttc tggttaattc |
| 7441 | tatcgctgaa aactggtgcg gggggcgcac ttctgagacg gaagagcatc taggagctga |
| 7501 | atcctccacg cgggtcgccc aggttgatct gaatttctgg ggaatggctt ggctgcccgc |
| 7561 | ccgggaccag gccgaccctc cttgacggtg gcgtagaggg ctggagcctg ggtactgcga |
| 7621 | ggctcctcgc atggctgggc ccgccgcgag gggttgcaga gcggctcagg gatcgattca |
| 7681 | agcatcgtct ctcctccctc gcccccagac agagctgggc gcggggttcc ccttccagat |
| 7741 | ggagcgaggg tctcggggtg gccccggaaa aggggagccc gcggccacgg ctacgtattg |
| 7801 | ccatctcgcg agcagagatg tcacctcctg cccttggagg aaagggagcc cggtggggat |
| 7861 | gagcgcattt agcccaatgc tgggaacaaa gcgcactccg cgcttctgcg atttcgctcc |
| 7921 | attttgaaat gtgttggcgc tttggtgggg ccgctgcggt gggcaaggcc gggggcgctg |
| 7981 | ttaatggagg aacctcaggg ggacggtcct tcgtaggaaa ctctatcctg gctctgcgcg |
| 8041 | cgctttaagg aaatggcttc cctccaggac ctcgagggat gcagcttttg cgcggatgac |
| 8101 | ggtggggtgc tgaaccagcc ggtgcgcctc tggaaatgtc tgggcacgga tcctggggcc |
| 8161 | atcgacgact cctccccatt cccagcaggc gggagctctt acattccgag cgagtgaccc |
| 8221 | ctctcaccct ctggcgctca cacacctgta actccaaacc tccgtctcag aatggtccag |
| 8281 | gctggaaggg atgatggggg ctccgacagc gactgcctag ctcacccccc tgcgtgctca |
| 8341 | ggctccaggc tcagcaggac caatttgagt tctatctgat ccccctcggc cccttaactg |
| 8401 | acccatccta caggagacag ggaaatgtct ttcctaccgc ggttgattct ggggtgtcat |
| 8461 | tttgtgtttt gtgatggctg cttatattta ctgtataagc attgtattta ctgtataagc |
| 8521 | attgtattat aattactgta taagctgctt atatttactg tataagcatc tccaaatcct |
| 8581 | ccctctacgt aaacaaatta atggataaac agataagtgt atcccctgcc cccacccctg |
| 8641 | ctacgcaggt ccggagtgac tcttgaagct catacattcc ttggccaagt ttgcttctct |
| 8701 | aacagatgtt tatatagcaa taacctggct tggctcttgg gttcacctct ggacgatttg |
| 8761 | gggaaggggc ttgttggctt tgctgggttt tggatgagtg acagtccatg actgttcctg |
| 8821 | ctggaagggc gtgactttta agtggtttct aatatcaggc attgctcctc cgacaggaac |
| 8881 | aaaagaaatg gatactgccc ataaattgtt agaaaactta gaatcgcttt gattgaggaa |
| 8941 | aggttagatt tattccggtt ggaaaaagtg gcctttctat taaacgtgcc ctttgaccct |
| 9001 | catgcccttg gaggtcggtg ccagcctgga gatgggataa gattgtggct ttccttctgc |
| 9061 | ctttttaaca tctgttgtta cagtccattt gttgaaaatt taaagaaact gttttattcc |
| 9121 | actttccctc agcatttatg tgtgtggttt cagtagctct gtggctatat gtacgaacac |
| 9181 | gtgttatttt tccaattgga catgtgataa ttctccaact ggaccttgcc ttctattgat |
| 9241 | gtatttattt agcatcttcc ttactccctc cttgaaaaag aatcactcaa aaacaaataa |
| 9301 | aaacagccgt aggggcctaa tacagtgcta gacatacaag aggtattcgg tccataccaa |
| 9361 | atggatttta tccatgaagg ataaatgggg aaatacagtg ggaagcaggt gggaaactgc |
| 9421 | gtttgactct gctctttcct ccaccaccac tttcctcatc accgtgttca gagaccccca |
| 9481 | aagccccctc acactcccag aaacaccccc ctggccactc ctaacttgcc atgcccagga |
| 9541 | gttaggtgct tccactagtg acatggagct ggcgtttggg gggcacctca gcaggtgacg |
| 9601 | ggaagagaag accccagcct caccagctgg gctgcagcag ggagaggagt cctcatgttc |
| 9661 | cagcagggac tctcagctgt tttcctgtaa aaccatggtt ctcaactggg ggccactgag |
| 9721 | atgtctagag agatgttttt gttttcacaa ctcggggagg gtgctactga catcttgtgg |
| 9781 | gtagaggcca ggaatgctgt taaacatcct acaaggaagg cacaggacag tctcctacat |
| 9841 | caaaatatga cccagtccca atgtcaccac tgctggggtt gacactggca ctgctatctt |
| 9901 | aattacattc attgagtgtc ttttaggagg ccctattcta agtgcttgct aagattatct |
| 9961 | catttaatcc tcacaacact tccgctatgt agcaggtgct gttattatct ccgtgatggg |
| 10021 | gaaactgaag cacagagagg gttagtaact tgctaaaggt cacagagcca gtgggtggtg |
| 10081 | gagctggttg cctgacacta gttccctccc ctctcagcca catgtgggct tacttggcca |
| 10141 | ttgtggacta gtctgggaac ccagatatga tctataacat tgacccagta gaatattgat |
| 10201 | tccaaaacca ctgtctcaca aatgaatttt tacaagagtc tgtaatcgga gcatgaccca |
| 10261 | gaataaggtt agggagatgt ggagttaaag ctctcaattt cttatctggc cccgacacag |
| 10321 | agagcaaggc atttcactct acattggtgc tctgtttata aaacaaagag caaatatctc |
| 10381 | ttcctaaggt ccttaaacct cttcccccaa tccagggttt ctggactgct ctgccatatg |
| 10441 | acggggcagc tggtttgatt gacccaggga aggctggaaa tcaagactgg gggatcaaga |
| 10501 | cgtagattca gtgtggccaa ggtcaagtct ctgaggttta gggacatcag atccccagct |
| 10561 | taggttctgt acctcggcaa ggtgaaagcg ttggcgccca ctgatgaggc ctgctctgag |
| 10621 | attgtgggtg tgggttgagt tgggtgggca taggcaagtc ctcttgtaag aatcttttgg |
| 10681 | caaagatggg cctgggaggc ttttctcact tcctggggcc caggctttgc aataagtatt |
| 10741 | ccattatact gtggtacctt ggggctacct gagaatcctc tgtctcgccc ctgttgcctt |
| 10801 | gccaaagagt ttgctgtcca agaattcctt tcctgtctcc aggtgccatg ctcctgccac |
| 10861 | ctctgccagg ttccctgcct gcccagatgg ctcccaactg agtgtgagga ggaatttgag |
| 10921 | acaggttttg agctttctgg gttctccagt taggaaactt tctgtaagca tgcagataga |
| 10981 | atgggcttca gcaaaataca aactcgaaca acttccatgt atagtccctt aattttcttt |
| 11041 | gcttttttca tatttcatca ggctccatgc tgagcccaat cagggacccg atagaaatcc |
| 11101 | aaacaccatg tcagcgagtc cccaagaaat gcattttgtg ccaaggctat tcaaggaagg |
| 11161 | tttgggagca gctcaagggc agacactgtt accctccccc aggtccccag tgcagggcag |
| 11221 | tgttctgcat gtggaggcag tttggcctaa tggttaagga ggtaggctct gatcgggcct |
| 11281 | cctgggcaca aatcccagct ccctgctcac tgtgagacct aagccatatt gtttagctgc |
| 11341 | ttggagagtt ttttgtcatc cacaacttgg agtatgatgg tacctgtctc acgggttgcc |
| 11401 | atggggttca cacaagctaa cccggtactc actagggcca agcacatagt aactgctcag |
| 11461 | taaatggcat catcggcggt gtcctgtgga tgagtgcttg tgattggctg aatgaccaga |
| 11521 | ggggtctaaa gatcctggtg atggaatcag ttgtacagat aaattgttac actgagtagg |
| 11581 | gatcaagata ggaaaagtcg gcaactaccc agctcccctg caccaaactg ggcagaagtg |
| 11641 | gatcctctga aaattgcaca cacccatgtt taaatgtaca cacagaactc ttgccacagg |
| 11701 | caagcggaga tttgtcatct gctgtccctg cctcatcttc ttcctgaaat ccactccatg |
| 11761 | ccaggaataa actgcatgct ctccaccagc ccaaactgac ctgccttccc gccagccatc |
| 11821 | ccgggcaggg tgacctggct tagtacatcg ggctcagaga tctttccagt ttactcgttg |
| 11881 | aataaaaagt gagggctgat cgagaaagta atggcagtca gggaaggcga aggaggtaaa |
| 11941 | gaagagattt tacaaatgaa gtaattcaac agagtgctga cattggtaaa ctggcaaaca |
| 12001 | gatttcaggg tggttggttg agagtagagt agaaaaggat taaataaagc aaacttgtgg |
| 12061 | tgtactgaat cttaggaatt ccatgtatcc aataagtata gtcatttatg aattaataaa |
| 12121 | ttcggcctaa gaagccttct tatcgcttaa atcaagacta agtaacaata tatcagtttt |
| 12181 | aaaaagtcat tatatcagaa aatcatttaa atgatacaca tagatttcca agattttact |
| 12241 | ttaaccgaaa ctatataaat gtgaatttgt tcacccatct tttgacacag ggctcaggtc |
| 12301 | ttctcttggt gtctggatca gccagttgaa atttcttgtc tgttttgcct atgccacatt |
| 12361 | aataatgcac tgtctgggtc ctccgatttc agtttggatt ttgggtttac attgtggagt |
| 12421 | catctgaatg cagaatcctt cagggatttt actttttttt ttttttttca tggtctttac |
| 12481 | catcccattt gatagtaaat attactcacc tttatgaagt ctttccaaaa cattcaacta |
| 12541 | aattttctta aaatcattga atgatttgaa gagcttattc ctcagcactt ttactccatc |
| 12601 | agcttgcacc ttatttttta atcttttttt gagacggagt ctcgctctat cgcccaggct |
| 12661 | taagtgcaat ggcgcgatct tggctcactg cgacctccac ctcctgggct caagcaattc |
| 12721 | cgcctcagcc tccgccgtag ccgggactac aggtacacac cataatgctc ggctgatttt |
| 12781 | tgtatttttg tagggatggg gtatcgccat gttggccagg ctggtcccga acttctgacc |
| 12841 | caagtgatcc acccacctcg gcctcccaaa gtgctgggat tacaggtgtg agccaccgcg |
| 12901 | cccggccagc ttgcacctta tttaggatat gtgattatta tagcaagtct ggtgtacata |
| 12961 | caagattttg aatgggcaca gatgaccttt agtaagtgct tggctgtgat aagaggcagt |
| 13021 | cctgactgca gatcaggctg tgtggacccc agccttgcat gtttacagac cttcatgtct |
| 13081 | tattcttaca gggtatcaga agaacaccta ctggggaaac ttataaatta gtaaaaggtg |
| 13141 | ggcattctcc ccgcccatct tctgtctgtc tgccaggact agcacagcac tttgaagtca |
| 13201 | ttcacataga atcccaactt aagagggtaa aatcctcctc aacagactga aaataagttt |
| 13261 | aaattccctt tgctatatta actcccctga ggaaagagtc ttagatcaat gtccaacact |
| 13321 | aaaaacagtt ttaaatcagc aagtgagaat taaatctgaa gcaattgata ataatgtttc |
| 13381 | attcattcct ctcctttggc cccgtccacc ctactgctaa atccaggcat caaagagaag |
| 13441 | agggacataa ttatctctag tcccagctgc tggttttcct tccagcctat ggcccagttt |
| 13501 | tctgttttac tgagaaggct ggtgatgtta tcttgggatc taagtctgca gtttcaccac |
| 13561 | aaaaagtcca gggatgcact ttcatgcttg tgtcctcctc cctgggatag caaggatatt |
| 13621 | agaagacccc tggctctgta attgcttgtc atgtgctcta cagacgccac agaatgccaa |
| 13681 | gaacgaagtg ctgggaagga caaattcatg gaaccgtggg acggtgctcc tcccccagcg |
| 13741 | taaaggacag ctcctcctcc tgaattggag ccagcgttct aaatcatgtg tcaacagagt |
| 13801 | tgtcctggat cggatccagt tctgccattg atttgcaggt catttcagtg gtacctgttt |
| 13861 | ccagttgttc ttaattgaac agtggcacca aactattgtc ttgcctcatc cccctcccat |
| 13921 | ggcctgtccc ccaaaaagag acttcttggg taattaatca gggcaacatc aggcagtctg |
| 13981 | ggcgcggtgg ctcacgcctg taatcccagc actttgggag gccgaggcgg gcagatcatg |
| 14041 | aggttaggag attgagacca tcctggcttt gtgaaacccc gtctctacta aaaatacaaa |
| 14101 | aaattagccg ggcgtggtgg cgggcgcctg tagtcccagc tactcgagag gctgaggcag |
| 14161 | gggaatggcg tgaacccggg aggtggaggt tgcagtgagc cgagatcgca ccactgcact |
| 14221 | ctagcctggg cgacagagct agacttcttc tcaaaaaaaa aaaaaaaaag gaatctcttt |
| 14281 | ggttttatat atattttttt tatatatata atatatatta aaatataata tatatattta |
| 14341 | tataatataa tatataaata tattatatat tatatatttt atatattata tattatatat |
| 14401 | attatatatt atatatttat atatttatat attatatata tttatatatt atatatttat |
| 14461 | atatattata tatttatata taatatatat tatatattat atattatata ttatatatta |
| 14521 | tatatttata tatattatat attatatata ttatatatta tatatttata tattatatat |
| 14581 | ttatatatat tatatattat atattatata tttatatatt atatatttat atattatata |
| 14641 | tatttatata tattatatat tatatattat atatgtatat attatatatg ttatatatta |
| 14701 | tatatattta tatatataat atattgtata tattatatat ctaatatatt atatatatta |
| 14761 | tatatattat atattataat atatattata tattatatat tatatatatt tttatatata |
| 14821 | taatatgtat aatatataat atatataaaa acatatataa tatatattat atattatata |
| 14881 | tatattatat atattatata tattaaatat attttatata tattatatat atatacacat |
| 14941 | atatatatat aaatgaggcc aggctcggtg gctcacactt gtaatcccag cactgtggga |
| 15001 | ggatcacttg aagccaggag tctgagacta gcctgggcaa caaaacaaga tcctgtctct |
| 15061 | acaaaaggaa actgtaaaaa ttagctgggc atgatggcat gtgtctgtag ccctagctac |
| 15121 | ttgggaggcc gaagcaggag gatcgcttga gcccaggagt tcaaggctac agtgagctat |
| 15181 | gattgtccca tagcactcca gcctgggtaa cacagcaagg ccctgtctct aaactttttt |
| 15241 | tttttaattc tatttatatt tacatgtatt taaatgtgaa tattcactac ctatttgttg |
| 15301 | catgcctgca ttttttatac tgggcttgcc aaaaacccga acagctttct actttgacaa |
| 15361 | tgtatcagaa tttaaatcag caatatgtta ataagccaag caaaggttat atatgcaaat |
| 15421 | aaaactgttg tctataacct cctgttacac tggggcacag caaaagtcat ggtgtagtcg |
| 15481 | catgtgaacc tgtccctttc atagctgctc attgccagga aacatcagga atagccattt |
| 15541 | ggaagagtca tcagccctcc caccatccgt tttctgtctt gtcttttccc tatgagcagg |
| 15601 | ggaaattcca cgctggcccc aatccccagt gcagcggctc agcctctgcc tctgctgctg |
| 15661 | gtccccatga ggccagctta gaaacggagg attttgcaga acatccctaa atccgcttga |
| 15721 | ataatgaagt gatcattcat aaactcacct gaaccttatt aaaacctatt taatattttt |
| 15781 | cctggataat cctataggga taacttgcct cctgggcttc tctccaccgg gttcagttct |
| 15841 | tcctttagtg gtgaagttcc tcccttctta gcatctcaac tgtgcctgag aaaaggccag |
| 15901 | tggcggctgc actctgttcc ctgtggagtg ttaataaaga ctgaataaat tgaaataaat |
| 15961 | ccctttcaat gtcattaagt gctataaata atcatgaacc aatgttcgat ggctgatgag |
| 16021 | aaatgcaaga aaaaattttt aatcagtagg attcataagt tgacaatctg ggccaagtta |
| 16081 | aaaaaaataa aaataaaaag acttttaaaa agatcttatc gtttgttacc agtaagactg |
| 16141 | aattccagaa gcaagctact ccctcatttg tgggcccctg ttatcactgg ctgcttaggg |
| 16201 | ttgccaagcc ctgaattcat ttgtcaacta agagattttt ggccaagatt aagatttccc |
| 16261 | atgcctccat atttccatct gagaaatgga gattatactg tcttccccct cagaatggat |
| 16321 | gataatgtgg tctctcttct gttcgcatag tcatagaact gaaataaaac aacttaagag |
| 16381 | aattcctttg agcttctcag aagtgctgca gggctggggg atgcctccca ggagccgcag |
| 16441 | tcaggtgctg atctgaagtc tttggtgggc tgactttagc ctgacctgaa atagtatagc |
| 16501 | tgctgccacc tggctccctt agcgtcagtc agacggtgca gctggttcct aggggtgagg |
| 16561 | gctgagccag cagggtccgt gcccaggagg gatgcatggg tggccacagc ccagcctgca |
| 16621 | ctgatcttgt ctgtcccctt ctttggaagg aaggagcccc aaaccagggt gcaagacagt |
| 16681 | gggtgggggt gccttgagca tgacctcaag tgatttccag cccctgccag tgctgacttc |
| 16741 | tctggggaag ggctgggact tccttctggg ctcaagtcac gacccttgga tggaatttcc |
| 16801 | tgggagcttt tctgtttttt ctggagtttt cagttttttc ctaaccagac agggacttgg |
| 16861 | tacagaatct catattctaa ttatgcctag gagcagcctc tccccaccac tcacagtgtt |
| 16921 | tagcatgtga caggaatcga ttaaggcatg agtgattaaa ttaaagccag gcattgactt |
| 16981 | ggatggtgta atattctgac atctgtttgg tgtcaaaggc acggggcagg cgcgttaatt |
| 17041 | gaactgcttg cacctggcat ttgaattgag ccagagcggg gctaaagtca gtttgccttc |
| 17101 | accctgtaaa tggagggttt ctccggagcg tggatggtgg gaggtatttc agggtgtatg |
| 17161 | cataaccccc accctgacaa tggcccatct cttctccagc gtggccaggt ttgagtgcca |
| 17221 | gtcctgggtg tccagtggcc ccatagcctt gcgttttagt aaaatgctgc ccccattacc |
| 17281 | acctggtctg tgcacttcgg tcactggaat ttgccatctt ccagtcccga atgtggcaag |
| 17341 | ccatggagcc ttaagctctt ctccctccac atcctggaac agacccgcca gtttcttcca |
| 17401 | ggcattgcct cagtttgccc ctctgtttcc agtcacactc tcaccagcga taaaatgatt |
| 17461 | ttagacctta tcatctcacc ctcggatcct tatggaaaca ataatgagct gttccctgtt |
| 17521 | tcaattccaa aattcatatc caatccgttt tgcatgccat tgccaaattc ctcccagagc |
| 17581 | aaccccgtca cctgccctgg ccctctccaa gtgtggtcct gccatgggca tcgcctgcta |
| 17641 | agccaagctg gcctcgagct gcctgcccgg gtccccacac cttggctcac ctccctgccc |
| 17701 | agtcccgcct cctcccagcc tgccctgtgg ctccttcata gatgccgtgc tctttctgcc |
| 17761 | ccttgctcac ccatggcagc cttgcccctc tctccctgcc ccacccccta tttaaattga |
| 17821 | cctgaccttc ctcagtgtcc atcttccccg aagctttccc cagccttggc actcaaggtc |
| 17881 | cagaggctac gcgtttcctc tcacctgtgg cagcgccgtg ctccccagtg cctcacagtt |
| 17941 | tccttcttgc ccccgcttcc tgtgtaggac tcatctgccc acaggttgca cgtcctgtga |
| 18001 | gggcaaggac tgtgtcttat gtgactttcc ttctccagtc acagagctgg gcacatagat |
| 18061 | agctcaaaac cctctttatt aacacagttg gatgttgaga aatcaaacag gccaatgtca |
| 18121 | aatgagctct ccttatttaa atcaagtcag ttctccacct cctagcactc agttccagta |
| 18181 | ctctatatac atggaaataa taaaaaacac atttcctttg aaacattcta taatcgttcc |
| 18241 | tttgccctac ttcagaccaa cttaacgcac tccccattgg tccaaatgag ttttgctata |
| 18301 | cgaagatgct gataataata gcagcagtgg attattctgc taaaaccatt gcctcgttaa |
| 18361 | tcctcagtcc cgaggtgggg attattatcc tcattttgca gagaagcaaa ctgagactca |
| 18421 | gagatttcac agctggggag ggagccagct catccctctg tccaggccca agctctctcc |
| 18481 | cgcttgcctt cctgcctctg caacctcaga gcatccccca tctggttcta ctgcctgtgc |
| 18541 | tagtcgtgca ggagccaaaa gacacgtctt tagtgctaag gactggagaa gccatgccct |
| 18601 | ccagcctctg tgaatgggtc atatgtaaca tgagcctgga gaaattattt gaaaccaaag |
| 18661 | gcaagcctct aaaccaggct gctgcttcat ggcgccggtg acggcagaac caaatttagt |
| 18721 | gctgtgggca ggtccacact tatcaaatag agaagctcat ttttcttccg gctcacatca |
| 18781 | agcatgaaaa atgttcacac atacccccca cacacacatg ctttccggag gggtccatgt |
| 18841 | ggctagaggc tggaagatgt ggatgagagg agcctggcag gtaagcccag ggaagatgac |
| 18901 | attcagcttc ccagacagca tctacaggga gaaatttaat taaaagtggg gcggtttccc |
| 18961 | tgagcaaggc agacaaagtc agccctctac tgttaagaaa aagggtcaca gtgagagggg |
| 19021 | aggtgaggag actgagtctg tattttctag tctgttgggc tacactacct gatccccctt |
| 19081 | cctcaaaaat ccactttact ttccccatgt ctacaccaat gtggttcaca ctctgggacc |
| 19141 | aggaaaaggg ggagtgatgg ggaacagaga agggaggagc tcacacagct gaggctgggg |
| 19201 | ttatgcatat cgaattactt agaatttgca acctcacagg gtactttcat ggcgttgaaa |
| 19261 | tacacttccc acagccaccc tccctctaac taaaagcaag agtcatttct cagttctggt |
| 19321 | cttgcctccc acgttctcct ccacatttaa gaaaatccac cagctacaaa gtgaagatac |
| 19381 | catatgtgat atcccaccct agtttctgtt ttatcagggt ttggagcagg tggagcaggc |
| 19441 | agagggatca tttcagccta taaattgtat taagggtgag tactgagtca ttcttcaaga |
| 19501 | aaagttttag aagcatccaa aactgaaggg tggagccacc tggagacagt atcatcagtc |
| 19561 | ctggccccga gcatggcctg cataggcccc catggatccc agcgggagct gcagagtgcg |
| 19621 | ggcaccttgg cacacagccc tgagtgcaaa attaggagct gggcagaggg catctctctg |
| 19681 | tcgccattgg gcagcccagg gcacactggt catagcctta gaccacgaac accctgtgcc |
| 19741 | cgggggacag atgcaaccag tgtgccctgg gctgcccaat ggcaacagag agatcgacac |
| 19801 | ctggacccca tgtcacgggg actccactac taaggctcct aagactgcca ccttccagtg |
| 19861 | ggataagccc tgcctcctac tgggcccaca atgtgcagag aacacttggg actacctggc |
| 19921 | tttctggata cacaaatatt gatccaatct ggactaatta gaaggtcagt cccaataaca |
| 19981 | aatcgaagtc agctgggcgt gatggctcac tcctataatc ccagcacttt gggaggctga |
| 20041 | ggtgggcaga tcatttgaag ccagaagttc aagaccagcc tgggcaacat agcaaaaccc |
| 20101 | tgtctctact aaaaatacaa ataattaggc tgggtgtggt ggctcatgcc tgtaatccca |
| 20161 | acagtttggg aggctgaggc aggtggtcac ctgaggtcag gagtttgaga ccagcctggc |
| 20221 | caacagggtg aaaccccgtg tctactaaaa acataaaaat tagccaagca tgatggcatg |
| 20281 | tgcctataat cctggctact agggaggctg agacaggaga gaatcgcttg aatccaggag |
| 20341 | gtggttgcag tgagctgaga tggtgccact gcactccagc ctggttgaca gagcaagact |
| 20401 | ctgtctcaaa aaaaaaaaaa aaaaaaaaaa agccatgcct ggtggagcac tacgtgtaat |
| 20461 | ctcagctatt tgggaggctg aggcacgaga atcacttgaa cctgggaggc agtggttgca |
| 20521 | gtgagctgag atcgcgccac tgcactccag cctgggcgac agagtgagcg agactccatt |
| 20581 | tcaaaaaaat aataaatctg agtcacttta atattgttat ttggatgtca acctctaggt |
| 20641 | gtttgagaca ggagagtgat atgggggcac tggaaacaca caggcacggg gtgtcctcac |
| 20701 | acttgggtag cccacacgat gtgatttcag ggtgctggga ggtcccccca ctccccaaat |
| 20761 | tactaacaag tggatagtac tttacagttt atatgatctc atttgattct taacatgagc |
| 20821 | ctgtgagtga aaaattcctt cccctcttct acagattagg acgttgagat tcagggaggt |
| 20881 | tcagagggat tcagggaagt caagtggcac ctggagtccc gtggctaatt tgaggccggt |
| 20941 | aggggattcg aacccaggat ttgtgcttct tatgcctggg cttctgctcc ctggggcatg |
| 21001 | gtcttccccc tagctttccc attcactgct ttagcctagg ggtcctaccc tttattaaac |
| 21061 | tgccagtgcc tcactgcttt tctcccccaa agacaaaaaa aaagtgttct tgcttttgtt |
| 21121 | ttgtttttca tgggcagaga cctggaattt cagcttgaga atttgtgcca tatgataaat |
| 21181 | aaatcaacag atggcttttt ccttaaaaaa aaaaaaaaaa aaaactaaga tgtatttgca |
| 21241 | gtgaggcata atttgtacca aaaagtgctc accacactgt agtcatgggg gcaggaggca |
| 21301 | gccgcgggtg aagggagaaa tcttggagtc caggcagccc ccttctgggc tgaactgggg |
| 21361 | agctgggggt gctgccagcc ctgccaggtt ctcctaggag gcggcagctc atatggctgt |
| 21421 | gggaggaggc agagggagcc tcatatgcac ccacatttcc agggatctag aagacagaag |
| 21481 | gaggaaaacc accatcatgt taaagcagac agttaggtaa cacatcctgt aatacaagtt |
| 21541 | attttttcca catctaaagg ctaaaaatag ttgttagaat ttaaagataa ttggtaaatg |
| 21601 | agtttctatc cttctagttt cacatcaaat ggaatcatgc tgccttcaca tcactagtgc |
| 21661 | ccgttatttg tgtttaattt ccacaatgtt gtctaattcc actctttggg cttccccagg |
| 21721 | gatccagcct ccctcactcg cccatcgcag ggagatgctt tattcatctt tgtgtcttct |
| 21781 | gtgccgggca tagcgcatgg cacagaataa gcactcagta attgattcac gagtgaataa |
| 21841 | atggatgagt gggtgagttc aatattgact acaaaaaccc taaggccaca ctggtgagtg |
| 21901 | gctgcgcctg tagtcccagc tgctggggaa tctgaggcag gaggatctct tgagcccagg |
| 21961 | agtttgaaac tagcctgggc gatatagcga gaacctgtct caaatgacaa aaacagggcc |
| 22021 | aggtgcagtg gctcacgcct ggaatcccag cactttagga ggccaagatg ggaggatcac |
| 22081 | ttgaggccag gagtccgaga ccagcctggg caacataggg agaccctgtc tctacaaaaa |
| 22141 | attttttaaa aattagctgg gcatggcggt gtgcgcttgt agtcccagct actcaggagg |
| 22201 | ctgaggcagg aggatcactt gagcccagga aattgaggct gcagcgagcc atgatggcac |
| 22261 | cactgcactg cagcctgggc gtcagaacga gacctgctct caaaaaaaca aacaaacaac |
| 22321 | aaaaaaaaag gctttcttaa agagacttga gaacagaaag gggaacagat acataactta |
| 22381 | tatatttatt tgttcatctt tccaccttcc tggagggtgg aggggaacag gtctgtattt |
| 22441 | ggagttttga atgctaaaag tgggaataca tgtactgttt gccatgatct gttcaaaagt |
| 22501 | taagccaaat gccttagatt ctcctgaaaa ctggaatgcc actgtaaact ataagcccca |
| 22561 | cttcaaagat aaaagatctt gatgaacagg gctgggtctg tggactgggc ctctccccac |
| 22621 | cacacaagga agggtggtgc cagttgaagg aaaatcactt aaatccttgc tgtctcctaa |
| 22681 | taaggtgtgg tcccaggtag ggctgtcaga attagcaaat taaaacacag ggcatctgtg |
| 22741 | aaaattagaa tttcagataa caacaaataa ttggcatagg ctgcataatg tccctcaaag |
| 22801 | atatcaggtc ctaatctcca gaacctgtaa atgtgatctt atttggaaaa ggggtctttg |
| 22861 | tagatgtggt taaattaagg attttgagat ggggggatta tcctgtatta tctaggtagg |
| 22921 | tcctaaatgc agtcacactc atccttgtaa gaggaaggaa gagagagatg gaaaacacag |
| 22981 | aagagaagac aatgtggtga tggaggcaga gattggagtg aggtggccac aagccaagga |
| 23041 | ctgctggcag ctaccagcag ccagaaaagt ccaggaacca attctctctt ggagctccag |
| 23101 | agggagtgtg gccctgctga caccttagct tcaacctagt gatcctgatt ttggactttg |
| 23161 | gccttcagaa gtgtgaggga atgaatatct gtcgttttaa gccaccaagt ttatggtcat |
| 23221 | ttcctacagc agccacagga atcaaaaaca gtaagtatgt cccatgcaat gtttgtgaca |
| 23281 | cacaccaaaa atattacttg ttgttcacct gaaattcaaa tttaactggg tctcctgtat |
| 23341 | tttatttggc caacctagtt cccaggccca aagaaagagg cttttgaaat ttgcaagaaa |
| 23401 | gctggttgga gctgtcagaa agtggacttt gtaaacacag taccaccgaa ccaatttgaa |
| 23461 | ctgtactacc tctagacaaa agagagggca gtcagacagt tgttcgtgat ttcttctttc |
| 23521 | aacagtcatt tgagcactta ctacaaaaca gaagctatgt gtaagggtgg aggcgttagc |
| 23581 | tgttaatcag gacctccagg ctaagtttct gtattagtcc gttttcacgc tgctgataaa |
| 23641 | gacatacccg agactgggga atttacaaaa gaaagaggtt taattggact tacagttcca |
| 23701 | agtggctggg gaagcctcac aatcatggca gaaggcaagg aggagcaagc cacatcttac |
| 23761 | atggatggca gcagacagac agggagagag agcttgtgca ggggaactcc tctttttaaa |
| 23821 | accatcagat ctcgttagac ttattcacta tcaagagaac agcacagaaa agacctgccc |
| 23881 | ccatgattca gttacttccc accagatccc tcccacaaca tgtgggaatt caagatgaga |
| 23941 | tttgttacca tatcagttac caacccttcc agataaatca cgtgaaatat cgccattaac |
| 24001 | agagtgagct caggtggttc ttcagtgcat ttctgatacc tgaaccttcc ctgggaattt |
| 24061 | cacagaccat caggctctcc accctttgat agcaggatag cagggcccag gttctgcagg |
| 24121 | aggagatgtt accacaggcc tgaaagggag ggaggggcag atgctacagg aagatgctgg |
| 24181 | ctctggattc gctggaggag ctttcaaggg aagtagatac acactgtctc catcatttca |
| 24241 | tgtccatcac actctaaaat gctttggaca agaagcaaat gttaaagaca aatgtggccc |
| 24301 | attttcctgt acaaagaggg ctgctcccat gccaggctat tggcactggt gggcatgagg |
| 24361 | cttctctgct gccctggccg gggggttctc tcactcacca ttggctctct gacacctgga |
| 24421 | gagaccacca cccttgggct ttcatgatgc tcacagaatc cacactgttg gagctttaag |
| 24481 | gagcctggat caactggaac aggcagggag tactaggaca gcccagcatt gccccaaaat |
| 24541 | atccaggcct gataaaagag aaaaacaggt agctcacagg aaaaggataa aaaaaggagg |
| 24601 | agggatttaa catgaaaagg tgcttgatct ccctcataat aaaaagactg ctgattccat |
| 24661 | ccaggcaagt gacagaaaaa aaaaaattaa tttaaaaaga ctgctgataa aaccacagcg |
| 24721 | agacactgct gctcagggat ctgagggtgt gggcagccag gctgccacgc atcatgggtc |
| 24781 | ggagaggaag accacacccc tggagcagag ggcggctgat ctgtcagatg ccctttgaca |
| 24841 | gcacctcagc ttccaagaat taaccctttc tatgtgagca gaggcatcca tggggggaca |
| 24901 | cactggtgaa tcatctgtta tgtagaagtc tggaaaacat caggatggaa ctggtgaaat |
| 24961 | aagtgtggcc tctgacggaa tggagcggtc cgtctgcact gctgcgggtg cccctcagat |
| 25021 | cctgtgggtc agtgagaaaa gcagtgagga acaaggcagg tactgtgtac tgtcctctgc |
| 25081 | gtgcaaggaa ggccagcgca tgcaacagag tccacacaga catagcctaa ctctggaagg |
| 25141 | aagaatgaga atgcagtttc agtggtggcc tctggtgggg agaaactggg tgaagggaga |
| 25201 | tgtcatttcc atttctctac tattaatttt gtattaccat gcttaaatgt tactttttac |
| 25261 | cttttttttt ttttttgaga cagggtctct ctctgttgcc caggcaggag tgcagtggta |
| 25321 | caatcatggt tcactgcagc ctgaacctcc caggctcaag caatcctccc acctcagcct |
| 25381 | cctgagtagc tgggactata ggcacgcata ccaccgtgcc cagctatttt ttttaatcaa |
| 25441 | gatggagttt ttctatgttg cccaggctgg tctcaagctc ctggactcaa gcaatcctcc |
| 25501 | tgcctcagcc tcccaaaggg ctgagattaa aacgtgagtc accctgccca gccaattgct |
| 25561 | ttttaaaaaa gattaaatgc atgtatacgc tcaggcatca gcacacttgg aaaggatgaa |
| 25621 | aatatccgga agaagggttc ttttaaaagg ctcctcaagt gatgctggca ggcatgacga |
| 25681 | atgtccctgg tcacaaaagc tctgatctgg cctaaccctg tcatgttaga gactggagtg |
| 25741 | cgtgtgtgtg cgcgcaaagt gtggggggat gggggtgagt gtgtgtggtg tgtaagcatg |
| 25801 | agtgtgtatg tgtgtggtgt gggggtgtgt gctgtgtgag cgtgtgtgag tctgtgtgtg |
| 25861 | tagtgtgtgt gtgaagtatg tggtgtgtat gtgtgacgtg aggtgtgtgt ggtgtgtgag |
| 25921 | ttgtgtatgg tgtgtgcatg agcatgtgtg tgggcatgtg atgtgtgtgt ggtgtgtaag |
| 25981 | catgtgtgag tgtgtatgtt tgagcatgtg tggtgtgttg tgatatgtgt gtggtgtgtg |
| 26041 | agcatgtgtg tgtgatgtgt ctgtgtgtgg tgtgtgtgag catgtgtgct gtgtgtgtgg |
| 26101 | tgcatgtgtg tggcgtgtga gcgtgtgtgt gcattgtgtc tgtgagcatg tgtgagtgtg |
| 26161 | tgtgtgttca gcatatataa ggcatgtaac tgaacacagc actttagagg gctctcctgg |
| 26221 | agtcagaggg ggtgggtagg aggagaaggg aggtgggcta gtgtgctgaa gtatctactc |
| 26281 | cttgtcatag tctgtgacaa cccagactag cccatgagcc accctgttcc ctgcatttcc |
| 26341 | aatgagacct cggtggacat gttccctgag gtgaggctga ctgatgtcat ttgacgatct |
| 26401 | tgatgccaaa tccttttata tcaaaaacaa ccagaacact ctcttttctc ttagtgcttt |
| 26461 | cacccagatg accacatttc atcctcccag ccactctggg ccaggtggca ctgctggttt |
| 26521 | gaaagggagg tctcccctgg agtaacttcc gtgggcggat tcacaccctg cccacagtcc |
| 26581 | tgtcccagtc agcccaccat ggtggtctcc ggctcctcca gaattcccgc ttttcagctc |
| 26641 | atccccacat tcccggaggg actgagagcg cagccccagg gccctgctct ttgggggccg |
| 26701 | tctctacacc cagagaagca gcaaggcatt cctaggtttc tctttcagat gcagaacttc |
| 26761 | agtgttcaga gatgttccca ctggtcctga gagggctcag ttcagcttta atgactgcgc |
| 26821 | tgttgcgtgt gctctgcaga gggcgggtgg cccagcgtgg ctgactgcag ttttcctgac |
| 26881 | gtggagcccg agcctgcccc gctgtttatt aattaaggat cactctgctt gcagaaccct |
| 26941 | gaactcccca gaactgtgag gtgggagaac cccgagaggc cacctggccc cacttcccac |
| 27001 | ctgctgccca aaccccctct ctgccttcct gacagtcacc ccaactccca gtgatcccca |
| 27061 | tcaaccatct gacaagggga ctgagaggga agagaaagga ggggcccaaa gaggaaggta |
| 27121 | aaactgtcgg gaacagcccc caaatgtgtg acagccttca gtggagttgc ccactttccc |
| 27181 | ttttctcctc cctgcaggac ctcccttctc cccagtcctc cccaacttct gaggttacat |
| 27241 | tgagaaaagt ctgcagagag gtgccagcat cacaaggtgt taaggaccac gagtttggca |
| 27301 | ttttaacaga tgccagagcc acttgagaaa tgtggtaact aagcccagag aggtacagtt |
| 27361 | aacctcccca gagtcacaca gcaggttcat ggcaaagctg gactagcaca ggtgtccttc |
| 27421 | ccctgcagat ccccttctgt gccccacatc acctccctcc agtgtctggg ccacctggag |
| 27481 | atgggccctc agactcaccc ggccagaggt gccatctcat gggagaggtc tggccaggaa |
| 27541 | gcatcgatat ttgagatccc aagaaatgaa gacttggcct gtcagatgac agacttcggt |
| 27601 | catgggaaca cgtgatctgt tttacacatg cgtcccctca gcagcagctt tccagaacat |
| 27661 | tcccactttc ttctgtagtg agaagaactc tttccctgca gcctcctgcc caactcctcc |
| 27721 | ttcagtgtct ttgcttcagt gtctttgata aaccattctg ctttgcagag tgcgagctct |
| 27781 | gccttgcagg gttcgcatct gcctgtgctg agtaaccaac gctaaggtcg agtggtcggt |
| 27841 | cacctctcat aagagctagg gttgtctcat gctgatgact aggacttgcc ctcaaggaga |
| 27901 | aaaataaatc aaaacaaaag caaaaacagc aaacatgcat ctcttaaaga aggctctgag |
| 27961 | tccaggtaaa tttccttcca ctgaagcagc caggctgaat tcgaattatc tttgcccctg |
| 28021 | cttaaaaact aatgcaaatt ttcctagaga atatccacta attcctggag ggggcatggg |
| 28081 | cattcctgat gcccatgaga ggaccatttg ctcttccctc agtatgctaa ataacagaag |
| 28141 | cgacatttgt tgctggaaag tatcagtgaa gttaataagg tttttcttgc ccagggtgag |
| 28201 | ggaacagttc ccaatgacaa atgctgtatg ggaaggggct gtagaactgc cagccccttt |
| 28261 | ggtccatccg taaagtgaac tctgtggatc ctggaggatt ccagcgtctt tttttttttt |
| 28321 | tctttttttt taagacagag ccttgctgtc acccaggctg gagtgcagtg gcacgatctc |
| 28381 | agttcactgc aacctccgcc tcccgggttc aagcgattct catgtctcgg cctcccgagc |
| 28441 | agcaagacta caggtgcgca ccaccatgcc cgactaattt ttgtattact agtagagacg |
| 28501 | ggggtttcac tctgttggcc aggctggtct caaactcctg acctcaggtg atccacccgc |
| 28561 | ctcagcctcc caaagtgctg ggattacagg catgagccac catgcccagc cagcatcttt |
| 28621 | catttttctg tctgctttgg ccctttcctc tctcactgtc ttccttttcc atttccaaag |
| 28681 | tcagtccatc tcactattag cacaaaaact gctagagcgc ttgtcattgg tcatctctcc |
| 28741 | ctgcacctgg ctggtctgtt cttggccact gaagcgtttc ccccagctgt tgctttaatc |
| 28801 | attttattgt tattatgcct tacttaagaa atggatatga gatgcattta cctgtctctt |
| 28861 | cctgccactc ttcagagcca gtaagatgtg gtggaaaggg cccaggcttt ggaggagggc |
| 28921 | tggctggggt tggatcttgg ctgcccccta ctagctgtgt gaccttgggt aagtagctgg |
| 28981 | acctctctga gcctggttcg gaatcatagc acctctcttt cagggctgct gtaaggaata |
| 29041 | gcagtggtgt gtataaagca gagcgcacag ccagcaactg gcccctagcc acactgctga |
| 29101 | gcacctactg tgataagctg ccattgtggt gtgtgaagca aaggggaaac atgcctgctg |
| 29161 | tagtgagctt cctgtagggc aggttgtaga accagaggtg ggttccaagg ttacaaaggg |
| 29221 | actcttagtg tattagtctg ttctcacatt actataaaga cctacctgag actggatcat |
| 29281 | ttataaagaa aagaggttta attggctcac attggctggg tgcggtggct cacgcctgta |
| 29341 | atcccagcat tttgggaggc caaggccggc ggatcacttg aggtcaggaa tttgagacca |
| 29401 | gcctggccaa catggtgaaa ccctgtctct tctaaaataa aatacaaaaa ttagctggcc |
| 29461 | atggtggtgt gcgcctggaa tcccagctac tcaggaggct gaggtggaag aattgcttga |
| 29521 | gcccgggagg tggaggttgc agtgagccaa gatcgcccca ctgcactcta gcctgggcag |
| 29581 | cagactgaga ctctgtctca ataaaaaaaa aaaaaaagaa aagaaaaaga attgcaagaa |
| 29641 | ataaattatt gtttatgagc tatatggtct gtggtacctt gttgtgggac tgggagtctt |
| 29701 | ggcgtctccc tgaccctgcc tgttgctgca gcaccgctca gccctgcctg ctccctacct |
| 29761 | gcctcccctc ggcctctcct gcctccaccg ggcccctggt gcctcctcta gagacagtcc |
| 29821 | tcctgggacc gattgtgttc tcacttacac gaggcatcca ggactacaga taaccagagg |
| 29881 | aaggggcgcc ccccccgcct gccctcctcc ctggcatcct cacgctgcag aggtcagagc |
| 29941 | ctcatcccag ccccttacct gcccctactc tgtggagaac cgtggtcagt tcgccaggcc |
| 30001 | ggatccacga acggccttgt ggaagatggt gagctcacac ccagagctgg ctccgatgac |
| 30061 | cctgtctcct ttacatgttt ctaccttccc ctccctacct tcccccactg ctgggcgcag |
| 30121 | agtggaggca gatgaggttt aaagctcaga agggcttaaa cgggttgggg cgcagtggct |
| 30181 | catgcctgta atcccggcac tttgggaggc caaggcagag gatcacttga gcccaggagt |
| 30241 | tcgagaccaa cctgagcaac atagtgagac cgcgtctcta caaaaaataa aataaataaa |
| 30301 | attagctttg cagggtggca tgcacctgca gtccctgcta ctcagaaggc tgaggtggga |
| 30361 | ggatcgcttg tgcccaggag tttgaggctg cagtgagcta tgctggcacc acagcactcc |
| 30421 | agcctgagta acagaatgag atcctgtctc aaaacaaaca aacaaacaaa caaaagaagg |
| 30481 | cttaaagggg gctccaggtg ggcttggcag cacaaagcta tgaagttcta tcttagacac |
| 30541 | aagttctgtt actgggcctt tgcaggctgg cctgggtacc tggctgccat agacagggaa |
| 30601 | ccttccagat gagctgcagg cgtggagcac aggagccagg gtgctcttcc tgggctctgt |
| 30661 | ccacaggcag aacgtacaca gtctttgtac acgtccggcg gctctggtgc ctatttttgt |
| 30721 | ttgtgttttt cttttgtttg gggggatgga ttcggtttcc cccgagccct ctgtcctcct |
| 30781 | gtcacctggc tggtgctcgg caatgttgac cagctgcctg gctggagttg gcagtggcta |
| 30841 | aggctgtgac agctaacatg ttcctgagtc ctctcatttc ttcaccataa tgccctgttg |
| 30901 | agtttgcaga tactgtctct gtttttatct cccggggaaa ctgaggctca gagtggctag |
| 30961 | gccaccttcc catggtccct cagctcatga gggccacaca gggcattgcg gtggccttct |
| 31021 | cctcagcctt gaccctccgg ccccagcatt gctgcctcaa ggggtctcct ctgctgagcc |
| 31081 | gtgcaccttc tgcctggcag ctccaactct gtggctgtgt tcagtggctc agcactgccc |
| 31141 | cttgaccctc cctggccttc tgcggatgcc agactggagc actctgacaa ggtctggggt |
| 31201 | ggttgtatgg gtcctgtgac ctctatacac ctcccagtgc ctgggaatcc tgcagataca |
| 31261 | ccctccttag ccgtccctaa ccatagagga catttctgag gtccccgaga gagtggggca |
| 31321 | cccctgcagg atccaactgc tgggcccagg aaggatagca gcagcatgag gggttccatt |
| 31381 | agccacaaac tcacggcatg gaaccttcac ccacctcgcc cctcatctgc tgtttagcac |
| 31441 | ctggcacgcc gtgtatactt actgattatt acattttaat ggcaaattat agtggcaaac |
| 31501 | gtatgcatct ttgcacaatt gttgtacagc atgatgaaca agtcattaat agtaaagaat |
| 31561 | aaatgtgaaa gtgagaaaaa tctgactgcc aaagttttta ctccttcctt ccctccccag |
| 31621 | acttttaaat gaaagtttag ggataatccc ttagttgtcc tgctagtagg acttgcaatt |
| 31681 | aaaagaattg ggccaagaac acttctacgc ttctcctttt aggtttgggt gtaaattcgg |
| 31741 | ggtatttctc actgatgaaa gcctggtgca gggcagaccg tgggaagctt tcatttccgg |
| 31801 | aatggaccat caacatccct tggagaagaa ttctcttctc cagacccaga cctggtgtcc |
| 31861 | tggcacccat tgggcaagtg ggtcctagaa gacaaacctg gtcagagcct ggaggctgct |
| 31921 | tagcattccc cacgcacatt agcagctcgg agagctcagg aagccgcagc ccctccttgc |
| 31981 | ctcaccagcc tggatcagga cagcatcccc tggaagacac acagggcccg gcctctgatt |
| 32041 | acccagcctg gagggaaagc tcaatcgagc atcatgtcac ccggtgcccc catgcagggt |
| 32101 | ggcactggtg agacccccaa gccaatgata ccacctcaca ggagtgcagg cccattgtgg |
| 32161 | ccagatcatc ttgacttttc aagataaatc agaaatcgta tttccatgag atatccctat |
| 32221 | ttgcaagtga tggtgactaa attagaagtt tttgaatatt gtaacatgtt cgtaggctgt |
| 32281 | ttgtctggtt taaactctat ctggaggaat tcaagctaga cttcaggaat aacttcttga |
| 32341 | ggcaaggatt ttgagacctt agggaaagaa ggacgtcttg ggggtattct gactgttgtc |
| 32401 | ctcctggaag ggaagaacag agaactagaa gactgccctt agcgaagttc aaagcaccta |
| 32461 | agcccgggac cctcagcaag tgttcttgag tcacagattc tccctgaggc gcctctttct |
| 32521 | ggctccatag aatggctgat tctgtaactc ggtgagtttg cttttttttt ttcctccatc |
| 32581 | acccaggctg gagtgcagtg aagctggagt gccgtggagc gatcactgca acctctgtct |
| 32641 | cccaggttca agcaattctc cttcctcagc ctcccaagta gctgggatta caagcatgca |
| 32701 | gcaccacacc tggctaattt ttgtgttttt aatagagacg gcccgaagtg ctaggattac |
| 32761 | aggcatgagc caccgcggcc agccataact ctgtgactct tgttacaaag gccttatatt |
| 32821 | ttgctctttg agggtggttt tggtttgatg cctgttggtt gccatcttct aactagggat |
| 32881 | gttttatcaa aatgcccagc caaagtgtcc aaacaaatta taccttaaag tttgaaaatg |
| 32941 | tctggcactt ctaattcaat gcctgttgtg ccaggcactg ggctgctgag gaactgagtc |
| 33001 | ccgtccctgc aggctagcta gagaacacac acacacacac acacacacac acacacagag |
| 33061 | tggtcttaca agtcagtttt atattctacc tatatgcaat aaaggtatta ttatgttgag |
| 33121 | gtgccttgat ataaaaattt ttcttaaagg agaggatgcc taaaacaggc attacctgaa |
| 33181 | acctcctctc tccagcattg gttgtcttct gtcatgactc agggttttca ctgagaatgg |
| 33241 | gatggaaatg tggtctaaag atagggccaa tgctgggact ggatcccccc tgggaagtca |
| 33301 | gaccaggcta gggcaggtcc ttgaagccat caggaaaagc ctctggagcc agaaacaaaa |
| 33361 | caaaaaaaaa atggtgttaa ctaaactcag tctcaaatcc tgaataggac tcaagtcaag |
| 33421 | caaaataatt aaaggagtta gcaaagggca agtcagagag accgagcaac accaatgtct |
| 33481 | tccgggagcc ctgtggcgag tgacagagcc tggactctgg agtagaactc atcttgtgtc |
| 33541 | ttcttctgcc actcgttagc tgggtgacct tgagccaagc cccttaacct cttggaccct |
| 33601 | atgttcttat ctctaagtag gggctggtaa tatcttcccc tttgaggaat gccctctaag |
| 33661 | gggtgttgtg aagattcggt aaggtggcag gggtaggact cctggccaga aacaggcaca |
| 33721 | taataaatgc taagtctctc cttctctcca cctgctggat gctgtagata ctaaggattt |
| 33781 | cgatgtgaat gagacaaaac ccctgccttc caggagcctt tgagaatcag agaactagac |
| 33841 | ccatttccag aacaagggga tgcagggtct ggataaagtt ttggggatca atagagcaga |
| 33901 | gggctcccag aggatcccat agggttgact cctaactcaa gggcatgaga caacccccag |
| 33961 | gaagggcacc ctggaagggg tccggctgtc cctgatttac ttgtgggcac tgggggaatg |
| 34021 | cccggagcca tccagccctc agggctctgt gtgattctgg gttcctccca taaaagataa |
| 34081 | tcagattctt tcacgttaat gtctttctcc acctcattgc acatcatgca gctattcatt |
| 34141 | gactcagcaa gtatcagctt tgcatgcgac cttggcctac ccactttagc ttttagtaat |
| 34201 | agctcccttc ttgaataata caaccagtgg ggaaacagaa cctaactctt acctctggga |
| 34261 | ggcttatttg ctttgagaac atatgtcctg cagttttgtt catatggcag tgaagtttcg |
| 34321 | tgcacacact ctagagccag gcagcctggg ttcaaagcgc agctctgcca ggtcctaact |
| 34381 | gcatgaattt gggcaagtcg ctcaacctct ccatgcctga gtttcctcat ctgtaagatt |
| 34441 | ggagcaatgg taatacctgc tttttagggt tgagaagaga attaaatgaa ttaagatggg |
| 34501 | taaagtgctt agagtggagc tttgcaagta gtaagtgcta tgtaagtgct cgatttaaaa |
| 34561 | tgaaagaccc ttaaatacat tctttgttca tttcacaagc ccttcatttc acaaccttac |
| 34621 | atttcacaac caagctctgt ctcccctgga atccagccat aactctgctc acaagtgtga |
| 34681 | gacaggcccc agcagagctg cacgaagagg agagaaggca gccccccaga ctcccaaccc |
| 34741 | cctgtccaag atggcaaaac cagaacacag cctctgtacc accccagcag gtattcagaa |
| 34801 | tctgcaatct ccaaagccca cttcaattgt aaatgtagag ccacgtgcgc tttaagtcac |
| 34861 | ctgtcactct ggaggctctt ttgctcagtt cctcaccatt agcagggatg acagggagtg |
| 34921 | caggagtgcg gtcgactccc agatattgga gagcgctggg ctagctgccc attctcccgg |
| 34981 | cctccactcc tctttgctgt ccagccatca ctcgctcttt gaaggcaaac aaaacagaaa |
| 35041 | acagtgccaa aagtatggga agaaagccag cttctcccct ggggtgcctg tgatgccatg |
| 35101 | cccaccctcc ctgaccacgc agcccctgtg gaccctcagg gccccaagcc cccatttcca |
| 35161 | tcacatgcgt acacccatgt gtgtccatag ccgcccatct cagtcaataa ggctgctcct |
| 35221 | gcccacttgg aatagtggtg acaaccagga gtggcttatg ggaactatcc caatggcctg |
| 35281 | acagcatgtc cgctgcaaac cgctgaggta ggacactgcc ctcatgtcta gctgatcagc |
| 35341 | aagaggcgca gttgctttct taggtaacat tgctgctgtg tcctggccat tgctgggggg |
| 35401 | tggcacttaa tctacaccag aattttccct cctgtatctt ccaagctgct tggatcttgg |
| 35461 | tgctgaatta ggttggactt tgtcttgtgg ggaagggagg actatagacc ctcaacgtaa |
| 35521 | gcaatggtca gactattcta agaaaactcg ccgaattaaa gcatgaggta aatttagttc |
| 35581 | tgacttctgt ccaccccact gccactgtcc ccttttatcc catgatccct tgcttttctt |
| 35641 | ttcctcctct ctccctatct cttgtgtttg acgcatgata ggaattcaga aatatatgtt |
| 35701 | tgtggatttg tttattcacg tagcaaacca tttcttgagt gcctaccatg ggccaggtag |
| 35761 | aatgggcggc cccgggctgc agtggtttct tcagcccctc tccagggttt acactgtgca |
| 35821 | agacggtttg tgatgggtcc tcccatcgag gaccacactc ttctttctct gtgccccttg |
| 35881 | gtcctcagtc tctgacccca cttcaaaggc agcattcact cagggaagct cccatacaat |
| 35941 | gctagtcaga gtaaaagttt ggacaaattg ccaggaagca gcttgtcagt atgcataaac |
| 36001 | agcctttaaa atattactac tctttgaccc agaatttcac ttctaggaat ctgtcctaag |
| 36061 | gaagtagtca catgcaaaag atttatgtac caagatgttc atcaaagtgt tgttttataa |
| 36121 | caggaagtct cagaagctgg ataaatatcc aacctctgga aatggttaga tagaatagta |
| 36181 | tgtagccatt agaaaattat gtctatgggg tttaaaatgt catgggaaaa cacttctgac |
| 36241 | ataaaagagc atgagaactg tatatttagc ataatcttaa ctatgtttta gaatgcacag |
| 36301 | gaaaaaaatg tacaaacata ttcatagtga tgtctctggt ggtaggatta tgatcagtaa |
| 36361 | gtacttctgt ctcttcatat tttcctgtat ttgataatac atgcatatgt tgtttttaaa |
| 36421 | ataagaaaaa ttttaagttt aaaattggag ctgaaaagtg tttttaggtc aggcgaggtg |
| 36481 | gctcacacct gtaatagcac cactttggga ggctgaggca gtcagatcac ttgagcccag |
| 36541 | gagttcgaga ccagcctggc caacatggtg aaaccccatc tctactaaaa ataaaaaaat |
| 36601 | tagccatgtg tggtggcaca catctgtaat cccagctact tgggaggctg aggcatgaga |
| 36661 | attgcttgaa cccaggaggt ggaggttgca gtgagccaag atcgtgccac tgcactctag |
| 36721 | tctgggcaac agagtaagac tctatgtcaa agaaaaaaaa aaaagaaaag cctttttaaa |
| 36781 | cagtagcaga cataactata taatccttac taagctgtcg gtcaaatttt tatttatata |
| 36841 | tttattttat tcatttatta tttttagaca gggtctcact ctgttgccca ggctggagta |
| 36901 | cagtggcgtg atcatggctc tcttcaaact tgacctcccg ggctcaagtg atcctcccat |
| 36961 | cttagcctcc caagtagatg ggaccacagg tgcataccac cacacctggc taattttttt |
| 37021 | tattttttat ttttagagat ggtgtttact atgttgccca ggctagtctc aaactcctgg |
| 37081 | gctcaagcta tcctcccacc tcggcctccc gaagtgctgg ggttaccagc atgagccact |
| 37141 | gtacccagcc ctcaaatttt taaaaatcta taagagacat tattggacaa ttagagaaat |
| 37201 | tcacatatgg acttataata gtatcagagt gtgtggtgtg atggttctgg agggaatgga |
| 37261 | ctttttcttt ggagacaggc ttttctatgc ccaccctttt atcttgctaa cttatcatca |
| 37321 | tccaggttcc agcagaaaca ttacttcccc caggaaattt cttaagggtg cagtatcatg |
| 37381 | atgtctgcag caaattctca aatagctcag gaaaaaagta cgtgtgtggt atgagtgtgt |
| 37441 | gtatgtatgt gtgtatatat atacacatat atacacatat atatacatat atgtgtatat |
| 37501 | atatacatat atgtgtatat atatacacac acatacacat atatatacac acacacatac |
| 37561 | atacatgtat ttttatataa ttatatatgc agagagtgca aatgttgcca agttaaagat |
| 37621 | tggtgagtct aggtgaaggg aatatggtat ttattgtatt atttgtgcaa cttttcttaa |
| 37681 | gtttgaaaat tttcaaaaca aaaaattgga ggaagaaggc atgccagtct accccaagcc |
| 37741 | ctccattgga atgctgaaaa tctaaacaat gtgatttggc aatttcattt cttttctgtt |
| 37801 | gtgggccagt agtccttaga tgttggggaa gggggtagtc gctgaggtgt ggttgactta |
| 37861 | ggatggaaga agcagaagtc aagactccca gggtcaaagt ggtttgctct gctgacccaa |
| 37921 | gtgtgggagg cccagagtca gcgtttcagg tgtgctaatt cagcatggct ctattcacgg |
| 37981 | ccaaagtcca ccctgggcac ctctctggca gcaatcttgg gtgactctac taaggccagg |
| 38041 | cctccatgac cctatgtctg gatcccatat ctccacctct cccactgtct caggaacggt |
| 38101 | gcttagcttt ttcttttccc tctcctgtct tctttgccag catgtagaaa gtttaaataa |
| 38161 | ttcccctctt tacaacaaaa caaaacatac ccccttcagt caaccaccct agctctcttc |
| 38221 | tccttttccc agccagattt ttttaaaagc atcctaggcc aggcgcggtg actcacgcct |
| 38281 | gtaattccag cactttggga ggccaaggtg ggtggatcac aaggtcagga gatcgagacc |
| 38341 | atcctggcta acatggtgaa accccatctc tactaaaaat acaaaaaagt agccgggagt |
| 38401 | ggtggcaggt gcctgtagtc ccagctactc gggaggctga ggcaggagaa tggcgtgaac |
| 38461 | ctggtaggcg gaggttgcag tgagccgaga tggcgccact gcactccagc ctgggtgaca |
| 38521 | gagtgagact ccgtctcagg aaaaaaaaaa aaaaaaaaaa aaaaagcatc ctcagcactt |
| 38581 | tggcaactcc atctcctccc aacatgtccc tgttactgga atccagccag gactcagccc |
| 38641 | cgatctttct actctaacca gttgtctcag ttaacaagga caggtttatg ctgcagtgac |
| 38701 | aaacaagatc ccaaattctt gtggcttcac acatctggca ccacctcatc ttccagcctt |
| 38761 | aggagtcatc ttttagttcc ttgaaaactc tttacagttt tctgttgggg ccttgtcata |
| 38821 | tactattccc ctggaatgtt ctttcctatc ccctcccttt caccttgcta acttgtgccc |
| 38881 | atccttcagg tctcagcaga aacatcactt ccttggggaa gttttctcca acacccacac |
| 38941 | tacacaggtg tcccatctac actcctatga ctctgtggta cttgtctcac ttcattttcc |
| 39001 | actgccttcc ccacaaggca cctgcacaag ggcaaggacc gtaccactgt acctatgtca |
| 39061 | ctcattgctg tggtcacctg cactctggct gcctacctta actacacatt agaatcacct |
| 39121 | gaggagcttt taaagccaca atgcaagact ccaccctagg ccaattggat ccaaatccct |
| 39181 | ggggtagggc cagacatcag tggagttata tatacatata tatattttgt ttgtttgttt |
| 39241 | gtttgttttt tgagacagag ttttgctctg tcacccaggc tggagtgcag tggcgcgatc |
| 39301 | ttggctcact gcaagctccg cctctcgggt tcacaccatt ctcctgcccc agcctcctga |
| 39361 | gtggctggaa ctacaagtgc tcgccaccac gcccagctaa tttttttgtg tttttagtag |
| 39421 | agatggggtt tcaccgtgtt agccaggatg gtctcgatct cctgacctca tgatctgcct |
| 39481 | gcctcatcag cctcccagag tgctgggatt acaggcatga gccactgcac ccggccatca |
| 39541 | gtggatatat ttttaaagca ctgcagagaa ttctgttgca tcagcttgag aaccactgat |
| 39601 | ctgccttgtg cttcacattt aaaacttttt tttaatgaat aaataaaccc caaaaaatta |
| 39661 | atctccctaa gcctccctag aagataggat ggtaaggata ttttcctagg taaaaatatg |
| 39721 | ttaatttcat atttcatgaa atttcatgtt tcatttcaat caagctctgt catacacctt |
| 39781 | acatggggca agcccagtgc ctgggcaggg tgtaattata ctcattacac aggcaaggaa |
| 39841 | aagtcacatt aggtgatgga gcacaaatag gcagttaatg gtttcagggc tagttaggat |
| 39901 | atgtttgtct ttcaattgca agtaatagaa gcccaaagaa attggttatt tatataatat |
| 39961 | aattgattgg ttcccaaatt tgaaaaattc aggaatagac ccagcttagg tacagctgga |
| 40021 | tccagtcact caaacaatgt cacaaagaac cctttgacag gaatgtatcc tgtgttgact |
| 40081 | ctactttgct ctgagtagtc tttccccagg tgatgataaa aatggtcatc atcgccaggc |
| 40141 | ttgtgtcctg tttagtagga atatacaaga agagctcagt aaatgctggc cccaccacta |
| 40201 | agcaaaaaca aaacttttgt tgttgttatt gtcgttttaa ataacagctt agacctttct |
| 40261 | tctttccttg ttattctctt tcatctgtaa tccagttttc tacttctgaa gtatagaatg |
| 40321 | ttctgatgat ttattcttca ttacccacaa ctcgcacatg tttatttaaa aatgccagga |
| 40381 | ttgcctggcc gttgtgtgct gttaaccttt gtttgctgtt agtggatccc tgaagttcag |
| 40441 | gctcccaggg gagcagataa tgggtatcca gttcctgcaa tatccaccct ctggcaagcc |
| 40501 | aagttccttc ctgggtaagg ttttgcctac ctgcattcct agggaagttt ctgggcctga |
| 40561 | ccaccaagcc agctctgaga aggggtgcat aagccccacc atgctttggc tctgtcccta |
| 40621 | tagaatattt tatgttgtta ctgaaaacta aaggaagatg ggtgcggtgg ctcatgcctg |
| 40681 | taatcccagc actttgggag gccaagacag attgatcact cgatgccagg agttcaagac |
| 40741 | cagcctggcc aacatggtga aaccttgtct ctacaaaaac aaaacaaaac aaaaattagc |
| 40801 | cgggtatggt ggcatgcacc tgtggtacca gctactcaag aggctgaggc acaagaatct |
| 40861 | cttgaacctg ggaggtagag gttgcagtga gccgagatcg cactactgca ttccagcctg |
| 40921 | ggtgacagag caagattctg tctccaaaaa aaaaaaaaaa aagaaaagga aagctaaagg |
| 40981 | agagagacta aaatgatatc aggttcctgg agaacaaaca gacatgatct tgcttcatgg |
| 41041 | caggacagcc ggaagaagtg ggattatatc ctcacattac aaataagaaa actgagactc |
| 41101 | agaatggtta agtcacttgt cccaggccac acagccagta aattacagaa acagaatttg |
| 41161 | aacccaaatc ttccagctcc aaagcttgtg ttcttttcac tacctcctgc ttaatttttt |
| 41221 | aatttctaag attagaccct tcatctatcc atgacacctg cctgtcatcc cctgaaaaaa |
| 41281 | ggtgaacgcc gttcagaaat ttttctagcc tgagctcact cccagttcac ttatttttgc |
| 41341 | tttgtcatgg ctgcccagtc cccacttgta gaccaggaat aggtcatggc tgcggggact |
| 41401 | acacgctgtc gctgctgcaa gggccggcct ctgtttccgg ggctgagtgg gggccagacc |
| 41461 | tgccaggagc accatcttct gtgggtcctg cctggatgtc acatcccggc cccaagaagt |
| 41521 | cactgcaaac cttcgtatta ttgagcttca catcctagaa tttgctgtca ctgtggctgc |
| 41581 | tgcatgaagt tgtcctgaga gaaacgggca ttgtcattaa cagggaaatt gatggtctgg |
| 41641 | gggaaaagtc atcctcattc tcttgcagat ctatgggtga ttgagactgg ctgatgttga |
| 41701 | aggggtttct cagccatcgt gtgccatgtt atggaacagt ggtgtagcca gccatttgac |
| 41761 | acccagcgct gacctttgtt taacaacctc acctatatat gacaaaatga ttgtcagaaa |
| 41821 | taatcgtgta atgaaatgac tgtaataatg gccagaaaag aaacgcagat agtaaaatgt |
| 41881 | ttctcttgtt gaactctgta catataattg caccaggatt tttttcaaat aaaaagtaaa |
| 41941 | tattatacta caaaaaaggg aaaaagcaca agcatttatt aaatagcttt ctatatcttt |
| 42001 | ctgagttttg atcctttgat tgcagactga tgtaatattt tatgtaaatc attgcttggt |
| 42061 | tactaagtga actttaagaa aagtgagacg tctgcagaag ttgcccataa tttagcagct |
| 42121 | actgtattgt accattgatg tacggcttta ttttcttgat taattattta aacaatataa |
| 42181 | ttcacaattt taaaataata aatttccact taaaatggta tttaaactca gcaaaatata |
| 42241 | tcatctatga gtaaaatttg tatttaccaa gcaaaaatat tacagtttgt ggttcacatg |
| 42301 | ctgtctcact gttttaaatt ttaaatacaa aaactccaag taggctgggt gtggtggctc |
| 42361 | acacctgtaa tcccagtact ttgggaggct gaggcaggca tatcgcttga gttcaggagt |
| 42421 | tcaagatttg cctgggcaac atagtgagat cctgtctcta ctgaaaacaa ttagctgggt |
| 42481 | gtggtggcac atgcctgcgg tcccagctac tcaggaggct gagataggag gatcacttga |
| 42541 | accctggggg acagaggttg cagtgaggca agattgcacc actgcactcc agcctgggtg |
| 42601 | acagattgag accctgtctc aaaaaaagaa aaaaaaaaaa gaaacacaaa aactccaggt |
| 42661 | ggtcgcacag aatgacagga ctgaagtaac ttagctccaa tttctgtctt cataatcact |
| 42721 | gtcctaccat tgtctgtgct tagaatctac ttgcttaatg caggaacatg tgttctcaca |
| 42781 | gagatggaaa atgcaaatgg cgccagaagc aagctggaaa ttctgaacca ttaagaattt |
| 42841 | actctctgcc aggcacggtg gctcacgcct gtaatcccag gactttggga ggctgaggca |
| 42901 | ggcagatcat ctgaggtcag gagttcaaga ccagcctggc caacatggcg aaacttcatc |
| 42961 | tctacaaaaa tacaaaaatt agccaggcat gatggtgggt gcctgtaatc ccagctactc |
| 43021 | gggaggctga ggcaggagaa tcgcttgcac ctgagaggtg gaggttgcag tgagccgaga |
| 43081 | tctatctgca ccattgcact tcagcctggg agacagagta agactccatc tcaaaaaaaa |
| 43141 | aaaaaaaaaa aaagaactta ctctcaaaat aaatacgtgt ggctgactcc acatatggta |
| 43201 | gggccaactg tataactaga agttctccaa ataacttctg tggagaaaaa aaagtttatt |
| 43261 | aaaggttaac ttttttaaag tgctaactag aaccttacta acactgagat cgcaccaatt |
| 43321 | gtttataact tagacagggc cgggtgcagt ggctcatgcc tataatccca acactttggg |
| 43381 | aggccgaggc aggtggatca cttgatgtca ggagttcgag accagcctaa ccaacatgat |
| 43441 | gaaaccccat ctctactaaa aatacaaaaa ttagccaggc acggtggtac acgcctgtaa |
| 43501 | tcccagctac tggggagggt gaggcaggag aatctcttga acccaggagg cggagattgc |
| 43561 | agtgggccaa gatcgcacca ttgcactcta gccccagcaa caagagtgaa actctgtttc |
| 43621 | aaacaaacaa acaaaaaaaa aaacctcttg gaccaggaaa atatttttta agggaggagt |
| 43681 | attttatcac tggcattgtt taggattgca ggcacatgat gctaatgaaa agcagactaa |
| 43741 | ctattagttg gttttattac tgtttttgaa ctctctctct cccttttttt tttttttgag |
| 43801 | acagagtctc tctctctgtc acccaggctg gaatgcagtg actgcagtct cagctcacta |
| 43861 | catcctctgc ctcctcagtt caagtgattc tcgtgcctca gcctcccgag tagctgggat |
| 43921 | tacagggcac cacaccaggc taagtttttg tatttttagt agaggcaggg tttcaccatg |
| 43981 | ttgcccaggc tggtctcaaa ctcctggcct caagcgatct gcccatcttg acctcccaaa |
| 44041 | gtgttgggat tacaggcgtg agccaccgtg cctagccctg tttttgaact ctctagagac |
| 44101 | agtccagccc cttattactt gtcctgaggc agctgctccc ttcacctggc cccccgcatt |
| 44161 | gtgttccgga cccttgtcct ggtggtgcta aagaatatct ctgtcgatcc tttggggact |
| 44221 | ggggaaactg aggcccagtg ccacgcgatg ccatttgttc agggaagatt aggtcatctg |
| 44281 | ctaggtcccc agtcacttga ccttcttccc agacaggaag aagctgctct gggtctctca |
| 44341 | gtgctccacg tgtctttgca cattgaaatg ttctctgatt tttttttttt ttttttgctg |
| 44401 | ttacatttac ttttaaaaaa taacaagcaa taaaatgtta catttgagaa ggttgaaatg |
| 44461 | agaattgatt tgagttaaat tctagcagat ttttcttaga agaatgatat catcatctcc |
| 44521 | agctacctgc aattgatcta ctctgaatta agaaagagac ttccatttgt tgtttatatt |
| 44581 | ttgcactctt gatgtgtttc tttaaattat ggtcatgggc caggtgtagg agctcacacc |
| 44641 | tgtaatccca gcaccttggg actctgagga gggaggatca ctggaggcca ggagttcaag |
| 44701 | acctcgtctg tacagtaaat tttaaaaatt agccaggcat ggtagcattc acctgtagtc |
| 44761 | ttagctactt gggaggctga gatgggagga ttgcttgagc cagaactttg aggctacagt |
| 44821 | gagttatttt cacgccactg ccctctagcc tggctgacag agcaagacct gcctcaaaaa |
| 44881 | aataagtaaa aaataaatta aatttcaatc attagcagtc attaggatat ttaaatacag |
| 44941 | tatgttgaat caaagttacg catgtgtgta tttttttttc cagagagttg tttatcatgt |
| 45001 | gggttttaat ttaactttaa aaaaatgttg gctggacagt tgcccaaatg gtatcatcag |
| 45061 | ccatttggtt gagaacgtat gtcctgcggg ctcctctgtc actggagttt tgctagctga |
| 45121 | cagccactgg ctagttagag actgcagtca gcacagatgc aggcgtggac ttgcgcacgt |
| 45181 | aaccatgtca atgcaaagcc atcacttctt aaaaattctg aaccctgctg tctgagatgg |
| 45241 | tggtgcagcg gatagaactc tgctctaaga ggcagtagct aattccatgt cttctttgcc |
| 45301 | cttgactagc tgagtgactt tgcacatggg gcttgcctct ctgttgccct gtctgcaaag |
| 45361 | tggaatcatc ttttccttgc tagacagaag gtggaccctg gacctatggc ctttttgagt |
| 45421 | ttcccccccg cttcttagaa ggacctctga tcctactgag tttaataccc acgggttaat |
| 45481 | aattgggaaa agcaaaggaa gcgcttctgt ttaggtaatt atatgcatgt ttttgtcttt |
| 45541 | ttctggctgg aaagatatcc aagccactgg gaaggtccgt ggctacccag ggtagccctc |
| 45601 | tctggggagg gctgctatat ccaagagccc ctcatgagaa tttgaaaatc gaccatggta |
| 45661 | gggcctgctg acttttgaca gctaatggtg tgctgagaat tgtccctcca aagatgcctt |
| 45721 | tccattccct cgggagagtc tgggcagccc ctactggggg ctgggatgct ggctcttccc |
| 45781 | tcagcctcca ccccaactgc tctcttccct cctcccctcc ccagccccct aatttctctc |
| 45841 | acaaggcttt gttctgcagc aacctttcct aatgcagtcc tggcctcttc gcagcttcat |
| 45901 | tacataacct tccgtggact cctggtccaa ggatcacccc agaaagccag tcagaggtag |
| 45961 | gcacgcagct ggggtccatt tacttacctt ccccaccccc tcggaactca gaggtggtgc |
| 46021 | aggaatttgg actccaagaa ttaacagctc caccaccatc accagagcca aaactcagga |
| 46081 | tgcatgtgct tcatctgctg cttatttcca gctgagagcc agtggtgcca tggttcctta |
| 46141 | gggagccggt cccctgatgc cggctcctgg ccccaaatct ctctgatccg ggctcttcca |
| 46201 | gaatgtcttg tctccaccat cgcctttgac caatggtgtc cctttgcctg gtaatgtccc |
| 46261 | ctttgcctga tgatggccct gtcactcctc tctttagcac agaggaggct gtttcatccc |
| 46321 | ttcaagcctg ccctcccttc aagtcttagc tcaagttcac cttctccgca gagccttctc |
| 46381 | caatcttctt gactacgtct cctctcagct ccagcaacct ctgtctctgg cactgattcc |
| 46441 | ttacttagct aagagaatca cagacacttg gggctcagga caatctgctt tctctcttct |
| 46501 | tacccatggc cttggactgt gtgtacctct ttgtctccac tcccaaaccc aacccccaga |
| 46561 | gggcagagag catgttgtct gtccctttgc tcagcatgaa gccatgcgtg tggtagatcg |
| 46621 | gcagagttcc ataacttgtg ttgaccgagg ggtcactttg ctctgaaatt acccctgtgt |
| 46681 | ccttcagtat ttgcacagat agcttcctgg ccagaccgaa tatatccaag ggcatggccc |
| 46741 | acctctgctc ctgtttccag gtccctggtg ggggttagtt catgccttcc tcataatctg |
| 46801 | cccactggcc tggtcctcaa ggtcttccca actgctcagc cagagttgag aaaatgggtc |
| 46861 | gctccatcct gtttgtgtcg ttctctcctt cctggcccac tctcctgccc acaggtatcc |
| 46921 | aggggctgcc tgtagcatta gaggacatac atgcacatgc gtgggcatgg gacactcacg |
| 46981 | tagcctccaa gcacagcatc aataatgcat tctgtgcttt atagcatgga aagctgctct |
| 47041 | aaactttatt acacagtgga catgtctgaa gcagctccca aatccacccc tgagtgtgtt |
| 47101 | ggaattggca agcctatcac ttgggagtct agcttttttg ttcgttaata atagatgctt |
| 47161 | cctgtggccc cagcttggca attttgattt aaagtgatct taactgaaga gactaatgga |
| 47221 | cgggtctgaa tttgtgcctt ttaagcacaa agtattgctc ttaattaact ggattctatc |
| 47281 | ctttgagcag gcagaggcct tcccccaagg gcgtcattaa cgatccacat ctggacatct |
| 47341 | tccaaagcct tcttctgttt caggccaacc gcaggtgtgt tcctgaacac ccaggaggct |
| 47401 | atgagagcca catatgcctc ccaaatacac acagtgtgca tgcccaggga catagagcag |
| 47461 | tgtgcaaagt cccattccat ctctctccac ctgggagagg atggctcttc tgtctgattc |
| 47521 | atggctcaaa gtggtaaagg agctccccac tccccgtccc acgcctactc agagtctgca |
| 47581 | aatatgtatg cgatatgaga gctcgtcagt tagctgtctt cagtgtggcg cacatttgag |
| 47641 | gagtctgact cccctccagc acaggccaat gtgcactgct ctcctatctt tgtaccccca |
| 47701 | ctgttgcact gtgcagaggt tggagccata gaagtaccag agctgtgaaa ggagaggccc |
| 47761 | cctctcacct ctgccctggt ctccatcccc actttctcta ggaagctagt aggtgctgac |
| 47821 | aggggagaga agggagggga ggggtccaga aacagtggct catgcctgca atcctagcac |
| 47881 | tttgggaggc tgaggcagga ggatcatttg aggtcaggag tttgagacca gcctgggcaa |
| 47941 | tgtagcaaga ccctatctct acaaaaagaa aaaatgtaat tagctgggtg tggtggtggg |
| 48001 | cacctgtagt cctagctact tgggaggatg aggtgggagg attgcttgag cccaagagtt |
| 48061 | tgaggttaca gtaagctgtg attgcaccac tgcactccag cctgggcaac agagctgaga |
| 48121 | ccctatctca aaaaaagaaa aaaaaaaaga aaggagagag agagaaagaa aagaaaagaa |
| 48181 | aaaaaaaaaa gaagggaagg gaaagcccag aagagtgtgg ggagaggagg cggccgtcat |
| 48241 | tctggggccc tcagtgtgca caaccagata acacatgctc tgtgggcttt tgtaccattt |
| 48301 | tgcttgagca taaagaaagg aaggctgccc ctaaatagaa agcactctgg aggcaaacaa |
| 48361 | atctgactcc aatcctggcc ctgccacttt cccagctgag gacttagaca agcaccctag |
| 48421 | cctcttggac attctcagag ccatctgctg caagtgggtg ctgccatacc caccttactg |
| 48481 | ggcaggcttg ggggaccaag ggtggtaaat ggctcagtct ttcatgatgc ggccacacag |
| 48541 | caggtgcgcc atccaggtcc atttctttcc ttcctttccc ccaaatcaag ttgtcattaa |
| 48601 | agtactagtc cacattaatg aaatcaactg tattaatttt ctatttgctg ctataataaa |
| 48661 | tcatcagaaa tttagtggct taaaccaaca caaatgtatt accttacagt tctggaggcc |
| 48721 | agaagccctc cataggtgtc actgggctga aatcaaggtt ttggcaaggt tgcggtcctt |
| 48781 | tctggagggt ccaggggaga atccattttc ttcctttttc cagcttctaa aggtttcatg |
| 48841 | cattccttgg ctcatgatct tctatagcta tagtcagaaa aattttccat caatcatctt |
| 48901 | caaagccagc aatggcagga tgagtcctca catcaccttg ctctgacacc agttctctgc |
| 48961 | ctccctcttc cacatgtcag gaccctcatg attactttgg gctcactctg ataatctggg |
| 49021 | atgatctctc tattttagag tcagctgact gggaacctta attccatcta caaccccaat |
| 49081 | tcctctttgc catgtacagt gacatattca caggttctgg ggattaggac gagcctgtct |
| 49141 | ctgaaaggct actttacatg aaaattcatt tttttaatta agattttttt ttcctcttga |
| 49201 | gacaaggtct cactctatgg ttcaggctgg agtgcagtgg tatgatcaca gctcactgca |
| 49261 | gcctcgacgt ctctgggctc aggtgatcct cccacctcag cttccctagt agctggaact |
| 49321 | acaggggtga gcccccatgc ccagctaatt tttttttttt tttttttttg agacagagtc |
| 49381 | tcactcagtc acccaggctg gtgtgcagtg gtgcaatctc agctcacagc aacctccgcc |
| 49441 | tcctgggttc aagtgattct tgtgcctcag ccccccaagg agctgggact acaggtgtgc |
| 49501 | accaccacgc ccgactaatt tttgtatttt tagtaaagat ggggtttcac catgttggcc |
| 49561 | aggctggtct caaactcctg atctcaagtg atccaccaac ctcagcctct caaagtgctg |
| 49621 | ggattacagg tgtaagccaa catgcccggc cccagctaat ttttaaatat tttttttgta |
| 49681 | gagatggggt tttaccattt tgtctaggct ggtcttgaac tcctgggctc aagcaaacct |
| 49741 | cccaccttgg tctcccaaag tgctgggatt acagcatgag ccactgcact cggccttaag |
| 49801 | agaagattta ataattaata ctttacaaca agatctggaa gaggtgggat gagtaactaa |
| 49861 | atgaggatac aagtaacccg ggtcatattt gctaataccc ttggtcacat tgaacttgat |
| 49921 | atcttatcag attttcctaa tcagctcctt tagcagcagt gttgcagcat cttatctcat |
| 49981 | tttgtttttt gtttttttgc ctagcacatg cctgtaaatc actggattga ggtgtttaga |
| 50041 | tgtttgttgt cctttggatg cttcttataa atccatattt catggctccc tggaaagtgc |
| 50101 | tatgcaaatg ataagctgca aggatggaaa ggaaattgca gtgctcctga attgtaaatg |
| 50161 | ggcttttacg aggaggtttc taattactcg ctctttctct tgaactgagg agttgaagtg |
| 50221 | taggtggcag atccataaca gataatcatg tgtgtgatgt gacttcagcc tgagcgtcga |
| 50281 | ggaccaagtc acagagcagg aacagccact ctccagtgtc cttggggcta cgtctgagga |
| 50341 | gaacctggga tttcatatat gacctgcact ggctgggggg ctctcttgac gtaacgtgtt |
| 50401 | ccctctgagc atgttacaga ttctgacatt cttatgttcc ttctgtggag agacatgtac |
| 50461 | ttagtgacct aactcacttt agcatatttt tgctcatcgt ttgtgtagct taaaggaatc |
| 50521 | agataattac cccctcccca ctactttcgg aagcacaaat gcaatgccct agaattgtac |
| 50581 | tggggactca aaaagaaaag agagtagtaa aatctattaa aggggacaaa gacagcctat |
| 50641 | atactacaag ctttctattt ttatggcaga gaatgccatt ttctaagtaa acagagaact |
| 50701 | gcatttgacc tgcaatatca aatgcatgga tttgatgctt tggaaagcaa ctgttttctg |
| 50761 | cgttaatctg ggtgtcttcc gtgaaatgtc ctcctgcctt tggcttaaac actagctttg |
| 50821 | tctacagcca ttccatcctg aacctgccca atcttgtctg aatcctggct tcaccactga |
| 50881 | caagctgtgt gtccttgggc aagttacttc acctgtctgt gcttcagagt cctcatctgt |
| 50941 | gagttgggga atctggacag aatctacccc atagggcgta gtgaggatgt gttgaattat |
| 51001 | cccaagtggc tacacagagt aagcactcaa atgatgtcat cgttgtcatg attgctgtta |
| 51061 | ccagagccta gagttcattc tgatactcga gtctgtggcc catccagccc aggtaaggaa |
| 51121 | tagttggagg agttgggcat gttcagcttg aagaggagac gacaggggat atgggatagt |
| 51181 | tgaatctgtg aagggccccc tgggatgaag aactggcatg ttctgtgtgg ctccagggca |
| 51241 | ctgagcagga cccatttgcc aaagtctcag ggacacagtt tctagctata gacagaaaaa |
| 51301 | ttttctgtca ctcagaggat gaaaatagaa tgagccccct taagaggtaa tgagctccct |
| 51361 | gtcattggaa ggattccaga agagctaggt aaccacttta ggtgctatca aggggctttt |
| 51421 | ttctttaaag tcctttccaa aagcttctga gattgcataa acaataggaa gccatcttgg |
| 51481 | tgctttaaca caaactctcc ccagtgatga gggttgagcc aaagccagat tggcaagcag |
| 51541 | agaggagact tgtgtacaag gagttcctcg agtcaattgc tttttccttg ttctagccag |
| 51601 | ccagagggct cctgttggaa aacaggagac cggagaggct gaggcctgac caaaccagct |
| 51661 | tctgcaggcc agctgggagg ccacaactcc tacctacggg aaaactgaag ggcatctcta |
| 51721 | tttttagatt agcaaaagaa aataaattta agtttgagtc tcctttgcaa cttttaaaag |
| 51781 | acatctttat tgagatgatc attcacattc tataaaattc ccccactttg agttacaatt |
| 51841 | cagtggtttt agtcttcctt gatgattttg atggtctttt cttaaggctc ttggaagacc |
| 51901 | cagaagcctc tcagacacag gtgggtgtgg agggcgtagc acagaggcag acttctcatt |
| 51961 | tcctgggtct cccctttaat gactctcaga gacccctcct tccccctgcc cctggcttct |
| 52021 | accccagggg tgtagagttt tgccattttc caagcagaac ttcatttcct cttctgtgtc |
| 52081 | tacactcttt gtgcttcttt cttgccagct ttctctcctt tgcccgccct tccttccttc |
| 52141 | cttccctccc tccccccttc cctccttccc tctttccctc cttcccccct tccacccttc |
| 52201 | cccccttccc cccttccctc cttccttcct tccttcctgc ctgccttcct tccttccttc |
| 52261 | cttccttcct tccttccttc cttccttcct tcctggtatg tgactaattt ctgtttcagg |
| 52321 | acataaatgt tgtccaggct gttctttggt ctctctgttg gataatggac atttggcatt |
| 52381 | gagagaggct gctttttctg aaatcatgtt cttggggccc agaacctagg tgtgtgcttc |
| 52441 | tgactttgtt ttcttcctga tccaaattct gatatgtcca tttaaattga tctagaccca |
| 52501 | cagggcactg tgggacagat cctcagtgga acatgactct gtaacgagag cattttgttt |
| 52561 | tgtcaaaatg agaacatatt attgcctttc atctgattgt aaacataata catgtttata |
| 52621 | aaacagtata atgagacaaa aatgtagaca ctaataaggg aaaatctccc taattgtatt |
| 52681 | tctcttcaca gagaaagccc ctgttgggca tatatactct agtttgttta tttgtttgac |
| 52741 | tacacatata tgtattcttt tcttatgtat aaaaattctg aacatgcaca tttctgcaac |
| 52801 | tactgttttc acttgatgat gcatggacct ctctagagtg tacgtttctt cttccttaca |
| 52861 | aagcagttgg cttcgcccag ggtgcaccag gacacggttt tggctctgtc cccagggtgt |
| 52921 | cacgggacca ggggatgatc tcacagggtc tgccatctgc cctgcctggc cggaggctgc |
| 52981 | atcgagaggg ccaaggggca ccacgtgtcg tgggtactgt caaacaagag ccttcagagc |
| 53041 | cttccacagt ctttcttttg cttcccagca ttgcttcccc gctggtggac tctgaatcta |
| 53101 | gaactagctc caggcgcctc tccaaattca gacgggagct ggggcactat tataatgcaa |
| 53161 | atctaggcaa agccctccca ataccaggat ccagaatggg gtggggccct ttgccctgaa |
| 53221 | aagctgttta gtttgaaaat acaaacagga gacagaaaag tttggctaaa ttaatggata |
| 53281 | aagttttaac gatggtaacc atagtagggt tcatcgacag ccagcgatgg ttctgaacac |
| 53341 | ttgacatgta ttaactcacc taatccccac attttacaga caatgcaaag gaggctctgg |
| 53401 | gaggttgagt gacttgcccc aaagtcgcac agctcctaag tgaaggattc ggagtggact |
| 53461 | ccaggcagcc tggtctgact ccctgcactg cgctgtgctt atctctggcc ccaatgccgc |
| 53521 | catgcagaag tgtctggggg cactttgtct ctgtcagaca gaattcggag atgtgtatgc |
| 53581 | ttgccctggt atggcacttc tctttttttg agacagaatc tcactctgtc accctggctg |
| 53641 | gagtgcagtg gcatgatctc agctcactgc aacctccgcc tcccaggttc aagcaattct |
| 53701 | tgtgcctcag cctcccaagt agctgggatt atagatgtgc accatcgtgc ctagctaaat |
| 53761 | ttttgtactt ttagtaaaga tgttgttttg ctgtgttggc caagctgatc tcgaactttt |
| 53821 | ggcctcaagt gatctgccta cctcagcctc ccaaagtgct gggattacag gcatgagcca |
| 53881 | ccatgcctgg cagtgtggca cttcttacgt gtgttcagcg gacactgttt atcttctgtc |
| 53941 | cctccaagac ggtgctgagc tcaggtcgtt cattactggc agacaactgc tgatttccaa |
| 54001 | cagaattgcc atcctcttct cccctgcgac tttcagagtg tgacctcaga ctcaaaaatt |
| 54061 | agaagtgaaa acatcttaaa aactatcacc ttttcttcct aatcctcctc tcccctccct |
| 54121 | gtcttccttg ttgtccccat ctaatgaact atcatggcaa aaagagccca tttctggtca |
| 54181 | ttttctgtgg cctttcaaac tcccacctac cccactgctc ctgggtgcat tacccgaaag |
| 54241 | ctgagacttc agtgcagaaa gtgccaggcc ctctgtcccc ccagatcgcc ttccttgtct |
| 54301 | tccctgtgct tgcctgtcac attgtgtggg ttccagcgct ggaaggaatg aggaacagat |
| 54361 | tctctggttc tccttttgaa gtttaccttc gctccaccac ttctgagacc ttcccggaag |
| 54421 | ttgccccttg tttctctcct ctccagggct gccccagagc tgcctctcac ctcttcctgc |
| 54481 | tgtcacccca ccaccatcag ggcagaagtt gggacaaagc ctctcctact ggctcctgct |
| 54541 | tttctccctt aggtccagcc tcctcttctc catcttcagg agtctccttc tccactcaca |
| 54601 | cgtcatgact tcagcacctc gcatcagtcc agaatatgac tgcttgttca agtgccacct |
| 54661 | ttctcatgca tttttttcta gtgacaatca cagccaccct gtggggcagg agtgtcatca |
| 54721 | tccccatgtt tcaaatgaag aattgcagtt cagagagggc aagtgactgg cccagcctca |
| 54781 | acagctagcc agtggacccc accagggctt ctgactccag tccgggttcc ctttccaccc |
| 54841 | aaatccatgg agggagctga gccgagaaca ggtgtccttc aggaagacgt gaagccaaag |
| 54901 | cctccacctc caaactcagg ggcccaggga gtccaggcac ccatccactc acaaggctgg |
| 54961 | atatggtgca ttccaggaga ggggttgggg gcgagtggcc tctctgtgta cccgtgggga |
| 55021 | tagatgcgca agtggcatcg ccacatcgtg agtcctggct tcatgggtga gctccaggtc |
| 55081 | caacgagaag ccaagcaggg ggcccttcaa gctcagcttt gggcccgggt cggggtacag |
| 55141 | ggtagagcgg gcctccccag cccctgccat gaggccaagg cagtgcatcg ttcgcagcgt |
| 55201 | acattcagaa accaaagcct aggagctggt tatcattccg gtttacagct gatggaagag |
| 55261 | caggtgcttc cgagaaccca cagtgctctt tggccagtga cccaagggtg cctctgagag |
| 55321 | gcctcgcagc acccggaggt gctgctgagg caacgccctg actgtaagaa ggaccattca |
| 55381 | tcctcagaga gtggccgtga tgctgctgcg acagtcccac catccccctc gactctcact |
| 55441 | cccaacagac ttcccactgt aaagctgaac tctccagcaa atcacctctc gccagactct |
| 55501 | ctcctcactc tctctgggtc cactagaggt tcctcagcct ctctttgcct tggttttccc |
| 55561 | agctgtaaaa tggagcaaag agggcctatg tacccacaaa ggtgtggttg gagcgactcc |
| 55621 | tcctacatta gggcctcgag tggggcttca tgattggttg gtggaggtct ccaaacccac |
| 55681 | ccagtgccac cgaaggctga gactgcagat gcaatgccac aggtgtccct cctcagcctg |
| 55741 | ggcagctgaa catcatgtgt aaaacgggga taataagata ataacagccc cttgcaccta |
| 55801 | tgtggctgtg aggattaaac aagataaatg tgtaacagtg cctggctata gaaatattta |
| 55861 | ctcttgttat taagggaaga atatgtgtgg ctaaaaaggg atcgaagatg taaaagccaa |
| 55921 | tccctccccc tctagcatat ttaagggtaa tgctgagttg gtttgtggac catttgctgc |
| 55981 | ctgttagagc tggaaggtag ggaccccctc tcaacagcga tgctacaaat tatacccatt |
| 56041 | ggaggtcaac caaaagacaa agcttattgg ctggacatgg tggctcacac ctgtaatcct |
| 56101 | agcactttgg gaggccaagg caggcggatc acttgagatc aggagttcga gaccagcctg |
| 56161 | gccaacatgg tgaaacccca tccctactaa aaatacaaaa attagctggg cgtggtggtg |
| 56221 | cacacctgta atcccagcta ctcaggaggc tgaggcagga gaatcactag aacccaggag |
| 56281 | gtgaaggttg cagtgagccg agatcgcacc actgtactca aaccgaggca acagagggag |
| 56341 | acgcaatctc aaaaaaaaag aaaaaaagac aaagcttgtt aataccagca tattgttaag |
| 56401 | ggaataaagt aggctgcaga acaactggtg taatatggtg ccatgtaggg aaaattacat |
| 56461 | gtgtgcatag gagaggggtc tgcaaggttg tgccctaaga tgttagagtg gttcctttgc |
| 56521 | ttttctcttt tataattttg tatttgactt ttaaataagg accataaatc acttttataa |
| 56581 | aatacattct ctccagcccc tactactcct ttaaagaata agagtggttt gcccaagaaa |
| 56641 | gacagttttt tttgctctgg ttttcttgat tctgacatca gaggaaactg cttctcatcc |
| 56701 | acttggggct ctgggttcag gggattcatt tcaggcagat taaagtggtg accaggggca |
| 56761 | ttcgtggaca cagggaggga caggagcacc atcagtttgt ctcacacaac cactgtcatc |
| 56821 | ctcactgaag gctgttgcct gatcaaaaac agtattgggc caggcacggt ggctcacacc |
| 56881 | tgtaatacca ccactttggg aggctgaggt gagtggatca cttgaggtca ggagttcgag |
| 56941 | atcaacctgg ccaacatggt gaaaccttgt ctctactaaa agttcaaaaa ttagccaggc |
| 57001 | gtggtgggtg cctgtagtcc cagctacttg ggaggctgag gcaggagaat tgcttgaacc |
| 57061 | cgagaggtag aggttgcagt gagccgagat ggcaccacca cactccagcc tgggcgaccg |
| 57121 | agggggactc tgtcttaaaa aaaaaaaaaa aaaatatata tatatatata tatatatgtc |
| 57181 | aaaaatgggg tagtttttag atctatagta gttctaaaaa caaaggccat ccaagcatga |
| 57241 | cagatttaca agcactattg gctattccag tagttacaat ggaggagaga agcttttagt |
| 57301 | taaaacaaac aaacaacaca acaaacccag aaaccttagg tcaaaaccaa aattgtcctc |
| 57361 | tcagacacaa tctgggaatt ttctcatgac agtgggcatt agccaactga catcagcagc |
| 57421 | aaccatccgt gtgcacacag tggcaccacc tcctcccaaa aagcagcctt catctatgcc |
| 57481 | ctcatacaat cgttgattat tctctttgga ttgaggcccg gaattattta agtttcttct |
| 57541 | tgccagcatg agtctttcct ttctgtatgc tccttatctt ctctctttaa tttggcagtt |
| 57601 | ctgcttgaaa tctgggtctt tcattagtag tagttcaatt tggttccaga acattctgtg |
| 57661 | gtgtgatgca atgtgaccag agctcacact tcagagctct tcaagggcca gtcttactga |
| 57721 | gcacctccca gtggctgcct gtgtgctggg cgccacttgt ggtgggcagg agagaggagg |
| 57781 | ggacacaaaa ggagacacag ctccttctta gaagctcaaa gttggggacc agctgccaca |
| 57841 | gaagagtatg tttagcatct gagacaccaa gatccagcgt cacaagggtg tttattaagc |
| 57901 | ctcctcatct ctttcttttt cttttttttt ttttttttcc tcaggcagtc ttactctgtc |
| 57961 | acccaggctg gagtgcagtg gcatgatctc ggctcactgc atgcaaccac cacctcccgg |
| 58021 | gtttaagcaa ttctcctgcc tcagcctccc cagtagctgg gattacaggt gcccaccacc |
| 58081 | acacccagct aatttttgtg tttttagtag agacagggtt tcaccatgct ggtcaggctg |
| 58141 | gtctcgaact cctgacctca gatgattcac ccacctcggc ctcccagtgt gctgggatta |
| 58201 | caggtgtgag ccaccgcgcc tggccttgct gttgattcat ctatagtatg tttgacttga |
| 58261 | tgacctccag ttaccttaga cagaggttct catctaagct ccaactttcc atttcctttg |
| 58321 | tcctcgtctt tccccttaac ccctccacat ttctctcaaa atcaccccac ttctaaaaaa |
| 58381 | tactgtttat ttttctttta aatttcaaat tatctatact cattgaaata aatcaaaata |
| 58441 | gcatggaata agcgaaaaaa atggatccca cccttcccca ctcccattcc ctagggctaa |
| 58501 | ccatagttaa ccatttaatg actaggtttt ttcgttgttg ttatttttta tttatttatt |
| 58561 | ttgagacaga gtcttactct gtcacccagg ctggagtgca gtggtgtgat ctcggctcac |
| 58621 | tgcaacctct gcctcccagg ttcaagcatt ctcctgcctc tgcctcctga gtagctggga |
| 58681 | ttacaggtgc ctgccaccac acctggctaa ttcttgtact tttggtagag acagggtttc |
| 58741 | tcaatgttag ccaggctggt ctcgaactcc tggcctcaag tgatctgccc accttggcct |
| 58801 | tccaaaatac tgggattaag gtatgagcca ccgcacccag ccctcctggg ctcttttcct |
| 58861 | ttagttgcac tcgctccccg ctcctggagt agagggattt ccgagagact gtgggctcca |
| 58921 | gccttcacct aggcccagga ctaggatgcc tgccctaaca tttatcttta taccttaaag |
| 58981 | caaaacagct ggaccataag cattcaagaa caaactgtga ataaggagaa agttctccca |
| 59041 | ggaaacaaga gctttagttc tgttgggcca gcccttatat tccttagctg ttaccagtca |
| 59101 | ctgcttgatt taatctcggc tatcacttgg cctgacaggt ctgctgctgg tgccaggatg |
| 59161 | tctgggtttt gaagcctggc tccattacat acttcctgtg tgaccttggg caacttactc |
| 59221 | aacctgtctg ttcctcagtt tccccagctg tattatgtca gcataatagt ttgttgtgtg |
| 59281 | aattaaatga ggtaataact ggaaatgctt caaacatggt tcctatcatg agaaatcctg |
| 59341 | ctttccgcct aaatgtgctg gaaaattcct ggtggtgcag aacaggagac cagagcaaag |
| 59401 | gaaagacagg gtgcagaagc caaaaattac ctcggagaac aaagcgcatg ttaaggttat |
| 59461 | ttttggattc taggtttatc tctgcttggt cttcagttac ctgcaagaga tccatttagg |
| 59521 | ggatttttgt ttgtttttaa cgatagcttt attgagatat aattcatatg ccataaaagt |
| 59581 | cactctttta aaatgtttcc ggtatattca caaggctgtg cagccttccc tgtccttgat |
| 59641 | tccagtctga gtttttaact gaagggataa ggaggaccac gctttcccca gaccagaacc |
| 59701 | gcgggccagg gggcgattct gctgagtcac cgcgggcgcc tggtgcgcgg cggcggagcc |
| 59761 | cgggaccttc cttggctgcc ccctagcgag ggccgcagcg cagcctgaga cacccgccgg |
| 59821 | ggccgctcca cggccgtcgg atttagactg gaagctcggt ccaggtcccc agcttgatgc |
| 59881 | gcccgcggtg taggagacca gcccgactcg ggcttcccct gagcccctgg actcttgact |
| 59941 | ccagcagggc ctgggtaatg aacgtcagct cccctttccc aaaggggttg ctctgttggg |
| 60001 | aaggcacccg tttgatacag tagcatagag atgggtttta gcatcaaaat atcagaattc |
| 60061 | aagccttgct ctctgcttac tagctgtgtg accctaaaaa ggtttctgaa cgtctctgag |
| 60121 | cttcagtttc ctcatcattc cttctcacgg ggtggttgtg agcattacag agatcctctc |
| 60181 | tgtgaagccc ctgtgagtgg ctcatcctga gggctgaaat aaacatgtta ttaataatcc |
| 60241 | aaaactggca agggatgttg actggtcccc ctcccttgcc caaggagctt tctagaacct |
| 60301 | gagttatcat taccaaactg tactgccttg agtaagaaag ttagaaggaa tgggaaggat |
| 60361 | ggtggcaggt ggaggaaggc ggattggtca tcacctcctt gcagcaagaa acagccccag |
| 60421 | atcgtgggaa acctacagac ctgctagaca gactaggagc aaaagctggg gctttaagaa |
| 60481 | tccccaggga ggttctcctg agagagtagc cagttggatt ttgtaagcag agatttgttt |
| 60541 | ggggaggagg tgacaacgta gggagcagag gggcaaagct gtcgggaatc ctgccttgag |
| 60601 | ggcagggatg tgtgttgggg ggagttgggt cactggggct cggtggcctt gggcaagttt |
| 60661 | ctacctctca ggtcctttac ccacctaggg tcgccatcct gcccacctca caggttacag |
| 60721 | tgagcctgga tgcactgtca tgggcaggtg cccaggaaaa tggcagacat gttccaaaca |
| 60781 | gcacgcagca ttccccagtg atgcccaggg tcaccttgga ggtgggcgag atgcctgggg |
| 60841 | tttctcgtcc accccacaac acctcagggg acagccaaag ctgtcccttc aggtaagctg |
| 60901 | cacagaagat gtgaactctg ctgcaaagac tcvattcttt gggagcaaaa gggacccagg |
| 60961 | gtctcacctg cacatccctg tccctgaggg cctaggggtt cttggaggcc ccagccttgg |
| 61021 | caaaatgagg aagaaggtga aggttgtctg ggcccctgcc aggctccttc ctcggccacg |
| 61081 | cactcccctt cctgcacaca cacccttctc cctccacccc atctccatcg ttgtcagaaa |
| 61141 | agtcacaata aaaaggtcca tattgtctag ttcccatact tttaattttt aaaattttat |
| 61201 | ttatttattt atttatgtat tttttgagac agagtcttaa cccaggctgg agttcagtgg |
| 61261 | catgatctag gctcactgca acctctccct cctgggttca agtgattctc atgcctcagc |
| 61321 | ctcccgagta gctgagatta cagatatgtg ccactatgcc cagctaattt ttgtattttt |
| 61381 | agtagagacg gggtttcacc atgttggcca ggctggtctc gaactcctgg cctcaagtga |
| 61441 | tctgcctgcc tgagcctccg gaagtgctgg gatttcaggt gtgagccacc gcactcggct |
| 61501 | ccacactttt cacttattaa aagactgtgg tgtccatcaa tggatgaatg aataaaccaa |
| 61561 | tgtggactat ccctcccatt acccaaggaa tgaagcacgg agccgtgcca agatctggat |
| 61621 | tcacagtgaa agaagccagt caccaaaagc cacgtgctgt gtgacttccc ttatacgaaa |
| 61681 | tatccagaag agatacatcc atggtgacag aaagtagatg agcagctggg gactggcgaa |
| 61741 | ggggagaagg gggagcagct gtctatgagg tccagccttt cttctgggct tggtgagaat |
| 61801 | gttttggaac tagatagagg tgatagttgt acaacattgt gaatgtacta aatgccactg |
| 61861 | aatcattcat tttaaatcgt tctttacgtt gcatgaattt taagtcaatc aaaaacagtt |
| 61921 | gtttgaaaag agaaaagcct atgggtagcg gcagcagtga ttggatttat gattcgattc |
| 61981 | catggctcat ccctcccctg cctcaccccc tcgccctccg acgtcttctt cttttactct |
| 62041 | gaactgttat ctttgttctc atctctctct ctctctctca accctgcaga cacttttccc |
| 62101 | tttctttgtc tgcccccacc ctccagattt ccgtgtctcc agtgtctccc tacgaggcat |
| 62161 | gaattgagac tgggagggtg tgattctgaa gaaggcacca acagtgactc agctagcccc |
| 62221 | ttcccccacc ccgccccccg ggcctcaatt tagctaaaaa accacaggga cggactcagg |
| 62281 | aggcaatacc tttccaaggg tccctaaaaa atgtcccatt ttagtgtcca ggtttcactc |
| 62341 | aactttagtg cctcccctaa aatgtgttcc ttacctccca ccccactgca tctaagtcac |
| 62401 | tgcctgagaa aacaggattg aggaaaggag aaaggaagag agagagagag gaggagagag |
| 62461 | agagagaggg aggaaggctg atggatttag aaaagaagaa aacaagtggt ctgaggaaaa |
| 62521 | cagccttggt gtgtttattt tcctgtctgt gtatcgcttc tcggccttct ggctaagatc |
| 62581 | aagtgtattt tcctgtctgt gtgtctcgct tagattacag ggatctgtgg gtgatgacac |
| 62641 | gtctggtcca ggctgcgtag tcacctcaag ggcatgctta ttgatgtgtt tttcaattca |
| 62701 | ctatctttgc atgggagtcc caggccaaga ggcacagctg cgccatttgt ctgttggttt |
| 62761 | agatatcctt tatccagttc ttccagagaa atcatcctgc ccttctggag gaggtgggca |
| 62821 | gcaggggtca gagatgggag ggaaaggaag gagccaggtc cttggctagg atgccagggt |
| 62881 | cccctgcctc tcacctggcc tgggctggag gcctcctgct gtcctgtcac tgatcactac |
| 62941 | cccgccccag cctcctgagt tagaagacac aggctaaagt agagtatttc ttcattgaaa |
| 63001 | aacccataca aaataaaggt tcataaaaaa taaaaattta gactgggtgc tgtggctcac |
| 63061 | acctgtgatc ccagcacttt gggaggccaa ggcaggtgga tcgcttgagc cctggggttc |
| 63121 | atgaccagcc tgggcaacat agtgaaaccc catctctaca aaaaatacaa aaaattagcc |
| 63181 | aggcatggtg gtgcatacct gtggtcccag cttctcagcc tatggaccca catagaatac |
| 63241 | aatgtcagca taagaaggga gccctggggt caccaaatgg tttgggcggc aaagaacctg |
| 63301 | aaggttgaga gaagtggctt ggttacccag ctgttggatg tgagacctgg ccactgcttc |
| 63361 | ttccataccc tagacctgca ccctgacatc tcaagtaaaa agttggggga tgttttatgg |
| 63421 | tccaggatga aggaagggca gtgaggggca gcggagcatc actttgcatt tctgtctgcc |
| 63481 | tcttactggc tgtgtgacct ggggcaggta acttcccaga ctcctgggaa tcataacacc |
| 63541 | tatgatgatg atgatgatga tgatgatgat gacacctacc tcaaggattg ccctgaaggg |
| 63601 | tcacagagat gcctgcaagg cacctgcatg gagcaagcgc cccttctctg gcaggtgctg |
| 63661 | ggtgagcact acctgctgcc aggccctggg gctatggcac tgcgtgaccc tgcaagtcct |
| 63721 | acctggcgaa gctgtcgttc ttgtgctcag tcagtgttgg ttgtaagact gagaagagtc |
| 63781 | acttcatttt gctctccagg gacatctttc tgggtcctat tttctgccta tgtcaagtag |
| 63841 | cgcctcaagg atgctcctga aaatgggctt gtctttctta acatggcagg taggtcccaa |
| 63901 | agcattagca tggggcagct gacctagccc agccaatgca gtgcagtgac tcttgcaacc |
| 63961 | gagtctaatc agaaggtcca tgaacctacg agcatttcct gtcccaggat cagggtggag |
| 64021 | gctgagcctc cctgcttaga gattcttccc atgcattcca cttttttccc caaaagaaaa |
| 64081 | tattgaccct tgagaggcac acagtttatt tattttgcat agtaaatagt agcctgtatt |
| 64141 | ttaaggatga gttgatttct gcatcagccc ctgtaggtca tcagccttct attggtgcat |
| 64201 | ctgactctct ctagccctgc agggatggtg gagggggagg ggaaggaggg atctttattg |
| 64261 | gaaaccagga cagtgagact cattgccctg tcatctgctc tgtggtgctg aatgaggcag |
| 64321 | cccaacagag aaataccctg agcgagcatc cccagcctcc aaaacagtgg cgcattgccc |
| 64381 | tgagtcctgg gaatgacctt tgattctcct gctcctgact tggaacccat ggaaacctct |
| 64441 | agaagcagct gaggaaaacc caacatgaaa agcagaactc cacactgaga atataggagg |
| 64501 | tgatcggaac atacaatgat tcttgctaag accgattcac agtttttctt ttttttcgat |
| 64561 | cgaagaaata ctggagaagc ctaaagaagg agtctaaaaa ctctggcacg tgggccaaaa |
| 64621 | ctgtccttga gctaagaatg attttcacat ttttaagtgg ttgaaaaatg aaataaaata |
| 64681 | agatgatgtt ttgtgacaca tgaaagctat gggaaattca aattctaata tctataaata |
| 64741 | gtgttttatc agaacacagt catgctcatt tatttatgct cgatggctgc tttcccgcta |
| 64801 | caattacgtt gagcagttac aacagagacc acgtggccca caaagcctta caatatttac |
| 64861 | tatctggccc tttccagaaa aaaatgtgcc gactcttgac cttaacctca gcaatttggg |
| 64921 | aggccgaggc aggcggatcg cttgagctct ggagttcatg accagcctgg gcaacatagt |
| 64981 | aagactccat ctctacaaaa aatacaaaac attagccagg catggtggtg cacacctgtg |
| 65041 | gtcctagcca ctcgggagac tgaggtggga ggatcgcctg agcccaggaa gtcgaggctg |
| 65101 | cagtgagctg tgatggcacc actgcacctc agcctgggcg acagagcaag accttgtctc |
| 65161 | caaataaata aataatgcaa agtaaaataa ataaaaccat ataaaaagga atcaatttaa |
| 65221 | aattataatg aaagctggcc gggcatggtg gctcacgcct gtaatcccag cactttggga |
| 65281 | ggctgaggtg ggtggatcac gaggccagga gatcgagacc atcttggcta acacggtgaa |
| 65341 | accccgtctc tactaaaaat acaaaaaaaa aattagccgg gcacagtggc gggcgcctgt |
| 65401 | agtcccagct actcgggagg ctgaggcagg agaatgtctt gaacccggga ggtggagctt |
| 65461 | gcagtgagcc gagatcgtgc cacttgcagt ccagcctggg cgaaagagcg agactccgtc |
| 65521 | tcaaaaacaa aaacaaaaac aaaaacaaaa aaaaattata atgaaagcca aggggcatag |
| 65581 | tagaacaaat tttctagagc tcattaagtc aaatgagtca ccagttagta aaacgcagtc |
| 65641 | acggggaaga gagggcagga ttctttgaag cagcggctct cctaaaaaca acccaccctt |
| 65701 | gtccagctgc cttccctcct gagggtgttc cctttgactg tgtgaccccc atcccctatt |
| 65761 | tcccaaccgt ccaagcccac ctctagcata atacgagctt ttaatccctc tccctgaccc |
| 65821 | caacccgatt ttgaagccca gtctagtatt ttctcaaata cacttcttgg ctccattcct |
| 65881 | tcctttccat cacctctgcc ttttcactgc atgcttggac cactgcagtc agctccctat |
| 65941 | gaacagttgc tctctaccca tccaatcggc cccgcctgct gctgccaaat tcaccgaggg |
| 66001 | cacctctgtg gtgctgcctg tggacaaagt ccaagccagc cacctcaccc acctacaggt |
| 66061 | gagtggggag cagccagcgt gtccagtggt ttaccccatc gccacagact tggtgatgtg |
| 66121 | tcgatgtgca gagaaggggt gttggcagcc acaacacaag caaccccgcc ccatgtgaga |
| 66181 | tctaagatgg gcgtgctggg agccacctct gagaatccaa cagaaggcag aggggagaac |
| 66241 | ggctcacacg gcacaaacac tccttccttt tttttttttc tttttccttt ttgaaaggag |
| 66301 | tctcactcta ttgcccaggc aggagtgcag tggtgcaatc tcagctcact gcaacctccg |
| 66361 | cctcctaggt tcaagcgatt ctccagcctc agcttcccaa gtagctggga ttacaggtac |
| 66421 | actccaccat gcccggctaa tttttgtgtt tttagtagag acggggtttc cctatgttgg |
| 66481 | ccaggctggt cttgagctcc tgacctcagg tgatctgcct gccttggcct cccaaagtgc |
| 66541 | tgggattaca ggtgtgagcc atggggccta gcctccttcc atttaaatgt atgcctaatt |
| 66601 | tgcccattga gaacggctga gacgcatttt aagtggccag ggtctactta gagttagtgc |
| 66661 | tcatgaccag gcccaggtca agcctggctg gccagatggt gcctttgacc tgctctgtct |
| 66721 | ctgtgcaaag gaatgagctg aaggatgggg gtgcagtgtg tgggcagtgg gctggggctg |
| 66781 | gcaggactca gtgactaagg gaagagaact ttcctcacta ccagcctgtc ttttcagggc |
| 66841 | accgcggggg gctttgggac ttggtgatga acacagcaca gagagctgtc cagcatgcgg |
| 66901 | gtccctggct tctcacactt cccaggctcc ttcagaggct ctctccaaag ggagctgctc |
| 66961 | tctctagaac ccatgaattt ggaatatagg caaccactgc attggggacc actgacctca |
| 67021 | aacatagaga ccagagcaaa tggggctcat cacgtgaaac tcatctggaa ctctagcagg |
| 67081 | ttcttttata tatatatata tatatatata tatatatata tatatatata tatatatatt |
| 67141 | ttttattatt atactttaag ttctagggta catgtgcaca acatgcaggt ttgttacata |
| 67201 | tgtatacatg tgccatgttg gtgtgctgca cccattaatt catcatttac attaggtata |
| 67261 | tctcctaatg ctatccctcc ccactccccc caccccacaa caggccccag tgtgtgatgt |
| 67321 | tccccttcct gtgtccaagt gttctcattg ttcaattccc acctacgagt gagaacatgc |
| 67381 | tgtgtttggt ttttttgtcc ttgcgatagt ttgctgagaa tgatggtttc cagcttcatc |
| 67441 | catgtcccta caaaggacat gaactcatca ttttttatgg ctgcatagta ttccatggtg |
| 67501 | tatatgtgcc acattttctt aatccagtct atcattgttg gacatttggg ttggttccaa |
| 67561 | gtctttgcta ttgtgaatag tgccgcaata aacatacgtg tgcatgtgtc tttataacag |
| 67621 | catgatttat attcctttgg ttatataccc agtaatgaga tggctgggtc aaatggtatt |
| 67681 | tctagttcta gatccctgag gaatcgccac actgtcttcc acaatggttg aactagttta |
| 67741 | cagtcctacc aacagtgtaa aagtgttcct atttctccac atcctctcca gcagctgttg |
| 67801 | tttcctgact ttttaatgat cgccattcta actggtgtga gatgttatct catggtggtt |
| 67861 | ttgatttgca tttctctgat ggccagtgat gatgagcatt ttttcacgtg tctgttggcg |
| 67921 | aactctagca gcttcttttc acaagttcat ggagagaggt ttcccactga gggaatcaca |
| 67981 | tctgtctgat caaaagaggc ttgggaaatg gctctcctgt tcattccctg aaaacctctg |
| 68041 | atggaaccac tgccactgtg gcagccccag cactggcacc ccagccatga ttggtgcccc |
| 68101 | agccacatct ctgctgtgag ccccagagcc ctggttaatt aatcatccac gtgttgatgg |
| 68161 | ggagaggccc attcacaaaa gcgacataaa gcccagggag acgtggccgt ggcaagaagg |
| 68221 | gtgtgggact acattccgcc cccaactgag agattcagaa accagaaaaa aatggaaaaa |
| 68281 | catactgtgc tcttgggtgg gaaaactaaa tatcatgaag ggagcaattt ttatagtttt |
| 68341 | ggcctataat acaattccag ccgaaatccc agtggaactt tgagaatttg caggaaaaaa |
| 68401 | aaaaatgtct aaagtacatc tggaagacaa acttacaaga aggtcaaata attttgaaaa |
| 68461 | agaaaatgat atctaagccc acctagagaa taagacttga gatccaaagc taaatcagga |
| 68521 | ggctctagca aaattgacag ataagcagga cagagtgcat ggtgcattca cctggggaag |
| 68581 | agggcagatt ggtctacaaa taggcctggg tccactgact ttagctgtta tatttgggga |
| 68641 | gaaacttttc aacctcactc catcttaaac ctaaaaatat tccagatgaa ttaataaata |
| 68701 | taaaaaatta gaccactaaa aatgtagaag aaaatggatg atctttctat accatagagc |
| 68761 | aatggaataa atcacaaagg aaaacagatt tgactatata aaacttaaac cctgcccatc |
| 68821 | aaaaaccatc agaaaccaaa ataaaaggca accaactgga gaagatagct gccacaaata |
| 68881 | tgatcaaggg ttaatgttat tcataaatta agagcccaca caagtcatta gaataagcac |
| 68941 | tgagacctga acagacaagc aaaaagaatg agagtgggtc ggcgcggcgg ctcatgcctg |
| 69001 | taatcccagc actttggaag gctgaagcag gcggatcact tgatcccagg agttccaaca |
| 69061 | ccagcctgag caacatggtg aaaccctgcc tctacaaaag tcataaatat tagccgggtg |
| 69121 | tgatggcaca cgcctgtagt cccagctact caggaggctg aggtgggtgg atcacttgag |
| 69181 | cccgggaggt agagtctgca gtgagccaag atcacaccgc tgcactccag ctggagcaac |
| 69241 | agagtgagac cctgacttaa aagaaaaaaa aaaaaaaaga ggagaaaaat gctgatctca |
| 69301 | ctagtaatta aaacatcagg ccaggcgcag tggctcacac ctttaatccc agcactctgg |
| 69361 | gaggctgagg caggcagatc acttgagatc aggagttcta gaccagcttg gccaacatgg |
| 69421 | tgaaatcccg tctctacaaa aaatacaaaa attcgccaag cgtggtggca catgcctgtg |
| 69481 | atcccagcta ctcgggaggc tgagacagga gaattgcttg aacacgggag gcagaggttg |
| 69541 | cagtaagctg agatcgtacc attccagtcc agcctgggct acagagcgag actctgtccc |
| 69601 | agaaaaaatt aaaacatcac atatttaaac aactctagga tatcatttaa aaaaacatta |
| 69661 | atagactgtt ttttagagca cttttaggtt cacagtgaaa ctgagtggaa ggtacagaga |
| 69721 | cttcccgtat gttccctgcc ctccacgtac agcctccccc actgccaacg tcctgcacca |
| 69781 | gagtggtaca cttgttacaa ccaatgaatc ctcattaaca tatcattatc acccaagttc |
| 69841 | atagtttaca ttagtaaaac atcatctttc atctataagc acaaaaattt tttggcattt |
| 69901 | atttaggtgt atgattaact cagtgttgac aagactcaca cttcataccc acttgcactg |
| 69961 | catctgagaa gcaattggtg tctacagccg ctacaccctc aacaagcccg atcttgtttg |
| 70021 | aaaagcaatt ggtgatgctt ctcaaaattc tatggacaaa gtcagccggg catggtggct |
| 70081 | catgcctgta atccctaaac tttgggaggc cgaggcaggc agatcacctg aggtctggtg |
| 70141 | aaaccctgtc tctactaaaa atgcaaaaat tacccaggca tggtggctgg ggcctgtaat |
| 70201 | cccagctact cgggaggctg aggcaggaga atcgcttgaa gcaaggaggc ggaggtttca |
| 70261 | gtgagccaag attgcaccac tgcactccag cccgggtgac aagagtgaaa ctccatctaa |
| 70321 | aaaaaaaaaa ttatggacaa agtttttcaa aaagatattt aatgcaacct tatttgtaat |
| 70381 | attggaacat ctgaggccat ttcagtgcta actattaggg gatggttagg aaaatatggt |
| 70441 | acatatgtgg aaaggaacat ttggtagtta gtgcccctga tgtttacaaa ggcttttagt |
| 70501 | gaccaacaaa tgctcatgct ataatcttat gtgaaaaaag caagtagcat aattgcaact |
| 70561 | atatttttaa tgcatagaat aaaaggctag aaggaaatat cacagatcct tgacatacat |
| 70621 | tcccaaacct ttgtaaatcc gcggattcat gaaaacagac acatttgcac aagtgcctga |
| 70681 | tcttttctgt tatacattca ttagaagtca agccctggtg ccacaaagta tctgcctttt |
| 70741 | caaatgtgat cagaatgttc tcttttgctt caaggccatt tttcacgaag cagtggcatt |
| 70801 | tttgcctctt catcagagtc accgtgtgcc ctggaggact gagaacagca gagccgtttt |
| 70861 | aggatgggac agggcagcca ggaggattgg gctcactccc tactgagtgc ctcactcccg |
| 70921 | tacagccccc atagaggaag aggggttcaa atttattcct cagccagatg gcatgtgccg |
| 70981 | cctgtcctgg aatttcacat cacttatgat ggaccaaaat tccaaaagct gaatccatga |
| 71041 | ttgtcaaagt ctggtatggc aggatgtcaa cagtaatcgt ttctgggcag agggatgatt |
| 71101 | ttctcttccc atcttgcttt gtataaatac attttctata ataaggttgt attacttttc |
| 71161 | tcatcaagaa atagcaaagt actgttttac tcaaaatatg aatagagcca ggcatggtgg |
| 71221 | cagcttatgc ctgtaatccc aacactttga gaggcggata tgggaggatc actttagccc |
| 71281 | aggagtttga gaccagcctg ggcaacatag tgagaccccc gtccccactc ccccaaagaa |
| 71341 | aacccacaaa gcatttatcc tggattattc acaggggcca aaaaaaaaaa aaaaaattca |
| 71401 | ggcctcctat agccatgagc tacgaatatg aaaatatgca aatgtgtaag aaaagccagc |
| 71461 | acatccgatt tttactttta ctttcacacc tctgtccacc atgttccaag agaagaaact |
| 71521 | tggtcattga aaggaataga tcaaatccaa agaacaaaac cactgtgctc attaaacttc |
| 71581 | ttagtgttca caaagcttta gctgcaggtt gaatggggca acccgaattg gctggctcac |
| 71641 | ctgggctgca gggagcagag atcgcgacac tgcactccag cctgggcaac aaagcgagac |
| 71701 | tctatctcaa aaaaaaaaaa gttcataaat tcaaagttat gaattatttt taaaataata |
| 71761 | ataatttaca ataaagatga ggacaaagtg tgagtaaatg gtggtttcta tccagctctg |
| 71821 | ttgagctgaa gtggcatctc cctgctgggg ctcttgggga agaagggtgt gtgttgctct |
| 71881 | tcagatccca agcctcatgc ccctactggg ccctgtgggg tgcttctcag cccaccagga |
| 71941 | gagccaccgt tggaacacac acgtggggga cctggtgggt gccggtgtgg tgaatggggg |
| 72001 | ccacagcctg actccaggaa gccagcaaac tcggagctgg aggagtcagg acacccccga |
| 72061 | tgagtcaaga gttggttttg ctgccagttg acatctgatt gaaccatctc ttcacttctc |
| 72121 | cgtgcctcac tttccttacc agacaggctc tgctgatgct gtccctctcc tgttcagtcg |
| 72181 | tgccctcacc gttaaagaga aagagcaaac tgctgggcag cagcattgat ttttttaatg |
| 72241 | aagtggaaag agagctggga ataacaagtc gggcccacct cacctgcccc acctggtggg |
| 72301 | tttatttgtt ttgttttttt ttttttgttt tgagacagag tttcaccctg tcacccaggc |
| 72361 | tggagtgcag tggtgtaatc tcagctcact gcaacctcca cctgccaggt tcaattgatt |
| 72421 | ctcctgcctc agcctcccca gtagctggga ttacaggcac ctgccacatg cctggctaat |
| 72481 | tattgtattt ttagtagaga tggggtttta ccatgttggc caggctggtc tcgatcccct |
| 72541 | gacctcaggt gatccaccca cctcggcctc ccaaagtgct gagatcacag gcgtgagcca |
| 72601 | ccatgcctgg ccgtcacctg gtggtgttga atatgaactg ctgcggtgtt ggtaaattaa |
| 72661 | gcaagcagat agatgtaaat aacgcttggg caggaatatg gagcacggga tgaggatggg |
| 72721 | cggccaactg ttagagaggg tagcagggag gctgagatct gcctgccatg aactgggagg |
| 72781 | agaggctcct ctctctcttc acccccactc tgccccccaa cactcctcag aacttatcct |
| 72841 | ctcctcttct ttccccaggt gaactttgaa ccaggatggc tgagccccgc caggagttcg |
| 72901 | aagtgatgga agatcacgct gggacgtacg ggctggggga caggaaagat caggggggct |
| 72961 | acaccatgca ccaagaccaa gagggtgaca cggacgctgg cctgaaaggt tagtggacag |
| 73021 | ccatgcacag caggcccaga tcactgcaag ccaaggggtg gcgggaacag tttgcatcca |
| 73081 | gaattgcaaa gaaattttaa atacattatt gtcttagact gtcagtaaag taaagcctca |
| 73141 | ttaatttgag tgggccaaga taactcaagc agtgagataa tggccagaca cggtggctca |
| 73201 | cgcctgtaat cccagcactt tggaaggccc aggcaggagg atcccttgag gccaggaatt |
| 73261 | tgagaccggc ctgggcaaca tagcaagacc ccgtctctaa aataatttaa aaattagcca |
| 73321 | ggtgttgtgg tgcatgtcta tagtcctagc tactcaggat gctgaggcag aaggatcact |
| 73381 | tgagcccagg agttcaaggt tgcagtaagc tgtgattata aaactgcact ccagcctgag |
| 73441 | caacagagca agaccctgtc aaaaaaaaaa gaaaagaaaa aagaaagaaa gaaatttacc |
| 73501 | ttgagttacc cacatgagtg aatgtaggga cagagatttt agggccttaa caatctctca |
| 73561 | aatacagggt actttttgag gcattagcca cacctgttag cttataaatc agtggtattg |
| 73621 | attagcatgt aaaatatgtg actttaaaca ttgcttttta tctcttacct agatcaggcc |
| 73681 | tgagtggcct ctctttagca agagttggtt agccctggga ttcttactgt agccacatta |
| 73741 | ataaacaaca tcgacttcta aacattctat aataccatct tttggccaaa ttgacttcgc |
| 73801 | ctcttcctct ctctttccaa atgaaatgtg tttcatttca ctgtcagacc acatggttgg |
| 73861 | ggaccccaca gagcacacag ccctccctct gccttcccat gctggccctt cacccactgc |
| 73921 | tggagtgcca ggttggtcca agggttggac caagttgtct gaggttgtct caaggttggt |
| 73981 | cgaggctgtc tccgcgctgg gttgtgctac aaggagccct tctttccatg ggtgtggctg |
| 74041 | gcagtgagtg ctcacagcaa cagcccacag tgcagcccga gggcaggatg gactcagtcc |
| 74101 | ctgcctccat acccatttct aaggaggcaa aatggcaaac actctacttt tctcttttaa |
| 74161 | tgctaaaaat aagaaaacac cttgcagccc agggtatggg tagtgcatgg aagccgtgga |
| 74221 | gttgtgaggt gggaagtgac ctctgctgga tatgtctatt caggaagatt gctggagtgg |
| 74281 | gtggggtctc tgggaggtcc cctgagtgtg ggaagctggg accaccagct ttctcgcaca |
| 74341 | gggagtggcc atcccagctt ggagaggttc caggactggt tgggaggcac gtttcagatt |
| 74401 | tctatctgtt gaatcagcga agatattgga ttatgaggaa tttgggaatt aggaaagtgg |
| 74461 | gtgcaggtgg gttgggggta ggtgaaggaa gacatgggcg tattggggga gcaggggctg |
| 74521 | ctcagaggtg ttccagaagc tctgggtgag gaggtgagag ggaccgggga atgcagctcg |
| 74581 | gcccagcctc cctgcctgag gtcagccatc acgtggtgat ggcaagatgg aaatgtgctt |
| 74641 | tctgactgct ccagccagtg ctgccagatt cagctcccca gggagggcac ctgagaggct |
| 74701 | ccaagccagg agatctgttt tctcctttgt ttcgtttttt tttgttttgt tttgttttat |
| 74761 | tatactttaa gttctagggt acatgtgcac aacgtgcagg tttgttacat atgtatacat |
| 74821 | gtgccatgtt ggtgtgctgc acccatcaac ttgtcattta cattaggtat atctcctaat |
| 74881 | gctatccctc ccccctcccc ccaccccctg ttttctcctt tgaatccttc ttagaggccg |
| 74941 | ggcgcggtgg ctcacgcctg taatcccagc actttgggag gctgcggcag gaggattgct |
| 75001 | tgagcccagg agttccagac cagcctgggc aacatagtga gacctcgtct ctacagataa |
| 75061 | taattttaaa aattatccgg gcatagtggc atgcacctat agtcccagct actcaagagg |
| 75121 | cagaggcagg aggatcactt gagcccagga ggcggaggtt gccgtgagcc aagatcccac |
| 75181 | cactgcactc cagcctgggc gacagagacc cccatgtcaa ataataataa taataaataa |
| 75241 | atccttctca gtcccttcct cactgtgtcc ccctccactg aatttttcca cctcctctcc |
| 75301 | cacttccccc actcccgctt tccctctcct tctctcccca ctccatcttt ttctttctct |
| 75361 | gctgtttctc gtccctccct cctctccatc ccacaacact gcctaccctg tccctgcccc |
| 75421 | accctggtgc tcaggatgtg tgaagtgagg ggtggtagcc cccaagacct caaccccgaa |
| 75481 | ggttagcctg ttgaaaccac tttctcccag ctgcccccct ggcagttggt gctgctgggg |
| 75541 | gaaactggga ttgggggcca gattttgcct ctcttcctga caaagagaga tgaagagttc |
| 75601 | tctcaccagg tgcctgggac tggggtgtgg gtgtcccagc ctatcccagc gcatctgttc |
| 75661 | tgcatcatga ttaatagtgc tgctttcagc cgggcgcggt ggctcacacc tgtaatccca |
| 75721 | gcactttggg aggctaaggt gggcagatca caaggtcagg agttcgagac cagcctggcc |
| 75781 | aacatggtga aacctcgtct ctactaaaaa tacaaaaatt aaccaggtgt ggtggtgggt |
| 75841 | gcctgtagtc ccagctactt gggaggctga ggcaggagaa tcacttgaat ctgggaagca |
| 75901 | gaggttgcag tgagccaaga tcgtgccact gcactccagc ctgggtgaca gagtgagact |
| 75961 | ccgtcctaaa aaaaaaggag ttttgctctg tcgcccaggc tggagtgtag tggcgccatc |
| 76021 | tcggctcacc gcaacctgcg cctcccgggt gcaagcgatt ctcctgcctc agcctcccaa |
| 76081 | gtagctagga ttacaggcgc ctaccaccac gcctggccag ttcttgtatt tttagaagag |
| 76141 | acggggtttc accctgttgg ccaggctcgt ctgggactcc tgacctcagg taatccgccc |
| 76201 | acctcagcct cccaaagtgc tgggattgca ggcatgagcc accgtgccca gtcaactcct |
| 76261 | tctcaaaaaa aaaaaaatag tgctgctttc tctttcaagt gtcctgattt gggtgatagt |
| 76321 | aaatgccact ctacttataa gggatctacc tcagaatgct aattgggaca tttttgtagc |
| 76381 | actctactgt tggcagcagg tgatgctcac aacagcccgt gagggtggat gacgtccgct |
| 76441 | tcacagatga caaaggagcc tcatgctcag accgtgggct gccagagcag gtccatggct |
| 76501 | gcagccccac atggaccata tttccccctt gtcactcttt ccaccaagct cccttggaac |
| 76561 | ttcagttatt aagctctctt gggtggaatc caagttagaa tcacaacatg tgcctcatat |
| 76621 | ggattgtgcc agtgaaaaat gacattctat ttagaggcag ggcagcctgg cttagagtca |
| 76681 | gtttaaaata tgtattatgc tgcaacaaat gtaccatgat cctgtaagat gttcacaaca |
| 76741 | agggaactgg atgtggggta tactgtctgt actaacttca caagttttct gtaaatctaa |
| 76801 | aactgttcca aaataacaag ttcgtttaaa attaactcca ggagaccagg tacggtagct |
| 76861 | aatgcctata atcccagcac ttcggaaggc tgaggcaggt ggattgcttg agcccaggag |
| 76921 | tttgagacaa gcctgggcaa catggtgaaa tcctgtctct aaaaaaaatc acaaaaatta |
| 76981 | gccaggtgtg gtggcgcatt cctgtagtcc cagctacttg cggggctgag gtgggagaat |
| 77041 | catctgagcc caggagtttg aggctgcagt gagctgtgat tgtaccactg cactccaacc |
| 77101 | tgggcaacag agcaagaccc tgtctcaaaa aacaaaaatg aaataaagtc caggaaagaa |
| 77161 | gtaggtttta ccactcttat tttctgaaga gaaaactaaa tttaatgtgt aaagtgagga |
| 77221 | caagttcacc aagttagtgt ttgagttgcc taaaatatgt ttgctaaaac tattcaaagc |
| 77281 | tttcacataa aacatgatca gaagttctat gccaaaacat atgtgtgtgt atatatatat |
| 77341 | gcactatata tactgtatat aaaaatgcaa aatctaaatt gccaaccttt tagaaattgc |
| 77401 | tctgaaagga aagcatttca agataatttg cttacccaaa gaatatactt tccaagaaag |
| 77461 | caagtaatac ttaaggtgtt cataatcctc atcaaattaa ttcttgctac tgaaagctta |
| 77521 | caaggagctg ttttgatgtc gggtgtgaca ggcttgactt ggcagaaggt gtcactttac |
| 77581 | taacaacatt ttaaataagt gacagaagac aagaaactac acgttaaatg ccagaacaaa |
| 77641 | gagtgtctaa gtggatgcta agagttgaaa tatggctgga tacctgccca agagagctga |
| 77701 | aaagtagatg aaagttggtt acctataaac tagtgcaccc taatgaatta aaaggtgttg |
| 77761 | atgagttaac ttgttatgcc ttccagataa gacatgcaaa tggggcttct tcctccttca |
| 77821 | ctacttccaa gggatttaac aaggagacca atgcaaatga taaggactgt agggctcaag |
| 77881 | ctggggacag attggggaaa gggggaccat catgcccata tagatgtccc tgtgccctgg |
| 77941 | cagtcaaggc tgctgaaaaa taacaaaacc cagaagtctg cgtgatgctg cctctccatt |
| 78001 | tgtccaaagc cttcttgcgg cagtttgcag gcttttgcaa aagctccagg accaaggagc |
| 78061 | tatgttcatg ctggaagctt gttcaggatt agctgttctt tgtgggatgg gtgcagccag |
| 78121 | ggccaggtgt ccagggacag tgttttaaca aagggcatga ggtgtctgat ctcacagtgg |
| 78181 | aactccactt gccttttttt catcttctca ttctgcttca tgcacagaac cagccccatc |
| 78241 | ctgaaactga ctctaaatta ctcccgcccc aggtggagtg cctttctcgg agttcaacag |
| 78301 | agccttcctg tcgcccaagg gacaactcca ctgaatgccc aagccacacc caaaacctaa |
| 78361 | caagtaaaaa ccaaattctg tgctccccca tcctgggcca ttcctggttt ctctactgct |
| 78421 | gttggtgata ccaccatcag cttgtccatc atgaccctgg ccagttcctc ccacaaccct |
| 78481 | ccacagcacc cagggacctc acctccattc catccgacac agatctcctc accacaaacc |
| 78541 | ttggttttgc aacagcagcc atgagacctt tacaccctcc gcccttcatc ctgtccccca |
| 78601 | ctgaggcccc agagccattc cttaaagcag cgcgccacaa actataaccc acaagccaat |
| 78661 | tctggtaccc agcctgtttt gcacagccag tgaactgaca atgatctttt catacagcca |
| 78721 | gaaaaacaaa acaaaacaaa aaacaacaaa aaaaaacccc accattctga gcatgtgact |
| 78781 | tccatgttca agatgtctca tgttcagaaa ggcccctgga aaaggaggaa ggggagctgg |
| 78841 | gcacaaaggg agaccctctc agctgagctc ctcccatcca gacattttcc tggacttcct |
| 78901 | atccaatgac ttcccttagc ttcttatcag ccacccctgt ctgcccagga ggctggaaga |
| 78961 | tgtggccttt taactgggca cagctctgtc ctctatcata tcagggctct gttcccaagg |
| 79021 | agggtagaga gaatggacac caggtggacc ctcagcagtc tgtgccacag agggagtgtt |
| 79081 | tgcaatttcc agactaaaag tccccatgtg cttgacgggg tatgtgacta caacgtgatg |
| 79141 | cttgactttt cctcatatga ccagagccac tttgtccatc tggtacaatg tcagctatct |
| 79201 | gctaggggcc ctccaggatt cccagtcaat tccatatctg catcaccacc attggcacta |
| 79261 | aataaaataa aatactcaag ttcctgctgg tgagcatgag cagtgctaca ctgggccctt |
| 79321 | caaccaaggt gacatgataa tgactgaaaa taatcactgc cacttattgg ggacgtctca |
| 79381 | tctgccaggc atggtacaaa gtgctttaaa taagcattca acaatttcat gctgacagaa |
| 79441 | gccctgtgag ccagtggagc tactactatg cccattatac aggggagaaa actgaggcag |
| 79501 | agagaggtta ggtaattcgc tcagcctcac acaaccaata ggtggtggag ccaggatttg |
| 79561 | ggccccatct gcctgactct ctagaggctc tatcttccag tcttccagag ttgagtctaa |
| 79621 | gccatgaata ggacaattag acagcagagg aaacccattc agccaccatg tgcatgaaga |
| 79681 | gtaaggaatt tctgtcatac agaggggagt gaattcactg agctgagagc tgaggaacca |
| 79741 | ttgatctgat ggctgagaca ccactgggaa gactggagag gcttttctgg gcatgcagtg |
| 79801 | ccaggcacag gaggagctga gggaagatga ctaagaggta ctggcaaaga attcagaaat |
| 79861 | tctgatggaa gctttacatg ttaccatcac atccatccat ctatccaccc atccatccac |
| 79921 | ccatatcttc ctccctccac ccaatcatgc atacatccag tcatctatac accacccacc |
| 79981 | cacccatcca tccatccatc catcccttca tccatcccat catccatcca attatacata |
| 80041 | catccaatca tatatctgta cataatccat tcttccctcg gttcatccat ccatccattc |
| 80101 | atccatccat ccacccatcc cttccttcat ccttcctatc atccatccaa tcatatatct |
| 80161 | gtacataatc cattcttccc tcggttcatc catccatcca ttcatccacc catccaccca |
| 80221 | tcccttcctt catccttcct atcatccatc caatcataca tatatccaat catacatctg |
| 80281 | cacatcacca gctcatccat ctatccattt atccatccat ccttccttcc atccatcatt |
| 80341 | catccatcat acatacatct aaccatacat ctctacatca ttcattcttc catcgattca |
| 80401 | tccaattatc catcattcct tcctccatcc atcccattat ccatttgacc atacatatat |
| 80461 | catctataca tcatccattc atccatccat ccatccatcc acccatatct tcatccaatc |
| 80521 | aatcatacat acatcgaatc atctacacat cacccatcca tccatccatc cattcatcta |
| 80581 | tccacccatc catccatcca tccatccatt catctatcca cccatccatc catccatcca |
| 80641 | tccatccatc catgtaacca tccagtcata tatccaatta cacatccacc cagttataca |
| 80701 | ttcatacatg catctaatca ttcaattata catacacaca tccatataat tctacatcca |
| 80761 | attatacctc catccaatta cacattcata cacccaccta ataaattact aattcatata |
| 80821 | tccatccata taattataca tcaattatac atccatctaa tcattcagta attcacccac |
| 80881 | catccagtca tctatccaat aatacattca tccaatcatc catccatcca tccacccatt |
| 80941 | catccatcca tccgtccgtc cacccatcat ggtatgagcc atgatttacc acgatggtcc |
| 81001 | cctgtggaca gcccaggtgg ggcagaactg aagggaagcc cagggctgcc cccataaaca |
| 81061 | tttgcctcct ttacatggat gagaactaga tccacatgta taaatcctca tgatttgaag |
| 81121 | gtgcttttac caacattcac tcatgggatt ctcccaggag ctctaggagg aggcaggtag |
| 81181 | agttgaggtc atctcacgca ttttacagat gaggaaacgg aggccctgag aggcaggtcc |
| 81241 | aaggccacct gaccagaaag aagtggaact gggacttgaa cccagccatc ttgccccttg |
| 81301 | gtcccatgct ctctagcctg taactcctgc ttcctggtgg ggcatctcca ggaggaccct |
| 81361 | atcggctggc catgggcctg ccctggagtc ttctgctctg tgtggccatc cttcctccct |
| 81421 | caggagagtg tgtgctccca gagcacaggc tgtatcttct gagcattttg tcccttccca |
| 81481 | gtacctagca ctcagctctg tatacattgg gctctcaaga attctcaacc ttccagagtg |
| 81541 | taaggccttg acctgctcag ccctggatac tgcatgatgc attgataagc ccataaaata |
| 81601 | accagggcag attgactccc agtggccaaa gtgccacagg gaagggacaa ttcagccctt |
| 81661 | ctaggaggag gaggaggtag ttttctcatt tctattaagg caacaaaagc tgccttacta |
| 81721 | aggacattct tggtggaggg cgtgactgtc aaccactgtg atcatttggg cctctcttgc |
| 81781 | ccaggcttcc cattctgaaa ggacagtttt attgtaggta cacatggctg ccatttcaaa |
| 81841 | tgtaactcac agcttgtcca tcagtccttg gaggtctttc tatgaaagga gcttggtggc |
| 81901 | gtccaaacac cacccaatgt ccacttagaa gtaagcaccg tgtctgccct gagctgactc |
| 81961 | cttttccaag gaaggggttg gatcgctgag tgtttttcca ggtgtctact tgttgttaat |
| 82021 | taatagcaat gacaaagcag aaggttcatg cgtagctcgg ctttctggta tttgctgccc |
| 82081 | gttgaccaat ggaagataaa cctttgcctc aggtggcacc actagctggt taagaggcac |
| 82141 | tttgtccttt cacccaggag caaacgcaca tcacctgtgt cctcatctga tggccctggt |
| 82201 | gtggggcaca gtcgtgttgg cagggaggga ggtggggttg gtcccctttg tgggtttgtt |
| 82261 | gcgaggccgt gttccagctg tttccacagg gagcgatttt cagctccaca ggacactgct |
| 82321 | ccccagttcc tcctgagaac aaaagggggc gctggggaga ggccaccgct ctgagggctc |
| 82381 | actgtatgtg ttccagaatc tcccctgcag acccccactg aggacggatc tgaggaaccg |
| 82441 | ggctctgaaa cctctgatgc taagagcact ccaacagcgg aaggtgggcc ccccttcaga |
| 82501 | cgccccctcc atgcctccag cctgtgctta gccgtgcttt gagcctccct cctggctgca |
| 82561 | tctgctgctc cccctggctg agagatgtgc tcactccttc ggtgctttgc aggacagcgt |
| 82621 | ggtgggagct gagccttgcg tcgatgcctt gcttgctggt gctgagtgtg ggcaccttca |
| 82681 | tcccgtgtgt gctctggagg cagccaccct tggacagtcc cgcgcacagc tccacaaagc |
| 82741 | cccgctccat acgattgtcc tcccacaccc ccttcaaaag ccccctcctc tctctttctt |
| 82801 | caggggccag taggtcccag agcagccatt tggctgaggg aaggggcagg tcagtggaca |
| 82861 | tctgatcttg gtttagtatc cttcattttg ggggctctgg gtgtggcctg ggcctctgga |
| 82921 | ctttggccac ggtgtttgtt ccagcccttc tcctaacctg tcctttccag acactcggca |
| 82981 | tctaggttat tagcacctcg catactttct gacatgctcc tcagtcctga ttttgaccat |
| 83041 | cttctcttgc ttcccatctg tgtcagtcaa gactgcattt ggctgtaaga aacagaaacc |
| 83101 | ccaactaact gtggcattta catgaagagg tttacttttc tcacataatc agatgtctag |
| 83161 | acttggccag cacctcaagg gtcattgatg ctctcctgtc tttattttct gtcatcttta |
| 83221 | gtggttggat tgttgcctca tggttacaaa gtggctgctg cacttccagg catcacatct |
| 83281 | gcctttgaag caggaacaag ttgcaaagta aagtggccaa aagggccctg aaactaaatg |
| 83341 | tgtcccctta ggaaagcagg agttttcttg caagtggcaa tcttctgctt atgtctcatt |
| 83401 | ggccagagct gggtcttacg gccacccctt gctgcgagca aggctgggac attgagcatt |
| 83461 | ttgccgtcca acctctttag cagaataaac caagggggaa gaacgttaat agtggctttt |
| 83521 | gagtcactag ttggcagtat ctgcccctct atctttccat cctccccatg gagtttcaag |
| 83581 | gttcctttct cagtacttct tcaggctctg cacgttcatt tggatcttgt gtcttggggt |
| 83641 | gaaaaactgg cccaagtgtc tccccaagca tccacctttg gattaatttg gaaaatggct |
| 83701 | gtcaagtgcc cgcctcttgc ttggtataat gctacagctt tagaggacgc agcaggcatg |
| 83761 | ggccttgccg ctgaggttct tagcctcatg agaatatcca gatcagattc tcttggctcc |
| 83821 | ttcttagagc cagtgatgca agacacttcc tgctcatctt gtcgggacgg ttttacaagt |
| 83881 | tgcctgccat cctgagaaag tctacaaaac gatgccagac ctcatgccag cttcccaagc |
| 83941 | cttgactctc agtgctccct caacaggatt ctggaagaat ctcccaaaca agtcgcaatg |
| 84001 | ccctctggac cctgtgcagg catgagactc aagagcattg gctcccaccc ctggtggagg |
| 84061 | gaacactgct ggggctggga tcttgcctgg ttgctccgcc tgcacccaag acaaccataa |
| 84121 | ttaaaatgtc cttcattgaa cttggaaagc cttcaaagct gacaactcct tatgtgtacc |
| 84181 | cggaaaggcc tgggagtgtg ccagggcatt gctcgggagg gacgctgatt tggaagcatt |
| 84241 | tacctgatga gagactgaca gcagctcctg gtagccgagc tttccctcct gcctctgctg |
| 84301 | tgaaggtgga cccatccaac agtcaaatgc ctgactctgg acaggagcgg acctatttat |
| 84361 | tgccatgcaa gggactctgc acttttgaat tgtgggtcat gggcttggat ttaggggtta |
| 84421 | gagctgggag aagtcttgga agtcacctag agatgacact gccattttgc agatgaggaa |
| 84481 | accgtccaat caaaatggac caaggacttg cccaaagcct cacagcaaaa ccataggccc |
| 84541 | ccgcactaac cccagagtcc ctgtgctgtc ttaaggatca tatagttgta agcaatcatc |
| 84601 | tggttttcag tatttcttct tttaaaatgc ctggggccat gcccagcagt ctgtttcact |
| 84661 | gcagcgttta cacagggctg ccgggctttc ctggtggatg agctgggcgg ttcatgagcc |
| 84721 | agaaccactc agcagcatgt cagtgtgctt cctggggagc tggtagcagg ggctccgggc |
| 84781 | cctacttcag ggctgctttc tggcatatgg ctgatcccct cctcactcct cctccctgca |
| 84841 | ttgctcctgc gcaagaagca aaggtgaggg gctgggtatg gctcgtcctg gcccctctaa |
| 84901 | ggtggatctc ggtggtttct agatgtgaca gcacccttag tggatgaggg agctcccggc |
| 84961 | aagcaggctg ccgcgcagcc ccacacggag atcccagaag gaaccacagg tgagggtaag |
| 85021 | ccccagagac ccccaggcag tcaaggccct gctgggtgcc ccagctgacc tgtgacagaa |
| 85081 | gtgagggagc tttgcgtgtt tatcctcctg tggggcagga acatgggtgg attctggctc |
| 85141 | ctgggaatct tgggttgtga gtagctcgat gccttggtgc tcagttacct ccctggctgc |
| 85201 | ctgccagcct ctcagagcat ttagggcctt ctggacttct agatgctcct catcttgcct |
| 85261 | cagtcagcgc gtcagttcca gagacttctc tgcagggttt tctggggcag gtggtggcag |
| 85321 | acccgtgcct tcttgacacc tgaggtcagt ccaccctcct gctcagactg cccagcacag |
| 85381 | ggtcacctcc caaggggtgg accccaagat cacctgagcg cacagagggt gcagatgact |
| 85441 | ggaccacacc ttttggtgat cttaatgagg tggtcccaga ggagctcaga catgcaatct |
| 85501 | agcatccagt tctgggactc tgtctccttt tcaaacgtat tcatgtagaa caggcatgac |
| 85561 | gagaatgcct tgtcaacatg ggtgatgggg aatcaatcag acagggcgcc gggctcaagg |
| 85621 | ctgcagtcac ccaagagtgg ctcagcccac caggccctag gaaacgcctg cacagcctgg |
| 85681 | agctcctgga gtcatttcct tcatgtcttc ttcactgcac ttacgtaaag atgccagcca |
| 85741 | ttggtttggt gatttggagg gtgcccagtt gcccaacaag aaatgcagaa gaggcctagc |
| 85801 | caggatttca ccagcagtgg agagtagaga agatgtggcc agaaaagagt ttcctttccc |
| 85861 | tcctaaagat ggtactccct gcagctactg gggaagcctg cagcattctc tagggctctg |
| 85921 | tgtgttgaga gcagccccac cctggcccct tctgagtgca tttctgcttt gtgacttgat |
| 85981 | ccgtgaagtc ccctgagatg ggcagagggg atgtcctcga agctggggca gagcctcatc |
| 86041 | cttgaacgtg aaggacgttt gaagactgtg gcatgatcac aggatgagat cacagggaac |
| 86101 | ttgagtttct ctcctcctct cccttcacag ttatttcact gagggaaatc cctcccctgc |
| 86161 | ccagaatgaa aactctagcc aactcttgac ttctccatca ctccaaagta gttgaaagta |
| 86221 | cattagtctc cacagtggca aaacagtgtg caaaagctaa ataattagaa cagccagtcc |
| 86281 | catgtgacag tcaaagcttc taactccatt caaagttgca gccattcccc tcgagggctg |
| 86341 | gcagggaggg gaggggtaag agaaacagga aggttcttac tgagttggtc ctggtgtgag |
| 86401 | ctgcgtcaca ctccctgcag aggtttcaag gagactctct ctctctctgt ctccatgggg |
| 86461 | accttatttg aattcttcta ctcttacccc agcctgccat ctccagctat cctcccctga |
| 86521 | agagcccttc tgctgcgctg gattctggtg gccatgtcat ctcctcggcc ccgtgggagt |
| 86581 | ctgaagatct ggctgcagcc tcacctctga ggtcctgcta gttgccacct cttaaacatg |
| 86641 | atctgaggct cccatgcact ctgacctgtg cccacatggg gcccacggga aacacgctgg |
| 86701 | caagcaaact gtgggtgtgc agacggttct cagggctgca gcacctgtcc tttgctctgc |
| 86761 | ccccaaagca aggccagccc atcttccatc ctctagtgtt ccttggtggg gccctgacca |
| 86821 | cagtccacca ggtccctaac cagaggggac acacaccagg tgtcctcaat gtattgcctt |
| 86881 | gaaacagttg tgctgggact gtgatggggg gtggccatgt agccaccccc accaccccca |
| 86941 | agccactctc tccaaggaaa tcctcctaaa gatcccttta catcctccat gtggtgggga |
| 87001 | ggttctagag ttgggtgcat gtgtcttcag ctactgacaa tgcagaccct agttggcacc |
| 87061 | tcgctctggc ctatcctgtt tgctgttctt ggcgctccag tgaaactccc catgggccat |
| 87121 | ccagttgggg tgcagtgtgg ccaccccctt gcaggttcct gccttgctgg agagcacagg |
| 87181 | gccctcctgg ctcttgtaaa acactcccca tggtacagag aggccagcag tgatgtgagg |
| 87241 | cccaacctcc ctccatggtg ttcccaagca gctccctttc tggggtcaag gggtggcaaa |
| 87301 | gacagtgcag cgtccaattt ctgactcaag ccgggcctgg ctatcgcagc tctgcactgt |
| 87361 | gtgtgacagc aaggcaactc acccagtgcc gtggcagtga ccgtgtccga ggaagcctcc |
| 87421 | tcacaccctc tgtctcaagg actctggcat ttagctggac ttgctgtagc tctgagcctt |
| 87481 | tctgccattg ccatcacctt gtcagaaact caggccgaat ctgcactcag agttgtgccc |
| 87541 | aggcagttga gccaacactt gctcagcgat attgtcacat gacaaggcac tgtcaccact |
| 87601 | gggcatcgtg ggtagcgcag tgtcggctgg atggacccgg agggtgtctg tgtcatgcta |
| 87661 | gtgctagtga tgggagcccc gtgagcccat tgcccgccct cccatgccct cagcagctgc |
| 87721 | ctggggacag ccaatggcct gggtgtttct gaggctacca catggcttcc aggaaactcg |
| 87781 | agaacctttc tctcccttgc ctacactctt cacacaggcc tgtgctggcc agcggtgggg |
| 87841 | atccggcatt cctatcttag gtgcagagag tgactgactc attgcaggcc tgggagataa |
| 87901 | gactgatggc ccagccagca agatgtatgg atttctcaga ggcagtggcc tctgtcattg |
| 87961 | tcctcaggaa atgctggtga ttctggtggc ctgaggtcaa tgcatgtcaa cgtggccaac |
| 88021 | ttgccttata aacttttttt ctggacaatt gcgtgcactg tcctgtaaca gtgtcctgtt |
| 88081 | gtttatgatg cagaaatagg tgtttttaaa gcctattgat tttggtacta ttaatgtggt |
| 88141 | caggaacttt ctcagtcttt cttgtttggg gtgagctgtg gcttcctaaa caggaaccca |
| 88201 | agacaccccc aaaagctgct caccagcact gccagcctcc ctcttaccaa gtagcacccg |
| 88261 | ttcaggacat tctgcgaaag gcatttgccc agaagttggg aggaaggaaa tgtaacattt |
| 88321 | tggggcacct accatatgcc aggcaccagg ctaaacgtgt tcacacaaat tctcttacta |
| 88381 | accctcacca tccttctaca agacaaacta gtatcttcat cttggggttc aagatgagga |
| 88441 | aatggaggct cagagaggtt gaatgaatgc cggtgcctgg atatgaaccc catctgcctg |
| 88501 | actccgcaac ccaggcaaag tctttccttg aacttcccag cagccactgc ttagacacag |
| 88561 | cctccacaac catggctcag cagcaaattg cttctctgac ctcactcagc ctgtgtgtcc |
| 88621 | ttgttgagtg aggcattcag gaccctggtc ccaaagtgga gaaagtctct cctactaggt |
| 88681 | catagctaca cctgcatgtg ggtgctgtgc ctcttgttta gtgaacttct atcaccagca |
| 88741 | tcctcagcaa tgacatttgc agagaagcca gagctgaggc accttggtat tcttgggatg |
| 88801 | tgactttcct gaatgtttaa gggaaaatgc ccgaaggtac agagagcttg gtttctagta |
| 88861 | aacaataact gtcttgcttt tacccccctt catttgctga cacatacacc agctgaagaa |
| 88921 | gcaggcattg gagacacccc cagcctggaa gacgaagctg ctggtcacgt gacccaaggt |
| 88981 | cagtgaactg gaattgcctg ccatgacttg ggggttgggg ggagggacat ggggtgggct |
| 89041 | ctgccctgaa aagatcattt ggacctgagc tctaattcac aagtccagga gattttaggg |
| 89101 | agttggttct tatcaaaggt tggctactca gatatagaaa gagccctagt ggtttttttc |
| 89161 | taataccatt tctgggtaat tcctaaggca tttagtgttc tgaaagatgc tagccttgtc |
| 89221 | cagcctggga gttgagaatg aatgtctaac agaaactcta ggccgggcct ggtggctcac |
| 89281 | gcctctaatc ccagcactat gggagaccca ggtgggcaga tcacctgagg tcaggagttt |
| 89341 | gagaccagcc tggccaacat gtgaaatcct gtctcactac aaataaaaaa attagccggg |
| 89401 | tgtggtggta ggtgcctata atcccagcta ctcaggaggc tgaggcagga caatcgctcg |
| 89461 | aacccaggag gtggacgttg cagtgagccg agatcgcatc attgcactcc agcctgggca |
| 89521 | acaaaagcaa aactccgtct caaaaaaaaa aaagaaactc aaatatgtgt gacaggcgat |
| 89581 | tctcactgca ggctgccctg tggctgatcc aggagcaagg ccttaaccat gtcatcccca |
| 89641 | agcgattgct tgtaaacttt cttctgtgca gccttcaacc cttattatga ttttcttctc |
| 89701 | aggaaccaaa ctgctgtatt caagaaaggc agctttgtgt aatcatttat cataaatatc |
| 89761 | ttaagaaaaa tcctagagat tcctaatttt aggaaatggg agacctatgg tactgatata |
| 89821 | atgtgggctg ggcttgtttt ctgtcatttg ctagataaat gaacttgaga gcctactgta |
| 89881 | aaatgtggaa gcttctagat tgcagaaggg ctggaaagac actgttcttt tctcccgagt |
| 89941 | gatgggatct gtccagtatt tagagctgcc tctgaggcca tctgattcta ggagactctg |
| 90001 | cctcgttgag gatattttga ggcctaacta cacattcctg cccccagaga ggtcacagcc |
| 90061 | tatagcaggc tgatgtttct catgtcacat ggcacagaaa ggcacatttt cgttctcagg |
| 90121 | ctaacaaaga gcttcaaaaa ctattagaag ggacagtggc tataagagaa gaacctcagt |
| 90181 | caatgtgtga aattaactag gaacctggct cctgtttctt ttaggtcatg tttttcagct |
| 90241 | taggtaaaac tagaggcttt gataaagcat gacctctaga aatcattgct tttcataaat |
| 90301 | ggaagtgggt ttgagttttt tctactgatt gttagtgcag gtgatgtcta catgccccca |
| 90361 | gaacatattc catgcaacaa aaaaagccca ggtcaccgtc tttgctggga acttgacttt |
| 90421 | tgtgctcact gaattttaag ctttctgaca gcagcctgga atcatggagg gataaagtac |
| 90481 | ctattagtaa gatggaaaaa ggtgtttcag gttggagctg cagtctgttg agagtaagct |
| 90541 | atgggaaggc ctgtatacga ggggtggact tttcttctgt aagtgtccag agaccaggcc |
| 90601 | tcctgaagag ggcatggggg cttaacttac ctggactact gtgtttacaa tactcattta |
| 90661 | tcttgaactc ctcctaaccc ctgagaattg ctacatttag tatttgctga gtacttccta |
| 90721 | gcatcctagg gaatcaatag aacattctcc caaccaggct gggtgcggtg gctcatgtct |
| 90781 | gtaatcccag cactttggga ggccaaggta ggcagatccc ttgaggccag gagtgcaaga |
| 90841 | ctagcctggc tgacatggtg aaaccccgtc tttactaaaa atacaaaagt tagccaggca |
| 90901 | tggtggtaca cacctgtaat cccagctaca tgggaggagt aggaggcagg agaattgctt |
| 90961 | gaacctggga ggtggaggtt gctgtgagcc gagatcatgc cactgcactc cagcctgggc |
| 91021 | gacagagtga gtgagactct gtttaaaaaa aaaaaaaaaa agaacattct cctaacctgg |
| 91081 | cttcttcctc caggggtgta attaatcatg tcagtttcct cattgataca cacacacaca |
| 91141 | cactacaatc ctgtatccat tacttttcaa ggtacattta ctatttacgt ttggggtcct |
| 91201 | tgtctctttt ttaatagtgt ttcttaaagt cttgtattat atcagagtac agtaacatcc |
| 91261 | cagtcaagag cactctagta agctctagga ggaaagcgac ttccggaagg cagtggagac |
| 91321 | ctgtcctgtt ggggcagcat aggggcagcc cctgcctctg gtcagttctg gcgctcaggc |
| 91381 | tcagggttgc ctctgggctg ttcttcccag agactgacaa agggctccca taaggcacct |
| 91441 | gcagagcctg tgagaagctg aagtcaatgt tttcctgaca ccagttgatc tgtgcaggat |
| 91501 | ccattgattt aaccacctgc tgtgtggcat gcactgtggt cgatgccagg aacaggaatt |
| 91561 | ggaggggccc atgagcatgg ccagtatcac aggctggagg tgctgctgcg ctctgaccgg |
| 91621 | gcctcttggg gatgagccca tgtcaaccac cttgcctccg atggggtcgg gcccacaggt |
| 91681 | tacctttgtg tgtccatgac cacaccttcc tccccgacct catccaaatc tctttctttt |
| 91741 | ccaagcccct gaatccttca gggctgcagg ttctgtttaa agcagagctg gtgagttgca |
| 91801 | taggttgttg cattgggact agatggggtg ttcaaagagt tgggagttaa aaaacataaa |
| 91861 | gggtatttat taggagaacc aaggagtgta attctcctgt tcttaatatg cggccaggtt |
| 91921 | aatgaatgtc acgtgaatga accagaaaaa aatgaagtgt gcccttgatc agctgggttg |
| 91981 | gtgtgcagca agctgtgtga ccaggggaca gcagtggtcc tgagggccgt cactgtctgc |
| 92041 | cgtgcagagc ccttcctccc acgggggcct acctcacctg tgccaagggc ttgtctgtgg |
| 92101 | tcagtgacct ggatagatct gaatggggct tctttttcga ggagtcttat ggcaggtctc |
| 92161 | tcagtaaaga ctccattctt gatgatcaca cattttggat tttccaaatc tgtcagagaa |
| 92221 | tgggcttgag gcggggtttg tgggcactag tttcactggt ttcatttacc aaaaagggga |
| 92281 | gcagaagtca agtatggtgg ctcatccctg taatcccaga ggcaagagaa ttgcttgagc |
| 92341 | ccaggagttc gagaccagcc tgagcaacat aaggagaccc cgtctccaca aaaatgaaaa |
| 92401 | ataacatttt agtcagacgt ggtggcatgc atctgtggtc ccagctgctt gggagggtga |
| 92461 | gatgggaggg ttgtttgagc cctggagtta aagttgcaat gagctgtgat tgcaccactg |
| 92521 | cactctagcc tgggtgacag aacgagaccc tgtctcaaaa aaaaaaaaaa agaaagaaag |
| 92581 | aaaggaaaaa aaaaactcat gcctgtaatc ccagcacttt ggggaccggg gtgggcagat |
| 92641 | cacgaggtca ggagatcaag actatcctgg ccaacatggt gaaaccccgt ttctactaaa |
| 92701 | aatacaaaaa ttagccaggt gtggtggcac gtgcctgtaa tcccagttac tcgggaggct |
| 92761 | gaggcaggag aatcgcttga accagggagt cagaggttgc agtgagctga gatcgtgcca |
| 92821 | ctgtactcca gcctgggcga cagagtgaga ctctgtctca aaccaaaaaa aaggggtggg |
| 92881 | gggcgggggc aggagaacag tgagaggtag ggagaggaaa ggggattctc gctacaccca |
| 92941 | aaccagatac catctagagg ctagaatctt tgggaggctc aaattcccta gaaagcagga |
| 93001 | gaagcttctg tagccctccc gctttcccag tagattaagc ccagggcggc tccagatgtg |
| 93061 | tgacatgctc tgtgcccaac cagagcccat cataggcaga ggaataacac ccacaccaga |
| 93121 | agggccctcg gaggtcacca cgtccaagaa ccctctttac agatgaggaa actgaggccc |
| 93181 | agagagggga gagccaccta gcgagctggt ggcggctaga ccaggagagc tgtcattcca |
| 93241 | agcaagcaaa ggcaacgaga cgagcccaga gctgtgctcc catctctttg ttagggggcc |
| 93301 | tgggatgccc tctcagtgtc attttgtcca ggatgatgct ccctctctta agcgattaat |
| 93361 | gcgcccttgc taaccttttg ctatcgctgc ctcttcaaac cagaggagct gagagttccg |
| 93421 | ggccggcaga ggaaggcgcc tgaaaggccc ctggccaatg agattagcgc ccacgtccag |
| 93481 | cctggaccct gcggagaggc ctctggggtc tctgggccgt gcctcgggga gaaagagcca |
| 93541 | gaagctcccg tcccgctgac cgcgagcctt cctcagcacc gtcccgtttg cccagcgcct |
| 93601 | cctccaacag gaggccctca ggagccctcc ctggagtggg gacaaaaagg cggggactgg |
| 93661 | gccgagaagg gtccggcctt tccgaagccc gccaccactg cgtatctcca cacagagcct |
| 93721 | gaaagtggta aggtggtcca ggaaggcttc ctccgagagc caggcccccc aggtctgagc |
| 93781 | caccagctca tgtccggcat gcctggggct cccctcctgc ctgagggccc cagagaggcc |
| 93841 | acacgccaac cttcggggac aggacctgag gacacagagg gcggccgcca cgcccctgag |
| 93901 | ctgctcaagc accagcttct aggagacctg caccaggagg ggccgccgct gaagggggca |
| 93961 | gggggcaaag agaggccggg gagcaaggag gaggtggatg aagaccgcga cgtcgatgag |
| 94021 | tcctcccccc aagactcccc tccctccaag gcctccccag cccaagatgg gcggcctccc |
| 94081 | cagacagccg ccagagaagc caccagcatc ccaggcttcc cagcggaggg tgccatcccc |
| 94141 | ctccctgtgg atttcctctc caaagtttcc acagagatcc cagcctcaga gcccgacggg |
| 94201 | cccagtgtag ggcgggccaa agggcaggat gcccccctgg agttcacgct tcacgtggaa |
| 94261 | atcacaccca acgtgcagaa ggagcaggcg cactcggagg agcatttggg aagggctgca |
| 94321 | tttccagggg cccctggaga ggggccagag gcccggggcc cctctttggg agaggacaca |
| 94381 | aaagaggctg accttccaga gccctctgaa aagcagcctg ctgctgctcc gcgggggaag |
| 94441 | cccgtcagcc gggtccctca actcaaaggt ctgtgtcttg agcttcttcg ctccttccct |
| 94501 | ggggacctcc caggcctccc aggctgcggg cactgccact gagcttccag gcctcccgac |
| 94561 | tcctgctgct tctgacgttc ctaggacgcc actaaatcga cacctgggtg cagctgctcc |
| 94621 | actccctcgg cctcctcccg tgctcaggct gtggccgcac gcgcccctca cgcttgcccg |
| 94681 | ccactctgca tgtcaccagc acccccgctc cgtgctaccc accttgtttg actctctggc |
| 94741 | cacttgattt gtccacaacg gcccatcagc ccacaggagg tttggtgggt gccttccacc |
| 94801 | gacaggatga cgggtgccct catggtgtct agaactctcc aaccctccca tgtaggcata |
| 94861 | agcagcccca ctttgcagat gaggaaacgg aggctcagag aagtacagta acttgccgaa |
| 94921 | ggccaatgag tagtaagtga cagagccagg tttgggatcc aggtaggttg tctctgaaag |
| 94981 | acacgcctgt cctgcatccc acaacgcctc ccaggaggtg ctggagtgtg gacgcctaac |
| 95041 | acagagatgt gcagggcaca cacagcaggt gacacacaca gcatccagag gtggcccaga |
| 95101 | gctcatgctg tgcctttggc ccagtgccct gcccccaccc actctgccct gtggcaggaa |
| 95161 | gacaaggagc agacacaaga tctccctggt ccacatgcca ccacctccct ctgcagagga |
| 95221 | caaggggatc ctcatgctgg cattggaggg ggttgagcag ggcccaccct gagccctcag |
| 95281 | gagcacgacc acagcagccc tgcagggagg gattggtggg aggagagtcc caagtatcag |
| 95341 | ggagaggaga gttggtgtcc cacaggagac ctcagagcca caaggcgagc ttgttcataa |
| 95401 | atttgggacc cttagcattt cacagttatt tgcagagccc agaaatggat gttactgaag |
| 95461 | ctcacagttg caagcatctg ttaaattttt attagatttt acttttaggg aaaactttga |
| 95521 | aatgctataa agaagcctgt gtttaaaagt taagacagag gctgggggcg atggctcacg |
| 95581 | cctgtaatct cagcactttg ggaggccaag gcaggtggat catttgaggt taggagttcg |
| 95641 | agaccagcct ggccaacatg gtgagaccct gtctctacta aaattacaaa aaattagctg |
| 95701 | ggcgtggtgg cgggcacctg tagtcccagc tactggggag gctgaagcag gataagtgct |
| 95761 | tgaacccagg aggcagaggt tacagtgagc caagatcaca ccactgtacc ctaagcctgg |
| 95821 | gcgacagagt gagactctgt ctcaaaaaat aaaataaaat aaagttaaga gagaaaaaaa |
| 95881 | tatatcctat atcctttgtt aaattccaaa acagtagggg acaaataact gacttgacag |
| 95941 | gttactacaa tatttcctga aatgatgttt tcttgaatac tggcctacta gaggttcata |
| 96001 | ggtgtgtttg gattaaaaaa gagttccatg gcccagtgac tgggggaaaa aaataaaaga |
| 96061 | ctaaagtaag ttaaacaggc ttttctgctg caggacttgt cagagccttt aatgtactaa |
| 96121 | tggccattgt gaccctctga gaaggtcaca gagtgggttt cccaaactta cttgattcta |
| 96181 | cctgctaaca tttcctggag gaagtttggg aaatgccgat ttagcagatt cttttgttgt |
| 96241 | gccgtggatg gtgctggttg atgtgggcaa aacaaagaac acgtgagtca gatccgcctg |
| 96301 | gggctcttac taaagtgcag gttcccaggt gccactttag gcttacagac ccagttgtgg |
| 96361 | ggtaagcctg ggagtctttt agcaggtgat tctgccacat agtatagttg gaaaacctct |
| 96421 | gggcatactc attgctggtc cctctagaaa tccaggtgac aatagccaat gagaagctcc |
| 96481 | aagagaccca gttgtccatg gggtagaggg aatgtgatat tgaaaccaaa gaagaaaatc |
| 96541 | tatgatcagt tttcagcagt gactgtcaag agaaggagaa gggtgagtta gcgctgatgc |
| 96601 | tggctgacag gtcagcgggt tggtttcacc aaggagtgtg atgaaggctg atgttgtctg |
| 96661 | tgggaatgta tgatggtaac tggtttgtag ctaatttggg gaagcagtga gaattcgtgc |
| 96721 | cctttgaaga ccagtaagtg gcaagaaacc caccaggcct ggctcagggc tgggctgggc |
| 96781 | ttggctcgtc tcagagcagc tggggctggt ggccaaagcc accattagcg aggggcaggc |
| 96841 | cctgggggta caaccagcaa ctaggggaca aagacaaccc tgccagcctc tcctattctg |
| 96901 | gaggcgtgtg accagaaatg gagatgggtt ggtcagcata agatggccag gaaggtggaa |
| 96961 | atcaggactg ctggcaatct agccacatgg gcaggggagc cgggtggttc caggcagttt |
| 97021 | ccaaggccaa gagggtgagc aggcacctca cagggaatca gggccaagcc tggctgcagt |
| 97081 | gtggagacaa tgcacccacc cccatccttg gatcttgcag gaggctgggt cctcactgag |
| 97141 | ctaccaacat ccatggccct gaggctttta aaacacccat ccatggagtg gggctggtcc |
| 97201 | cagtggggtg aggctgaccc tggcagaaac agggcaggag cctgtgggct agggagactg |
| 97261 | caccttcctt agatagcctc catgccatca tgtccccgtg acagtttctg ctgcgtcccc |
| 97321 | tctgcatggt cccaccctcg gccagcctgc tgccccctct tgccaggtcg cgctaatcag |
| 97381 | tgaccccagt gtgctgtgtt gatactaaca atgcgaggcc tagcagattc aagggaaaag |
| 97441 | agaaccaact gggtttccac cagacccaac taaacaaaca tggacctatc ccagagaaat |
| 97501 | ccagcttcac cacagctggc tttctgtgaa cagtgaaaat ggagtgtgac aagcattctt |
| 97561 | attttatatt ttatcagctc gcatggtcag taaaagcaaa gacgggactg gaagcgatga |
| 97621 | caaaaaagcc aaggtaagct gacgatgcca cggagctctg cagctggtca agtttacaga |
| 97681 | gaagctgtgc tttatgtctg attcattctc atatataatg tggggagtat ttgtcactaa |
| 97741 | agtacagctg tcatttaaag tgctttgtat tttggggcag gcttttaaaa agtccagcat |
| 97801 | ttattagttt tgatacttac cccagggaag agcagttggc aggttcatga agtcatgctc |
| 97861 | ctaattccag ctttcttagt gtactttcag tgagaccctg acagtaaatg aaggtgtgtt |
| 97921 | tgaaaaccaa acccaggaca gtaaatgaag gtgtgtttga aaaccagccc taggacagta |
| 97981 | aatgaagcca tcttctcact gcataaactg cacccagatc ttcgcccatc cttctcagta |
| 98041 | tttcacttca cccattgttt actgtctcaa tgactgggga aatgtctggg gaaatgctcc |
| 98101 | cgtaattgca cagtggcgtt tttcctggaa aatcccacca tggctctaga taagacctat |
| 98161 | ttttcttaaa ggtatctaaa atttccagca taaattctgt ctgaaacacc tgaattttaa |
| 98221 | tcagtactgg agcccggagg gcatctccag ttgccacata gctctgagca ttcagtggtg |
| 98281 | tgttgagggc tgctcccgga agtgcctgca gagtcagggc tccccagcct catctagtga |
| 98341 | ggcagtggaa gggcctgtgg ggatttggag agctggcctg ggtctctgaa gtgatagtga |
| 98401 | cagctgcttg tcaatcacgg tgcacattta gtgctggggg cagggggcag ggaataccag |
| 98461 | cctcatgcat gcatgcattc atttgttcct tccttcattc attcattcag tacacatggg |
| 98521 | tacaacatcc ctgccctgga gttgcccaga gtctagggag gggaaagatc tattaccctg |
| 98581 | ggcctcggcc agctggggag tgctgctggt ggagaggggc cgtgtgcagc gagggaagga |
| 98641 | ggagtcgtca atacccccac cccagctttg ctttcttgtc atcagcccca gggccccagc |
| 98701 | ctgtgtccct cctctcccat tgctacttca tctcctgggt cctccttacc aagcctgacc |
| 98761 | acacagaggg ccttggccgc ttccatgggg aattggaaag caataagata gcatccccta |
| 98821 | gaagcccagt gaagtctggg acaggaccct tctctgagct ctgacttgct cttggaaaca |
| 98881 | cttcgaggct tagcctcccc actttgtttc ccgagagtgt gacctgttcc cctccaaaca |
| 98941 | cccccttctc ctccagggcc atgcccaccc gtcaaaatcc cccacgggca ggacgaactg |
| 99001 | tgggtgtcag tcaccatcta tcctgcatcc tggttccagg gcccccccca gccccgcctc |
| 99061 | catagggaca ggcgtgcaga cacccgtccc tggctgcttc ctcttgtgga atgggttcaa |
| 99121 | aagtaagcag tgttgtttac actgacaaac tgaaaaaaaa agaaaaagag ataacattgg |
| 99181 | aggcttggca cagtggctca tgcctgtaat cccagcactt tgggaggcta aggtgggagg |
| 99241 | atgtccccag cccaagagtt ctagaccagc ctgggcaaca tagcaagacc ccatctcaaa |
| 99301 | aaaaaaattt aattggccag gcagaggtgg gaggatcact tgaacccaaa gggtggaggc |
| 99361 | tgcagtgagc cgtgatggca ccactgcact ccagccaggg caacagaggg agaccctgtc |
| 99421 | tctaaaacaa acaaacaaac aaacaaacaa aagagttaac attggccaga ttaggattca |
| 99481 | ccagatagtg ttaatattag tttgatttga gactttaatc agaaagcaca tgtgtggtgg |
| 99541 | gggtgggtgt aacctaagtc aggtagaatc tttccaactt ggggggggca cactcctgat |
| 99601 | tgtagccata tgagtctgtc agtgtggtgg aagaggccat gggttaatgg gcaggtaaaa |
| 99661 | aagcaccttg cctggaattg agtagaaagt aaggcccttc agaccccgtg acacacttgg |
| 99721 | ggacattttc ttgagtaaca tcctaagatt catgtacctt gatgatctcc atcaacttac |
| 99781 | tcatgtgaag cacctttaaa ccagtcgtct ccaaattcag gggcacagta acatccaaca |
| 99841 | ggctggagaa agaacgtact agaacttcca ttcctttttc atgtcctctt ctaaaagctt |
| 99901 | tgtcagggcc aggcgcggtg gctcacgcct gtaatcccag cactttggga ggccgagacg |
| 99961 | ggtggatcac gaggtcagga gatcgagacc atcctggcta acacagtgaa accccatctc |
| 100021 | tactaaaaat acaaaaaaac gagccgggcg tggtggtggg cgcctgtagt cccagctact |
| 100081 | cgggaggctg aggcaggaga atggcgtgaa cccaggaggc agagcttgca gtgagccgag |
| 100141 | attgcaccac tgcagtccag cctgggcgac agagcgagac tccgtctcaa aaaagaaaaa |
| 100201 | gaaaaagaaa aagaactgtg attggggagg acggtcactt tcctgttctt actgatcaga |
| 100261 | agggatatta agggtacctg attcaaacag cctggagatc actgctttca accattacct |
| 100321 | gccttattta tttttagtta ctgtcctttt ttcagtttgt ttccctcctc catgtgctga |
| 100381 | cttttatttt gattttattt atgtttatgt ttaagacatc cacacgttcc tctgctaaaa |
| 100441 | ccttgaaaaa taggccttgc cttagcccca aacaccccac tcctggtagc tcagaccctc |
| 100501 | tgatccaacc ctccagccct gctgtgtgcc cagagccacc ttcctctcct aaatacgtct |
| 100561 | cttctgtcac ttcccgaact ggcagttctg gagcaaagga gatgaaactc aaggtaagga |
| 100621 | aaccaccttt gaaaagaacc aggctgctct gctgtggttt gcaaatgtgg ggtttgttta |
| 100681 | tttgtttttt agcctcaaag acctttcttc aaatgagttc tggcatagaa gcaccgtgta |
| 100741 | aaatagttag aattctgggc aaaggggaaa agagagctgg gggccatccc tctcagcacc |
| 100801 | ccacaggctc tcatagcagc agctcctaag acacctggtg ggaccttggt ttcgaaatcg |
| 100861 | ctactctaag gctgggcacg gtggctcaca cctgtaatcc cagctcttta ggaggccgag |
| 100921 | gagggtggat cacctgagat caggagttcg agaccagcct ggctaacatg gcaaaaccct |
| 100981 | gtctctacta aaaatacaaa aattagccgg gcgtggtggt atgcgtggtg gtaatcgcag |
| 101041 | ctactcggga ggctgaggca caaggattgc ttgaacccca gaggcagagg ttgtagttag |
| 101101 | ctccagcttg ggcgacagag caagaccctg tcgcaaaaat tgtttaaaaa acaaacccaa |
| 101161 | aattgctact ctcattgggt tcctttgccc attcctgatt ttggcaagag aaatgcttcc |
| 101221 | agattgccct gatctgggta ggacagcatc acgccatagc aacactgccc cgtgagctca |
| 101281 | ctgccccctc aactagcttg tggtccttgg ttaatgtcag tttctttttt gagtttgtgt |
| 101341 | tatgtctaag ggtcatctgc tgggtaacgg aacccaggga ctgccctagt ccctagactg |
| 101401 | tgccatgccc gactctgcca gctttgtcag tgatgctggt gctcgcctcc tcgggtgctc |
| 101461 | gcctggtctg agcacaccca aggagttctt gaggccttag ggttgtttgc gagagaatga |
| 101521 | aagaacacga cctagctctc tttagcatcc ttggtcaggt tcaacactgc ccccaggggc |
| 101581 | ctctggtgga gccaaccacc atcagccaaa taaatccata attagagtca gaaaatggat |
| 101641 | gtctgcatat gtgtagtgca ctaatgtcct gccgatgatt gacatggagt ggagagtgac |
| 101701 | ctgatcattg ctgtgagctc tgctggcctt ggcacaactc atgctgataa ctaatgcaca |
| 101761 | cagttcctct gggaggaaat gtcctcaggg aacttggagt ttgggtgggg atgtgggttt |
| 101821 | gtgtgcccag caagcccttg tggttgtagc agacactagt ggcatctagg aggcaaaggg |
| 101881 | tcaccccagt cttagccacg ttttgagtca aggtggcgga gtggggctgg tgttgactct |
| 101941 | tggtggcagt aacttttccc aatggtgaaa aacccctcta tcatgtttca tttacagggg |
| 102001 | gctgatggta aaacgaagat cgccacaccg cggggagcag cccctccagg ccagaagggc |
| 102061 | caggccaacg ccaccaggat tccagcaaaa accccgcccg ctccaaagac accacccagc |
| 102121 | tctggtaaga agaacgttct cttgaatctt agaggaagct gaagctctca gaggtacagc |
| 102181 | cttcatttta ggaggcctta ggccactgag aatgaataac ccctggcagc tggtcagcag |
| 102241 | cttgcagttt actaagcact ggagtcttca ttgccttctc agtccttttg atttctgagg |
| 102301 | caaatgttga atccctacct tttttttttt ttttcttttg agacagagct tcgcttttgt |
| 102361 | tatccaggcc ggagtgcagt ggtgtgatct cagctcactg catcctccac ctcccaggtt |
| 102421 | caagcgattc tcctacctca gcctccctag tagctgggat tacaggcacc tgccactatg |
| 102481 | cccggctaat tttttgtatt tttagtagag acagggtttc accatgttgg ccaggctggt |
| 102541 | ctcgaacgcc tgacctcagg tgatccacct gcctcggcct cccaaagtgc tgggattaca |
| 102601 | ggcatgagcc accactccca gcctgaatcc tcacttttta tcaatgaaga aattgaggct |
| 102661 | gattctgcag catgataaaa aaaaatacag aaaaaggaaa aaaaagaaag aaatcgagcc |
| 102721 | tctgagagtt tgcttgactg agtctaacca gctcatttta aacccgagga aaatgcagtc |
| 102781 | acatgactac taagtggcag ctctcggagc ctctctggcc ccaagtccag ggttccatag |
| 102841 | aggcagcccc agcatggcat gttttcagtc cccaaatgag actctggaga caaatgtctc |
| 102901 | tggagacaga gcagcagcct ggataagtca caatgggtga cgtcactcag ggctcaaccc |
| 102961 | ctgggcagct taacttgcta gggacgttag gagtctgctg caaaacctga gggtcttagc |
| 103021 | tgagcagtca caggctgggc ccgttgccct gggctcctgt gagtaaaacc cagtcaattt |
| 103081 | tgagtaccca gtaaggcatc cattgagtta ttctgcagcc aggagtgcta ttaagaacag |
| 103141 | tcgcggctgg gcgtggtggc tcatgcctgt aatcccagca ctttgggagg ccaaggtggg |
| 103201 | cggatcacct gaggtcagga gttcgagacc agcttggcca acatggcaaa accccgtctc |
| 103261 | taataaaaat acaaaataat tagctgggcg tggtggcggg cgcctgtaat cccagcttct |
| 103321 | caggagggtg aggaaggaga atcacttgaa cccaggaggc agaggttgca gtgagctgag |
| 103381 | atcgcaccat tgcactccag cctggatgac aaaagtgaga ttccttctca aaaaaaaaaa |
| 103441 | aaaaaaaaca gtcgtcctct ttggggatta gggacagcct gcctgcctgc ccgagcactt |
| 103501 | ctctcttcca ttgccccagt gaagtattcc aggcccctgg gtttagactc tgcaccatgt |
| 103561 | aggggtgtct gacctgcact tgctccttgg tggcacgggc agcctatggc acttgctgcg |
| 103621 | ggctgtgacc aaagcctggc ctggatcttg gatcttggtg actctgcttc tccctggcct |
| 103681 | gagggagctg cccagagcct gcccaccacc tgctgcgtgt ctttgcggtg gcatttctcg |
| 103741 | cacacatgcc gtgcagtggc acccccaagg atggccattc actaaggccc attgtttttg |
| 103801 | tcttttcgct tcgtgttttc tggcctggtg tttttctcat atacatgtga tccagggata |
| 103861 | attaccagaa ttttgacagg attttaagta gcgtttcgat cctgctgttt ttttttcact |
| 103921 | taacatcggg ccagttgact cacactctgt tttttgttgt tgtttttttg agacggagtc |
| 103981 | tcactgtgtc acccaggctg aagtgcagtg gcacaatctt ggcatactgc aacctctgct |
| 104041 | tcccaaattc aagcagtttt cctgcctcag cctcctgagt agctgggact acaggcacag |
| 104101 | gccaccacgc cctgctaatt tttgtatttt tagtaaagac agggtttcac cattttggcc |
| 104161 | agcctagtct cgaactcctg acctcaagtg atccgcccac ctcggcctcc caaagtgctg |
| 104221 | ggattacagg ggactcacac tttgtaacaa cctgaaacaa cgtgatgcat ttccctttgg |
| 104281 | gtcttacctg ctcttcggtg gctgcctgca ggtggagaga ccctccccct tgggcccctc |
| 104341 | gaccttgttt cagaatgggg cccctgctgg gccagctgtg ggtgcctgcc acgtgaagga |
| 104401 | ctcattaagg ccctgtttaa gcctgatgat aataaggctt tcgtggattt ttctctttaa |
| 104461 | gcgactaagc aagtccagag aagaccaccc cctgcagggc ccagatctga gagaggtact |
| 104521 | cgggagccta cttcgctggg agcagcctcc ctttgcgtgt gtggccattc actggcttgt |
| 104581 | gtttctagag ccgggaggac ccttttctgc aatgcagggt tcacacaggg ttcgcagcct |
| 104641 | gaagatggag cagtccgaat tctcttccct gtgcagtttg cgcagctgtg tttgtctgat |
| 104701 | gggctttcta atcctgtgtg ctctccttga cttcagggac aatggcatta caggcatgag |
| 104761 | ccaccatgcc tggctgtctc cctatgtttc agatgaagac ataggcttaa ggaggtcagg |
| 104821 | tgacttgccc acgaccactc tgtaaataag aggcatgaaa agtatttgga gccaccacca |
| 104881 | ccaagcccac tggtcaccct gggtctctga agtcagggag gcaggaggat gggaggtctg |
| 104941 | aggaggcaga gaggctgagc ctggaggccc tggaggccga ggccccatct gttgtttcct |
| 105001 | tatgtggaaa ataagaggct tcgtttgtcc tattgccaca gagcgtacta cttcaggaac |
| 105061 | atccaagaca tggaaatccg cagggcacgg tggctcacgt ctataatccc ggcactttgg |
| 105121 | gaggttgagg tgggagaatc gcttgaggcc agaagttcaa gaccagcccg agcaacatag |
| 105181 | tcagaccccg tctctataaa aaacattatt tttaaaaaag acatggaagt caaattctaa |
| 105241 | aaactggtgc tggctgggtg cggtggctca tgcctataat cccagcactt tgggaggccg |
| 105301 | aggcgggtgg atcacctgag gtcaggagtt caagaccagc ctggccaaca tggtaaaacc |
| 105361 | tctactaaag aaatctttac tgaaaataca aaaatccagt ctctactaaa ataagtctct |
| 105421 | actaaaaata caaaaattag ccaggcgtgg tgctgcacac ctgtaatatc agctactcgg |
| 105481 | gaggctgagg caggagactc gcttgatccc atgcagcgga ggttgcagcg agccgagatc |
| 105541 | acgccattgc actccagcct gggcatcaga ataagactcc gtctcaaaaa aaaaaccaca |
| 105601 | aaaaaacaaa acaacaacaa aagaaaacta gtgcttattc gtcactggcc aagctgccca |
| 105661 | ttggctacat gggtgcttca aagagctgcc cttctccagg tctggccagc aggtatgtgt |
| 105721 | tacagcaaat gcctggggca gcggcagggg cattgctgcg ggaagcttct ggacttgcag |
| 105781 | gaaagctaag ttctcagact gcaggggagc taagcacacc tcggcacagg gtgaggcctg |
| 105841 | cggttctcag acttcagtct ttgtggagct tgagaaaaat gaggctttgc aggtcccacc |
| 105901 | cctagagatt ctgctctatc cactcttgaa ggggatcgag aaatttgcat tttgcaactc |
| 105961 | ccactttcct ccttgaaagc tccggagatt ctgacgcagg gttccgtggg ccacactttg |
| 106021 | gaaaatacag acccatgaga tagaatacca gactgttgaa gtgtaacggg ggcctgggaa |
| 106081 | gtgcagtaac agaagcaagt ttgagggtaa aggacaccca gaggagggag ggacagcatc |
| 106141 | tgcatggaga ggagaagaga ccccccagca gcttccaggg tgttggaagg gtgcgctagt |
| 106201 | aactgctatg catggcaggt ggggaactgt acgtcagggc acagcagcat gaagcggtat |
| 106261 | ggctcgtgtg gacagctagg gacaggcagg cgcggagcag gcatcctgct ctgaaggcca |
| 106321 | aatcccacag aggagccagg gtgctggcag gagccctgaa ctagccgaac agctgaacag |
| 106381 | ctgaacattc accctgtggg gaaagggtca gaagcgtcca ggcttgaggg cacagctggg |
| 106441 | tctcgtcact gcatcaccct tatttaggat aaaggccctg aagaattgta ttagaggttg |
| 106501 | gcaaagcata tctaccacct cctggagcca cgctggccgc agggattata attatttcca |
| 106561 | ttttcaaatt aaggcctctg agctcagaga ggggaagtta cttgtctgag gccacacagc |
| 106621 | ttgttggagc ccatctcttg acccaaagac tgcggagccg agttggccac ctctctggga |
| 106681 | gcgggtattg gatggtggtt gatggttttc cattgctttc ctgggaaagg ggtgtctctg |
| 106741 | tccctaagca aaaaggcagg gaggaagaga tgcttcccca gggcagccgt ctgctgtagc |
| 106801 | tgcgcttcca acctggcttc cacctgccta acccagtggt gagcctggga atggacccac |
| 106861 | gggacaggca gcccccaggg ccttttctga ccccacccac tcgagtcctg gcttcactcc |
| 106921 | cttccttcct tcccaggtga acctccaaaa tcaggggatc gcagcggcta cagcagcccc |
| 106981 | ggctccccag gcactcccgg cagccgctcc cgcaccccgt cccttccaac cccacccacc |
| 107041 | cgggagccca agaaggtggc agtggtccgt actccaccca agtcgccgtc ttccgccaag |
| 107101 | agccgcctgc agacagcccc cgtgcccatg ccagacctga agaatgtcaa gtccaagatc |
| 107161 | ggctccactg agaacctgaa gcaccagccg ggaggcggga aggtgagagt ggctggctgc |
| 107221 | gcgtggaggt gtggggggct gcgcctggag gggtagggct gtgcctggaa gggtagggct |
| 107281 | gcgcctggag gtgcgcggtt gagcgtggag tcgtgggact gtgcatggag gtgtggggct |
| 107341 | ccccgcacct gagcaccccc gcataacacc ccagtcccct ctggaccccc ttcaaggaag |
| 107401 | ttcagttctt tattgggctc tccactacac tgtgagtgcc ctcctcaggc gagagaacgt |
| 107461 | tctggctctt ctcttgcccc ttcagcccct gttaatcgga cagagatggc agggctgtgt |
| 107521 | ctccacggcc ggaggctctc atagtcaggg cacccacagc ggttccccac ctgccttctg |
| 107581 | ggcagaatac actgccaccc ataggtcagc atctccactc gtgggccatc tgcttaggtt |
| 107641 | gggttcctct ggattctggg gagattgggg gttctgtttt gatcagctga ttcttctggg |
| 107701 | agcaagtggg tgctcgcgag ctctccagct tcctaaaggt ggagaagcac agacttcggg |
| 107761 | ggcctggcct ggatcccttt ccccattcct gtccctgtgc ccctcgtctg ggtgcgttag |
| 107821 | ggctgacata caaagcacca cagtgaaaga acagcagtat gcctcctcac tagccaggtg |
| 107881 | tgggcgggtg ggtttcttcc aaggcctctc tgtggccgtg ggtagccacc tctgtcctgc |
| 107941 | accgctgcag tcttccctct gtgtgtgctc ctggtagctc tgcgcatgct catcttctta |
| 108001 | taagaacacc atggcagctg ggcgtagtgg ctcacgccta taatcccagc actttgggag |
| 108061 | gctgaggcag gcagatcacg aggtcaggag ttcgagacca acctgaccaa cagggtgaaa |
| 108121 | cctcgtctct actaaaaata caaaaatacc tgggcgtggt ggtggtgcgc gcctataatc |
| 108181 | ccagctactc aggaggctga ggcaggagaa tcgcttgaac ccaggaggca gaggttgcag |
| 108241 | tgagccgaga tagtgccact gcactccagt ttgagcaaca gagcgagact ctgtctcaaa |
| 108301 | acaaaataaa acaaaccaaa aaaacccacc atggcttagg gcccagcctg atgacctcat |
| 108361 | ttttcactta gtcacctctc taaaggccct gtctccaaat agagtcacat tctaaggtac |
| 108421 | gggggtgttg gggagggggg ttagggcttc aacatgtgaa tttgcgggga ccacaattca |
| 108481 | gcccaggacc ccgctcccgc cacccagcac tggggagctg gggaagggtg aagaggaggc |
| 108541 | tgggggtgag aaggaccaca gctcactctg aggctgcaga tgtgctgggc cttctgggca |
| 108601 | ctgggcctcg gggagctagg gggctttctg gaaccctggg cctgcgtgtc agcttgcctc |
| 108661 | ccccacgcag gcgctctcca caccattgaa gttcttatca cttgggtctg agcctggggc |
| 108721 | atttggacgg agggtggcca ccagtgcaca tgggcacctt gcctcaaacc ctgccacctc |
| 108781 | cccccaccca ggatcccccc tgcccccgaa caagcttgtg agtgcagtgt cacatcccat |
| 108841 | cgggatggaa atggacggtc gggttaaaag ggacgcatgt gtagaccctg cctctgtgca |
| 108901 | tcaggcctct tttgagagtc cctgcgtgcc aggcggtgca cagaggtgga gaagactcgg |
| 108961 | ctgtgcccca gagcacctcc tctcatcgag gaaaggacag acagtggctc ccctgtggct |
| 109021 | gtggggacaa gggcagagct ccctggaaca caggagggag ggaaggaaga gaacatctca |
| 109081 | gaatctccct cctgatggca aacgatccgg gttaaattaa ggtccggcct tttcctgctc |
| 109141 | aggcatgtgg agcttgtagt ggaagaggct ctctggaccc tcatccacca cagtggcctg |
| 109201 | gttagagacc ttggggaaat aactcacagg tgacccaggg cctctgtcct gtaccgcagc |
| 109261 | tgagggaaac tgtcctgcgc ttccactggg gacaatgcgc tccctcgtct ccagactttc |
| 109321 | cagtcctcat tcggttctcg aaagtcgcct ccagaagccc catcttggga ccaccgtgac |
| 109381 | tttcattctc cagggtgcct ggccttggtg ctgcccaaga ccccagaggg gccctcactg |
| 109441 | gcctttcctg ccttttctcc cattgcccac ccatgcaccc ccatcctgct ccagcaccca |
| 109501 | gactgccatc caggatctcc tcaagtcaca taacaagcag cacccacaag gtgctccctt |
| 109561 | ccccctagcc tgaatctgct gctccccgtc tggggttccc cgcccatgca cctctggggg |
| 109621 | cccctgggtt ctgccatacc ctgccctgtg tcccatggtg gggaatgtcc ttctctcctt |
| 109681 | atctcttccc ttcccttaaa tccaagttca gttgccatct cctccaggaa gtcttcctgg |
| 109741 | attcccctct ctcttcttaa agcccctgta aactctgacc acactgagca tgtgtctgct |
| 109801 | gctccctagt ctgggccatg agtgagggtg gaggccaagt ctcatgcatt tttgcagccc |
| 109861 | ccacaagact gtgcaggtgg ccggccctca ttgaatgcgg ggttaattta actcagcctc |
| 109921 | tgtgtgagtg gatgattcag gttgccagag acagaaccct cagcttagca tgggaagtag |
| 109981 | cttccctgtt gaccctgagt tcatctgagg ttggcttgga aggtgtgggc accatttggc |
| 110041 | ccagttctta cagctctgaa gagagcagca ggaatggggc tgagcaggga agacaacttt |
| 110101 | ccattgaagg cccctttcag ggccagaact gtccctccca ccctgcagct gccctgcctc |
| 110161 | tgcccatgag gggtgagagt caggcgacct catgccaagt gtagaaaggg gcagatggga |
| 110221 | gccccaggtt atgacgtcac catgctgggt ggaggcagca cgtccaaatc tactaaaggg |
| 110281 | ttaaaggaga aagggtgact tgacttttct tgagatattt tgggggacga agtgtggaaa |
| 110341 | agtggcagag gacacagtca cagcctccct taaatgccag gaaagcctag aaaaattgtc |
| 110401 | tgaaactaaa cctcagccat aacaaagacc aacacatgaa tctccaggaa aaaagaaaaa |
| 110461 | gaaaaatgtc atacagggtc catgcacaag agcctttaaa atgacccgct gaagggtgtc |
| 110521 | aggcctcctc ctcctggact ggcctgaagg ctccacgagc ttttgctgag acctttgggt |
| 110581 | ccctgtggcc tcatgtagta cccagtatgc agtaagtgct caataaatgt ttggctacaa |
| 110641 | aagaggcaaa gctggcggag tctgaagaat ccctcaaccg tgccggaaca gatgctaaca |
| 110701 | ccaaagggaa aagagcagga gccaagtcac gtttgggaac ctgcagaggc tgaaaactgc |
| 110761 | cgcagattgc tgcaaatcat tgggggaaaa acggaaaacg tctgttttcc cctttgtgct |
| 110821 | tttctctgtt ttcttctttg tgcttttctc tgttttcagg atttgctaca gtgaacatag |
| 110881 | attgctttgg ggccccaaat ggaattattt tgaaaggaaa atgcagataa tcaggtggcc |
| 110941 | gcactggagc accagctggg taggggtaga gattgcaggc aaggaggagg agctgggtgg |
| 111001 | ggtgccaggc aggaagagcc cgtaggcccc gccgatcttg tgggagtcgt gggtggcagt |
| 111061 | gttccctcca gactgtaaaa gggagcacct ggcgggaaga gggaattctt ttaaacatca |
| 111121 | ttccagtgcc cgagcctcct ggacctgttg tcatcttgag gtgggcctcc cctgggtgac |
| 111181 | tctagtgtgc agcctggctg agactcagtg gccctgggtt cttactgctg acacctaccc |
| 111241 | tcaacctcaa ccactgcggc ctcctgtgca ccctgatcca gtggctcatt ttccactttc |
| 111301 | agtcccagct ctatccctat ttgcagtttc caagtgcctg gtcctcagtc agctcagacc |
| 111361 | cagccaggcc agcccctggt tcccacatcc cccttgccaa gctcatcccc gccctgtttg |
| 111421 | gcctgcggga gtgggagtgt gtccagacac agagacaaag gaccagcttt taaaacattt |
| 111481 | tgttggggcc aggtgtggtg gctcacacct aatcccaaca cctggggagg ccaaggcaga |
| 111541 | aggatcactt gagtccagga gttcaagacc agcctgggca acatagggag accctgtctc |
| 111601 | tacaattttt tttttaatta gctgggcctg ttggcactct cctgtagttc cagctactct |
| 111661 | agaggctgag gtgggaggac tgcttgagcc tgggaggtca gggctgcaat gagccatgtt |
| 111721 | cacaccactg aacgccagcc tgggcgagac cctgtatcaa aaaagtaaag taaaatgaat |
| 111781 | cctgtacgtt atattaaggt gccccaaatt gtacttagaa ggatttcata gttttaaata |
| 111841 | cttttgttat ttaaaaaatt aaatgactgc agcatataaa ttaggttcct aatggagggg |
| 111901 | aaaaagagta caagaaaaga aataagaatc tagaaacaaa gataagagca gaaataaacc |
| 111961 | agaaaacaca accttgcact cctaacttaa aaaaaaaaat gaagaaaaca caaccagtaa |
| 112021 | aacaacatat aacagcatta agagctggct cctggctggg cgcggtggcg catgcctgta |
| 112081 | atcccaacac tttgggaggc cgatgctgga ggatcacttg agaccaggag ttcaaggttg |
| 112141 | cagtgagcta tgatcatacc actacaccct agcctgggca acacagtgag actgagactc |
| 112201 | tattaaaaaa aaaatgctgg ttccttcctt atttcattcc tttattcatt cattcagaca |
| 112261 | acatttatgg ggcacttctg agcaccaggc tctgtgctaa gagcttttgc ccccagggtc |
| 112321 | caggccaggg gacaggggca ggtgagcaga gaaacagggc cagtcacagc agcaggagga |
| 112381 | atgtaggatg gagagcttgg ccaggcaagg acatgcaggg ggagcagcct gcacaagtca |
| 112441 | gcaagccaga gaagacaggc agacccttgt ttgggacctg ttcagtggcc tttgaaagga |
| 112501 | cagcccccac ccggagtgct gggtgcagga gctgaaggag gatagtggaa cactgcaacg |
| 112561 | tggagctctt cagagcaaaa gcaaaataaa caactggagg cagctggggc agcagagggt |
| 112621 | gtgtgttcag cactaagggg tgtgaagctt gagcgctagg agagttcaca ctggcagaag |
| 112681 | agaggttggg gcagctgcaa gcctctggac atcgcccgac aggacagagg gtggtggacg |
| 112741 | gtggccctga agagaggctc agttcagctg gcagtggccg tgggagtgct gaagcaggca |
| 112801 | ggctgtcggc atctgctggg gacggttaag caggggtgag ggcccagcct cagcagccct |
| 112861 | tcttgggggg tcgctgggaa acatagagga gaactgaaga agcagggagt cccagggtcc |
| 112921 | atgcagggcg agagagaagt tgctcatgtg gggcccaggc tgcaggatca ggagaactgg |
| 112981 | ggaccctgtg actgccagcg gggagaaggg ggtgtgcagg atcatgccca gggaagggcc |
| 113041 | caggggccca agcatggggg ggcctggttg gctctgagaa gatggagcta aagtcacttt |
| 113101 | ctcggaggat gtccaggcca atagttggga tgtgaagacg tgaagcagca cagagcctgg |
| 113161 | aagcccagga tggacagaaa cctacctgag cagtggggct ttgaaagcct tggggcgggg |
| 113221 | ggtgcaatat tcaagatggc cacaagatgg caatagaatg ctgtaacttt cttggttctg |
| 113281 | ggccgcagcc tgggtggctg cttccttccc tgtgtgtatt gatttgtttc tcttttttga |
| 113341 | gacagagtct tgctgggttg cccaggctgg agtgcagtgg tgcgatcata gctcactgca |
| 113401 | gccttgaagt cctgagctca agagatcctt ccacctcagc ctcctgagta gttgggacca |
| 113461 | caggcttgca ccacagtgcc caactaattt cttatatttt ttgtagagat ggggtttcac |
| 113521 | tgtgtcgccc aggatggtct tgaactcctg ggctcaagtg atcctcctgc ctcagcctcg |
| 113581 | caaattgctg ggattacagg tgtgagccac catgcccgac cttctcttct taagggcgtg |
| 113641 | tgtgtgtgtg tgtgtgtgtg ggcgcactct cgtcttcacc ttcccccagc cttgctctgt |
| 113701 | ctctacccag tcacctctgc ccatctctcc gatctgtttc tctctccttt tacccctctt |
| 113761 | tcctccctcc tcatacacca ctgaccatta tagagaactg agtattctaa aaatacattt |
| 113821 | tatttattta ttttgagaca gagtctcact ctgtcaccca ggctggagtg cagtggtgca |
| 113881 | atctcggctc actgcaacct ccgcctccca ggctgaagca actctcctgc ctcagcctcc |
| 113941 | ctagtagctg ggattacaag cacacaccac catgcctagc aaatttttat atttttagta |
| 114001 | gaggaggggt gtcaccatgt ttgccaagct ggtctcaaac tcctggcccc aggtgatctg |
| 114061 | cctaccttgg tctcccaaag tgctgggatt acaggtgtga gccaccacgc ctgcccttaa |
| 114121 | aaatacatta tatttaatag caaagcccca gttgtcactt taaaaagcat ctatgtagaa |
| 114181 | catttatgtg gaataaatac agtgaatttg tacgtggaat cgtttgcctc tcctcaatca |
| 114241 | gggccaggga tgcaggtgag cttgggctga gatgtcagac cccacagtaa gtggggggca |
| 114301 | gagccaggct gggaccctcc tctaggacag ctctgtaact ctgagaccct ccaggcatct |
| 114361 | tttcctgtac ctcagtgctt ctgaaaaatc tgtgtgaatc aaatcattct aaaggagctt |
| 114421 | gggttcatca ctgtttaaag gacagtgtaa ataattctga aggtgactct accctgttat |
| 114481 | ttgatctctt ctttggccag ctgacttaac aggacataga caggttttcc tgtgtcagtt |
| 114541 | cctaagctga tcaccttgga cttgaagagg aggcttgtgt gggcatccag tgcccacccc |
| 114601 | gggttaaact cccagcagag tattgcactg ggcttgctga gcctggtgag gcaaagcaca |
| 114661 | gcacagcgag caccaggcag tgctggagac aggccaagtc tgggccagcc tgggagccaa |
| 114721 | ctgtgaggca cggacggggc tgtggggctg tggggctgca ggcttggggc cagggaggga |
| 114781 | gggctgggct ctttggaaca gccttgagag aactgaaccc aaacaaaacc agatcaaggt |
| 114841 | ctagtgagag cttagggctg ctttgggtgc tccaggaaat tgattaaacc aagtggacac |
| 114901 | acacccccag ccccacctca ccacagcctc tccttcaggg tcaaactctg accacagaca |
| 114961 | tttctcccct gactaggagt tccctggatc aaaattggga gcttgcaaca catcgttctc |
| 115021 | tcccttgatg gtttttgtca gtgtctatcc agagctgaag tgtaatatat atgttactgt |
| 115081 | agctgagaaa ttaaatttca ggattctgat ttcataatga caaccattcc tcttttctct |
| 115141 | cccttctgta aatctaagat tctataaacg gtgttgactt aatgtgacaa ttggcagtag |
| 115201 | ttcaggtctg ctttgtaaat acccttgtgt ctattgtaaa atctcacaaa ggcttgttgc |
| 115261 | cttttttgtg gggttagaac aagaaaaagc cacatggaaa aaaaatttct tttttgtttt |
| 115321 | tttgtttgct tgtttttttg agacagagtt tcactctgtc gcccaggctg gagtgcagtg |
| 115381 | gtgcgatctc cgcccactgc aagctccacc tcccgggttc atgctattct cctgtctcag |
| 115441 | cctcccaagt agctgggact gcaggtgccc gccaccacac ctggctaatt tttttgtatt |
| 115501 | tttagtagag acggggtttc accgtgttag ccaggatggt ctcaatctcc tgacctcgtc |
| 115561 | atctgcctgc ctcggcctcc caaagtgctg agattacagg cgtgagccac cgtgcccggc |
| 115621 | cagaaaaaaa catttctaag tatgtggcag atactgaatt attgcttaat gtcctttgat |
| 115681 | tcatttgttt aatttcttta atggattagt acagaaaaca aagttctctt ccttgaaaaa |
| 115741 | ctggtaagtt ttctttgtca gataaggaga gttaaataac ccatgacatt tccctttttg |
| 115801 | cctcggcttc caggaagctc aaagttaaat gtaatgatca ctcttgtaat tatcagtgtt |
| 115861 | gatgcccttc ccttcttcta atgttactct ttacattttc ctgctttact attgtgtgtg |
| 115921 | ttttctaatt ctaagctgtt cccactcctt tctgaaagca ggcaaatctt ctaagcctta |
| 115981 | tccactgaaa agttatgaat aaaaaatgat cgtcaagcct acaggtgctg aggctactcc |
| 116041 | agaggctgag gccagaggac cacttgagcc caggaatttg agacctgggc tgggcagcat |
| 116101 | agcaagactc tatctccatt aaaactattt ttttttattt aaaaaataat ccgcaaagaa |
| 116161 | ggagtttatg tgggattcct taaaatcgga gggtggcatg aattgattca aagacttgtg |
| 116221 | cagagggcga cagtgactcc ttgagaagca gtgtgagaaa gcctgtccca cctccttccg |
| 116281 | cagctccagc ctgggctgag gcactgtcac agtgtctcct tgctggcagg agagaatttc |
| 116341 | aacattcacc aaaaagtagt attgttttta ttaggtttat gaggctgtag ccttgaggac |
| 116401 | agcccaggac aactttgttg tcacatagat agcctgtggc tacaaactct gagatctaga |
| 116461 | ttcttctgtg gctgcttctg acctgagaaa gttgcggaac ctcagcgagc ctcacatggc |
| 116521 | ctccttgtcc ttaacgtggg gacggtgggc aagaaaggtg atgtggcact agagatttat |
| 116581 | ccatctctaa aggaggagtg gattgtacat tgaaacacca gagaaggaat tacaaaggaa |
| 116641 | gaatttgagt atctaaaaat gtaggtcagg cgctcctgtg ttgattgcag ggctattcac |
| 116701 | aatagccaag atttggaagc aacccaagtg tccatcaaca gacaaatgga taaagaaaat |
| 116761 | gtggtgcata tacacaatgg aatactattc agccatgaaa aagaatgaga atctgtcatt |
| 116821 | tgaaacaaca tggatggaac tggaggacat tatgttaagt gaaataagcc agacagaagg |
| 116881 | acagacttca catgttctca cacatttgtg ggagctaaaa attaaactca tggagataga |
| 116941 | gagtagaagg atggttacca gaggctgagg agggtggagg ggagcaggga gaaagtaggg |
| 117001 | atggttaatg ggtacaaaaa cgtagttagc atgcatagat ctagtattgg atagcacagc |
| 117061 | agggtgacga cagccaacag taatttatag tacatttaaa aacaactaaa agagtgtaat |
| 117121 | tggactggct aacatggtga aaccccgtct ctactaaaaa tacaaaaatt agctgggcat |
| 117181 | ggtggctcac gcctgtaatc ccagcacttt gggaggccga ggcgggccga tcacgaggtc |
| 117241 | aggagatcga gaccatccta gctaacatgg tgaaaccccg tctctactac aaatacaaaa |
| 117301 | aaaagaaaaa attagccggg catggtggtg ggcgcctgta gtcccagcta ctcgggaggc |
| 117361 | tgaggcagga gaatggcgtg aacccgggag gcggagcttg cagtgagccg agatcgcgcc |
| 117421 | actgcactcc agcctgggcg acaaggcaag attctatctc aaaaaaataa aaataaaata |
| 117481 | aaataaaata ataaaataaa ataaaataaa ataaaataaa taaaataaaa taaaatgtat |
| 117541 | aattggaatg tttataacac aagaaatgat aaatgcttga ggtgatagat accccattca |
| 117601 | ccgtgatgtg attattgcac aatgtatgtc tgtatctaaa tatctcatgt accccacaag |
| 117661 | tatatacacc tactatgtac ccatataaat ttaaaattaa aaaattataa aacaaaaata |
| 117721 | aataagtaaa ttaaaatgta ggctggacac cgtggttcac gcctgtaatc ccagtgcttt |
| 117781 | gtgaggctga ggtgagagaa tcacttgagc ccaggagttt gagaccggcc tgggtgacat |
| 117841 | agcgagaccc catcatcaca aagaattttt aaaaattagc tgggcgtggt agcacatacc |
| 117901 | ggtagttcca gctacttggg agaccgaggc aggaggattg cttgagccca ggagtttaag |
| 117961 | gctgcagtga gctacgatgg cgccactgca ttccagcctg ggtgacagag tgagagcttg |
| 118021 | tctctatttt aaaaataata aaaagaataa ataaaaataa attaaaatgt aaatatgtgc |
| 118081 | atgttagaaa aaatacaccc atcagcaaaa agggggtaaa ggagcgatct cagtcataat |
| 118141 | tggagagatg cagaataagc cagcaatgca gtttctttta ttttggtcaa aaaaaataag |
| 118201 | caaaacaatg ttgtaaacac ccagtgctgg cagcaatgtg gtgaggctgg ctctctcacc |
| 118261 | agggctcaca gggaaaactc atgcaaccct tttagaaagc catgtggaga gttgtaccga |
| 118321 | gaggttttag aatatttata actttgaccc agaaattcta ttctaggact ctgtgttatg |
| 118381 | aaaataaccc atcatatgga aaaagctcct ttcagaaaga ggttcatggg aggctgtttg |
| 118441 | tatttttttt ttctttgcat caaatccagc tcctgcagga ctgtttgtat tattgaagta |
| 118501 | caaagtggaa tcaatacaaa tgttggatag caggggaaca atattcacaa aatggaatgg |
| 118561 | gacatagtat taaacatagt gcttctgatg accgtagacc atagacaatg cttaggatat |
| 118621 | gatatcactt cttttgttgt tttttgtatt ttgagacgaa gtctcattct gtcacccagg |
| 118681 | ctggagttca gtggcgccat ctcagctcac tgcaacctcc atctcccggg ttcaagctat |
| 118741 | tctccttcct caacctcccg agtagctggg ttgcgcacca ccatgcctgg ctaacttttg |
| 118801 | tatttttagt acagacgggg tttcaccacg ttggccaggc tgctcttgaa ctcctgacgt |
| 118861 | caggtgatcc accagccttg acctcccaaa gtgctaggat tacaggagcc actgtaccca |
| 118921 | gcctaggata tgatatcact tcttagagca agatacaaaa ttgcatgtgc acaataattc |
| 118981 | taccaagtat aggtatacag gggtagttat atataaatga gacttcaagg aaatacaaca |
| 119041 | aaatgcaatc gtgattgtgt tagggtggta agaaaacggt ttttgcttcg atgagctctg |
| 119101 | ttttttaaaa tcgttatatt ttctaataaa aatacatagt cttttgaagg aacataaaag |
| 119161 | attatgaaga aatgagttag atattgattc ctattgaaga ttcagacaag taaaattaag |
| 119221 | gggaaaaaaa acgggatgaa ccagaagtca ggctggagtt ccaaccccag atccgacagc |
| 119281 | ccaggctgat ggggcctcca gggcagtggt ttccacccag cattctcaaa agagccactg |
| 119341 | aggtctcagt gccattttca agatttcgga agcggcctgg gcacggctgg tccttcactg |
| 119401 | ggatcaccac ttggcaatta tttacacctg agacgaataa aaaccagagt gctgagatta |
| 119461 | caggcatggt ggcttacgct tgtaatcggc ttcgggaagc cgaggtgggc tgattgcttg |
| 119521 | agcccaggag tttcaaacta tcctggacaa catagcatga cctcgtctct acaaaaaata |
| 119581 | caaaaaattt gccaggtgtg gtggcatgtg cctgtggtcc cagctactcg ggaggctgaa |
| 119641 | gtaggagaat cccctgagcc ctgggaagtc gaggctgcac tgagccgtga tggtgtcact |
| 119701 | gcactccagc ctgggtgaca aagtgagacc ctatctcaca aagaaaaaaa acaaaacaaa |
| 119761 | aaacccaaag cacactgttt ccactgtttc cagagttcct gagaggaaag gtcaccgggt |
| 119821 | gaggaagacg ttctcactga tctggcagag aaaatgtcca gtttttccaa ctccctaaac |
| 119881 | catggttttc tatttcatag ttcttaggca aattggtaaa aatcatttct catcaaaacg |
| 119941 | ctgatatttt cacacctccc tggtgtctgc agaaagaacc ttccagaaat gcagtcgtgg |
| 120001 | gagacccatc caggccaccc ctgcttatgg aagagctgag aaaaagcccc acgggagcat |
| 120061 | ttgctcagct tccgttacgc acctagtggc attgtgggtg ggagagggct ggtgggtgga |
| 120121 | tggaaggaga aggcacagcc cccccttgca gggacagagc cctcgtacag aagggacacc |
| 120181 | ccacatttgt cttccccaca aagcggcctg tgtcctgcct acggggtcag ggcttctcaa |
| 120241 | acctggctgt gtgtcagaat caccagggga acttttcaaa actagagaga ctgaagccag |
| 120301 | actcctagat tctaattcta ggtcagggct aggggctgag attgtaaaaa tccacaggtg |
| 120361 | attctgatgc ccggcaggct tgagaacagc cgcagggagt tctctgggaa tgtgccggtg |
| 120421 | ggtctagcca ggtgtgagtg gagatgccgg ggaacttcct attactcact cgtcagtgtg |
| 120481 | gccgaacaca tttttcactt gacctcaggc tggtgaacgc tcccctctgg ggttcaggcc |
| 120541 | tcacgatgcc atccttttgt gaagtgagga cctgcaatcc cagcttcgta aagcccgctg |
| 120601 | gaaatcactc acacttctgg gatgccttca gagcagccct ctatccctcc agctcccctg |
| 120661 | ggatgtgact caacctcccg tcactcccca gactgcctct gccaagtccg aaagtggagg |
| 120721 | catccttgcg agcaagtagg cgggtccagg gtggcgcatg tcactcatcg aaagtggagg |
| 120781 | cgtccttgcg agcaagcagg cgggtccagg gtggcgtgtc actcatcctt ttttctggct |
| 120841 | accaaaggtg cagataatta ataagaagct ggatcttagc aacgtccagt ccaagtgtgg |
| 120901 | ctcaaaggat aatatcaaac acgtcccggg aggcggcagt gtgagtacct tcacacgtcc |
| 120961 | catgcgccgt gctgtggctt gaattattag gaagtggtgt gagtgcgtac acttgcgaga |
| 121021 | cactgcatag aataaatcct tcttgggctc tcaggatctg gctgcgacct ctgggtgaat |
| 121081 | gtagcccggc tccccacatt cccccacacg gtccactgtt cccagaagcc ccttcctcat |
| 121141 | attctaggag ggggtgtccc agcatttctg ggtcccccag cctgcgcagg ctgtgtggac |
| 121201 | agaatagggc agatgacgga ccctctctcc ggaccctgcc tgggaagctg agaataccca |
| 121261 | tcaaagtctc cttccactca tgcccagccc tgtccccagg agccccatag cccattggaa |
| 121321 | gttgggctga aggtggtggc acctgagact gggctgccgc ctcctccccc gacacctggg |
| 121381 | caggttgacg ttgagtggct ccactgtgga caggtgaccc gtttgttctg atgagcggac |
| 121441 | accaaggtct tactgtcctg ctcagctgct gctcctacac gttcaaggca ggagccgatt |
| 121501 | cctaagcctc cagcttatgc ttagcctgcg ccaccctctg gcagagactc cagatgcaaa |
| 121561 | gagccaaacc aaagtgcgac aggtccctct gcccagcgtt gaggtgtggc agagaaatgc |
| 121621 | tgcttttggc ccttttagat ttggctgcct cttgccagga gtggtggctc gtgcctgtaa |
| 121681 | ttccagcact ttgggagact aaggcgggag gttcgcttga gcccaggagt tcaagaccag |
| 121741 | cctgggcaac aatgagaccc ctgtgtctac aaaaagaatt aaaattagcc aggtgtggtg |
| 121801 | gcacgcacct gtagtcccag ctacttggga ggctgaggtg ggaggattgc ctgagtccgg |
| 121861 | gaggcggaag ttgcaaggag ccatgatcgc gccactgcac ttcaacctag gcaacagagt |
| 121921 | gagactttgt ctcaaaaaac aatcatataa taattttaaa ataaatagat ttggcttcct |
| 121981 | ctaaatgtcc ccggggactc cgtgcatctt ctgtggagtg tctccgtgag attcgggact |
| 122041 | cagatcctca agtgcaactg acccacccga taagctgagg cttcatcatc ccctggccgg |
| 122101 | tctatgtcga ctgggcaccc gaggctcctc tcccaccagc tctcttggtc agctgaaagc |
| 122161 | aaactgttaa caccctgggg agctggacgt atgagaccct tggggtggga ggcgttgatt |
| 122221 | tttgagagca atcacctggc cctggctggc agtaccggga cactgctgtg gctccggggt |
| 122281 | gggctgtctc cagaaaatgc ctggcctgag gcagccaccc gcatccagcc cagagggttt |
| 122341 | attcttgcaa tgtgctgctg cttcctgccc tgagcacctg gatcccggct tctgccctga |
| 122401 | ggccccttga gtcccacagg tagcaagcgc ttgccctgcg gctgctgcat ggggctaact |
| 122461 | aacgcttcct caccagtgtc tgctaagtgt ctcctctgtc tcccacgccc tgctctcctg |
| 122521 | tccccccagt ttgtctgctg tgaggggaca gaagaggtgt gtgccgcccc cacccctgcc |
| 122581 | cgggcccttg ttcctgggat tgctgttttc agctgtttga gctttgatcc tggttctctg |
| 122641 | gcttcctcaa agtgagctcg gccagaggag gaaggccatg tgctttctgg ttgaagtcaa |
| 122701 | gtctggtgcc ctggtggagg ctgtgctgct gaggcggagc tggggagaga gtgcacacgg |
| 122761 | gctgcgtggc caacccctct gggtagctga tgcccaaaga cgctgcagcg cccaggacat |
| 122821 | ctgggacctc cctggggccc gcccgtgtgt cccgcgctgt gttcatctgc gggctagcct |
| 122881 | gtgacccgcg ctgtgctcgt ctgcgggcta gcctgtgtcc cgcgctctgc ttgtctgcgg |
| 122941 | tctagcctgt gacctggcag agagccacca gatgtcccgg gctgagcact gccctctgag |
| 123001 | caccttcaca ggaagccctt ctcctggtga gaagagatgc cagcccctgg catctggggg |
| 123061 | cactggatcc ctggcctgag ccctagcctc tccccagcct gggggcccct tcccagcagg |
| 123121 | ctggccctgc tccttctcta cctgggaccc ttctgcctcc tggctggacc ctggaagctc |
| 123181 | tgcagggcct gctgtccccc tccctgccct ccaggtatcc tgaccaccgg ccctggctcc |
| 123241 | cactgccatc cactcctctc ctttctggcc gttccctggt ccctgtccca gcccccctcc |
| 123301 | ccctctcacg agttacctca cccaggccag agggaagagg gaaggaggcc ctggtcatac |
| 123361 | cagcacgtcc tcccacctcc ctcggccctg gtccaccccc tcagtgctgg cctcagagca |
| 123421 | cagctctctc caagccaggc cgcgcgccat ccatcctccc tgtcccccaa cgtccttgcc |
| 123481 | acagatcatg tccgccctga cacacatggg tcccagccat ctctgcccca gttaactccc |
| 123541 | catccataaa gagcacatgc cagctgacac caaaataatt cgggatggct ccagtttaga |
| 123601 | cctaagtgga aggagaaacc accacctgcc ctgcaccttg ttttttggtg accttgataa |
| 123661 | accatcttca gccatgaagc cagctgtctc ccaggaagct ccagggcggt gcttcctcgg |
| 123721 | gagctgactg ataggtggga ggtggctgcc cccttgcacc ctcaggtgac cccacacaag |
| 123781 | gccactgctg gaggccctgg ggactccagg aatgtcaatc agtgacctgc cccccaggcc |
| 123841 | ccacacagcc atggctgcat agaggcctgc ctccaaggga cctgtctgtc tgccactgtg |
| 123901 | gagtccctac agcgtgcccc ccacagggga gctggttctt tgactgagat cagctggcag |
| 123961 | ctcagggtca tcattcccag agggagcggt gccctggagg ccacaggcct cctcatgtgt |
| 124021 | gtctgcgtcc gctcgagctt actgagacac taaatctgtt ggtttctgct gtgccaccta |
| 124081 | cccaccctgt tggtgttgct ttgttcctat tgctaaagac aggaatgtcc aggacactga |
| 124141 | gtgtgcaggt gcctgctggt tctcacgtcc gagctgctga actccgctgg gtcctgctta |
| 124201 | ctgatggtct ttgctctagt gctttccagg gtccgtggaa gcttttcctg gaataaagcc |
| 124261 | cacgcatcga ccctcacagc gcctcccctc tttgaggccc agcagatacc ccactcctgc |
| 124321 | ctttccagca agatttttca gatgctgtgc atactcatca tattgatcac ttttttcttc |
| 124381 | atgcctgatt gtgatctgtc aatttcatgt caggaaaggg agtgacattt ttacacttaa |
| 124441 | gcgtttgctg agcaaatgtc tgggtcttgc acaatgacaa tgggtccctg tttttcccag |
| 124501 | aggctctttt gttctgcagg gattgaagac actccagtcc cacagtcccc agctcccctg |
| 124561 | gggcagggtt ggcagaattt cgacaacaca ttcttccacc ctgactagga tgtgctcctc |
| 124621 | atggcagctg ggaaccactg tccaataagg gcctgggctt acacagctgc ttctcattga |
| 124681 | gttacaccct taataaaata atcccatttt atcctttttg tctctctgtc ttcctctctc |
| 124741 | tctgcctttc ctcttctctc tcctcctctc tcatctccag gtgcaaatag tctacaaacc |
| 124801 | agttgacctg agcaaggtga cctccaagtg tggctcatta ggcaacatcc atcataaacc |
| 124861 | aggtagccct gtggaaggtg agggttggga cgggagggtg cagggggtgg aggagtcctg |
| 124921 | gtgaggctgg aactgctcca gacttcagaa ggggctggaa aggatattct aggtagacct |
| 124981 | acatcaagga aagtgttgag tgtgaaactt gcgggagccc aggaggcgtg gtggctccag |
| 125041 | ctcgctcctg cccaggccat gctgcccaag acaaggtgag gcgggagtga agtgaaataa |
| 125101 | ggcaggcaca gaaagaaagc acatattctc ggccgggcgc tgtggctcac gcctgtaatt |
| 125161 | ccagcacttt gggaggccaa ggtgggtgga tcatgaggtc aggagattga gaccatcctg |
| 125221 | gctaacacag tgaaaccccg tctctactaa aaatacaaaa aattagccgg gcgtggtggt |
| 125281 | gggcgcctgt agtcccagct actccggagg ctgaggcagg aaaatggcgt gaacccggaa |
| 125341 | ggcggagctt gcagtgagcg gagtgagcag agatcgcgcc actgcactcc agcctgggcg |
| 125401 | acagagcgag actccgtctc aaaaaaaaaa agcacatgtt ctcgcttctt tgtgggatcc |
| 125461 | aggagataga gaatagaagg atggttacca gaggctggga agggtagtga ggggatggtg |
| 125521 | gggggatggt caatgggtac aaaaaaaata gaataagacc tagtatttga tagtgcaaca |
| 125581 | gggtgactat agtcaataat aatttaattg tacatttaaa aataactaaa agatagccgg |
| 125641 | gtgcagtggc ttacgtctgt aatcccagta ctctgggagg ctgaggtggg cgtttgagac |
| 125701 | cagcctggcc aacatggtga aaccccatct ctactaaaaa tacaaaaatt agccaggcat |
| 125761 | ggtggcgggc gcctgtaatc ccagctactc gggaggctga ggcaggagaa tcacttgaac |
| 125821 | ctgggaggca gaggttgcag tgagccgaga tcttgccact gcactccagc ctgggtgaca |
| 125881 | gtgaaactcc gtctcaaaaa taaaaataaa aatacagctg ggcacggtgg ctcacgcctg |
| 125941 | taatcccagc actttgggag gccgaggcga gcggatcaca aggtcaggag atatagacca |
| 126001 | tcctggctaa cacggtgaaa cccggtctct actaaaaata caaaaaatta gccaggcgtg |
| 126061 | gtggcaggtg cctatagtcc cagctactca caaggctgag gcaggagaat ggcatgaacc |
| 126121 | tgggaggcgg agcttgcagt gagccgagat tgtgccactg cactccagcc tgggcgagag |
| 126181 | agtgagactc cgtctcaaaa caaaaacaaa aacaaaaaca aaaacaaaca cacaacaaaa |
| 126241 | acctaaaaga atataaatgg attgtttgta acacaaagga caaatgtttg aggggatgga |
| 126301 | taccccattt tccatgatgt gattattata cattgtgtgt ctgtatcaaa acatctcatg |
| 126361 | agccccataa atatatacac ctaactatgt acccacaaaa attaaaaaaa tatatttttt |
| 126421 | aaggtgaaga gggaggcgag atgctggcct taacccctaa cccgttgttc tccctgcaag |
| 126481 | ctgtccacag ggcctctcag actcgaggtt cagctatatg gatgcatgag cttggtcccc |
| 126541 | agccaacatg ggagacactt caccatcggc agcagctaca gcacaggaac cctgggtcac |
| 126601 | tgccatgtcc cctctgtgac tttgtttaaa cagaaaatga tgctctgggc cggctgtggt |
| 126661 | ggcccacacc tataatccca gcaccttggg aggcgggggt gggcagattg cctgaggtca |
| 126721 | ggagttggag atcagcctgg ccgacatggc gaaaccccat gtctactaaa aatacaaaaa |
| 126781 | ctagccaggc atggtggcac atgcctgtaa tcccagctac ttgggaggct gaagcaggag |
| 126841 | aatcacttga acccaggagg cagaggctga gtgagccaag atcgtgccaa tgcactccag |
| 126901 | cttgggtgag ggagtgagac tccgtctcaa aaaaaaaaaa aaagaaagaa aaagaaaaga |
| 126961 | aagtgatcct actggaacca tgcttactcc cctccccacc tcacactgtg tagaaattag |
| 127021 | tgctgtcggc caggcgcggt ggctcatgcc tgtaatcgca gcactttggg aggccaaggc |
| 127081 | aggcggatca cgaggtcagg agatcaagac catcctggct aacacagtga aaccctgtct |
| 127141 | ctactaaaaa tacaaaaaat tagccgggca tggtggcagg cacctgtagt cccaactact |
| 127201 | tgggaggctg aggcaggaga atggcatgaa cctgggaggc ggagcttgca gtgagccaag |
| 127261 | atcgcgccac tgcataccag cctaggtgac agagtgagac tcagcaaaaa aagaaagaaa |
| 127321 | gaaagaaaga aatcagtgct gtctatactt ctttctgcag tgatggaaat attctgtatc |
| 127381 | tgtgctgtcc agtatagtag ccactagcta catgtggcac ttgaaacatg gctggtacag |
| 127441 | ttgaggaaga gtggctgcca tatcggacga cacagctata gattctgtca ccccaccccg |
| 127501 | agagtccaga gcggggactt ctgccttagg ccctattcag ggctgatttt tacttgaacc |
| 127561 | cttactgtgg gaagagaagg ccatgagaag ttcagtctag aatgtgactc cttattttct |
| 127621 | ggctcccttg gacactttgt gggatttagt ctccctgtgg aaagtattcc acaagtggtg |
| 127681 | ccaccacccc agctgtgaga gcagctggga gctgcttttg tcatctttcc ctggaaagtc |
| 127741 | ctgtgggctg tctcttcctc atgccttgtc ccatgcttgg gcatggtgtc aagcgtcagg |
| 127801 | agggagaaag ggtccttatt tatttattta gagagggacc cttcttctgt tcccaggctg |
| 127861 | gagtgcagtg gtgcgatctc ggctcactgc aacctccgcc tcctgggttc aagtgattct |
| 127921 | cctgcctcag cctcctgagt agctgagatt acaggcacat gccaacatgc ctggctaatt |
| 127981 | tttttttttt tttttttttt ttttttttga gatggagttg tactctcatt gcccaggctg |
| 128041 | gaatgtaatg gcacaatctc ggctcactgc aacctccacc tcctggattc aagcaattct |
| 128101 | cctgtctcag cttcccaagt agctgggatt acaggtgccc gccaccatgc tcaactaatt |
| 128161 | tttgtatttt ttttttagta gagacgaggt ttcaccatgt tggtcagact ggtctcaaac |
| 128221 | tcctgacctc aggtgatcca cctgcctcgg cctcccaaag tgctaggatt acaggcatga |
| 128281 | gccaccacgc ccggcctgaa agggttctta tttagtgtgc attttgacat tcaatttaat |
| 128341 | tccaaggtct tgtggggtca tggtttacag gatgttgata tagaaaagac ttcacttaat |
| 128401 | gggccgggcg cagtggctca tgcctgtaat cccagcactt tgggaggccg aggcaggcag |
| 128461 | atcaggaggt caggagattg agaccatcct ggctaacaca gtgaaacccc atctctactg |
| 128521 | aaaatacaaa aaattagctg ggcgtggtgg caggcacctg tagtcccagc cactcggttg |
| 128581 | gctgaggcag gagaatggca tgaacccggg aggcggagct tgcagtgagc agagaccatg |
| 128641 | ccactgcact ccagcctggg cgacagagca agactctgtc tcaagaaaaa aaaaaaaaaa |
| 128701 | acagacttta cttactggaa gccaaccaat gtatatttag agtaatttct cctgggctga |
| 128761 | gctgtcattt acttttgcag tatctcaaga agaagagttt acagtgtaaa tatttgatgc |
| 128821 | acactttgat tatatagatg aagcaaacta ttttcaagag ctttgcaagg acttacttgt |
| 128881 | atccaaacac cattctaaag gagtcttacc tacttctaaa ggctggtctc tacttggaac |
| 128941 | cacttgcttg gccctggttc aagtcctgct gcaaacctgg aagtcctgtc attgtcttct |
| 129001 | tccctccaga gcagtggcac ccaatctaat ttctgctgtg ccccagcagc ccctggcact |
| 129061 | ttgccctgta gactgcagac ctcatgtaat gtatgttaag tccacagaac cacagaagat |
| 129121 | gatggcaaga tgctcttgtg tgtgttgtgt tctaggaggt ggccaggtgg aagtaaaatc |
| 129181 | tgagaagctt gacttcaagg acagagtcca gtcgaagatt gggtccctgg acaatatcac |
| 129241 | ccacgtccct ggcggaggaa ataaaaaggt aaagggggta gggtgggttg gatgctgccc |
| 129301 | ttgggtatat gggcattaat caagttgagt ggacaaaggc tggtccagct cccagaggag |
| 129361 | gaaaacagag gcttctgtgt tgactggctg gatgtgggcc ctcagcagca tccagtgggt |
| 129421 | ctccactgcc tgtctcaatc acctggagct ttagcacgtt tcacacctgg gccccaacct |
| 129481 | ggagaggctg accaatgggt ctcaggggca gctcggttgc tggagttttt gtttttattt |
| 129541 | atttttatgt atttaaggca gggtctctgt attagtccat tctcacactg ctaataaaga |
| 129601 | catacccaag actgggtaat ttataaagga aagaggttta atggactcac agttccacat |
| 129661 | ggctggggag gcctcaaaat catggcggaa ggcaaaggag aagcaaaggc atttcttaca |
| 129721 | tggcgacagg caagagagcg tgtgcagggg aactcccatt tataaaacca tcagacctca |
| 129781 | tgagatttat tcactatcat gagaacagca tgggaaagac ccgcccccat gattcagtta |
| 129841 | cctcccactg ggtccctccc atgacacatg gaattatggg agctacaatt caagatgaga |
| 129901 | tttgggtggg gacacagcca aaccatatca gtctccctct gtcatccagg ctggagtgca |
| 129961 | ctggcatgat ctcggctcac tgcagcctct acctccctgg gtcaggtgat cttcccacct |
| 130021 | cagcctccca ggtagctgga actacaggta cctgccacta tgcctggcta aatattttgt |
| 130081 | atttcctgtg gagacgaggt tttgccacgt tgcccaggct ggtcttgaac tcctgaggtc |
| 130141 | aagcaatatg cccacctcgg cctcccaagg tgctgggatt acaggtgtga gccacagtgc |
| 130201 | tcggcctaag tcactgcagt ttttaaagct cccaggtgat tcttcagtgc agtcaaaagt |
| 130261 | gagaactggc tgggtgcggt ggctcatgcc tgtaatccca gcaccttggg aggcgaaggt |
| 130321 | gggcagatgg cttgaggtca ggagttcaag accagcctgg ccaacatggt aaaaccccat |
| 130381 | ctctactaaa aatacaaaag ttagctgggt gtggtggtgc gtgcctgtaa tcccagctac |
| 130441 | ttgggaggct gaggcatgag aattgcttga acccagggga cagaggttgt agtgagccga |
| 130501 | gatcgtgcca ctgcactcca gcctgggcaa cagagtgaga ttccatctca caaaaaaaaa |
| 130561 | aaaaaaagcg agaaccactg tcctaggccc tgatgtttgc aggcaactaa aaaaggaagt |
| 130621 | ggacatcccc agtcagctgt ggcgcaccaa gaacaagtca tgggaacata acctaatttt |
| 130681 | ctaaatgggt tactaggcac ttagagcaaa acaatgatgc cgaaatcctg atttcagcaa |
| 130741 | agcctctgcc tgcctgtctt ggaagtatcc acatgaggct gctggggcct tggtgtcccc |
| 130801 | agcagtttct agtctctagg tcttgctgtg ggtgtctgtg cagtgagggt gtgtgtggcg |
| 130861 | ctgggtgagc tctgtctagg cctggcacag gatgcggtct ggtagctgct gcttctcttc |
| 130921 | tgcagaagcg cagccaagca ccctctgggg tttcaggccc acacccagcc tgaagttctg |
| 130981 | ggagtggctc actttccaac cttcagggtc tcccagcagc tgactgggga gtggtggagg |
| 131041 | gaaaagggat tgtattagtc cgttttcacg ccgctgatga agacataccc gatactgggc |
| 131101 | agtctaaaag atagaggtct gatggactca cagttccacg tgactgggga ggcctgacaa |
| 131161 | tcatggtgga aggtgaaagg cttgtctcac acggtggcag acaagagaaa agagcttgtg |
| 131221 | caggggaact cccctttata aaaccatcag atctcgggag acttattcac tatcatgaga |
| 131281 | acagcacggg aaagaccctc ctctatgatt caattacctc ccaccaggtc cctcccacaa |
| 131341 | catgtaggaa ttgtgggaac tacaattcaa gatgacattt gggtggggac acagccaaac |
| 131401 | catatcaggg cgtcccagaa agggtatagg gtctgagacc caagtcagca tgagaaagta |
| 131461 | tgcttctcat ggtggcccag ttgggtggaa gtggcagccg ggccgtcttt ccaccaggcc |
| 131521 | actcaagtag cagctgagag acccctgccc tggccagtcc ccgccctccc ctcttgccac |
| 131581 | tgcctctggt tctgaacaga tgggcaccct catcttgtat ttgtgattaa tgtctaacaa |
| 131641 | tgtagttttg tgagaagggt ttgctgatac agccttgctg cagatgctgc gaactgtggc |
| 131701 | ctggggcaga ccttacctcc agacacgccc tgaggcaggg gagggcactg gcccgtagct |
| 131761 | ggccgagagc tctcgggttg cgcgacaggg atacttttca gcggctgggt cgctatccaa |
| 131821 | agtgagaaaa cgaggaggga ccaggaggct gtccgcctca agagatgtgg gggccaggtc |
| 131881 | cagttatctg gggaagcagt aagcttctct gctgtttcta accccaggcc tcccctggtc |
| 131941 | taaggcaggg cctcccagcc tcggggcact ttaaagatat ctgggcctgg ccccatcccc |
| 132001 | acagtctgac tgagtgggtc tggatagggc ctgagcattg gtgatttcct gggtgaaagg |
| 132061 | aggcccctca cagtctctgg aagcttctct gtgttaggaa aagctctggg cttgactctg |
| 132121 | ctttgaaagt caagatccgc aaatcctctc agcctcagtt tctccttcag caagatgaaa |
| 132181 | tggaaatgct gtacctacgt cccggggtgg ttgtgagacc caaaaaagac aatgttctgg |
| 132241 | aaggttcctg gtgcgttgca gtcctctaag aacctgagtt agagccacgc tgagtctcag |
| 132301 | cttcttggct ccttctgttt caaactcgtc catgtgatag ctcaggaagg gtaggcaggg |
| 132361 | ccctgccccc tactcagaaa acaccatcct ggtcctgggg atccccgcag cattagtccc |
| 132421 | ctgttttccc agtgtattga gaaaaattgc taacaagcag tggggcacac caccagcctc |
| 132481 | ctgggttcct ttcagtttgg ggatttttgg acattcccag gaatgtctta aaaaacactt |
| 132541 | caaaaaacat taacataaat atttttatca aagcctgtat taaatggtct ttcaagaaaa |
| 132601 | tacagtaaca ggtcaggcat ggtggctcat gcctgtaacc ccagcactct gggaggccaa |
| 132661 | ggcaggcaga tcacctgaaa tcaggagttc aagaccaacc tggccaacac agccaaatcc |
| 132721 | catctctaca aaaaatacaa aaattagctg ggtgtggtgg cacacacctg tagtcccagc |
| 132781 | tacttgggag gccgaggcag gagaattgct tgatcccgga ggcggaggct gcagtgagct |
| 132841 | gagatcgtgc cactgcactc cagcgtgggt gacaaggtga atctttgtct caaaaaaaaa |
| 132901 | aaaaaaaaaa agataaaata cagtatacag taatagagaa caatcctttt ttcaaagtag |
| 132961 | tgaccccaaa tgaacaaaat atgcatctag cttaaatgcg aacctggttt tctctacgcc |
| 133021 | cattcaagcc cctgcaatag gggcccttca ccccgcatcc atggactcct aaaattatat |
| 133081 | ggaaaatggc tgtgtgtgag tgtggatgga catgtgcaca catattttcg gctttaccag |
| 133141 | atgctcaaag agcctaggac ccaaaaaggg ctgagaatga ccgtgtcggc cacttcaggg |
| 133201 | tcatcaggaa ttgctgtgca ctgctcactt ctccagtgaa cactttctgc ttctgtgttt |
| 133261 | cctggtatcc tttgggactc ctggctaggt catgtgtttc tctactttca aaagggcttc |
| 133321 | agccaggcac gatggcatga gcctgtagtc ccagttgctc tggaggttaa ggtgggaaga |
| 133381 | ttgcttgagc ccaggaattt gaggccagcc tgggcaagta gataggtaga tgattgatag |
| 133441 | atagatagat agataaatag atggatagat aagtcgctag acagtcatcc atccacccat |
| 133501 | ccacacataa aaaggccttt gtcatgtcat gtcttgtggc ccacctgcca gtgttgccca |
| 133561 | cagttgctgc ccctccaaac tcatcagtca ctggcaaaca ggaggaatgt gtggctcatg |
| 133621 | tctgggcatc agtggctgtg ggagacatcc ttgatcttct ccagcttctc cttccacatt |
| 133681 | ttcctttgca atctggcaat atctattaaa ataaaatgtg catgcctttt gacctaagag |
| 133741 | cttcacttct aggacccact tacacgtgtg tgacatgatg ttcatacggg tttatttatc |
| 133801 | tgaggttgtt catacacacc attgcctgta atcactaaag gcgggagcag cctacacatc |
| 133861 | catccacaga ggagtagatg ccttttggta catccgtggc gacggaatac taagcagcct |
| 133921 | gtgtatctat acactcacac gtgtttgttt atgtgtggaa tatctctgga gggtacacaa |
| 133981 | gaaacttaaa atgatcactg tctctgggga gggtacctgg gtgcctggga ggcaggtcag |
| 134041 | ggaaggagtg ggcacaggta ttaccaattg gaagacaata aaaacaacag ctcctggcca |
| 134101 | ggcgcagtgg ctcacgcctg taatggcagc actctgagag gctgaggcgg gcagattgct |
| 134161 | tgcgtccagg agttcaagac cagcctgggc aacatagcaa aaccccgttt ctattaaaaa |
| 134221 | tacaaaaaat tagccaggtg tggtggcatg cacctgtaat cccagctact cgggaggctg |
| 134281 | aggtgggaga atcacctgag cctgggaggt caaggctgca gtgaggtgag attgtgccac |
| 134341 | cgcactctag cctgggcgat agagcaagac cctgtctcaa aaacaaacaa aaaacagtcc |
| 134401 | ctggcactct gggccaggcc tggcagggca gttggcaggg ctggtctttc tctggcactt |
| 134461 | catctcaccc tccctccctt cctcttcttg cagattgaaa cccacaagct gaccttccgc |
| 134521 | gagaacgcca aagccaagac agaccacggg gcggagatcg tgtacaagtc gccagtggtg |
| 134581 | tctggggaca cgtctccacg gcatctcagc aatgtctcct ccaccggcag catcgacatg |
| 134641 | gtagactcgc cccagctcgc cacgctagct gacgaggtgt ctgcctccct ggccaagcag |
| 134701 | ggtttgtgat caggcccctg gggcggtcaa taattgtgga gaggagagaa tgagagagtg |
| 134761 | tggaaaaaaa aagaataatg acccggcccc cgccctctgc ccccagctgc tcctcgcagt |
| 134821 | tcggttaatt ggttaatcac ttaacctgct tttgtcactc ggctttggct cgggacttca |
| 134881 | aaatcagtga tgggagtaag agcaaatttc atctttccaa attgatgggt gggctagtaa |
| 134941 | taaaatattt aaaaaaaaac attcaaaaac atggccacat ccaacatttc ctcaggcaat |
| 135001 | tccttttgat tcttttttct tccccctcca tgtagaagag ggagaaggag aggctctgaa |
| 135061 | agctgcttct gggggatttc aagggactgg gggtgccaac cacctctggc cctgttgtgg |
| 135121 | gggtgtcaca gaggcagtgg cagcaacaaa ggatttgaaa cttggtgtgt tcgtggagcc |
| 135181 | acaggcagac gatgtcaacc ttgtgtgagt gtgacggggg ttggggtggg gcgggaggcc |
| 135241 | acgggggagg ccgaggcagg ggctgggcag aggggagagg aagcacaaga agtgggagtg |
| 135301 | ggagaggaag ccacgtgctg gagagtagac atccccctcc ttgccgctgg gagagccaag |
| 135361 | gcctatgcca cctgcagcgt ctgagcggcc gcctgtcctt ggtggccggg ggtgggggcc |
| 135421 | tgctgtgggt cagtgtgcca ccctctgcag ggcagcctgt gggagaaggg acagcgggta |
| 135481 | aaaagagaag gcaagctggc aggagggtgg cacttcgtgg atgacctcct tagaaaagac |
| 135541 | tgaccttgat gtcttgagag cgctggcctc ttcctccctc cctgcagggt agggggcctg |
| 135601 | agttgagggg cttccctctg ctccacagaa accctgtttt attgagttct gaaggttgga |
| 135661 | actgctgcca tgattttggc cactttgcag acctgggact ttagggctaa ccagttctct |
| 135721 | ttgtaaggac ttgtgcctct tgggagacgt ccacccgttt ccaagcctgg gccactggca |
| 135781 | tctctggagt gtgtgggggt ctgggaggca ggtcccgagc cccctgtcct tcccacggcc |
| 135841 | actgcagtca ccccgtctgc gccgctgtgc tgctgtctgc cgtgagagcc caatcactgc |
| 135901 | ctatacccct catcacacgt cacaatgtcc cgaattccca gcctcaccac cccttctcag |
| 135961 | taatgaccct ggttggttgc aggaggtacc tactccatac tgagggtgaa attaagggaa |
| 136021 | ggcaaagtcc aggcacaaga gtgggacccc agcctctcac tctcagttcc actcatccaa |
| 136081 | ctgggaccct caccacgaat ctcatgatct gattcggttc cctgtctcct cctcccgtca |
| 136141 | cagatgtgag ccagggcact gctcagctgt gaccctaggt gtttctgcct tgttgacatg |
| 136201 | gagagagccc tttcccctga gaaggcctgg ccccttcctg tgctgagccc acagcagcag |
| 136261 | gctgggtgtc ttggttgtca gtggtggcac caggatggaa gggcaaggca cccagggcag |
| 136321 | gcccacagtc ccgctgtccc ccacttgcac cctagcttgt agctgccaac ctcccagaca |
| 136381 | gcccagcccg ctgctcagct ccacatgcat agtatcagcc ctccacaccc gacaaagggg |
| 136441 | aacacacccc cttggaaatg gttcttttcc cccagtccca gctggaagcc atgctgtctg |
| 136501 | ttctgctgga gcagctgaac atatacatag atgttgccct gccctcccca tctgcaccct |
| 136561 | gttgagttgt agttggattt gtctgtttat gcttggattc accagagtga ctatgatagt |
| 136621 | gaaaagaaaa aaaaaaaaaa aaaaggacgc atgtatcttg aaatgcttgt aaagaggttt |
| 136681 | ctaacccacc ctcacgaggt gtctctcacc cccacactgg gactcgtgtg gcctgtgtgg |
| 136741 | tgccaccctg ctggggcctc ccaagttttg aaaggctttc ctcagcacct gggacccaac |
| 136801 | agagaccagc ttctagcagc taaggaggcc gttcagctgt gacgaaggcc tgaagcacag |
| 136861 | gattaggact gaagcgatga tgtccccttc cctacttccc cttggggctc cctgtgtcag |
| 136921 | ggcacagact aggtcttgtg gctggtctgg cttgcggcgc gaggatggct ctctctggtc |
| 136981 | atagcccgaa gtctcatggc agtcccaaag gaggcttaca actcctgcat cacaagaaaa |
| 137041 | aggaagccac tgccagctgg ggggatctgc agctcccaga agctccgtga gcctcagcca |
| 137101 | cccctcagac tgggttcctc tccaagctcg ccctctggag gggcagcgca gcctcccacc |
| 137161 | aagggccctg cgaccacagc agggattggg atgaattgcc tgtcctggat ctgctctaga |
| 137221 | ggcccaagct gcctgcctga ggaaggatga cttgacaagt caggagacac tgttcccaaa |
| 137281 | gccttgacca gagcacctca gcccgctgac cttgcacaaa ctccatctgc tgccatgaga |
| 137341 | aaagggaagc cgcctttgca aaacattgct gcctaaagaa actcagcagc ctcaggccca |
| 137401 | attctgccac ttctggtttg ggtacagtta aaggcaaccc tgagggacct ggcagtagaa |
| 137461 | atccagggcc tcccctgggg ctggcagctt cgtgtgcagc tagagcttta cctgaaagga |
| 137521 | agtctctggg cccagaactc tccaccaaga gcctccctgc cgttcgctga gtcccagcaa |
| 137581 | ttctcctaag ttgaagggat ctgagaagga gaaggaaatg tggggtagat ttggtggtgg |
| 137641 | ttagagatat gcccccctca ttactgccaa cagtttcggc tgcatttctt cacgcacctc |
| 137701 | ggttcctctt cctgaagttc ttgtgccctg ctcttcagca ccatgggcct tcttatacgg |
| 137761 | aaggctctgg gatctccccc ttgtggggca ggctcttggg gccagcctaa gatcatggtt |
| 137821 | tagggtgatc agtgctggca gataaattga aaaggcacgc tggcttgtga tcttaaatga |
| 137881 | ggacaatccc cccagggctg ggcactcctc ccctcccctc acttctccca cctgcagagc |
| 137941 | cagtgtcctt gggtgggcta gataggatat actgtatgcc ggctccttca agctgctgac |
| 138001 | tcactttatc aatagttcca tttaaattga cttcagtggt gagactgtat cctgtttgct |
| 138061 | attgcttgtt gtgctatggg gggagggggg aggaatgtgt aagatagtta acatgggcaa |
| 138121 | agggagatct tggggtgcag cacttaaact gcctcgtaac ccttttcatg atttcaacca |
| 138181 | catttgctag agggagggag cagccacgga gttagaggcc cttggggttt ctcttttcca |
| 138241 | ctgacaggct ttcccaggca gctggctagt tcattccctc cccagccagg tgcaggcgta |
| 138301 | ggaatatgga catctggttg ctttggcctg ctgccctctt tcaggggtcc taagcccaca |
| 138361 | atcatgcctc cctaagacct tggcatcctt ccctctaagc cgttggcacc tctgtgccac |
| 138421 | ctctcacact ggctccagac acacagcctg tgcttttgga gctgagatca ctcgcttcac |
| 138481 | cctcctcatc tttgttctcc aagtaaagcc acgaggtcgg ggcgagggca gaggtgatca |
| 138541 | cctgcgtgtc ccatctacag acctgcagct tcataaaact tctgatttct cttcagcttt |
| 138601 | gaaaagggtt accctgggca ctggcctaga gcctcacctc ctaatagact tagccccatg |
| 138661 | agtttgccat gttgagcagg actatttctg gcacttgcaa gtcccatgat ttcttcggta |
| 138721 | attctgaggg tggggggagg gacatgaaat catcttagct tagctttctg tctgtgaatg |
| 138781 | tctatatagt gtattgtgtg ttttaacaaa tgatttacac tgactgttgc tgtaaaagtg |
| 138841 | aatttggaaa taaagttatt actctgatta aataaggtct ccattcatgg attccaagga |
| 138901 | caagaaagtc atatagaatg tctatttttt aagttctttc ccacgcaccc ttagataatt |
| 138961 | tagctcagaa caggaaatga tagtattaat aaaagctgga catcaggatt aacagctctc |
| 139021 | tctggggccc tgaaggtgag agttctcaga cttgctcatt tgcagttgct tctttgtgat |
| 139081 | gctggcaaac catcctagtc ccattcaaag ggcaatacaa agccttgtgg ctgacctcac |
| 139141 | gatgcagcac tcagtttgca agaccggcac cagtgtatgc aaacctgaga aggttgggga |
| 139201 | tgaggatatg ggatctttca tccctggaaa tttagtccag aggcctgggg ctggagcaga |
| 139261 | acaccaagcc aatcagctta atgaatggct tagattcctg ctaggtttgc agagctgcct |
| 139321 | tctttccttt ggtaccttat tatagattga ggagtatttc tgctaaacca agatagggat |
| 139381 | aaccagatag catcttcata gcaatgccac aaaggaaaac aaaaacaaaa cagtaatcca |
| 139441 | tcatattatt ccttagtaac tatgccaagg tcatgatact gaatccttag attgtttcaa |
| 139501 | aatactactt ttctttgctc ttcctgatgt gtttgccacc gcaggcagat gtttaagtaa |
| 139561 | aacagatttt aactgcagct acaaaagcag caacaggcca gcaaaagaga agtgctatct |
| 139621 | cagagagcat ggctttcaga gccacaagag acagcctcac tggctgtttc agcttgactg |
| 139681 | ccatgcaaag aagagagcag agggagaacc agccccaccc acttattcat cttgtacaaa |
| 139741 | aaaaaagcac ctaccagcct aggctacata gtgagacact atctccacaa aaaacccacg |
| 139801 | aaaactagct gggtatggtg gcacatgcct acagtcccag ctactggtaa ggctgtggtg |
| 139861 | ggaggatctc ttgaggccag gaaggagatc caggctgcag tgagccaaga ttgcaccact |
| 139921 | gcactccagt ctggacaatc gagcaagatc ccatctcaaa caataaaaaa aaaaagcgtg |
| 139981 | taacctcctc agaagaaaga tgttataatc tcaggcagca ggcaagaacc aatccaggct |
| 140041 | ctaagcaaat tatgtatctc actgacccca ccaaacctca gaaaaattta acagtgagaa |
| 140101 | gcaaaatctc ctttaaagag caacttagaa cagatagaaa atatcataca gctgacttca |
| 140161 | ctagagagaa agtgcatcaa ctgctttcac tcaacaaaaa gaaaaaagag atgatcaatg |
| 140221 | cagatcccct ctcctcctgg cagcccttac cctcagtgaa aagccaccac cattctctct |
| 140281 | ctggtggcca tcagatcaac ctgcggcgtt cccacaagac agaatggaga ttttccaagg |
| 140341 | tatagagcaa gtcagagtac cccaaagaac ggcggcagag agccagctcc gaaactgcca |
| 140401 | acactaccat gcatacacag ttcagtaagt caagaaaggc ctggtacaca gcattctgta |
| 140461 | actttttttt ttattttttt caatttttcc ttcttttttt tttttaagca ctagtctgtg |
| 140521 | ctttgcgaac agaatcaaga cattaacaaa gatcagcttc tctgaagaaa agcatttcta |
| 140581 | tagaacaaag acagctacat gtttcgctgc cattacacag ctccaaagca ggaaaagaaa |
| 140641 | atatttacaa aatacaaggt tttttttttc cattttttgt ttttgttttt tttttcaatg |
| 140701 | ctaaaagggt tattcagaat tttcaacctt ataaatagaa gaagcactct atgcataggg |
| 140761 | atatggtgca ttattgtatt tttttttaaa gaaacaatga caaacccttt aacttgcaaa |
| 140821 | cagaaaaaaa aatcactaat gttgaaaatt gtgaaaaaac cccaaccatt aa |

In various embodiments, the MAPT gene product is a MAPT mRNA sequence comprising NCBI Reference Sequence NM_001123066.4 (SEQ ID NO: 301569). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001123066.4 (SEQ ID NO: 301569).

| (SEQ ID NO: 301569) | |
|---|---|
| 1 | gcagtcaccg ccacccacca gctccggcac caacagcagc gccgctgcca ccgcccacct |
| 61 | tctgccgccg ccaccacagc caccttctcc tcctccgctg tcctctcccg tcctcgcctc |
| 121 | tgtcgactat caggtgaact ttgaaccagg atggctgagc cccgccagga gttcgaagtg |
| 181 | atggaagatc acgctgggac gtacgggttg ggggacagga aagatcaggg gggctacacc |
| 241 | atgcaccaag accaagaggg tgacacggac gctggcctga aagaatctcc cctgcagacc |
| 301 | cccactgagg acggatctga ggaaccgggc tctgaaacct ctgatgctaa gagcactcca |
| 361 | acagcggaag atgtgacagc acccttagtg gatgagggag ctcccggcaa gcaggctgcc |
| 421 | gcgcagcccc acacggagat cccagaagga accacagctg aagaagcagg cattggagac |
| 481 | acccccagcc tggaagacga agctgctggt cacgtgaccc aagagcctga aagtggtaag |
| 541 | gtggtccagg aaggcttcct ccgagagcca ggccccccag gtctgagcca ccagctcatg |
| 601 | tccggcatgc ctggggctcc cctcctgcct gagggcccca gagaggccac acgccaacct |
| 661 | tcggggacag gacctgagga cacagagggc ggccgccacg cccctgagct gctcaagcac |
| 721 | cagcttctag gagacctgca ccaggagggg ccgccgctga agggggcagg gggcaaagag |
| 781 | aggccgggga gcaaggagga ggtggatgaa gaccgcgacg tcgatgagtc ctccccccaa |
| 841 | gactcccctc cctccaaggc ctccccagcc caagatgggc ggcctcccca gacagccgcc |
| 901 | agagaagcca ccagcatccc aggcttccca gcggagggtg ccatccccct ccctgtggat |
| 961 | ttcctctcca aagtttccac agagatccca gcctcagagc ccgacgggcc cagtgtaggg |
| 1021 | cgggccaaag ggcaggatgc ccccctggag ttcacgtttc acgtggaaat cacacccaac |
| 1081 | gtgcagaagg agcaggcgca ctcggaggag catttgggaa gggctgcast tccaggggcc |
| 1141 | cctggagagg ggccagaggc ccggggcccc tctttgggag aggacacaaa agaggctgac |
| 1201 | cttccagagc cctctgaaaa gcagcctgct gctgctccgc gggggaagcc cgtcagccgg |
| 1261 | gtccctcaac tcaaagctcg catggtcagt aaaagcaaag acgggactgg aagcgatgac |
| 1321 | aaaaaagcca agacatccac acgttcctct gctaaaacct tgaaaaatag gccttgcctt |
| 1381 | agccccaaac accccactcc tggtagctca gaccctctga tccaaccctc cagccctgct |
| 1441 | gtgtgcccag agccaccttc ctctcctaaa tacgtctctt ctgtcactic ccgaactggc |
| 1501 | agttctggag caaaggagat gaaactcaag ggggctgatg gtaaaacgaa gatcgccaca |
| 1561 | ccgcggggag cagcccctcc aggccagaag ggccaggcca acgccaccag gattccagca |
| 1621 | aaaaccccgc ccgctccaaa gacaccaccc agctctgcga ctaagcaagt ccagagaaga |
| 1681 | ccaccccctg cagggcccag atctgagaga ggtgaacctc caaaatcagg ggatcgcagc |
| 1741 | ggctacagca gccccggctc cccaggcact cccggcagcc gctcccgcac cccgtccctt |
| 1801 | ccaaccccac ccacccggga gcccaagaag gtggcagtgg tccgtactcc acccaagtcg |
| 1861 | ccgtcttccg ccaagagccg cctgcagaca gcccccgtgc ccatgccaga cctgaagaat |
| 1921 | gtcaagtcca agatcggctc cactgagaac ctgaagcacc agccgggagg cgggaaggtg |
| 1981 | cagataatta ataagaagct ggatcttagc aacgtccagt ccaagtgtgg ctcaaaggat |
| 2041 | aatatcaaac acgtcccggg aggcggcagt gtgcaaatag tctacaaacc agttgacctg |
| 2101 | agcaaggtga cctccaagtg tggctcatta ggcaacatcc atcataaacc aggaggtggc |
| 2161 | caggtggaag taaaatctga gaagcttgac ttcaaggaca gagtccagic gaagattggg |
| 2221 | tccctggaca atatcaccca cgtccctggc ggaggaaata aaaagattga aacccacaag |
| 2281 | ctgaccttcc gcgagaacgc caaagccaag acagaccacg gggcggagat cgtgtacaag |
| 2341 | tcgccagtgg tgtctgggga cacgtctcca cggcatctca gcaatgtctc ctccaccggc |
| 2401 | agcatcgaca tggtagactc gccccagctc gccacgctag ctgacgaggt gtctgcctcc |
| 2461 | ctggccaagc agggtttgtg atcaggcccc tggggcggtc aataattgtg gagaggagag |
| 2521 | aatgagagag tgtggaaaaa aaaagaataa tgacccggcc cccgccctct gcccccagct |
| 2581 | gctcctcgca gttcggttaa ttggttaatc acttaacctg cttttgtcac tcggctttgg |
| 2641 | ctcgggactt caaaatcagt gatgggagta agagcaaatt tcatctttcc aaattgatgg |
| 2701 | gtgggctagt aataaaatat ttaaaaaaaa acattcaaaa acatggccac atccaacatt |
| 2761 | tcctcaggca attccttttg attctttttt cttccccctc catgtagaag agggagaagg |
| 2821 | agaggctctg aaagctgctt ctgggggatt tcaagggact gggggtgcca accacctctg |
| 2881 | gccctgttgt gggggtgtca cagaggcagt ggcagcaaca aaggatttga aacttggtgt |
| 2941 | gttcgtggag ccacaggcag acgatgtcaa ccttgtgtga gtgtgacggg ggttggggtg |
| 3001 | gggcgggagg ccacggggga ggccgaggca ggggctgggc agaggggaga ggaagcacaa |
| 3061 | gaagtgggag tgggagagga agccacgtgc tggagagtag acatccccct ccttgccgct |
| 3121 | gggagagcca aggcctatgc cacctgcagc gtctgagcgg ccgcctgtcc ttggtggccg |
| 3181 | ggggtggggg cctgctgtgg gtcagtgtgc caccctctgc agggcagcct gtgggagaag |
| 3241 | ggacagcggg taaaaagaga aggcaagctg gcaggagggt ggcacttcgt ggatgacctc |
| 3301 | cttagaaaag actgaccttg atgtcttgag agcgctggcc tcttcctccc tccctgcagg |
| 3361 | gtagggggcc tgagttgagg ggcttccctc tgctccacag aaaccctgst ttattgagtt |
| 3421 | ctgaaggttg gaactgctgc catgattttg gccactttgc agacctggga ctttagggct |
| 3481 | aaccagttct ctttgtaagg acttgtgcct ct-gggagac gtccacccgt ttccaagcct |
| 3541 | gggccactgg catctctgga gtgtgtgggg gtctgggagg caggtcccga gccccctgtc |
| 3601 | cttcccacgg ccactgcagt caccccgtct gcgccgctgt gctgttgtct gccgtgagag |
| 3661 | cccaatcact gcctataccc ctcatcacac gtcacaatgt cccgaattcc cagcctcacc |
| 3721 | accccttctc agtaatgacc ctggttggtt gcaggaggta cctactccat actgagggtg |
| 3781 | aaattaaggg aaggcaaagt ccaggcacaa gagtgggacc ccagcctcsc actctcagtt |
| 3841 | ccactcatcc aactgggacc ctcaccacga atctcatgat ctgattcggt tccctgtctc |
| 3901 | ctcctcccgt cacagatgtg agccagggca ctgctcagct gtgaccctag gtgtttctgc |
| 3961 | cttgttgaca tggagagagc cctttcccct gagaaggcct ggccccttcc tgtgctgagc |
| 4021 | ccacagcagc aggctgggtg tcttggttgt cagtggtggc accaggatgg aagggcaagg |
| 4081 | cacccagggc aggcccacag tcccgctgtc ccccacttgc accctagcst gtagctgcca |
| 4141 | acctcccaga cagcccagcc cgctgctcag ctccacatgc atagtatcag ccctccacac |
| 4201 | ccgacaaagg ggaacacacc cccttggaaa tggttctttt cccccagtcc cagctggaag |
| 4261 | ccatgctgtc tgttctgctg gagcagctga acatatacat agatgttgcc ctgccctccc |
| 4321 | catctgcacc ctgttgagtt gtagttggat ttgtctgttt atgcttggat tcaccagagt |
| 4381 | gactatgata gtgaaaagaa aaaaaaaaaa aaaaaaggac gcatgtatct tgaaatgctt |
| 4441 | gtaaagaggt ttctaaccca ccctcacgag gtgtctctca cccccacact gggactcgtg |
| 4501 | tggcctgtgt ggtgccaccc tgctggggcc tcccaagttt tgaaaggcst tcctcagcac |
| 4561 | ctgggaccca acagagacca gcttctagca gctaaggagg ccgttcagct gtgacgaagg |
| 4621 | cctgaagcac aggattagga ctgaagcgat gatgtcccct tccctactic cccttggggc |
| 4681 | tccctgtgtc agggcacaga ctaggtcttg tggctggtct ggcttgcggc gcgaggatgg |
| 4741 | ttctctctgg tcatagcccg aagtctcatg gcagtcccaa aggaggctta caactcctgc |
| 4801 | atcacaagaa aaaggaagcc actgccagct ggggggatct gcagctccca gaagctccgt |
| 4861 | gagcctcagc cacccctcag actgggttcc tcrccaagct cgccctctgg aggggcagcg |
| 4921 | cagcctccca ccaagggccc tgcgaccaca gcagggattg ggatgaattg cctgtcctgg |
| 4981 | atctgctcta gaggcccaag ctgcctgcct gaggaaggat gacttgacaa gtcaggagac |
| 5041 | actgttccca aagccttgac cagagcacct cagcccgctg accttgcaca aactccatct |
| 5101 | gctgccatga gaaaagggaa gccgcctttg caaaacattg ctgcctaaag aaactcagca |
| 5161 | gcctcaggcc caattctgcc acttctggtt tgggtacagt taaaggcaac cctgagggac |
| 5221 | ttggcagtag aaatccaggg cctcccctgg ggctggcagc ttcgtgtgca gctagagctt |
| 5281 | tacctgaaag gaagtctctg ggcccagaac tctccaccaa gagcctccct gccgttcgct |
| 5341 | gagtcccagc aattctccta agttgaaggg atctgagaag gagaaggaaa tgtggggtag |
| 5401 | atttggtggt ggttagagat atgcccccct cattactgcc aacagtttcg gctgcatttc |
| 5461 | ttcacgcacc tcggttcctc ttcctgaagt tcttgtgccc tgctcttcag caccatgggc |
| 5521 | cttcttatac ggaaggctct gggatctccc ccttgtgggg caggctcttg gggccagcct |
| 5581 | aagatcatgg tttagggtga tcagtgctgg cagataaatt gaaaaggcac gctggcttgt |
| 5641 | gatcttaaat gaggacaatc cccccagggc tgggcactcc tcccctcccc tcacttctcc |
| 5701 | cacctgcaga gccagtgtcc ttgggtgggc tagataggat atactgtatg ccggctcctt |
| 5761 | caagctgctg actcacttta tcaatagttc catttaaatt gacttcagcg gtgagactgt |
| 5821 | atcctgtttg ctattgcttg ttgtgctatg gggggagggg ggaggaatgt gtaagatagt |
| 5881 | taacatgggc aaagggagat cttggggtgc agcacttaaa ctgcctcgta acccttttca |
| 5941 | tgatttcaac cacatttgct agagggaggg agcagccacg gagttagagg cccttggggt |
| 6001 | ttctcttttc cactgacagg ctttcccagg cagctggcta gttcattccc tccccagcca |
| 6061 | ggtgcaggcg taggaatatg gacatctggt tgctttggcc tgctgccccc tttcaggggt |
| 6121 | cctaagccca caatcatgcc tccctaagac cttggcatcc ttccctctaa gccgttggca |
| 6181 | cctctgtgcc acctctcaca ctggctccag acacacagcc tgtgcttttg gagctgagat |
| 6241 | cactcgcttc accctcctca tctttgttct ccaagtaaag ccacgaggtc ggggcgaggg |
| 6301 | cagaggtgat cacctgcgtg tcccatctac agacctgcag cttcataaaa cttctgattt |
| 6361 | ctcttcagct ttgaaaaggg ttaccctggg cactggccta gagcctcacc tcctaataga |
| 6421 | cttagcccca tgagtttgcc atgttgagca ggactatttc tggcacttgc aagtcccatg |
| 6481 | atttcttcgg taattctgag ggtgggggga gggacatgaa atcatcttag cttagctttc |
| 6541 | tgtctgtgaa tgtctatata gtgtattgtg tgctttaaca aatgatttac actgactgtt |
| 6601 | gctgtaaaag tgaatttgga aataaagtta ttactctgat taaa. |
| | NCBI Reference Sequence NM_001123066.4 |

In various embodiments, the MAPT gene product is a MAPT mRNA sequence comprising NCBI Reference Sequence NM_001123067.4 (SEQ ID NO: 301570). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001123067.4 (SEQ ID NO: 301570).

In various embodiments, the MAPT gene product is a MAPT mRNA sequence comprising NCBI Reference Sequence NM_001203251.2 (SEQ ID NO: 301571). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001203251.2 (SEQ ID NO: 301571).

In various embodiments, the MAPT gene product is a MAPT mRNA sequence comprising NCBI Reference Sequence NM_001203252.2 (SEQ ID NO: 301572). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001203252.2 (SEQ ID NO: 301572).

In various embodiments, the MAPT gene product is a MAPT mRNA sequence comprising NCBI Reference Sequence NM_001377265.1 (SEQ ID NO: 301573). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001377265.1 (SEQ ID NO: 301573).

In various embodiments, the MAPT gene product is a MAPT mRNA sequence comprising NCBI Reference Sequence NM_001377266.1 (SEQ ID NO: 301574). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001377266.1 (SEQ ID NO: 301574).

In various embodiments, the MAPT gene product is a MAPT mRNA sequence comprising NCBI Reference Sequence NM_001377267.1 (SEQ ID NO: 301575). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001377267.1 (SEQ ID NO: 301575).

In various embodiments, the MAPT gene product is a MAPT mRNA sequence comprising NCBI Reference Sequence NM_001377268.1 (SEQ ID NO: 301576). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_001377268.1 (SEQ ID NO: 301576).

In various embodiments, the MAPT gene product is a MAPT mRNA sequence comprising NCBI Reference Sequence NM_005910.6 (SEQ ID NO: 301577). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_005910.6 (SEQ ID NO: 301577).

In various embodiments, the MAPT gene product is a MAPT mRNA sequence comprising NCBI Reference Sequence NM_016834.5 (SEQ ID NO: 301578). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_016834.5 (SEQ ID NO: 301578).

In various embodiments, the MAPT gene product is a MAPT mRNA sequence comprising NCBI Reference Sequence NM_016835.5 (SEQ ID NO: 301579). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_016835.5 (SEQ ID NO: 301579).

In various embodiments, the MAPT gene product is a MAPT mRNA sequence comprising NCBI Reference Sequence NM_016841.5 (SEQ ID NO: 301580). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA sequence comprising NCBI Reference Sequence NM_016841.5 (SEQ ID NO: 301580).

In various embodiments, the MAPT gene product is a MAPT RNA sequence comprising NCBI Reference Sequence NR_165166.1 (SEQ ID NO: 301581). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT RNA sequence comprising NCBI Reference Sequence NR_165166.1 (SEQ ID NO: 301581).

In various embodiments, the MAPT gene product is a MAPT mRNA transcript variant comprising NCBI Reference Sequence XM_005257370.4 (SEQ ID NO: 301582). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257370.4 (SEQ ID NO: 301582).

In various embodiments, the MAPT gene product is a MAPT mRNA transcript variant comprising NCBI Reference Sequence XM_005257366.3 (SEQ ID NO: 301583). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257366.3 (SEQ ID NO: 301583).

In various embodiments, the MAPT gene product is a MAPT mRNA transcript variant comprising NCBI Reference Sequence XM_005257362.4 (SEQ ID NO: 301584). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257362.4 (SEQ ID NO: 301584).

In various embodiments, the MAPT gene product is a MAPT mRNA transcript variant comprising NCBI Reference Sequence XM_005257365.4 (SEQ ID NO: 301585). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257365.4 (SEQ ID NO: 301585).

In various embodiments, the MAPT gene product is a MAPT mRNA transcript variant comprising NCBI Reference Sequence XM_005257367.4 (SEQ ID NO: 301586). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257367.4 (SEQ ID NO: 301586).

In various embodiments, the MAPT gene product is a MAPT mRNA transcript variant comprising NCBI Reference Sequence XM_005257368.4 (SEQ ID NO: 301587). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257368.4 (SEQ ID NO: 301587).

In various embodiments, the MAPT gene product is a MAPT mRNA transcript variant comprising NCBI Reference Sequence XM_005257369.4 (SEQ ID NO: 301588). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257369.4 (SEQ ID NO: 301588).

In various embodiments, the MAPT gene product is a MAPT mRNA transcript variant comprising NCBI Reference Sequence XM_005257371.4 (SEQ ID NO: 301589). In various embodiments, the MAPT gene product comprises a sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a MAPT mRNA transcript variant comprising NCBI Reference Sequence XM_005257371.4 (SEQ ID NO: 301589).

### AONs Targeting Gene Product

### PPM1A AONs

In various embodiments, a PPM1A AON disclosed herein, such as PPM1A AONs comprising a sequence of any one of SEQ ID NOs: 1-954, or a sequence of any one SEQ ID NOs: 1-954 in which one or more nucleosides are replaced with the one or more spacers, such as a sequence of any one of SEQ ID NOs: 301692-301742 or the sequences in Tables 4A-4C, target specific portions of a PPM1A gene product, such as a PPM1A mRNA or pre-mRNA transcript (e.g., any one of SEQ ID NO: 1909-1913). In some embodiments, a PPM1A AON may be an oligonucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a portion of a PPM1A gene product or to PPM1A gene sequence (e.g., any one of SEQ ID NOs: 1909-1913). In particular embodiments, a PPM1A AON may be an oligonucleotide sequence at least 85% complementary to a portion of a PPM1A gene product or to PPM1A gene sequence (e.g., any one of SEQ ID NOs: 1909-1913). In particular embodiments, a PPM1A AON may be an oligonucleotide sequence at least 90% complementary to a portion of a PPM1A gene product or to PPM1A gene sequence (e.g., any one of SEQ ID NOs: 1909-1913). In particular embodiments, a PPM1A AON may be an oligonucleotide sequence at least 95% complementary to a portion of a PPM1A gene product or to PPM1A gene sequence (e.g., any one of SEQ ID NOs: 1909-1913). In particular embodiments, a PPM1A AON may be an oligonucleotide sequence at least 98% complementary to a portion of a PPM1A gene product or to PPM1A gene sequence (e.g., any one of SEQ ID NOs: 1909-1913). In particular embodiments, a PPM1A AON may be an oligonucleotide sequence at least 99% complementary to a portion of a PPM1A gene product or to PPM1A gene sequence (e.g., any one of SEQ ID NOs: 1909-1913). In particular embodiments, a PPM1A AON may be an oligonucleotide sequence that is 100% complementary to a portion of a PPM1A gene product or to PPM1A gene sequence (e.g., any one of SEQ ID NOs: 1909-1913).

In some embodiments described herein, a PPM1A AON targets a specific portion of a PPM1A gene product, such as a PPM1A mRNA transcript or a PPM1A pre-mRNA transcript. For example, as described herein, a PPM1A AON comprises a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific portion of a PPM1A gene product, for example, a PPM1A mRNA transcript or a PPM1A pre-mRNA transcript (e.g., any one of SEQ ID NOs: 1909-1913).

In some embodiments, a PPM1A AON comprises a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a sequence of an untranslated region (UTR) of a PPM1A mRNA sequence, for example a 5′ UTR or a 3′ UTR of a PPM1A mRNA sequence. In some embodiments, a PPM1A AON comprises a sequence that is 100% complementary to a nucleotide sequence of an untranslated region (UTR) of a PPM1A mRNA sequence, for example a 5′ UTR or a 3′ UTR of a PPM1A mRNA sequence.

### ATXN2 AONs

In various embodiments, a ATXN2 AON disclosed herein, such as ATXN2 AONs with a sequence of any one of SEQ ID NOs: 1914-149354 or a sequence of any one SEQ ID NOs: 1914-149354 in which one or more nucleosides are replaced with the one or more spacers, such as a sequence of any one of SEQ ID NOs: 301692-301742 or the sequences in Tables 4A-4C), target specific portions of a ATXN2 gene product, such as a ATXN2 mRNA transcript (e.g., any one of SEQ ID NOs: 149355-149361). In some embodiments, a ATXN2 AON may be an oligonucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a portion of an ATXN2 gene product or to ATXN2 gene sequence (e.g., any one of SEQ ID NOs: 149355-149361). In particular embodiments, an ATXN2 AON may be an oligonucleotide sequence at least 85% complementary to a portion of an ATXN2 gene product or to ATXN2 gene sequence (e.g., any one of SEQ ID NOs: 149355-149361). In particular embodiments, an ATXN2 AON may be an oligonucleotide sequence at least 90% complementary to a portion of an ATXN2 gene product or to ATXN2 gene sequence (e.g., any one of SEQ ID NOs: 149355-149361). In particular embodiments, an ATXN2 AON may be an oligonucleotide sequence at least 95% complementary to a portion of an ATXN2 gene product or to ATXN2 gene sequence (e.g., any one of SEQ ID NOs: 149355-149361). In particular embodiments, an ATXN2 AON may be an oligonucleotide sequence at least 98% complementary to a portion of an ATXN2 gene product or to ATXN2 gene sequence (e.g., any one of SEQ ID NOs: 149355-149361). In particular embodiments, an ATXN2 AON may be an oligonucleotide sequence at least 99% complementary to a portion of an ATXN2 gene product or to ATXN2 gene sequence (e.g., any one of SEQ ID NOs: 149355-149361). In particular embodiments, an ATXN2 AON may be an oligonucleotide sequence that is 100% complementary to a portion of an ATXN2 gene product or to ATXN2 gene sequence (e.g., any one of SEQ ID NOs: 149355-149361).

In some embodiments described herein, an ATXN2 AON targets a specific portion of an ATXN2 gene product, such as an ATXN2 mRNA transcript or an ATXN2 pre-mRNA transcript. For example, as described herein, an ATXN2 AON comprises a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific portion of an ATXN2 gene product, for example, an ATXN2 mRNA transcript or an ATXN2 pre-mRNA transcript (e.g., any one of SEQ ID NOs: 149355-149361).

### SOD1 AONs

In various embodiments, a SOD1 AON disclosed herein, such as SOD1 AONs with a sequence of any one of 149362-158581, or SOD1 AONs with one or more spacers (e.g., a SOD1 AON with a sequence of any one SEQ ID NOs: 149362-158581 in which one or more nucleosides are replaced with the one or more spacers), such as a sequence of any one of SEQ ID NOs: 301610-301741), target specific portions of a SOD1 gene product, such as a SOD1 mRNA transcript (e.g., any one of SEQ ID NOs: 167802-167804). In some embodiments, a SOD1 AON may be an oligonucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a portion of a SOD1 gene product or to SOD1 gene sequence (e.g., any one of SEQ ID NOs: 167802-167804). In particular embodiments, a SOD1 AON may be an oligonucleotide sequence at least 85% complementary to a portion of a SOD1 gene product or to SOD1 gene sequence (e.g., any one of SEQ ID NOs: 167802-167804). In particular embodiments, a SOD1 AON may be an oligonucleotide sequence at least 90% complementary to a portion of a SOD1 gene product or to SOD1 gene sequence (e.g., any one of SEQ ID NOs: 167802-167804). In particular embodiments, a SOD1 AON may be an oligonucleotide sequence at least 95% complementary to a portion of a SOD1 gene product or to SOD1 gene sequence (e.g., any one of SEQ ID NOs: 167802-167804). In particular embodiments, a SOD1 AON may be an oligonucleotide sequence at least 98% complementary to a portion of a SOD1 gene product or to SOD1 gene sequence (e.g., any one of SEQ ID NOs: 167802-167804). In particular embodiments, a SOD1 AON may be an oligonucleotide sequence at least 99% complementary to a portion of a SOD1 gene product or to SOD1 gene sequence (e.g., any one of SEQ ID NOs: 167802-167804). In particular embodiments, a SOD1 AON may be an oligonucleotide sequence that is 100% complementary to a portion of a SOD1 gene product or to SOD1 gene sequence (e.g., any one of SEQ ID NOs: 167802-167804).

In some embodiments described herein, a SOD1 AON targets a specific portion of a SOD1 gene product, such as a SOD1 mRNA transcript or a SOD1 pre-mRNA transcript. For example, as described herein, a SOD1 AON comprises a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific portion of a SOD1 gene product, for example, a SOD1 mRNA transcript or a SOD1 pre-mRNA transcript (e.g., any one of SEQ ID NOs: 167802-167804).

### MAPT AONs

In various embodiments, a MAPT AON disclosed herein, such as MAPT AONs with a sequence of any one of SEQ ID NOs: 167805-301566, or MAPT AONs with one or more spacers (e.g., a MAPT AON with a sequence of any one SEQ ID NOs: 167805-301566 in which one or more nucleosides are replaced with the one or more spacers), such as a sequence of any one of SEQ ID NOs: 301692-301742 or the sequences in Tables 4A-4C), target specific portions of a MAPT gene product, such as a MAPT mRNA or pre-mRNA transcript (e.g., any one of SEQ ID NOs: 301567-301589). In some embodiments, a MAPT AON may be an oligonucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a portion of a MAPT gene product or to MAPT gene sequence (e.g., any one of SEQ ID NOs: 301567-301589). In particular embodiments, a MAPT AON may be an oligonucleotide sequence at least 85% complementary to a portion of a MAPT gene product or to MAPT gene sequence (e.g., any one of SEQ ID NOs: 301567-301589). In particular embodiments, a MAPT AON may be an oligonucleotide sequence at least 90% complementary to a portion of a MAPT gene product or to MAPT gene sequence (e.g., any one of SEQ ID NOs: 301567-301589). In particular embodiments, a MAPT AON may be an oligonucleotide sequence at least 95% complementary to a portion of a MAPT gene product or to MAPT gene sequence (e.g., any one of SEQ ID NOs: 301567-301589). In particular embodiments, a MAPT AON may be an oligonucleotide sequence at least 98% complementary to a portion of a MAPT gene product or to MAPT gene sequence (e.g., any one of SEQ ID NOs: 301567-301589). In particular embodiments, a MAPT AON may be an oligonucleotide sequence at least 99% complementary to a portion of a MAPT gene product or to MAPT gene sequence (e.g., any one of S SEQ ID NOs: 301567-301589). In particular embodiments, a MAPT AON may be an oligonucleotide sequence that is 100% complementary to a portion of a MAPT gene product or to MAPT gene sequence (e.g., any one of SEQ ID NOs: 301567-301589).

In some embodiments described herein, a MAPT AON targets a specific portion of a MAPT gene product, such as a MAPT mRNA transcript or a MAPT pre-mRNA transcript. For example, as described herein, a MAPT AON comprises a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific portion of a MAPT gene product, for example, a MAPT mRNA transcript or a MAPT pre-mRNA transcript (e.g., any one of SEQ ID NOs: 301567-301589).

### Nuclease-Mediated Inhibition

In one aspect, the present disclosure provides a nuclease to reduce expression of target gene products (e.g., gene products of any one of PPM1A, ATXN2, SOD1, or MAPT). In some embodiments, the nuclease can be a RNAse (e.g., RNAseH), Zinc Finger nuclease (ZFN), a meganuclease, a transcription activator-like effector nuclease (TALEN), or a clustered regularly interspaced short palindromic repeats (CRISPR) associated protein.

In certain embodiments, target gene products are inhibited, eliminated, or reduced in quantity using zinc finger nucleases (ZFNs). Synthetic ZFNs are composed of a zinc finger binding domain fused with, e.g., a FokI DNA cleavage domain. ZFNs can be designed/engineered for editing the genome of a cell, including, but not limited to, knock-out or knock-in gene expression, in a wide range of organisms. A meganuclease, a TALEN, or a CRISPR associated protein can be used for genome engineering in cells of a patient suffering from or at risk of a neurological disease, including neurons, for example, motor neurons, and other cells of the nervous system. The described reagents can be used to target promoters, protein-encoding regions (exons), introns, 5′ and 3′ UTRs, and more.

CRISPR genome editing typically comprises two distinct components: (1) a guide RNA and (2) an endonuclease, specifically a CRISPR associated (Cas) nuclease (e.g., Cas9). The guide RNA is a combination of the endogenous bacterial crRNA and tracrRNA into a single chimeric guide RNA (gRNA) transcript. Without being bound by theory, it is believed that when gRNA and the Cas are expressed in the cell, the genomic target sequence can be modified or permanently disrupted.

A gRNA/Cas complex can be recruited to a target sequence, for example, the target gene (e.g., any one of PPM1A, ATXN2, SOD1, or MAPT) by base-pairing between the gRNA sequence and the complement to the target DNA sequence in the target gene. An appropriate genomic target sequence contains a Protospacer Adjacent Motif (PAM) sequence immediately following the target sequence. The binding of the gRNA/Cas complex localizes the Cas to the target sequence, allowing wild-type Cas to cut both strands of DNA, causing a double strand break. The double strand break is repaired through one of two general repair pathways: (1) the non-homologous end joining DNA repair pathway or (2) the homology directed repair pathway. The non-homologous repair pathway can result in insertions/deletions at the double strand break that can lead to frameshifts and/or premature stop codons, effectively disrupting the open reading frame of the target gene. The homology directed repair pathway requires the presence of a repair template, which is used to fix the double strand break.

In certain embodiments, target gene expression is reduced using CRISPR genome editing. In some embodiments, a gRNA pair is used to target a target gene to reduce and/or eliminate expression of the target gene. In certain embodiments, one gRNA pair is used to reduce expression of the target gene. In certain other embodiments, multiple gRNA pairs are used to reduce expression of the target gene. gRNA pairs can be designed using known techniques and based on the gene sequence. In certain embodiments, gRNA sequences may include modifications such as 2′ O-methyl analogs and 3′ phosphorothioate internucleotide linkages in the terminal three nucleotides on both 5′ and 3′ ends of the gRNA.

### Neurological Diseases

Methods described herein may be used to treat neurological diseases including, but not limited to, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

Motor neuron diseases are a group of diseases characterized by loss of function of motor neurons that coordinate voluntary movement of muscles by the brain. Motor neuron diseases may affect upper and/or lower motor neurons, and may have sporadic or familial origins. Motor neuron diseases include amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and ALS with frontotemporal dementia.

Symptoms of motor neuron diseases include muscle decay or weakening, muscle pain, spasms, slurred speech, difficulty swallowing, loss of muscle control, joint pain, stiff limbs, difficulty breathing, drooling, and complete loss of muscle control, including over basic functions such as breathing, swallowing, eating, speaking, and limb movement. These symptoms are also sometimes accompanied by depression, loss of memory, difficulty with planning, language deficits, altered behavior, and difficulty assessing spatial relationships and/or changes in personality.

Motor neuron diseases can be assessed and diagnosed by a clinician of skill, for example, a neurologist, using various tools and tests. For example, the presence or risk of developing a motor neuron disease can be assessed or diagnosed using blood and urine tests (for example, tests that assay for the presence of creatinine kinase), magnetic resonance imaging (MRI), electromyography (EMG), nerve conduction study (NCS), spinal tap, lumbar puncture, and/or muscle biopsy. Motor neuron diseases can be diagnosed with the aid of a physical exam and/or a neurological exam to assess motor and sensory skills, nerve function, hearing and speech, vision, coordination and balance, mental status, and changes in mood or behavior.

A patient suffering from ALS, FTD, ALS with FTD, or another neurological or motor neuron disease can be a patient that is diagnosed with the disease or that displays symptoms of the disease. A patient suffering from ALS, FTD, ALS with FTD, or another neurological or motor neuron disease can be a patient that previously suffered from the disease and, after recovering or experiencing complete or partial amelioration of the disease and/or disease symptoms, experiences a complete or partial relapse of the disease or disease symptoms. A patient suffering from ALS, FTD, ALS with FTD, or another neurological or motor neuron disease or condition can be a patient that harbors a genetic mutation associated with manifestation of the disease or condition. For example, a patient suffering from ALS can be a patient that harbors a genetic mutation in any of SOD1, C9orf72, Ataxin 2 (ATXN2), Charged Multivesicular Body Protein 2B (CHMP2B), Dynactin 1 (DCTN1), Human Epidermal Growth Factor Receptor 4 (ERBB4), FIG. 4 phosphoinositide 5-phosphatase (FIG. 4), NIMA related kinase 1 (NEK1), Heterogeneous nuclear ribonucleoprotein A1 (HNRNPA1), Neurofilament Heavy (NEFH), Peripherin (PRPH), TAR DNA binding protein 43 (TDP43 or TARDP), Fused in Sarcoma (FUS), Ubiquilin-2 (UBQLN2), Kinesin Family Member 5A (KIF5A), Valosin-Containing Protein (VCP), Alsin (ALS2), Senataxin (SETX), Sigma Non-Opioid Intracellular Receptor 1 (SIGMAR1), Survival of Motor Neuron 1 (SMN1), Spastic Paraplegia 11 (SPG11), Autosomal Recessive, Transient Receptor Potential Cation Channel Subfamily M Member 7 (TRPM7), Vesicle-Associated Membrane Protein-Associated Protein B/C (VAPB), Angiogenin (ANG), Profilin-1 (PFN1), Matrin-3 (MATR3), Coiled-coil-helix-coiled-coil-helix domain Containing 10 (CHCHD10), Tubulin, Alpha 4A (TUBA4A), TBK1, C21orf2, Sequestosome-1 (SQSTM1, also known as Ubiquitin-binding protein p62), and/or optineurin (OPTN), in particular, where the mutation is associated with ALS or a high risk of developing ALS.

A patient at risk of ALS, FTD, ALS with FTD, or another neurological or motor neuron disease can include those patients with a familial history of the disease or a genetic predisposition to the disease (e.g., a patient that harbors a genetic mutation associated with high disease risk, for example), or patients exposed to environmental factors that increase disease risk. For example, a patient may be at risk of ALS if the patient harbors a mutation in any of SOD1, C9orf72, ATXN2, CHMP2B, DCTN1, ERBB4, FIG4, HNRNPA1, NEFH, PRPH, NEK1, TDP43, FUS, UBQLN2, KIF5A, VCP, ALS2, SETX, SIGMAR1, SMN1, SPG11, TRPM7, VAPB, ANG, PFN1, MATR3, CHCHD10, TUBA4A, TBK1, SQSTM1, C21orf2, and/or OPTN, in particular, where the mutation is associated with ALS or high risk of developing ALS. A patient at risk may also include those patients diagnosed with a disease or condition that has a high comorbidity with ALS, FTD, ALS with FTD, or another neurological or motor neuron disease (for example, a patient suffering from dementia, which is significantly associated with higher odds of a family history of ALS, FTD, and of bulbar onset ALS (see Trojsi, F., et al. (2017) “Comorbidity of dementia with amyotrophic lateral sclerosis (ALS): insights from a large multicenter Italian cohort” J Neurol 264: 2224-31)).

### Amyotrophic Lateral Sclerosis

ALS is a progressive motor neuron disease that disrupts signals to all voluntary muscles. ALS results in atrophy of both upper and lower motor neurons. Symptoms of ALS include weakening and wasting of the bulbar muscles, general and bilateral loss of strength, spasticity, muscle spasms, muscle cramps, fasciculations, slurred speech, and difficulty breathing or loss of ability to breathe. Some individuals with ALS also suffer from cognitive decline. At the molecular level, ALS is characterized by protein and RNA aggregates in the cytoplasm of motor neurons, including aggregates of the RNA-binding protein TDP43.

ALS is most common in males above 40 years of age, although it can also occur in women and children. Risk of ALS is also heightened in individuals who smoke, are exposed to chemicals such as lead, or who have served in the military. Most instances of ALS are sporadic, while only about 10% of cases are familial. Causes of ALS include sporadic or inherited genetic mutations, high levels of glutamate, protein mishandling. Genetic mutations associated with ALS include mutations in the genes SOD1, C9orf72, TARDP, FUS, ANG, ATXN2, CHCHD10, CHMP2B, DCTN1, ERBB4, FIG4, HNRPA1, MATR3, NEFH, OPTN, PFN1, PRPH, SETX, SIGMAR1, SMN1, SPG11, SQSTM1, TBK1, TRPM7, TUBA4A, UBQLN2, VAPB, and VCP.

### Frontotemporal Dementia

Frontotemporal dementia (FTD) is a form of dementia that affects the frontal and temporal lobes of the brain. It has an earlier average age of onset than Alzheimer's disease—40 years of age. Symptoms of FTD include extreme changes in behavior and personality, speech and language problems, and movement-related symptoms such as tremor, rigidity, muscle spasm, weakness, and difficulty swallowing. Subtypes of FTD include behavior variant frontotemporal dementia (bvFTD), characterized by changes in personality and behavior and primary progressive aphasia (PPA), which affects language skills, speaking, writing and comprehension. FTD is associated with tau protein accumulation (Pick bodies) and function of altered TDP43 function. About 30% of cases of FTD are familial, and no other risk factors other than family history of the disease are known. Genetic mutations associated with FTD include mutations in the genes C9orf72, Progranulin (GRN), microtubule-associated protein tau (MAPT), UBQLN2, VPC, CHMP2B, TARDP, FUS, ITM2B, CHCHD10, SQSTM1, PSEN1, PSEN2, CTSF, CYP27A1, TBK1 and TBP.

Amyotrophic lateral sclerosis with frontotemporal dementia (ALS with FTD) is a clinical syndrome in which FTD and ALS occur in the same individual. Interestingly, mutations in C9orf72 are the most common cause of familial forms of ALS and FTD. Additionally, mutations in TBK1, VCP, SQSTM1, UBQLN2 and CHMP2B are also associated with ALS with FTD. Symptoms of ALS with FTD include dramatic changes in personality, as well as muscle weakness, muscle atrophy, fasciculations, spasticity, dysarthria, dysphagia, and degeneration of the spinal cord, motor neurons, and frontal and temporal lobes of the brain. At the molecular level, ALS with FTD is characterized by the accumulation of TDP-43 and/or FUS proteins. TBK1 mutations are associated with ALS, FTD, and ALS with FTD.

### TBK1 and RIPK1 Function

In one aspect, methods described herein include exposing a cell to a PPM1A AON to modify the activity, function, or other characteristics of a gene or a gene product, for example, an mRNA or protein. For example, methods described herein include a method of increasing or decreasing or inhibiting the activity, function, or other characteristics of a gene or a gene product. For example, described herein is a method of increasing phosphorylation of a residue of TANK-binding kinase 1 (also known as Serine/threonine-protein kinase TBK1; “TBK1”). For example, described herein is a method of increasing TBK1 serine residue 172 (ser172) phosphorylation in a cell, where the method includes exposing the cell to a PPM1A AON. In some embodiments, TBK1 ser172 phosphorylation is increased in a cell of a patient suffering from ALS, FTD, or ALS with FTD. In some embodiments, the method of increasing TBK1 ser172 phosphorylation includes exposing a cell to a PPM1A antisense oligonucleotide of any one of SEQ ID NOs: 1-954, or a sequence of any one SEQ ID NOs: 1-954 in which one or more nucleosides are replaced with the one or more spacers, such as any one of SEQ ID NOs: 301692-301742 or the sequences in Tables 4A-4C.

Also described herein is a method of increasing TBK1 function in a cell, where the method includes exposing the cell to a PPM1A AON. For example, described herein is a method of increasing TBK1 function in a cell, where the method includes exposing the cell to a PPM1A AON. In some embodiments, TBK1 function is increased in a cell of a patient suffering from ALS, FTD, or ALS with FTD. In some embodiments, the method of increasing TBK1 function includes exposing a cell to a PPM1A antisense oligonucleotide of any one of SEQ ID NOs: 1-954, or a sequence of any one SEQ ID NOs: 1-954 in which one or more nucleosides are replaced with the one or more spacers, such as any one of SEQ ID NOs: 301692-301742 or the sequences in Tables 4A-4C.

Tank-binding kinase 1 (TBK1) is an IKK family of kinases that induces type-1 interferon activity and plays a major role in the phosphorylation of autophagy adaptors. Mutations in TBK1 are thought to result in impaired autophagy and contribute to the accumulation of protein aggregates and ALS pathology. At least 92 mutations in TBK1 have been identified in patients with ALS, FTD, or ALS with FTD (see Oakes et al., (2017) “TBK1: a new player in ALS linking autophagy and neuroinflammation” Molecular Brain 10:5, pg. 1-10). Furthermore, along with mutations in C9orf72, OPTN, SQSTM1/p62, UBQLN2, and TDP43, mutations in TBK1 account for approximately 15% of ALS and FTD patients. Furthermore, TBK1 haploinsufficiency associated with loss of function mutations has been identified as a major driver of familial ALS (see Freischmidt et al., (2015) “Haploinsufficiency of TBK1 causes familial ALS and fronto-temporal dementia” Nature Neuroscience, 18(5):631-6).

Autophagy is a process by which proteins and damaged organelles are degraded and recycled. Abnormal protein aggregates are a hallmark of ALS pathology, and mutations in several genes involved in regulating autophagy are associated with ALS (for example, SQSTM1, SOD1, OPTN, VCP, UBQLN2, and TBK1). Thus, disruption of autophagy appears to contribute to ALS pathology.

Phosphorylation of residue Ser172 of TBK1 results in conformational changes in TBK1, that allow substrate binding by the protein's kinase domain. TBK1 phosphorylates a number of autophagy adaptors, and several TBK1 mutations identified in ALS patients inhibit the ability of TBK1 to phosphorylate these adaptors. Other TBK1 mutations result in decreased mRNA and protein levels. Additionally, individuals carrying mutations in TBK1 also display TDP43-positive aggregates in various brain regions. Thus, TBK1 mutations may result in decreased autophagy and accumulation of protein aggregates in motor neurons.

PPM1A is a member of the PP2C family of Ser/Thr protein phosphatases. PP2C family members are negative regulators of cellular stress-response pathways and are involved in regulating the cell-cycle and NF-κB pathways. PPM1A also dephosphorylates and inactivates TBK1. In particular, PPM1A dephosphorylates Ser172 of TBK1. Activated TBK1 can phosphorylate RIPK1 in such a manner that RIPK1 is deactivated. Thus PPM1A activity indirectly activates RIPK1, and therefore, reducing PPM1A expression indirectly inactivates RIPK1.

The present disclosure is based in part on the finding that increasing TBK1 activity, for example, increasing TBK1 activity in an individual or the cell of an individual who is suffering from TBK1 haploinsufficiency, can be used as a mechanism to treat neurological diseases, for example, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

The disclosure is also based in part on the finding that increasing TBK1 activity, for example, increasing residual TBK1 activity in an individual and/or a cell of an individual suffering from TBK1 haploinsufficiency, can be achieved by increasing the amount of phosphorylated TBK1, for example, by increasing the amount of phosphorylated Ser172 TBK1, for example, an individual and/or a cell of an individual suffering from TBK1 haploinsufficiency. The disclosure is also based in part on the finding that increasing TBK1 activity, for example, increasing residual TBK1 activity in an individual and/or a cell of an individual suffering from TBK1 haploinsufficiency, can be achieved by increasing the ratio of phosphorylated TBK1 to total TBK1, for example, increasing the ratio of phosphorylated Ser172 TBK1 to unphosphorylated Ser172 TBK1, for example, in an individual and/or a cell of an individual suffering from TBK1 haploinsufficiency.

The disclosure is further based in part on the finding that increasing TBK1 activity (for example, increasing residual TBK1 activity in an individual and/or a cell of an individual suffering from TBK1 haploinsufficiency), increasing the amount of phosphorylated TBK1 (for example, increasing the amount of phosphorylated Ser172 TBK1, for example, in an individual and/or a cell of an individual suffering from TBK1 haploinsufficiency), and/or increasing the ratio of phosphorylated TBK1 to unphosphorylated TBK1 (for example, increasing the ratio of phosphorylated Ser172 TBK1 to unphosphorylated Ser172 TBK1, for example, in an individual and/or a cell of an individual suffering from TBK1 haploinsufficiency) can be achieved by inhibiting PPM1A activity and/or decreasing PPM1A protein levels, for example, in an individual and/or a cell of an individual suffering from a TBK1 haploinsufficiency. Without being bound by theory, it is believed that inhibiting PPM1A activity and/or decreasing PPM1A protein levels can be achieved by administering to a patient or a cell of a patient, a PPM1A AON, for example, a PPM1A AON described herein. In particular embodiments, the disclosure provides methods of inhibiting PPM1A activity and/or decreasing PPM1A protein amounts by administering to a patient or a cell of a patient (for example, a patient suffering from a neurological disease or a cell of a patient suffering from a neurological disease, for example, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism) a PPM1A AON, for example, a PPM1A AON comprising the nucleotide sequence of any one of SEQ ID NOs: 1-954, or a sequence of any one SEQ ID NOs: 1-954 in which one or more nucleosides are replaced with the one or more spacers, such as a sequence of any one of SEQ ID NOs: 301692-301742 or the sequences in Tables 4A-4C.

Additionally disclosed herein is a method of modulating activity of Receptor Interacting Serine/Threonine Kinase 1 (also known as “RIPK1”). For example, described herein is a method of modulating RIPK1 activity in a cell, where the method includes exposing the cell to a PPM1A AON. In various embodiments, modulating activity of RIPK1 can be useful for treating various diseases, including acute neuronal injury, multiple sclerosis, ALS, Alzheimer's Disease, Lysosomal Storage Diseases, Parkinson's Disease, and other human central nervous system diseases. In some embodiments, RIPK1 activity is modulated in a cell of a patient suffering from ALS, FTD, or ALS with FTD. In some embodiments, the method of modulating RIPK1 activity includes exposing a cell to a PPM1A antisense oligonucleotide of any one of SEQ ID NOs: 1-954, or a sequence of any one SEQ ID NOs: 1-954 in which one or more nucleosides are replaced with the one or more spacers, such as a sequence of any one of SEQ ID NOs: 301692-301742 or the sequences in Tables 4A-4C.

TBK1 regulates RIPK1 through direct phosphorylation on multiple sites including Thr189 to suppress RIPK1 kinase activity by blocking the interaction with its substrates. Degterev, A. et al, Targeting RIPK1 for the Treatment of Human Diseases, PNAS (2019), 116(20) 9714-9722 Therefore, increasing TBK1 function by increasing phosphorylation of a residue of TANK-binding kinase 1 can result in suppression of RIPK1 activity.

### Methods of Treatment

The disclosure contemplates, in part, treating neurological diseases (for example, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism) in a patient in need thereof comprising administering a disclosed AON. In some embodiments, provided herein are methods for treatment of a neurological disease in a patient in need thereof, comprising administering a disclosed AON. In some embodiments of the disclosure, an effective amount of a disclosed AON may be administered to a patient in need thereof to treat a neurological disease, for example, to restore autophagy in cells of a patient suffering from a neurological disease, and/or to reduce or inhibit a target gene product. In some embodiments of the disclosure, an effective amount of a disclosed AON may be administered to a patient in need thereof to increase TBK1 phosphorylation (for example TBK1 ser172 phosphorylation) in a cell and/or to increase TBK1 function (for example, TBK1 kinase function) in a cell.

In some embodiments, methods of treating a neurological disease associated with impaired autophagy and/or protein aggregation (for example, TDP-43 protein aggregation, for example, in motor neurons) in a patient in need thereof are provided comprising administering a disclosed compound. In some embodiments, treating a neurological disease comprises at least ameliorating or reducing one symptom associated with the neurological disease (for example, reducing muscle weakness in a patient with ALS). Methods of treating a neurological disease (for example, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism) in a patient suffering therefrom are provided, that include administering a disclosed AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed AON. The methods include for example, treating a subject at risk of developing a neurological disease; e.g., administering to the subject an effective amount of a disclosed AON. Neurological diseases that can be treated in this manner include amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

Methods of preventing or treating neurological diseases (for example, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism) form part of this disclosure. Such methods may comprise administering to a patient in need thereof or a patient at risk, a pharmaceutical preparation comprising an AON disclosed herein. For example, a method of preventing or treating a neurological disease is provided comprising administering to a patient in need thereof an AON disclosed herein.

Patients treated using an above method may experience a reduction of at least about 5%, 10%, 20%, 30%, 40%, 50%, 60%, 70%, 80%, 90%, or even 95% in the amount of a target gene product in a target cell (for example, a motor neuron) after administering an AON disclosed herein, after e.g. 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. Administering such an AON may be on, e.g., at least a daily basis. The AON may be administered orally. In some embodiments, the AON is administered intrathecally or intracisternally. For example, in an embodiment described herein, an AON is administered intrathecally or intracisternally about every 3 months. The delay or worsening of clinical manifestation of a neurological disease in a patient as a consequence of administering an AON disclosed here may be at least e.g., 6 months, 1 year, 18 months or even 2 years or more as compared to a patient who is not administered an AON such as one disclosed herein.

In another aspect, the disclosure provides methods of preventing, ameliorating, and/or treating a neurological disease, for example, a motor neuron disease. For example described herein are methods of preventing, ameliorating, and/or treating ALS, FTD, and ALS with FTD. In some embodiments, the disclosure provides a method of treating a neurological disease in a patient, for example, a patient in need of treatment of a neurological disease, where the method comprises administering to the patient an AON. In some embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

In some embodiments, the patient is a mammal, for example, a human, a primate, a dog, a cat, a horse, a cow, a goat, a sheep, a mouse, or a rat. In particular embodiments, the patient is a human patient, for example, a human patient in need of treatment of a neurological disease, for example, ALS, FTD, or ALS with FTD. In some embodiments, the patient is a patient at risk of developing a neurological disease, for example, ALS, FTD, or ALS with FTD. In some embodiments, the patient is a patient suffering from a neurological disease, for example, ALS, FTD, or ALS with FTD. In some embodiments, the patient is a patient exhibiting symptoms associated with a neurological disease, for example, ALS, FTD, or ALS with FTD.

In another aspect, described herein are methods of modifying or restoring cellular function or activity, for example, cellular function or activity of a motor neuron. For example, described herein is a method of modifying or restoring cellular function or activity of a motor neuron of a patient at risk of or suffering from a neurological disease, for example, amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism. In some embodiments, the method includes exposing a cell to an AON. In some embodiments, the method includes exposing the cell to an AON in vivo or ex vivo.

In an embodiment described herein, the disclosure provides a method of increasing or restoring autophagy in a cell, where the method includes exposing the cell to an AON or contacting the cell with an AON. In some embodiments, the cell is a cell of a patient in need of treatment of a neurological disease. In some embodiments, the neurological disease is any one of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism. In some embodiments, the exposing or contacting is performed in vivo or ex vivo. For example, in an embodiment described herein, a cell of a patient suffering from ALS, FTD, or ALS with FTD is exposed to or contacted with an AON, for example, an antisense oligonucleotide comprising any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C.

The AONs of the invention can be used alone or in combination with each other where by at least two AONs of the invention are used together in a single composition or as part of a treatment regimen. The AONs of the invention may also be used in combination with other drugs for treating neurological diseases or conditions.

In various embodiments, methods of treating a neurological disease comprises selecting a patient for treatment using an AON disclosed herein. Selecting a patient for treatment can include measuring the presence or level of expression of certain markers of neurological disease. Examples of markers include neurofilament light (NEFL), neurofilament heavy (NEFH), phosphorylated neurofilament heavy chain (pNFH), TDP-43, or p75^{ECD}. Such markers can be measured from the plasma, the spinal cord fluid, the cerebrospinal fluid, the extracellular vesicles (for example, CSF exosomes), the blood, the urine, the lymphatic fluid, fecal matter, or a tissue of the patient.

In particular embodiments, the patient for treatment is selected by measuring phosphorylated neurofilament heavy chain (pNFH) in cerebrospinal fluid (CSF). In particular embodiments, the pNFH in the CSF of the patient is used to predict disease status and survival in C90RF72-associated amyotrophic lateral sclerosis (c9ALS) patients after initial administration and/or during on-going treatment.

In some embodiments, selecting a patient for treatment can include determining whether the patient expresses a mutation of a disease-associated gene. For example, a disease-associated gene can be an ALS-associated gene selected from any of TBK1, TARDBP, SQSTM1, VCP, C9orf72, FUS, and CHCHD10. For example, the patient can be identified as a candidate patient for treatment according to the determination that the patient includes one or more mutations in the disease-associated genes.

In various embodiments, a patient selected for treatment can be administered an AON disclosed herein and/or or a pharmaceutical composition thereof.

### Treatment and Evaluation

In another aspect, the methods described herein include exposing a cell to an AON to inhibit or decrease activity or function of a gene or gene product, for example, an mRNA or protein. For example, described herein is a method of inhibiting expression, activity, and/or function of any of PPM1A, ATXN2, SOD1, or MAPT in a cell. In some embodiments, expression, activity, and/or function of a target is inhibited in a cell of a patient suffering from ALS, FTD, or ALS with FTD. In some embodiments, the method of inhibiting a target includes exposing a cell to an AON of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C.

In methods described herein, exposing a cell to an AON can include administering the AON, or a pharmaceutical composition that includes the AON, to a patient, for example, a patient suffering from or at risk of developing a neurological disease such as amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism. Thus, embodiments described herein can include administering an AON, or a pharmaceutical composition that includes an AON, to a patient in need of treatment, for example, a patient suffering from or at risk of developing a neurological disease such as amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism. Methods described herein embrace methods of administering an AON that allow administration of a therapeutically effective amount of the AON to a patient, for example, to a cell of a patient and/or to a site for treatment of a patient. For example, methods described herein include, but are not limited to, methods where an AON, or a pharmaceutical composition that includes an AON, is administered topically, parenterally, orally, buccally, sublingually, pulmonarily, intrathecally, intracisternally, intratracheally, intrathalamically, intracerebroventricularly, intraocularly, intranasally, transdermally, rectally, vaginally, or intraduodenally. In particular embodiments, the AON is administered orally. In some embodiments, the AON is administered intrathecally or intracisternally. In embodiments described herein, the methods include administering a therapeutically effective amount of an AON, for example, a therapeutically effective amount of an AON.

The methods described herein include methods of administering to a patient and/or exposing a cell to an AON, for example, an AON of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, or a pharmaceutically acceptable salt thereof.

In a further aspect, described herein is a use of an AON in the manufacture of a medicament for the treatment of neurological disease. For example, described herein is a use of an AON in the manufacture of a medicament for the treatment of ALS, FTD, or ALS with FTD. In some embodiments, the AON for use in the manufacture of a medicament for treatment is an AON of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, or a pharmaceutically acceptable salt thereof.

In a further aspect, described herein is a method of treating a neurological disease in a patient in need thereof, where the method includes administering to the patient in need thereof a pharmaceutical composition comprising a therapeutically effective amount of an AON, and a pharmaceutically acceptable excipient. In some embodiments, the neurological disease is amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism. In some embodiments, the AON includes any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, or a pharmaceutically acceptable salt thereof.

In embodiments described herein, the pharmaceutical composition comprising a therapeutically effective amount of an AON, and a pharmaceutically acceptable excipient can be administered in any number of ways to achieve therapeutic delivery to a cell of a patient and/or to a site for treatment of a patient in need thereof. For example, in embodiments described herein, a pharmaceutical composition comprising a therapeutically effective amount of an AON, and a pharmaceutically acceptable excipient can be administered topically, parenterally, intrathecally, orally, pulmonarily, intratracheally, intranasally, transdermally, buccally, intrathalamically, intracerebroventricularly, intraocularly, sublingually, rectally, vaginally, or intraduodenally. In particular embodiments, the pharmaceutical composition is administered orally. In some embodiments, the pharmaceutical composition is administered intrathecally or intracisternally. In embodiments described herein, the patient is a mammal, for example, a human patient.

In some embodiments, an AON described herein is for use as a medicament. For example, described herein is AON of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, or a pharmaceutically acceptable salt thereof, for use as a medicament.

In some embodiments, an AON disclosed herein, is for use in the treatment of a neurological disease. For example, described herein is an AON, or a pharmaceutically acceptable salt thereof, for use in the treatment of a neurological disease. In some embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

A patient, as described herein, refers to any animal at risk for, suffering from or diagnosed with a neurological disease, including, but not limited to, mammals, primates, and humans. In certain embodiments, the patient may be a non-human mammal such as, for example, a cat, a dog, or a horse. A patient may be an individual diagnosed with a high risk of developing a neurological disease, someone who has been diagnosed with a neurological disease, someone who previously suffered from a neurological disease, or an individual evaluated for symptoms or indications of a neurological disease, or any of the signs or symptoms associated with neurological diseases such as amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

“A patient in need,” as used herein, refers to a patient suffering from any of the symptoms or manifestations of a neurological disease, a patient who may suffer from any of the symptoms or manifestations of a neurological disease, or any patient who might benefit from a method of the disclosure for treating a neurological disease. A patient in need may include a patient who is diagnosed with a risk of developing a neurological disease, a patient who has suffered from a neurological disease in the past, or a patient who has previously been treated for a neurological disease.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed AON is the amount of the AON necessary to treat a neurological disease in a patient such that administration of the agent prevents a neurological disease from occurring in a subject, prevents neurological disease progression (e.g., prevents the onset or increased severity of symptoms of the neurological disease such as muscle weakening, spasms, or fasciculation), or relieves or completely ameliorates all associated symptoms of a neurological disease, e.g., causes regression of the disease.

Efficacy of treatment may be evaluated by means of evaluation of gross symptoms associated with a neurological disease, analysis of tissue histology, biochemical assay, imaging methods such as, for example, magnetic resonance imaging, or other known methods. For instance, efficacy of treatment may be evaluated by analyzing gross symptoms of the disease such as changes in muscle strength and control or other aspects of gross pathology associated with a neurological disease following administration of a disclosed AON to a patient suffering from a neurological disease.

Efficacy of treatment may also be evaluated at the tissue or cellular level, for example, by means of obtaining a tissue biopsy (e.g., a brain, spinal, muscle, or motor neuron tissue biopsy) and evaluating gross tissue or cell morphology or staining properties, or by obtaining a biofluid (e.g., cerebrospinal fluid, exosomes, plasma, or urine) and examining target expression in the fluid using a biochemical assay that examines protein or RNA expression. Such biochemical assays can include ddPCR, qRT-PCR, western blot, ELISA, and/or SIMOA. For instance, one may evaluate levels of a protein (e.g., e.g., protein levels of any of PPM1A, ATXN2, SOD1, or MAPT) indicative of a disease or a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or ddPCR), qPCR etc.) reaction. One may also evaluate the presence or level of expression of useful biomarkers (e.g., neurofilament light (NEFL), neurofilament heavy (NEFH), SOD1/SOD1 transcripts, TDP-43 or p75 extracellular domain (p75^{ECD})) found in spinal cord fluid, cerebrospinal fluid, plasma, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state and efficacy of treatment. Additional measurements of efficacy may include strength duration time constant (SDTC), short interval cortical inhibition (SICI), dynamometry, accurate test of limb isometric strength (ATLIS), compound muscle action potential (CMAP), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in amyotrophic lateral sclerosis (ALS) patients. CSF pNFH can serve as a prognostic biomarker for clinical trials, which will increase the likelihood of successfully developing a treatment for ALS. In various embodiments, efficacy may be determined via any of MRI, fMRI, PET, EEG, or EMG.

In some embodiments, in evaluating the efficacy of a treatment against Alzheimer's disease, mental performance can be used for measuring efficacy such as with the Mini-Mental State Examination (MMSE). For measuring efficacy, the Functional Assessment Staging Test (FAST), the Motor Screening Task, Paired Associates Learning, Spatial Working Memory, Reaction time, Rapid Visual Information Processing, Delayed Matching to Sample, Pattern Recognition Memory can be used.

In some embodiments, in evaluating the efficacy of a treatment against Parkinson's disease, the Unified Parkinson's Disease Rating Scale (UPDRS) can be implemented as the performance measure. Other measures for quantifying aspects of functional performance not measured by the UPDRS can include the Berg Balance Scale (BBS), Forward Functional Reach Test (FFR), Backward Functional Reach Test (BFR), Timed “Up & Go” Test (TUG), and gait speed.

In evaluating efficacy of treatment, suitable controls may be chosen to ensure a valid assessment. For instance, one can compare symptoms evaluated in a patient with a neurological disease following administration of a disclosed AON to those symptoms in the same patient prior to treatment or at an earlier point in the course of treatment or in another patient not diagnosed with the neurological disease. Alternatively, one may compare the results of biochemical or histological analysis of tissue following administration of a disclosed AON with those of tissue from the same patient or from an individual not diagnosed with the neurological disease or from the same patient prior to administration of the AON. Additionally, one may compare blood, plasma, serum, cell, urine, lymphatic fluid, spinal cord fluid, cerebrospinal fluid, or fecal samples following administration of the AON with comparable samples from an individual not diagnosed with the neurological disease or from the same patient prior to administration of the AON. In some embodiments one may compare extracellular vesicles (for example CSF exosomes), following administration of the AON with extracellular vesicles from an individual not diagnosed with the neurological disease or from the same patient prior to administration of the AON.

Validation of target inhibition may be determined by direct or indirect assessment of target expression levels or activity. For instance, biochemical assays that measure any of PPM1A, ATXN2, SOD1, or MAPT protein or RNA expression may be used to evaluate overall inhibition. For instance, one may measure target protein levels in cells or tissue by Western blot to evaluate overall levels. One may also measure target mRNA levels by means of Northern blot or quantitative polymerase chain reaction to determine overall target inhibition. One may also evaluate target protein levels or levels of another protein indicative of the target signaling activity in dissociated cells, non-dissociated tissue, extracellular vesicles (for example, CSF exosomes), blood, serum, or fecal matter via immunocytochemical or immunohistochemical methods.

Methods of treatment disclosed herein include methods of increasing or restoring autophagy in a cell. “Autophagy” refers to the natural, regulated mechanism of the cell that disassembles unnecessary or dysfunctional components, allowing orderly degradation and recycling of cellular components. Autophagy is generally responsible for degrading relatively long-lived, cytoplasmic proteins, soluble and insoluble misfolded proteins, and also entire organelles. Failure in autophagy machinery is thought to contribute to the formation of toxic protein aggregates in motor neurons (See Ramesh and Pandley, (2017) “Autophagy Dysregulation in ALS: When Protein Aggregates Get Out of Hand” Front Mol Neurosci. 10 (Article 263)). Dysregulation of autophagy and protein aggregation and mislocalization is implicated in neurological diseases, including ALS. Methods of increasing or restoring autophagy include methods that reduce expression levels of targets in a patient suffering from a neurological disease. Methods of increasing or restoring autophagy also include methods that increase TBK1 activity or expression or TBK1 phosphorylation (for example, TBK1 ser172 phosphorylation) in cells of a patient suffering from a neurological disease.

The disclosure also provides methods of inhibiting target gene products (e.g., PPM1A, ATXN2, SOD1, or MAPT) in cells of a patient suffering from a neurological disease. Target gene products may be inhibited in any cell in which the target expression or activity occurs, including cells of the nervous system (including the central nervous system, the peripheral nervous system, motor neurons, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### Pharmaceutical Compositions and Routes of Administration

The present disclosure also provides methods for treating a neurological disease via administration of a pharmaceutical composition comprising a disclosed AON. In another aspect, the disclosure provides a pharmaceutical composition for use in treating a neurological disease. The pharmaceutical composition may be comprised of a disclosed antisense oligonucleotide and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat a neurological disease. In some embodiments, contemplated herein are pharmaceutical compositions comprising a disclosed AON and a pharmaceutically acceptable carrier. In another aspect, the disclosure provides use of a disclosed AON in the manufacture of a medicament for treating a neurological disease. “Medicament.” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In some embodiments, an AON of the disclosure, is in the form of a pharmaceutically acceptable salt. AONs described herein that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of an AON of any of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C.

Also described herein are pharmaceutical compositions comprising an AON and a pharmaceutically acceptable excipient. For example, a pharmaceutical composition described herein can include an AON, for example, an AON of any one of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs. 301692-301742 or the sequences in Tables 4A-4C, and a pharmaceutically acceptable excipient.

In some embodiments, an AON can be encapsulated in a nanoparticle coating. It is believed that nanoparticle encapsulation prevents AON degradation and enhances cellular uptake. For example, in some embodiments an AON is encapsulated in a coating of a cationic polymer, for example, a synthetic polymer (e.g., poly-L-lysine, polyamidoamine, a poly(O-amino ester), and polyethyleneimine) or a naturally occurring polymer (e.g., chitosan and a protamine). In some embodiments, an AON is encapsulated in a lipid or lipid-like material, for example, a cationic lipid, a cationic lipid-like material, or an ionizable lipid that is positively charged only at an acidic pH. For example, in some embodiments, an AON is encapsulated in a lipid nanoparticle that includes hydrophobic moieties, e.g., cholesterol and/or a polyethylene glycol (PEG) lipid.

In some embodiments, an AON is conjugated to a bioactive ligand. For example, in some embodiments described herein, an AON is conjugated to a peptide, a lipid, N-acetylgalactosamine (GalNAc), cholesterol, vitamin E, an antibody, or a cell-penetrating peptide (for example, transactivator of transcription (TAT) and penetratine).

Pharmaceutical compositions containing a disclosed AON, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

In one embodiment, a disclosed AON and any pharmaceutical composition thereof may be administered by one or several routes, including topically, parenterally, intrathecally, orally, pulmonarily, intratracheally, intranasally, transdermally, buccally, intrathalamically, intracerebroventricularly, intraocularly, sublingually, rectally, vaginally, or intraduodenally. The term parenteral as used herein includes subcutaneous injections, intrapancreatic administration, intravenous, intracisternal, intrathecal, intramuscular, intraperitoneal, intrasternal injection or infusion techniques. For example, a disclosed AON may be administered subcutaneously to a subject. In another example, a disclosed AON may be administered orally to a subject. In another example, a disclosed AON may be administered directly to the nervous system, or specific regions or cells of the nervous system (e.g., the brain, brain stem, lower motor neurons, spinal cord, upper motor neurons) via parenteral administration, for example, a disclosed AON may be administered intrathecally or intracisternally.

It will be appreciated that the AON administered to the patient having or at risk of a neurological disease in methods described herein, can be administered by various administration routes. In various embodiments, the AON can be administered by one or several routes, including orally (e.g., by inhalation spray), topically, vaginally, rectally, intrathecally, intratracheally, intracisternally, intranasally, transdermally, intrathalamically, intracerebroventricularly, intraocularly, intraduodenally, buccally, sublingually, parenterally, e.g., by subcutaneous injection. The term parenteral as used herein includes subcutaneous injections, intrapancreatic administration, and intravenous, intrathecal, intracisternal, intramuscular, intraperitoneal, and intrasternal injection or infusion techniques.

### Parenteral Administration

The pharmaceutical compositions of the disclosure can be formulated for parenteral administration, e.g., formulated for injection via the intravenous, intracisternal, intramuscular, subcutaneous, intrathecal, intralesional, or intraperitoneal routes. The preparation of an aqueous composition, such as an aqueous pharmaceutical composition containing a disclosed AON, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed AON may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed AON in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed AON to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

### Intrathecal Administration

In some embodiments, an AON, or a pharmaceutical composition of the disclosure that includes an AON, is delivered to the CNS through intrathecal administration, thereby ensuring delivery into the cerebrospinal fluid (CSF) of a patient in need of treatment. In various embodiments, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. In some embodiments, “intrathecal administration” or “intrathecal delivery” according to the present invention refers to IT administration or delivery via the lumbar area or region, e.g., lumbar IT administration or delivery. As used herein, the term “lumbar region” or “lumbar area” refers to the area between the third and fourth lumbar (lower back) vertebrae and, more inclusively, the L2-S1 region of the spine.

In various embodiments, compositions comprising a disclosed AON can be suitable for intrathecal delivery. For example, a composition suitable for intrathecal delivery can comprise the AON and any of cerebrospinal fluid, artificial cerebrospinal fluid, phosphate buffered saline (PBS), salt buffer, Ringer's solution, and Lactated Ringer's solution.

### Oral Administration

In some embodiments, contemplated herein are compositions suitable for oral delivery of a disclosed AON, e.g., tablets that include an enteric coating, e.g., a gastro-resistant coating, such that the compositions may deliver an AON to, e.g., the gastrointestinal tract of a patient.

For example, a tablet for oral administration is provided that comprises granules (e.g., is at least partially formed from granules) that include a disclosed AON, e.g., an AON of any of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed AON, e.g. an AON of any of SEQ ID NOs: 1-954, 1914-149354, 149362-158581, or 167805-301566 or an AON with one or more spacers that replace one or more nucleosides of a sequence of any one SEQ ID NOs: 1-954, 1914-149354, 149362-158581, or 167805-301566, such as any one of SEQ ID NOs: 301692-301742 or the sequences in Tables 4A-4C, and a pharmaceutically acceptable salt (e.g., a pharmaceutically acceptable filler). For example, a disclosed AON and a filler may be blended together, optionally, with other excipients, and formed into granules. In some embodiments, the intragranular phase may be formed using wet granulation, e.g. a liquid (e.g., water) is added to the blended AON compound and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed AON and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g. about 0.5% to about 10%, or about 1% to about 20%, by weight of a disclosed AON or a pharmaceutically acceptable salt thereof. Such a tablet may include for example, about 0.5% to about 60% by weight of mannitol, e.g. about 30% to about 50% by weight mannitol, e.g. about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed AON, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In another contemplated embodiment, a pharmaceutical tablet formulation for oral administration of a disclosed AON comprises an intra-granular phase, wherein the intra-granular phase includes a disclosed AON or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed AON, e.g., a disclosed AON or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed AON, e.g., a disclosed AON or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO 2010/054826, which is hereby incorporated by reference in its entirety).

The rate at which the coating dissolves and the active ingredient is released is its dissolution rate. In an embodiment, a contemplated tablet may have a dissolution profile, e.g. when tested in a USP/EP Type 2 apparatus (paddle) at 100 rpm and 37° C. in a phosphate buffer with a pH of 7.2, of about 50% to about 100% of the AON releasing after about 120 minutes to about 240 minutes, for example after 180 minutes. In another embodiment, a contemplated tablet may have a dissolution profile, e.g. when tested in a USP/EP Type 2 apparatus (paddle) at 100 rpm and 37° C. in diluted HCl with a pH of 1.0, where substantially none of the AON is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the AON releasing after 30 minutes.

In some embodiments, methods provided herein may further include administering at least one other agent that is directed to treatment of diseases and disorders disclosed herein. In one embodiment, contemplated other agents may be co-administered (e.g., sequentially or simultaneously).

### Dosage and Frequency of Administration

Exemplary formulations include dosage forms that include or consist essentially of about 35 mg to about 500 mg of a disclosed AON, such as any of a PPM1A AON, ATXN2 AON, SOD1 AON, or MAPT AON. For example, formulations that include about 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed AON are contemplated herein. In one embodiment, a formulation may include about 40 mg, 80 mg, or 160 mg of a disclosed AON. In some embodiments, a formulation may include at least 100 μg of a disclosed AON. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed AON.

In some embodiments, methods described herein include administering at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of an AON. In some embodiments, methods of the invention include administering from 35 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a AON.

The amount administered will depend on variables such as the type and extent of disease or indication to be treated, the overall health and size of the patient, the in vivo potency of the AON, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once per month. In some embodiments, dosing is once every 3 months.

### Combination Therapies

In various embodiments, an AON as disclosed herein can be administered in combination with one or more additional therapies. The combination therapy of the disclosed oligonucleotide and the one or more additional therapies can, in some embodiments, be synergistic in treating any of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

Example additional therapies for treating amyotrophic lateral sclerosis (ALS), frontotemporal dementia (FTD), or ALS with FTD include any of Riluzole (Rilutek), troriluzole, Edaravone (Radicava), rivastigmine, donepezil, QRL-101, QRL-201, galantamine, selective serotonin reuptake inhibitor, antipsychotic agents, cholinesterase inhibitors, memantine, benzodiazepine antianxiety drugs, AMX0035 (ELYBRIO®), ZILUCOPLAN (RA101495), dual AON intrathecal administration (e.g., BIIB067, BIIB078), BUB100, levodopa/carbidopa, dopaminergic agents (e.g., ropinirole, pramipexole, rotigotine), medroxyprogesterone, KCNQ2/KCNQ3 openers, Pridopidine, PrimeC (combination of ciprofloxacin and Celebrex), olanzapine (Zyprexa), quetiapine (Seroquel), SSRIs, divalproex sodium (Depakote), carbamazepine (Tegretol), medroxyprogestrone, lithium, anticonvulsants and psychostimulant agents. Additional therapies can further include breathing care, physical therapy, occupational therapy, speech therapy, and nutritional support. In various embodiments, an additional therapy can be a second antisense oligonucleotide. As an example, the second antisense oligonucleotide may be a second AON that targets a transcript.

A combination therapy (e.g., in combination with an AON) may be selected according to the disease that is to be treated. For example, for treating Alzheimer's Disease, any Memantine, Rivastigmine, Galantamine, Donepezil, Aricept®, Exelon® (Rivastigmine), Razadyne®, Aducanumab, BAN2401, BIIB091 (gosuranemab), BIIB076, BIIB080 (IONIS-MAPTRx), Elayta (CT1812), MK1942, allogenic hMSC, nilotinib, ABT-957, acitretin, ABT-354, GV1001, Riluzole, CAD106, CNP520, AD-35, Rilapladib, DHP1401, T-817 MA, TC-5619, TPI-287, RVT-101, LY450139, JNJ-54861911, Dapagliflozin, GSK239512, PF-04360365, ASP0777, SB-742457 (a 5-HT6 receptor antagonist), PF-03654746 (an H₃ receptor antagonist), GSK933776 (an Fc-inactivated anti-β amyloid (AD) monoclonal antibody (mAb)), Posiphen ((+)-phenserine tartrate), AMX0035 (ELYBRIO®), coenzyme Q10, aducanamab (ADUHLEM), memantine (NAMENDA), Namzeric, Suvorexant (belsomra), lecanemab, or any combination thereof can be selected as an additional therapy.

For example for treating Parkinson's Disease, any of Levodopa, Carbidopa-levidopa, pramipexole (MIRAPEX), ropinirole (REQUIP), rotigotine (NEUPRO), apomorphine (APOKYN, KYNMOBI), selegiline (EDLEPRYL, ZELAPAR), rasagiline, entacapone (COMTAN), tolcapone (TASMAR), amantadine (GOCOVRI, SYMMETREL, OSMOLEX), trihexyphenidyl (ARTANE), BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, istradefylline (NOURIANZ), safmamide (XADAGO), benztropine (COGENTIN), opicapone (ongentys), exenatide, lingzhi, Caffeine, sarizotan, embryonic dopamine cell implantation, or any combination thereof can be selected as an additional therapy.

For example, for treating progressive supranuclear palsy (PSP), any of UCB0107, ABBV-8E12, F-18 AV1451, BIIB092, C2N-8EI2, tideglusib, deep transcranial magnetic stimulation, lipoic acid, tolfenamica acid, lithium, AZP2006, Glial Clell Line-Derived Neurotrophic Factor, NBMI, suvorxant, zolpidem, TPI 287, davunetide, pimavanserin, Levodopa, Carbidopa-levidopa, pranipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine. CLR4001, TRX4204, Yohimbine, coenzyme Q10, OXB-102, duloxetine, pioglitazone, preladenant, or any combination thereof can be selected as an additional therapy.

For example, for treating epilepsy, any of Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), zonisamide (zonegran), or any combination thereof can be selected an additional therapy.

For example for treating Huntington's Disease, any of Tetrabenazine, deutetrabenazine, physical therapy, risperidone, haloperidol, chlorpromazine, clonazepam, diazepam, benzodiazepines, selective serotonin reuptake inhibitors, quetiapine, carbatrol, valproate, lamotrigine, pridopidine, delta-9-tetrahydrocannabinol, cannabidiol, stem-cell therapy, ISIS-443139, nilotinib, resveratrol, neflamapimod, fenofibrate, creatine, RO7234292, SAGE-718, WVE-120102, WVE-120101, dimebon, minocycline, deep brain stimulation, ursodiol, coenzyme Q10, OMS643762, VX15/2503, PF-02545920, BN82451B, SEN0014196, olanzapine, tiapridal (tiapride), or any combination thereof, can be selected as an additional therapy.

For example, for treating brain trauma, any of anticoagulants, antidepressants, muscle relaxants, stimulants, anticonvulsants, anti-anxiety medication, erythropoietin, hyperbaric treatment, rehabilitation therapies (e.g., physical, occupational, speech, psychological, or vocational counseling), or any combination thereof can be selected as an additional therapy.

For example, for treating spinal cord injury, any of AXER-204, glyburide, 5-hydroxytryptophan (5-HTP), L-3,4-dihydroxyphenylalanine (L-DOPA), or rehabilitation therapies (e.g., physical therapy, occupational therapy, recreational therapy, use of assistive devices, improved strategies for exercise and healthy diets), or any combination thereof can be selected as an additional therapy.

For example, for treating corticobasal degeneration, any of TPI-287, lithium, occupational, physical, and speech therapy, or any combination thereof can be selected as an additional therapy.

For example, for treating neuropathies, such as a chemotherapy induced neuropathy, any of gabapentin, pregabalin, lamotrigine, carbamazepine, duloxetine, gabapentinoids, tricyclic antidepressants, serotonin-norepinephrine reuptake inhibitors, opioids, neurotoxin, dextromethorphan, nicotinamide riboside, auto-antibodies targeting neuronal antigens (TS-HDS and FGFR3), or any combination thereof can be selected as an additional therapy.

For example, for treating spinal muscular atrophy, any of nusinersen (SPINRAZA), onasemnogene abeparvovec-xioi (ZOLGENSMA), risdiplam (EVRYSDI), or a combination thereof can be selected as an additional therapy.

For example, for treating spinocerebellar ataxia, any of amantadine, armodafinil, baclofen, buspirone, carbamazepine, citalopram, clonazepam, desvenlafaxine, diazepam, duloxetine, escitalopram, flunarizine, fluoxetine, fluvoxamine, gabapentin, isoniazid, levetiracetam, levodopa, memantine, modafinil, ondansetron, paroxetine, pramipexole, primidone, riluzole, ropinirole, sertraline, tizanidine, topiramate, trihexyphenidyl, valproic acid, venlafaxine, BHV-4157, or a combination thereof can be selected as an additional therapy.

For example, for treating Niemann-Pick disease type C, any of anti-seizure medications, speech therapy, physical therapy, occupational therapy, Adrabetadex, Arimoclomol, N-Acetyl-L-Leucine, or any combination thereof can be selected as an additional therapy.

For example, for treating Charcot-Marie-Tooth Disease (CMT), any of physical and occupational therapies, orthopedic surgery, orthopedic devices, PXT3003, or any combination thereof can be selected as an additional therapy.

For example, for treating Mucopolysaccharidosis type II (MPSIIA), any of enzyme replacement therapy: idursulfase (Elaprase), surgical intervention (tonsillectomy and/or adenoidectomy), RGX-121 gene therapy, adalimumab, MT2013-31, or any combination thereof can be selected as an additional therapy.

For example, for treating Mucolipidosis IV, any of physical, occupational, and speech therapies, contact lenses and artificial tears, genetic counseling, or any combination thereof can be selected as an additional therapy.

For example, for treating GM1 gangliosidosis, any of anticonvulsants, physical and occupational therapies, galactosidase, gene delivery of galactosidase, LYS-GM101 gene therapy, or any combination thereof can be selected as an additional therapy.

For example, for treating Sporadic inclusion body myositis (sIBM), any of physical and occupational therapies, use of devices such as braces, walkers, wheelchairs, immunosuppressants, BYM338, or any combination thereof can be selected as an additional therapy.

For example, for treating Henoch-Schonlein purpura (HSP), any of corticosteroids, colchicine, dapsone, azathioprine, or any combination thereof can be selected as an additional therapy.

For example, for treating Gaucher's disease, any of enzyme replacement therapy, substrate reduction therapy, N-acetylcysteine, GZ/SAR402671, cerezyme, or any combination thereof can be selected as an additional therapy.

In various embodiments, the disclosed oligonucleotide and the one or more additional therapies can be conjugated to one another and provided in a conjugated form. Further description regarding conjugates involving the disclosed oligonucleotide is described below. When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. In various embodiments, the disclosed oligonucleotide and one or more additional therapies are provided concurrently. In various embodiments, the disclosed oligonucleotide and one or more additional therapies are provided simultaneously. In various embodiments, the disclosed oligonucleotide and one or more additional therapies are provided sequentially.

### Conjugates

In certain embodiments, provided herein are oligomeric compounds, which comprise an oligonucleotide (e.g., AON) and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### Conjugate Groups

In certain embodiments, an AON is covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In particular embodiments, conjugate groups modify the circulation time (e.g., increase) of the oligonucleotides in the bloodstream such that increased concentrations of the oligonucleotides are delivered to the brain. In particular embodiments, conjugate groups modify the residence time (e.g., increase residence time) of the oligonucleotides in a target organ (e.g., brain) such that increased residence time of the oligonucleotides improves their performance (e.g., efficacy). In particular embodiments, conjugate groups increase the delivery of the oligonucleotide to the brain through the blood brain barrier and/or brain parenchyma (e.g., through receptor mediated transcytosis). In particular embodiments, conjugate groups enable the oligonucleotide to target a specific organ (e.g., the brain). In certain embodiments, conjugate groups impart anew property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therap, Nucleic Acids, 2015, 4, e220; and Nishina et al., Molecular Therapy, 2008, 16, 734-740), or a GalNAc cluster (e.g., WO2014/179620).

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, bile acids, phenylbutyrate, fluorophores, and dyes. In particular embodiments, conjugate moieties are selected from a peptide, a lipid, N-acetylgalactosamine (GalNAc), cholesterol, vitamin E, lipoic acid, panthothenic acid, polyethylene glycol, an antibody (e.g., an antibody for crossing the blood brain barrier such as anti-transferrin receptor antibody), or a cell-penetrating peptide (e.g., transactivator of transcription (TAT) and penetratine).

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

### Conjugate Linkers

Conjugate moieties are attached to an AON through conjugate linkers. In certain oligomeric compounds, the conjugate linker is a single chemical bond (e.g., the conjugate moiety is attached directly to an oligonucleotide through a single bond). In certain embodiments, the conjugate linker comprises a chain structure, an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 3 linker-nucleosides.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, it is desirable for a conjugate group to be cleaved from the AON. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxy adenosine.

### Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### Diagnostic Methods

The disclosure also provides a method of diagnosing a patient with a neurological disease that relies upon detecting levels of target expression signal in one or more biological samples of a patient. As used herein, the term “target expression signal” can refer to any indication of target gene expression, or gene or gene product activity (e.g., target gene expression, or gene or gene product activity for any one of PPM1A, ATXN2, SOD1, or MAPT). Gene products include RNA (e.g., mRNA), peptides, and proteins. Indices of gene expression that can be assessed include, but are not limited to, gene or chromatin state, gene interaction with cellular components that regulate gene expression, gene product expression levels (e.g., RNA expression levels, protein expression levels), or interaction of RNA or protein with transcriptional, translational, or post-translational processing machinery. Indices of gene product activity include, but are not limited to, assessment of signaling activity (e.g., assessment of protein signaling activation or phosphorylation).

Detection of target expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, extracellular vesicles (for example, CSF exosomes), or cells of a patient. Detection may be achieved by measuring target expression signal in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Biochemical assays that examine protein or RNA expression may also be used for detection. For instance, one may evaluate levels of a protein indicative of a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or ddPCR), qPCR etc.) reaction.

One may also evaluate the presence or level of expression of useful biomarkers (e.g., neurofilament light (NEFL), neurofilament heavy (NEFH), SOD1/SOD1 transcripts, TDP-43 or p75 extracellular domain (p75^{ECD})) found in spinal cord fluid, cerebrospinal fluid, plasma, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state. Additional measurements may include strength duration time constant (SDTC), short interval cortical inhibition (SICI), dynamometry, accurate test of limb isometric strength (ATLIS), compound muscle action potential (CMAP), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in amyotrophic lateral sclerosis (ALS) patients. CSF pNFH can serve as a prognostic biomarker for clinical trials, which will increase the likelihood of successfully developing a treatment for ALS.

In some embodiments, diagnosing a patient with a neurological disease such as Alzheimer's disease can involve evaluating mental performance of the patient. Evaluation of mental performance can involve a Mini-Mental State Examination (MMSE). Additional examples for measuring mental performance include the Functional Assessment Staging Test (FAST), the Motor Screening Task, Paired Associates Learning, Spatial Working Memory, Reaction time, Rapid Visual Information Processing, Delayed Matching to Sample, and Pattern Recognition Memory In some embodiments, diagnosing a patient with a neurological disease such as Parkinson's disease involves implementing the Unified Parkinson's Disease Rating Scale (UPDRS) as the performance measure. Other measures for quantifying aspects of functional performance not measured by the UPDRS can include the Berg Balance Scale (BBS), Forward Functional Reach Test (FFR), Backward Functional Reach Test (BFR), Timed “Up & Go” Test (TUG), and gait speed.

### Modifications in General

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or $ such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool.

### EXAMPLES

The disclosure is further illustrated by the following examples. The examples are provided for illustrative purposes only, and are not to be construed as limiting the scope or content of the disclosure in any way.

### Example 1: Design and Selection of PPM1A Gapmer Antisense Oligonucleotides

PPM1A antisense oligonucleotides were evaluated in iPSC derived human motor neurons (hMN) purchased from FujiFilm Cellular Dynamics (Catalog #: C1050). The cells were seeded in 96-well plates at a density of 10,000 cells/well. Antisense oligonucleotide (AON) to PPM1A were transfected with Endoporter (Gene Tools, Philomath, OR, USA) to decrease expression of PPM1A transcript. Vehicle control consisted of motor neuron treatment with Endoporter alone.

To evaluate PPM1A AON ability to knock down PPM1A mRNA transcripts, antisense oligonucleotides to PPM1A were incubated with Endoporter in media before addition to the cells. After 72 hours, cells lysate was prepared and collected from the 96-well plates for RT-qPCR. From the cell lysates, cDNA was generated, and multiplexed RT-qPCR assay performed with Taqman probes for PPM1A and reference GAPDH quantification.

Using the Applied Biosystems® PCR Thermal Cycler (SimpliAmp), cDNA was prepared directly from the cell lysates by performing one cycle of reverse transcription at 37° C. for 30 minutes, followed by heat inactivation of the RT enzyme at 95° C. for 5 minutes.

Transcript levels were detected by RT-qPCR using Taqman. Specifically, RT-qPCR was performed for detecting GAPDH using Thermofisher TaqMan Gene Expression Assay Hs_03929097_g1. RT-qPCR was performed for detecting PPM1A using Thermofisher TaqMan Gene Expression Assay Hs06637123_g1.

RT-qPCR was performed on Applied Biosystems® QuantStudio 6 Flex Real-Time PCR System. One cycle of uracil-N-glycosylase (UNG) incubation was performed at a temperature of 50° C. for 2 min. One cycle of polymerase activation/initial denaturation was performed at a temperature of 95° C. for 20 seconds. Forty cycles of amplification were performed at a temperature of 95° C. for 1 second followed by 60° C. for 20 seconds.

PPM1A signal cycle threshold (Ct) was normalized to GAPDH (ΔCt). To visualize the quantitative changes (e.g., ratio of PPM1A transcript decrease), the normalized PPM1A signal was further normalized to the mean vehicle signal (treated with Endoporter alone, ΔΔCt). Relative quantity (RQ) of transcript level was calculated using the equation RQ=2^{-ΔΔCt} and is used to describe the treatment condition comparison to normal, healthy levels.

PPM1A AON knock-down of PPM1A transcripts were presented as mean relative quantity (RQₘₑₐₙ) of the technical triplicates, compared to mean endogenous PPM1A expression (RQₘₑₐₙ=1).

TABLE 5A
PPMIA AON sequences (including PPMIA gapmer sequences and PPM1A
gapmer sequences with one or two spacers)

| | | | Internucleoside | | | |
|---|---|---|---|---|---|---|
| | | | Linkage | | | |
| | | | Pattern | | | |
| | | | (“s” indicates | | 500 nM | 200 nM |
| | | Spacer | phosphorothioate, | | Dose | Dose |
| SEQ ID | | Posi- | “o” indicates | | Relative | Relative |
| NO: | AON sequence 5′-3′* | tion | phosphodiester) | Gapmer structure | Quantity | Quantity |
| 148 | CCACTTGGCAAACCGATCAC | N/A | sssssssssssssssssss | eeeee-d10-eeeee | 0.171 | 0.287 |
| 148 | CCACTTGGCAAACCGATCAC | N/A | sososssssssssssosos | eeeee-d10-eeeee | 0.119 | 0.269 |
| 301692 | CCSCTTGGCAAACCGATCAC | 3 | sssssssssssssssssss | eeeee-d10-eeeee | 0.367 | 0.751 |
| 301693 | CCACSTGGCAAACCGATCAC | 5 | sssssssssssssssssss | eeeee-d10-eeeee | 0.380 | 0.580 |
| 301694 | CCACTTGGCAAACCGSTCAC | 16 | sssssssssssssssssss | eeeee-d10-eeeee | 0.246 | 0.375 |
| 301695 | CCACTTGSCAAACCGATCAC | 8 | sssssssssssssssssss | eeeee-d10-eeeee | 0.409 | 0.635 |
| 301696 | CCACTTGGCAAASCGATCAC | 13 | sssssssssssssssssss | eeeee-d10-eeeee | 0.342 | 0.515 |
| 301697 | CCACTTGGCASACCGATCAC | 11 | sssssssssssssssssss | eeeee-d10-eeeee | 0.319 | 0.474 |
| 301698 | CCACTTGGCAAACCGATCSC | 19 | sssssssssssssssssss | eeeee-d10-eeeee | 0.167 | 0.245 |
| 301699 | CCACTTGGCASACCGATCSC | 11 and 19 | sssssssssssssssssss | eeeee-d10-eeeee | 0.211 | 0.441 |
| 301700 | CCACTTSGCAAACCGATCAC | 7 | sssssssssssssssssss | eeeee-d10-eeeee | 0.164 | 0.292 |
| 301701 | SCACTTGGCAAACCGATCAC | 1 | sssssssssssssssssss | eeeee-d10-eeeee | 0.215 | 0.444 |
| 301702 | CSACTTGGCAAACCGATCAC | 2 | sssssssssssssssssss | eeeee-d10-eeeee | 0.160 | 0.384 |
| 301703 | CCASTTGGCAAACCGATCAC | 4 | sssssssssssssssssss | eeeee-d10-eeeee | 0.189 | 0.365 |
| 301704 | CCACTSGGCAAACCGATCAC | 6 | sssssssssssssssssss | eeeee-d10-eeeee | 0.101 | 0.254 |
| 301705 | CCACTTGGSAAACCGATCAC | 9 | sssssssssssssssssss | eeeee-d10-eeeee | 0.244 | 0.433 |
| 301706 | CCACTTGGCSAACCGATCAC | 10 | sssssssssssssssssss | eeeee-d10-eeeee | 0.262 | 0.377 |
| 301707 | CCACTTGGCAASCCGATCAC | 12 | sssssssssssssssssss | eeeee-d10-eeeee | 0.143 | 0.288 |
| 301708 | CCACTTGGCAAACSGATCAC | 14 | sssssssssssssssssss | eeeee-d10-eeeee | 0.091 | 0.228 |
| 301709 | CCACTTGGCAAACCSATCAC | 15 | sssssssssssssssssss | eeeee-d10-eeeee | 0.126 | 0.275 |
| 301710 | CCACTTGGCAAACCGASCAC | 17 | sssssssssssssssssss | eeeee-d10-eeeee | 0.063 | 0.185 |
| 301711 | CCACTTGGCAAACCGATSAC | 18 | sssssssssssssssssss | eeeee-d10-eeeee | 0.110 | 0.187 |
| 301712 | CCACTTGGCAAACCGATCAS | 20 | sssssssssssssssssss | eeeee-d10-eeeee | 0.041 | 0.117 |
| 186 | GCCCATCATACACAGCAAAG | N/A | sssssssssssssssssss | eeeee-d10-eeeee | 0.024 | 0.054 |
| 301713 | GCSCATCATACACAGCAAAG | 3 | sssssssssssssssssss | eeeee-d10-eeeee | 0.350 | 0.491 |
| 301714 | GCCCSTCATACACAGCAAAG | 5 | sssssssssssssssssss | eeeee-d10-eeeee | N/A | N/A |
| 301715 | GCCCATCATACACAGSAAAG | 16 | sssssssssssssssssss | eeeee-d10-eeeee | 0.101 | 0.195 |
| 301716 | GCCCATCSTACACAGCAAAG | 8 | sssssssssssssssssss | eeeee-d10-eeeee | 0.190 | 0.472 |
| 301717 | GCCCATCATACASAGCAAAG | 13 | sssssssssssssssssss | eeeee-d10-eeeee | 0.381 | 0.486 |
| 301718 | GCCCATCATASACAGCAAAG | 11 | sssssssssssssssssss | eeeee-d10-eeeee | 0.223 | 0.461 |
| 301719 | GCCCATCATACACAGCAASG | 19 | sssssssssssssssssss | eeeee-d10-eeeee | 0.091 | 0.168 |
| 301720 | GCCCATCSTACACAGSAAAG | 8 and 16 | sssssssssssssssssss | eeeee-d10-eeeee | 0.720 | 0.999 |
| 301721 | GCCCATCATASACAGCAASG | 11 and 19 | sssssssssssssssssss | eeeee-d10-eeeee | 0.330 | 0.565 |
| 301722 | GCCCATSATACACAGCAAAG | 7 | sssssssssssssssssss | eeeee-d10-eeeee | 0.083 | 0.214 |
| 301598 | AGTCCACTTGGCAAACCGATCAC | N/A | ssssssssssssssssssssss | eeeeee-d11-eeeeee | 0.077 | 0.219 |
| 301595 | TCCACTTGGCAAACCGATCACAG | N/A | ssssssssssssssssssssss | eeeeee-d11-eeeeee | 0.045 | 0.114 |
| 301723 | GTCCACTTGGCAAACCGATCACA | N/A | ssssssssssssssssssssss | eeeeee-d11-eeeeee | 0.066 | 0.158 |
| 301724 | CCACTTGGCAAACCGATCACAGC | N/A | ssssssssssssssssssssss | eeeeee-d11-eeeeee | 0.030 | 0.101 |
| 301725 | AGTCCACSTGGCAAASCGATCAC | 8 and 16 | ssssssssssssssssssssss | eeeeee-d11-eeeeee | 0.418 | 0.555 |
| 301726 | TCCACTTSGCAAACCSATCACAG | 8 and 16 | ssssssssssssssssssssss | eeeeee-d11-eeeeee | 0.486 | 0.651 |
| 301727 | GTCCACTSGGCAAACSGATCACA | 8 and 16 | ssssssssssssssssssssss | eeeeee-d11-eeeeee | 0.138 | 0.298 |
| 301728 | CCACTTGSCAAACCGSTCACAGC | 8 and 16 | ssssssssssssssssssssss | eeeeee-d11-eeeeee | 0.563 | 0.700 |

*Unless otherwise noted, for the sequences described in Table 5, the gapmer structure is denoted by “e” = a ribonucleoside and “d” = a deoxyribonucleoside.
Every ribonucleoside is a 2′-MOE modified ribonucleoside.
Each “C” cytosine that is a ribonucleoside is a 5-methylcytosine (5-MeC) (and therefore is a 5-MeC, 2′-MOE modified ribonucleoside).
Spacer, as indicated by S, is not a nucleoside.
S is a spacer represented by Formula (Iia′).

TABLE 5B
PPMIA AON sequences (including PPM1A gapmer sequences and PPMIA gapmer
sequences with one or two spacers)

| | | | Internucleoside | | | | | | |
|---|---|---|---|---|---|---|---|---|---|
| | | | Linkage Pattern | | | | | | |
| | | | (“s” indicates | | 500 nM | 200 nM | 50 nM | 20 nM | 5 nM |
| | AON | Spacer | phosphorothioate, | Gapmer | Dose | Dose | Dose | Dose | Dose |
| SEQ ID | sequence | Posi- | “o” indicates | struc- | Relative | Relative | Relative | Relative | Relative |
| NO: | 5′-3′* | tion | phosphodiester) | ture | Quantity | Quantity | Quantity | Quantity | Quantity |
| 301692 | CCSCTTGGCA | 3 | sosossssss | eeeee- | 0.364 | 0.444 | 0.688 | 0.764 | 1.070 |
| | AACCGATCAC | | sssssosos | d10- | | | | | |
| | | | | eeeee | | | | | |
| 301704 | CCACTSGGCA | 6 | sosossssss | eeeee- | 0.095 | 0.249 | 0.633 | 1.168 | N/A |
| | AACCGATCAC | | sssssosos | d10- | | | | | |
| | | | | eeeee | | | | | |
| 301694 | CCACTTGGCA | 16 | sosossssss | eeeee- | 0.193 | 0.176 | 0.356 | 0.546 | 0.940 |
| | AACCGSTCAC | | sssssosos | d10- | | | | | |
| | | | | eeeee | | | | | |
| 301695 | CCACTTGSCA | 8 | sosossssss | eeeee- | 0.292 | 0.561 | 0.672 | 1.063 | 1.010 |
| | AACCGATCAC | | sssssosos | d10- | | | | | |
| | | | | eeeee | | | | | |
| 301696 | CCACTTGGCA | 13 | sosossssss | eeeee- | 0.355 | 0.352 | 0.754 | 0.852 | 0.917 |
| | AASCGATCAC | | sssssosos | d10- | | | | | |
| | | | | eeeee | | | | | |
| 301697 | CCACTTGGCA | 11 | sosossssss | eeeee- | 0.153 | 0.325 | 0.517 | 0.736 | 0.745 |
| | SACCGATCAC | | sssssosos | d10- | | | | | |
| | | | | eeeee | | | | | |
| 301698 | CCACTTGGCA | 19 | sosossssss | eeeee- | 0.612 | 0.933 | 0.950 | 0.852 | 0.746 |
| | AACCGATCSC | | sssssosos | d10- | | | | | |
| | | | | eeeee | | | | | |
| 301742 | CCACTTGSCA | 8 | ssssssssss | eeeee- | 0.835 | 0.911 | 1.108 | 1.132 | 1.116 |
| | AACCGSTCAC | and | sssssssss | d10- | | | | | |
| | | 16 | | eeeee | | | | | |

* Unless otherwise noted, for the sequences described in Table 5, the gapmer structure is denoted by “e” = a ribonucleoside and “d” = a deoxyribonucleoside.
Every ribonucleoside is a 2′-MOE modified ribonucleoside.
Each “C” cytosine that is a ribonucleoside is a 5-methylcytosine (5-MeC) (and therefore is a 5-MeC, 2′-MOE modified ribonucleoside).
Spacer, as indicated by S, is not a nucleoside.
S is a spacer represented by Formula (Iia′).

### Example 2: Design and Selection of ATXN2 Antisense Oligonucleotides

ATXN2 antisense oligonucleotides were evaluated in iPSC derived human motor neurons (hMN) purchased from FujiFilm Cellular Dynamics (Catalog #: C1050). The cells were seeded in 96-well plates at a density of 10,000 cells/well. Antisense oligonucleotide (AON) to ATXN2 were transfected with Endoporter (Gene Tools, Philomath, OR, USA) to decrease expression of ATXN2 transcript. Vehicle control consisted of motor neuron treatment with Endoporter alone.

To evaluate ATXN2 AON ability to knock down ATXN2 mRNA transcripts, antisense oligonucleotides to ATXN2 were incubated with Endoporter in media before addition to the cells. After 72 hours, cells lysate was prepared and collected from the 96-well plates for RT-qPCR. From the cell lysates, cDNA was generated, and multiplexed RT-qPCR assay performed with Taqman probes for ATXN2 and reference GAPDH quantification.

Using the Applied Biosystems® PCR Thermal Cycler (SimpliAmp). cDNA was prepared directly from the cell lysates by performing one cycle of reverse transcription at 37° C. for 30 minutes, followed by heat inactivation of the RT enzyme at 95° C. for 5 minutes.

Transcript levels were detected by RT-qPCR using Taqman. Specifically, RT-qPCR was performed for detecting GAPDH using Thermofisher TaqMan Gene Expression Assay Hs_03929097_g1. RT-qPCR was performed for detecting ATXN2 using Thermofisher TaqMan Gene Expression Assay Hs00268077_m1.

RT-qPCR was performed on Applied Biosystems® QuantStudio 6 Flex Real-Time PCR System. One cycle of UNG incubation was performed at a temperature of 50° C. for 2 min. One cycle of polymerase activation/initial denaturation was performed at a temperature of 95° C. for 20 seconds. Forty cycles of amplification were performed at a temperature of 95° C. for 1 second followed by 60° C. for 20 seconds.

ATXN2 signal cycle threshold (Ct) was normalized to GAPDH (ΔCt). To visualize the quantitative changes (e.g., ratio of ATXN2 transcript decrease), the normalized ATXN2 signal was further normalized to the mean vehicle signal (treated with Endoporter alone, ΔΔCt). Relative quantity (RQ) of transcript level was calculated using the equation RQ=2^{-ΔΔCt} and is used to describe the treatment condition comparison to normal, healthy levels.

ATXN2 AON knock-down of ATXN2 transcripts were presented as mean relative quantity (RQₘₑₐₙ) of the technical triplicates, compared to mean endogenous ATXN2 expression (RQₘₑₐₙ=1).

TABLE 6
ATXN2 AON sequences (including ATXN2 gapmer sequences and
ATXN2 gapmer sequences with one or two spacers)

| | | | Internucleoside | | | |
|---|---|---|---|---|---|---|
| | | | Linkage Pattern | | | |
| | | | (“s” indicates | | 500 nM | 200 nM |
| SEQ | | | phosphorothioate, | | Dose | Dose |
| ID | | Spacer | “o” indicates | | Relative | Relative |
| NO: | AON sequence 5′-3′* | Position | phosphodiester) | Gapmer structure | Quantity | Quantity |
| 301601 | GCTAACTGGTTTGCCCTTGC | N/A | soooossssssssssooss | eeeee-d10-eeeee | 0.258 | 0.440 |
| 301602 | GTGGGATACAAATTCTAGGC | N/A | soooossssssssssooss | eeeee-d10-eeeee | 0.066 | 0.240 |
| 301603 | GGAGCTGGAGAACCATGAGC | N/A | sssssssssssssssssss | eeeee-d10-eeeee | 0.754 | 0.693 |
| 301604 | TGTACTTCACATTTGGAGCC | N/A | sssssssssssssssssss | eeeeee-d10-eeee | 0.416 | 0.642 |
| 301729 | GCTAACTGGTSTGCCCTTGC | 11 | soooossssssssssooss | eeeee-d10-eeeee | 0.455 | 0.693 |
| 301730 | GTGGGATACASATTCTAGGC | 11 | soooossssssssssooss | eeeee-d10-eeeee | 0.170 | 0.473 |
| 301731 | GGAGCTGGAGSACCATGAGC | 11 | sssssssssssssssssss | eeeee-d10-eeeee | 0.654 | 0.773 |
| 301732 | TGTACTTCACSTTTGGAGCC | 11 | sssssssssssssssssss | eeeeee-d10-eeee | 0.911 | 1.280 |

*Unless otherwise noted, for the sequences described in Table 6, the gapmer structure is denoted by “e” = a ribonucleoside and “d” = a deoxyribonucleoside.
Every ribonucleoside is a 2′-MOE modified ribonucleoside.
Each “C” cytosine is a 5-methylcytosine (5-MeC).
Spacer, as indicated by S, is not a nucleoside.
S is a spacer represented by Formula (Iia′).

### Example 3: Design and Selection of SOD1 Antisense Oligonucleotides

SOD1 antisense oligonucleotides were evaluated in iPSC derived human motor neurons (hMN). The cells were seeded in 96-well plates at a density of 20,000 cells/well. Antisense oligonucleotide (AON) to SOD1 were transfected with Endoporter (Gene Tools, Philomath, OR, USA) to decrease expression of SOD1 transcript. Vehicle control consisted of motor neuron treatment with Endoporter alone.

To evaluate SOD1 AON ability to knock down SOD1 mRNA transcripts, antisense oligonucleotides to SOD1 were incubated with Endoporter in media before addition to the cells. After 72 hours, cells lysate was prepared and collected from the 96-well plates for RT-qPCR. From the cell lysates, cDNA was generated, and multiplexed RT-qPCR assay performed with Taqman probes for SOD1 and reference GAPDH quantification.

Transcript levels were detected by RT-qPCR using Taqman. Specifically, RT-qPCR was performed for detecting GAPDH using Thermofisher TaqMan Gene Expression Assay Hs_03929097_g1. RT-qPCR was performed for detecting SOD1 using Thermofisher TaqMan Gene Expression Assay Hs00533490_m1.

RT-qPCR was performed on Applied Biosystems®7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

SOD1 signal cycle threshold (Ct) was normalized to GAPDH (ΔCt). To visualize the quantitative changes (e.g., ratio of SOD1 transcript decrease), the normalized SOD1 signal was further normalized to the mean vehicle signal (treated with Endoporter alone, ΔΔCt). Relative quantity (RQ) of transcript level was calculated using the equation RQ=2^{-ΔΔCt} and is used to describe the treatment condition comparison to normal, healthy levels.

SOD1 AON knock-down of SOD1 transcripts were presented as mean relative quantity (RQₘₑₐₙ) of the technical triplicates, compared to mean endogenous SOD1 expression (RQₘₑₐₙ=1).

TABLE 7
SOD1 AON sequences (including SOD1 gapmer sequences and
SOD1 gapmer sequences with one or two spacers)

| | | | Internucleoside | | | |
|---|---|---|---|---|---|---|
| | | | Linkage | | | |
| | | | Pattern | | | |
| | | | (“s” indicates | | 500 nM | 200 nM |
| SEQ | AON sequence 5′-3′ | | phosphorothioate, | | Dose | Dose |
| ID | S indicates | Spacer | “o” indicates | | Relative | Relative |
| NO: | presence of spacer | Position | phosphodiester) | Gapmer structure | Quantity | Quantity |
| 301605 | CAGGATACATTTCTACAGCT | N/A | sososssssssssssosos | eeeee-d10-eeeee | 0.019 | 0.024 |
| 301733 | CAGGSTACATTTCTACAGCT | 5 | sososssssssssssosos | eeeee-d10-eeeee | 0.124 | 0.159 |
| 301734 | CAGGATACATTTCTACSGCT | 17 | sososssssssssssosos | eeeee-d10-eeeee | 0.117 | 0.179 |
| 301735 | CAGGATACATSTCTACAGCT | 11 | sososssssssssssosos | eeeee-d10-eeeee | 0.021 | 0.051 |
| 301736 | CAGGATSCATTTCTSCAGCT | 7 and 15 | sososssssssssssosos | eeeee-d10-eeeee | 0.393 | 0.582 |
| 301737 | CAGGSTACATTTCTACSGCT | 5 and 17 | sososssssssssssosos | eeeee-d10-eeeee | 1.150 | 1.070 |

* Unless otherwise noted, for the sequences described in Table 7, the gapmer structure is denoted by “e” = a ribonucleoside and “d” = a deoxyribonucleoside.
Every ribonucleoside is a 2′-MOE modified ribonucleoside. Each “C” cytosine is a 5-methylcytosine (5-MeC).
Spacer, as indicated by S, is not a nucleoside.
S is a spacer represented by Formula (lia′).

### Example 4: Design and Selection of MAPT Antisense Oligonucleotides

MAPT antisense oligonucleotides were evaluated in iPSC derived human motor neurons (hMN) purchased from FujiFilm Cellular Dynamics (Catalog #: C1050). The cells were seeded in 96-well plates at a density of 10,000 cells/well. Antisense oligonucleotide (AON) to MAPT were transfected with Endoporter (Gene Tools, Philomath, OR, USA) to decrease expression of MAPT transcript. Vehicle control consisted of motor neuron treatment with Endoporter alone.

To evaluate MAPT AON ability to knock down MAPT mRNA transcripts, antisense oligonucleotides to MAPT were incubated with Endoporter in media before addition to the cells. After 72 hours, cells lysate was prepared and collected from the 96-well plates for RT-qPCR. From the cell lysates, cDNA was generated, and multiplexed RT-qPCR assay performed with Taqman probes for MAPT and reference GAPDH quantification.

Using the Applied Biosystems® QuantStudio 6 Flex Real-Time PCR System, cDNA was prepared directly from the cell lysates by performing one cycle of reverse transcription at 37° C. for 30 minutes, followed by heat inactivation of the RT enzyme at 95° C. for 5 minutes.

Transcript levels were detected by RT-qPCR using Taqman. Specifically, RT-qPCR was performed for detecting GAPDH using Thermofisher TaqMan Gene Expression Assay Hs_03929097_g1. RT-qPCR was performed for detecting MAPT using Thermofisher TaqMan Gene Expression Assay Hs00902194_m1.

RT-qPCR was performed on Applied Biosystems® QuantStudio 6 Flex Real-Time PCR System. One cycle of UNG incubation was performed at a temperature of 50° C. for 2 min. One cycle of polymerase activation/initial denaturation was performed at a temperature of 95° C. for 20 seconds. Forty cycles of amplification were performed at a temperature of 95° C. for 1 second followed by 60° C. for 20 seconds.

MAPT signal cycle threshold (Ct) was normalized to GAPDH (ΔCt). To visualize the quantitative changes (e.g., ratio of MAPT transcript decrease), the normalized MAPT signal was further normalized to the mean vehicle signal (treated with Endoporter alone, ΔΔCt). Relative quantity (RQ) of transcript level was calculated using the equation RQ=2−ΔΔCt and is used to describe the treatment condition comparison to normal, healthy levels.

MAPT AON knock-down of MAPT transcripts were presented as mean relative quantity (RQₘₑₐₙ) of the technical triplicates, compared to mean endogenous MAPT expression (RQₘₑₐₙ=1).

TABLE 8
MAPT AON sequences (including MAPT gapmer sequences and
MAPT gapmer sequences with one or two spacers)

| | | | Internucleoside | | | |
|---|---|---|---|---|---|---|
| | | | Linkage Pattern | | | |
| | AON sequence 5′-3′ | | (“s” indicates | | 500 nM | 200 nM |
| SEQ | S indicates | Spacer | phosphorothioate, | | Dose | Dose |
| ID | presence | Posi- | “o” indicates | | Relative | Relative |
| NO: | of spacer | tion | phosphodiester) | Gapmer structure | Quantity | Quantity |
| 301606 | CCGTTTTCTTACCACCCT | N/A | sossssssssssssoss | eeeee-d8-eeeee | 0.230 | 0.407 |
| 301607 | CCGTTTTCTTACCACCCTAA | N/A | sosssssssssssssosss | eeeee-d10-eeeee | 0.138 | 0.270 |
| 301738 | CCGTTTTCTTSCCACCCT | 11 | sossssssssssssoss | eeeee-d8-eeeee | 1.037 | 1.283 |
| 301739 | CCGTTTTCSTACCACCCT | 9 | sossssssssssssoss | eeeee-d8-eeeee | 0.841 | 1.117 |
| 301740 | CCGTTTTCTSACCACCCT | 10 | sossssssssssssoss | eeeee-d8-eeeee | 1.007 | 1.215 |
| 301741 | CCGTTTTCTTSCCACCCTAA | 11 | sosssssssssssssosss | eeeee-d10-eeeee | 0.141 | 0.420 |

* Unless otherwise noted, for the sequences described in Table 8, the gapmer structure is denoted by “e” = a ribonucleoside and “d” = a deoxyribonucleoside.
Every ribonucleoside is a 2′-MOE modified ribonucleoside.
Each “C” cytosine is a 5-methylcytosine (5-MeC).
Spacer, as indicated by S, is not a nucleoside.
S is a spacer represented by Formula (Iia′).

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

Lengthy table referenced here
US20260258423A1-20260903-T00001
Please refer to the end of the specification for access instructions.

Lengthy table referenced here
US20260258423A1-20260903-T00002
Please refer to the end of the specification for access instructions.

Lengthy table referenced here
US20260258423A1-20260903-T00003
Please refer to the end of the specification for access instructions.

Lengthy table referenced here
US20260258423A1-20260903-T00004
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260258423A1). An electronic copy of the table will also be available from the USPTO upon request and payment of the fee set forth in 37 CFR 1.19(b)(3).

What is claimed is:

1. A compound comprising a gapmer oligonucleotide, and wherein the gapmer oligonucleotide comprises a spacer.

2. A gapmer oligonucleotide, wherein the gapmer oligonucleotide comprises a spacer.

3. The compound or gapmer oligonucleotide of claim 1 or 2, wherein the gapmer oligonucleotide comprises a second spacer that is non-adjacent to the spacer.

4. The compound or gapmer oligonucleotide of any one of claims 1-3, wherein the gapmer oligonucleotide comprises a sequence that is between 85 and 98% complementary to an equal length portion of a transcript whose presence leads to a neurological disease.

5. The compound or gapmer oligonucleotide of any one of claims 1-3, wherein the gapmer oligonucleotide comprises a sequence that is between 85 and 98% complementary to an equal length portion of a PPM1A mRNA or pre-mRNA transcript, an ATXN2 mRNA or pre-mRNA transcript, a SOD1 mRNA or pre-mRNA transcript, or a MAPT mRNA or pre-mRNA transcript.

6. The compound or gapmer oligonucleotide of any one of claims 1-5, wherein the gapmer oligonucleotide comprises a sequence that is between 85 and 98% complementary to an equal length portion of any one of SEQ ID NOs: 1909-1913, 149355-149361, 167802-167804, or 301567-301589, a sequence having 90% identity thereof, or to a 15 to 50 contiguous nucleobase portion thereof.

7. The compound or gapmer oligonucleotide of any one of claims 1-6, wherein the gapmer oligonucleotide comprises a sequence that shares at least 85% identity with an equal length portion of any one of SEQ ID NOs: 1-954, 1914-149354, 149362-158581, 167805-301566, SEQ ID NOs: 301692-301742 or the sequences in Tables 4A-4C.

8. The compound or gapmer oligonucleotide of any one of claims 1-7, wherein the gapmer oligonucleotide comprises a sequence that shares at least 90% identity with an equal length portion of any one of SEQ ID NOs: 1-954, 1914-149354, 149362-158581, 167805-301566, SEQ ID NOs: 301692-301742 or the sequences in Tables 4A-4C.

9. The compound or gapmer oligonucleotide of any one of claims 1-7, wherein the gapmer oligonucleotide comprises a sequence that shares at least 95% identity with an equal length portion of any one of SEQ ID NOs: 1-954, 1914-149354, 149362-158581, 167805-301566, SEQ ID NOs: 301692-301742 or the sequences in Tables 4A-4C.

10. The compound or gapmer oligonucleotide of any one of claims 1-8, wherein the gapmer oligonucleotide comprises a sequence that shares at least 100% identity with an equal length portion of any one of SEQ ID NOs: 1-954, 1914-149354, 149362-158581, 167805-301566, SEQ ID NOs: 301692-301742 or the sequences in Tables 4A-4C.

11. The compound or gapmer oligonucleotide of any one of claims 1-9, wherein the gapmer oligonucleotide comprises a segment with at most 11 linked nucleosides, and wherein the gapmer oligonucleotide comprises a sequence that shares at least 85% identity with an equal length portion of any one of SEQ ID NOs: 1-954, 1914-149354, 149362-158581, 167805-301566, SEQ ID NOs: 301692-301742 or the sequences in Tables 4A-4C.

12. The compound or gapmer oligonucleotide of any one of claims 1-10, wherein the gapmer oligonucleotide comprises a segment with at most 10, 9, 8, 7, 6, 5, 4, 3, or 2 linked nucleosides, and wherein the gapmer oligonucleotide comprises a sequence that shares at least 85% identity with an equal length portion of any one of SEQ ID NOs: 301692-301742 or the sequences in Tables 4A-4C.

13. The compound or gapmer oligonucleotide of any one of claims 1-10, wherein the gapmer oligonucleotide comprises a segment with at most 11 linked nucleosides, and wherein the gapmer oligonucleotide comprises a sequence that shares at least 90% identity with an equal length portion of any one of SEQ ID NOs: 301692-301742 or the sequences in Tables 4A-4C.

14. The compound or gapmer oligonucleotide of any one of claims 1-10, wherein the gapmer oligonucleotide comprises a segment with at most 10, 9, 8, 7, 6, 5, 4, 3, or 2 linked nucleosides, and wherein the gapmer oligonucleotide comprises a sequence that shares at least 90% identity with an equal length portion of any one of SEQ ID NOs: 301610-301741.

15. The compound or gapmer oligonucleotide of any one of claims 1-14, wherein the gapmer oligonucleotide is at least 18, at least 19, at least 20, at least 21, at least 22, at least 23, at least 24, or at least 25 oligonucleotide units in length.

16. The compound or gapmer oligonucleotide of any one of claims 1-15, wherein at least one (i.e., one or more) nucleoside linkage of the gapmer oligonucleotide is a non-natural linkage,

17. The compound or gapmer oligonucleotide of any one of claims 1-16, wherein the gapmer oligonucleotide is at least 19 oligonucleotide units in length.

18. The compound of oligonucleotide of any one of claims 1-17, wherein the spacer is a nucleoside-replacement group comprising a non-sugar substitute that is incapable of linking to a nucleotide base.

19. The compound or gapmer oligonucleotide of claim 18, wherein the spacer is located between positions 5 and 11 of the gapmer oligonucleotide.

20. The compound or gapmer oligonucleotide of claim 18, wherein the spacer is located between positions 7 and 11 of the gapmer oligonucleotide.

21. The compound or gapmer oligonucleotide of claim 18, wherein the gapmer oligonucleotide further comprises a second spacer, and wherein the spacer and the second spacer are non-adjacent to one another.

22. The compound or gapmer oligonucleotide of claim 21, wherein the gapmer oligonucleotide further comprises a second spacer, wherein the second spacer is located between positions 15 and 19 of the gapmer oligonucleotide.

23. The compound or gapmer oligonucleotide of claim 22, wherein the spacer and the second spacer are separated by at least 5 nucleobases, at least 6 nucleobases, or at least 7 nucleobases in the gapmer oligonucleotide.

24. The compound or gapmer oligonucleotide of claim 22 or 23, wherein the spacer is located between positions 5 and 11 of the gapmer oligonucleotide, and wherein the second spacer is located between positions 15 and 19 of the gapmer oligonucleotide.

25. The compound or gapmer oligonucleotide of any one of claims 22-24, wherein the spacer is located at position 8 of the gapmer oligonucleotide, and wherein the second spacer is located at position 16 of the gapmer oligonucleotide.

26. The compound or gapmer oligonucleotide of any one of claims 22-24, wherein the spacer is located at position 5 of the gapmer oligonucleotide, and wherein the second spacer is located at position 17 of the gapmer oligonucleotide.

27. The compound or gapmer oligonucleotide of any one of claims 22-24, wherein the spacer is located at position 7 of the gapmer oligonucleotide, and wherein the second spacer is located at position 15 of the gapmer oligonucleotide.

28. The compound or gapmer oligonucleotide of any one of claims 22-24, wherein the spacer is located at position 11 of the gapmer oligonucleotide, and wherein the second spacer is located at position 19 of the gapmer oligonucleotide.

29. The compound or gapmer oligonucleotide of any one of claims 18-28, wherein each of the spacer or second spacer is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

30. The compound or gapmer oligonucleotide of any one of claims 18-29, wherein each of the spacer or second spacer is independently represented by Formula (X), wherein: 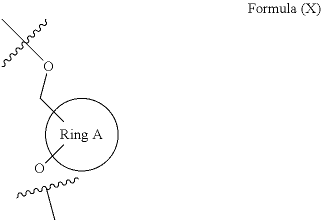
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 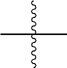
symbol represents the point of connection to an internucleoside linkage.

31. The compound or gapmer oligonucleotide of claim 30, wherein each of the spacer or second spacer is independently represented by Formula (Xa), wherein:

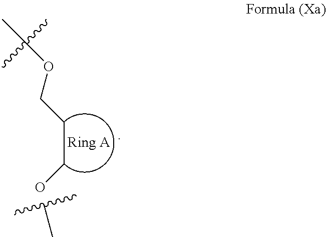

32. The compound or nucleotide of claim 30 or 31, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrrolidinyl, piperidinyl, piperazinyl, morpholinyl and azepanyl.

33. The compound or nucleotide of claim 32 wherein ring A is tetrahydrofuranyl.

34. The compound or nucleotide of claim 32 wherein ring A is tetrahydropyranyl.

35. The compound or gapmer oligonucleotide of any one of claims 18-29, wherein each of the spacer and second spacer is independently represented by Formula I, wherein: 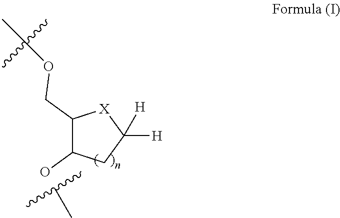
X is selected from —CH₂— and —O—; and
n is 0, 1, 2 or 3.

36. The compound or gapmer oligonucleotide of any one of claims 18-29, wherein each of the spacer and second spacer is independently represented by Formula I′, wherein: 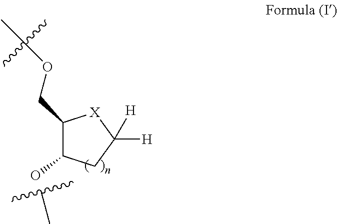
X is selected from —CH₂— and —O—; and
n is 0, 1, 2 or 3.

37. The compound or gapmer oligonucleotide of any one of claims 18-29, wherein each of the spacer and second spacer is independently represented by Formula (Ia), wherein: 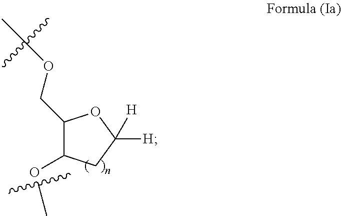
and
n is 0, 1, 2 or 3.

38. The compound or gapmer oligonucleotide of any one of claims 18-29, wherein each of the spacer and second spacer is independently represented by Formula (Ia′), wherein: 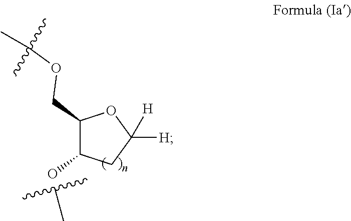
and
n is 0, 1, 2 or 3.

39. The compound or gapmer oligonucleotide of any one of claims 18-29, wherein each of the spacer and second spacer is independently represented by Formula II, wherein: 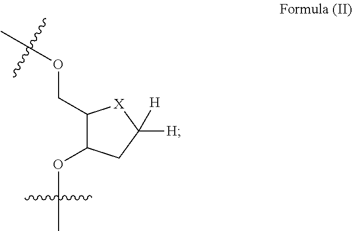
and
X is selected from —CH₂— and —O—.

40. The compound or gapmer oligonucleotide of any one of claims 18-29, wherein each of the spacer and second spacer is independently represented by Formula II′, wherein: 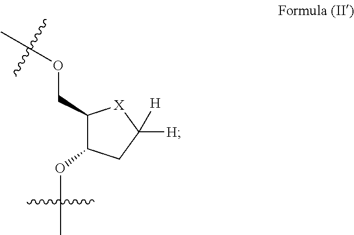
and
X is selected from —CH₂— and —O—.

41. The compound or gapmer oligonucleotide of any one of claims 18-29, wherein each of the spacer and second spacer is independently represented by Formula (Iia), wherein:

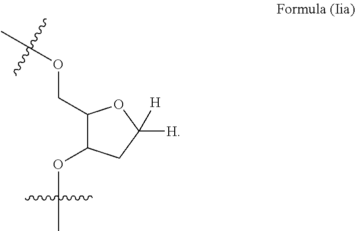

42. The compound or gapmer oligonucleotide of any one of claims 18-29, wherein each of the spacer and second spacer is independently represented by Formula (Iia′), wherein:

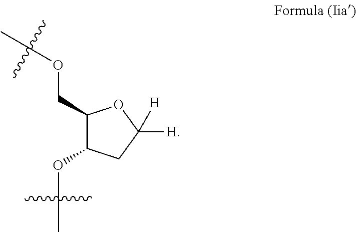

43. The compound or gapmer oligonucleotide of any one of claims 18-29, wherein each of the spacer and second spacer is independently is independently represented by Formula III, wherein: 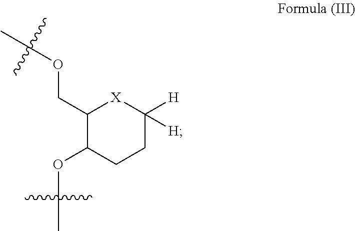
and
X is selected from —CH₂— and —O—.

44. The compound or gapmer oligonucleotide of any one of claims 18-29, wherein each of the spacer and second spacer is independently represented by Formula III′, wherein: 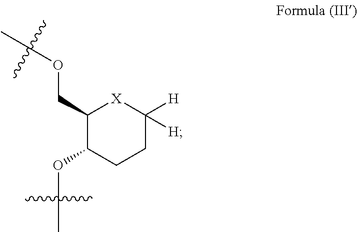
and
X is selected from —CH₂— and —O—.

45. The compound or gapmer oligonucleotide of any one of claims 18-29, wherein each of the spacer and second spacer is independently represented by Formula (IIIa), wherein:

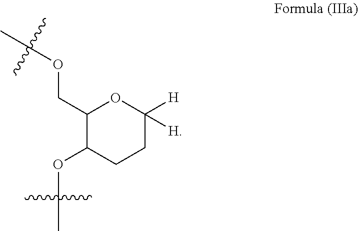

46. The compound or gapmer oligonucleotide of any one of claims 18-29, wherein each of the spacer and second spacer is independently represented by Formula (IIIa′), wherein:

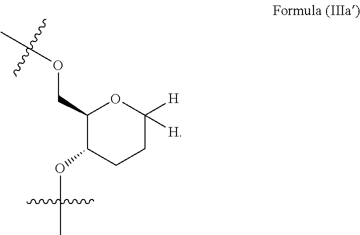

47. The compound or gapmer oligonucleotide of any one of the above claims, wherein the gapmer oligonucleotide comprising the spacer has a GC content of at least 10%.

48. The compound or gapmer oligonucleotide of any one of the above claims, wherein the gapmer oligonucleotide comprising the spacer has a GC content of at least 20%.

49. The compound or gapmer oligonucleotide of any one of the above claims, wherein the gapmer oligonucleotide comprising the spacer has a GC content of at least 25%.

50. The compound or gapmer oligonucleotide of any one of the above claims, wherein the gapmer oligonucleotide comprising the spacer has a GC content of at least 30%.

51. The compound or gapmer oligonucleotide of any one of the above claims, wherein the gapmer oligonucleotide comprising the spacer has a GC content of at least 40%.

52. The compound or gapmer oligonucleotide of any one of the above claims, wherein the gapmer oligonucleotide comprising the spacer has a GC content of at least 50%.

53. The compound or gapmer oligonucleotide of any one of the above claims, wherein at least one (i.e., one or more) nucleoside linkage of the gapmer oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

54. The gapmer oligonucleotide of any one of the preceding claims, wherein at least one internucleoside linkage of the nucleotide sequence is a phosphorothioate linkage.

55. The gapmer oligonucleotide of claim 54, wherein the phosphorothioate internucleoside linkage is in one of a Rp configuration or a Sp configuration.

56. The gapmer oligonucleotide of any one of claims 1-55, wherein the gapmer oligonucleotide comprises one or more chiral centers and/or double bonds.

57. The gapmer oligonucleotide of claim 55, wherein the gapmer oligonucleotide exists as stereoisomers selected from geometric isomers, enantiomers, and diastereomers.

58. The gapmer oligonucleotide of any one of the preceding claims, wherein all internucleoside linkages of the nucleotide sequence are phosphorothioate linkages.

59. The gapmer oligonucleotide of any one of the preceding claims, wherein the gapmer oligonucleotide comprises at least one modified nucleobase.

60. The gapmer oligonucleotide of claim 59, wherein the at least one modified nucleobase is 5-methylcytosine, pseudouridine, or 5-methoxyuridine.

61. The gapmer oligonucleotide of any one of the preceding claims, wherein the gapmer oligonucleotide comprises at least one nucleoside with a modified sugar moiety.

62. The gapmer oligonucleotide of claim 61, wherein the modified sugar moiety is one of a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

63. The gapmer oligonucleotide of any one of the preceding claims, wherein the gapmer oligonucleotide comprises two, three, four, five, six, seven, eight, nine, or ten nucleosides with modified sugar moieties.

64. The gapmer oligonucleotide of claim 63, wherein the modified sugar moieties are independently any one of a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-D-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

65. The gapmer oligonucleotide of any one of the preceding claims, wherein the gapmer oligonucleotide comprises ten 2′-O-(2-methoxyethyl) (2′-MOE) nucleosides.

66. The gapmer oligonucleotide of claim 65, wherein five of the 2′-O-(2-methoxyethyl) (2′-MOE) nucleosides are located at the 3′ end of the gapmer oligonucleotide, and wherein five of the 2′-O-(2-methoxyethyl) (2′-MOE) nucleosides are located at the 5′ end of the gapmer oligonucleotide.

67. The gapmer oligonucleotide of claim 65, wherein the modified sugar moeities are locked nucleic acids (LNAs), and wherein two or more of the nucleosides of the 3′ wing region of the gapmer oligonucleotide are LNAs, and wherein two or more of the nucleosides of the 5′ wing region of the gapmer oligonucleotide.

68. The gapmer oligonucleotide of any one of claims 61-66, wherein the at least one nucleoside with the modified sugar moiety or the nucleosides with modified sugar moieties are ribonucleosides.

69. The gapmer oligonucleotide of any one of the preceding claims, wherein the gapmer oligonucleotide comprises at least one deoxyribonucleoside.

70. The gapmer oligonucleotide of any one of the preceding claims, wherein the gapmer oligonucleotide comprises two, three, four, five, six, seven, eight, nine, or ten deoxyribonucleosides.

71. The gapmer oligonucleotide of any one of claims 1-53, wherein the gapmer oligonucleotide comprises:
a gap segment comprising one or more of linked deoxyribonucleosides, 2′-Fluoro Arabino Nucleic Acids (FANA), and Fluoro Cyclohexenyl nucleic acid (F-CeNA);
a 5′ wing region comprising linked nucleosides; and
a 3′ wing region comprising linked nucleosides;
wherein the central region comprises a region of at least 8 oligonucleotide units comprising at least 4 contiguous nucleobases, the region having at least 80% identity to an equal length portion of any one of SEQ ID NOs: 1-954, 1914-149354, 149362-158581, 167805-301566, SEQ ID NOs: 301692-301742 or the sequences in Tables 4A-4C positioned between the 5′ wing segment and the 3′ wing segment; wherein the 5′ wing region and the 3′ wing region each comprises at least two linked nucleosides; and wherein at least one nucleoside of each wing region comprises a modified sugar.

72. The gapmer oligonucleotide of claim 71, wherein the at least two linked nucleosides of the 5′ wing region are linked through a phosphorothioate internucleoside linkage and/or wherein the at least two linked nucleosides of the 3′ wing region are independently linked through a phosphorothioate internucleoside linkage.

73. The gapmer oligonucleotide of claim 71 or 72, wherein every internucleoside linkage of the 5′ wing region and/or every internucleoside linkage of the 3′ wing region, independently are phosphorothioate internucleoside linkages.

74. The gapmer oligonucleotide of claim 71 or 72, wherein the 5′ wing region further comprises at least one phosphodiester internucleoside linkage.

75. The gapmer oligonucleotide of claim 71 or 72, wherein the 3′ wing region further comprises at least one phosphodiester internucleoside linkage.

76. The gapmer oligonucleotide of claim 71, wherein the at least two linked nucleosides of the 5′ wing region are linked through a phosphodiester internucleoside linkage and/or wherein the at least two linked nucleosides of the 3′ wing region are independently linked through a phosphodiester internucleoside linkage.

77. The gapmer oligonucleotide of any one of claims 71-76, wherein at least one of the internucleoside linkages of the central region is a phosphodiester linkage.

78. The gapmer oligonucleotide of claim 77, wherein at least two, at least three, at least four, at least five, at least six, at least seven, at least eight, or at least nine of the internucleoside linkages of the central region are phosphodiester linkages.

79. The gapmer oligonucleotide of any one of claims 71-76, wherein at least one of the internucleoside linkages of the central region is a phosphorothioate internucleoside linkage.

80. The gapmer oligonucleotide of claim 79, wherein at least two, at least three, at least four, at least five, at least six, at least seven, at least eight, or at least nine of the internucleoside linkages of the central region are phosphorothioate internucleoside linkages.

81. The gapmer oligonucleotide of any one of claims 71-72 or 79-80, wherein all internucleoside linkages of the gapmer oligonucleotide are phosphorothioate internucleoside linkages.

82. The gapmer oligonucleotide of any one of claims 71-81, wherein any one or all of the phosphorothioate internucleoside linkages are in a Rp configuration, a Sp configuration, or in any combination of Rp and Sp configuration.

83. The gapmer oligonucleotide of any one of claims 71-82, wherein the gapmer oligonucleotide comprises at least one modified sugar moiety.

84. The gapmer oligonucleotide of claim 83, wherein the 5′ wing region or the 3′ wing region comprises the at least one modified sugar moiety.

85. The gapmer oligonucleotide of claim 83, wherein the central region comprises the at least one modified sugar moiety.

86. The gapmer oligonucleotide of any one of claims 83-85, wherein the at least one modified sugar moiety is any one of a 2′-OMe modified sugar moiety, bicyclic sugar moiety, 2′-O-(2-methoxyethyl) (2′-MOE), 2′-O—(N-methylacetamide), 2′-deoxy-2′-fluoro nucleoside, 2′-fluoro-D-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

87. The gapmer oligonucleotide of any one of claims 83-86 wherein the gapmer oligonucleotide comprises one or more 2′-MOE nucleosides.

88. The gapmer oligonucleotide of claim 87, wherein the 5′ wing region or the 3′ wing region comprise one or more 2′-MOE nucleosides.

89. The gapmer oligonucleotide of claim 87 or 88, wherein the 5′ wing region or the 3′ wing region comprise two, three, four, five, or six 2′-MOE nucleosides.

90. The gapmer oligonucleotide of claim 89, wherein every nucleoside of the 5′ wing region or the 3′ wing region is a 2′-MOE nucleoside.

91. The gapmer oligonucleotide of claim 87, wherein the central region comprises one or more 2′-MOE nucleosides.

92. The gapmer oligonucleotide of claim 91, wherein the central region comprises two, three, four, five, six, seven, eight, nine, or ten 2′-MOE nucleosides.

93. The gapmer oligonucleotide of claim 92, wherein every nucleoside of the central region is a 2′-MOE nucleoside.

94. The gapmer oligonucleotide of any one of claims 87-93, wherein the one or more 2′-MOE nucleosides are linked through phosphorothioate internucleoside linkages.

95. The gapmer oligonucleotide of claim 71, wherein the gapmer oligonucleotide comprises sugar modifications in any of the following patterns: eeeee-d10-eeeee, eeeee-d8-eeeee, eeeeee-d11-eeeeee, eee-d8-eee, eee-d10-eee, eeee-d10-eeee, eeeeee-d10-eeee, and eeee-d8-eeee, wherein e=2′-MOE nucleoside and d=a deoxyribonucleoside, and wherein at least one “e” or at least one “d” is replaced with a spacer.

96. The gapmer oligonucleotide of claim 95, wherein the gapmer oligonucleotide comprises internucleoside linkages in any of the following patterns: sssssooooooooosssss; ooooosssssssssooooo; oooooooooooooosssss; soossssssssssssssss; sososssssssssssosos; ssssssssssssssssoos; sssssoooooooooooooo; sssssssssssssssssss; ssssssssssssssssssssss; sssooooooosss; ooosssssssooo; sssssssssssss; sosssssssssos; sosssssssssss; sssssssssssos; ssssssssssooo; ooossssssssss; sssooooooooosss; ooosssssssssooo; sssssssssssssss; ssssssssssssooo; ooossssssssssss; sosssssssssssos; sosssssssssssss; sssssssssssssos; ssssooooooooossss; oooosssssssssoooo; sssssssssssssssss; sssssssssssssoooo; soosssssssssssoos; soossssssssssssss; ssssssssssssssoos; oooosssssssssssss; ssssooooooossss; oooosssssssoooo; sssssssssssoooo; oooosssssssssss; soosssssssssoos; soossssssssssss; ssssssssssssoos; soooossssssssssooos; soooossssssssssooss; sossssssssssssoss; sosssssssssssssosss; or sssssssssssssss; wherein s=a phosphorothioate linkage, and o=a phosphodiester linkage.

97. The gapmer oligonucleotide of claim 95 or 96, wherein the gapmer oligonucleotide comprises sugar modification and internucleoside linkage combinations, respectively, in any of the following patterns:
a) eeeee-d10-eeeee and sssssooooooooosssss;
b) eeeee-d10-eeeee and ooooosssssssssooooo;
c) eeeee-d10-eeeee and sssssssssssssssssss;
d) eee-d8-eee and sssooooooosss;
e) eee-d8-eee and ooosssssssooo
f) eee-d8-eee and sssssssssssss;
g) eee-d10-eee and sssooooooooosss;
h) eee-d10-eee and ooosssssssssooo;
i) eee-d10-eee and sssssssssssssss;
j) eeee-d10-eeee and ssssooooooooossss;
k) eeee-d10-eeee and oooosssssssssoooo;
l) eeee-d10-eeee and sssssssssssssssss;
m) eeee-d8-eeee and ssssooooooossss,
n) eeee-d8-eeee and oooosssssssoooo,
o) eeee-d8-eeee and sssssssssssssss,
p) eeeeee-d11-eeeeee and ssssssssssssssssssssss;
q) eeeee-d10-eeeee and sososssssssssssosos;
r) eeeee-d10-eeeee and soooossssssssssooos;
s) eeeee-d10-eeeee and soooossssssssssooss;
t) eeeee-d8-eeeee and sossssssssssssoss;
u) eeeee-d10-eeeee and sosssssssssssssosss;
v) eeeeee-d10-eeee and sssssssssssssssssss;
wherein e=2′-MOE nucleoside and d=a deoxyribonucleoside, and wherein s=a phosphorothioate linkage, and o=a phosphodiester linkage, and wherein at least one “e” or at least one “d” of the gapmer oligonucleotide is replaced with a spacer.

98. The gapmer oligonucleotide of any one of claims 71-97, wherein the gapmer oligonucleotide comprises at least one modified nucleobase.

99. The gapmer oligonucleotide of claim 98, wherein the 5′ wing region or the 3′ wing region comprises the at least one modified nucleobase.

100. The gapmer oligonucleotide of claim 98, wherein the central region comprises the at least one modified nucleobase.

101. The gapmer oligonucleotide of any one of claims 98-100, wherein the at least one modified nucleobase is 5-methylcytosine, pseudouridine, or 5-methoxyuridine.

102. The gapmer oligonucleotide of any one of claims 98-101, wherein every cytosine in the 5′ wing region or the 3′ wing region is a 5-methylcytosine.

103. The gapmer oligonucleotide of any one of claims 98-102, wherein every cytosine in the central region is a 5-methylcytosine.

104. The gapmer oligonucleotide of claim 71, wherein the gapmer oligonucleotide comprises sugar modification and internucleoside linkage combination of: eeeee-d10-eeeee and sssssssssssssssssss, wherein e=2′-MOE nucleoside and d=a deoxyribonucleoside, wherein s=a phosphorothioate linkage, wherein at least one “e” or at least one “d” of the gapmer oligonucleotide is replaced with a spacer, and wherein each cytosine of the 2′-MOE nucleosides is a 5-methylcytosine.

105. The gapmer oligonucleotide of any one of the preceding claims, wherein the gapmer oligonucleotide further comprises a conjugate moiety.

106. The gapmer oligonucleotide of claim 105, wherein the conjugate moiety is a cholesterol conjugate located on the 3′ end of the gapmer oligonucleotide.

107. A pharmaceutical composition comprising the gapmer oligonucleotide of any one of claims 1-106, or a pharmaceutically acceptable salt thereof, and a pharmaceutically acceptable excipient.

108. A method of treating a neurological disease in a patient in need thereof, the method comprising administering to the patient a gapmer oligonucleotide of any one of claims 1-106 or a pharmaceutically acceptable salt thereof, or a pharmaceutical composition of claim 107.

109. The method of claim 108, wherein the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

110. The method of claim 108-109, wherein the gapmer oligonucleotide is administered topically, parenterally, intrathecally, orally, pulmonarily, intratracheally, intranasally, transdermally, buccally, intrathalamically, intracerebroventricularly, intraocularly, sublingually, rectally, vaginally, or intraduodenally.

111. The method of claim 110, wherein the gapmer oligonucleotide is administered intrathecally.

112. The method of any one of claims 108-111, wherein a therapeutically effective amount of the gapmer oligonucleotide is administered.

113. The method of any one of claims 108-112, wherein the patient is a human.

114. The pharmaceutical composition of claim 107, wherein the pharmaceutical composition is suitable for topical, parenteral, intrathecal, oral, pulmonary, intratracheal, intranasal, transdermal, buccal, intrathalamical, intracerebroventricular, intraocular, sublingual, rectal, vaginal, or intraduodenal.

115. Use of a gapmer oligonucleotide in the manufacture of a medicament for the treatment of neurological disease.

116. The use of claim 115, wherein the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

117. The use of claim 115 or 116, wherein the gapmer oligonucleotide is the gapmer oligonucleotide of any one of claims 1-106.

118. A method of treating a neurological disease in a patient in need thereof, the method comprising administering to a patient in need thereof a therapeutically effective amount of a pharmaceutical composition comprising a gapmer oligonucleotide, and a pharmaceutically acceptable excipient.

119. The method of claim 118, wherein the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

120. The method of claim 118 or 119, wherein the gapmer oligonucleotide is the gapmer oligonucleotide of any one of claims 1-106, a pharmaceutically acceptable salt thereof, or a pharmaceutical composition of claim 107.

121. The method of any one of claims 118-120, wherein the pharmaceutical composition is administered topically, parenterally, orally, pulmonarily, rectally, buccally, sublingually, vaginally, intratracheally, intranasally, intrathecally, intracisternally, transdermally, or intraduodenally.

122. The method of any one of claims 118-121, wherein the pharmaceutical composition is administered intrathecally.

123. The method of any one of claims 118-122, wherein the patient is human.

124. A gapmer oligonucleotide of any one of claims 1-106, or a pharmaceutically acceptable salt thereof, for use as a medicament.

125. A gapmer oligonucleotide of any one of claims 1-106, or a pharmaceutically acceptable salt thereof, for use in the treatment of a neurological disease.

126. The gapmer oligonucleotide for use of claim 124 or 125, wherein said neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Gaucher's disease, and facial onset sensory and motor neuronopathy, Guam Parkinson-dementia complex, multisystem proteinopathy, Perry disease, and autism.

127. A gapmer oligonucleotide comprising the sequence of any one of SEQ ID NOs: 301692-301742 or the sequences in Tables 4A-4C, or a pharmaceutically acceptable salt thereof;
wherein the gapmer oligonucleotide further comprises:
a gap segment comprising one or more of linked deoxyribonucleosides, 2′-Fluoro Arabino Nucleic Acids (FANA), and Fluoro Cyclohexenyl nucleic acid (F-CeNA);
a 5′ wing region comprising linked nucleosides; and
a 3′ wing region comprising linked nucleosides;
wherein the gapmer oligonucleotide comprises sugar modifications of eeeee-d10-eeeee, wherein e=2′-MOE nucleoside and d=a deoxyribonucleoside, wherein at least one “e” or at least one “d” is replaced with a spacer,
and wherein each cytosine of the 2′-MOE nucleosides is a 5-methylcytosine.

128. The gapmer oligonucleotide of claim 127, wherein at least one internucleoside linkage of the gapmer oligonucleotide is a phosphorothioate linkage.

129. The gapmer oligonucleotide of claim 128, wherein the phosphorothioate internucleoside linkage is in one of a Rp configuration or a Sp configuration.

130. The gapmer oligonucleotide of claim 127 or 128, wherein all internucleoside linkages of the gapmer oligonucleotide are phosphorothioate linkages.

131. The gapmer oligonucleotide of claim 127 or 128, wherein the gapmer oligonucleotide comprises sugar modification and internucleoside linkage combinations, respectively, in any of the following patterns:
a) eeeee-d10-eeeee and sssssooooooooosssss;
b) eeeee-d10-eeeee and ooooosssssssssooooo;
c) eeeee-d10-eeeee and sssssssssssssssssss;
d) eee-d8-eee and sssooooooosss;
e) eee-d8-eee and ooosssssssooo
f) eee-d8-eee and sssssssssssss;
g) eee-d10-eee and sssooooooooosss;
h) eee-d10-eee and ooosssssssssooo;
i) eee-d10-eee and sssssssssssssss;
j) eeee-d10-eeee and ssssooooooooossss;
k) eeee-d10-eeee and oooosssssssssoooo;
l) eeee-d10-eeee and sssssssssssssssss;
m) eeee-d8-eeee and ssssooooooossss,
n) eeee-d8-eeee and oooosssssssoooo,
o) eeee-d8-eeee and sssssssssssssss,
p) eeeeee-d11-eeeeee and ssssssssssssssssssssss;
q) eeeee-d10-eeeee and sososssssssssssosos;
r) eeeee-d10-eeeee and soooossssssssssooos;
s) eeeee-d10-eeeee and soooossssssssssooss;
t) eeeee-d8-eeeee and sossssssssssssoss;
u) eeeee-d10-eeeee and sosssssssssssssosss;
v) eeeeee-d10-eeee and sssssssssssssssssss;
wherein at least one “e” or at least one “d” of the gapmer oligonucleotide is replaced with a spacer.

132. A pharmaceutical composition comprising the antisense gapmer oligonucleotide of any one of claims 126-131, or a pharmaceutically acceptable salt thereof, and a pharmaceutically acceptable excipient.

133. The gapmer oligonucleotide of any one of claims 127-131, wherein the spacer is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

134. The gapmer oligonucleotide of claim 133, wherein the spacer is represented by Formula (X), wherein: 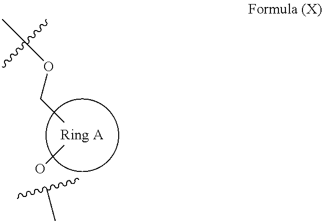
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 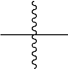
symbol represents the point of connection to an internucleoside linkage.

135. The gapmer oligonucleotide of claim 133, wherein the spacer is represented by Formula (Xa), wherein:

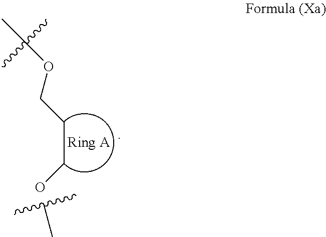

136. The gapmer oligonucleotide of claim 134 or 135, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

137. The method of claim 136, wherein ring A is tetrahydrofuranyl.

138. The method of claim 136, wherein ring A is tetrahydropyranyl.

139. The gapmer oligonucleotide of claim 133, wherein the spacer is represented by Formula (I), wherein: 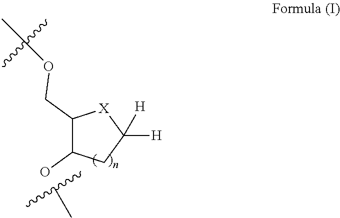
X is selected from —CH₂— and —O—; and
n is 0, 1, 2 or 3.

140. The gapmer oligonucleotide of claim 133, wherein the spacer is represented by Formula (I′), wherein:

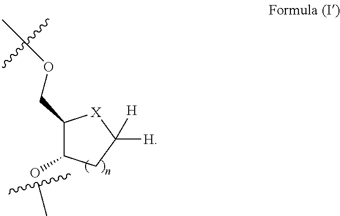

141. The gapmer oligonucleotide of claim 133, wherein the spacer is represented by Formula (Ia), wherein:

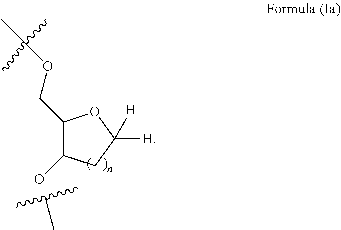

142. The gapmer oligonucleotide of claim 133, wherein the spacer is represented by Formula (Ia′), wherein:

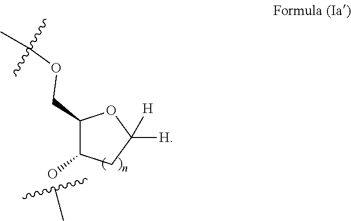

143. The gapmer oligonucleotide of claim 133, wherein the spacer is represented by Formula II, wherein: 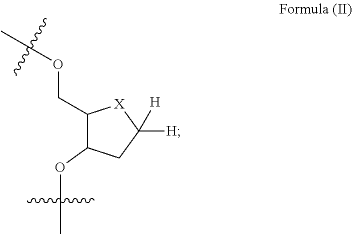
and
X is selected from —CH₂— and —O—.

144. The gapmer oligonucleotide of claim 133, wherein the spacer is represented by Formula II′, wherein: 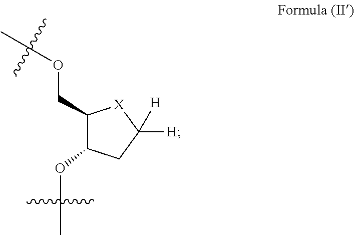
and
X is selected from —CH₂— and —O—.

145. The gapmer oligonucleotide of claim 133, wherein the spacer is represented by Formula (IIa), wherein:

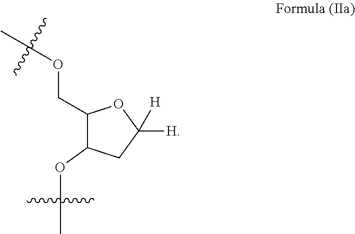

146. The gapmer oligonucleotide of claim 133, wherein the spacer is represented by Formula (Iia′), wherein:

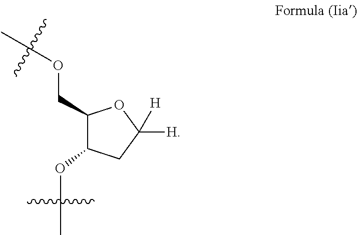

147. The gapmer oligonucleotide of claim 133, wherein the spacer is represented by Formula III, wherein: 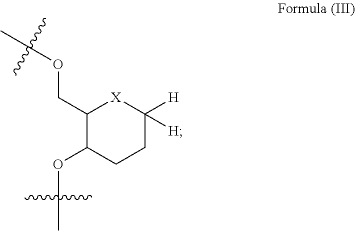
and
X is selected from —CH₂— and —O—.

148. The gapmer oligonucleotide of claim 133, wherein the spacer is represented by Formula III′, wherein: 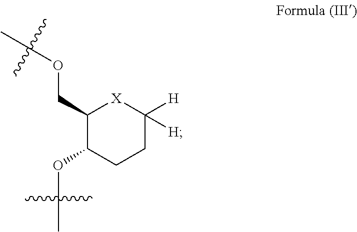
and
X is selected from —CH₂— and —O—.

149. The gapmer oligonucleotide of claim 133, wherein the spacer is represented by Formula (IIIa), wherein:

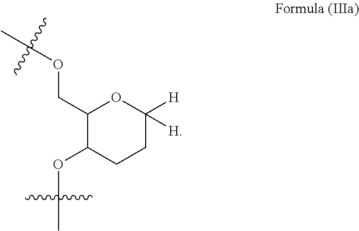

150. The gapmer oligonucleotide of claim 133, wherein the spacer is represented by Formula (IIIa′), wherein:

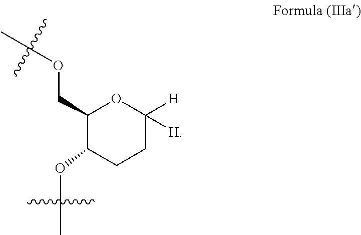

151. A method of treating a neurological disease and/or a neuropathy in a patient in need thereof, the method comprising administering to a patient in need thereof a therapeutically effective amount of a gapmer oligonucleotide of any one of claims 1-106 or claims 127-150 or a pharmaceutically acceptable salt thereof, or a pharmaceutical composition of claim 107 or 132, in combination with a second therapeutic agent selected from a group comprising Riluzole (Rilutek), troriluzole, Edaravone (Radicava), rivastigmine, donepezil, QRL-101, QRL-201, galantamine, selective serotonin reuptake inhibitor, antipsychotic agents, cholinesterase inhibitors, memantine, benzodiazepine antianxiety drugs, AMX0035 (ELYBRIO®), ZILUCOPLAN (RA101495), dual AON intrathecal administration (e.g., BIIB067, BIIB078), BIIB100, levodopa/carbidopa, dopaminergic agents (e.g., ropinirole, pramipexole, rotigotine), medroxyprogesterone, KCNQ2/KCNQ3 openers, Pridopidine, PrimeC (combination of ciprofloxacin and Celebrex), olanzapine (Zyprexa), quetiapine (Seroquel), SSRIs, divalproex sodium (Depakote), carbamazepine (Tegretol), medroxyprogestrone, lithium, anticonvulsants and psychostimulant agents, breathing care, physical therapy, occupational therapy, speech therapy, nutritional support, or any combination thereof.

152. The method of claim 151, wherein the neurological disease is any one of amyotrophic lateral sclerosis (ALS), frontotemporal dementia (FTD), or ALS with FTD.

153. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising Memantine, Rivastigmine, Galantamine, Donepezil, QRL-101, QRL-201, Aricept®, Exelon® (Rivastigmine), Razadyne®, Aducanumab, BAN2401, BIIB091 (gosuranemab), BIIB076, BIIB080 (IONIS-MAPTRx), Elayta (CT1812), MK1942, allogenic hMSC, nilotinib, ABT-957, acitretin, ABT-354, GV1001, Riluzole, CAD106, CNP520, AD-35, Rilapladib, DHP1401, T-817 MA, TC-5619, TPI-287, RVT-101, LY450139, JNJ-54861911, Dapagliflozin, GSK239512, PF-04360365, ASP0777, SB-742457 (a 5-HT6 receptor antagonist), PF-03654746 (an H₃ receptor antagonist), GSK933776 (an Fc-inactivated anti-β amyloid (Aβ) monoclonal antibody (mAb)), Posiphen ((+)-phenserine tartrate), AMX0035 (ELYBRIO®), coenzyme Q10, aducanamab (ADUHLEM), memantine (NAMENDA), Namzeric, Suvorexant (belsomra), lecanemab, or any combination thereof.

154. The method of claim 153, wherein the neurological disease is Alzheimer's Disease.

155. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising Levodopa, Carbidopa-levidopa, pramipexole (MIRAPEX), ropinirole (REQUIP), rotigotine (NEUPRO), apomorphine (APOKYN, KYNMOBI), selegiline (EDLEPRYL, ZELAPAR), rasagiline, entacapone (COMTAN), tolcapone (TASMAR), amantadine (GOCOVRI, SYMMETREL, OSMOLEX), trihexyphenidyl (ARTANE), BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, istradefylline (NOURIANZ), safinamide (XADAGO), benztropine (COGENTIN), opicapone (ongentys), exenatide, lingzhi, Caffeine, sarizotan, embryonic dopamine cell implantation, or any combination thereof.

156. The method of claim 155 wherein the neurological disease is Parkinson's Disease.

157. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising UCB0107, ABBV-8E12, F-18 AV1451, BIIB092, C2N-8E12, tideglusib, deep transcranial magnetic stimulation, lipoic acid, tolfenamica acid, lithium, AZP2006, Glial Clell Line-Derived Neurotrophic Factor, NBMI, suvorxant, zolpidem, TPI 287, davunetide, pimavanserin, Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof.

158. The method of claim 157 wherein the neurological disease is progressive supranuclear palsy (PSP).

159. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising Tetrabenazine, deutetrabenazine, physical therapy, risperidone, haloperidol, chlorpromazine, clonazepam, diazepam, benzodiazepines, selective serotonin reuptake inhibitors. quetiapine, carbatrol, valproate, lamotrigine, pridopidine, delta-9-tetrahydrocannabinol, cannabidiol, stem-cell therapy, ISIS-443139, nilotinib, resveratrol, neflamapimod, fenofibrate, creatine, RO7234292, SAGE-718, WVE-120102, WVE-120101, dimebon, minocycline, deep brain stimulation, ursodiol, coenzyme Q10, OMS643762, VX15/2503, PF-02545920, BN82451B, SEN0014196, olanzapine, tiapridal (tiapride), or any combination thereof.

160. The method of claim 159, wherein the neurological disease is Huntington's Disease.

161. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising anticoagulants, antidepressants, muscle relaxants, stimulants, anticonvulsants, anti-anxiety medication, erythropoietin, hyperbaric treatment, rehabilitation therapies (e.g., physical, occupational, speech, psychological, or vocational counseling), or any combination thereof.

162. The method of claim 161, wherein the neurological disease is brain trauma.

163. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising AXER-204, glyburide, 5-hydroxytryptophan (5-HTP), L-3,4-dihydroxyphenylalanine (L-DOPA), or rehabilitation therapies (e.g., physical therapy, occupational therapy, recreational therapy, use of assistive devices, improved strategies for exercise and healthy diets), or any combination thereof.

164. The method of claim 163, wherein the neurological disease is spinal cord injury.

165. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising TPI-287, lithium, occupational, physical, and speech therapy, or any combination thereof can be selected as an additional therapy.

166. The method of claim 165, wherein the neurological disease is corticobasal degeneration.

167. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising gabapentin, pregabalin, lamotrigine, carbamazepine, duloxetine, gabapentinoids, tricyclic antidepressants, serotonin-norepinephrine reuptake inhibitors, opioids, neurotoxin, dextromethorphan, nicotinamide riboside, auto-antibodies targeting neuronal antigens (TS-HDS and FGFR3), or any combination thereof.

168. The method of claim 167, wherein the neuropathy is a chemotherapy induced neuropathy.

169. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising amantadine, armodafinil, baclofen, buspirone, carbamazepine, citalopram, clonazepam, desvenlafaxine, diazepam, duloxetine, escitalopram, flunarizine, fluoxetine, fluvoxamine, gabapentin, isoniazid, levetiracetam, levodopa, memantine, modafinil, ondansetron, paroxetine, pramipexole, primidone, riluzole, ropinirole, sertraline, tizanidine, topiramate, trihexyphenidyl, valproic acid, venlafaxine, BHV-4157, or a combination thereof.

170. The method of claim 169, wherein the neurological disease is spinocerebellar ataxia.

171. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), zonisamide (zonegran), or any combination thereof.

172. The method of claim 171, wherein the neurological disease is epilepsy.

173. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising nusinersen (SPINRAZA), onasemnogene abeparvovec-xioi (ZOLGENSMA), risdiplam (EVRYSDI), or any combination thereof.

174. The method of claim 173, wherein the neurological disease is spinal muscular atrophy.

175. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising anti-seizure medications, speech therapy, physical therapy, occupational therapy, Adrabetadex, Arimoclomol, N-Acetyl-L-Leucine, or any combination thereof.

176. The method of claim 175, wherein the neurological disease is Niemann-Pick disease type C.

177. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising physical and occupational therapies, orthopedic surgery, orthopedic devices, PXT3003, or any combination thereof.

178. The method of claim 177, wherein the neurological disease is Charcot-Marie-Tooth Disease (CMT).

179. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising enzyme replacement therapy: idursulfase (Elaprase), surgical intervention (tonsillectomy and/or adenoidectomy), RGX-121 gene therapy, adalimumab, MT2013-31, or any combination thereof.

180. The method of claim 179, wherein the neurological disease is Mucopolysaccharidosis type II (MPSIIA).

181. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising physical, occupational, and speech therapies, contact lenses and artificial tears, genetic counseling, or any combination thereof.

182. The method of claim 181, wherein the neurological disease is Mucolipidosis IV.

183. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising anticonvulsants, physical and occupational therapies, galactosidase, gene delivery of galactosidase, LYS-GM101 gene therapy, or any combination thereof.

184. The method of claim 183, wherein the neurological disease is GM1 gangliosidosis.

185. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising physical and occupational therapies, use of devices such as braces, walkers, wheelchairs, immunosuppressants, BYM338, or any combination thereof.

186. The method of claim 185, wherein the neurological disease is Sporadic inclusion body myositis (sIBM).

187. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising corticosteroids, colchicine, dapsone, azathioprine, or any combination thereof.

188. The method of claim 187, wherein the neurological disease is Henoch-Schonlein purpura (HSP).

189. A method of treating a neurological disease and/or a neuropathy in a patient in need thereof, the method comprising administering to a patient in need thereof a therapeutically effective amount of a gapmer oligonucleotide of any one of claims 1-106 or claims 127-130 or a pharmaceutically acceptable salt thereof, or a pharmaceutical composition of claim 107 or 132, in combination with a second therapeutic agent selected from a group comprising enzyme replacement therapy, substrate reduction therapy, N-acetylcysteine, GZ/SAR402671, cerezyme, or any combination thereof.

190. The method of claim 189, wherein the neurological disease is Gaucher's disease.
